(19) United States
(12) Patent Application Publication
(10) Pub. No.: US 2026/0209369 A1
(43) Pub. Date: Jul. 23, 2026

(54) ANTI-TL1A x ANTI-IL23 ANTIBODIES

(71) Applicant: Xencor, Inc., Pasadena, CA (US)

(72) Inventors: James Ernst, Pasadena, CA (US); Matthew S. Faber, Glendora, CA (US); Gregory Moore, Azusa, CA (US); Kendra N. Avery, Hawthorne, CA (US); Juan Diaz, Anaheim Hills, CA (US); Peter Donghyun Nam, Monterey Park, CA (US); Jitendra Kanodia, Foster City, CA (US); Viralkumar Rameshkumar Davra, Chino, CA (US); James Scott Wieler, Pasadena, CA (US); Jing Qi, Arcadia, CA (US); Chi Nguyen, Garden Grove, CA (US); Thuy B. Truong, Baldwin Park, CA (US); Su-Shin Hao, Pasadena, CA (US)

(21) Appl. No.: 19/323,363

(22) Filed: Sep. 9, 2025

Related U.S. Application Data
(60) Provisional application No. 63/692,669, filed on Sep. 9, 2024.
(60) Provisional application No. 63/761,754, filed on Feb. 21, 2025.
(60) Provisional application No. 63/796,313, filed on Apr. 28, 2025.

(51) Int. Cl. C07K 16/28; A61K 39/00; A61K 39/395; C07K 16/24

(52) U.S. Cl. CPC C07K 16/2875; A61K 39/3955; C07K 16/244; A61K 2039/505; C07K 2317/24; C07K 2317/31; C07K 2317/35; C07K 2317/52; C07K 2317/55; C07K 2317/565; C07K 2317/622; C07K 2317/76; C07K 2317/92; C07K 2317/94

(57) ABSTRACT

Provided herein are novel anti-TL1A antibodies, anti-IL23 antibodies, and anti-TL1A×anti-IL23 antibodies and methods of using such antibodies for the treatment of TL1A- and IL23-associated diseases (e.g., inflammatory diseases).

### CROSS-REFERENCE TO RELATED APPLICATIONS

This application claims the benefit of U.S. Application No. 63/692,669, filed Sep. 9, 2024, U.S. Application No. 63/761,754, filed Feb. 21, 2025, and U.S. Application No. 63/796,313, filed Apr. 28, 2025, each of which is herein incorporated by reference in its entirety for all purposes.

### REFERENCE TO A SEQUENCE LISTING SUBMITTED AS AN XML FILE

The Sequence Listing written in file 636745SEQLIST.xml is 2,599,626 bytes, was created on Sep. 9, 2025, and is hereby incorporated by reference in its entirety.

### BACKGROUND

Antibody-based therapeutics have been used successfully to treat a variety of diseases. There remains a need for novel therapeutics for the treatment of diseases, particularly those that target TNF-like ligand 1A (TL1A) and/or IL23.

### SUMMARY

Provided herein are novel anti-TL1A×anti-IL23 antibodies and methods of using such antibodies for the treatment of TL1A- and/or IL23-associated diseases. Also provided are novel anti-TL1A antibodies and methods of using such antibodies for the treatment of TL1A-associated diseases, novel anti-TL23 antibodies and methods of using such antibodies for the treatment of IL23-associated diseases, and combinations of anti-TL1A antibodies and anti-IL23 antibodies and methods of using such antibodies for the treatment of TL1A- and/or IL23-associated diseases.

In one aspect provided herein is a bispecific anti-TNF-like ligand 1A (TL1A)×anti-interleukin-23 (IL23) bispecific antibody comprising: a) a means for binding TL1A; and b) a means for binding IL23.

In another aspect provided herein is a bispecific anti-TL1A×anti-IL23 antibody comprising: a) a TL1A binding domain comprising i) a first variable heavy domain (VH1), and ii) a first variable light domain (VL1); and b) a IL23 binding domain comprising i) a second variable heavy domain (VH2), and ii) a second variable light domain (VL2). In some embodiments the TL1A binding domain comprises the VH and VL of any of the TL1A binding domains in Tables 7-10. In some embodiments, the IL23 binding domain comprises the VH and VL of IL23-A[IL23]_H1L1 (Table 11).

In another aspect, provided herein is a heterodimeric antibody that comprises: a) a first monomer; b) a second monomer; and c) a light chain. The first monomer comprises: i) a single chain variable fragment (scFv); and ii) a first Fc domain, wherein the scFv is covalently attached to the N-terminus of the first Fc domain using a domain linker. The second monomer comprises, from N-terminal to C-terminal, a VH1-CH1-hinge-CH2-CH3, wherein VH1 is a first variable heavy domain and CH2-CH3 is a second Fc domain. The light chain comprises, from N-terminal to C-terminal, VL1-CL, wherein VL1 is a first variable light domain and CL is a constant light domain. The scFv comprises a second VH domain (VH2), a scFv linker, and a second variable light domain (VL2). Further, the VH1 and the VL1 together form a first antigen-binding domain (ABD) and the VH2 and the VL2 together form a second ABD, and one of the first ABD and second ABD is a TL1A binding domain and the other of the first ABD and second ABD is a IL23 binding domain.

In some embodiments, the scFv comprises, from N-terminal to C-terminal, VH2-scFv linker-VL2. In other embodiments, the scFv comprises, from N-terminal to C-terminal, VL2-scFv linker-VH2.

In some embodiments, the first ABD is the TL1A binding domain and the second ABD is the IL23 binding domain. In some embodiments, the first ABD is the IL23 binding domain and the second ABD is the TL1A binding domain. In some embodiments, the TL1A binding domain comprises the VH and VL of any of the TL1A binding domains in Tables 7-10. In certain embodiments, the IL23 binding domain comprises the VH and VL of IL23-A[IL23]_H1L1 (Table 11).

In another aspect, provided herein is a heterodimeric antibody that comprises: a) a first monomer; b) a second monomer; c) a first light chain; and d) a second light chain. The first monomer comprises a VH1-CH1-hinge-CH2-CH3, wherein VH1 is a first variable heavy domain and CH2-CH3 is a first Fc domain. The second monomer comprises a VH2-CH1-hinge-CH2-CH3, wherein VH2 is a second variable heavy domain and CH2-CH3 is a second Fc domain. The first light chain comprises a VL1-CL, wherein VL1 is a first variable light domain. The second light chain comprises a VL2-CL, wherein VL2 is a second variable light domain. The VH1 and the VL1 together form a first antigen-binding domain (ABD) and the VH2 and the VL2 together form a second ABD. One of the first ABD and second ABD is a TL1A binding domain and the other of the first ABD and second ABD is a IL23 binding domain. Further, i) CH1 of the first monomer and CL1 of the first light chain comprise a set of electrostatic steering variants as depicted in FIG. 4 or Table 27 or a set of steric skew variants as depicted in FIG. 5 or Table 28; ii) VH1 of the first monomer and VL1 of the first light chain comprise a set of electrostatic steering variants as depicted in FIG. 6 or Tables 18 and 19; iii) CH1 of the second monomer and CL1 of the second light chain comprise a set of electrostatic steering variants as depicted in FIG. 4 or Table 27 or a set of steric skew variants as depicted in FIG. 5 or Table 28; and/or iv) VH2 of the second monomer and VL2 of the second light chain comprise a set of electrostatic steering variants as depicted in FIG. 6 or Tables 18 and 19.

In some embodiments, the CH1 of the first monomer and CL1 of the first light chain comprise a set of electrostatic steering variants as depicted in FIG. 4 or Table 27. In some embodiments, the CH1 of the first monomer and CL1 of the first light chain comprise a set of steric skew variants as depicted in FIG. 5 or Table 28. In exemplary embodiments, the VH1 of the first monomer and VL1 of the first light chain comprise a set of electrostatic steering variants as depicted in FIG. 6 or Tables 18 and 19. In some embodiments, the CH1 of the second monomer and CL1 of the second light chain comprise a set of electrostatic steering variants as depicted in FIG. 4 or Table 27. In some embodiments, the CH1 of the second monomer and CL1 of the second light chain comprise a set of steric skew variants as depicted in FIG. 5 or Table 28. In exemplary embodiments, the VH2 of the second monomer and VL2 of the second light chain comprise a set of electrostatic steering variants as depicted in FIG. 6 or Tables 18 and 19.

In some embodiments, VH1 comprises amino acid substitution Q39E and VL1 comprises amino acid substitution Q38K (Kabat numbering). In some embodiments, VH1 comprises amino acid substitution Q39K and VL1 comprises amino acid substitution Q38E (Kabat numbering). In exemplary embodiments, VH1 comprises Q39E, VL1 comprises Q38K, VH2 comprises Q39K and VL2 comprises Q38E (Kabat numbering).

In some embodiments, CH1 of the first monomer comprises amino acid substitutions K213E/K218D and CL comprises amino acid substitutions D122K/E123K (EU numbering). In some embodiments, CH1 of the first monomer comprises amino acid substitutions A141F and CL comprises amino acid substitution F118A (EU numbering). In exemplary embodiments, CH1 of the first monomer comprises amino acid substitutions K213E/K218D, CL of the first light chain comprises amino acid substitutions D122K/E123K, CH1 of the second monomer comprises amino acid substitutions A141F and CL of the second light chain comprises amino acid substitution F118A (EU numbering).

In some embodiments, VH1 comprises amino acid substitution Q39E (Kabat numbering), VL1 comprises amino acid substitution Q38K (Kabat numbering), CH1 of the first monomer comprises amino acid substitutions K213E/K218D (EU numbering), CL of the first light chain comprises amino acid substitutions D122K/E123K (EU numbering), VH2 comprises amino acid substitution Q39K (Kabat numbering), VL2 comprises amino acid substitution Q38E (Kabat numbering), CH1 of the second monomer comprises amino acid substitutions A141F (EU numbering) and CL of the second light chain comprises amino acid substitution F118A (EU numbering).

In exemplary embodiments, the first ABD is the TL1A binding domain and the second ABD is the IL23 binding domain. In some embodiments, the first ABD is the IL23 binding domain and the second ABD is the TL1A binding domain. In certain embodiments, the TL1A binding domain comprises the VH and VL of any of the TL1A binding domains in Tables 7-10. In some embodiments, the IL23 binding domain comprises the VH and VL of IL23-A[IL23]_H1L1 (Table 11).

In another aspect provided herein is a heterodimeric antibody comprising: a) a first monomer; b) a second monomer; c) a first common light chain; and d) a second common light chain. The first monomer comprises a VH1-CH1-hinge-CH2-CH3, wherein VH1 is a first variable heavy domain and CH2-CH3 is a first Fc domain. The second monomer comprises a VH2-CH1-hinge-CH2-CH3, wherein VH2 is a second variable heavy domain and CH2-CH3 is a second Fc domain. Each of the first and second common light chains comprise a VL-CL. The VH1 and the VL of the first common light chain together form a first antigen-binding domain (ABD) and the VH2 and the VL of the second common light chain together form a second ABD.

In some embodiments, the first ABD is the TL1A binding domain and the second ABD is the IL23 binding domain. In other embodiments, the first ABD is the IL23 binding domain and the second ABD is the TL1A binding domain.

In some embodiments, the TL1A binding domain comprises the VH and VL of any of the TL1A binding domains in Tables 7-10. In some embodiments, the IL23 binding domain comprises the VH and VL of IL23-A[IL23]_H1L1 (Table 11).

In another aspect, provided herein is a heterodimeric antibody that comprises: a) a first monomer; b) a second monomer; c) a first light chain; and d) a second light chain. The first monomer comprises: i) a VH1-CH1-hinge-CH2-CH3, and ii) a first scFv comprising a VH2-scFv linker-VL2, wherein the scFv is covalently attached to the CH3 of the first monomer by a linker. The second monomer comprises: i) a VH1-CH1-hinge-CH2-CH3, and ii) a second scFv comprising a VH2-scFv linker-VL2, wherein the scFv is covalently attached to the CH3 of the second monomer by a linker. Each of the first and second light chains comprise a VL1-CL. Each of the VH1s of the first monomer and second monomer are a first variable heavy domain, each of the VL1s of the first monomer and second monomer are a first light domain, each of the VH2s are a second variable heavy domain, and each of the VL2s are a second variable light domain. Further, the VH1 of the first monomer and VL1 of the first light chain, and the VH1 of the second monomer and VL1 of the second light chain each form a first antigen-binding domain, and the VH2 and VL2 of the first and second scFvs each for a second antigen-binding domain, wherein the first ABDs or the second ABDs are TL1A binding domains and the other of the first ABDs and second ABDs are IL23 binding domains.

In some embodiments, the first and second scFvs each comprise, from N-terminal to C-terminal, VH2-scFv linker-VL2. In other embodiments, the first and second scFvs each comprise, from N-terminal to C-terminal, VL2-scFv linker-VH2.

In some embodiments, the first ABDs are the TL1A binding domains and the second ABDs are the IL23 binding domain. In other embodiments, the first ABD is the IL23 binding domain and the second ABD is the TL1A binding domain. In some embodiments, the TL1A binding domain comprises the VH and VL of any of the TL1A binding domains in Tables 7-10. In some embodiments, the IL23 binding domain comprises the VH and VL of IL23-A[IL23]_H1L1 (Table 11).

In some embodiments of the heterodimeric antibodies provided herein, the first Fc domain and second Fc domain are each variant Fc domains. In some embodiments, the first and second Fc domains comprise a set of heterodimerization skew variants selected from the following heterodimerization variants: S364K/E357Q:L368D/K370S; S364K:L368D/K370S; S364K:L368E/K370S; D401K:T411E/K360E/Q362E; and T366W:T366S/L368A/Y407V, wherein numbering is according to EU numbering. In exemplary embodiments, the first and second Fc domains comprise heterodimerization skew variants S364K/E357Q:L368D/K370S, wherein numbering is according to EU numbering.

In some embodiments, the first and second Fc domains each comprise one or more ablation variants. In some embodiments, the one or more ablation variants comprise E233P/L234V/L235A/G236del/S267K, wherein numbering is according to EU numbering.

In some embodiments, one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421 D, the first Fc domain comprises amino acid variants E233P/L234V/L235A/G236del/S267K/S364K/E357Q, and wherein numbering is according to EU numbering.

In some embodiments, the first and second variant Fc domains each comprise amino acid variants 428L/434S.

In some embodiments wherein the heterodimeric antibody includes an scFv with an scFv linker, the scFv linker has the amino acid sequence GKPGSGKPGSGKPGSGKPGS (SEQ ID NO: 16).

Also provided herein are nucleic acid and expression vector compositions encoding the subject antibodies, host cells comprising the expression vector compositions, and methods of making the subject antibodies.

In another aspect, provided herein is a method of treating a TL1A-associated and/or IL23 associated disease in a patient in need thereof, comprising administering to the patient a subject anti-TL1A×anti-IL23 antibody described herein.

In one aspect, provided are bispecific or heterodimeric antibodies comprising: (a) a TL1A-binding domain comprising a first variable heavy domain (VH1) and a first variable light domain (VL1); and (b) an IL23-binding domain comprising a second variable heavy domain (VH2) and a second variable light domain (VL2).

In some such antibodies, the VH1 comprises a vhCDR1, a vhCDR2, and a vhCDR3 and the VL1 comprises a vlCDR1, a vlCDR2, and a vlCDR3 contained within any pair of anti-TL1A variable heavy domain and variable light domain in Table 44, optionally wherein the CDRs are as defined by the Kabat, Chothia, Kabat+Chothia, AbM, contact, or IMGT definition. In some such antibodies, the VH1 comprises a vhCDR1, a vhCDR2, and a vhCDR3 and the VL1 comprises a vlCDR1, a vlCDR2, and a vlCDR3 contained within any pair of anti-TL1A variable heavy domain and variable light domain in Tables 7-10, 22, 23, 26, 29-34, and 48, optionally wherein the CDRs are as defined by the Kabat, Chothia, Kabat+Chothia, AbM, contact, or IMGT definition. In some such antibodies, the VH1 comprises a vhCDR1, a vhCDR2, and a vhCDR3 contained within a variable heavy domain comprising the amino acid sequence of any one of SEQ ID NOS: 1427-1823 and 2316-2330, and the VL1 comprises a vlCDR1, a vlCDR2, and a vlCDR3 contained within a variable light domain comprising the amino acid sequence of any one of 1824-1923, optionally wherein the CDRs are as defined by the Kabat, Chothia, Kabat+Chothia, AbM, contact, or IMGT definition. In some such antibodies, the VH1 and VL1 comprise any pair of anti-TL1A variable heavy domain and variable light domain in Table 44, or variants thereof in which an N-terminal glutamine or glutamate is replaced with pyroglutamate. In some such antibodies, the VH1 and VL1 comprise any pair of anti-TL1A variable heavy domain and variable light domain in Tables 7-10, 22, 23, 26, 29-34, and 48, or variants thereof in which an N-terminal glutamine or glutamate is replaced with pyroglutamate. In some such antibodies, the VH1 and VL1 comprise a variable heavy domain comprising the amino acid sequence of any one of SEQ ID NOS: 1427-1823 and 2316-2330, and a variable light domain comprising the amino acid sequence of any one of 1824-1923, or variants thereof in which an N-terminal glutamine or glutamate is replaced with pyroglutamate.

In some such antibodies, the VH2 comprises a vhCDR1, a vhCDR2, and a vhCDR3 and the VL2 comprises a vlCDR1, a vlCDR2, and a vlCDR3 contained within any pair of anti-TL1A variable heavy domain and variable light domain in Table 44, optionally wherein the CDRs are as defined by the Kabat, Chothia, Kabat+Chothia, AbM, contact, or IMGT definition. In some such antibodies, the VH2 comprises a vhCDR1, a vhCDR2, and a vhCDR3 and the VL2 comprises a vlCDR1, a vlCDR2, and a vlCDR3 contained within any pair of anti-TL1A variable heavy domain and variable light domain in Tables 11, 24-26, 29-34, and 49, optionally wherein the CDRs are as defined by the Kabat, Chothia, Kabat+Chothia, AbM, contact, or IMGT definition. In some such antibodies, the VH2 comprises a vhCDR1, a vhCDR2, and a vhCDR3 contained within a variable heavy domain comprising the amino acid sequence of any one of 1927-2132, and the VL2 comprises a vlCDR1, a vlCDR2, and a vlCDR3 contained within a variable light domain comprising the amino acid sequence of any one of 2133-2232, optionally wherein the CDRs are as defined by the Kabat, Chothia, Kabat+Chothia, AbM, contact, or IMGT definition. In some such antibodies, the VH2 and VL2 comprise any pair of anti-IL23 variable heavy domain and variable light domain in Table 44 or variants thereof in which an N-terminal glutamine or glutamate is replaced with pyroglutamate. In some such antibodies, the VH2 and VL2 comprise any pair of anti-IL23 variable heavy domain and variable light domain in Tables 11, 24-26, 29-34, and 49 or variants thereof in which an N-terminal glutamine or glutamate is replaced with pyroglutamate. In some such antibodies, the VH2 and VL2 comprise a variable heavy domain comprising the amino acid sequence of any one of 1927-2132, and a variable light domain comprising the amino acid sequence of any one of 2133-2232 or variants thereof in which an N-terminal glutamine or glutamate is replaced with pyroglutamate.

In some such antibodies, the VH1 and the VL1 comprise a variable heavy domain comprising a vhCDR1, a vhCDR2, and a vhCDR3 contained within a variable heavy domain comprising the amino acid sequence of any one of SEQ ID NOS: 2241, 2253, 2259, 1674, and 1200, and a variable light domain comprising a vlCDR1, a vlCDR2, and a vlCDR3 contained within a variable light domain comprising the amino acid sequence of any one of SEQ ID NOS: 2243 and 1204. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 2241, and a variable light domain comprising a vlCDR1, a vlCDR2, and a vlCDR3 contained within a variable light domain comprising the amino acid sequence of SEQ ID NO: 2243. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 2253, and a variable light domain comprising a vlCDR1, a vlCDR2, and a vlCDR3 contained within a variable light domain comprising the amino acid sequence of SEQ ID NO: 2243. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 2259, and a variable light domain comprising a vlCDR1, a vlCDR2, and a vlCDR3 contained within a variable light domain comprising the amino acid sequence of SEQ ID NO: 2243. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 1674, and a variable light domain comprising a vlCDR1, a vlCDR2, and a vlCDR3 contained within a variable light domain comprising the amino acid sequence of SEQ ID NO: 1204. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 1200, and a variable light domain comprising a vlCDR1, a vlCDR2, and a vlCDR3 contained within a variable light domain comprising the amino acid sequence of SEQ ID NO: 1204. In some such antibodies, the VH1 and the VL1 comprise a variable heavy domain comprising a vhCDR1, a vhCDR2, and a vhCDR3 contained within a variable heavy domain comprising the amino acid sequence of any one of SEQ ID NOS: 1427-1823 and 2316-2330, and a variable light domain comprising a vlCDR1, a vlCDR2, and a vlCDR3 contained within a variable light domain comprising the amino acid sequence of any one of SEQ ID NOS: 1824-1923. In some such antibodies, the VH1 and the VL1 comprise a variable heavy domain comprising a vhCDR1, a vhCDR2, and a vhCDR3 contained within a variable heavy domain comprising the amino acid sequence of any one of SEQ ID NOS: 1674, 1200, 1661, and 2323, and a variable light domain comprising a vlCDR1, a vlCDR2, and a vlCDR3 contained within a variable light domain comprising the amino acid sequence of SEQ ID NO: 1204. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 1661, and a variable light domain comprising a vlCDR1, a vlCDR2, and a vlCDR3 contained within a variable light domain comprising the amino acid sequence of SEQ ID NO: 1204. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 2323, and a variable light domain comprising a vlCDR1, a vlCDR2, and a vlCDR3 contained within a variable light domain comprising the amino acid sequence of SEQ ID NO: 1204. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 101, and a variable light domain comprising a vlCDR1, a vlCDR2, and a vlCDR3 contained within a variable light domain comprising the amino acid sequence of SEQ ID NO: 105. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 109, and a variable light domain comprising a vlCDR1, a vlCDR2, and a vlCDR3 contained within a variable light domain comprising the amino acid sequence of SEQ ID NO: 113. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 117, and a variable light domain comprising a vlCDR1, a vlCDR2, and a vlCDR3 contained within a variable light domain comprising the amino acid sequence of SEQ ID NO: 121. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 125, and a variable light domain comprising a vlCDR1, a vlCDR2, and a vlCDR3 contained within a variable light domain comprising the amino acid sequence of SEQ ID NO: 129. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 133, and a variable light domain comprising a vlCDR1, a vlCDR2, and a vlCDR3 contained within a variable light domain comprising the amino acid sequence of SEQ ID NO: 137. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 141, and a variable light domain comprising a vlCDR1, a vlCDR2, and a vlCDR3 contained within a variable light domain comprising the amino acid sequence of SEQ ID NO: 145. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 149, and a variable light domain comprising a vlCDR1, a vlCDR2, and a vlCDR3 contained within a variable light domain comprising the amino acid sequence of SEQ ID NO: 153. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 157, and a variable light domain comprising a vlCDR1, a vlCDR2, and a vlCDR3 contained within a variable light domain comprising the amino acid sequence of SEQ ID NO: 159. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 161, and a variable light domain comprising a vlCDR1, a vlCDR2, and a vlCDR3 contained within a variable light domain comprising the amino acid sequence of SEQ ID NO: 165. In some such antibodies, the VH1 and the VL1 comprise a variable heavy domain comprising a vhCDR1, a vhCDR2, and a vhCDR3 contained within a variable heavy domain comprising the amino acid sequence of SEQ ID NO: 169, and a variable light domain comprising a vlCDR1, a vlCDR2, and a vlCDR3 contained within a variable light domain comprising the amino acid sequence of SEQ ID NO: 173. In some such antibodies, the VH1 and the VL1 comprise a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH1 and the VL1 comprise a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in Tables 29 and 31-34. In any of the above, the CDRs are optionally as defined by the Kabat, Chothia, Kabat+Chothia, AbM, contact, or IMGT definition.

In some such antibodies, the VH1 and the VL1 comprise a variable heavy domain comprising a vhCDR1 comprising the amino acid sequence of SEQ ID NO: 102 or 1201, a vhCDR2 comprising the amino acid sequence of SEQ ID NO: 1924 or 1202, and a vhCDR3 comprising the amino acid sequence of SEQ ID NO: 1203 or 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and the VL1 comprise a variable heavy domain comprising a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and the VL1 comprise a variable heavy domain comprising a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and the VL1 comprise a variable heavy domain comprising a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and the VL1 comprise a variable heavy domain comprising a vhCDR1 comprising the amino acid sequence of SEQ ID NO: 102, a vhCDR2 comprising the amino acid sequence of SEQ ID NO: 1926, and a vhCDR3 comprising the amino acid sequence of SEQ ID NO: 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and the VL1 comprise a variable heavy domain comprising a vhCDR1 comprising the amino acid sequence of SEQ ID NO: 102, a vhCDR2 comprising the amino acid sequence of SEQ ID NO: 103, and a vhCDR3 comprising the amino acid sequence of SEQ ID NO: 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and the VL1 comprise a variable heavy domain comprising a vhCDR1 comprising the amino acid sequence of SEQ ID NO: 110, a vhCDR2 comprising the amino acid sequence of SEQ ID NO: 111, and a vhCDR3 comprising the amino acid sequence of SEQ ID NO: 112, and a variable light domain comprising a vlCDR1 comprising the amino acid sequence of SEQ ID NO: 114, a vlCDR2 comprising the amino acid sequence of SEQ ID NO: 115, and a vlCDR3 comprising the amino acid sequence of SEQ ID NO: 116. In some such antibodies, the VH1 and the VL1 comprise a variable heavy domain comprising a vhCDR1 comprising the amino acid sequence of SEQ ID NO: 118, a vhCDR2 comprising the amino acid sequence of SEQ ID NO: 119, and a vhCDR3 comprising the amino acid sequence of SEQ ID NO: 120, and a variable light domain comprising a vlCDR1 comprising the amino acid sequence of SEQ ID NO: 122, a vlCDR2 comprising the amino acid sequence of SEQ ID NO: 123, and a vlCDR3 comprising the amino acid sequence of SEQ ID NO: 124. In some such antibodies, the VH1 and the VL1 comprise a variable heavy domain comprising a vhCDR1 comprising the amino acid sequence of SEQ ID NO: 126, a vhCDR2 comprising the amino acid sequence of SEQ ID NO: 127, and a vhCDR3 comprising the amino acid sequence of SEQ ID NO: 128, and a variable light domain comprising a vlCDR1 comprising the amino acid sequence of SEQ ID NO: 130, a vlCDR2 comprising the amino acid sequence of SEQ ID NO: 131, and a vlCDR3 comprising the amino acid sequence of the amino acid sequence of SEQ ID NO: 132. In some such antibodies, the VH1 and the VL1 comprise a variable heavy domain comprising a vhCDR1 comprising the amino acid sequence of SEQ ID NO: 134, a vhCDR2 comprising the amino acid sequence of SEQ ID NO: 135, and a vhCDR3 comprising the amino acid sequence of SEQ ID NO: 136, and a variable light domain comprising a vlCDR1 comprising the amino acid sequence of SEQ ID NO: 138, a vlCDR2 comprising the amino acid sequence of SEQ ID NO: 139, and a vlCDR3 comprising the amino acid sequence of the amino acid sequence of SEQ ID NO: 140. In some such antibodies, the VH1 and the VL1 comprise a variable heavy domain comprising a vhCDR1 comprising the amino acid sequence of SEQ ID NO: 142, a vhCDR2 comprising the amino acid sequence of SEQ ID NO: 143, and a vhCDR3 comprising the amino acid sequence of SEQ ID NO: 144, and a variable light domain comprising a vlCDR1 comprising the amino acid sequence of SEQ ID NO: 146, a vlCDR2 comprising the amino acid sequence of SEQ ID NO: 147, and a vlCDR3 comprising the amino acid sequence of SEQ ID NO: 148. In some such antibodies, the VH1 and the VL1 comprise a variable heavy domain comprising a vhCDR1 comprising the amino acid sequence of SEQ ID NO: 150, a vhCDR2 comprising the amino acid sequence of SEQ ID NO: 151, and a vhCDR3 comprising the amino acid sequence of SEQ ID NO: 152, and a variable light domain comprising a vlCDR1 comprising the amino acid sequence of SEQ ID NO: 154, a vlCDR2 comprising the amino acid sequence of SEQ ID NO: 155, and a vlCDR3 comprising the amino acid sequence of SEQ ID NO: 156. In some such antibodies, the VH1 and the VL1 comprise a variable heavy domain comprising a vhCDR1 comprising the amino acid sequence of SEQ ID NO: 118, a vhCDR2 comprising the amino acid sequence of SEQ ID NO: 158, and a vhCDR3 comprising the amino acid sequence of SEQ ID NO: 120, and a variable light domain comprising a vlCDR1 comprising the amino acid sequence of SEQ ID NO: 122, a vlCDR2 comprising the amino acid sequence of SEQ ID NO: 123, and a vlCDR3 comprising the amino acid sequence of SEQ ID NO: 160. In some such antibodies, the VH1 and the VL1 comprise a variable heavy domain comprising a vhCDR1 comprising the amino acid sequence of SEQ ID NO: 162, a vhCDR2 comprising the amino acid sequence of SEQ ID NO: 163, and a vhCDR3 comprising the amino acid sequence of SEQ ID NO: 164, and a variable light domain comprising a vlCDR1 comprising the amino acid sequence of SEQ ID NO: 166, a vlCDR2 comprising the amino acid sequence of SEQ ID NO: 167, and a vlCDR3 comprising the amino acid sequence of SEQ ID NO: 168. In some such antibodies, the VH1 and the VL1 comprise a variable heavy domain comprising a vhCDR1 comprising the amino acid sequence of SEQ ID NO: 170, a vhCDR2 comprising the amino acid sequence of SEQ ID NO: 171, and a vhCDR3 comprising the amino acid sequence of SEQ ID NO: 172, and a variable light domain comprising a vlCDR1 comprising the amino acid sequence of SEQ ID NO: 174, a vlCDR2 comprising the amino acid sequence of SEQ ID NO: 175, and a vlCDR3 comprising the amino acid sequence of SEQ ID NO: 176.

In some such antibodies, the VH1 is at least 90%, at least 95%, at least 96%, at least 97%, at least 98%, or at least 99% identical to the amino acid sequence of any one of SEQ ID NOS: 2241, 2253, 2259, 1674, and 1200, and the VL1 is at least 90%, at least 95%, at least 96%, at least 97%, at least 98%, or at least 99% identical to the amino acid sequence of any one of SEQ ID NOS: 2243 and 1204. In some such antibodies, the VH1 is at least 90%, at least 95%, at least 96%, at least 97%, at least 98%, or at least 99% identical to the amino acid sequence of SEQ ID NO: 2241, and the VL1 is at least 90%, at least 95%, at least 96%, at least 97%, at least 98%, or at least 99% identical to the amino acid sequence of SEQ ID NO: 2243. In some such antibodies, the VH1 is at least 90%, at least 95%, at least 96%, at least 97%, at least 98%, or at least 99% identical to the amino acid sequence of SEQ ID NO: 2253, and the VL1 is at least 90%, at least 95%, at least 96%, at least 97%, at least 98%, or at least 99% identical to the amino acid sequence of SEQ ID NO: 2243. In some such antibodies, the VH1 is at least 90%, at least 95%, at least 96%, at least 97%, at least 98%, or at least 99% identical to the amino acid sequence of SEQ ID NO: 2259, and the VL1 is at least 90%, at least 95%, at least 96%, at least 97%, at least 98%, or at least 99% identical to the amino acid sequence of SEQ ID NO: 2243. In some such antibodies, the VH1 is at least 90%, at least 95%, at least 96%, at least 97%, at least 98%, or at least 99% identical to the amino acid sequence of SEQ ID NO: 1674, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of SEQ ID NO: 1200, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of any one of SEQ ID NOS: 1427-1823 and 2316-2330, and the VL1 is at least 90%, at least 95%, at least 96%, at least 97%, at least 98%, or at least 99% identical to the amino acid sequence of any one of SEQ ID NOS: 1824-1923. In some such antibodies, the VH1 is at least 90%, at least 95%, at least 96%, at least 97%, at least 98%, or at least 99% identical to the amino acid sequence of any one of SEQ ID NOS: 1674, 1200, 1661, and 2323, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of SEQ ID NO: 1661, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of SEQ ID NO: 2323, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of SEQ ID NO: 101, and the VL1 is at least 90%, at least 95%, at least 96%, at least 97%, at least 98%, or at least 99% identical to the amino acid sequence of SEQ ID NO: 105. In some such antibodies, the VH1 is at least 90%, at least 95%, at least 96%, at least 97%, at least 98%, or at least 99% identical to the amino acid sequence of SEQ ID NO: 109, and the VL1 is at least 90%, at least 95%, at least 96%, at least 97%, at least 98%, or at least 99% identical to the amino acid sequence of SEQ ID NO: 113. In some such antibodies, the VH1 is at least 90%, at least 95%, at least 96%, at least 97%, at least 98%, or at least 99% identical to the amino acid sequence of SEQ ID NO: 117, and the VL1 is at least 90%, at least 95%, at least 96%, at least 97%, at least 98%, or at least 99% identical to the amino acid sequence of SEQ ID NO: 121. In some such antibodies, the VH1 is at least 90%, at least 95%, at least 96%, at least 97%, at least 98%, or at least 99% identical to the amino acid sequence of SEQ ID NO: 125, and the VL1 is at least 90%, at least 95%, at least 96%, at least 97%, at least 98%, or at least 99% identical to the amino acid sequence of SEQ ID NO: 129. In some such antibodies, the VH1 is at least 90%, at least 95%, at least 96%, at least 97%, at least 98%, or at least 99% identical to the amino acid sequence SEQ ID NO: 133, and the VL1 is at least 90%, at least 95%, at least 96%, at least 97%, at least 98%, or at least 99% identical to the amino acid sequence of SEQ ID NO: 137. In some such antibodies, the VH1 is at least 90%, at least 95%, at least 96%, at least 97%, at least 98%, or at least 99% identical to the amino acid sequence of SEQ ID NO: 141, and the VL1 is at least 90%, at least 95%, at least 96%, at least 97%, at least 98%, or at least 99% identical to the amino acid sequence of SEQ ID NO: 145. In some such antibodies, the VH1 is at least 90%, at least 95%, at least 96%, at least 97%, at least 98%, or at least 99% identical to the amino acid sequence of SEQ ID NO: 149, and the VL1 is at least 90%, at least 95%, at least 96%, at least 97%, at least 98%, or at least 99% identical to the amino acid sequence of SEQ ID NO: 153. In some such antibodies, the VH1 is at least 90%, at least 95%, at least 96%, at least 97%, at least 98%, or at least 99% identical to the amino acid sequence of SEQ ID NO: 157, and the VL1 is at least 90%, at least 95%, at least 96%, at least 97%, at least 98%, or at least 99% identical to the amino acid sequence of SEQ ID NO: 159. In some such antibodies, the VH1 is at least 90%, at least 95%, at least 96%, at least 97%, at least 98%, or at least 99% identical to the amino acid sequence of SEQ ID NO: 161, and the VL1 is at least 90%, at least 95%, at least 96%, at least 97%, at least 98%, or at least 99% identical to the amino acid sequence of SEQ ID NO: 165. In some such antibodies, the VH1 is at least 90%, at least 95%, at least 96%, at least 97%, at least 98%, or at least 99% identical to the amino acid sequence of SEQ ID NO: 169, and the VL1 is at least 90%, at least 95%, at least 96%, at least 97%, at least 98%, or at least 99% identical to the amino acid sequence of SEQ ID NO: 173. In some such antibodies, the VH1 and the VL1 are at least 90%, at least 95%, at least 96%, at least 97%, at least 98%, or at least 99% identical to any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH1 and the VL1 are at least 90%, at least 95%, at least 96%, at least 97%, at least 98%, or at least 99% identical to any pair of anti-TL1A variable heavy domain and variable light domain in Tables 29 and 31-34.

In some such antibodies, the VH1 comprises the amino acid sequence of any one of SEQ ID NOS: 2241, 2253, 2259, 1674, and 1200, and the VL1 comprises the amino acid sequence of any one of SEQ ID NOS: 2243 and 1204. In some such antibodies, the VH1 comprises the amino acid sequence of SEQ ID NO: 2241, and the VL1 comprises the amino acid sequence of SEQ ID NO: 2243. In some such antibodies, the VH1 comprises the amino acid sequence of SEQ ID NO: 2253, and the VL1 comprises the amino acid sequence of SEQ ID NO: 2243. In some such antibodies, the VH1 comprises the amino acid sequence of SEQ ID NO: 2259, and the VL1 comprises the amino acid sequence of SEQ ID NO: 2243. In some such antibodies, the VH1 comprises the amino acid sequence of SEQ ID NO: 1674, and the VL1 comprises the amino acid sequence of SEQ ID NO: 1204. In some such antibodies, the VH1 comprises the amino acid sequence of SEQ ID NO: 1200, and the VL1 comprises the amino acid sequence of SEQ ID NO: 1204. In some such antibodies, the VH1 comprises the amino acid sequence of any one of SEQ ID NOS: 1427-1823 and 2316-2330, and the VL1 comprises the amino acid sequence of any one of SEQ ID NOS: 1824-1923. In some such antibodies, the VH1 comprises the amino acid sequence of any one of SEQ ID NOS: 1674, 1200, 1661, and 2323, and the VL1 comprises the amino acid sequence of SEQ ID NO: 1204. In some such antibodies, the VH1 comprises the amino acid sequence of SEQ ID NO: 1661, and the VL1 comprises the amino acid sequence of SEQ ID NO: 1204. In some such antibodies, the VH1 comprises the amino acid sequence of SEQ ID NO: 2323, and the VL1 comprises the amino acid sequence of SEQ ID NO: 1204. In some such antibodies, the VH1 comprises the amino acid sequence of SEQ ID NO: 101, and the VL1 comprises the amino acid sequence of SEQ ID NO: 105. In some such antibodies, the VH1 comprises the amino acid sequence of SEQ ID NO: 109, and the VL1 comprises the amino acid sequence of SEQ ID NO: 113. In some such antibodies, the VH1 comprises the amino acid sequence of SEQ ID NO: 117, and the VL1 comprises the amino acid sequence of SEQ ID NO: 121. In some such antibodies, the VH1 comprises the amino acid sequence of SEQ ID NO: 125, and the VL1 comprises the amino acid sequence of SEQ ID NO: 129. In some such antibodies, the VH1 comprises the amino acid sequence of SEQ ID NO: 133, and the VL1 comprises the amino acid sequence of SEQ ID NO: 137. In some such antibodies, the VH1 comprises the amino acid sequence of SEQ ID NO: 141, and the VL1 comprises the amino acid sequence of SEQ ID NO: 145. In some such antibodies, the VH1 comprises the amino acid sequence of SEQ ID NO: 149, and the VL1 comprises the amino acid sequence of SEQ ID NO: 153. In some such antibodies, the VH1 comprises the amino acid sequence of SEQ ID NO: 157, and the VL1 comprises the amino acid sequence of SEQ ID NO: 159. In some such antibodies, the VH1 comprises the amino acid sequence of SEQ ID NO: 161, and the VL1 comprises the amino acid sequence of SEQ ID NO: 165. In some such antibodies, the VH1 comprises the amino acid sequence of SEQ ID NO: 169, and the VL1 comprises the amino acid sequence of SEQ ID NO: 173. In some such antibodies, the VH1 and the VL1 comprise any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH1 and the VL1 comprise any pair of anti-TL1A variable heavy domain and variable light domain in Tables 29 and 31-34. In some embodiments, the VH1 or VL1 can comprise variants of any of the above in which an N-terminal glutamine or glutamate is replaced with pyroglutamate.

In some such antibodies, the VH2 and VL2 comprise a variable heavy domain comprising a vhCDR1, a vhCDR2, and a vhCDR3 contained within a variable heavy domain comprising the amino acid sequence of any one of SEQ ID NOS: 2233, 2235, and 179, and a variable light domain comprising a vlCDR1, a vlCDR2, and a vlCDR3 contained within a variable light domain comprising the amino acid sequence of any one of SEQ ID NOS: 2234, 2237, and 1248. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 2233, and a variable light domain comprising a vlCDR1, a vlCDR2, and a vlCDR3 contained within a variable light domain comprising the amino acid sequence of SEQ ID NO: 2234. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 2235, and a variable light domain comprising a vlCDR1, a vlCDR2, and a vlCDR3 contained within a variable light domain comprising the amino acid sequence of SEQ ID NO: 2237. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 179, and a variable light domain comprising a vlCDR1, a vlCDR2, and a vlCDR3 contained within a variable light domain comprising the amino acid sequence of SEQ ID NO: 1248. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 2113, and a variable light domain comprising a vlCDR1, a vlCDR2, and a vlCDR3 contained within a variable light domain comprising the amino acid sequence of SEQ ID NO: 2231. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 1967, and a variable light domain comprising a vlCDR1, a vlCDR2, and a vlCDR3 contained within a variable light domain comprising the amino acid sequence of SEQ ID NO: 1248. In some such antibodies, the VH2 and VL2 comprise a variable heavy domain comprising a vhCDR1, a vhCDR2, and a vhCDR3 and a variable light domain comprising a vlCDR1, a vlCDR2, and a vlCDR3 contained within a variable heavy domain and variable light domain pair in XENP53412, XENP53413, XENP53369, XENP53375, XENP53387, XENP53415, XENP53370, XENP53371, XENP53376, XENP53377, XENP53378, XENP53379, XENP53380, or XENP53414. In any of the above, optionally the CDRs are as defined by the Kabat, Chothia, Kabat+Chothia, AbM, contact, or IMGT definition.

In some such antibodies, the VH2 and VL2 comprise a variable heavy domain comprising a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182 or 2236, and a variable light domain comprising a vlCDR1 comprising the amino acid sequence of SEQ ID NO: 184, a vlCDR2 comprising the amino acid sequence of SEQ ID NO: 1249 or 2238, and a vlCDR3 comprising the amino acid sequence of SEQ ID NO: 1250. In some such antibodies, the VH2 and VL2 comprise a variable heavy domain comprising a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and a variable light domain comprising a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH2 and VL2 comprise a variable heavy domain comprising a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 2236, and a variable light domain comprising a vlCDR1 comprising the amino acid sequence of SEQ ID NO: 184, a vlCDR2 comprising the amino acid sequence of SEQ ID NO: 2238, and a vlCDR3 comprising the amino acid sequence of SEQ ID NO: 1250. In some such antibodies, the VH2 and VL2 comprise a variable heavy domain comprising a vhCDR1 comprising the amino acid sequence of SEQ ID NO: 2239, a vhCDR2 comprising the amino acid sequence of SEQ ID NO: 181, and a vhCDR3 comprising the amino acid sequence of SEQ ID NO: 182, and a variable light domain comprising a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250.

In some such antibodies, the VH2 is at least 90%, at least 95%, at least 96%, at least 97%, at least 98%, or at least 99% identical to the amino acid sequence of any one of SEQ ID NOS: 2233, 2235, and 179, and the VL2 is at least 90%, at least 95%, at least 96%, at least 97%, at least 98%, or at least 99% identical to the amino acid sequence of any one of SEQ ID NOS: 2234, 2237, and 1248. In some such antibodies, the VH2 is at least 90%, at least 95%, at least 96%, at least 97%, at least 98%, or at least 99% identical to the amino acid sequence of SEQ ID NO: 2233, and the VL2 is at least 90%, at least 95%, at least 96%, at least 97%, at least 98%, or at least 99% identical to the amino acid sequence of SEQ ID NO: 2234. In some such antibodies, the VH2 is at least 90%, at least 95%, at least 96%, at least 97%, at least 98%, or at least 99% identical to the amino acid sequence of SEQ ID NO: 2235, and the VL2 is at least 90%, at least 95%, at least 96%, at least 97%, at least 98%, or at least 99% identical to the amino acid sequence of SEQ ID NO: 2237. In some such antibodies, the VH2 is at least 90%, at least 95%, at least 96%, at least 97%, at least 98%, or at least 99% identical to the amino acid sequence of SEQ ID NO: 179, and the VL2 is at least 90%, at least 95%, at least 96%, at least 97%, at least 98%, or at least 99% identical to the amino acid sequence of SEQ ID NO: 1248. In some such antibodies, the VH2 is at least 90%, at least 95%, at least 96%, at least 97%, at least 98%, or at least 99% identical to the amino acid sequence of SEQ ID NO: 2113, and the VL2 is at least 90%, at least 95%, at least 96%, at least 97%, at least 98%, or at least 99% identical to the amino acid sequence of SEQ ID NO: 2231. In some such antibodies, the VH2 is at least 90%, at least 95%, at least 96%, at least 97%, at least 98%, or at least 99% identical to the amino acid sequence of SEQ ID NO: 1967, and the VL2 is at least 90%, at least 95%, at least 96%, at least 97%, at least 98%, or at least 99% identical to the amino acid sequence of SEQ ID NO: 1248. In some such antibodies, the VH2 is at least 90%, at least 95%, at least 96%, at least 97%, at least 98%, or at least 99% identical to the amino acid sequence of any one of SEQ ID NOS: 1927-2132, and the VL2 is at least 90%, at least 95%, at least 96%, at least 97%, at least 98%, or at least 99% identical to the amino acid sequence of any one of SEQ ID NOS: 2133-2232. In some such antibodies, the VH2 and the VL2 are at least 90%, at least 95%, at least 96%, at least 97%, at least 98%, or at least 99% identical to any pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH2 and the VL2 are at least 90%, at least 95%, at least 96%, at least 97%, at least 98%, or at least 99% identical to any pair of anti-IL23 variable heavy domain and variable light domain in Table 29 or 33.

In some such antibodies, the VH2 comprises the amino acid sequence of any one of SEQ ID NOS: 2233, 2235, and 179, and the VL2 comprises the amino acid sequence of any one of SEQ ID NOS: 2234, 2237, and 1248. In some such antibodies, the VH2 comprises the amino acid sequence of SEQ ID NO: 2233, and the VL2 comprises the amino acid sequence of SEQ ID NO: 2234. In some such antibodies, the VH2 comprises the amino acid sequence of SEQ ID NO: 2235, and the VL2 comprises the amino acid sequence of SEQ ID NO: 2237. In some such antibodies, the VH2 comprises the amino acid sequence of SEQ ID NO: 179, and the VL2 comprises the amino acid sequence of SEQ ID NO: 1248. In some such antibodies, the VH2 comprises the amino acid sequence of SEQ ID NO: 2113, and the VL2 comprises the amino acid sequence of SEQ ID NO: 2231. In some such antibodies, the VH2 comprises the amino acid sequence of SEQ ID NO: 1967, and the VL2 comprises the amino acid sequence of SEQ ID NO: 1248. In some such antibodies, the VH2 comprises the amino acid sequence of any one of SEQ ID NOS: 1927-2132, and the VL2 comprises the amino acid sequence of any one of SEQ ID NOS: 2133-2232. In some such antibodies, the VH2 and the VL2 comprise any pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH2 and the VL2 comprise any pair of anti-IL23 variable heavy domain and variable light domain in Table 29 or 33. In some such antibodies, the VH2 or VL2 can comprise variants of any of the above in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of any one of SEQ ID NOS: 2241, 2253, 2259, 1674, and 1200, (ii) the VL1 comprises a vlCDR1, a vlCDR2, and a vlCDR3 contained within a variable light domain comprising the amino acid sequence of any one of SEQ ID NOS: 2243 and 1204, (iii) the VH2 comprises a vhCDR1, a vhCDR2, and a vhCDR3 contained within a variable heavy domain comprising the amino acid sequence of any one of SEQ ID NOS: 2233, 2235, and 179, and (iv) the VL2 comprises a vlCDR1, a vlCDR2, and a vlCDR3 contained within a variable light domain comprising the amino acid sequence of any one of SEQ ID NOS: 2234, 2237, and 1248, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102 or 1201, a vhCDR2 comprising the amino acid sequence of SEQ ID NO: 1924 or 1202, and a vhCDR3 comprising the amino acid sequence of SEQ ID NO: 1203 or 104, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182 or 2236, and (iv) the VL2 comprises a vlCDR1 comprising the amino acid sequence of SEQ ID NO: 184, a vlCDR2 comprising the amino acid sequence of SEQ ID NO: 1249 or 2238, and a vlCDR3 comprising the amino acid sequence of SEQ ID NO: 1250. In some such antibodies, the VH1 comprises the amino acid sequence of any one of SEQ ID NOS: 2241, 2253, 2259, 1674, and 1200, (ii) the VL1 comprises the amino acid sequence of any one of SEQ ID NOS: 2243 and 1204, (iii) the VH2 comprises the amino acid sequence of any one of SEQ ID NOS: 2233, 2235, and 179, and (iv) the VL2 comprises the amino acid sequence of any one of SEQ ID NOS: 2234, 2237, and 1248, or variants thereof in which an N-terminal glutamine or glutamate is replaced with pyroglutamate. In some such antibodies, the bispecific antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the bispecific antibody specifically binds human TL1A with a K_{D} of less than about 3E-11 M, less than about 2.9E-11 M, less than about 2.8E-11 M, or less than about 2.7E-11 M. In some such antibodies, the bispecific antibody inhibits TL1A activity in a TL1A luciferase reporter assay with an IC₅₀ of less than about 4.5 nM. In some such antibodies, the bispecific antibody specifically binds human IL23 with a K_{D} of less than about 1.1E-10 M as measured by surface plasmon resonance or Biacore at 25° C., optionally wherein the bispecific antibody specifically binds human IL23 with a K_{D} of less than about 6E-11 M, less than about 5.9E-11 M, less than about 5.8E-11 M, or less than about 5.7E-11 M. In some such antibodies, the bispecific antibody inhibits IL23 activity in an IL23 luciferase reporter assay with an IC₅₀ of less than about 65 pM, less than about 64 pM, or less than about 63 pM, optionally wherein the bispecific antibody inhibits TL23 activity with an IC₅₀ of less than about 60 pM, less than about 59 pM, less than about 58 pM, less than about 57 pM, or less than about 56 pM.

In some such antibodies, the VH1 comprises a vhCDR1, a vhCDR2, and a vhCDR3 contained within a variable heavy domain comprising the amino acid sequence of SEQ ID NO: 2241, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence of SEQ ID NO: 2241, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234, or variants thereof in which an N-terminal glutamine or glutamate is replaced with pyroglutamate. In some such antibodies, the bispecific antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the bispecific antibody specifically binds human TL1A with a K_{D} of less than about 3E-11 M, less than about 2.9E-11 M, less than about 2.8E-11 M, or less than about 2.7E-11 M. In some such antibodies, the bispecific antibody inhibits TL1A activity in a TL1A luciferase reporter assay with an IC₅₀ of less than about 4.5 nM. In some such antibodies, the bispecific antibody specifically binds human IL23 with a K_{D} of less than about 1.1E-10 M as measured by surface plasmon resonance or Biacore at 25° C., optionally wherein the bispecific antibody specifically binds human IL23 with a K_{D} of less than about 6E-11 M, less than about 5.9E-11 M, less than about 5.8E-11 M, or less than about 5.7E-11 M. In some such antibodies, the bispecific antibody inhibits IL23 activity in an IL23 luciferase reporter assay with an IC₅₀ of less than about 65 pM, less than about 64 pM, or less than about 63 pM, optionally wherein the bispecific antibody inhibits IL23 activity with an IC₅₀ of less than about 60 pM, less than about 59 pM, less than about 58 pM, less than about 57 pM, or less than about 56 pM.

In some such antibodies, the VH1 comprises a vhCDR1, a vhCDR2, and a vhCDR3 contained within a variable heavy domain comprising the amino acid sequence of SEQ ID NO: 2241, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2235, and (iv) the VL2 comprises a vlCDR1, a vlCDR2, and a vlCDR3 contained within a variable light domain comprising the amino acid sequence of SEQ ID NO: 2237. In some such antibodies, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 2236, and (iv) the VL2 comprises a vlCDR1 comprising the amino acid sequence of SEQ ID NO: 184, a vlCDR2 comprising the amino acid sequence of SEQ ID NO: 2238, and a vlCDR3 comprising the amino acid sequence of SEQ ID NO: 1250. In some such antibodies, the VH1 comprises the amino acid sequence of SEQ ID NO: 2241, (ii) the VL1 comprises the amino acid sequence of SEQ ID NO: 2243, (iii) the VH2 comprises the amino acid sequence of SEQ ID NO: 2235, and (iv) the VL2 comprises the amino acid sequence of SEQ ID NO: 2237, or variants thereof in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of SEQ ID NO: 2253, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence of SEQ ID NO: 2253, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234, or variants thereof in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of SEQ ID NO: 2259, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 104, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence of SEQ ID NO: 2259, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234, or variants thereof in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of SEQ ID NO: 1674, (ii) the VL1 comprises a vlCDR1, a vlCDR2, and a vlCDR3 contained within a variable light domain comprising the amino acid sequence of SEQ ID NO: 1204, (iii) the VH2 comprises a vhCDR1, a vhCDR2, and a vhCDR3 contained within a variable heavy domain comprising the amino acid sequence of SEQ ID NO: 179, and (iv) the VL2 comprises a vlCDR1, a vlCDR2, and a vlCDR3 contained within a variable light domain comprising the amino acid sequence of SEQ ID NO: 1248, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence of SEQ ID NO: 1674, (ii) the VL1 comprises the amino acid sequence of SEQ ID NO: 1204, (iii) the VH2 comprises the amino acid sequence of SEQ ID NO: 179, and (iv) the VL2 comprises the amino acid sequence of SEQ ID NO: 1248, or variants thereof in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of SEQ ID NO: 1200, (ii) the VL1 comprises a vlCDR1, a vlCDR2, and a vlCDR3 contained within a variable light domain comprising the amino acid sequence of SEQ ID NO: 1204, (iii) the VH2 comprises a vhCDR1, a vhCDR2, and a vhCDR3 contained within a variable heavy domain comprising the amino acid sequence of SEQ ID NO: 179, and (iv) the VL2 comprises a vlCDR1, a vlCDR2, and a vlCDR3 contained within a variable light domain comprising the amino acid sequence of SEQ ID NO: 1248, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence SEQ ID NO: 1200, (ii) the VL1 comprises the amino acid sequence of SEQ ID NO: 1204, (iii) the VH2 comprises the amino acid sequence of SEQ ID NO: 179, and (iv) the VL2 comprises the amino acid sequence of SEQ ID NO: 1248, or variants thereof in which an N-terminal glutamine or glutamate is replaced with pyroglutamate.

In some such antibodies, the bispecific antibody comprises (i) a first heavy chain comprising the VH1 and a first heavy chain constant domain, (ii) a first light chain comprising the VL1 and a first light chain constant domain, (iii) a second heavy chain comprising the VH2 and a second heavy chain constant domain, and (iv) a second light chain comprising the VL2 and a second light chain constant domain. In some such antibodies, the first heavy chain constant domain and the second heavy chain constant domain comprise a set of heterodimerization skew variants. In some such antibodies, the first heavy chain constant domain and the second heavy chain constant domain each comprise one or more ablation variants. In some such antibodies, the first heavy chain constant domain or the second heavy chain constant domain comprises one or more pI variants. In some such antibodies, the first heavy chain constant domain comprises amino acid substitutions E233P/L234V/L235A/G236del/S267K and/or the second heavy chain constant domain comprises amino acid substitutions E233P/L234V/L235A/G236del/S267K, wherein numbering is according to EU numbering. In some such antibodies, the first heavy chain constant domain comprises amino acid substitutions M428L/N434S and/or the second heavy chain constant domain comprises amino acid substitutions M428L/N434S, wherein numbering is according to EU numbering. In some such antibodies, the first heavy chain constant domain comprises amino acid substitutions E233P/L234V/L235A/G236del/S267K and M428L/N434S and/or the second heavy chain constant domain comprises amino acid substitutions E233P/L234V/L235A/G236del/S267K and M428L/N434S, wherein numbering is according to EU numbering. In some such antibodies, the first heavy chain constant domain or the second heavy chain constant domain comprises amino acid substitutions L368D/K370S, wherein numbering is according to EU numbering. In some such antibodies, the first heavy chain constant domain or the second heavy chain constant domain comprises amino acid substitutions N208D/Q295E/N384D/Q418E/N421D, wherein numbering is according to EU numbering. In some such antibodies, the first heavy chain constant domain or the second heavy chain constant domain comprises amino acid substitutions E233P/L234V/L235A/G236del/S267K, M428L/N434S, L368D/K370S, and N208D/Q295E/N384D/Q418E/N421D, wherein numbering is according to EU numbering. In some such antibodies, the first heavy chain constant domain or the second heavy chain constant domain comprises amino acid substitutions E357Q/S364K, wherein numbering is according to EU numbering. In some such antibodies, the first heavy chain constant domain comprises amino acid substitutions L368D/K370S, and the second heavy chain constant domain comprises amino acid substitutions E357Q/S364K, wherein numbering is according to EU numbering. In some such antibodies, the first heavy chain constant domain or the second heavy chain constant domain comprises amino acid substitutions E233P/L234V/L235A/G236del/S267K, M428L/N434S, and E357Q/S364K, wherein numbering is according to EU numbering. In some such antibodies, the first heavy chain constant domain comprises amino acid substitutions E233P/L234V/L235A/G236del/S267K, M428L/N434S, L368D/K370S, and N208D/Q295E/N384D/Q418E/N421D, wherein numbering is according to EU numbering, and the second heavy chain constant domain comprises amino acid substitutions E233P/L234V/L235A/G236del/S267K, M428L/N434S, and E357Q/S364K, wherein numbering is according to EU numbering.

In some such antibodies, the first heavy chain constant domain is of isotype IgG1, and the second heavy chain constant domain is of isotype IgG1. In some such antibodies, the bispecific antibody is a chimeric or humanized bispecific antibody.

In some such antibodies, the bispecific antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the bispecific antibody specifically binds human TL1A with a K_{D} of less than about 3E-11 M, less than about 2.9E-11 M, less than about 2.8E-11 M, or less than about 2.7E-11 M. In some such antibodies, the bispecific antibody inhibits TL1A activity in a TL1A luciferase reporter assay with an IC₅₀ of less than about 4.5 nM. In some such antibodies, the bispecific antibody specifically binds human IL23 with a K_{D} of less than about 1.1E-10 M as measured by surface plasmon resonance or Biacore at 25° C., optionally wherein the bispecific antibody specifically binds human IL23 with a K_{D} of less than about 6E-11 M, less than about 5.9E-11 M, less than about 5.8E-11 M, or less than about 5.7E-11 M. In some such antibodies, the bispecific antibody inhibits IL23 activity in an IL23 luciferase reporter assay with an IC₅₀ of less than about 65 pM, less than about 64 pM, or less than about 63 pM, optionally wherein the bispecific antibody inhibits IL23 activity with an IC₅₀ of less than about 60 pM, less than about 59 pM, less than about 58 pM, less than about 57 pM, or less than about 56 pM.

In another aspect, provided are bispecific or heterodimeric antibodies comprising (a) a first monomer comprising a first heavy chain comprising a VH1-CH1-hinge-CH2-CH3, wherein VH1 is a first variable heavy domain, CH1-hinge-CH2-CH3 is a first heavy chain constant domain, and CH2-CH3 is a first Fc domain; (b) a second monomer comprising a second heavy chain comprising a VH2-CH1-hinge-CH2-CH3, wherein VH2 is a second variable domain, CH1-hinge-CH2-CH3 is a second heavy chain constant domain, and CH2-CH3 is a second Fc domain; (c) a first light chain comprising a VL1-CL, wherein VL1 is a first variable light domain, and CL is a first light chain constant domain; and (d) a second light chain comprising a VL2-CL, wherein VL2 is a second variable light domain, and CL is a second light chain constant domain, wherein the VH1 and VL1 together form a first antigen-binding domain (ABD), and the VH2 and the VL2 together form a second ABD, wherein the first ABD is a TL1A-binding domain and the second ABD is an IL23-binding domain, or wherein the first ABD is an IL23-binding domain and the second ABD is a TL1A-binding domain, and wherein: (i) the first heavy chain constant domain and the first light chain constant domain comprise a set of electrostatic steering variants at positions 213 and 218 of the first heavy chain constant domain according to EU numbering and positions 122 and 123 of the first light chain constant domain according to Kabat numbering, or a set of steric skew variants at position 141 of the first heavy chain constant domain according to EU numbering and position 118 of the first light chain constant domain according to Kabat numbering, at position 141 of the first heavy chain constant domain according to EU numbering and position 116 of the first light chain constant domain according to Kabat numbering, or at position 147 of the first heavy chain constant domain according to EU numbering and position 131 of the first light chain constant domain according to Kabat numbering; and/or (ii) the VH1 and the VL1 comprise a set of electrostatic steering variants at position 39 of VH1 and position 38 of VL1 according to Kabat numbering; and/or (iii) the second heavy chain constant domain and the second light chain constant domain comprise a set of electrostatic steering variants at positions 213 and 218 of the second heavy chain constant domain according to EU numbering and positions 122 and 123 of the second light chain constant domain according to Kabat numbering, or a set of steric skew variants at position 141 of the second heavy chain constant domain according to EU numbering and position 118 of the second light chain constant domain according to Kabat numbering, at position 141 of the second heavy chain constant domain according to EU numbering and position 116 of the second light chain constant domain according to Kabat numbering, or at position 147 of the second heavy chain constant domain according to EU numbering and position 131 of the second light chain constant domain according to Kabat numbering; and/or (iv) the VH2 and the VL2 comprise a set of electrostatic steering variants at position 39 of VH2 and position 38 of VL2 according to Kabat numbering. For example, any of the above antibodies can be in this format.

In some such antibodies, the first heavy chain constant domain and the first light chain constant domain comprise a set of electrostatic steering variants as depicted in FIG. 4 or Table 27 or a set of steric skew variants as depicted in FIG. 5 or Table 28. In some such antibodies, the VH1 and the VL1 comprise a set of electrostatic steering variants as depicted in FIG. 6 or Table 18 or 19. In some such antibodies, the second heavy chain constant domain and the second light chain constant domain comprise a set of electrostatic steering variants as depicted in FIG. 4 or Table 27 or a set of steric skew variants as depicted in FIG. 5 or Table 28. In some such antibodies, the VH2 and the VL2 comprise a set of electrostatic steering variants as depicted in FIG. 6 or Table 18 or 19.

In some such antibodies, the VH1 comprises amino acid substitution Q39E and the VL1 comprises amino acid substitution Q38K according to Kabat numbering. In some such antibodies, the VH2 comprises amino acid substitution Q39K and the VL2 comprises amino acid substitution Q38E according to Kabat numbering. In some such antibodies, the VH1 comprises amino acid substitution Q39E, the VL1 comprises amino acid substitution Q38K, the VH2 comprises amino acid substitution Q39K, and the VL2 comprises amino acid substitution Q38E according to Kabat numbering.

In some such antibodies, the first heavy chain constant domain comprises amino acid substitutions K213E/K218D according to EU numbering, and the first light chain constant domain comprises amino acid substitutions D122K/E123K according to Kabat numbering. In some such antibodies, the second heavy chain constant domain comprises amino acid substitution A141F according to EU numbering, and the second light chain constant domain comprises amino acid substitution F118A according to Kabat numbering. In some such antibodies, (a) the VH1 comprises amino acid substitution Q39E, the VL1 comprises amino acid substitution Q38K, the VH2 comprises amino acid substitution Q39K, and the VL2 comprises amino acid substitution Q38E according to Kabat numbering; (b) the first heavy chain constant domain comprises amino acid substitutions K213E/K218D according to EU numbering, and the first light chain constant domain comprises amino acid substitutions D122K/E123K according to Kabat numbering; and (c) the second heavy chain constant domain comprises amino acid substitution A141F according to EU numbering, and the second light chain constant domain comprises amino acid substitution F118A according to Kabat numbering.

In some such antibodies, the first ABD is a TL1A-binding domain and the second ABD is an IL23-binding domain.

In some such antibodies, the VH1 and VL1 comprise a variable heavy domain comprising a vhCDR1, a vhCDR2, and a vhCDR3 contained within a variable heavy domain comprising the amino acid sequence of any one of SEQ ID NOS: 2241, 2253, 2259, 1674, and 1200, and a variable light domain comprising a vlCDR1, a vlCDR2, and a vlCDR3 contained within a variable light domain comprising the amino acid sequence of any one of SEQ ID NOS: 2243 and 1204. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 2241, and a variable light domain comprising a vlCDR1, a vlCDR2, and a vlCDR3 contained within a variable light domain comprising the amino acid sequence of SEQ ID NO: 2243. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 2253, and a variable light domain comprising a vlCDR1, a vlCDR2, and a vlCDR3 contained within a variable light domain comprising the amino acid sequence of SEQ ID NO: 2243. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 2259, and a variable light domain comprising a vlCDR1, a vlCDR2, and a vlCDR3 contained within a variable light domain comprising the amino acid sequence of SEQ ID NO: 2243. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 1674, and a variable light domain comprising a vlCDR1, a vlCDR2, and a vlCDR3 contained within a variable light domain comprising the amino acid sequence of SEQ ID NO: 1204. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 1200, and a variable light domain comprising a vlCDR1, a vlCDR2, and a vlCDR3 contained within a variable light domain comprising the amino acid sequence of SEQ ID NO: 1204. In some such antibodies, the VH1 and VL1 comprise a variable heavy domain comprising a vhCDR1, a vhCDR2, and a vhCDR3 contained within a variable heavy domain comprising the amino acid sequence of any one of SEQ ID NOS: 1427-1823 and 2316-2330, and a variable light domain comprising a vlCDR1, a vlCDR2, and a vlCDR3 contained within a variable light domain comprising the amino acid sequence of any one of SEQ ID NOS: 1824-1923. In some such antibodies, the VH1 and VL1 comprise a variable heavy domain comprising a vhCDR1, a vhCDR2, and a vhCDR3 contained within a variable heavy domain comprising the amino acid sequence of any one of SEQ ID NOS: 1674, 1200, 1661, and 2323, and a variable light domain comprising a vlCDR1, a vlCDR2, and a vlCDR3 contained within a variable light domain comprising the amino acid sequence of SEQ ID NO: 1204. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 1661, and a variable light domain comprising a vlCDR1, a vlCDR2, and a vlCDR3 contained within a variable light domain comprising the amino acid sequence of SEQ ID NO: 1204. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 2323, and a variable light domain comprising a vlCDR1, a vlCDR2, and a vlCDR3 contained within a variable light domain comprising the amino acid sequence of SEQ ID NO: 1204. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 101, and a variable light domain comprising a vlCDR1, a vlCDR2, and a vlCDR3 contained within a variable light domain comprising the amino acid sequence of SEQ ID NO: 105. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 109, and a variable light domain comprising a vlCDR1, a vlCDR2, and a vlCDR3 contained within a variable light domain comprising the amino acid sequence of SEQ ID NO: 113. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 117, and a variable light domain comprising a vlCDR1, a vlCDR2, and a vlCDR3 contained within a variable light domain comprising the amino acid sequence of SEQ ID NO: 121. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 125, and a variable light domain comprising a vlCDR1, a vlCDR2, and a vlCDR3 contained within a variable light domain comprising the amino acid sequence of SEQ ID NO: 129. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 133, and a variable light domain comprising a vlCDR1, a vlCDR2, and a vlCDR3 contained within a variable light domain comprising the amino acid sequence of SEQ ID NO: 137. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 141, and a variable light domain comprising a vlCDR1, a vlCDR2, and a vlCDR3 contained within a variable light domain comprising the amino acid sequence of SEQ ID NO: 145. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 149, and a variable light domain comprising a vlCDR1, a vlCDR2, and a vlCDR3 contained within a variable light domain comprising the amino acid sequence of SEQ ID NO: 153. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 157, and a variable light domain comprising a vlCDR1, a vlCDR2, and a vlCDR3 contained within a variable light domain comprising the amino acid sequence of SEQ ID NO: 159. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 161, and a variable light domain comprising a vlCDR1, a vlCDR2, and a vlCDR3 contained within a variable light domain comprising the amino acid sequence of SEQ ID NO: 165. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 169, and a variable light domain comprising a vlCDR1, a vlCDR2, and a vlCDR3 contained within a variable light domain comprising the amino acid sequence of SEQ ID NO: 173. In some such antibodies, the VH1 and VL1 comprise a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH1 and VL1 comprise a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in Tables 26 and 29-34. Optionally, the CDRs in any of the above antibodies are as defined by the Kabat, Chothia, Kabat+Chothia, AbM, contact, or IMGT definition.

In some such antibodies, the VH1 and VL1 comprise a variable heavy domain comprising a vhCDR1 comprising the amino acid sequence of SEQ ID NO: 102 or 1201, a vhCDR2 comprising the amino acid sequence of SEQ ID NO: 1924 or 1202, and a vhCDR3 comprising the amino acid sequence of SEQ ID NO: 1203 or 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and VL1 comprise a variable heavy domain comprising a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and VL1 comprise a variable heavy domain comprising a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and VL1 comprise a variable heavy domain comprising a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and VL1 comprise a variable heavy domain comprising a vhCDR1 comprising the amino acid sequence of SEQ ID NO: 102, a vhCDR2 comprising the amino acid sequence of SEQ ID NO: 1926, and a vhCDR3 comprising the amino acid sequence of SEQ ID NO: 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and VL1 comprise a variable heavy domain comprising a vhCDR1 comprising the amino acid sequence of SEQ ID NO: 102, a vhCDR2 comprising the amino acid sequence of SEQ ID NO: 103, and a vhCDR3 comprising the amino acid sequence of SEQ ID NO: 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and VL1 comprise a variable heavy domain comprising a vhCDR1 comprising the amino acid sequence of SEQ ID NO: 110, a vhCDR2 comprising the amino acid sequence of SEQ ID NO: 111, and a vhCDR3 comprising the amino acid sequence of SEQ ID NO: 112, and a variable light domain comprising a vlCDR1 comprising the amino acid sequence of SEQ ID NO: 114, a vlCDR2 comprising the amino acid sequence of SEQ ID NO: 115, and a vlCDR3 comprising the amino acid sequence of SEQ ID NO: 116. In some such antibodies, the VH1 and VL1 comprise a variable heavy domain comprising a vhCDR1 comprising the amino acid sequence of SEQ ID NO: 118, a vhCDR2 comprising the amino acid sequence of SEQ ID NO: 119, and a vhCDR3 comprising the amino acid sequence of SEQ ID NO: 120, and a variable light domain comprising a vlCDR1 comprising the amino acid sequence of SEQ ID NO: 122, a vlCDR2 comprising the amino acid sequence of SEQ ID NO: 123, and a vlCDR3 comprising the amino acid sequence of SEQ ID NO: 124. In some such antibodies, the VH1 and VL1 comprise a variable heavy domain comprising a vhCDR1 comprising the amino acid sequence of SEQ ID NO: 126, a vhCDR2 comprising the amino acid sequence of SEQ ID NO: 127, and a vhCDR3 comprising the amino acid sequence of SEQ ID NO: 128, and a variable light domain comprising a vlCDR1 comprising the amino acid sequence of SEQ ID NO: 130, a vlCDR2 comprising the amino acid sequence of SEQ ID NO: 131, and a vlCDR3 comprising the amino acid sequence of the amino acid sequence of SEQ ID NO: 132. In some such antibodies, the VH1 and VL1 comprise a variable heavy domain comprising a vhCDR1 comprising the amino acid sequence of SEQ ID NO: 134, a vhCDR2 comprising the amino acid sequence of SEQ ID NO: 135, and a vhCDR3 comprising the amino acid sequence of SEQ ID NO: 136, and a variable light domain comprising a vlCDR1 comprising the amino acid sequence of SEQ ID NO: 138, a vlCDR2 comprising the amino acid sequence of SEQ ID NO: 139, and a vlCDR3 comprising the amino acid sequence of the amino acid sequence of SEQ ID NO: 140. In some such antibodies, the VH1 and VL1 comprise a variable heavy domain comprising a vhCDR1 comprising the amino acid sequence of SEQ ID NO: 142, a vhCDR2 comprising the amino acid sequence of SEQ ID NO: 143, and a vhCDR3 comprising the amino acid sequence of SEQ ID NO: 144, and a variable light domain comprising a vlCDR1 comprising the amino acid sequence of SEQ ID NO: 146, a vlCDR2 comprising the amino acid sequence of SEQ ID NO: 147, and a vlCDR3 comprising the amino acid sequence of SEQ ID NO: 148. In some such antibodies, the VH1 and VL1 comprise a variable heavy domain comprising a vhCDR1 comprising the amino acid sequence of SEQ ID NO: 150, a vhCDR2 comprising the amino acid sequence of SEQ ID NO: 151, and a vhCDR3 comprising the amino acid sequence of SEQ ID NO: 152, and a variable light domain comprising a vlCDR1 comprising the amino acid sequence of SEQ ID NO: 154, a vlCDR2 comprising the amino acid sequence of SEQ ID NO: 155, and a vlCDR3 comprising the amino acid sequence of SEQ ID NO: 156. In some such antibodies, the VH1 and VL1 comprise a variable heavy domain comprising a vhCDR1 comprising the amino acid sequence of SEQ ID NO: 118, a vhCDR2 comprising the amino acid sequence of SEQ ID NO: 158, and a vhCDR3 comprising the amino acid sequence of SEQ ID NO: 120, and a variable light domain comprising a vlCDR1 comprising the amino acid sequence of SEQ ID NO: 122, a vlCDR2 comprising the amino acid sequence of SEQ ID NO: 123, and a vlCDR3 comprising the amino acid sequence of SEQ ID NO: 160. In some such antibodies, the VH1 and VL1 comprise a variable heavy domain comprising a vhCDR1 comprising the amino acid sequence of SEQ ID NO: 162, a vhCDR2 comprising the amino acid sequence of SEQ ID NO: 163, and a vhCDR3 comprising the amino acid sequence of SEQ ID NO: 164, and a variable light domain comprising a vlCDR1 comprising the amino acid sequence of SEQ ID NO: 166, a vlCDR2 comprising the amino acid sequence of SEQ ID NO: 167, and a vlCDR3 comprising the amino acid sequence of SEQ ID NO: 168. In some such antibodies, the VH1 and VL1 comprise a variable heavy domain comprising a vhCDR1 comprising the amino acid sequence of SEQ ID NO: 170, a vhCDR2 comprising the amino acid sequence of SEQ ID NO: 171, and a vhCDR3 comprising the amino acid sequence of SEQ ID NO: 172, and a variable light domain comprising a vlCDR1 comprising the amino acid sequence of SEQ ID NO: 174, a vlCDR2 comprising the amino acid sequence of SEQ ID NO: 175, and a vlCDR3 comprising the amino acid sequence of SEQ ID NO: 176.

In some such antibodies, the VH1 is at least 90%, at least 95%, at least 96%, at least 97%, at least 98%, or at least 99% identical to the amino acid sequence of any one of SEQ ID NOS: 2241, 2253, 2259, 1674, and 1200, and the VL1 is at least 90%, at least 95%, at least 96%, at least 97%, at least 98%, or at least 99% identical to the amino acid sequence of any one of SEQ ID NOS: 2243 and 1204. In some such antibodies, the VH1 is at least 90%, at least 95%, at least 96%, at least 97%, at least 98%, or at least 99% identical to the amino acid sequence of SEQ ID NO: 2241, and the VL1 is at least 90%, at least 95%, at least 96%, at least 97%, at least 98%, or at least 99% identical to the amino acid sequence of SEQ ID NO: 2243. In some such antibodies, the VH1 is at least 90%, at least 95%, at least 96%, at least 97%, at least 98%, or at least 99% identical to the amino acid sequence of SEQ ID NO: 2253, and the VL1 is at least 90%, at least 95%, at least 96%, at least 97%, at least 98%, or at least 99% identical to the amino acid sequence of SEQ ID NO: 2243. In some such antibodies, the VH1 is at least 90%, at least 95%, at least 96%, at least 97%, at least 98%, or at least 99% identical to the amino acid sequence of SEQ ID NO: 2259, and the VL1 is at least 90%, at least 95%, at least 96%, at least 97%, at least 98%, or at least 99% identical to the amino acid sequence of SEQ ID NO: 2243. In some such antibodies, the VH1 is at least 90%, at least 95%, at least 96%, at least 97%, at least 98%, or at least 99% identical to the amino acid sequence of SEQ ID NO: 1674, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of SEQ ID NO: 1200, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of any one of SEQ ID NOS: 1427-1823 and 2316-2330, and the VL1 is at least 90%, at least 95%, at least 96%, at least 97%, at least 98%, or at least 99% identical to the amino acid sequence of any one of SEQ ID NOS: 1824-1923. In some such antibodies, the VH1 is at least 90%, at least 95%, at least 96%, at least 97%, at least 98%, or at least 99% identical to the amino acid sequence of any one of SEQ ID NOS: 1674, 1200, 1661, and 2323, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of SEQ ID NO: 1661, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of SEQ ID NO: 2323, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of SEQ ID NO: 101, and the VL1 is at least 90%, at least 95%, at least 96%, at least 97%, at least 98%, or at least 99% identical to the amino acid sequence of SEQ ID NO: 105. In some such antibodies, the VH1 is at least 90%, at least 95%, at least 96%, at least 97%, at least 98%, or at least 99% identical to the amino acid sequence of SEQ ID NO: 109, and the VL1 is at least 90%, at least 95%, at least 96%, at least 97%, at least 98%, or at least 99% identical to the amino acid sequence of SEQ ID NO: 113. In some such antibodies, the VH1 is at least 90%, at least 95%, at least 96%, at least 97%, at least 98%, or at least 99% identical to the amino acid sequence of SEQ ID NO: 117, and the VL1 is at least 90%, at least 95%, at least 96%, at least 97%, at least 98%, or at least 99% identical to the amino acid sequence of SEQ ID NO: 121. In some such antibodies, the VH1 is at least 90%, at least 95%, at least 96%, at least 97%, at least 98%, or at least 99% identical to the amino acid sequence of SEQ ID NO: 125, and the VL1 is at least 90%, at least 95%, at least 96%, at least 97%, at least 98%, or at least 99% identical to the amino acid sequence of SEQ ID NO: 129. In some such antibodies, the VH1 is at least 90%, at least 95%, at least 96%, at least 97%, at least 98%, or at least 99% identical to the amino acid sequence SEQ ID NO: 133, and the VL1 is at least 90%, at least 95%, at least 96%, at least 97%, at least 98%, or at least 99% identical to the amino acid sequence of SEQ ID NO: 137. In some such antibodies, the VH1 is at least 90%, at least 95%, at least 96%, at least 97%, at least 98%, or at least 99% identical to the amino acid sequence of SEQ ID NO: 141, and the VL1 is at least 90%, at least 95%, at least 96%, at least 97%, at least 98%, or at least 99% identical to the amino acid sequence of SEQ ID NO: 145. In some such antibodies, the VH1 is at least 90%, at least 95%, at least 96%, at least 97%, at least 98%, or at least 99% identical to the amino acid sequence of SEQ ID NO: 149, and the VL1 is at least 90%, at least 95%, at least 96%, at least 97%, at least 98%, or at least 99% identical to the amino acid sequence of SEQ ID NO: 153. In some such antibodies, the VH1 is at least 90%, at least 95%, at least 96%, at least 97%, at least 98%, or at least 99% identical to the amino acid sequence of SEQ ID NO: 157, and the VL1 is at least 90%, at least 95%, at least 96%, at least 97%, at least 98%, or at least 99% identical to the amino acid sequence of SEQ ID NO: 159. In some such antibodies, the VH1 is at least 90%, at least 95%, at least 96%, at least 97%, at least 98%, or at least 99% identical to the amino acid sequence of SEQ ID NO: 161, and the VL1 is at least 90%, at least 95%, at least 96%, at least 97%, at least 98%, or at least 99% identical to the amino acid sequence of SEQ ID NO: 165. In some such antibodies, the VH1 is at least 90%, at least 95%, at least 96%, at least 97%, at least 98%, or at least 99% identical to the amino acid sequence of SEQ ID NO: 169, and the VL1 is at least 90%, at least 95%, at least 96%, at least 97%, at least 98%, or at least 99% identical to the amino acid sequence of SEQ ID NO: 173. In some such antibodies, the VH1 and the VL1 are at least 90%, at least 95%, at least 96%, at least 97%, at least 98%, or at least 99% identical to any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH1 and the VL1 are at least 90%, at least 95%, at least 96%, at least 97%, at least 98%, or at least 99% identical to any pair of anti-TL1A variable heavy domain and variable light domain in Tables 26 and 29-34.

In some such antibodies, the VH1 comprises the amino acid sequence of any one of SEQ ID NOS: 2241, 2253, 2259, 1674, and 1200, and the VL1 comprises the amino acid sequence of any one of SEQ ID NOS: 2243 and 1204. In some such antibodies, the VH1 comprises the amino acid sequence of SEQ ID NO: 2241, and the VL1 comprises the amino acid sequence of SEQ ID NO: 2243. In some such antibodies, the VH1 comprises the amino acid sequence of SEQ ID NO: 2253, and the VL1 comprises the amino acid sequence of SEQ ID NO: 2243. In some such antibodies, the VH1 comprises the amino acid sequence of SEQ ID NO: 2259, and the VL1 comprises the amino acid sequence of SEQ ID NO: 2243. In some such antibodies, the VH1 comprises the amino acid sequence of SEQ ID NO: 1674, and the VL1 comprises the amino acid sequence of SEQ ID NO: 1204. In some such antibodies, the VH1 comprises the amino acid sequence of SEQ ID NO: 1200, and the VL1 comprises the amino acid sequence of SEQ ID NO: 1204. In some such antibodies, the VH1 comprises the amino acid sequence of any one of SEQ ID NOS: 1427-1823 and 2316-2330, and the VL1 comprises the amino acid sequence of any one of SEQ ID NOS: 1824-1923. In some such antibodies, the VH1 comprises the amino acid sequence of any one of SEQ ID NOS: 1674, 1200, 1661, and 2323, and the VL1 comprises the amino acid sequence of SEQ ID NO: 1204. In some such antibodies, the VH1 comprises the amino acid sequence of SEQ ID NO: 1661, and the VL1 comprises the amino acid sequence of SEQ ID NO: 1204. In some such antibodies, the VH1 comprises the amino acid sequence of SEQ ID NO: 2323, and the VL1 comprises the amino acid sequence of SEQ ID NO: 1204. In some such antibodies, the VH1 comprises the amino acid sequence of SEQ ID NO: 101, and the VL1 comprises the amino acid sequence of SEQ ID NO: 105. In some such antibodies, the VH1 comprises the amino acid sequence of SEQ ID NO: 109, and the VL1 comprises the amino acid sequence of SEQ ID NO: 113. In some such antibodies, the VH1 comprises the amino acid sequence of SEQ ID NO: 117, and the VL1 comprises the amino acid sequence of SEQ ID NO: 121. In some such antibodies, the VH1 comprises the amino acid sequence of SEQ ID NO: 125, and the VL1 comprises the amino acid sequence of SEQ ID NO: 129. In some such antibodies, the VH1 comprises the amino acid sequence of SEQ ID NO: 133, and the VL1 comprises the amino acid sequence of SEQ ID NO: 137. In some such antibodies, the VH1 comprises the amino acid sequence of SEQ ID NO: 141, and the VL1 comprises the amino acid sequence of SEQ ID NO: 145. In some such antibodies, the VH1 comprises the amino acid sequence of SEQ ID NO: 149, and the VL1 comprises the amino acid sequence of SEQ ID NO: 153. In some such antibodies, the VH1 comprises the amino acid sequence of SEQ ID NO: 157, and the VL1 comprises the amino acid sequence of SEQ ID NO: 159. In some such antibodies, the VH1 comprises the amino acid sequence of SEQ ID NO: 161, and the VL1 comprises the amino acid sequence of SEQ ID NO: 165. In some such antibodies, the VH1 comprises the amino acid sequence of SEQ ID NO: 169, and the VL1 comprises the amino acid sequence of SEQ ID NO: 173. In some such antibodies, the VH1 and the VL1 comprise any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH1 and the VL1 comprise any pair of anti-TL1A variable heavy domain and variable light domain in Tables 26 and 29-34. In some such antibodies, the VH1 or VL1 can comprise variants of any above the above in which an N-terminal glutamine or glutamate is replaced with pyroglutamate.

In some such antibodies, the VH2 and VL2 comprise a variable heavy domain comprising a vhCDR1, a vhCDR2, and a vhCDR3 contained within a variable heavy domain comprising the amino acid sequence of any one of SEQ ID NOS: 2233, 2235, and 179, and a variable light domain comprising a vlCDR1, a vlCDR2, and a vlCDR3 contained within a variable light domain comprising the amino acid sequence of any one of SEQ ID NOS: 2234, 2237, and 1248. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 2233, and a variable light domain comprising a vlCDR1, a vlCDR2, and a vlCDR3 contained within a variable light domain comprising the amino acid sequence of SEQ ID NO: 2234. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 2235, and a variable light domain comprising a vlCDR1, a vlCDR2, and a vlCDR3 contained within a variable light domain comprising the amino acid sequence of SEQ ID NO: 2237. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 179, and a variable light domain comprising a vlCDR1, a vlCDR2, and a vlCDR3 contained within a variable light domain comprising the amino acid sequence of SEQ ID NO: 1248. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 2113, and a variable light domain comprising a vlCDR1, a vlCDR2, and a vlCDR3 contained within a variable light domain comprising the amino acid sequence of SEQ ID NO: 2231. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 1967, and a variable light domain comprising a vlCDR1, a vlCDR2, and a vlCDR3 contained within a variable light domain comprising the amino acid sequence of SEQ ID NO: 1248. In some such antibodies, the VH2 and VL2 comprise a variable heavy domain comprising a vhCDR1, a vhCDR2, and a vhCDR3 contained within a variable heavy domain comprising the amino acid sequence of any one of SEQ ID NOS: 1927-2132, and a variable light domain comprising a vlCDR1, a vlCDR2, and a vlCDR3 contained within a variable light domain comprising the amino acid sequence of any one of SEQ ID NOS: 2133-2232. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 179, and a variable light domain comprising a vlCDR1, a vlCDR2, and a vlCDR3 contained within a variable light domain comprising the amino acid sequence of SEQ ID NO: 183. In some such antibodies, the VH2 and VL2 comprise a variable heavy domain comprising a vhCDR1, a vhCDR2, and a vhCDR3 and a variable light domain comprising a vlCDR1, a vlCDR2, and a vlCDR3 contained within any pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH2 and VL2 comprise a variable heavy domain comprising a vhCDR1, a vhCDR2, and a vhCDR3 and a variable light domain comprising a vlCDR1, a vlCDR2, and a vlCDR3 contained within any pair of anti-IL23 variable heavy domain and variable light domain in Tables 26 and 29-34. In some such antibodies, optionally the CDRs are as defined by the Kabat, Chothia, Kabat+Chothia, AbM, contact, or IMGT definition.

In some such antibodies, the VH2 and VL2 comprise a variable heavy domain comprising a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182 or 2236, and a variable light domain comprising a vlCDR1 comprising the amino acid sequence of SEQ ID NO: 184, a vlCDR2 comprising the amino acid sequence of SEQ ID NO: 1249 or 2238, and a vlCDR3 comprising the amino acid sequence of SEQ ID NO: 1250. In some such antibodies, the VH2 and VL2 comprise a variable heavy domain comprising a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and a variable light domain comprising a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH2 and VL2 comprise a variable heavy domain comprising a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 2236, and a variable light domain comprising a vlCDR1 comprising the amino acid sequence of SEQ ID NO: 184, a vlCDR2 comprising the amino acid sequence of SEQ ID NO: 2238, and a vlCDR3 comprising the amino acid sequence of SEQ ID NO: 1250. In some such antibodies, the VH2 and VL2 comprise a variable heavy domain comprising a vhCDR1 comprising the amino acid sequence of SEQ ID NO: 2239, a vhCDR2 comprising the amino acid sequence of SEQ ID NO: 181, and a vhCDR3 comprising the amino acid sequence of SEQ ID NO: 182, and a variable light domain comprising a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH2 and VL2 comprise a variable heavy domain comprising a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and a variable light domain comprising a vlCDR1 comprising the amino acid sequence of SEQ ID NO: 184, a vlCDR2 comprising the amino acid sequence of SEQ ID NO: 115, and a vlCDR3 comprising the amino acid sequence of SEQ ID NO: 185.

In some such antibodies, the VH2 is at least 90%, at least 95%, at least 96%, at least 97%, at least 98%, or at least 99% identical to the amino acid sequence of any one of SEQ ID NOS: 2233, 2235, and 179, and the VL2 is at least 90%, at least 95%, at least 96%, at least 97%, at least 98%, or at least 99% identical to the amino acid sequence of any one of SEQ ID NOS: 2234, 2237, and 1248. In some such antibodies, the VH2 is at least 90%, at least 95%, at least 96%, at least 97%, at least 98%, or at least 99% identical to the amino acid sequence of SEQ ID NO: 2233, and the VL2 is at least 90%, at least 95%, at least 96%, at least 97%, at least 98%, or at least 99% identical to the amino acid sequence of SEQ ID NO: 2234. In some such antibodies, the VH2 is at least 90%, at least 95%, at least 96%, at least 97%, at least 98%, or at least 99% identical to the amino acid sequence of SEQ ID NO: 2235, and the VL2 is at least 90%, at least 95%, at least 96%, at least 97%, at least 98%, or at least 99% identical to the amino acid sequence of SEQ ID NO: 2237. In some such antibodies, the VH2 is at least 90%, at least 95%, at least 96%, at least 97%, at least 98%, or at least 99% identical to the amino acid sequence of SEQ ID NO: 179, and the VL2 is at least 90%, at least 95%, at least 96%, at least 97%, at least 98%, or at least 99% identical to the amino acid sequence of SEQ ID NO: 1248. In some such antibodies, the VH2 is at least 90%, at least 95%, at least 96%, at least 97%, at least 98%, or at least 99% identical to the amino acid sequence of SEQ ID NO: 2113, and the VL2 is at least 90%, at least 95%, at least 96%, at least 97%, at least 98%, or at least 99% identical to the amino acid sequence of SEQ ID NO: 2231. In some such antibodies, the VH2 is at least 90%, at least 95%, at least 96%, at least 97%, at least 98%, or at least 99% identical to the amino acid sequence of SEQ ID NO: 1967, and the VL2 is at least 90%, at least 95%, at least 96%, at least 97%, at least 98%, or at least 99% identical to the amino acid sequence of SEQ ID NO: 1248. In some such antibodies, the VH2 is at least 90%, at least 95%, at least 96%, at least 97%, at least 98%, or at least 99% identical to the amino acid sequence of any one of SEQ ID NOS: 1927-2132, and the VL2 is at least 90%, at least 95%, at least 96%, at least 97%, at least 98%, or at least 99% identical to the amino acid sequence of any one of SEQ ID NOS: 2133-2232. In some such antibodies, the VH2 is at least 90%, at least 95%, at least 96%, at least 97%, at least 98%, or at least 99% identical to the amino acid sequence of SEQ ID NO: 179, the VL2 is at least 90%, at least 95%, at least 96%, at least 97%, at least 98%, or at least 99% identical to the amino acid sequence of SEQ ID NO: 183. In some such antibodies, the VH2 and the VL2 are at least 90%, at least 95%, at least 96%, at least 97%, at least 98%, or at least 99% identical to any pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH2 and the VL2 are at least 90%, at least 95%, at least 96%, at least 97%, at least 98%, or at least 99% identical to any pair of anti-IL23 variable heavy domain and variable light domain in Tables 26 and 29-34.

In some such antibodies, the VH2 comprises the amino acid sequence of any one of SEQ ID NOS: 2233, 2235, and 179, and the VL2 comprises the amino acid sequence of any one of SEQ ID NOS: 2234, 2237, and 1248. In some such antibodies, the VH2 comprises the amino acid sequence of SEQ ID NO: 2233, and the VL2 comprises the amino acid sequence of SEQ ID NO: 2234. In some such antibodies, the VH2 comprises the amino acid sequence of SEQ ID NO: 2235, and the VL2 comprises the amino acid sequence of SEQ ID NO: 2237. In some such antibodies, the VH2 comprises the amino acid sequence of SEQ ID NO: 179, and the VL2 comprises the amino acid sequence of SEQ ID NO: 1248. In some such antibodies, the VH2 comprises the amino acid sequence of SEQ ID NO: 2113, and the VL2 comprises the amino acid sequence of SEQ ID NO: 2231. In some such antibodies, the VH2 comprises the amino acid sequence of SEQ ID NO: 1967, and the VL2 comprises the amino acid sequence of SEQ ID NO: 1248. In some such antibodies, the VH2 comprises the amino acid sequence of any one of SEQ ID NOS: 1927-2132, and the VL2 comprises the amino acid sequence of any one of SEQ ID NOS: 2133-2232. In some such antibodies, the VH2 comprises the amino acid sequence of SEQ ID NO: 179, and the VL2 comprises the amino acid sequence of SEQ ID NO: 183. In some such antibodies, the VH2 and the VL2 comprise any pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH2 and the VL2 comprise any pair of anti-IL23 variable heavy domain and variable light domain in Tables 26 and 29-34. In some such antibodies, the VH2 or VL2 can comprise variants of any of the above in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of any one of SEQ ID NOS: 2241, 2253, 2259, 1674, and 1200, (ii) the VL1 comprises a vlCDR1, a vlCDR2, and a vlCDR3 contained within a variable light domain comprising the amino acid sequence of any one of SEQ ID NOS: 2243 and 1204, (iii) the VH2 comprises a vhCDR1, a vhCDR2, and a vhCDR3 contained within a variable heavy domain comprising the amino acid sequence of any one of SEQ ID NOS: 2233, 2235, and 179, and (iv) the VL2 comprises a vlCDR1, a vlCDR2, and a vlCDR3 contained within a variable light domain comprising the amino acid sequence of any one of SEQ ID NOS: 2234, 2237, and 1248, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102 or 1201, a vhCDR2 comprising the amino acid sequence of SEQ ID NO: 1924 or 1202, and a vhCDR3 comprising the amino acid sequence of SEQ ID NO: 1203 or 104, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182 or 2236, and (iv) the VL2 comprises a vlCDR1 comprising the amino acid sequence of SEQ ID NO: 184, a vlCDR2 comprising the amino acid sequence of SEQ ID NO: 1249 or 2238, and a vlCDR3 comprising the amino acid sequence of SEQ ID NO: 1250. In some such antibodies, the VH1 comprises the amino acid sequence of any one of SEQ ID NOS: 2241, 2253, 2259, 1674, and 1200, (ii) the VL1 comprises the amino acid sequence of any one of SEQ ID NOS: 2243 and 1204, (iii) the VH2 comprises the amino acid sequence of any one of SEQ ID NOS: 2233, 2235, and 179, and (iv) the VL2 comprises the amino acid sequence of any one of SEQ ID NOS: 2234, 2237, and 1248, or variants thereof in which an N-terminal glutamine or glutamate is replaced with pyroglutamate. In some such antibodies, the bispecific antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the bispecific antibody specifically binds human TL1A with a K_{D} of less than about 3E-11 M, less than about 2.9E-11 M, less than about 2.8E-11 M, or less than about 2.7E-11 M. In some such antibodies, the bispecific antibody inhibits TL1A activity in a TL1A luciferase reporter assay with an IC₅₀ of less than about 4.5 nM. In some such antibodies, the bispecific antibody specifically binds human IL23 with a K_{D} of less than about 1.1E-10 M as measured by surface plasmon resonance or Biacore at 25° C., optionally wherein the bispecific antibody specifically binds human IL23 with a K_{D} of less than about 6E-11 M, less than about 5.9E-11 M, less than about 5.8E-11 M, or less than about 5.7E-11 M. In some such antibodies, the bispecific antibody inhibits IL23 activity in an IL23 luciferase reporter assay with an IC₅₀ of less than about 65 pM, less than about 64 pM, or less than about 63 pM, optionally wherein the bispecific antibody inhibits TL23 activity with an IC₅₀ of less than about 60 pM, less than about 59 pM, less than about 58 pM, less than about 57 pM, or less than about 56 pM.

In some such antibodies, the VH1 comprises a vhCDR1, a vhCDR2, and a vhCDR3 contained within a variable heavy domain comprising the amino acid sequence of any one of SEQ ID NOS: 2241, 2253, and 2259, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233 or 2235, and (iv) the VL2 comprises a vlCDR1, a vlCDR2, and a vlCDR3 contained within a variable light domain comprising the amino acid sequence of SEQ ID NO: 2234 or 2237, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102 or 1201, a vhCDR2 comprising the amino acid sequence of SEQ ID NO: 1924 or 1202, and a vhCDR3 comprising the amino acid sequence of SEQ ID NO: 1203 or 104, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182 or 2236, and (iv) the VL2 comprises a vlCDR1 comprising the amino acid sequence of SEQ ID NO: 184, a vlCDR2 comprising the amino acid sequence of SEQ ID NO: 1249 or 2238, and a vlCDR3 comprising the amino acid sequence of SEQ ID NO: 1250. In some such antibodies, the VH1 comprises the amino acid sequence of any one of SEQ ID NOS: 2241, 2253, and 2259, (ii) the VL1 comprises the amino acid sequence of SEQ ID NO: 2243, (iii) the VH2 comprises the amino acid sequence of SEQ ID NO: 2233 or 2235, and (iv) the VL2 comprises the amino acid sequence of SEQ ID NO: 2234 or 2237, or variants thereof in which an N-terminal glutamine or glutamate is replaced with pyroglutamate. In some such antibodies, the bispecific antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the bispecific antibody specifically binds human TL1A with a K_{D} of less than about 3E-11 M, less than about 2.9E-11 M, less than about 2.8E-11 M, or less than about 2.7E-11 M. In some such antibodies, the bispecific antibody inhibits TL1A activity in a TL1A luciferase reporter assay with an IC₅₀ of less than about 4.5 nM. In some such antibodies, the bispecific antibody specifically binds human TL23 with a K_{D} of less than about 1.1E-10 M as measured by surface plasmon resonance or Biacore at 25° C., optionally wherein the bispecific antibody specifically binds human IL23 with a K_{D} of less than about 6E-11 M, less than about 5.9E-11 M, less than about 5.8E-11 M, or less than about 5.7E-11 M. In some such antibodies, the bispecific antibody inhibits IL23 activity in an TL23 luciferase reporter assay with an IC₅₀ of less than about 65 pM, less than about 64 pM, or less than about 63 pM, optionally wherein the bispecific antibody inhibits IL23 activity with an IC₅₀ of less than about 60 pM, less than about 59 pM, less than about 58 pM, less than about 57 pM, or less than about 56 pM.

In some such antibodies, the VH1 comprises a vhCDR1, a vhCDR2, and a vhCDR3 contained within a variable heavy domain comprising the amino acid sequence of SEQ ID NO: 2241, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence of SEQ ID NO: 2241, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234, or variants thereof in which an N-terminal glutamine or glutamate is replaced with pyroglutamate. In some such antibodies, the bispecific antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the bispecific antibody specifically binds human TL1A with a K_{D} of less than about 3E-11 M, less than about 2.9E-11 M, less than about 2.8E-11 M, or less than about 2.7E-11 M. In some such antibodies, the bispecific antibody inhibits TL1A activity in a TL1A luciferase reporter assay with an IC₅₀ of less than about 4.5 nM. In some such antibodies, the bispecific antibody specifically binds human IL23 with a K_{D} of less than about 1.1E-10 M as measured by surface plasmon resonance or Biacore at 25° C., optionally wherein the bispecific antibody specifically binds human IL23 with a K_{D} of less than about 6E-11 M, less than about 5.9E-11 M, less than about 5.8E-11 M, or less than about 5.7E-11 M. In some such antibodies, the bispecific antibody inhibits IL23 activity in an IL23 luciferase reporter assay with an IC₅₀ of less than about 65 pM, less than about 64 pM, or less than about 63 pM, optionally wherein the bispecific antibody inhibits IL23 activity with an IC₅₀ of less than about 60 pM, less than about 59 pM, less than about 58 pM, less than about 57 pM, or less than about 56 pM.

In some such antibodies, the VH1 comprises a vhCDR1, a vhCDR2, and a vhCDR3 contained within a variable heavy domain comprising the amino acid sequence of SEQ ID NO: 2241, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2235, and (iv) the VL2 comprises a vlCDR1, a vlCDR2, and a vlCDR3 contained within a variable light domain comprising the amino acid sequence of SEQ ID NO: 2237, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 2236, and (iv) the VL2 comprises a vlCDR1 comprising the amino acid sequence of SEQ ID NO: 184, a vlCDR2 comprising the amino acid sequence of SEQ ID NO: 2238, and a vlCDR3 comprising the amino acid sequence of SEQ ID NO: 1250. In some such antibodies, the VH1 comprises the amino acid sequence of SEQ ID NO: 2241, (ii) the VL1 comprises the amino acid sequence of SEQ ID NO: 2243, (iii) the VH2 comprises the amino acid sequence of SEQ ID NO: 2235, and (iv) the VL2 comprises the amino acid sequence of SEQ ID NO: 2237, or variants thereof in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of SEQ ID NO: 2253, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence of SEQ ID NO: 2253, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234, or variants thereof in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of SEQ ID NO: 2259, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 104, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence of SEQ ID NO: 2259, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234, or variants thereof in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of SEQ ID NO: 1674, (ii) the VL1 comprises a vlCDR1, a vlCDR2, and a vlCDR3 contained within a variable light domain comprising the amino acid sequence of SEQ ID NO: 1204, (iii) the VH2 comprises a vhCDR1, a vhCDR2, and a vhCDR3 contained within a variable heavy domain comprising the amino acid sequence of SEQ ID NO: 179, and (iv) the VL2 comprises a vlCDR1, a vlCDR2, and a vlCDR3 contained within a variable light domain comprising the amino acid sequence of SEQ ID NO: 1248, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence of SEQ ID NO: 1674, (ii) the VL1 comprises the amino acid sequence of SEQ ID NO: 1204, (iii) the VH2 comprises the amino acid sequence of SEQ ID NO: 179, and (iv) the VL2 comprises the amino acid sequence of SEQ ID NO: 1248, or variants thereof in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of SEQ ID NO: 1200, (ii) the VL1 comprises a vlCDR1, a vlCDR2, and a vlCDR3 contained within a variable light domain comprising the amino acid sequence of SEQ ID NO: 1204, (iii) the VH2 comprises a vhCDR1, a vhCDR2, and a vhCDR3 contained within a variable heavy domain comprising the amino acid sequence of SEQ ID NO: 179, and (iv) the VL2 comprises a vlCDR1, a vlCDR2, and a vlCDR3 contained within a variable light domain comprising the amino acid sequence of SEQ ID NO: 1248, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence SEQ ID NO: 1200, (ii) the VL1 comprises the amino acid sequence of SEQ ID NO: 1204, (iii) the VH2 comprises the amino acid sequence of SEQ ID NO: 179, and (iv) the VL2 comprises the amino acid sequence of SEQ ID NO: 1248, or variants thereof in which an N-terminal glutamine or glutamate is replaced with pyroglutamate.

In some such antibodies, the first Fc domain and the second Fc domain comprise a set of heterodimerization skew variants. In some such antibodies, the first Fc domain and the second Fc domain each comprise one or more ablation variants. In some such antibodies, the first monomer or the second monomer comprises one or more pI variants. In some such antibodies, the first heavy chain constant domain comprises amino acid substitutions E233P/L234V/L235A/G236del/S267K and/or the second heavy chain constant domain comprises amino acid substitutions E233P/L234V/L235A/G236del/S267K, wherein numbering is according to EU numbering. In some such antibodies, the first heavy chain constant domain comprises amino acid substitutions M428L/N434S and/or the second heavy chain constant domain comprises amino acid substitutions M428L/N434S, wherein numbering is according to EU numbering. In some such antibodies, the first heavy chain constant domain comprises amino acid substitutions E233P/L234V/L235A/G236del/S267K and M428L/N434S and/or the second heavy chain constant domain comprises amino acid substitutions E233P/L234V/L235A/G236del/S267K and M428L/N434S, wherein numbering is according to EU numbering. In some such antibodies, the first heavy chain constant domain or the second heavy chain constant domain comprises amino acid substitutions L368D/K370S, wherein numbering is according to EU numbering. In some such antibodies, the first heavy chain constant domain or the second heavy chain constant domain comprises amino acid substitutions N208D/Q295E/N384D/Q418E/N421D, wherein numbering is according to EU numbering. In some such antibodies, the first heavy chain constant domain or the second heavy chain constant domain comprises amino acid substitutions E233P/L234V/L235A/G236del/S267K, M428L/N434S, L368D/K370S, and N208D/Q295E/N384D/Q418E/N421D, wherein numbering is according to EU numbering. In some such antibodies, the first heavy chain constant domain or the second heavy chain constant domain comprises amino acid substitutions E357Q/S364K, wherein numbering is according to EU numbering. In some such antibodies, the first heavy chain constant domain comprises amino acid substitutions L368D/K370S, and the second heavy chain constant domain comprises amino acid substitutions E357Q/S364K, wherein numbering is according to EU numbering. In some such antibodies, the first heavy chain constant domain or the second heavy chain constant domain comprises amino acid substitutions E233P/L234V/L235A/G236del/S267K, M428L/N434S, and E357Q/S364K, wherein numbering is according to EU numbering. In some such antibodies, the first heavy chain constant domain comprises amino acid substitutions E233P/L234V/L235A/G236del/S267K, M428L/N434S, L368D/K370S, and N208D/Q295E/N384D/Q418E/N421D, wherein numbering is according to EU numbering, and the second heavy chain constant domain comprises amino acid substitutions E233P/L234V/L235A/G236del/S267K, M428L/N434S, and E357Q/S364K, wherein numbering is according to EU numbering.

In some such antibodies, the first heavy chain constant domain is of isotype IgG1, and the second heavy chain constant domain is of isotype IgG1. In some such antibodies, the bispecific antibody is a chimeric or humanized bispecific antibody.

In some such antibodies, the first heavy chain comprises the amino acid sequence of SEQ ID NO: 2240 or a variant thereof in which the N-terminal glutamine is replaced with pyroglutamate and/or the C-terminal lysine or the C-terminal lysine and glycine are removed, and the first light chain comprises the amino acid sequence of SEQ ID NO: 2242. In some such antibodies, the first heavy chain comprises the amino acid sequence of SEQ ID NO: 2252 or a variant thereof in which the N-terminal glutamine is replaced with pyroglutamate and/or the C-terminal lysine or the C-terminal lysine and glycine are removed, and the first light chain comprises the amino acid sequence of SEQ ID NO: 2242. In some such antibodies, the first heavy chain comprises the amino acid sequence of SEQ ID NO: 2258 or a variant thereof in which the N-terminal glutamine is replaced with pyroglutamate and/or the C-terminal lysine or the C-terminal lysine and glycine are removed, and the first light chain comprises the amino acid sequence of SEQ ID NO: 2242. In some such antibodies, the first heavy chain is at least 90%, at least 95%, at least 96%, at least 97%, at least 98%, or at least 99% identical to the amino acid sequence of SEQ ID NO: 2240, and the first light chain is at least 90%, at least 95%, at least 96%, at least 97%, at least 98%, or at least 99% identical to the amino acid sequence of SEQ ID NO: 2242. In some such antibodies, the first heavy chain is at least 90%, at least 95%, at least 96%, at least 97%, at least 98%, or at least 99% identical to the amino acid sequence of SEQ ID NO: 2252, and the first light chain is at least 90%, at least 95%, at least 96%, at least 97%, at least 98%, or at least 99% identical to the amino acid sequence of SEQ ID NO: 2242. In some such antibodies, the first heavy chain is at least 90%, at least 95%, at least 96%, at least 97%, at least 98%, or at least 99% identical to the amino acid sequence of SEQ ID NO: 2258, and the first light chain is at least 90%, at least 95%, at least 96%, at least 97%, at least 98%, or at least 99% identical to the amino acid sequence of SEQ ID NO: 2242.

In some such antibodies, the second heavy chain comprises the amino acid sequence of SEQ ID NO: 2244 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2245. In some such antibodies, the second heavy chain comprises the amino acid sequence of SEQ ID NO: 2250 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2251. In some such antibodies, the second heavy chain is at least 90%, at least 95%, at least 96%, at least 97%, at least 98%, or at least 99% identical to the amino acid sequence of SEQ ID NO: 2244, and the second light chain is at least 90%, at least 95%, at least 96%, at least 97%, at least 98%, or at least 99% identical to the amino acid sequence of SEQ ID NO: 2245. In some such antibodies, the second heavy chain is at least 90%, at least 95%, at least 96%, at least 97%, at least 98%, or at least 99% identical to the amino acid sequence of SEQ ID NO: 2250, and the second light chain is at least 90%, at least 95%, at least 96%, at least 97%, at least 98%, or at least 99% identical to the amino acid sequence of SEQ ID NO: 2251.

In some such antibodies, the first heavy chain comprises the amino acid sequence of SEQ ID NO: 1132 or a variant thereof in which the N-terminal glutamine is replaced with pyroglutamate and/or the C-terminal lysine or the C-terminal lysine and glycine are removed, the first light chain comprises the amino acid sequence of SEQ ID NO: 1134, the second heavy chain comprises the amino acid sequence of SEQ ID NO: 1133 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 1135. In some such antibodies, the first heavy chain comprises the amino acid sequence of SEQ ID NO: 1136 or a variant thereof in which the N-terminal glutamine is replaced with pyroglutamate and/or the C-terminal lysine or the C-terminal lysine and glycine are removed, the first light chain comprises the amino acid sequence of SEQ ID NO: 1138, the second heavy chain comprises the amino acid sequence of SEQ ID NO: 1137 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 1139. In some such antibodies, the first heavy chain comprises the amino acid sequence of SEQ ID NO: 1084 or a variant thereof in which the N-terminal glutamine is replaced with pyroglutamate and/or the C-terminal lysine or the C-terminal lysine and glycine are removed, the first light chain comprises the amino acid sequence of SEQ ID NO: 1086, the second heavy chain comprises the amino acid sequence of SEQ ID NO: 1085 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 1087. In some such antibodies, the first heavy chain comprises the amino acid sequence of SEQ ID NO: 1108 or a variant thereof in which the N-terminal glutamine is replaced with pyroglutamate and/or the C-terminal lysine or the C-terminal lysine and glycine are removed, the first light chain comprises the amino acid sequence of SEQ ID NO: 1110, the second heavy chain comprises the amino acid sequence of SEQ ID NO: 1109 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 1111. In some such antibodies, the first heavy chain comprises the amino acid sequence of SEQ ID NO: 1088 or a variant thereof in which the N-terminal glutamine is replaced with pyroglutamate and/or the C-terminal lysine or the C-terminal lysine and glycine are removed, the first light chain comprises the amino acid sequence of SEQ ID NO: 1090, the second heavy chain comprises the amino acid sequence of SEQ ID NO: 1089 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 1091. In some such antibodies, the first heavy chain comprises the amino acid sequence of SEQ ID NO: 1092 or a variant thereof in which the N-terminal glutamine is replaced with pyroglutamate and/or the C-terminal lysine or the C-terminal lysine and glycine are removed, the first light chain comprises the amino acid sequence of SEQ ID NO: 1094, the second heavy chain comprises the amino acid sequence of SEQ ID NO: 1093 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 1095. In some such antibodies, the first heavy chain comprises the amino acid sequence of SEQ ID NO: 1112 or a variant thereof in which the N-terminal glutamine is replaced with pyroglutamate and/or the C-terminal lysine or the C-terminal lysine and glycine are removed, the first light chain comprises the amino acid sequence of SEQ ID NO: 1114, the second heavy chain comprises the amino acid sequence of SEQ ID NO: 1113 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 1115. In some such antibodies, the first heavy chain comprises the amino acid sequence of SEQ ID NO: 1116 or a variant thereof in which the N-terminal glutamine is replaced with pyroglutamate and/or the C-terminal lysine or the C-terminal lysine and glycine are removed, the first light chain comprises the amino acid sequence of SEQ ID NO: 1118, the second heavy chain comprises the amino acid sequence of SEQ ID NO: 1117 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 1119. In some such antibodies, the first heavy chain comprises the amino acid sequence of SEQ ID NO: 1120 or a variant thereof in which the N-terminal glutamine is replaced with pyroglutamate and/or the C-terminal lysine or the C-terminal lysine and glycine are removed, the first light chain comprises the amino acid sequence of SEQ ID NO: 1122, the second heavy chain comprises the amino acid sequence of SEQ ID NO: 1121 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 1123. In some such antibodies, the first heavy chain comprises the amino acid sequence of SEQ ID NO: 1124 or a variant thereof in which the N-terminal glutamine is replaced with pyroglutamate and/or the C-terminal lysine or the C-terminal lysine and glycine are removed, the first light chain comprises the amino acid sequence of SEQ ID NO: 1126, the second heavy chain comprises the amino acid sequence of SEQ ID NO: 1125 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 1127. In some such antibodies, the first heavy chain comprises the amino acid sequence of SEQ ID NO: 1128 or a variant thereof in which the N-terminal glutamine is replaced with pyroglutamate and/or the C-terminal lysine or the C-terminal lysine and glycine are removed, the first light chain comprises the amino acid sequence of SEQ ID NO: 1130, the second heavy chain comprises the amino acid sequence of SEQ ID NO: 1129 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 1131. In some such antibodies, the first heavy chain comprises the amino acid sequence of SEQ ID NO: 1140, the first light chain comprises the amino acid sequence of SEQ ID NO: 1142, the second heavy chain comprises the amino acid sequence of SEQ ID NO: 1141, and the second light chain comprises the amino acid sequence of SEQ ID NO: 1143. In some such antibodies, the first heavy chain is at least 90%, at least 95%, at least 96%, at least 97%, at least 98%, or at least 99% identical to the amino acid sequence of SEQ ID NO: 1132, the first light chain is at least 90%, at least 95%, at least 96%, at least 97%, at least 98%, or at least 99% identical to the amino acid sequence of SEQ ID NO:1134, the second heavy chain is at least 90%, at least 95%, at least 96%, at least 97%, at least 98%, or at least 99% identical to the amino acid sequence of SEQ ID NO: 1133, and the second light chain is at least 90%, at least 95%, at least 96%, at least 97%, at least 98%, or at least 99% identical to the amino acid sequence of SEQ ID NO: 1135. In some such antibodies, the first heavy chain is at least 90%, at least 95%, at least 96%, at least 97%, at least 98%, or at least 99% identical to the amino acid sequence of SEQ ID NO: 1136, the first light chain is at least 90%, at least 95%, at least 96%, at least 97%, at least 98%, or at least 99% identical to the amino acid sequence of SEQ ID NO: 1138, the second heavy chain is at least 90%, at least 95%, at least 96%, at least 97%, at least 98%, or at least 99% identical to the amino acid sequence of SEQ ID NO: 1137, and the second light chain is at least 90%, at least 95%, at least 96%, at least 97%, at least 98%, or at least 99% identical to the amino acid sequence of SEQ ID NO: 1139. In some such antibodies, the first heavy chain is at least 90%, at least 95%, at least 96%, at least 97%, at least 98%, or at least 99% identical to the amino acid sequence of SEQ ID NO: 1084, the first light chain is at least 90%, at least 95%, at least 96%, at least 97%, at least 98%, or at least 99% identical to the amino acid sequence of SEQ ID NO: 1086, the second heavy chain is at least 90%, at least 95%, at least 96%, at least 97%, at least 98%, or at least 99% identical to the amino acid sequence of SEQ ID NO: 1085, and the second light chain is at least 90%, at least 95%, at least 96%, at least 97%, at least 98%, or at least 99% identical to the amino acid sequence of SEQ ID NO: 1087. In some such antibodies, the first heavy chain is at least 90%, at least 95%, at least 96%, at least 97%, at least 98%, or at least 99% identical to the amino acid sequence of SEQ ID NO: 1108, the first light chain is at least 90%, at least 95%, at least 96%, at least 97%, at least 98%, or at least 99% identical to the amino acid sequence of SEQ ID NO: 1110, the second heavy chain is at least 90%, at least 95%, at least 96%, at least 97%, at least 98%, or at least 99% identical to the amino acid sequence of SEQ ID NO: 1109, and the second light chain is at least 90%, at least 95%, at least 96%, at least 97%, at least 98%, or at least 99% identical to the amino acid sequence of SEQ ID NO: 1111. In some such antibodies, the first heavy chain is at least 90%, at least 95%, at least 96%, at least 97%, at least 98%, or at least 99% identical to the amino acid sequence of SEQ ID NO: 1088, the first light chain is at least 90%, at least 95%, at least 96%, at least 97%, at least 98%, or at least 99% identical to the amino acid sequence of SEQ ID NO: 1090, the second heavy chain is at least 90%, at least 95%, at least 96%, at least 97%, at least 98%, or at least 99% identical to the amino acid sequence of SEQ ID NO: 1089, and the second light chain is at least 90%, at least 95%, at least 96%, at least 97%, at least 98%, or at least 99% identical to the amino acid sequence of SEQ ID NO: 1091. In some such antibodies, the first heavy chain is at least 90%, at least 95%, at least 96%, at least 97%, at least 98%, or at least 99% identical to the amino acid sequence of SEQ ID NO: 1092, the first light chain is at least 90%, at least 95%, at least 96%, at least 97%, at least 98%, or at least 99% identical to the amino acid sequence of SEQ ID NO: 1094, the second heavy chain is at least 90%, at least 95%, at least 96%, at least 97%, at least 98%, or at least 99% identical to the amino acid sequence of SEQ ID NO: 1093, and the second light chain is at least 90%, at least 95%, at least 96%, at least 97%, at least 98%, or at least 99% identical to the amino acid sequence of SEQ ID NO: 1095. In some such antibodies, the first heavy chain is at least 90%, at least 95%, at least 96%, at least 97%, at least 98%, or at least 99% identical to the amino acid sequence of SEQ ID NO: 1112, the first light chain is at least 90%, at least 95%, at least 96%, at least 97%, at least 98%, or at least 99% identical to the amino acid sequence of SEQ ID NO: 1114, the second heavy chain is at least 90%, at least 95%, at least 96%, at least 97%, at least 98%, or at least 99% identical to the amino acid sequence of SEQ ID NO: 1113, and the second light chain is at least 90%, at least 95%, at least 96%, at least 97%, at least 98%, or at least 99% identical to the amino acid sequence of SEQ ID NO: 1115. In some such antibodies, the first heavy chain is at least 90%, at least 95%, at least 96%, at least 97%, at least 98%, or at least 99% identical to the amino acid sequence of SEQ ID NO: 1116, the first light chain is at least 90%, at least 95%, at least 96%, at least 97%, at least 98%, or at least 99% identical to the amino acid sequence of SEQ ID NO: 1118, the second heavy chain is at least 90%, at least 95%, at least 96%, at least 97%, at least 98%, or at least 99% identical to the amino acid sequence of SEQ ID NO: 1117, and the second light chain is at least 90%, at least 95%, at least 96%, at least 97%, at least 98%, or at least 99% identical to the amino acid sequence of SEQ ID NO: 1119. In some such antibodies, the first heavy chain is at least 90%, at least 95%, at least 96%, at least 97%, at least 98%, or at least 99% identical to the amino acid sequence of SEQ ID NO: 1120, the first light chain is at least 90%, at least 95%, at least 96%, at least 97%, at least 98%, or at least 99% identical to the amino acid sequence of SEQ ID NO: 1122, the second heavy chain is at least 90%, at least 95%, at least 96%, at least 97%, at least 98%, or at least 99% identical to the amino acid sequence of SEQ ID NO: 1121, and the second light chain is at least 90%, at least 95%, at least 96%, at least 97%, at least 98%, or at least 99% identical to the amino acid sequence of SEQ ID NO: 1123. In some such antibodies, the first heavy chain is at least 90%, at least 95%, at least 96%, at least 97%, at least 98%, or at least 99% identical to the amino acid sequence of SEQ ID NO: 1124, the first light chain is at least 90%, at least 95%, at least 96%, at least 97%, at least 98%, or at least 99% identical to the amino acid sequence of SEQ ID NO: 1126, the second heavy chain is at least 90%, at least 95%, at least 96%, at least 97%, at least 98%, or at least 99% identical to the amino acid sequence of SEQ ID NO: 1125, and the second light chain is at least 90%, at least 95%, at least 96%, at least 97%, at least 98%, or at least 99% identical to the amino acid sequence of SEQ ID NO: 1127. In some such antibodies, the first heavy chain is at least 90%, at least 95%, at least 96%, at least 97%, at least 98%, or at least 99% identical to the amino acid sequence of SEQ ID NO: 1128, the first light chain is at least 90%, at least 95%, at least 96%, at least 97%, at least 98%, or at least 99% identical to the amino acid sequence of SEQ ID NO: 1130, the second heavy chain is at least 90%, at least 95%, at least 96%, at least 97%, at least 98%, or at least 99% identical to the amino acid sequence of SEQ ID NO: 1129, and the second light chain is at least 90%, at least 95%, at least 96%, at least 97%, at least 98%, or at least 99% identical to the amino acid sequence of SEQ ID NO: 1131. In some such antibodies, the first heavy chain is at least 90%, at least 95%, at least 96%, at least 97%, at least 98%, or at least 99% identical to the amino acid sequence of SEQ ID NO: 1140, the first light chain is at least 90%, at least 95%, at least 96%, at least 97%, at least 98%, or at least 99% identical to the amino acid sequence of SEQ ID NO: 1142, the second heavy chain is at least 90%, at least 95%, at least 96%, at least 97%, at least 98%, or at least 99% identical to the amino acid sequence of SEQ ID NO: 1141, and the second light chain is at least 90%, at least 95%, at least 96%, at least 97%, at least 98%, or at least 99% identical to the amino acid sequence of SEQ ID NO: 1143. In some such antibodies, the first heavy chain, the first light chain, the second heavy chain, and the second light chain comprise any set of four chains from XENP53412, XENP53413, XENP53369, XENP53375, XENP53370, XENP53371, XENP53376, XENP53377, XENP53378, XENP53379, XENP53380, or XENP53414. In some such antibodies, the first heavy chain, the first light chain, the second heavy chain, and the second light chain comprise any set of four chains from Tables 29 and 30.

In some such antibodies, the first heavy chain comprises the amino acid sequence of SEQ ID NO: 2240 or a variant thereof in which the N-terminal glutamine is replaced with pyroglutamate and/or the C-terminal lysine or the C-terminal lysine and glycine are removed, the first light chain comprises the amino acid sequence of SEQ ID NO: 2242, the second heavy chain comprises the amino acid sequence of SEQ ID NO: 2244 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2245. In some such antibodies, the first heavy chain comprises the amino acid sequence of SEQ ID NO: 2240 or a variant thereof in which the N-terminal glutamine is replaced with pyroglutamate and/or the C-terminal lysine or the C-terminal lysine and glycine are removed, the first light chain comprises the amino acid sequence of SEQ ID NO: 2242, the second heavy chain comprises the amino acid sequence of SEQ ID NO: 2250 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2251. In some such antibodies, the first heavy chain comprises the amino acid sequence of SEQ ID NO: 2252 or a variant thereof in which the N-terminal glutamine is replaced with pyroglutamate and/or the C-terminal lysine or the C-terminal lysine and glycine are removed, the first light chain comprises the amino acid sequence of SEQ ID NO: 2242, the second heavy chain comprises the amino acid sequence of SEQ ID NO: 2244 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2245. In some such antibodies, the first heavy chain comprises the amino acid sequence of SEQ ID NO: 2258 or a variant thereof in which the N-terminal glutamine is replaced with pyroglutamate and/or the C-terminal lysine or the C-terminal lysine and glycine are removed, the first light chain comprises the amino acid sequence of SEQ ID NO: 2242, the second heavy chain comprises the amino acid sequence of SEQ ID NO: 2244 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2245. In some such antibodies, the first heavy chain is at least 90%, at least 95%, at least 96%, at least 97%, at least 98%, or at least 99% identical to the amino acid sequence of SEQ ID NO: 2240, the first light chain is at least 90%, at least 95%, at least 96%, at least 97%, at least 98%, or at least 99% identical to the amino acid sequence of SEQ ID NO: 2242, the second heavy chain is at least 90%, at least 95%, at least 96%, at least 97%, at least 98%, or at least 99% identical to the amino acid sequence of SEQ ID NO: 2244, and the second light chain is at least 90%, at least 95%, at least 96%, at least 97%, at least 98%, or at least 99% identical to the amino acid sequence of SEQ ID NO: 2245. In some such antibodies, the first heavy chain is at least 90%, at least 95%, at least 96%, at least 97%, at least 98%, or at least 99% identical to the amino acid sequence of SEQ ID NO: 2240, the first light chain is at least 90%, at least 95%, at least 96%, at least 97%, at least 98%, or at least 99% identical to the amino acid sequence of SEQ ID NO: 2242, the second heavy chain is at least 90%, at least 95%, at least 96%, at least 97%, at least 98%, or at least 99% identical to the amino acid sequence of SEQ ID NO: 2250, and the second light chain is at least 90%, at least 95%, at least 96%, at least 97%, at least 98%, or at least 99% identical to the amino acid sequence of SEQ ID NO: 2251. In some such antibodies, the first heavy chain is at least 90%, at least 95%, at least 96%, at least 97%, at least 98%, or at least 99% identical to the amino acid sequence of SEQ ID NO: 2252, the first light chain is at least 90%, at least 95%, at least 96%, at least 97%, at least 98%, or at least 99% identical to the amino acid sequence of SEQ ID NO: 2242, the second heavy chain is at least 90%, at least 95%, at least 96%, at least 97%, at least 98%, or at least 99% identical to the amino acid sequence of SEQ ID NO: 2244, and the second light chain is at least 90%, at least 95%, at least 96%, at least 97%, at least 98%, or at least 99% identical to the amino acid sequence of SEQ ID NO: 2245. In some such antibodies, the first heavy chain is at least 90%, at least 95%, at least 96%, at least 97%, at least 98%, or at least 99% identical to the amino acid sequence of SEQ ID NO: 2258, the first light chain is at least 90%, at least 95%, at least 96%, at least 97%, at least 98%, or at least 99% identical to the amino acid sequence of SEQ ID NO: 2242, the second heavy chain is at least 90%, at least 95%, at least 96%, at least 97%, at least 98%, or at least 99% identical to the amino acid sequence of SEQ ID NO: 2244, and the second light chain is at least 90%, at least 95%, at least 96%, at least 97%, at least 98%, or at least 99% identical to the amino acid sequence of SEQ ID NO: 2245.

In some such antibodies, the bispecific antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the bispecific antibody specifically binds human TL1A with a K_{D} of less than about 3E-11 M, less than about 2.9E-11 M, less than about 2.8E-11 M, or less than about 2.7E-11 M. In some such antibodies, the bispecific antibody inhibits TL1A activity in a TL1A luciferase reporter assay with an IC₅₀ of less than about 4.5 nM. In some such antibodies, the bispecific antibody specifically binds human IL23 with a K_{D} of less than about 1.1E-10 M as measured by surface plasmon resonance or Biacore at 25° C., optionally wherein the bispecific antibody specifically binds human IL23 with a K_{D} of less than about 6E-11 M, less than about 5.9E-11 M, less than about 5.8E-11 M, or less than about 5.7E-11 M. In some such antibodies, the bispecific antibody inhibits IL23 activity in an IL23 luciferase reporter assay with an IC₅₀ of less than about 65 pM, less than about 64 pM, or less than about 63 pM, optionally wherein the bispecific antibody inhibits IL23 activity with an IC₅₀ of less than about 60 pM, less than about 59 pM, less than about 58 pM, less than about 57 pM, or less than about 56 pM.

In another aspect, provided are bispecific or heterodimeric antibodies comprising: (a) a first monomer comprising a first heavy chain comprising a VL1-CH1-hinge-CH2-CH3, wherein VL1 is a first variable light domain, CH1-hinge-CH2-CH3 is a first heavy chain constant domain, and CH2-CH3 is a first Fc domain; (b) a second monomer comprising a second heavy chain comprising a VH2-CH1-hinge-CH2-CH3, wherein VH2 is a second variable heavy domain, CH1-hinge-CH2-CH3 is a second heavy chain constant domain, and CH2-CH3 is a second Fc domain; (c) a first light chain comprising a VH1-CL, wherein VH1 is a first variable heavy domain, and CL is a first light chain constant domain; and (d) a second light chain comprising a VL2-CL, wherein VL2 is a second variable light domain, and CL is a second light chain constant domain, wherein the VH1 and VL1 together form a first antigen-binding domain (ABD), and the VH2 and the VL2 together form a second ABD, wherein the first ABD is a TL1A-binding domain and the second ABD is an IL23-binding domain, or wherein the first ABD is an IL23-binding domain and the second ABD is a TL1A-binding domain. For example, any of the above antibodies can be in this format.

In some such antibodies, the second heavy chain constant domain and the second light chain constant domain comprise a set of electrostatic steering variants at positions 213 and 218 of the second heavy chain constant domain according to EU numbering and positions 122 and 123 of the second light chain constant domain according to Kabat numbering. In some such antibodies, the second heavy chain constant domain and the second light chain constant domain comprise a set of electrostatic steering variants as depicted in FIG. 4 or Table 27. In some such antibodies, the second heavy chain constant domain comprises amino acid substitutions K213E/K218D according to EU numbering, and the second light chain constant domain comprises amino acid substitutions D122K/E123K according to Kabat numbering.

In some such antibodies, the first ABD is a TL1A-binding domain and the second ABD is an IL23-binding domain.

In some such antibodies, the VH1 and VL1 comprise a variable heavy domain comprising a vhCDR1, a vhCDR2, and a vhCDR3 contained within a variable heavy domain comprising the amino acid sequence of any one of SEQ ID NOS: 1674, 1200, 2241, 2253, and 2259, and a variable light domain comprising a vlCDR1, a vlCDR2, and a vlCDR3 contained within a variable light domain comprising the amino acid sequence of any one of SEQ ID NOS: 1204 and 2243. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 1674, and a variable light domain comprising a vlCDR1, a vlCDR2, and a vlCDR3 contained within a variable light domain comprising the amino acid sequence of SEQ ID NO: 1204. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 1200, and a variable light domain comprising a vlCDR1, a vlCDR2, and a vlCDR3 contained within a variable light domain comprising the amino acid sequence of SEQ ID NO: 1204. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 2241, and a variable light domain comprising a vlCDR1, a vlCDR2, and a vlCDR3 contained within a variable light domain comprising the amino acid sequence of SEQ ID NO: 2243. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 2253, and a variable light domain comprising a vlCDR1, a vlCDR2, and a vlCDR3 contained within a variable light domain comprising the amino acid sequence of SEQ ID NO: 2243. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 2259, and a variable light domain comprising a vlCDR1, a vlCDR2, and a vlCDR3 contained within a variable light domain comprising the amino acid sequence of SEQ ID NO: 2243. In some such antibodies, the VH1 and VL1 comprise a variable heavy domain comprising a vhCDR1, a vhCDR2, and a vhCDR3 contained within a variable heavy domain comprising the amino acid sequence of any one of SEQ ID NOS: 1427-1823 and 2316-2330, and a variable light domain comprising a vlCDR1, a vlCDR2, and a vlCDR3 contained within a variable light domain comprising the amino acid sequence of any one of SEQ ID NOS: 1824-1923. In some such antibodies, the VH1 and VL1 comprise a variable heavy domain comprising a vhCDR1, a vhCDR2, and a vhCDR3 contained within a variable heavy domain comprising the amino acid sequence of any one of SEQ ID NOS: 1674, 1200, 1661, and 2323, and a variable light domain comprising a vlCDR1, a vlCDR2, and a vlCDR3 contained within a variable light domain comprising the amino acid sequence of SEQ ID NO: 1204. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 1661, and a variable light domain comprising a vlCDR1, a vlCDR2, and a vlCDR3 contained within a variable light domain comprising the amino acid sequence of SEQ ID NO: 1204. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 2323, and a variable light domain comprising a vlCDR1, a vlCDR2, and a vlCDR3 contained within a variable light domain comprising the amino acid sequence of SEQ ID NO: 1204. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 101, and a variable light domain comprising a vlCDR1, a vlCDR2, and a vlCDR3 contained within a variable light domain comprising the amino acid sequence of SEQ ID NO: 105. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 109, and a variable light domain comprising a vlCDR1, a vlCDR2, and a vlCDR3 contained within a variable light domain comprising the amino acid sequence of SEQ ID NO: 113. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 117, and a variable light domain comprising a vlCDR1, a vlCDR2, and a vlCDR3 contained within a variable light domain comprising the amino acid sequence of SEQ ID NO: 121. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 125, and a variable light domain comprising a vlCDR1, a vlCDR2, and a vlCDR3 contained within a variable light domain comprising the amino acid sequence of SEQ ID NO: 129. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 133, and a variable light domain comprising a vlCDR1, a vlCDR2, and a vlCDR3 contained within a variable light domain comprising the amino acid sequence of SEQ ID NO: 137. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 141, and a variable light domain comprising a vlCDR1, a vlCDR2, and a vlCDR3 contained within a variable light domain comprising the amino acid sequence of SEQ ID NO: 145. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 149, and a variable light domain comprising a vlCDR1, a vlCDR2, and a vlCDR3 contained within a variable light domain comprising the amino acid sequence of SEQ ID NO: 153. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 157, and a variable light domain comprising a vlCDR1, a vlCDR2, and a vlCDR3 contained within a variable light domain comprising the amino acid sequence of SEQ ID NO: 159. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 161, and a variable light domain comprising a vlCDR1, a vlCDR2, and a vlCDR3 contained within a variable light domain comprising the amino acid sequence of SEQ ID NO: 165. In some such antibodies, the VH1 and VL1 comprise a variable heavy domain comprising a vhCDR1, a vhCDR2, and a vhCDR3 contained within a variable heavy domain comprising the amino acid sequence of SEQ ID NO: 169, and a variable light domain comprising a vlCDR1, a vlCDR2, and a vlCDR3 contained within a variable light domain comprising the amino acid sequence of SEQ ID NO: 173. In some such antibodies, the VH1 and VL1 comprise a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH1 and VL1 comprise a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in Tables 26 and 29-34. Optionally, the CDRs in any of the above antibodies are as defined by the Kabat, Chothia, Kabat+Chothia, AbM, contact, or IMGT definition.

In some such antibodies, the VH1 and VL1 comprise a variable heavy domain comprising a vhCDR1 comprising the amino acid sequence of SEQ ID NO: 102 or 1201, a vhCDR2 comprising the amino acid sequence of SEQ ID NO: 1924 or 1202, and a vhCDR3 comprising the amino acid sequence of SEQ ID NO: 1203 or 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and VL1 comprise a variable heavy domain comprising a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and VL1 comprise a variable heavy domain comprising a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and VL1 comprise a variable heavy domain comprising a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and VL1 comprise a variable heavy domain comprising a vhCDR1 comprising the amino acid sequence of SEQ ID NO: 102, a vhCDR2 comprising the amino acid sequence of SEQ ID NO: 1926, and a vhCDR3 comprising the amino acid sequence of SEQ ID NO: 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and VL1 comprise a variable heavy domain comprising a vhCDR1 comprising the amino acid sequence of SEQ ID NO: 102, a vhCDR2 comprising the amino acid sequence of SEQ ID NO: 103, and a vhCDR3 comprising the amino acid sequence of SEQ ID NO: 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the VH1 and VL1 comprise a variable heavy domain comprising a vhCDR1 comprising the amino acid sequence of SEQ ID NO: 110, a vhCDR2 comprising the amino acid sequence of SEQ ID NO: 111, and a vhCDR3 comprising the amino acid sequence of SEQ ID NO: 112, and a variable light domain comprising a vlCDR1 comprising the amino acid sequence of SEQ ID NO: 114, a vlCDR2 comprising the amino acid sequence of SEQ ID NO: 115, and a vlCDR3 comprising the amino acid sequence of SEQ ID NO: 116. In some such antibodies, the VH1 and VL1 comprise a variable heavy domain comprising a vhCDR1 comprising the amino acid sequence of SEQ ID NO: 118, a vhCDR2 comprising the amino acid sequence of SEQ ID NO: 119, and a vhCDR3 comprising the amino acid sequence of SEQ ID NO: 120, and a variable light domain comprising a vlCDR1 comprising the amino acid sequence of SEQ ID NO: 122, a vlCDR2 comprising the amino acid sequence of SEQ ID NO: 123, and a vlCDR3 comprising the amino acid sequence of SEQ ID NO: 124. In some such antibodies, the VH1 and VL1 comprise a variable heavy domain comprising a vhCDR1 comprising the amino acid sequence of SEQ ID NO: 126, a vhCDR2 comprising the amino acid sequence of SEQ ID NO: 127, and a vhCDR3 comprising the amino acid sequence of SEQ ID NO: 128, and a variable light domain comprising a vlCDR1 comprising the amino acid sequence of SEQ ID NO: 130, a vlCDR2 comprising the amino acid sequence of SEQ ID NO: 131, and a vlCDR3 comprising the amino acid sequence of the amino acid sequence of SEQ ID NO: 132. In some such antibodies, the VH1 and VL1 comprise a variable heavy domain comprising a vhCDR1 comprising the amino acid sequence of SEQ ID NO: 134, a vhCDR2 comprising the amino acid sequence of SEQ ID NO: 135, and a vhCDR3 comprising the amino acid sequence of SEQ ID NO: 136, and a variable light domain comprising a vlCDR1 comprising the amino acid sequence of SEQ ID NO: 138, a vlCDR2 comprising the amino acid sequence of SEQ ID NO: 139, and a vlCDR3 comprising the amino acid sequence of the amino acid sequence of SEQ ID NO: 140. In some such antibodies, the VH1 and VL1 comprise a variable heavy domain comprising a vhCDR1 comprising the amino acid sequence of SEQ ID NO: 142, a vhCDR2 comprising the amino acid sequence of SEQ ID NO: 143, and a vhCDR3 comprising the amino acid sequence of SEQ ID NO: 144, and a variable light domain comprising a vlCDR1 comprising the amino acid sequence of SEQ ID NO: 146, a vlCDR2 comprising the amino acid sequence of SEQ ID NO: 147, and a vlCDR3 comprising the amino acid sequence of SEQ ID NO: 148. In some such antibodies, the VH1 and VL1 comprise a variable heavy domain comprising a vhCDR1 comprising the amino acid sequence of SEQ ID NO: 150, a vhCDR2 comprising the amino acid sequence of SEQ ID NO: 151, and a vhCDR3 comprising the amino acid sequence of SEQ ID NO: 152, and a variable light domain comprising a vlCDR1 comprising the amino acid sequence of SEQ ID NO: 154, a vlCDR2 comprising the amino acid sequence of SEQ ID NO: 155, and a vlCDR3 comprising the amino acid sequence of SEQ ID NO: 156. In some such antibodies, the VH1 and VL1 comprise a variable heavy domain comprising a vhCDR1 comprising the amino acid sequence of SEQ ID NO: 118, a vhCDR2 comprising the amino acid sequence of SEQ ID NO: 158, and a vhCDR3 comprising the amino acid sequence of SEQ ID NO: 120, and a variable light domain comprising a vlCDR1 comprising the amino acid sequence of SEQ ID NO: 122, a vlCDR2 comprising the amino acid sequence of SEQ ID NO: 123, and a vlCDR3 comprising the amino acid sequence of SEQ ID NO: 160. In some such antibodies, the VH1 and VL1 comprise a variable heavy domain comprising a vhCDR1 comprising the amino acid sequence of SEQ ID NO: 162, a vhCDR2 comprising the amino acid sequence of SEQ ID NO: 163, and a vhCDR3 comprising the amino acid sequence of SEQ ID NO: 164, and a variable light domain comprising a vlCDR1 comprising the amino acid sequence of SEQ ID NO: 166, a vlCDR2 comprising the amino acid sequence of SEQ ID NO: 167, and a vlCDR3 comprising the amino acid sequence of SEQ ID NO: 168. In some such antibodies, the VH1 and VL1 comprise a variable heavy domain comprising a vhCDR1 comprising the amino acid sequence of SEQ ID NO: 170, a vhCDR2 comprising the amino acid sequence of SEQ ID NO: 171, and a vhCDR3 comprising the amino acid sequence of SEQ ID NO: 172, and a variable light domain comprising a vlCDR1 comprising the amino acid sequence of SEQ ID NO: 174, a vlCDR2 comprising the amino acid sequence of SEQ ID NO: 175, and a vlCDR3 comprising the amino acid sequence of SEQ ID NO: 176.

In some such antibodies, the VH1 is at least 90%, at least 95%, at least 96%, at least 97%, at least 98%, or at least 99% identical to the amino acid sequence of any one of SEQ ID NOS: 1674, 1200, 2241, 2253, and 2259, and the VL1 is at least 90%, at least 95%, at least 96%, at least 97%, at least 98%, or at least 99% identical to the amino acid sequence of any one of SEQ ID NOS: 1204 and 2243. In some such antibodies, the VH1 is at least 90%, at least 95%, at least 96%, at least 97%, at least 98%, or at least 99% identical to the amino acid sequence of SEQ ID NO: 1674, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of SEQ ID NO: 1200, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of SEQ ID NO: 2241, and the VL1 is at least 90%, at least 95%, at least 96%, at least 97%, at least 98%, or at least 99% identical to the amino acid sequence of SEQ ID NO: 2243. In some such antibodies, the VH1 is at least 90%, at least 95%, at least 96%, at least 97%, at least 98%, or at least 99% identical to the amino acid sequence of SEQ ID NO: 2253, and the VL1 is at least 90%, at least 95%, at least 96%, at least 97%, at least 98%, or at least 99% identical to the amino acid sequence of SEQ ID NO: 2243. In some such antibodies, the VH1 is at least 90%, at least 95%, at least 96%, at least 97%, at least 98%, or at least 99% identical to the amino acid sequence of SEQ ID NO: 2259, and the VL1 is at least 90%, at least 95%, at least 96%, at least 97%, at least 98%, or at least 99% identical to the amino acid sequence of SEQ ID NO: 2243. In some such antibodies, the VH1 is at least 90%, at least 95%, at least 96%, at least 97%, at least 98%, or at least 99% identical to the amino acid sequence of any one of SEQ ID NOS: 1427-1823 and 2316-2330, and the VL1 is at least 90%, at least 95%, at least 96%, at least 97%, at least 98%, or at least 99% identical to the amino acid sequence of any one of SEQ ID NOS: 1824-1923. In some such antibodies, the VH1 is at least 90%, at least 95%, at least 96%, at least 97%, at least 98%, or at least 99% identical to the amino acid sequence of any one of SEQ ID NOS: 1674, 1200, 1661, and 2323, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of SEQ ID NO: 1661, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of SEQ ID NO: 2323, and the VL1 is at least 90%, at least 95%, at least 96%, at least 97%, at least 98%, or at least 99% identical to the amino acid sequence of SEQ ID NO: 1204. In some such antibodies, the VH1 is at least 90%, at least 95%, at least 96%, at least 97%, at least 98%, or at least 99% identical to the amino acid sequence of SEQ ID NO: 101, and the VL1 is at least 90%, at least 95%, at least 96%, at least 97%, at least 98%, or at least 99% identical to the amino acid sequence of SEQ ID NO: 105. In some such antibodies, the VH1 is at least 90%, at least 95%, at least 96%, at least 97%, at least 98%, or at least 99% identical to the amino acid sequence of SEQ ID NO: 109, and the VL1 is at least 90%, at least 95%, at least 96%, at least 97%, at least 98%, or at least 99% identical to the amino acid sequence of SEQ ID NO: 113. In some such antibodies, the VH1 is at least 90%, at least 95%, at least 96%, at least 97%, at least 98%, or at least 99% identical to the amino acid sequence of SEQ ID NO: 117, and the VL1 is at least 90%, at least 95%, at least 96%, at least 97%, at least 98%, or at least 99% identical to the amino acid sequence of SEQ ID NO: 121. In some such antibodies, the VH1 is at least 90%, at least 95%, at least 96%, at least 97%, at least 98%, or at least 99% identical to the amino acid sequence of SEQ ID NO: 125, and the VL1 is at least 90%, at least 95%, at least 96%, at least 97%, at least 98%, or at least 99% identical to the amino acid sequence of SEQ ID NO: 129. In some such antibodies, the VH1 is at least 90%, at least 95%, at least 96%, at least 97%, at least 98%, or at least 99% identical to the amino acid sequence SEQ ID NO: 133, and the VL1 is at least 90%, at least 95%, at least 96%, at least 97%, at least 98%, or at least 99% identical to the amino acid sequence of SEQ ID NO: 137. In some such antibodies, the VH1 is at least 90%, at least 95%, at least 96%, at least 97%, at least 98%, or at least 99% identical to the amino acid sequence of SEQ ID NO: 141, and the VL1 is at least 90%, at least 95%, at least 96%, at least 97%, at least 98%, or at least 99% identical to the amino acid sequence of SEQ ID NO: 145. In some such antibodies, the VH1 is at least 90%, at least 95%, at least 96%, at least 97%, at least 98%, or at least 99% identical to the amino acid sequence of SEQ ID NO: 149, and the VL1 is at least 90%, at least 95%, at least 96%, at least 97%, at least 98%, or at least 99% identical to the amino acid sequence of SEQ ID NO: 153. In some such antibodies, the VH1 is at least 90%, at least 95%, at least 96%, at least 97%, at least 98%, or at least 99% identical to the amino acid sequence of SEQ ID NO: 157, and the VL1 is at least 90%, at least 95%, at least 96%, at least 97%, at least 98%, or at least 99% identical to the amino acid sequence of SEQ ID NO: 159. In some such antibodies, the VH1 is at least 90%, at least 95%, at least 96%, at least 97%, at least 98%, or at least 99% identical to the amino acid sequence of SEQ ID NO: 161, and the VL1 is at least 90%, at least 95%, at least 96%, at least 97%, at least 98%, or at least 99% identical to the amino acid sequence of SEQ ID NO: 165. In some such antibodies, the VH1 is at least 90%, at least 95%, at least 96%, at least 97%, at least 98%, or at least 99% identical to the amino acid sequence of SEQ ID NO: 169, and the VL1 is at least 90%, at least 95%, at least 96%, at least 97%, at least 98%, or at least 99% identical to the amino acid sequence of SEQ ID NO: 173. In some such antibodies, the VH1 and the VL1 are at least 90%, at least 95%, at least 96%, at least 97%, at least 98%, or at least 99% identical to any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH1 and the VL1 are at least 90%, at least 95%, at least 96%, at least 97%, at least 98%, or at least 99% identical to any pair of anti-TL1A variable heavy domain and variable light domain in Tables 26 and 29-34.

In some such antibodies, the VH1 comprises the amino acid sequence of any one of SEQ ID NOS: 1674, 1200, 2241, 2253, and 2259, and the VL1 comprises the amino acid sequence of any one of SEQ ID NOS: 1204 and 2243. In some such antibodies, the VH1 comprises the amino acid sequence of SEQ ID NO: 1674, and the VL1 comprises the amino acid sequence of SEQ ID NO: 1204. In some such antibodies, the VH1 comprises the amino acid sequence of SEQ ID NO: 1200, and the VL1 comprises the amino acid sequence of SEQ ID NO: 1204. In some such antibodies, the VH1 comprises the amino acid sequence of SEQ ID NO: 2241, and the VL1 comprises the amino acid sequence of SEQ ID NO: 2243. In some such antibodies, the VH1 comprises the amino acid sequence of SEQ ID NO: 2253, and the VL1 comprises the amino acid sequence of SEQ ID NO: 2243. In some such antibodies, the VH1 comprises the amino acid sequence of SEQ ID NO: 2259, and the VL1 comprises the amino acid sequence of SEQ ID NO: 2243. In some such antibodies, the VH1 comprises the amino acid sequence of any one of SEQ ID NOS: 1427-1823 and 2316-2330, and the VL1 comprises the amino acid sequence of any one of SEQ ID NOS: 1824-1923. In some such antibodies, the VH1 comprises the amino acid sequence of any one of SEQ ID NOS: 1674, 1200, 1661, and 2323, and the VL1 comprises the amino acid sequence of SEQ ID NO: 1204. In some such antibodies, the VH1 comprises the amino acid sequence of SEQ ID NO: 1661, and the VL1 comprises the amino acid sequence of SEQ ID NO: 1204. In some such antibodies, the VH1 comprises the amino acid sequence of SEQ ID NO: 2323, and the VL1 comprises the amino acid sequence of SEQ ID NO: 1204. In some such antibodies, the VH1 comprises the amino acid sequence of SEQ ID NO: 101, and the VL1 comprises the amino acid sequence of SEQ ID NO: 105. In some such antibodies, the VH1 comprises the amino acid sequence of SEQ ID NO: 109, and the VL1 comprises the amino acid sequence of SEQ ID NO: 113. In some such antibodies, the VH1 comprises the amino acid sequence of SEQ ID NO: 117, and the VL1 comprises the amino acid sequence of SEQ ID NO: 121. In some such antibodies, the VH1 comprises the amino acid sequence of SEQ ID NO: 125, and the VL1 comprises the amino acid sequence of SEQ ID NO: 129. In some such antibodies, the VH1 comprises the amino acid sequence of SEQ ID NO: 133, and the VL1 comprises the amino acid sequence of SEQ ID NO: 137. In some such antibodies, the VH1 comprises the amino acid sequence of SEQ ID NO: 141, and the VL1 comprises the amino acid sequence of SEQ ID NO: 145. In some such antibodies, the VH1 comprises the amino acid sequence of SEQ ID NO: 149, and the VL1 comprises the amino acid sequence of SEQ ID NO: 153. In some such antibodies, the VH1 comprises the amino acid sequence of SEQ ID NO: 157, and the VL1 comprises the amino acid sequence of SEQ ID NO: 159. In some such antibodies, the VH1 comprises the amino acid sequence of SEQ ID NO: 161, and the VL1 comprises the amino acid sequence of SEQ ID NO: 165. In some such antibodies, the VH1 comprises the amino acid sequence of SEQ ID NO: 169, and the VL1 comprises the amino acid sequence of SEQ ID NO: 173. In some such antibodies, the VH1 and the VL1 comprise any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH1 and the VL1 comprise any pair of anti-TL1A variable heavy domain and variable light domain in Tables 26 and 29-34. In some such antibodies, the VH1 or VL1 can comprise variants of any of the above in which an N-terminal glutamine or glutamate is replaced with pyroglutamate.

In some such antibodies, the VH2 and VL2 comprise a variable heavy domain comprising a vhCDR1, a vhCDR2, and a vhCDR3 contained within a variable heavy domain comprising the amino acid sequence of any one of SEQ ID NOS: 179, 2233, and 2235, and a variable light domain comprising a vlCDR1, a vlCDR2, and a vlCDR3 contained within a variable light domain comprising the amino acid sequence of any one of SEQ ID NOS: 1248, 2234, and 2237. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 179, and a variable light domain comprising a vlCDR1, a vlCDR2, and a vlCDR3 contained within a variable light domain comprising the amino acid sequence of SEQ ID NO: 1248. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 2233, and a variable light domain comprising a vlCDR1, a vlCDR2, and a vlCDR3 contained within a variable light domain comprising the amino acid sequence of SEQ ID NO: 2234. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 2235, and a variable light domain comprising a vlCDR1, a vlCDR2, and a vlCDR3 contained within a variable light domain comprising the amino acid sequence of SEQ ID NO: 2237. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 2113, and a variable light domain comprising a vlCDR1, a vlCDR2, and a vlCDR3 contained within a variable light domain comprising the amino acid sequence of SEQ ID NO: 2231. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 1967, and a variable light domain comprising a vlCDR1, a vlCDR2, and a vlCDR3 contained within a variable light domain comprising the amino acid sequence of SEQ ID NO: 1248. In some such antibodies, the VH2 and VL2 comprise a variable heavy domain comprising a vhCDR1, a vhCDR2, and a vhCDR3 contained within a variable heavy domain comprising the amino acid sequence of any one of SEQ ID NOS: 1927-2132, and a variable light domain comprising a vlCDR1, a vlCDR2, and a vlCDR3 contained within a variable light domain comprising the amino acid sequence of any one of SEQ ID NOS: 2133-2232. In some such antibodies, the VH2 and VL2 comprise a variable heavy domain comprising a vhCDR1, a vhCDR2, and a vhCDR3 contained within a variable heavy domain comprising the amino acid sequence of SEQ ID NO: 179, and a variable light domain comprising a vlCDR1, a vlCDR2, and a vlCDR3 contained within a variable light domain comprising the amino acid sequence of SEQ ID NO: 183. In some such antibodies, the VH2 and VL2 comprise a variable heavy domain comprising a vhCDR1, a vhCDR2, and a vhCDR3 and a variable light domain comprising a vlCDR1, a vlCDR2, and a vlCDR3 contained within any pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH2 and VL2 comprise a variable heavy domain comprising a vhCDR1, a vhCDR2, and a vhCDR3 and a variable light domain comprising a vlCDR1, a vlCDR2, and a vlCDR3 contained within any pair of anti-IL23 variable heavy domain and variable light domain in Tables 26 and 29-34. Optionally, the CDRs in any of the above antibodies are as defined by the Kabat, Chothia, Kabat+Chothia, AbM, contact, or IMGT definition.

In some such antibodies, the VH2 and VL2 comprise a variable heavy domain comprising a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182 or 2236, and a variable light domain comprising a vlCDR1 comprising the amino acid sequence of SEQ ID NO: 184, a vlCDR2 comprising the amino acid sequence of SEQ ID NO: 1249 or 2238, and a vlCDR3 comprising the amino acid sequence of SEQ ID NO: 1250. In some such antibodies, the VH2 and VL2 comprise a variable heavy domain comprising a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and a variable light domain comprising a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH2 and VL2 comprise a variable heavy domain comprising a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 2236, and a variable light domain comprising a vlCDR1 comprising the amino acid sequence of SEQ ID NO: 184, a vlCDR2 comprising the amino acid sequence of SEQ ID NO: 2238, and a vlCDR3 comprising the amino acid sequence of SEQ ID NO: 1250. In some such antibodies, the VH2 and VL2 comprise a variable heavy domain comprising a vhCDR1 comprising the amino acid sequence of SEQ ID NO: 2239, a vhCDR2 comprising the amino acid sequence of SEQ ID NO: 181, and a vhCDR3 comprising the amino acid sequence of SEQ ID NO: 182, and a variable light domain comprising a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH2 and VL2 comprise a variable heavy domain comprising a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and a variable light domain comprising a vlCDR1 comprising the amino acid sequence of SEQ ID NO: 184, a vlCDR2 comprising the amino acid sequence of SEQ ID NO: 115, and a vlCDR3 comprising the amino acid sequence of SEQ ID NO: 185.

In some such antibodies, the VH2 is at least 90%, at least 95%, at least 96%, at least 97%, at least 98%, or at least 99% identical to the amino acid sequence of any one of SEQ ID NOS: 179, 2233, and 2235, and the VL2 is at least 90%, at least 95%, at least 96%, at least 97%, at least 98%, or at least 99% identical to the amino acid sequence of any one of SEQ ID NOS: 1248, 2234, and 2237. In some such antibodies, the VH2 is at least 90%, at least 95%, at least 96%, at least 97%, at least 98%, or at least 99% identical to the amino acid sequence of SEQ ID NO: 179, and the VL2 is at least 90%, at least 95%, at least 96%, at least 97%, at least 98%, or at least 99% identical to the amino acid sequence of SEQ ID NO: 1248. In some such antibodies, the VH2 is at least 90%, at least 95%, at least 96%, at least 97%, at least 98%, or at least 99% identical to the amino acid sequence of SEQ ID NO: 2233, and the VL2 is at least 90%, at least 95%, at least 96%, at least 97%, at least 98%, or at least 99% identical to the amino acid sequence of SEQ ID NO: 2234. In some such antibodies, the VH2 is at least 90%, at least 95%, at least 96%, at least 97%, at least 98%, or at least 99% identical to the amino acid sequence of SEQ ID NO: 2235, and the VL2 is at least 90%, at least 95%, at least 96%, at least 97%, at least 98%, or at least 99% identical to the amino acid sequence of SEQ ID NO: 2237. In some such antibodies, the VH2 is at least 90%, at least 95%, at least 96%, at least 97%, at least 98%, or at least 99% identical to the amino acid sequence of SEQ ID NO: 2113, and the VL2 is at least 90%, at least 95%, at least 96%, at least 97%, at least 98%, or at least 99% identical to the amino acid sequence of SEQ ID NO: 2231. In some such antibodies, the VH2 is at least 90%, at least 95%, at least 96%, at least 97%, at least 98%, or at least 99% identical to the amino acid sequence of SEQ ID NO: 1967, and the VL2 is at least 90%, at least 95%, at least 96%, at least 97%, at least 98%, or at least 99% identical to the amino acid sequence of SEQ ID NO: 1248. In some such antibodies, the VH2 is at least 90%, at least 95%, at least 96%, at least 97%, at least 98%, or at least 99% identical to the amino acid sequence of any one of SEQ ID NOS: 1927-2132, and the VL2 is at least 90%, at least 95%, at least 96%, at least 97%, at least 98%, or at least 99% identical to the amino acid sequence of any one of SEQ ID NOS: 2133-2232. In some such antibodies, the VH2 is at least 90%, at least 95%, at least 96%, at least 97%, at least 98%, or at least 99% identical to the amino acid sequence of SEQ ID NO: 179, and the VL2 is at least 90%, at least 95%, at least 96%, at least 97%, at least 98%, or at least 99% identical to the amino acid sequence of SEQ ID NO: 183. In some such antibodies, the VH2 and the VL2 are at least 90%, at least 95%, at least 96%, at least 97%, at least 98%, or at least 99% identical to any pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH2 and the VL2 are at least 90%, at least 95%, at least 96%, at least 97%, at least 98%, or at least 99% identical to any pair of anti-IL23 variable heavy domain and variable light domain in Tables 26 and 29-34.

In some such antibodies, the VH2 comprises the amino acid sequence of any one of SEQ ID NOS: 179, 2233, and 2235, and the VL2 comprises the amino acid sequence of any one of SEQ ID NOS: 1248, 2234, and 2237. In some such antibodies, the VH2 comprises the amino acid sequence of SEQ ID NO: 179, and the VL2 comprises the amino acid sequence of SEQ ID NO: 1248. In some such antibodies, the VH2 comprises the amino acid sequence of SEQ ID NO: 2233, and the VL2 comprises the amino acid sequence of SEQ ID NO: 2234. In some such antibodies, the VH2 comprises the amino acid sequence of SEQ ID NO: 2235, and the VL2 comprises the amino acid sequence of SEQ ID NO: 2237. In some such antibodies, the VH2 comprises the amino acid sequence of SEQ ID NO: 2113, and the VL2 comprises the amino acid sequence of SEQ ID NO: 2231. In some such antibodies, the VH2 comprises the amino acid sequence of SEQ ID NO: 1967, and the VL2 comprises the amino acid sequence of SEQ ID NO: 1248. In some such antibodies, the VH2 comprises the amino acid sequence of any one of SEQ ID NOS: 1927-2132, and the VL2 comprises the amino acid sequence of any one of SEQ ID NOS: 2133-2232. In some such antibodies, the VH2 comprises the amino acid sequence of SEQ ID NO: 179, and the VL2 comprises the amino acid sequence of SEQ ID NO: 183. In some such antibodies, the VH2 and the VL2 comprise any pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the VH2 and the VL2 comprise any pair of anti-IL23 variable heavy domain and variable light domain in Tables 26 and 29-34. In some such antibodies, the VH2 or VL2 can comprise variants of any of the above in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of any one of SEQ ID NOS: 1674, 1200, 2241, 2253, and 2259, (ii) the VL1 comprises a vlCDR1, a vlCDR2, and a vlCDR3 contained within a variable light domain comprising the amino acid sequence of any one of SEQ ID NOS: 1204 and 2243, (iii) the VH2 comprises a vhCDR1, a vhCDR2, and a vhCDR3 contained within a variable heavy domain comprising the amino acid sequence of any one of SEQ ID NOS: 179, 2233, and 2235, and (iv) the VL2 comprises a vlCDR1, a vlCDR2, and a vlCDR3 contained within a variable light domain comprising the amino acid sequence of any one of SEQ ID NOS: 1248, 2234, and 2237, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102 or 1201, a vhCDR2 comprising the amino acid sequence of SEQ ID NO: 1924 or 1202, and a vhCDR3 comprising the amino acid sequence of SEQ ID NO: 1203 or 104, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182 or 2236, and (iv) the VL2 comprises a vlCDR1 comprising the amino acid sequence of SEQ ID NO: 184, a vlCDR2 comprising the amino acid sequence of SEQ ID NO: 1249 or 2238, and a vlCDR3 comprising the amino acid sequence of SEQ ID NO: 1250. In some such antibodies, the VH1 comprises the amino acid sequence of any one of SEQ ID NOS: 1674, 1200, 2241, 2253, and 2259, (ii) the VL1 comprises the amino acid sequence of any one of SEQ ID NOS: 1204 and 2243, (iii) the VH2 comprises the amino acid sequence of any one of SEQ ID NOS: 179, 2233, and 2235, and (iv) the VL2 comprises the amino acid sequence of any one of SEQ ID NOS: 1248, 2234, and 2237, or variants thereof in which an N-terminal glutamine or glutamate is replaced with pyroglutamate. In some such antibodies, the bispecific antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the bispecific antibody specifically binds human TL1A with a K_{D} of less than about 3E-11 M, less than about 2.9E-11 M, less than about 2.8E-11 M, or less than about 2.7E-11 M. In some such antibodies, the bispecific antibody inhibits TL1A activity in a TL1A luciferase reporter assay with an IC₅₀ of less than about 4.5 nM. In some such antibodies, the bispecific antibody specifically binds human IL23 with a K_{D} of less than about 1.1E-10 M as measured by surface plasmon resonance or Biacore at 25° C., optionally wherein the bispecific antibody specifically binds human IL23 with a K_{D} of less than about 6E-11 M, less than about 5.9E-11 M, less than about 5.8E-11 M, or less than about 5.7E-11 M. In some such antibodies, the bispecific antibody inhibits IL23 activity in an IL23 luciferase reporter assay with an IC₅₀ of less than about 65 pM, less than about 64 pM, or less than about 63 pM, optionally wherein the bispecific antibody inhibits TL23 activity with an IC₅₀ of less than about 60 pM, less than about 59 pM, less than about 58 pM, less than about 57 pM, or less than about 56 pM.

In some such antibodies, the VH1 comprises a vhCDR1, a vhCDR2, and a vhCDR3 contained within a variable heavy domain comprising the amino acid sequence of SEQ ID NO: 1674 or 1200, (ii) the VL1 comprises a vlCDR1, a vlCDR2, and a vlCDR3 contained within a variable light domain comprising the amino acid sequence of SEQ ID NO: 1204, (iii) the VH2 comprises a vhCDR1, a vhCDR2, and a vhCDR3 contained within a variable heavy domain comprising the amino acid sequence of SEQ ID NO: 179, and (iv) the VL2 comprises a vlCDR1, a vlCDR2, and a vlCDR3 contained within a variable light domain comprising the amino acid sequence of SEQ ID NO: 1248, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102 or 1201, a vhCDR2 comprising the amino acid sequence of SEQ ID NO: 1924 or 1202, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence of SEQ ID NO: 1674 or 1200, (ii) the VL1 comprises the amino acid sequence of SEQ ID NO: 1204, (iii) the VH2 comprises the amino acid sequence of SEQ ID NO: 179, and (iv) the VL2 comprises the amino acid sequence of SEQ ID NO: 1248, or variants thereof in which an N-terminal glutamine or glutamate is replaced with pyroglutamate. In some such antibodies, the bispecific antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the bispecific antibody specifically binds human TL1A with a K_{D} of less than about 3E-11 M, less than about 2.9E-11 M, less than about 2.8E-11 M, or less than about 2.7E-11 M. In some such antibodies, the bispecific antibody inhibits TL1A activity in a TL1A luciferase reporter assay with an IC₅₀ of less than about 4.5 nM. In some such antibodies, the bispecific antibody specifically binds human IL23 with a K_{D} of less than about 1.1E-10 M as measured by surface plasmon resonance or Biacore at 25° C., optionally wherein the bispecific antibody specifically binds human IL23 with a K_{D} of less than about 6E-11 M, less than about 5.9E-11 M, less than about 5.8E-11 M, or less than about 5.7E-11 M. In some such antibodies, the bispecific antibody inhibits IL23 activity in an IL23 luciferase reporter assay with an IC₅₀ of less than about 65 pM, less than about 64 pM, or less than about 63 pM, optionally wherein the bispecific antibody inhibits IL23 activity with an IC₅₀ of less than about 60 pM, less than about 59 pM, less than about 58 pM, less than about 57 pM, or less than about 56 pM.

In some such antibodies, the VH1 comprises a vhCDR1, a vhCDR2, and a vhCDR3 contained within a variable heavy domain comprising the amino acid sequence of SEQ ID NO: 1674, (ii) the VL1 comprises a vlCDR1, a vlCDR2, and a vlCDR3 contained within a variable light domain comprising the amino acid sequence of SEQ ID NO: 1204, (iii) the VH2 comprises a vhCDR1, a vhCDR2, and a vhCDR3 contained within a variable heavy domain comprising the amino acid sequence of SEQ ID NO: 179, and (iv) the VL2 comprises a vlCDR1, a vlCDR2, and a vlCDR3 contained within a variable light domain comprising the amino acid sequence of SEQ ID NO: 1248, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence of SEQ ID NO: 1674, (ii) the VL1 comprises the amino acid sequence of SEQ ID NO: 1204, (iii) the VH2 comprises the amino acid sequence of SEQ ID NO: 179, and (iv) the VL2 comprises the amino acid sequence of SEQ ID NO: 1248, or variants thereof in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of SEQ ID NO: 1200, (ii) the VL1 comprises a vlCDR1, a vlCDR2, and a vlCDR3 contained within a variable light domain comprising the amino acid sequence of SEQ ID NO: 1204, (iii) the VH2 comprises a vhCDR1, a vhCDR2, and a vhCDR3 contained within a variable heavy domain comprising the amino acid sequence of SEQ ID NO: 179, and (iv) the VL2 comprises a vlCDR1, a vlCDR2, and a vlCDR3 contained within a variable light domain comprising the amino acid sequence of SEQ ID NO: 1248, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence SEQ ID NO: 1200, (ii) the VL1 comprises the amino acid sequence of SEQ ID NO: 1204, (iii) the VH2 comprises the amino acid sequence of SEQ ID NO: 179, and (iv) the VL2 comprises the amino acid sequence of SEQ ID NO: 1248, or variants thereof in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of SEQ ID NO: 2241, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence of SEQ ID NO: 2241, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234, or variants thereof in which an N-terminal glutamine or glutamate is replaced with pyroglutamate. In some such antibodies, the bispecific antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the bispecific antibody specifically binds human TL1A with a K_{D} of less than about 3E-11 M, less than about 2.9E-11 M, less than about 2.8E-11 M, or less than about 2.7E-11 M. In some such antibodies, the bispecific antibody inhibits TL1A activity in a TL1A luciferase reporter assay with an IC₅₀ of less than about 4.5 nM. In some such antibodies, the bispecific antibody specifically binds human IL23 with a K_{D} of less than about 1.1E-10 M as measured by surface plasmon resonance or Biacore at 25° C., optionally wherein the bispecific antibody specifically binds human IL23 with a K_{D} of less than about 6E-11 M, less than about 5.9E-11 M, less than about 5.8E-11 M, or less than about 5.7E-11 M. In some such antibodies, the bispecific antibody inhibits IL23 activity in an IL23 luciferase reporter assay with an IC₅₀ of less than about 65 pM, less than about 64 pM, or less than about 63 pM, optionally wherein the bispecific antibody inhibits IL23 activity with an IC₅₀ of less than about 60 pM, less than about 59 pM, less than about 58 pM, less than about 57 pM, or less than about 56 pM.

In some such antibodies, the VH1 comprises a vhCDR1, a vhCDR2, and a vhCDR3 contained within a variable heavy domain comprising the amino acid sequence of SEQ ID NO: 2241, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2235, and (iv) the VL2 comprises a vlCDR1, a vlCDR2, and a vlCDR3 contained within a variable light domain comprising the amino acid sequence of SEQ ID NO: 2237, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 2236, and (iv) the VL2 comprises a vlCDR1 comprising the amino acid sequence of SEQ ID NO: 184, a vlCDR2 comprising the amino acid sequence of SEQ ID NO: 2238, and a vlCDR3 comprising the amino acid sequence of SEQ ID NO: 1250. In some such antibodies, the VH1 comprises the amino acid sequence of SEQ ID NO: 2241, (ii) the VL1 comprises the amino acid sequence of SEQ ID NO: 2243, (iii) the VH2 comprises the amino acid sequence of SEQ ID NO: 2235, and (iv) the VL2 comprises the amino acid sequence of SEQ ID NO: 2237, or variants thereof in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of SEQ ID NO: 2253, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence of SEQ ID NO: 2253, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234, or variants thereof in which an N-terminal glutamine or glutamate is replaced with pyroglutamate.

In some such antibodies, the VH1 comprises a vhCDR1, a vhCDR2, and a vhCDR3 contained within a variable heavy domain comprising the amino acid sequence of SEQ ID NO: 2259, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234, optionally wherein the CDRs are as defined by the Kabat, Chothia, Kabat+Chothia, AbM, contact, or IMGT definition. In some such antibodies, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 104, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the VH1 comprises the amino acid sequence of SEQ ID NO: 2259, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234, or variants thereof in which an N-terminal glutamine or glutamate is replaced with pyroglutamate.

In some such antibodies, the first Fc domain and the second Fc domain comprise a set of heterodimerization skew variants. In some such antibodies, the first Fc domain and the second Fc domain each comprise one or more ablation variants. In some such antibodies, the first monomer or the second monomer comprises one or more pI variants. In some such antibodies, the first heavy chain constant domain comprises amino acid substitutions E233P/L234V/L235A/G236del/S267K and/or the second heavy chain constant domain comprises amino acid substitutions E233P/L234V/L235A/G236del/S267K, wherein numbering is according to EU numbering. In some such antibodies, the first heavy chain constant domain comprises amino acid substitutions M428L/N434S and/or the second heavy chain constant domain comprises amino acid substitutions M428L/N434S, wherein numbering is according to EU numbering. In some such antibodies, the first heavy chain constant domain comprises amino acid substitutions E233P/L234V/L235A/G236del/S267K and M428L/N434S and/or the second heavy chain constant domain comprises amino acid substitutions E233P/L234V/L235A/G236del/S267K and M428L/N434S, wherein numbering is according to EU numbering. In some such antibodies, the first heavy chain constant domain or the second heavy chain constant domain comprises amino acid substitutions L368D/K370S, wherein numbering is according to EU numbering. In some such antibodies, the first heavy chain constant domain or the second heavy chain constant domain comprises amino acid substitutions N208D/Q295E/N384D/Q418E/N421D, wherein numbering is according to EU numbering. In some such antibodies, the first heavy chain constant domain or the second heavy chain constant domain comprises amino acid substitutions E233P/L234V/L235A/G236del/S267K, M428L/N434S, L368D/K370S, and N208D/Q295E/N384D/Q418E/N421D, wherein numbering is according to EU numbering. In some such antibodies, the first heavy chain constant domain or the second heavy chain constant domain comprises amino acid substitutions E357Q/S364K, wherein numbering is according to EU numbering. In some such antibodies, the first heavy chain constant domain comprises amino acid substitutions L368D/K370S, and the second heavy chain constant domain comprises amino acid substitutions E357Q/S364K, wherein numbering is according to EU numbering. In some such antibodies, the first heavy chain constant domain or the second heavy chain constant domain comprises amino acid substitutions E233P/L234V/L235A/G236del/S267K, M428L/N434S, and E357Q/S364K, wherein numbering is according to EU numbering. In some such antibodies, the first heavy chain constant domain comprises amino acid substitutions E233P/L234V/L235A/G236del/S267K, M428L/N434S, L368D/K370S, and N208D/Q295E/N384D/Q418E/N421D, wherein numbering is according to EU numbering, and the second heavy chain constant domain comprises amino acid substitutions E233P/L234V/L235A/G236del/S267K, M428L/N434S, and E357Q/S364K, wherein numbering is according to EU numbering.

In some such antibodies, the first heavy chain constant domain is of isotype IgG1, and the second heavy chain constant domain is of isotype IgG1. In some such antibodies, the bispecific antibody is a chimeric or humanized bispecific antibody.

In some such antibodies, the first heavy chain comprises the amino acid sequence of SEQ ID NO: 2264 or a variant thereof in which the C-terminal lysine or the C-terminal lysine and glycine are removed, and the first light chain comprises the amino acid sequence of SEQ ID NO: 2265 or a variant thereof in which the N-terminal glutamine is replaced with pyroglutamate. In some such antibodies, the first heavy chain comprises the amino acid sequence of SEQ ID NO: 2264 or a variant thereof in which the C-terminal lysine or the C-terminal lysine and glycine are removed, and the first light chain comprises the amino acid sequence of SEQ ID NO: 2269 or a variant thereof in which the N-terminal glutamine is replaced with pyroglutamate. In some such antibodies, the first heavy chain is at least 90%, at least 95%, at least 96%, at least 97%, at least 98%, or at least 99% identical to the amino acid sequence of SEQ ID NO: 2264, and the first light chain is at least 90%, at least 95%, at least 96%, at least 97%, at least 98%, or at least 99% identical to the amino acid sequence of SEQ ID NO: 2265. In some such antibodies, the first heavy chain is at least 90%, at least 95%, at least 96%, at least 97%, at least 98%, or at least 99% identical to the amino acid sequence of SEQ ID NO: 2264, and the first light chain is at least 90%, at least 95%, at least 96%, at least 97%, at least 98%, or at least 99% identical to the amino acid sequence of SEQ ID NO: 2269. In some such antibodies, the second heavy chain comprises the amino acid sequence of SEQ ID NO: 2266 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2267. In some such antibodies, the second heavy chain is at least 90%, at least 95%, at least 96%, at least 97%, at least 98%, or at least 99% identical to the amino acid sequence of SEQ ID NO: 2266, and the second light chain is at least 90%, at least 95%, at least 96%, at least 97%, at least 98%, or at least 99% identical to the amino acid sequence of SEQ ID NO: 2267.

In some such antibodies, the first heavy chain, the first light chain, the second heavy chain, and the second light chain comprise any set of four chains from Table 31 or 32. In some such antibodies, the first heavy chain comprises the amino acid sequence of SEQ ID NO: 2264 or a variant thereof in which the C-terminal lysine or the C-terminal lysine and glycine are removed, the first light chain comprises the amino acid sequence of SEQ ID NO: 2265 or a variant thereof in which the N-terminal glutamine is replaced with pyroglutamate, the second heavy chain comprises the amino acid sequence of SEQ ID NO: 2266 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2267. In some such antibodies, the first heavy chain comprises the amino acid sequence of SEQ ID NO: 2264 or a variant thereof in which the C-terminal lysine or the C-terminal lysine and glycine are removed, the first light chain comprises the amino acid sequence of SEQ ID NO: 2269 or a variant thereof in which the N-terminal glutamine is replaced with pyroglutamate, the second heavy chain comprises the amino acid sequence of SEQ ID NO: 2266 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2267. In some such antibodies, the first heavy chain is at least 90%, at least 95%, at least 96%, at least 97%, at least 98%, or at least 99% identical to the amino acid sequence of SEQ ID NO: 2264, the first light chain is at least 90%, at least 95%, at least 96%, at least 97%, at least 98%, or at least 99% identical to the amino acid sequence of SEQ ID NO: 2265, the second heavy chain is at least 90%, at least 95%, at least 96%, at least 97%, at least 98%, or at least 99% identical to the amino acid sequence of SEQ ID NO: 2266, and the second light chain is at least 90%, at least 95%, at least 96%, at least 97%, at least 98%, or at least 99% identical to the amino acid sequence of SEQ ID NO: 2267. In some such antibodies, the first heavy chain is at least 90%, at least 95%, at least 96%, at least 97%, at least 98%, or at least 99% identical to the amino acid sequence of SEQ ID NO: 2264, the first light chain is at least 90%, at least 95%, at least 96%, at least 97%, at least 98%, or at least 99% identical to the amino acid sequence of SEQ ID NO: 2269, the second heavy chain is at least 90%, at least 95%, at least 96%, at least 97%, at least 98%, or at least 99% identical to the amino acid sequence of SEQ ID NO: 2266, and the second light chain is at least 90%, at least 95%, at least 96%, at least 97%, at least 98%, or at least 99% identical to the amino acid sequence of SEQ ID NO: 2267.

In some such antibodies, the bispecific antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the bispecific antibody specifically binds human TL1A with a K_{D} of less than about 3E-11 M, less than about 2.9E-11 M, less than about 2.8E-11 M, or less than about 2.7E-11 M. In some such antibodies, the bispecific antibody inhibits TL1A activity in a TL1A luciferase reporter assay with an IC₅₀ of less than about 4.5 nM. In some such antibodies, the bispecific antibody specifically binds human IL23 with a K_{D} of less than about 1.1E-10 M as measured by surface plasmon resonance or Biacore at 25° C., optionally wherein the bispecific antibody specifically binds human IL23 with a K_{D} of less than about 6E-11 M, less than about 5.9E-11 M, less than about 5.8E-11 M, or less than about 5.7E-11 M. In some such antibodies, the bispecific antibody inhibits IL23 activity in an IL23 luciferase reporter assay with an IC₅₀ of less than about 65 pM, less than about 64 pM, or less than about 63 pM, optionally wherein the bispecific antibody inhibits IL23 activity with an IC₅₀ of less than about 60 pM, less than about 59 pM, less than about 58 pM, less than about 57 pM, or less than about 56 pM.

In another aspect, provided is a bispecific antibody comprising: (a) a means for binding TL1A; and (b) a means for binding IL23. In another aspect, provided is a pharmaceutical composition comprising: (a) the above bispecific antibody; and (b) a pharmaceutically acceptable carrier. In another aspect, provided is a pharmaceutical composition comprising: (a) a first composition comprising a means for binding TL1A; and (b) a second composition comprising a means for binding IL23. In another aspect, provided is a pharmaceutical composition comprising: (a) a means for binding TL1A; (b) a means for binding IL23; and (c) a pharmaceutically acceptable carrier.

In another aspect, provided are pharmaceutical compositions comprising any of the above bispecific antibodies and a pharmaceutically acceptable carrier. In another aspect, provided are kits comprising any of the above bispecific antibodies or pharmaceutical compositions. In another aspect, provided are vessels or delivery devices comprising any of the above bispecific antibodies or pharmaceutical compositions. In another aspect, provided are nucleic acid compositions comprising a nucleic acid or a set of nucleic acids encoding any of the above bispecific antibodies. In another aspect, provided are expression vector compositions comprising an expression vector or a set of expression vectors comprising the above nucleic acid composition. In another aspect, provided are host cells comprising the expression vector composition. In another aspect, provided are methods of making a bispecific antibody, comprising culturing the host cell under conditions wherein the bispecific antibody is expressed, and recovering the bispecific antibody.

In another aspect, provided are methods of inhibiting or reducing TL1A-mediated activity and/or IL23-mediated activity in a subject in need thereof, comprising administering to the subject any of the above bispecific antibodies or pharmaceutical compositions. In another aspect, provided are any of the above bispecific antibodies or pharmaceutical compositions for use in inhibiting or reducing TL1A-mediated activity and/or IL23-mediated activity in a subject in need thereof. In another aspect, provided are uses of any of the above bispecific antibodies or pharmaceutical compositions for inhibiting or reducing TL1A-mediated activity and/or IL23-mediated activity in a subject in need thereof. In another aspect, provided are uses of any of the above bispecific antibodies or pharmaceutical compositions in the manufacture of a medicament for inhibiting or reducing TL1A-mediated activity and/or IL23-mediated activity in a subject in need thereof.

In another aspect, provided are methods of treating a TL1A-associated disease and/or IL23-associated disease in a subject in need thereof, comprising administering to the subject any of the above bispecific antibodies or pharmaceutical compositions. In another aspect, provided are any of the above bispecific antibodies or pharmaceutical compositions for use in the treatment of a TL1A-associated disease and/or IL23-associated disease in a subject in need thereof. In another aspect, provided are uses of any of the above bispecific antibodies or pharmaceutical compositions for the treatment of a TL1A-associated disease and/or IL23-associated disease in a subject in need thereof. In another aspect, provided are uses of any of the above bispecific antibodies or pharmaceutical compositions in the manufacture of a medicament for the treatment of a TL1A-associated disease and/or IL23-associated disease in a subject in need thereof. In some such methods, antibodies or compositions for use, or uses, the TL1A-associated disease and/or IL23-associated disease is an inflammatory disease, an autoimmune disease, or a fibrotic disease, optionally wherein the TL1A-associated disease and/or IL23-associated disease is an inflammatory bowel disease. In some such methods, antibodies or compositions for use, or uses, the subject is a human.

In another aspect, provided are antibodies comprising a TNF-like ligand 1A (TL1A) antigen-binding domain. In some such antibodies, the antibody comprises a variable heavy domain comprising a vhCDR1, a vhCDR2, and a vhCDR3 contained within a variable heavy domain comprising the amino acid sequence of any one of SEQ ID NOS: 2241, 2253, 2259, 1674, and 1200, and a variable light domain comprising a vlCDR1, a vlCDR2, and a vlCDR3 contained within a variable light domain comprising the amino acid sequence of any one of SEQ ID NOS: 2243 and 1204. In some such antibodies, the antibody comprises a variable heavy domain comprising a vhCDR1, a vhCDR2, and a vhCDR3 contained within a variable heavy domain comprising the amino acid sequence of SEQ ID NO: 2241, and a variable light domain comprising a vlCDR1, a vlCDR2, and a vlCDR3 contained within a variable light domain comprising the amino acid sequence of SEQ ID NO: 2243. In some such antibodies, the antibody comprises a variable heavy domain comprising a vhCDR1, a vhCDR2, and a vhCDR3 contained within a variable heavy domain comprising the amino acid sequence of SEQ ID NO: 2253, and a variable light domain comprising a vlCDR1, a vlCDR2, and a vlCDR3 contained within a variable light domain comprising the amino acid sequence of SEQ ID NO: 2243. In some such antibodies, the antibody comprises a variable heavy domain comprising a vhCDR1, a vhCDR2, and a vhCDR3 contained within a variable heavy domain comprising the amino acid sequence of SEQ ID NO: 2259, and a variable light domain comprising a vlCDR1, a vlCDR2, and a vlCDR3 contained within a variable light domain comprising the amino acid sequence of SEQ ID NO: 2243. In some such antibodies, the antibody comprises a variable heavy domain comprising a vhCDR1, a vhCDR2, and a vhCDR3 contained within a variable heavy domain comprising the amino acid sequence of SEQ ID NO: 1674, and a variable light domain comprising a vlCDR1, a vlCDR2, and a vlCDR3 contained within a variable light domain comprising the amino acid sequence of SEQ ID NO: 1204. In some such antibodies, the antibody comprises a variable heavy domain comprising a vhCDR1, a vhCDR2, and a vhCDR3 contained within a variable heavy domain comprising the amino acid sequence of SEQ ID NO: 1200, and a variable light domain comprising a vlCDR1, a vlCDR2, and a vlCDR3 contained within a variable light domain comprising the amino acid sequence of SEQ ID NO: 1204. In some such antibodies, the antibody comprises a variable heavy domain comprising a vhCDR1, a vhCDR2, and a vhCDR3 contained within a variable heavy domain comprising the amino acid sequence of any one of SEQ ID NOS: 1674, 1200, 1661, and 2323, and a variable light domain comprising a vlCDR1, a vlCDR2, and a vlCDR3 contained within a variable light domain comprising the amino acid sequence of SEQ ID NO: 1204. In some such antibodies, the antibody comprises a variable heavy domain comprising a vhCDR1, a vhCDR2, and a vhCDR3 contained within a variable heavy domain comprising the amino acid sequence of SEQ ID NO: 1661, and a variable light domain comprising a vlCDR1, a vlCDR2, and a vlCDR3 contained within a variable light domain comprising the amino acid sequence of SEQ ID NO: 1204. In some such antibodies, the antibody comprises a variable heavy domain comprising a vhCDR1, a vhCDR2, and a vhCDR3 contained within a variable heavy domain comprising the amino acid sequence of SEQ ID NO: 2323, and a variable light domain comprising a vlCDR1, a vlCDR2, and a vlCDR3 contained within a variable light domain comprising the amino acid sequence of SEQ ID NO: 1204. In some such antibodies, the antibody comprises a variable heavy domain comprising a vhCDR1, a vhCDR2, and a vhCDR3 and a variable light domain comprising a vlCDR1, a vlCDR2, and a vlCDR3 contained within a variable heavy domain and variable light domain pair in XENP53412, XENP53413, XENP53369, XENP53375, XENP53387, XENP53415, XENP53370, XENP53371, XENP53376, XENP53377, XENP53378, XENP53379, XENP53380, or XENP53414. Optionally, the CDRs in any of the above antibodies are as defined by the Kabat, Chothia, Kabat+Chothia, AbM, contact, or IMGT definition.

In some such antibodies, the antibody comprises a variable heavy domain comprising a vhCDR1 comprising the amino acid sequence of SEQ ID NO: 102 or 1201, a vhCDR2 comprising the amino acid sequence of SEQ ID NO: 1924 or 1202, and a vhCDR3 comprising the amino acid sequence of SEQ ID NO: 1203 or 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the antibody comprises a variable heavy domain comprising a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the antibody comprises a variable heavy domain comprising a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the antibody comprises a variable heavy domain comprising a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, the antibody comprises a variable heavy domain comprising a vhCDR1 comprising the amino acid sequence of SEQ ID NO: 102, a vhCDR2 comprising the amino acid sequence of SEQ ID NO: 1926, and a vhCDR3 comprising the amino acid sequence of SEQ ID NO: 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108.

In some such antibodies, the variable heavy domain is at least 90%, at least 95%, at least 96%, at least 97%, at least 98%, or at least 99% identical to the amino acid sequence of any one of SEQ ID NOS: 2241, 2253, 2259, 1674, and 1200, and the variable light domain is at least 90%, at least 95%, at least 96%, at least 97%, at least 98%, or at least 99% identical to the amino acid sequence of any one of SEQ ID NOS: 2243 and 1204. In some such antibodies, the variable heavy domain is at least 90%, at least 95%, at least 96%, at least 97%, at least 98%, or at least 99% identical to the amino acid sequence of SEQ ID NO: 2241, and the variable light domain is at least 90%, at least 95%, at least 96%, at least 97%, at least 98%, or at least 99% identical to the amino acid sequence of SEQ ID NO: 2243. In some such antibodies, the variable heavy domain is at least 90%, at least 95%, at least 96%, at least 97%, at least 98%, or at least 99% identical to the amino acid sequence of SEQ ID NO: 2253, and the variable light domain is at least 90%, at least 95%, at least 96%, at least 97%, at least 98%, or at least 99% identical to the amino acid sequence of SEQ ID NO: 2243. In some such antibodies, the variable heavy domain is at least 90%, at least 95%, at least 96%, at least 97%, at least 98%, or at least 99% identical to the amino acid sequence of SEQ ID NO: 2259, and the variable light domain is at least 90%, at least 95%, at least 96%, at least 97%, at least 98%, or at least 99% identical to the amino acid sequence of SEQ ID NO: 2243. In some such antibodies, the variable heavy domain is at least 90%, at least 95%, at least 96%, at least 97%, at least 98%, or at least 99% identical to the amino acid sequence of SEQ ID NO: 1674, and the variable light domain is at least 90%, at least 95%, at least 96%, at least 97%, at least 98%, or at least 99% identical to the amino acid sequence of SEQ ID NO: 1204. In some such antibodies, variable heavy domain is at least 90%, at least 95%, at least 96%, at least 97%, at least 98%, or at least 99% identical to the amino acid sequence of SEQ ID NO: 1200, and the variable light domain is at least 90%, at least 95%, at least 96%, at least 97%, at least 98%, or at least 99% identical to the amino acid sequence of SEQ ID NO: 1204. In some such antibodies, the variable heavy domain is at least 90%, at least 95%, at least 96%, at least 97%, at least 98%, or at least 99% identical to the amino acid sequence of any one of SEQ ID NOS: 1427-1823 and 2316-2330, and the variable light domain is at least 90%, at least 95%, at least 96%, at least 97%, at least 98%, or at least 99% identical to the amino acid sequence of any one of SEQ ID NOS: 1824-1923. In some such antibodies, the variable heavy domain is at least 90%, at least 95%, at least 96%, at least 97%, at least 98%, or at least 99% identical to the amino acid sequence of any one of SEQ ID NOS: 1674, 1200, 1661, and 2323, and the variable light domain is at least 90%, at least 95%, at least 96%, at least 97%, at least 98%, or at least 99% identical to the amino acid sequence of SEQ ID NO: 1204. In some such antibodies, the variable heavy domain is at least 90%, at least 95%, at least 96%, at least 97%, at least 98%, or at least 99% identical to the amino acid sequence of SEQ ID NO: 1661, and the variable light domain is at least 90%, at least 95%, at least 96%, at least 97%, at least 98%, or at least 99% identical to the amino acid sequence of SEQ ID NO: 1204. In some such antibodies, the variable heavy domain is at least 90%, at least 95%, at least 96%, at least 97%, at least 98%, or at least 99% identical to the amino acid sequence of SEQ ID NO: 2323, and the variable light domain is at least 90%, at least 95%, at least 96%, at least 97%, at least 98%, or at least 99% identical to the amino acid sequence of SEQ ID NO: 1204. In some such antibodies, the variable heavy domain and the variable light domain are at least 90%, at least 95%, at least 96%, at least 97%, at least 98%, or at least 99% identical to any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the variable heavy domain and the variable light domain are at least 90%, at least 95%, at least 96%, at least 97%, at least 98%, or at least 99% identical to any pair of anti-TL1A variable heavy domain and variable light domain in Table 29 or 33.

In some such antibodies, the variable heavy domain comprises the amino acid sequence of any one of SEQ ID NOS: 2241, 2253, 2259, 1674, and 1200, and the variable light domain comprises the amino acid sequence of any one of SEQ ID NOS: 2243 and 1204. In some such antibodies, the variable heavy domain comprises the amino acid sequence of SEQ ID NO: 2241, and the variable light domain comprises the amino acid sequence of SEQ ID NO: 2243. In some such antibodies, the variable heavy domain comprises the amino acid sequence of SEQ ID NO: 2253, and the variable light domain comprises the amino acid sequence of SEQ ID NO: 2243. In some such antibodies, the variable heavy domain comprises the amino acid sequence of SEQ ID NO: 2259, and the variable light domain comprises the amino acid sequence of SEQ ID NO: 2243. In some such antibodies, the variable heavy domain comprises the amino acid sequence of SEQ ID NO: 1674, and the variable light domain comprises the amino acid sequence of SEQ ID NO: 1204. In some such antibodies, the variable heavy domain comprises the amino acid sequence of SEQ ID NO: 1200, and the variable light domain comprises the amino acid sequence of SEQ ID NO: 1204. In some such antibodies, the variable heavy domain comprises the amino acid sequence of any one of SEQ ID NOS: 1427-1823 and 2316-2330, and the variable light domain comprises the amino acid sequence of any one of SEQ ID NOS: 1824-1923. In some such antibodies, the variable heavy domain comprises the amino acid sequence of any one of SEQ ID NOS: 1674, 1200, 1661, and 2323, and the variable light domain comprises the amino acid sequence of SEQ ID NO: 1204. In some such antibodies, the variable heavy domain comprises the amino acid sequence of SEQ ID NO: 1661, and the variable light domain comprises the amino acid sequence of SEQ ID NO: 1204. In some such antibodies, the variable heavy domain comprises the amino acid sequence of SEQ ID NO: 2323, and the variable light domain comprises the amino acid sequence of SEQ ID NO: 1204. In some such antibodies, the variable heavy domain and the variable light domain comprise any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the variable heavy domain and the variable light domain comprise any pair of anti-TL1A variable heavy domain and variable light domain in Table 29 or 33. In some such antibodies, the variable heavy domain or the variable light domain can comprise variants of any of the above in which an N-terminal glutamine or glutamate is replaced with pyroglutamate.

In some such antibodies, (i) the variable heavy domain comprises a vhCDR1, a vhCDR2, and a vhCDR3 contained within a variable heavy domain comprising the amino acid sequence of any one of SEQ ID NOS: 2241, 2253, 2259, 1674, and 1200, and (ii) the variable light domain comprises a vlCDR1, a vlCDR2, and a vlCDR3 contained within a variable light domain comprising the amino acid sequence of any one of SEQ ID NOS: 2243 and 1204, optionally wherein the CDRs are as defined by the Kabat, Chothia, Kabat+Chothia, AbM, contact, or IMGT definition. In some such antibodies, (i) the variable heavy domain comprises a vhCDR1 comprising the amino acid sequence of SEQ ID NO: 102 or 1201, a vhCDR2 comprising the amino acid sequence of SEQ ID NO: 1924 or 1202, and a vhCDR3 comprising the amino acid sequence of SEQ ID NO: 1203 or 104, and (ii) the variable light domain comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, (i) the variable heavy domain comprises the amino acid sequence of any one of SEQ ID NOS: 2241, 2253, 2259, 1674, and 1200, and (ii) the variable light domain comprises the amino acid sequence of any one of SEQ ID NOS: 2243 and 1204, or variants thereof in which an N-terminal glutamine or glutamate is replaced with pyroglutamate. In some such antibodies, the antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the antibody specifically binds human TL1A with a K_{D} of less than about 3E-11 M, less than about 2.9E-11 M, less than about 2.8E-11 M, or less than about 2.7E-11 M. In some such antibodies, the antibody inhibits TL1A activity in a TL1A luciferase reporter assay with an IC₅₀ of less than about 4.5 nM.

In some such antibodies, (i) the variable heavy domain comprises a vhCDR1, a vhCDR2, and a vhCDR3 contained within a variable heavy domain comprising the amino acid sequence of SEQ ID NO: 2241, and (ii) the variable light domain comprises a vlCDR1, a vlCDR2, and a vlCDR3 contained within a variable light domain comprising the amino acid sequence of SEQ ID NO: 2243, optionally wherein the CDRs are as defined by the Kabat, Chothia, Kabat+Chothia, AbM, contact, or IMGT definition. In some such antibodies, (i) the variable heavy domain comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, and (ii) the variable light domain comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, (i) the variable heavy domain comprises the amino acid sequence of SEQ ID NO: 2241, and (ii) the variable light domain comprises the amino acid sequence of SEQ ID NO: 2243, or variants thereof in which an N-terminal glutamine or glutamate is replaced with pyroglutamate. In some such antibodies, the antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the antibody specifically binds human TL1A with a K_{D} of less than about 3E-11 M, less than about 2.9E-11 M, less than about 2.8E-11 M, or less than about 2.7E-11 M. In some such antibodies, the antibody inhibits TL1A activity in a TL1A luciferase reporter assay with an IC₅₀ of less than about 4.5 nM.

In some such antibodies, (i) the variable heavy domain comprises a vhCDR1, a vhCDR2, and a vhCDR3 contained within a variable heavy domain comprising the amino acid sequence of SEQ ID NO: 2253, and (ii) the variable light domain comprises a vlCDR1, a vlCDR2, and a vlCDR3 contained within a variable light domain comprising the amino acid sequence of SEQ ID NO: 2243, optionally wherein the CDRs are as defined by the Kabat, Chothia, Kabat+Chothia, AbM, contact, or IMGT definition. In some such antibodies, (i) the variable heavy domain comprises a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, and (ii) the variable light domain comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, (i) the variable heavy domain comprises the amino acid sequence of SEQ ID NO: 2253, and (ii) the variable light domain comprises the amino acid sequence of SEQ ID NO: 2243, or variants thereof in which an N-terminal glutamine or glutamate is replaced with pyroglutamate.

In some such antibodies, (i) the variable heavy domain comprises a vhCDR1, a vhCDR2, and a vhCDR3 contained within a variable heavy domain comprising the amino acid sequence of SEQ ID NO: 2259, and (ii) the variable light domain comprises a vlCDR1, a vlCDR2, and a vlCDR3 contained within a variable light domain comprising the amino acid sequence of SEQ ID NO: 2243, optionally wherein the CDRs are as defined by the Kabat, Chothia, Kabat+Chothia, AbM, contact, or IMGT definition. In some such antibodies, (i) the variable heavy domain comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 104, and (ii) the variable light domain comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, (i) the variable heavy domain comprises the amino acid sequence of SEQ ID NO: 2259, and (ii) the variable light domain comprises the amino acid sequence of SEQ ID NO: 2243, or variants thereof in which an N-terminal glutamine or glutamate is replaced with pyroglutamate.

In some such antibodies, (i) the variable heavy domain comprises a vhCDR1, a vhCDR2, and a vhCDR3 contained within a variable heavy domain comprising the amino acid sequence of SEQ ID NO: 1674, and (ii) the variable light domain comprises a vlCDR1, a vlCDR2, and a vlCDR3 contained within a variable light domain comprising the amino acid sequence of SEQ ID NO: 1204, optionally wherein the CDRs are as defined by the Kabat, Chothia, Kabat+Chothia, AbM, contact, or IMGT definition. In some such antibodies, (i) the variable heavy domain comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, and (ii) the variable light domain comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, (i) the variable heavy domain comprises the amino acid sequence of SEQ ID NO: 1674, and (ii) the variable light domain comprises the amino acid sequence of SEQ ID NO: 1204, or variants thereof in which an N-terminal glutamine or glutamate is replaced with pyroglutamate.

In some such antibodies, (i) the variable heavy domain comprises a vhCDR1, a vhCDR2, and a vhCDR3 contained within a variable heavy domain comprising the amino acid sequence of SEQ ID NO: 1200, and (ii) the variable light domain comprises a vlCDR1, a vlCDR2, and a vlCDR3 contained within a variable light domain comprising the amino acid sequence of SEQ ID NO: 1204, optionally wherein the CDRs are as defined by the Kabat, Chothia, Kabat+Chothia, AbM, contact, or IMGT definition. In some such antibodies, (i) the variable heavy domain comprises a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, and (ii) the variable light domain comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such antibodies, (i) the variable heavy domain comprises the amino acid sequence SEQ ID NO: 1200, and (ii) the variable light domain comprises the amino acid sequence of SEQ ID NO: 1204, or variants thereof in which an N-terminal glutamine or glutamate is replaced with pyroglutamate.

In some such antibodies, the antibody is a chimeric or humanized antibody. In some such antibodies, the antibody is an antigen-binding fragment of an intact antibody.

In some such antibodies, the antibody comprises a heavy chain constant domain and a light chain constant domain. In some such antibodies, the heavy chain constant domain is of isotype IgG1.

In some such antibodies, the heavy chain constant domain comprise a set of heterodimerization skew variants. In some such antibodies, the heavy chain constant domain each comprise one or more ablation variants. In some such antibodies, the heavy chain constant domain comprises one or more pI variants. In some such antibodies, the heavy chain constant domain comprises amino acid substitutions E233P/L234V/L235A/G236del/S267K, wherein numbering is according to EU numbering. In some such antibodies, the heavy chain constant domain comprises amino acid substitutions M428L/N434S, wherein numbering is according to EU numbering. In some such antibodies, the heavy chain constant domain comprises amino acid substitutions E233P/L234V/L235A/G236del/S267K and M428L/N434S, wherein numbering is according to EU numbering. In some such antibodies, the heavy chain constant domain comprises amino acid substitutions L368D/K370S, wherein numbering is according to EU numbering. In some such antibodies, the heavy chain constant domain comprises amino acid substitutions N208D/Q295E/N384D/Q418E/N421D, wherein numbering is according to EU numbering. In some such antibodies, the heavy chain constant domain comprises amino acid substitutions E233P/L234V/L235A/G236del/S267K, M428L/N434S, L368D/K370S, and N208D/Q295E/N384D/Q418E/N421D, wherein numbering is according to EU numbering. In some such antibodies, the heavy chain constant domain comprises amino acid substitutions E357Q/S364K, wherein numbering is according to EU numbering. In some such antibodies, the heavy chain constant domain comprises amino acid substitutions E233P/L234V/L235A/G236del/S267K, M428L/N434S, and E357Q/S364K, wherein numbering is according to EU numbering. In some such antibodies, the variable heavy domain comprises amino acid substitution Q39E and the variable light domain comprises amino acid substitution Q38K, wherein numbering is according to Kabat numbering. In some such antibodies, the variable heavy domain comprises amino acid substitution Q39K and the variable light domain comprises amino acid substitution Q38E, wherein numbering is according to Kabat numbering. In some such antibodies, the heavy chain constant domain comprises amino acid substitution A141F and the light chain constant domain comprises amino acid substitution F118A, wherein numbering is according to EU numbering. In some such antibodies, the heavy chain constant domain comprises amino acid substitutions K213E/K218D and the light chain constant domain comprises amino acid substitutions D122K/E123K, wherein numbering is according to EU numbering. In some such antibodies, the variable heavy domain comprises amino acid substitution Q39E and the variable light domain comprises amino acid substitution Q38K according to Kabat numbering, and the heavy chain constant domain comprises amino acid substitutions K213E/K218D according to EU numbering and the light chain constant domain comprises amino acid substitutions D122K/E123K according to Kabat numbering. In some such antibodies, the variable heavy domain comprises amino acid substitution Q39K and the variable light domain comprises amino acid substitution Q38E according to Kabat numbering, and the heavy chain constant domain comprises amino acid substitution A141F according to EU numbering and the light chain constant domain comprises amino acid substitution F118A according to Kabat numbering. In some such antibodies, the variable heavy domain comprises amino acid substitution Q39E and the variable light domain comprises amino acid substitution Q38K according to Kabat numbering, and the heavy chain constant domain comprises amino acid substitutions E233P/L234V/L235A/G236del/S267K, M428L/N434S, L368D/K370S, N208D/Q295E/N384D/Q418E/N421D, and K213E/K218D according to EU numbering and the light chain constant domain comprises amino acid substitutions D122K/E123K according to Kabat numbering. In some such antibodies, the variable heavy domain comprises amino acid substitution Q39K and the variable light domain comprises amino acid substitution Q38E according to Kabat numbering, and the heavy chain constant domain comprises amino acid substitutions E233P/L234V/L235A/G236del/S267K, M428L/N434S, E357Q/S364K, and A141F according to EU numbering and the light chain constant domain comprises amino acid substitution F118A according to Kabat numbering. In some such antibodies, the heavy chain constant domain comprises amino acid substitutions E233P/L234V/L235A/G236del/S267K, M428L/N434S, E357Q/S364K, and K213E/K218D according to EU numbering and the light chain constant domain comprises amino acid substitutions D122K/E123K according to Kabat numbering.

In some such antibodies, the antibody comprises a heavy chain comprising the amino acid sequence of SEQ ID NO: 2240 or a variant thereof in which the N-terminal glutamine is replaced with pyroglutamate and/or the C-terminal lysine or the C-terminal lysine and glycine are removed, and a light chain comprising the amino acid sequence of SEQ ID NO: 2242. In some such antibodies, the antibody comprises a heavy chain comprising the amino acid sequence of SEQ ID NO: 2252 or a variant thereof in which the N-terminal glutamine is replaced with pyroglutamate and/or the C-terminal lysine or the C-terminal lysine and glycine are removed, and a light chain comprising the amino acid sequence of SEQ ID NO: 2242. In some such antibodies, the antibody comprises a heavy chain comprising the amino acid sequence of SEQ ID NO: 2258 or a variant thereof in which the N-terminal glutamine is replaced with pyroglutamate and/or the C-terminal lysine or the C-terminal lysine and glycine are removed, and a light chain comprising the amino acid sequence of SEQ ID NO: 2242. In some such antibodies, the antibody comprises a heavy chain comprising the amino acid sequence of SEQ ID NO: 2264 or a variant thereof in which the C-terminal lysine or the C-terminal lysine and glycine are removed, and a light chain comprising the amino acid sequence of SEQ ID NO: 2265 or a variant thereof in which the N-terminal glutamine is replaced with pyroglutamate. In some such antibodies, the antibody comprises a heavy chain comprising the amino acid sequence of SEQ ID NO: 2264 or a variant thereof in which the C-terminal lysine or the C-terminal lysine and glycine are removed, and a light chain comprising the amino acid sequence of SEQ ID NO: 2269 or a variant thereof in which the N-terminal glutamine is replaced with pyroglutamate. In some such antibodies, the antibody comprises a heavy chain at least 90%, at least 95%, at least 96%, at least 97%, at least 98%, or at least 99% identical to the amino acid sequence of SEQ ID NO: 2240, and a light chain at least 90%, at least 95%, at least 96%, at least 97%, at least 98%, or at least 99% identical to the amino acid sequence of SEQ ID NO: 2242. In some such antibodies, the antibody comprises a heavy chain at least 90%, at least 95%, at least 96%, at least 97%, at least 98%, or at least 99% identical to the amino acid sequence of SEQ ID NO: 2252, and a light chain at least 90%, at least 95%, at least 96%, at least 97%, at least 98%, or at least 99% identical to the amino acid sequence of SEQ ID NO: 2242. In some such antibodies, the antibody comprises a heavy chain at least 90%, at least 95%, at least 96%, at least 97%, at least 98%, or at least 99% identical to the amino acid sequence of SEQ ID NO: 2258, and a light chain at least 90%, at least 95%, at least 96%, at least 97%, at least 98%, or at least 99% identical to the amino acid sequence of SEQ ID NO: 2242. In some such antibodies, the antibody comprises a heavy chain at least 90%, at least 95%, at least 96%, at least 97%, at least 98%, or at least 99% identical to the amino acid sequence of SEQ ID NO: 2264, and a light chain at least 90%, at least 95%, at least 96%, at least 97%, at least 98%, or at least 99% identical to the amino acid sequence of SEQ ID NO: 2265. In some such antibodies, the antibody comprises a heavy chain at least 90%, at least 95%, at least 96%, at least 97%, at least 98%, or at least 99% identical to the amino acid sequence of SEQ ID NO: 2264, and a light chain at least 90%, at least 95%, at least 96%, at least 97%, at least 98%, or at least 99% identical to the amino acid sequence of SEQ ID NO: 2269.

In some such antibodies, the antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the antibody specifically binds human TL1A with a K_{D} of less than about 3E-11 M, less than about 2.9E-11 M, less than about 2.8E-11 M, or less than about 2.7E-11 M. In some such antibodies, the antibody inhibits TL1A activity in a TL1A luciferase reporter assay with an IC₅₀ of less than about 4.5 nM.

In some such antibodies, wherein the antibody is a bispecific antibody or multispecific antibody. In some such antibodies, the bispecific antibody or multispecific antibody further comprises an interleukin-23 (IL23) antigen-binding domain. In some such antibodies, the bispecific antibody or multispecific antibody further comprises a means for binding IL23.

In another aspect, provided are pharmaceutical compositions comprising any of the above antibodies and a pharmaceutically acceptable carrier. Some such pharmaceutical compositions further comprise an antibody comprising an IL23 antigen-binding domain. In another aspect, provided are kits comprising any of the above antibodies or pharmaceutical compositions. In another aspect, provided is a vessel or delivery device comprising any of the above antibodies or pharmaceutical compositions. In another aspect, provided is a nucleic acid composition comprising a nucleic acid or a set of nucleic acids encoding any of the above antibodies. In another aspect, provided is an expression vector composition an expression vector or a set of expression vectors comprising the nucleic acid composition. In another aspect, provided is a host cell comprising the expression vector composition. In another aspect, provided is a method of making an anti-TL1A antibody, comprising culturing the host cell under conditions wherein anti-TL1A antibody is expressed, and recovering the anti-TL1A antibody.

In another aspect, provided is a method of inhibiting or reducing TL1A-mediated activity in a subject in need thereof, comprising administering to the subject any of the above antibodies or pharmaceutical compositions. In another aspect, provided are any of the above antibodies or pharmaceutical compositions for use in inhibiting or reducing TL1A-mediated activity in a subject in need thereof. In another aspect, provided is use of any of the above antibodies or pharmaceutical compositions for inhibiting or reducing TL1A-mediated activity in a subject in need thereof. In another aspect, provided is use of any of the above antibodies or pharmaceutical compositions in the manufacture of a medicament for inhibiting or reducing TL1A-mediated activity in a subject in need thereof.

In another aspect, provided is a method of treating a TL1A-associated disease in a subject in need thereof, comprising administering to the subject any of the above antibodies or pharmaceutical compositions. In another aspect, provided are any of the above antibodies or pharmaceutical compositions for use in the treatment of a TL1A-associated disease in a subject in need thereof. In another aspect, provided is use of any of the above antibodies or pharmaceutical compositions for the treatment of a TL1A-associated disease in a subject in need thereof. In another aspect, provided is use of any of the above antibodies or pharmaceutical compositions in the manufacture of a medicament for the treatment of a TL1A-associated disease in a subject in need thereof. In some such methods, antibodies or pharmaceutical compositions for use, or uses, the TL1A-associated disease is an inflammatory disease, an autoimmune disease, or a fibrotic disease, optionally wherein the TL1A-associated disease is an inflammatory bowel disease. In some such methods, antibodies or pharmaceutical compositions for use, or uses, the subject is a human. In some such methods, antibodies or pharmaceutical compositions for use, or uses, the antibody comprising the TL1A-antigen binding domain is used in combination with an antibody comprising an IL23 antigen-binding domain. In some such methods, antibodies or pharmaceutical compositions for use, or uses, the antibody comprising the IL23 antigen-binding domain and the antibody comprising the TL1A antigen-binding domain are administered simultaneously. In some such methods, antibodies or pharmaceutical compositions for use, or uses, the antibody comprising the IL23 antigen-binding domain and the antibody comprising the TL1A antigen-binding domain are administered sequentially in any order.

In another aspect, provided are antibodies comprising an interleukin-23 (IL23) antigen-binding domain comprising a variable heavy domain and a variable light domain. In some such antibodies, the antibody comprises a variable heavy domain comprising a vhCDR1, a vhCDR2, and a vhCDR3 contained within a variable heavy domain comprising the amino acid sequence of any one of SEQ ID NOS: 2233, 2235, and 179, and a variable light domain comprising a vlCDR1, a vlCDR2, and a vlCDR3 contained within a variable light domain comprising the amino acid sequence of any one of SEQ ID NOS: 2234, 2237, and 1248. In some such antibodies, the antibody comprises a variable heavy domain comprising a vhCDR1, a vhCDR2, and a vhCDR3 contained within a variable heavy domain comprising the amino acid sequence of SEQ ID NO: 2233, and a variable light domain comprising a vlCDR1, a vlCDR2, and a vlCDR3 contained within a variable light domain comprising the amino acid sequence of SEQ ID NO: 2234. In some such antibodies, the antibody comprises a variable heavy domain comprising a vhCDR1, a vhCDR2, and a vhCDR3 contained within a variable heavy domain comprising the amino acid sequence of SEQ ID NO: 2235, and a variable light domain comprising a vlCDR1, a vlCDR2, and a vlCDR3 contained within a variable light domain comprising the amino acid sequence of SEQ ID NO: 2237. In some such antibodies, the antibody comprises a variable heavy domain comprising a vhCDR1, a vhCDR2, and a vhCDR3 contained within a variable heavy domain comprising the amino acid sequence of SEQ ID NO: 179, and a variable light domain comprising a vlCDR1, a vlCDR2, and a vlCDR3 contained within a variable light domain comprising the amino acid sequence of SEQ ID NO: 1248. In some such antibodies, the antibody comprises a variable heavy domain comprising a vhCDR1, a vhCDR2, and a vhCDR3 contained within a variable heavy domain comprising the amino acid sequence of SEQ ID NO: 2113, and a variable light domain comprising a vlCDR1, a vlCDR2, and a vlCDR3 contained within a variable light domain comprising the amino acid sequence of SEQ ID NO: 2231. In some such antibodies, the antibody comprises a variable heavy domain comprising a vhCDR1, a vhCDR2, and a vhCDR3 contained within a variable heavy domain comprising the amino acid sequence of SEQ ID NO: 1967, and a variable light domain comprising a vlCDR1, a vlCDR2, and a vlCDR3 contained within a variable light domain comprising the amino acid sequence of SEQ ID NO: 1248. In some such antibodies, the antibody comprises a variable heavy domain comprising a vhCDR1, a vhCDR2, and a vhCDR3 and a variable light domain comprising a vlCDR1, a vlCDR2, and a vlCDR3 contained within a variable heavy domain and variable light domain pair in XENP53412, XENP53413, XENP53369, XENP53375, XENP53387, XENP53415, XENP53370, XENP53371, XENP53376, XENP53377, XENP53378, XENP53379, XENP53380, or XENP53414. Optionally, the CDRs in any of the above antibodies are as defined by the Kabat, Chothia, Kabat+Chothia, AbM, contact, or IMGT definition.

In some such antibodies, the variable heavy domain comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182 or 2236, and the variable light domain comprises a vlCDR1 comprising the amino acid sequence of SEQ ID NO: 184, a vlCDR2 comprising the amino acid sequence of SEQ ID NO: 1249 or 2238, and a vlCDR3 comprising the amino acid sequence of SEQ ID NO: 1250. In some such antibodies, the variable heavy domain comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and the variable light domain comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, the variable heavy domain comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 2236, and the variable light domain comprises a vlCDR1 comprising the amino acid sequence of SEQ ID NO: 184, a vlCDR2 comprising the amino acid sequence of SEQ ID NO: 2238, and a vlCDR3 comprising the amino acid sequence of SEQ ID NO: 1250. In some such antibodies, the variable heavy domain comprises a vhCDR1 comprising the amino acid sequence of SEQ ID NO: 2239, a vhCDR2 comprising the amino acid sequence of SEQ ID NO: 181, and a vhCDR3 comprising the amino acid sequence of SEQ ID NO: 182, and the variable light domain comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250.

In some such antibodies, the variable heavy domain is at least 90%, at least 95%, at least 96%, at least 97%, at least 98%, or at least 99% identical to the amino acid sequence of any one of SEQ ID NOS: 2233, 2235, and 179, and the variable light domain is at least 90%, at least 95%, at least 96%, at least 97%, at least 98%, or at least 99% identical to the amino acid sequence of any one of SEQ ID NOS: 2234, 2237, and 1248. In some such antibodies, the variable heavy domain is at least 90%, at least 95%, at least 96%, at least 97%, at least 98%, or at least 99% identical to the amino acid sequence of SEQ ID NO: 2233, and the variable light domain is at least 90%, at least 95%, at least 96%, at least 97%, at least 98%, or at least 99% identical to the amino acid sequence of SEQ ID NO: 2234. In some such antibodies, the variable heavy domain is at least 90%, at least 95%, at least 96%, at least 97%, at least 98%, or at least 99% identical to the amino acid sequence of SEQ ID NO: 2235, and the variable light domain is at least 90%, at least 95%, at least 96%, at least 97%, at least 98%, or at least 99% identical to the amino acid sequence of SEQ ID NO: 2237. In some such antibodies, the variable heavy domain is at least 90%, at least 95%, at least 96%, at least 97%, at least 98%, or at least 99% identical to the amino acid sequence of SEQ ID NO: 179, and the variable light domain is at least 90%, at least 95%, at least 96%, at least 97%, at least 98%, or at least 99% identical to the amino acid sequence of SEQ ID NO: 1248. In some such antibodies, the variable heavy domain is at least 90%, at least 95%, at least 96%, at least 97%, at least 98%, or at least 99% identical to the amino acid sequence of SEQ ID NO: 2113, and the variable light domain is at least 90%, at least 95%, at least 96%, at least 97%, at least 98%, or at least 99% identical to the amino acid sequence of SEQ ID NO: 2231. In some such antibodies, the variable heavy domain is at least 90%, at least 95%, at least 96%, at least 97%, at least 98%, or at least 99% identical to the amino acid sequence of SEQ ID NO: 1967, and the variable light domain is at least 90%, at least 95%, at least 96%, at least 97%, at least 98%, or at least 99% identical to the amino acid sequence of SEQ ID NO: 1248. In some such antibodies, the variable heavy domain is at least 90%, at least 95%, at least 96%, at least 97%, at least 98%, or at least 99% identical to the amino acid sequence of any one of SEQ ID NOS: 1927-2132, and the variable light domain is at least 90%, at least 95%, at least 96%, at least 97%, at least 98%, or at least 99% identical to the amino acid sequence of any one of SEQ ID NOS: 2133-2232. In some such antibodies, the variable heavy domain and the variable light domain are at least 90%, at least 95%, at least 96%, at least 97%, at least 98%, or at least 99% identical to any pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the variable heavy domain and the variable light domain are at least 90%, at least 95%, at least 96%, at least 97%, at least 98%, or at least 99% identical to any pair of anti-IL23 variable heavy domain and variable light domain in Table 29 or 33.

In some such antibodies, the variable heavy domain comprises the amino acid sequence of any one of SEQ ID NOS: 2233, 2235, and 179, and the variable light domain comprises the amino acid sequence of any one of SEQ ID NOS: 2234, 2237, and 1248. In some such antibodies, the variable heavy domain comprises the amino acid sequence of SEQ ID NO: 2233, and the variable light domain comprises the amino acid sequence of SEQ ID NO: 2234. In some such antibodies, the variable heavy domain comprises the amino acid sequence of SEQ ID NO: 2235, and the variable light domain comprises the amino acid sequence of SEQ ID NO: 2237. In some such antibodies, the variable heavy domain comprises the amino acid sequence of SEQ ID NO: 179, and the variable light domain comprises the amino acid sequence of SEQ ID NO: 1248. In some such antibodies, the variable heavy domain comprises the amino acid sequence of SEQ ID NO: 2113, and the variable light domain comprises the amino acid sequence of SEQ ID NO: 2231. In some such antibodies, the variable heavy domain comprises the amino acid sequence of SEQ ID NO: 1967, and the variable light domain comprises the amino acid sequence of SEQ ID NO: 1248. In some such antibodies, the variable heavy domain comprises the amino acid sequence of any one of SEQ ID NOS: 1927-2132, and the variable light domain comprises the amino acid sequence of any one of SEQ ID NOS: 2133-2232. In some such antibodies, the variable heavy domain and the variable light domain comprise any pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such antibodies, the variable heavy domain comprises the amino acid sequence of any one of SEQ ID NOS: 1927-2132, and the variable light domain comprises the amino acid sequence of any one of SEQ ID NOS: 2133-2232. In some such antibodies, the variable heavy domain and the variable light domain comprise any pair of anti-IL23 variable heavy domain and variable light domain in Table 29 or 33. In some such antibodies, the variable heavy domain or the variable light domain can comprise variants of any of the above in which an N-terminal glutamine or glutamate is replaced with pyroglutamate.

In some such antibodies, (i) the variable heavy domain comprises a vhCDR1, a vhCDR2, and a vhCDR3 contained within a variable heavy domain comprising the amino acid sequence of any one of SEQ ID NOS: 2233, 2235, and 179, and (ii) the variable light domain comprises a vlCDR1, a vlCDR2, and a vlCDR3 contained within a variable light domain comprising the amino acid sequence of any one of SEQ ID NOS: 2234, 2237, and 1248, optionally wherein the CDRs are as defined by the Kabat, Chothia, Kabat+Chothia, AbM, contact, or IMGT definition. In some such antibodies, (i) the variable heavy domain comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182 or 2236, and (ii) the variable light domain comprises a vlCDR1 comprising the amino acid sequence of SEQ ID NO: 184, a vlCDR2 comprising the amino acid sequence of SEQ ID NO: 1249 or 2238, and a vlCDR3 comprising the amino acid sequence of SEQ ID NO: 1250. In some such antibodies, (i) the variable heavy domain comprises the amino acid sequence of any one of SEQ ID NOS: 2233, 2235, and 179, and (ii) the variable light domain comprises the amino acid sequence of any one of SEQ ID NOS: 2234, 2237, and 1248, or variants thereof in which an N-terminal glutamine or glutamate is replaced with pyroglutamate. In some such antibodies, the antibody specifically binds human TL23 with a K_{D} of less than about 1.1E-10 M as measured by surface plasmon resonance or Biacore at 25° C., optionally wherein the antibody specifically binds human TL23 with a K_{D} of less than about 6E-11 M, less than about 5.9E-11 M, less than about 5.8E-11 M, or less than about 5.7E-11 M. In some such antibodies, the antibody inhibits TL23 activity in an IL23 luciferase reporter assay with an IC₅₀ of less than about 65 pM, less than about 64 pM, or less than about 63 pM, optionally wherein the antibody inhibits IL23 activity with an IC₅₀ of less than about 60 pM, less than about 59 pM, less than about 58 pM, less than about 57 pM, or less than about 56 pM

In some such antibodies, (i) the variable heavy domain comprises a vhCDR1, a vhCDR2, and a vhCDR3 contained within a variable heavy domain comprising the amino acid sequence of SEQ ID NO: 2233, and (ii) the variable light domain comprises a vlCDR1, a vlCDR2, and a vlCDR3 contained within a variable light domain comprising the amino acid sequence of SEQ ID NO: 2234, optionally wherein the CDRs are as defined by the Kabat, Chothia, Kabat+Chothia, AbM, contact, or IMGT definition. In some such antibodies, (i) the variable heavy domain comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (ii) the variable light domain comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, (i) the variable heavy domain comprises the amino acid sequence of SEQ ID NO: 2233, and (ii) the variable light domain comprises the amino acid sequence of SEQ ID NO: 2234, or variants thereof in which an N-terminal glutamine or glutamate is replaced with pyroglutamate. In some such antibodies, the antibody specifically binds human TL23 with a K_{D} of less than about 1.1E-10 M as measured by surface plasmon resonance or Biacore at 25° C., optionally wherein the antibody specifically binds human IL23 with a K_{D} of less than about 6E-11 M, less than about 5.9E-11 M, less than about 5.8E-11 M, or less than about 5.7E-11 M. In some such antibodies, the antibody inhibits TL23 activity in an IL23 luciferase reporter assay with an IC₅₀ of less than about 65 pM, less than about 64 pM, or less than about 63 pM, optionally wherein the antibody inhibits IL23 activity with an IC₅₀ of less than about 60 pM, less than about 59 pM, less than about 58 pM, less than about 57 pM, or less than about 56 pM

In some such antibodies, (i) the variable heavy domain comprises a vhCDR1, a vhCDR2, and a vhCDR3 contained within a variable heavy domain comprising the amino acid sequence of SEQ ID NO: 2235, and (ii) the variable light domain comprises a vlCDR1, a vlCDR2, and a vlCDR3 contained within a variable light domain comprising the amino acid sequence of SEQ ID NO: 2237, optionally wherein the CDRs are as defined by the Kabat, Chothia, Kabat+Chothia, AbM, contact, or IMGT definition. In some such antibodies, (i) the variable heavy domain comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 2236, and (ii) the variable light domain comprises a vlCDR1 comprising the amino acid sequence of SEQ ID NO: 184, a vlCDR2 comprising the amino acid sequence of SEQ ID NO: 2238, and a vlCDR3 comprising the amino acid sequence of SEQ ID NO: 1250. In some such antibodies, (i) the variable heavy domain comprises the amino acid sequence of SEQ ID NO: 2235, and (ii) the variable light domain comprises the amino acid sequence of SEQ ID NO: 2237, or variants thereof in which an N-terminal glutamine or glutamate is replaced with pyroglutamate.

In some such antibodies, (i) the variable heavy domain comprises a vhCDR1, a vhCDR2, and a vhCDR3 contained within a variable heavy domain comprising the amino acid sequence of SEQ ID NO: 179, and (ii) the variable light domain comprises a vlCDR1, a vlCDR2, and a vlCDR3 contained within a variable light domain comprising the amino acid sequence of SEQ ID NO: 1248, optionally wherein the CDRs are as defined by the Kabat, Chothia, Kabat+Chothia, AbM, contact, or IMGT definition. In some such antibodies, (i) the variable heavy domain comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (ii) the variable light domain comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such antibodies, (i) the variable heavy domain comprises the amino acid sequence of SEQ ID NO: 179, and (ii) the variable light domain comprises the amino acid sequence of SEQ ID NO: 1248, or variants thereof in which an N-terminal glutamine or glutamate is replaced with pyroglutamate.

In some such antibodies, the antibody is a chimeric or humanized antibody. In some such antibodies, the antibody is an antigen-binding fragment of an intact antibody. In some such antibodies, the antibody comprises a heavy chain constant domain and a light chain constant domain. In some such antibodies, the heavy chain constant domain is of isotype IgG1.

In some such antibodies, the heavy chain constant domain comprise a set of heterodimerization skew variants. In some such antibodies, the heavy chain constant domain each comprise one or more ablation variants. In some such antibodies, the heavy chain constant domain comprises one or more pI variants. In some such antibodies, the heavy chain constant domain comprises amino acid substitutions E233P/L234V/L235A/G236del/S267K, wherein numbering is according to EU numbering. In some such antibodies, the heavy chain constant domain comprises amino acid substitutions M428L/N434S, wherein numbering is according to EU numbering. In some such antibodies, the heavy chain constant domain comprises amino acid substitutions E233P/L234V/L235A/G236del/S267K and M428L/N434S, wherein numbering is according to EU numbering. In some such antibodies, the heavy chain constant domain comprises amino acid substitutions L368D/K370S, wherein numbering is according to EU numbering. In some such antibodies, the heavy chain constant domain comprises amino acid substitutions N208D/Q295E/N384D/Q418E/N421D, wherein numbering is according to EU numbering. In some such antibodies, the heavy chain constant domain comprises amino acid substitutions E233P/L234V/L235A/G236del/S267K, M428L/N434S, L368D/K370S, and N208D/Q295E/N384D/Q418E/N421D, wherein numbering is according to EU numbering. In some such antibodies, the heavy chain constant domain comprises amino acid substitutions E357Q/S364K, wherein numbering is according to EU numbering. In some such antibodies, the heavy chain constant domain comprises amino acid substitutions E233P/L234V/L235A/G236del/S267K, M428L/N434S, and E357Q/S364K, wherein numbering is according to EU numbering. In some such antibodies, the variable heavy domain comprises amino acid substitution Q39E and the variable light domain comprises amino acid substitution Q38K, wherein numbering is according to Kabat numbering. In some such antibodies, the variable heavy domain comprises amino acid substitution Q39K and the variable light domain comprises amino acid substitution Q38E, wherein numbering is according to Kabat numbering. In some such antibodies, the heavy chain constant domain comprises amino acid substitution A141F and the light chain constant domain comprises amino acid substitution F118A, wherein numbering is according to EU numbering. In some such antibodies, the heavy chain constant domain comprises amino acid substitutions K213E/K218D and the light chain constant domain comprises amino acid substitutions D122K/E123K, wherein numbering is according to EU numbering. In some such antibodies, the variable heavy domain comprises amino acid substitution Q39E and the variable light domain comprises amino acid substitution Q38K according to Kabat numbering, and the heavy chain constant domain comprises amino acid substitutions K213E/K218D according to EU numbering and the light chain constant domain comprises amino acid substitutions D122K/E123K according to Kabat numbering. In some such antibodies, the variable heavy domain comprises amino acid substitution Q39K and the variable light domain comprises amino acid substitution Q38E according to Kabat numbering, and the heavy chain constant domain comprises amino acid substitution A141F according to EU numbering and the light chain constant domain comprises amino acid substitution F118A according to Kabat numbering. In some such antibodies, the variable heavy domain comprises amino acid substitution Q39E and the variable light domain comprises amino acid substitution Q38K according to Kabat numbering, and the heavy chain constant domain comprises amino acid substitutions E233P/L234V/L235A/G236del/S267K, M428L/N434S, L368D/K370S, N208D/Q295E/N384D/Q418E/N421D, and K213E/K218D according to EU numbering and the light chain constant domain comprises amino acid substitutions D122K/E123K according to Kabat numbering. In some such antibodies, the variable heavy domain comprises amino acid substitution Q39K and the variable light domain comprises amino acid substitution Q38E according to Kabat numbering, and the heavy chain constant domain comprises amino acid substitutions E233P/L234V/L235A/G236del/S267K, M428L/N434S, E357Q/S364K, and A141F according to EU numbering and the light chain constant domain comprises amino acid substitution F118A according to Kabat numbering. In some such antibodies, the heavy chain constant domain comprises amino acid substitutions E233P/L234V/L235A/G236del/S267K, M428L/N434S, E357Q/S364K, and K213E/K218D according to EU numbering and the light chain constant domain comprises amino acid substitutions D122K/E123K according to Kabat numbering.

In some such antibodies, the antibody comprises a heavy chain comprising the amino acid sequence of SEQ ID NO: 2244 or a variant thereof in which the N-terminal glutamine is replaced with pyroglutamate and/or the C-terminal lysine or the C-terminal lysine and glycine are removed, and a light chain comprising the amino acid sequence of SEQ ID NO: 2245. In some such antibodies, the antibody comprises a heavy chain comprising the amino acid sequence of SEQ ID NO: 2250 or a variant thereof in which the N-terminal glutamine is replaced with pyroglutamate and/or the C-terminal lysine or the C-terminal lysine and glycine are removed, and a light chain comprising the amino acid sequence of SEQ ID NO: 2251. In some such antibodies, the antibody comprises a heavy chain comprising the amino acid sequence of SEQ ID NO: 2266 or a variant thereof in which the N-terminal glutamine is replaced with pyroglutamate and/or the C-terminal lysine or the C-terminal lysine and glycine are removed, and a light chain comprising the amino acid sequence of SEQ ID NO: 2267. In some such antibodies, the antibody comprises a heavy chain at least 90%, at least 95%, at least 96%, at least 97%, at least 98%, or at least 99% identical to the amino acid sequence of SEQ ID NO: 2244, and a light chain at least 90%, at least 95%, at least 96%, at least 97%, at least 98%, or at least 99% identical to the amino acid sequence of SEQ ID NO: 2245. In some such antibodies, the antibody comprises a heavy chain at least 90%, at least 95%, at least 96%, at least 97%, at least 98%, or at least 99% identical to the amino acid sequence of SEQ ID NO: 2250, and a light chain at least 90%, at least 95%, at least 96%, at least 97%, at least 98%, or at least 99% identical to the amino acid sequence of SEQ ID NO: 2251. In some such antibodies, the antibody comprises a heavy chain is at least 90%, at least 95%, at least 96%, at least 97%, at least 98%, or at least 99% identical to the amino acid sequence of SEQ ID NO: 2266, and a light chain is at least 90%, at least 95%, at least 96%, at least 97%, at least 98%, or at least 99% identical to the amino acid sequence of SEQ ID NO: 2267.

In some such antibodies, the antibody specifically binds human IL23 with a K_{D} of less than about 1.1E-10 M as measured by surface plasmon resonance or Biacore at 25° C., optionally wherein the antibody specifically binds human IL23 with a K_{D} of less than about 6E-11 M, less than about 5.9E-11 M, less than about 5.8E-11 M, or less than about 5.7E-11 M. In some such antibodies, the antibody inhibits IL23 activity in an IL23 luciferase reporter assay with an IC₅₀ of less than about 65 pM, less than about 64 pM, or less than about 63 pM, optionally wherein the antibody inhibits TL23 activity with an IC₅₀ of less than about 60 pM, less than about 59 pM, less than about 58 pM, less than about 57 pM, or less than about 56 pM.

In some such antibodies, the antibody is a bispecific antibody or multispecific antibody. In some such antibodies, the bispecific antibody or multispecific antibody further comprises a TNF-like ligand 1A (TL1A) antigen-binding domain. In some such antibodies, the bispecific antibody or multispecific antibody further comprises a means for binding TL1A.

In another aspect, provided are pharmaceutical compositions comprising any of the above antibodies and a pharmaceutically acceptable carrier. Some such pharmaceutical compositions further comprise an antibody comprising an TL1A antigen-binding domain.

In another aspect, provided is a kit comprising any of the above antibodies or pharmaceutical compositions. In another aspect, provided is a vessel or delivery device comprising any of the above antibodies or pharmaceutical compositions. In another aspect, provided is a nucleic acid composition comprising a nucleic acid or a set of nucleic acids encoding any of the above antibodies. In another aspect, provided is an expression vector composition an expression vector or a set of expression vectors comprising the nucleic acid composition. In another aspect, provided is a host cell comprising the expression vector composition. In another aspect, provided is a method of making an anti-IL23 antibody, comprising culturing the host cell under conditions wherein anti-IL23 antibody is expressed, and recovering the anti-IL23 antibody.

In another aspect, provided is a method of inhibiting or reducing IL23-mediated activity in a subject in need thereof, comprising administering to the subject any of the above antibodies or pharmaceutical compositions. In another aspect, provided are of the above antibodies or pharmaceutical compositions for use in inhibiting or reducing TL23-mediated activity in a subject in need thereof. In another aspect, provided is use of any of the above antibodies or pharmaceutical compositions for inhibiting or reducing IL23-mediated activity in a subject in need thereof. In another aspect, provided is use of any of the above antibodies or pharmaceutical compositions in the manufacture of a medicament for inhibiting or reducing IL23-mediated activity in a subject in need thereof.

In another aspect, provided is a method of treating an IL23-associated disease in a subject in need thereof, comprising administering to the subject any of the above antibodies or pharmaceutical composition. In another aspect, provided are any of the above antibodies or pharmaceutical compositions for use in the treatment of an IL23-associated disease in a subject in need thereof. In another aspect, provided is use of any of the above antibodies or pharmaceutical compositions for the treatment of an IL23-associated disease in a subject in need thereof. In another aspect, provided is use of any of the above antibodies or pharmaceutical compositions in the manufacture of a medicament for the treatment of an IL23-associated disease in a subject in need thereof. In some such methods, antibodies or compositions for use, or uses, the IL23-associated disease is an inflammatory disease, an autoimmune disease, or a fibrotic disease, optionally wherein the IL23-associated disease is an inflammatory bowel disease. In some such methods, antibodies or compositions for use, or uses, the subject is a human. In some such methods, antibodies or compositions for use, or uses, the antibody comprising the IL23-antigen binding domain is used in combination with an antibody comprising an TL1A antigen-binding domain. In some such methods, antibodies or compositions for use, or uses, the antibody comprising the TL1A antigen-binding domain and the antibody comprising the IL23 antigen-binding domain are administered simultaneously. In some such methods, antibodies or compositions for use, or uses, the antibody comprising the TL1A antigen-binding domain and the antibody comprising the IL23 antigen-binding domain are administered sequentially in any order.

In another aspect, provided are combinations comprising: (a) a first antibody comprising a TNF-like ligand 1A (TL1A) antigen-binding domain comprising a variable heavy domain (VH1) and a variable light domain (VL1); and (b) a second antibody comprising an interleukin-23 (IL23) antigen-binding domain comprising a variable heavy domain (VH2) and a variable light domain (VL2).

In some such combinations, the VH1 and VL1 comprise a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in Table 44, optionally wherein the CDRs are as defined by the Kabat, Chothia, Kabat+Chothia, AbM, contact, or IMGT definition. In some such combinations, the VH1 and VL1 comprise a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in Tables 7-10, 22, 23, 26, 29-34, and 48, optionally wherein the CDRs are as defined by the Kabat, Chothia, Kabat+Chothia, AbM, contact, or IMGT definition. In some such combinations, the VH1 and VL1 comprise a variable heavy domain comprising a vhCDR1, a vhCDR2, and a vhCDR3 contained within a variable heavy domain comprising the amino acid sequence of any one of SEQ ID NOS: 1427-1823 and 2316-2330, and a variable light domain comprising a vlCDR1, a vlCDR2, and a vlCDR3 contained within a variable light domain comprising the amino acid sequence of any one of 1824-1923, optionally wherein the CDRs are as defined by the Kabat, Chothia, Kabat+Chothia, AbM, contact, or IMGT definition. In some such combinations, the VH1 and VL1 comprise any pair of anti-TL1A variable heavy domain and variable light domain in Table 44. In some such combinations, the VH1 and VL1 comprise any pair of anti-TL1A variable heavy domain and variable light domain in Tables 7-10, 22, 23, 26, 29-34, and 48. In some such combinations, the VH1 and VL1 comprise a variable heavy domain comprising the amino acid sequence of any one of SEQ ID NOS: 1427-1823 and 2316-2330, and a variable light domain comprising the amino acid sequence of any one of 1824-1923, or variants thereof in which an N-terminal glutamine or glutamate is replaced with pyroglutamate. In some such combinations, the VH2 and VL2 comprise a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in Table 44, optionally wherein the CDRs are as defined by the Kabat, Chothia, Kabat+Chothia, AbM, contact, or IMGT definition. In some such combinations, the VH2 and VL2 comprise a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in Tables 11, 24-26, 29-34, and 49, optionally wherein the CDRs are as defined by the Kabat, Chothia, Kabat+Chothia, AbM, contact, or IMGT definition. In some such combinations, the VH2 and VL2 comprise a variable heavy domain comprising a vhCDR1, a vhCDR2, and a vhCDR3 contained within a variable heavy domain comprising the amino acid sequence of any one of 1927-2132, and a variable light domain comprising a vlCDR1, a vlCDR2, and a vlCDR3 contained within a variable light domain comprising the amino acid sequence of any one of 2133-2232, optionally wherein the CDRs are as defined by the Kabat, Chothia, Kabat+Chothia, AbM, contact, or IMGT definition. In some such combinations, the VH2 and VL2 comprise any pair of anti-IL23 variable heavy domain and variable light domain in Table 44. In some such combinations, the VH2 and VL2 comprise any pair of anti-IL23 variable heavy domain and variable light domain in Tables 11, 24-26, 29-34, and 49. In some such combinations, the VH2 and VL2 comprise a variable heavy domain comprising the amino acid sequence of any one of 1927-2132, and a variable light domain comprising the amino acid sequence of any one of 2133-2232, or variants thereof in which an N-terminal glutamine or glutamate is replaced with pyroglutamate.

In some such combinations, the VH1 and VL1 comprise a variable heavy domain comprising a vhCDR1, a vhCDR2, and a vhCDR3 contained within a variable heavy domain comprising the amino acid sequence of any one of SEQ ID NOS: 2241, 2253, 2259, 1674, and 1200, and a variable light domain comprising a vlCDR1, a vlCDR2, and a vlCDR3 contained within a variable light domain comprising the amino acid sequence of any one of SEQ ID NOS: 2243 and 1204. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 2241, and a variable light domain comprising a vlCDR1, a vlCDR2, and a vlCDR3 contained within a variable light domain comprising the amino acid sequence of SEQ ID NO: 2243. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 2253, and a variable light domain comprising a vlCDR1, a vlCDR2, and a vlCDR3 contained within a variable light domain comprising the amino acid sequence of SEQ ID NO: 2243. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 2259, and a variable light domain comprising a vlCDR1, a vlCDR2, and a vlCDR3 contained within a variable light domain comprising the amino acid sequence of SEQ ID NO: 2243. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 1674, and a variable light domain comprising a vlCDR1, a vlCDR2, and a vlCDR3 contained within a variable light domain comprising the amino acid sequence of SEQ ID NO: 1204. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 1200, and a variable light domain comprising a vlCDR1, a vlCDR2, and a vlCDR3 contained within a variable light domain comprising the amino acid sequence of SEQ ID NO: 1204. In some such combinations, the VH1 and VL1 comprise a variable heavy domain comprising a vhCDR1, a vhCDR2, and a vhCDR3 contained within a variable heavy domain comprising the amino acid sequence of any one of SEQ ID NOS: 1427-1823 and 2316-2330, and a variable light domain comprising a vlCDR1, a vlCDR2, and a vlCDR3 contained within a variable light domain comprising the amino acid sequence of any one of SEQ ID NOS: 1824-1923. In some such combinations, the VH1 and VL1 comprise a variable heavy domain comprising a vhCDR1, a vhCDR2, and a vhCDR3 contained within a variable heavy domain comprising the amino acid sequence of any one of SEQ ID NOS: 1674, 1200, 1661, and 2323, and a variable light domain comprising a vlCDR1, a vlCDR2, and a vlCDR3 contained within a variable light domain comprising the amino acid sequence of SEQ ID NO: 1204. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 1661, and a variable light domain comprising a vlCDR1, a vlCDR2, and a vlCDR3 contained within a variable light domain comprising the amino acid sequence of SEQ ID NO: 1204. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 2323, and a variable light domain comprising a vlCDR1, a vlCDR2, and a vlCDR3 contained within a variable light domain comprising the amino acid sequence of SEQ ID NO: 1204. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 101, and a variable light domain comprising a vlCDR1, a vlCDR2, and a vlCDR3 contained within a variable light domain comprising the amino acid sequence of SEQ ID NO: 105. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 109, and a variable light domain comprising a vlCDR1, a vlCDR2, and a vlCDR3 contained within a variable light domain comprising the amino acid sequence of SEQ ID NO: 113. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 117, and a variable light domain comprising a vlCDR1, a vlCDR2, and a vlCDR3 contained within a variable light domain comprising the amino acid sequence of SEQ ID NO: 121. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 125, and a variable light domain comprising a vlCDR1, a vlCDR2, and a vlCDR3 contained within a variable light domain comprising the amino acid sequence of SEQ ID NO: 129. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 133, and a variable light domain comprising a vlCDR1, a vlCDR2, and a vlCDR3 contained within a variable light domain comprising the amino acid sequence of SEQ ID NO: 137. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 141, and a variable light domain comprising a vlCDR1, a vlCDR2, and a vlCDR3 contained within a variable light domain comprising the amino acid sequence of SEQ ID NO: 145. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 149, and a variable light domain comprising a vlCDR1, a vlCDR2, and a vlCDR3 contained within a variable light domain comprising the amino acid sequence of SEQ ID NO: 153. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 157, and a variable light domain comprising a vlCDR1, a vlCDR2, and a vlCDR3 contained within a variable light domain comprising the amino acid sequence of SEQ ID NO: 159. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 161, and a variable light domain comprising a vlCDR1, a vlCDR2, and a vlCDR3 contained within a variable light domain comprising the amino acid sequence of SEQ ID NO: 165. In some such combinations, the VH1 and VL1 comprise a variable heavy domain comprising a vhCDR1, a vhCDR2, and a vhCDR3 contained within a variable heavy domain comprising the amino acid sequence of SEQ ID NO: 169, and a variable light domain comprising a vlCDR1, a vlCDR2, and a vlCDR3 contained within a variable light domain comprising the amino acid sequence of SEQ ID NO: 173. In some such combinations, the VH1 and VL1 comprise a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such combinations, the VH1 and VL1 comprise a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in Tables 29 and 31-34. Optionally, the CDRs in any of the above antibodies are as defined by the Kabat, Chothia, Kabat+Chothia, AbM, contact, or IMGT definition.

In some such combinations, the VH1 and VL1 comprise a variable heavy domain comprising a vhCDR1 comprising the amino acid sequence of SEQ ID NO: 102 or 1201, a vhCDR2 comprising the amino acid sequence of SEQ ID NO: 1924 or 1202, and a vhCDR3 comprising the amino acid sequence of SEQ ID NO: 1203 or 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such combinations, the VH1 and VL1 comprise a variable heavy domain comprising a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such combinations, the VH1 and VL1 comprise a variable heavy domain comprising a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such combinations, the VH1 and VL1 comprise a variable heavy domain comprising a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such combinations, the VH1 and VL1 comprise a variable heavy domain comprising a vhCDR1 comprising the amino acid sequence of SEQ ID NO: 102, a vhCDR2 comprising the amino acid sequence of SEQ ID NO: 1926, and a vhCDR3 comprising the amino acid sequence of SEQ ID NO: 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such combinations, the VH1 and VL1 comprise a variable heavy domain comprising a vhCDR1 comprising the amino acid sequence of SEQ ID NO: 102, a vhCDR2 comprising the amino acid sequence of SEQ ID NO: 103, and a vhCDR3 comprising the amino acid sequence of SEQ ID NO: 104, and a variable light domain comprising a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some such combinations, the VH1 and VL1 comprise a variable heavy domain comprising a vhCDR1 comprising the amino acid sequence of SEQ ID NO: 110, a vhCDR2 comprising the amino acid sequence of SEQ ID NO: 111, and a vhCDR3 comprising the amino acid sequence of SEQ ID NO: 112, and a variable light domain comprising a vlCDR1 comprising the amino acid sequence of SEQ ID NO: 114, a vlCDR2 comprising the amino acid sequence of SEQ ID NO: 115, and a vlCDR3 comprising the amino acid sequence of SEQ ID NO: 116. In some such combinations, the VH1 and VL1 comprise a variable heavy domain comprising a vhCDR1 comprising the amino acid sequence of SEQ ID NO: 118, a vhCDR2 comprising the amino acid sequence of SEQ ID NO: 119, and a vhCDR3 comprising the amino acid sequence of SEQ ID NO: 120, and a variable light domain comprising a vlCDR1 comprising the amino acid sequence of SEQ ID NO: 122, a vlCDR2 comprising the amino acid sequence of SEQ ID NO: 123, and a vlCDR3 comprising the amino acid sequence of SEQ ID NO: 124. In some such combinations, the VH1 and VL1 comprise a variable heavy domain comprising a vhCDR1 comprising the amino acid sequence of SEQ ID NO: 126, a vhCDR2 comprising the amino acid sequence of SEQ ID NO: 127, and a vhCDR3 comprising the amino acid sequence of SEQ ID NO: 128, and a variable light domain comprising a vlCDR1 comprising the amino acid sequence of SEQ ID NO: 130, a vlCDR2 comprising the amino acid sequence of SEQ ID NO: 131, and a vlCDR3 comprising the amino acid sequence of the amino acid sequence of SEQ ID NO: 132. In some such combinations, the VH1 and VL1 comprise a variable heavy domain comprising a vhCDR1 comprising the amino acid sequence of SEQ ID NO: 134, a vhCDR2 comprising the amino acid sequence of SEQ ID NO: 135, and a vhCDR3 comprising the amino acid sequence of SEQ ID NO: 136, and a variable light domain comprising a vlCDR1 comprising the amino acid sequence of SEQ ID NO: 138, a vlCDR2 comprising the amino acid sequence of SEQ ID NO: 139, and a vlCDR3 comprising the amino acid sequence of the amino acid sequence of SEQ ID NO: 140. In some such combinations, the VH1 and VL1 comprise a variable heavy domain comprising a vhCDR1 comprising the amino acid sequence of SEQ ID NO: 142, a vhCDR2 comprising the amino acid sequence of SEQ ID NO: 143, and a vhCDR3 comprising the amino acid sequence of SEQ ID NO: 144, and a variable light domain comprising a vlCDR1 comprising the amino acid sequence of SEQ ID NO: 146, a vlCDR2 comprising the amino acid sequence of SEQ ID NO: 147, and a vlCDR3 comprising the amino acid sequence of SEQ ID NO: 148. In some such combinations, the VH1 and VL1 comprise a variable heavy domain comprising a vhCDR1 comprising the amino acid sequence of SEQ ID NO: 150, a vhCDR2 comprising the amino acid sequence of SEQ ID NO: 151, and a vhCDR3 comprising the amino acid sequence of SEQ ID NO: 152, and a variable light domain comprising a vlCDR1 comprising the amino acid sequence of SEQ ID NO: 154, a vlCDR2 comprising the amino acid sequence of SEQ ID NO: 155, and a vlCDR3 comprising the amino acid sequence of SEQ ID NO: 156. In some such combinations, the VH1 and VL1 comprise a variable heavy domain comprising a vhCDR1 comprising the amino acid sequence of SEQ ID NO: 118, a vhCDR2 comprising the amino acid sequence of SEQ ID NO: 158, and a vhCDR3 comprising the amino acid sequence of SEQ ID NO: 120, and a variable light domain comprising a vlCDR1 comprising the amino acid sequence of SEQ ID NO: 122, a vlCDR2 comprising the amino acid sequence of SEQ ID NO: 123, and a vlCDR3 comprising the amino acid sequence of SEQ ID NO: 160. In some such combinations, the VH1 and VL1 comprise a variable heavy domain comprising a vhCDR1 comprising the amino acid sequence of SEQ ID NO: 162, a vhCDR2 comprising the amino acid sequence of SEQ ID NO: 163, and a vhCDR3 comprising the amino acid sequence of SEQ ID NO: 164, and a variable light domain comprising a vlCDR1 comprising the amino acid sequence of SEQ ID NO: 166, a vlCDR2 comprising the amino acid sequence of SEQ ID NO: 167, and a vlCDR3 comprising the amino acid sequence of SEQ ID NO: 168. In some such combinations, the VH1 and VL1 comprise a variable heavy domain comprising a vhCDR1 comprising the amino acid sequence of SEQ ID NO: 170, a vhCDR2 comprising the amino acid sequence of SEQ ID NO: 171, and a vhCDR3 comprising the amino acid sequence of SEQ ID NO: 172, and a variable light domain comprising a vlCDR1 comprising the amino acid sequence of SEQ ID NO: 174, a vlCDR2 comprising the amino acid sequence of SEQ ID NO: 175, and a vlCDR3 comprising the amino acid sequence of SEQ ID NO: 176.

In some such combinations, the VH1 is at least 90%, at least 95%, at least 96%, at least 97%, at least 98%, or at least 99% identical to the amino acid sequence of any one of SEQ ID NOS: 2241, 2253, 2259, 1674, and 1200, and the VL1 is at least 90%, at least 95%, at least 96%, at least 97%, at least 98%, or at least 99% identical to the amino acid sequence of any one of SEQ ID NOS: 2243 and 1204. In some such combinations, the VH1 is at least 90%, at least 95%, at least 96%, at least 97%, at least 98%, or at least 99% identical to the amino acid sequence of SEQ ID NO: 2241, and the VL1 is at least 90%, at least 95%, at least 96%, at least 97%, at least 98%, or at least 99% identical to the amino acid sequence of SEQ ID NO: 2243. In some such combinations, the VH1 is at least 90%, at least 95%, at least 96%, at least 97%, at least 98%, or at least 99% identical to the amino acid sequence of SEQ ID NO: 2253, and the VL1 is at least 90%, at least 95%, at least 96%, at least 97%, at least 98%, or at least 99% identical to the amino acid sequence of SEQ ID NO: 2243. In some such combinations, the VH1 is at least 90%, at least 95%, at least 96%, at least 97%, at least 98%, or at least 99% identical to the amino acid sequence of SEQ ID NO: 2259, and the VL1 is at least 90%, at least 95%, at least 96%, at least 97%, at least 98%, or at least 99% identical to the amino acid sequence of SEQ ID NO: 2243. In some such combinations, the VH1 is at least 90%, at least 95%, at least 96%, at least 97%, at least 98%, or at least 99% identical to the amino acid sequence of SEQ ID NO: 1674, and the VL1 is at least 90%, at least 95%, at least 96%, at least 97%, at least 98%, or at least 99% identical to the amino acid sequence of SEQ ID NO: 1204. In some such combinations, the VH1 is at least 90%, at least 95%, at least 96%, at least 97%, at least 98%, or at least 99% identical to the amino acid sequence of SEQ ID NO: 1200, and the VL1 is at least 90%, at least 95%, at least 96%, at least 97%, at least 98%, or at least 99% identical to the amino acid sequence of SEQ ID NO: 1204. In some such combinations, the VH1 is at least 90%, at least 95%, at least 96%, at least 97%, at least 98%, or at least 99% identical to the amino acid sequence of any one of SEQ ID NOS: 1427-1823 and 2316-2330, and the VL1 is at least 90%, at least 95%, at least 96%, at least 97%, at least 98%, or at least 99% identical to the amino acid sequence of any one of SEQ ID NOS: 1824-1923. In some such combinations, the VH1 is at least 90%, at least 95%, at least 96%, at least 97%, at least 98%, or at least 99% identical to the amino acid sequence of any one of SEQ ID NOS: 1674, 1200, 1661, and 2323, and the VL1 is at least 90%, at least 95%, at least 96%, at least 97%, at least 98%, or at least 99% identical to the amino acid sequence of SEQ ID NO: 1204. In some such combinations, the VH1 is at least 90%, at least 95%, at least 96%, at least 97%, at least 98%, or at least 99% identical to the amino acid sequence of SEQ ID NO: 1661, and the VL1 is at least 90%, at least 95%, at least 96%, at least 97%, at least 98%, or at least 99% identical to the amino acid sequence of SEQ ID NO: 1204. In some such combinations, the VH1 is at least 90%, at least 95%, at least 96%, at least 97%, at least 98%, or at least 99% identical to the amino acid sequence of SEQ ID NO: 2323, and the VL1 is at least 90%, at least 95%, at least 96%, at least 97%, at least 98%, or at least 99% identical to the amino acid sequence of SEQ ID NO: 1204. In some such combinations, the VH1 is at least 90%, at least 95%, at least 96%, at least 97%, at least 98%, or at least 99% identical to the amino acid sequence of SEQ ID NO: 101, and the VL1 is at least 90%, at least 95%, at least 96%, at least 97%, at least 98%, or at least 99% identical to the amino acid sequence of SEQ ID NO: 105. In some such combinations, the VH1 is at least 90%, at least 95%, at least 96%, at least 97%, at least 98%, or at least 99% identical to the amino acid sequence of SEQ ID NO: 109, and the VL1 is at least 90%, at least 95%, at least 96%, at least 97%, at least 98%, or at least 99% identical to the amino acid sequence of SEQ ID NO: 113. In some such combinations, the VH1 is at least 90%, at least 95%, at least 96%, at least 97%, at least 98%, or at least 99% identical to the amino acid sequence of SEQ ID NO: 117, and the VL1 is at least 90%, at least 95%, at least 96%, at least 97%, at least 98%, or at least 99% identical to the amino acid sequence of SEQ ID NO: 121. In some such combinations, the VH1 is at least 90%, at least 95%, at least 96%, at least 97%, at least 98%, or at least 99% identical to the amino acid sequence of SEQ ID NO: 125, and the VL1 is at least 90%, at least 95%, at least 96%, at least 97%, at least 98%, or at least 99% identical to the amino acid sequence of SEQ ID NO: 129. In some such combinations, the VH1 is at least 90%, at least 95%, at least 96%, at least 97%, at least 98%, or at least 99% identical to the amino acid sequence SEQ ID NO: 133, and the VL1 is at least 90%, at least 95%, at least 96%, at least 97%, at least 98%, or at least 99% identical to the amino acid sequence of SEQ ID NO: 137. In some such combinations, the VH1 is at least 90%, at least 95%, at least 96%, at least 97%, at least 98%, or at least 99% identical to the amino acid sequence of SEQ ID NO: 141, and the VL1 is at least 90%, at least 95%, at least 96%, at least 97%, at least 98%, or at least 99% identical to the amino acid sequence of SEQ ID NO: 145. In some such combinations, the VH1 is at least 90%, at least 95%, at least 96%, at least 97%, at least 98%, or at least 99% identical to the amino acid sequence of SEQ ID NO: 149, and the VL1 is at least 90%, at least 95%, at least 96%, at least 97%, at least 98%, or at least 99% identical to the amino acid sequence of SEQ ID NO: 153. In some such combinations, the VH1 is at least 90%, at least 95%, at least 96%, at least 97%, at least 98%, or at least 99% identical to the amino acid sequence of SEQ ID NO: 157, and the VL1 is at least 90%, at least 95%, at least 96%, at least 97%, at least 98%, or at least 99% identical to the amino acid sequence of SEQ ID NO: 159. In some such combinations, the VH1 is at least 90%, at least 95%, at least 96%, at least 97%, at least 98%, or at least 99% identical to the amino acid sequence of SEQ ID NO: 161, and the VL1 is at least 90%, at least 95%, at least 96%, at least 97%, at least 98%, or at least 99% identical to the amino acid sequence of SEQ ID NO: 165. In some such combinations, the VH1 is at least 90%, at least 95%, at least 96%, at least 97%, at least 98%, or at least 99% identical to the amino acid sequence of SEQ ID NO: 169, and the VL1 is at least 90%, at least 95%, at least 96%, at least 97%, at least 98%, or at least 99% identical to the amino acid sequence of SEQ ID NO: 173. In some such combinations, the VH1 and the VL1 are at least 90%, at least 95%, at least 96%, at least 97%, at least 98%, or at least 99% identical to any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such combinations, the VH1 and the VL1 are at least 90%, at least 95%, at least 96%, at least 97%, at least 98%, or at least 99% identical to any pair of anti-TL1A variable heavy domain and variable light domain in Tables 29 and 31-34.

In some such combinations, the VH1 comprises the amino acid sequence of any one of SEQ ID NOS: 2241, 2253, 2259, 1674, and 1200, and the VL1 comprises the amino acid sequence of any one of SEQ ID NOS: 2243 and 1204. In some such combinations, the VH1 comprises the amino acid sequence of SEQ ID NO: 2241, and the VL1 comprises the amino acid sequence of SEQ ID NO: 2243. In some such combinations, the VH1 comprises the amino acid sequence of SEQ ID NO: 2253, and the VL1 comprises the amino acid sequence of SEQ ID NO: 2243. In some such combinations, the VH1 comprises the amino acid sequence of SEQ ID NO: 2259, and the VL1 comprises the amino acid sequence of SEQ ID NO: 2243. In some such combinations, the VH1 comprises the amino acid sequence of SEQ ID NO: 1674, and the VL1 comprises the amino acid sequence of SEQ ID NO: 1204. In some such combinations, the VH1 comprises the amino acid sequence of SEQ ID NO: 1200, and the VL1 comprises the amino acid sequence of SEQ ID NO: 1204. In some such combinations, the VH1 comprises the amino acid sequence of any one of SEQ ID NOS: 1427-1823 and 2316-2330, and the VL1 comprises the amino acid sequence of any one of SEQ ID NOS: 1824-1923. In some such combinations, the VH1 comprises the amino acid sequence of any one of SEQ ID NOS: 1674, 1200, 1661, and 2323, and the VL1 comprises the amino acid sequence of SEQ ID NO: 1204. In some such combinations, the VH1 comprises the amino acid sequence of SEQ ID NO: 1661, and the VL1 comprises the amino acid sequence of SEQ ID NO: 1204. In some such combinations, the VH1 comprises the amino acid sequence of SEQ ID NO: 2323, and the VL1 comprises the amino acid sequence of SEQ ID NO: 1204. In some such combinations, the VH1 comprises the amino acid sequence of SEQ ID NO: 101, and the VL1 comprises the amino acid sequence of SEQ ID NO: 105, optionally wherein the heavy chain comprises the amino acid sequence of SEQ ID NO: 1419 or a variant thereof in which the N-terminal glutamine is replaced with pyroglutamate and/or the C-terminal lysine or the C-terminal lysine and glycine are removed and the light chain comprises the amino acid sequence of SEQ ID NO: 1420. In some such combinations, the VH1 comprises the amino acid sequence of SEQ ID NO: 109, and the VL1 comprises the amino acid sequence of SEQ ID NO: 113. In some such combinations, the VH1 comprises the amino acid sequence of SEQ ID NO: 117, and the VL1 comprises the amino acid sequence of SEQ ID NO: 121. In some such combinations, the VH1 comprises the amino acid sequence of SEQ ID NO: 125, and the VL1 comprises the amino acid sequence of SEQ ID NO: 129. In some such combinations, the VH1 comprises the amino acid sequence of SEQ ID NO: 133, and the VL1 comprises the amino acid sequence of SEQ ID NO: 137. In some such combinations, the VH1 comprises the amino acid sequence of SEQ ID NO: 141, and the VL1 comprises the amino acid sequence of SEQ ID NO: 145. In some such combinations, the VH1 comprises the amino acid sequence of SEQ ID NO: 149, and the VL1 comprises the amino acid sequence of SEQ ID NO: 153. In some such combinations, the VH1 comprises the amino acid sequence of SEQ ID NO: 157, and the VL1 comprises the amino acid sequence of SEQ ID NO: 159. In some such combinations, the VH1 comprises the amino acid sequence of SEQ ID NO: 161, and the VL1 comprises the amino acid sequence of SEQ ID NO: 165. In some such combinations, the VH1 comprises the amino acid sequence of SEQ ID NO: 169, and the VL1 comprises the amino acid sequence of SEQ ID NO: 173. In some such combinations, the VH1 and the VL1 comprise any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such combinations, the VH1 and the VL1 comprise any pair of anti-TL1A variable heavy domain and variable light domain in Tables 29 and 31-34. In some such combinations, the VH1 or VL1 can comprise variants of any of the above in which an N-terminal glutamine or glutamate is replaced with pyroglutamate.

In some such combinations, the VH2 and VL2 comprise a variable heavy domain comprising a vhCDR1, a vhCDR2, and a vhCDR3 contained within a variable heavy domain comprising the amino acid sequence of any one of SEQ ID NOS: 2233, 2235, and 179, and a variable light domain comprising a vlCDR1, a vlCDR2, and a vlCDR3 contained within a variable light domain comprising the amino acid sequence of any one of SEQ ID NOS: 2234, 2237, and 1248. In some such combinations, the VH2 and VL2 comprise a variable heavy domain comprising a vhCDR1, a vhCDR2, and a vhCDR3 contained within a variable heavy domain comprising the amino acid sequence of SEQ ID NO: 2233, and a variable light domain comprising a vlCDR1, a vlCDR2, and a vlCDR3 contained within a variable light domain comprising the amino acid sequence of SEQ ID NO: 2234. In some such combinations, the VH2 and VL2 comprise a variable heavy domain comprising a vhCDR1, a vhCDR2, and a vhCDR3 contained within a variable heavy domain comprising the amino acid sequence of SEQ ID NO: 2235, and a variable light domain comprising a vlCDR1, a vlCDR2, and a vlCDR3 contained within a variable light domain comprising the amino acid sequence of SEQ ID NO: 2237. In some such combinations, the VH2 and VL2 comprise a variable heavy domain comprising a vhCDR1, a vhCDR2, and a vhCDR3 contained within a variable heavy domain comprising the amino acid sequence of SEQ ID NO: 179, and a variable light domain comprising a vlCDR1, a vlCDR2, and a vlCDR3 contained within a variable light domain comprising the amino acid sequence of SEQ ID NO: 1248. In some such combinations, the VH2 and VL2 comprise a variable heavy domain comprising a vhCDR1, a vhCDR2, and a vhCDR3 contained within a variable heavy domain comprising the amino acid sequence of SEQ ID NO: 2113, and a variable light domain comprising a vlCDR1, a vlCDR2, and a vlCDR3 contained within a variable light domain comprising the amino acid sequence of SEQ ID NO: 2231. In some such combinations, the VH2 and VL2 comprise a variable heavy domain comprising a vhCDR1, a vhCDR2, and a vhCDR3 contained within a variable heavy domain comprising the amino acid sequence of SEQ ID NO: 1967, and a variable light domain comprising a vlCDR1, a vlCDR2, and a vlCDR3 contained within a variable light domain comprising the amino acid sequence of SEQ ID NO: 1248. In some such combinations, the VH2 and VL2 comprise a variable heavy domain comprising a vhCDR1, a vhCDR2, and a vhCDR3 and a variable light domain comprising a vlCDR1, a vlCDR2, and a vlCDR3 contained within a variable heavy domain and variable light domain pair in XENP53412, XENP53413, XENP53369, XENP53375, XENP53387, XENP53415, XENP53370, XENP53371, XENP53376, XENP53377, XENP53378, XENP53379, XENP53380, or XENP53414. Optionally, the CDRs in any of the above are as defined by the Kabat, Chothia, Kabat+Chothia, AbM, contact, or IMGT definition.

In some such combinations, the VH2 and VL2 comprise a variable heavy domain comprising a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182 or 2236, and a variable light domain comprising a vlCDR1 comprising the amino acid sequence of SEQ ID NO: 184, a vlCDR2 comprising the amino acid sequence of SEQ ID NO: 1249 or 2238, and a vlCDR3 comprising the amino acid sequence of SEQ ID NO: 1250. In some such combinations, the VH2 and VL2 comprise a variable heavy domain comprising a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and a variable light domain comprising a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such combinations, the VH2 and VL2 comprise a variable heavy domain comprising a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 2236, and a variable light domain comprising a vlCDR1 comprising the amino acid sequence of SEQ ID NO: 184, a vlCDR2 comprising the amino acid sequence of SEQ ID NO: 2238, and a vlCDR3 comprising the amino acid sequence of SEQ ID NO: 1250. In some such combinations, the VH2 and VL2 comprise a variable heavy domain comprising a vhCDR1 comprising the amino acid sequence of SEQ ID NO: 2239, a vhCDR2 comprising the amino acid sequence of SEQ ID NO: 181, and a vhCDR3 comprising the amino acid sequence of SEQ ID NO: 182, and a variable light domain comprising a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250.

In some such combinations, the VH2 is at least 90%, at least 95%, at least 96%, at least 97%, at least 98%, or at least 99% identical to the amino acid sequence of any one of SEQ ID NOS: 2233, 2235, and 179, and the VL2 is at least 90%, at least 95%, at least 96%, at least 97%, at least 98%, or at least 99% identical to the amino acid sequence of any one of SEQ ID NOS: 2234, 2237, and 1248. In some such combinations, the VH2 is at least 90%, at least 95%, at least 96%, at least 97%, at least 98%, or at least 99% identical to the amino acid sequence of SEQ ID NO: 2233, and the VL2 is at least 90%, at least 95%, at least 96%, at least 97%, at least 98%, or at least 99% identical to the amino acid sequence of SEQ ID NO: 2234. In some such combinations, the VH2 is at least 90%, at least 95%, at least 96%, at least 97%, at least 98%, or at least 99% identical to the amino acid sequence of SEQ ID NO: 2235, and the VL2 is at least 90%, at least 95%, at least 96%, at least 97%, at least 98%, or at least 99% identical to the amino acid sequence of SEQ ID NO: 2237. In some such combinations, the VH2 is at least 90%, at least 95%, at least 96%, at least 97%, at least 98%, or at least 99% identical to the amino acid sequence of SEQ ID NO: 179, and the VL2 is at least 90%, at least 95%, at least 96%, at least 97%, at least 98%, or at least 99% identical to the amino acid sequence of SEQ ID NO: 1248. In some such combinations, the VH2 is at least 90%, at least 95%, at least 96%, at least 97%, at least 98%, or at least 99% identical to the amino acid sequence of SEQ ID NO: 2113, and the VL2 is at least 90%, at least 95%, at least 96%, at least 97%, at least 98%, or at least 99% identical to the amino acid sequence of SEQ ID NO: 2231. In some such combinations, the VH2 is at least 90%, at least 95%, at least 96%, at least 97%, at least 98%, or at least 99% identical to the amino acid sequence of SEQ ID NO: 1967, and the VL2 is at least 90%, at least 95%, at least 96%, at least 97%, at least 98%, or at least 99% identical to the amino acid sequence of SEQ ID NO: 1248. In some such combinations, the VH2 is at least 90%, at least 95%, at least 96%, at least 97%, at least 98%, or at least 99% identical to the amino acid sequence of any one of SEQ ID NOS: 1927-2132, and the VL2 is at least 90%, at least 95%, at least 96%, at least 97%, at least 98%, or at least 99% identical to the amino acid sequence of any one of SEQ ID NOS: 2133-2232. In some such combinations, the VH2 and the VL2 are at least 90%, at least 95%, at least 96%, at least 97%, at least 98%, or at least 99% identical to any pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such combinations, the VH2 and the VL2 are at least 90%, at least 95%, at least 96%, at least 97%, at least 98%, or at least 99% identical to any pair of anti-IL23 variable heavy domain and variable light domain in Table 29 or 33.

In some such combinations, the VH2 comprises the amino acid sequence of any one of SEQ ID NOS: 2233, 2235, and 179, and the VL2 comprises the amino acid sequence of any one of SEQ ID NOS: 2234, 2237, and 1248. In some such combinations, the VH2 comprises the amino acid sequence of SEQ ID NO: 2233, and the VL2 comprises the amino acid sequence of SEQ ID NO: 2234. In some such combinations, the VH2 comprises the amino acid sequence of SEQ ID NO: 2235, and the VL2 comprises the amino acid sequence of SEQ ID NO: 2237. In some such combinations, the VH2 comprises the amino acid sequence of SEQ ID NO: 179, and the VL2 comprises the amino acid sequence of SEQ ID NO: 1248. In some such combinations, the VH2 comprises the amino acid sequence of SEQ ID NO: 2113, and the VL2 comprises the amino acid sequence of SEQ ID NO: 2231. In some such combinations, the VH2 comprises the amino acid sequence of SEQ ID NO: 1967, and the VL2 comprises the amino acid sequence of SEQ ID NO: 1248. In some such combinations, the VH2 comprises the amino acid sequence of any one of SEQ ID NOS: 1927-2132, and the VL2 comprises the amino acid sequence of any one of SEQ ID NOS: 2133-2232. In some such combinations, the VH2 and the VL2 comprise any pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some such combinations, the VH2 and the VL2 comprise any pair of anti-IL23 variable heavy domain and variable light domain in Table 29 or 33. In some such combinations, the VH2 or VL2 can comprise variants of any of the above in which an N-terminal glutamine or glutamate is replaced with pyroglutamate.

In some such combinations, (i) the VH1 comprises a vhCDR1, a vhCDR2, and a vhCDR3 contained within a variable heavy domain comprising the amino acid sequence of any one of SEQ ID NOS: 2241, 2253, 2259, 1674, and 1200, (ii) the VL1 comprises a vlCDR1, a vlCDR2, and a vlCDR3 contained within a variable light domain comprising the amino acid sequence of any one of SEQ ID NOS: 2243 and 1204, (iii) the VH2 comprises a vhCDR1, a vhCDR2, and a vhCDR3 contained within a variable heavy domain comprising the amino acid sequence of any one of SEQ ID NOS: 2233, 2235, and 179, and (iv) the VL2 comprises a vlCDR1, a vlCDR2, and a vlCDR3 contained within a variable light domain comprising the amino acid sequence of any one of SEQ ID NOS: 2234, 2237, and 1248, optionally wherein the CDRs are as defined by the Kabat, Chothia, Kabat+Chothia, AbM, contact, or IMGT definition. In some such combinations, (i) the VH1 comprises a vhCDR1 comprising the amino acid sequence of SEQ ID NO: 102 or 1201, a vhCDR2 comprising the amino acid sequence of SEQ ID NO: 1924 or 1202, and a vhCDR3 comprising the amino acid sequence of SEQ ID NO: 1203 or 104, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182 or 2236, and (iv) the VL2 comprises a vlCDR1 comprising the amino acid sequence of SEQ ID NO: 184, a vlCDR2 comprising the amino acid sequence of SEQ ID NO: 1249 or 2238, and a vlCDR3 comprising the amino acid sequence of SEQ ID NO: 1250. In some such combinations, (i) the VH1 comprises the amino acid sequence of any one of SEQ ID NOS: 2241, 2253, 2259, 1674, and 1200, (ii) the VL1 comprises the amino acid sequence of any one of SEQ ID NOS: 2243 and 1204, (iii) the VH2 comprises the amino acid sequence of any one of SEQ ID NOS: 2233, 2235, and 179, and (iv) the VL2 comprises the amino acid sequence of any one of SEQ ID NOS: 2234, 2237, and 1248, or variants thereof in which an N-terminal glutamine or glutamate is replaced with pyroglutamate.

In some such combinations, (i) the VH1 comprises a vhCDR1, a vhCDR2, and a vhCDR3 contained within a variable heavy domain comprising the amino acid sequence of SEQ ID NO: 2241, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234, optionally wherein the CDRs are as defined by the Kabat, Chothia, Kabat+Chothia, AbM, contact, or IMGT definition. In some such combinations, (i) the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such combinations, (i) the VH1 comprises the amino acid sequence of SEQ ID NO: 2241, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234, or variants thereof in which an N-terminal glutamine or glutamate is replaced with pyroglutamate.

In some such combinations, (i) the VH1 comprises a vhCDR1, a vhCDR2, and a vhCDR3 contained within a variable heavy domain comprising the amino acid sequence of SEQ ID NO: 2241, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2235, and (iv) the VL2 comprises a vlCDR1, a vlCDR2, and a vlCDR3 contained within a variable light domain comprising the amino acid sequence of SEQ ID NO: 2237. In some such combinations, (i) the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 2236, and (iv) the VL2 comprises a vlCDR1 comprising the amino acid sequence of SEQ ID NO: 184, a vlCDR2 comprising the amino acid sequence of SEQ ID NO: 2238, and a vlCDR3 comprising the amino acid sequence of SEQ ID NO: 1250. In some such combinations, (i) the VH1 comprises the amino acid sequence of SEQ ID NO: 2241, (ii) the VL1 comprises the amino acid sequence of SEQ ID NO: 2243, (iii) the VH2 comprises the amino acid sequence of SEQ ID NO: 2235, and (iv) the VL2 comprises the amino acid sequence of SEQ ID NO: 2237, or variants thereof in which an N-terminal glutamine or glutamate is replaced with pyroglutamate.

In some such combinations, (i) the VH1 comprises a vhCDR1, a vhCDR2, and a vhCDR3 contained within a variable heavy domain comprising the amino acid sequence of SEQ ID NO: 2253, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234, optionally wherein the CDRs are as defined by the Kabat, Chothia, Kabat+Chothia, AbM, contact, or IMGT definition. In some such combinations, (i) the VH1 comprises a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such combinations, (i) the VH1 comprises the amino acid sequence of SEQ ID NO: 2253, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234, or variants thereof in which an N-terminal glutamine or glutamate is replaced with pyroglutamate.

In some such combinations, (i) the VH1 comprises a vhCDR1, a vhCDR2, and a vhCDR3 contained within a variable heavy domain comprising the amino acid sequence of SEQ ID NO: 2259, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234, optionally wherein the CDRs are as defined by the Kabat, Chothia, Kabat+Chothia, AbM, contact, or IMGT definition. In some such combinations, (i) the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 104, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such combinations, (i) the VH1 comprises the amino acid sequence of SEQ ID NO: 2259, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234, or variants thereof in which an N-terminal glutamine or glutamate is replaced with pyroglutamate.

In some such combinations, (i) the VH1 comprises a vhCDR1, a vhCDR2, and a vhCDR3 contained within a variable heavy domain comprising the amino acid sequence of SEQ ID NO: 1674, (ii) the VL1 comprises a vlCDR1, a vlCDR2, and a vlCDR3 contained within a variable light domain comprising the amino acid sequence of SEQ ID NO: 1204, (iii) the VH2 comprises a vhCDR1, a vhCDR2, and a vhCDR3 contained within a variable heavy domain comprising the amino acid sequence of SEQ ID NO: 179, and (iv) the VL2 comprises a vlCDR1, a vlCDR2, and a vlCDR3 contained within a variable light domain comprising the amino acid sequence of SEQ ID NO: 1248, optionally wherein the CDRs are as defined by the Kabat, Chothia, Kabat+Chothia, AbM, contact, or IMGT definition. In some such combinations, (i) the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such combinations, (i) the VH1 comprises the amino acid sequence of SEQ ID NO: 1674, (ii) the VL1 comprises the amino acid sequence of SEQ ID NO: 1204, (iii) the VH2 comprises the amino acid sequence of SEQ ID NO: 179, and (iv) the VL2 comprises the amino acid sequence of SEQ ID NO: 1248, or variants thereof in which an N-terminal glutamine or glutamate is replaced with pyroglutamate.

In some such combinations, (i) the VH1 comprises a vhCDR1, a vhCDR2, and a vhCDR3 contained within a variable heavy domain comprising the amino acid sequence of SEQ ID NO: 1200, (ii) the VL1 comprises a vlCDR1, a vlCDR2, and a vlCDR3 contained within a variable light domain comprising the amino acid sequence of SEQ ID NO: 1204, (iii) the VH2 comprises a vhCDR1, a vhCDR2, and a vhCDR3 contained within a variable heavy domain comprising the amino acid sequence of SEQ ID NO: 179, and (iv) the VL2 comprises a vlCDR1, a vlCDR2, and a vlCDR3 contained within a variable light domain comprising the amino acid sequence of SEQ ID NO: 1248, optionally wherein the CDRs are as defined by the Kabat, Chothia, Kabat+Chothia, AbM, contact, or IMGT definition. In some such combinations, (i) the VH1 comprises a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some such combinations, (i) the VH1 comprises the amino acid sequence SEQ ID NO: 1200, (ii) the VL1 comprises the amino acid sequence of SEQ ID NO: 1204, (iii) the VH2 comprises the amino acid sequence of SEQ ID NO: 179, and (iv) the VL2 comprises the amino acid sequence of SEQ ID NO: 1248, or variants thereof in which an N-terminal glutamine or glutamate is replaced with pyroglutamate.

In some such combinations, the first antibody comprises (i) a first heavy chain comprising the VH1 and a first heavy chain constant domain, and (ii) a first light chain comprising the VL1 and a first light chain constant domain, and wherein the second antibody comprises (iii) a second heavy chain comprising the VH2 and a second heavy chain constant domain, and (iv) a second light chain comprising the VL2 and a second light chain constant domain. In some such combinations, the first heavy chain constant domain and the second heavy chain constant domain each comprise one or more ablation variants. In some such combinations, the first heavy chain constant domain or the second heavy chain constant domain comprises one or more pI variants. In some such combinations, the first heavy chain constant domain comprises amino acid substitutions E233P/L234V/L235A/G236del/S267K and/or the second heavy chain constant domain comprises amino acid substitutions E233P/L234V/L235A/G236del/S267K, wherein numbering is according to EU numbering. In some such combinations, the first heavy chain constant domain comprises amino acid substitutions M428L/N434S and/or the second heavy chain constant domain comprises amino acid substitutions M428L/N434S, wherein numbering is according to EU numbering. In some such combinations, the first heavy chain constant domain comprises amino acid substitutions E233P/L234V/L235A/G236del/S267K and M428L/N434S and/or the second heavy chain constant domain comprises amino acid substitutions E233P/L234V/L235A/G236del/S267K and M428L/N434S, wherein numbering is according to EU numbering.

In some such combinations, the first heavy chain constant domain is of isotype IgG1, and/or the second heavy chain constant domain is of isotype IgG1. In some such combinations, the first antibody is a chimeric or humanized antibody, and/or the second antibody is a chimeric or humanized antibody.

In some such combinations, the first antibody specifically binds human TL1A with a K_{D} of less than about 6.5E-11 M, less than about 6.4 E-11 M, or less than about 6.3E-11 as measured by surface plasmon resonance or Biacore at 25° C., optionally wherein the first antibody specifically binds human TL1A with a K_{D} of less than about 3E-11 M, less than about 2.9E-11 M, less than about 2.8E-11 M, or less than about 2.7E-11 M. In some such combinations, the first antibody inhibits TL1A activity in a TL1A luciferase reporter assay with an IC₅₀ of less than about 4.5 nM. In some such combinations, the second antibody specifically binds human IL23 with a K_{D} of less than about 1.1E-10 M as measured by surface plasmon resonance or Biacore at 25° C., optionally wherein the second antibody specifically binds human IL23 with a K_{D} of less than about 6E-11 M, less than about 5.9E-11 M, less than about 5.8E-11 M, or less than about 5.7E-11 M. In some such combinations, the second antibody inhibits IL23 activity in an IL23 luciferase reporter assay with an IC₅₀ of less than about 65 pM, less than about 64 pM, or less than about 63 pM, optionally wherein the second antibody inhibits IL23 activity with an IC₅₀ of less than about 60 pM, less than about 59 pM, less than about 58 pM, less than about 57 pM, or less than about 56 pM.

In another aspect, provided are pharmaceutical compositions comprising any of the above combinations and a pharmaceutically acceptable carrier. In another aspect, provided is a kit comprising the combination of any of the above combinations or pharmaceutical compositions. In another aspect, provided is a vessel or delivery device comprising any of the above combinations or pharmaceutical compositions. In another aspect, provided is a nucleic acid composition comprising a nucleic acid or a set of nucleic acids encoding any of the above combinations. In another aspect, provided is an expression vector composition comprising an expression vector or a set of expression vectors comprising the nucleic acid composition. In another aspect, provided is a method of inhibiting or reducing TL1A-mediated activity and/or IL23-mediated activity in a subject in need thereof, comprising administering to the subject the combination of any of the above combinations or pharmaceutical compositions. In another aspect, provided are any of the above combinations or pharmaceutical compositions for use in inhibiting or reducing TL1A-mediated activity and/or IL23-mediated activity in a subject in need thereof. In another aspect, provided is use of any of the above combinations or pharmaceutical compositions for inhibiting or reducing TL1A-mediated activity and/or IL23-mediated activity in a subject in need thereof. In another aspect, provided is use of any of the above combinations or pharmaceutical compositions in the manufacture of a medicament for inhibiting or reducing TL1A-mediated activity and/or IL23-mediated activity in a subject in need thereof.

In another aspect, provided is a method of treating a TL1A-associated disease and/or IL23-associated disease in a subject in need thereof, comprising administering to the subject any of the above combinations or pharmaceutical compositions. In another aspect, provided are any of the above combinations or pharmaceutical compositions for use in the treatment of a TL1A-associated disease and/or IL23-associated disease in a subject in need thereof. In another aspect, provided is use of any of the above combinations or pharmaceutical compositions for the treatment of a TL1A-associated disease and/or IL23-associated disease in a subject in need thereof. In another aspect, provided is use of any of the above combinations or pharmaceutical compositions in the manufacture of a medicament for the treatment of a TL1A-associated disease and/or IL23-associated disease in a subject in need thereof. In some such methods, combinations or compositions for use, or uses, the TL1A-associated disease and/or IL23-associated disease is an inflammatory disease, an autoimmune disease, or a fibrotic disease, optionally wherein the TL1A-associated disease and/or IL23-associated disease is an inflammatory bowel disease. In some such methods, combinations or compositions for use, or uses, the subject is a human.

In another aspect, provided is a heterodimeric antibody comprising: (a) a first monomer comprising: (i) a single chain variable fragment (scFv); and (ii) a first Fc domain, wherein the scFv is covalently attached to the N-terminus of the first Fc domain using a domain linker; (b) a second monomer comprising, from N-terminal to C-terminal, a VH1-CH1-hinge-CH2-CH3, wherein VH1 is a first variable heavy domain and CH2-CH3 is a second Fc domain; and (c) a light chain comprising, from N-terminal to C-terminal, VL1-CL, wherein VL1 is a first variable light domain and CL is a constant light domain, wherein the scFv comprises a second VH domain (VH2), a scFv linker, and a second variable light domain (VL2), wherein the VH1 and the VL1 together form a first antigen-binding domain (ABD) and the VH2 and the VL2 together form a second ABD, and wherein one of the first ABD and second ABD is a TL1A binding domain and the other of the first ABD and second ABD is a IL23 binding domain.

In another aspect, provided is a heterodimeric antibody comprising: (a) a first monomer comprising a VH1-CH1-hinge-CH2-CH3, wherein VH1 is a first variable heavy domain and CH2-CH3 is a first Fc domain; (b) a second monomer comprising a VH2-CH1-hinge-CH2-CH3, wherein VH2 is a second variable heavy domain and CH2-CH3 is a second Fc domain; (c) a first common light chain comprising a VL-CL; and (d) a second common light chain comprising a VL-CL, wherein the VH1 and the VL of the first common light chain together form a first antigen-binding domain (ABD) and the VH2 and the VL of the second common light chain together form a second ABD.

In another aspect, provided is a heterodimeric antibody comprising: (a) a first monomer comprising: (i) a VH1-CH1-hinge-CH2-CH3, and (ii) a first scFv comprising a VH2-scFv linker-VL2, wherein the scFv is covalently attached to the CH3 of the first monomer by a linker; (b) a second monomer comprising: (i) a VH1-CH1-hinge-CH2-CH3, and (ii) a second scFv comprising a VH2-scFv linker-VL2, wherein the scFv is covalently attached to the CH3 of the second monomer by a linker; (c) a first light chain comprising a VL1-CL; and (d) a second light chain comprising a VL1-CL, wherein each of the VH1s of the first monomer and second monomer are a first variable heavy domain, each of the VL1s of the first monomer and second monomer are a first light domain, each of the VH2s are a second variable heavy domain, and each of the VL2s are a second variable light domain, where the VH1 of the first monomer and VL1 of the first light chain, and the VH1 of the second monomer and VL1 of the second light chain each form a first antigen-binding domain, and the VH2 and VL2 of the first and second scFvs each for a second antigen-binding domain, wherein the first ABDs or the second ABDs are TL1A binding domains and the other of the first ABDs and second ABDs are IL23 binding domains.

### BRIEF DESCRIPTION OF THE DRAWINGS

FIG. 1 panels A-R depict bispecific formats of the present invention. Panel A depicts the “1+1 Fab-scFv-Fc” format, with a first Fab arm binding a first antigen and a second scFv arm binding second antigen. The 1+1 Fab-scFv-Fc format comprises a first monomer comprising a first heavy chain variable region (VH1) covalently attached to the N-terminus of a first heterodimeric Fc backbone (optionally via a linker), a second monomer comprising a single-chain Fv covalently attached to the N-terminus of a second corresponding heterodimeric Fc backbone (optionally via a linker), and a third monomer comprising a light chain variable region covalently to a light chain constant domain, wherein the light chain variable region is complementary to the VH1. Panel B depicts the “2+1 Fab₂-scFv-Fc” format, with a first Fab arm and a second Fab-scFv arm, wherein the Fab binds a first antigen and the scFv binds second antigen. The 2+1 Fab₂-scFv-Fc format comprises a first monomer comprising a first heavy chain variable region (VH1) covalently attached to the N-terminus of a first heterodimeric Fc backbone (optionally via a linker), a second monomer comprising the VH1 covalently attached (optionally via a linker) to a single-chain Fv covalently attached (optionally via a linker) to the N-terminus of a second corresponding heterodimeric Fc backbone, and a third monomer comprising a light chain variable region covalently to a light chain constant domain, wherein the light chain variable region is complementary to the VH1. Panel C depicts the “1+1 Common Light Chain” or “1+1 CLC” format, with a first Fc comprising a first Fab arm binding a first antigen and a second Fc comprising a second Fab arm binding second antigen. The 1+1 CLC format comprises a first monomer comprising VH1-CH1-hinge-CH2-CH3, a second monomer comprising VH2-CH1-hinge-CH2-CH3, and a third monomer comprising VL-CL. The VL pairs with the VH1 to form a binding domain with a first antigen binding specificity; and the VL pairs with the VH2 to form a binding domain with a second antigen binding specificity. Panel D depicts the “2+1 Common Light Chain” or “2+1 CLC” format, with a first Fc comprising 2 Fab arms each binding a first antigen and a second Fc comprising 1 Fab arm binding a second antigen. The 2+1 CLC format comprises a first monomer comprising VH1-CH1-hinge-VH1-CH1-hinge-CH2-CH3, a second monomer comprising VH2-CH1-hinge-CH2-CH3, and a third monomer comprising VL-CL. The VL pairs with the first and second VH1 to form binding domains with a first antigen binding specificity; and the VL pairs with the VH2 to form a binding domain with a second antigen binding specificity. Panel E depicts the “2+1 mAb-scFv” format, with a first Fc comprising an N-terminal Fab arm binding a first antigen and a second Fc comprising an N-terminal Fab arm binding the first antigen and a C-terminal scFv binding a second antigen. The 2+1 mAb-scFv format comprises a first monomer comprising VH1-CH1-hinge-CH2-CH3, a second monomer comprising VH1-CH1-hinge-CH2-CH3-scFv, and a third monomer comprising VL-CL. The VL pairs with the first and second VH1 to form binding domains with binding specificity for the first antigen. Panel F depicts the “2+1 Fab₂-Fc×scFv-Fc” format, with a first Fc comprising two N-terminal Fab arms binding a first antigen and a second Fc domain comprising an N-terminal scFv binding a second antigen. The 2+1 Fab₂-Fc×scFv-Fc format comprises a first monomer comprising VH1-CH1-VH2-CH1-hinge-CH2-CH3, a second monomer comprising scFv-domain linker-CH2-CH3, and a third and fourth monomer comprising VL-CL. The VL pairs with the VH1 and VH2 to form binding domains with binding specificity for the first antigen. Panel O depicts the “2+2 mAb-scFv” format, with a first Fc comprising an N-terminal Fab arm binding a first antigen and a C-terminal scFv binding a second antigen and a second Fc comprising an N-terminal Fab arm binding the first antigen and a C-terminal scFv binding a second antigen. The 2+2 mAb-scFv format comprises a first monomer comprising VH1-CH1-hinge-CH2-CH3-scFv, a second monomer comprising VH1-CH1-hinge-CH2-CH3-scFv, and a third monomer comprising VL-CL. The VL pairs with the first and second VH1 to form binding domains with binding specificity for the first antigen. Panel P depicts the “1+1 OrthoFab” format, with a first Fc comprising a first Fab arm binding a first antigen and a second Fc comprising a second Fab arm binding second antigen. The 1+1 OrthoFab format comprises a first monomer comprising VH1-CH1′-hinge-CH2-CH3, a second monomer comprising VH2-CH1”-hinge-CH2-CH3, a third monomer comprising VL1-CL1, and a fourth monomer comprising VL2-CL2. The VL1 pairs with the VH1 to form a binding domain with a first antigen binding specificity; and the VL2 pairs with the VH2 to form a binding domain with a second antigen binding specificity. The VH1 and VL1 include complementary electrostatic variants and the VH2 and VL2 include complementary electrostatic variants (respectively including those described in Table 18), so that VH1 favorably pairs with VL1 and VH2 favorably pairs with VL2. The CH1′ and CL1 include complementary electrostatic variants (including those described in Table 27) and the CH1″ and CL2 include complementary steric variants (including those described in Table 28) so that CH1′ favorably pairs with CL1 and CH1″ favorably pairs with CL2. Panel Q depicts the “1+1 CrossMab” format, with a first Fc comprising a first Fab arm binding a first antigen and a second Fc comprising a second Fab arm binding second antigen. In one embodiment, the 1+1 CrossMab format comprises a first monomer comprising VL1-CH1′-hinge-CH2-CH3, a second monomer comprising VH2-CH1″-hinge-CH2-CH3, a third monomer comprising VH-CL1, and a fourth monomer comprising VL2-CL2. The VL1 pairs with the VH1 to form a binding domain with a first antigen binding specificity; and the VL2 pairs with the VH2 to form a binding domain with a second antigen binding specificity. The CH1″ and CL2 may include electrostatic variants (such as those described in Table 27) or steric variants (such as those described in Table 28) so that the CH1″ favorably pairs with CL2. The CL1 may be kappa or lambda constant light domain, although in preferred embodiments, the CL1 is lambda constant light domain. Panel R depicts the “2+2 Fab₂-scFv₂-Fc” format, with a first Fab-scFv arm and a second Fab-scFv arm, wherein the Fab binds a first antigen and the scFv binds second antigen. The 2+2 Fab₂-scFv-Fc format comprises a first monomer comprising a first heavy chain variable region (VH1) covalently (optionally via a linker) to a single-chain Fv covalently attached (optionally via a linker) to the N-terminus of a first heterodimeric Fc backbone (optionally via a linker), a second monomer comprising the VH1 covalently attached (optionally via a linker) to a single-chain Fv covalently attached (optionally via a linker) to the N-terminus of a second corresponding heterodimeric Fc backbone, and a third monomer comprising a light chain variable region covalently to a light chain constant domain, wherein the light chain variable region is complementary to the VH1. Additional bispecific formats include G) dual scFv, H) one-arm scFv-mAb, I) scFv-mAb, J) bispecific mAb, K) one-arm central-scFv, L) mAb-Fv, M) central-Fv, and N) trident.

FIG. 2 depicts the functional activity of IL23×TL1A bsAbs in a luciferase assay.

FIG. 3 depicts the functional activity of TL1A×IL23 bsAbs in a luciferase assay.

FIG. 4 depicts schematics showing (A) correct Fab pairings and (B) incorrect Fab pairings resulting from introduction of electrostatic variants at K213/K218:D122/E123 in the CH1:CL interface.

FIG. 5 depicts schematics showing (A) correct Fab pairings and (B) incorrect Fab pairings resulting from introduction of steric variants at A141:F118 in the CH1:CL interface.

FIG. 6 depicts schematics showing (A) correct Fab pairings and (B) incorrect Fab pairings resulting from introduction of electrostatic variants at Q39:Q38 in the VH:VL interface.

FIG. 7 depicts schematics showing (A) correct Fab pairings and (B) incorrect Fab pairings resulting from Q39E:Q38K in VH:VL interface and K213E/K218D:D122K/E123K in CH1:CL interface of Fab A, and Q39K:Q38E in VH:VL interface and A141F:F118A interface of Fab.

FIG. 8 depicts A) binding as measured by Biacore at 37° C. of TL1A analyte to TL1A×IL23 bsAb XENP53321, having the TL1A affinity optimized 041[TL1A]H1.33_L1.27 TL1a binding domain as compared to a control TL1A×IL23 bsAb having the parental 041[TL1A]H1L1 TL1A binding domain, and B) binding as measured Biacore at 37° C. of IL23 analyte to TL1A×IL23 bsAb XENP53282, having the IL23 affinity optimized IL23-A[IL23]H1_L1.117 IL23 binding domain compared to a control TL1A×IL23 bsAb having the parental IL23-A[IL23]H1L1 IL23 binding domain. Affinity optimization resulted in candidates with ~10× increase in affinity toward target antigen compared to parental clones.

FIG. 9 depicts the functional activity of XENP53387 compared to various bivalent TL1A and IL23 control mAbs.

FIG. 10 depicts TL1A inhibition by TL1A×IL-23 bispecific antibodies in the 1+1 OrthoFab format, along with controls, incorporating 069 or 111 anti-TL1A binding domains.

FIG. 11 depicts TL1A inhibition by TL1A×RSV bispecific antibody, along with bivalent TL1A mAb control, incorporating 041 to compare monovalent vs. bivalent inhibition activity.

FIG. 12 depicts TL1A inhibition (including IC₅₀ values) for TL1A×IL-23 bispecific antibodies in various bispecific formats.

FIG. 13 depicts IL23 inhibition (including IC₅₀ values) for TL1A×IL-23 bispecific antibodies in various bispecific formats.

FIG. 14 depicts IL23 inhibition by exemplary TL1A×IL23 bispecific antibodies, along with bivalent IL23 mAb controls using a bioluminescent cell-based assay in which the cells are engineered to express luciferase in response to IL23 signaling.

FIG. 15 depicts TL1A inhibition by exemplary TL1A×IL23 bispecific antibodies, along with bivalent TL1A mAb controls using a TL1A-responsive luciferase reporter Jurkat cell line in which luciferase is expressed under the control of an NF-xB response element.

FIG. 16 depicts serum concentration of indicated TL1A×IL23 bispecific antibodies over time in huFcRn mice after intravenous injection of 5 mg/kg.

FIG. 17 depicts serum concentration of indicated TL1A×IL23 bispecific antibodies over time in huFcRn mice after intravenous injection of 5 mg/kg.

### DETAILED DESCRIPTION

### I. Overview

Provided herein are anti-TL1A antibodies, anti-IL23 antibodies, and anti-TL1A×anti-IL23 bispecific antibodies. TL1A and IL23 are both believed to cause inflammatory responses in various diseases associated with IL23 and TL1A (e.g., inflammatory bowel disease, and Crohn's disease). Without being bound by any particular theory of operation, it is believed that in embodiments, the subject anti-TL1A×anti-TL23 bispecific antibodies advantageously inhibit both the TL1A- and TL23-associated inflammatory responses associated with such diseases. Likewise, it is believed that in embodiments, the subject anti-TL1A antibodies inhibit TL1A-associated inflammatory responses associated with such diseases, and the anti-IL23 antibodies inhibit IL23-associated inflammatory responses associated with such diseases.

Accordingly, in one aspect, provided herein are heterodimeric antibodies that bind to two different antigens, e.g., the antibodies are “bispecific,” in that they bind two different target antigens, generally TL1A and TL23 as described below. These heterodimeric antibodies can bind each of the target antigens either monovalently (e.g., there is a single antigen-binding domain such as a variable heavy and variable light domain pair) or bivalently (there are two antigen-binding domains that each independently bind the antigen). In some embodiments, the heterodimeric antibody provided herein includes one TL1A binding domain and one IL23 binding domain (e.g., heterodimeric antibodies in the “1+1 Fab-scFv-Fc,” “OrthoFab,” “CrossMab,” and “1+1 CLC” formats described herein, which are thus bispecific and bivalent). In other embodiments, the heterodimeric antibody provided herein includes one TL1A binding domain and two IL23 binding domains (e.g., heterodimeric antibodies in the “2+1 Fab2-scFv-Fc,” “2+1 mAb-scFv” and “2+1 Fab2-Fc×scFv-Fc” formats described herein, which are thus bispecific but trivalent, as they contain three antigen-binding domains (ABDs)). The heterodimeric antibodies provided herein are based on the use of different monomers that contain amino acid substitutions (i.e., “skew variants”) that “skew” formation of heterodimers over homodimers, as is more fully outlined below. In some embodiments, the heterodimeric antibodies are also coupled with purification variants (e.g., “pI variants”) that allow simple purification of the heterodimers away from the homodimers, as is similarly outlined below. The heterodimeric bispecific antibodies provided generally rely on the use of engineered or variant Fc domains that can self-assemble in production cells to produce heterodimeric proteins, and methods to generate and purify such heterodimeric proteins.

### II. Nomenclature

The naming nomenclature of particular antigen-binding domains (e.g., TL1A and IL23 binding domains) use a “Hx.xx_Ly.yy” type of format, with the numbers being unique identifiers to particular variable chain sequences. Thus, for example, the TL1A binding domain “70416_041[TL1a]_H1L1” (Tables 7-10) includes a variable heavy domain, H1, and a variable light domain L1. In the case that these sequences are used as scFvs, the designation “H1_L”, indicates that the binding domain includes a variable heavy domain “H1” combined with a variable light domain “L1,” and is in VH-linker-VL orientation, from N- to C-terminus. This molecule with the identical sequences of the heavy and light variable domains but in the reverse order (VL-linker-VH orientation, from N- to C-terminus) would be designated “L1_H1.” Similarly, different constructs may “mix and match” the heavy and light chains as will be evident from the sequence listing and the tables.

### III. Definitions

In order that the application may be more completely understood, several definitions are set forth below. Such definitions are meant to encompass grammatical equivalents.

As used herein, “tumor necrosis factor-like cytokine 1A,” “TL1A,” “TL1a,” “TNFSF15,” “vascular endothelial growth inhibitor,” “VEGI,” and “TNF super family member 15,” all refer to a member of tumor necrosis family that is capable of binding to the functional receptor death receptor 3 (DR3) on T cells. Exemplary sequence for human, mouse, and rat TL1A are provided in SEQ ID NOS: 4-6. Such TL1A sequences are useful for the development of TL1A antigen binding domains. TL1A may also be referred to as tumor necrosis factor superfamily 15 (TNFS15) or vascular endothelial growth inhibitor (VEGI). TL1A can activate both the NF-κB and MAPK signaling pathways, and acts as an autocrine factor to induce apoptosis in endothelial cells. TL1A is also found to inhibit endothelial cell proliferation, and thus is believed to function as an angiogenesis inhibitor. In humans, TL1A is encoded by the TNFSF15 gene, and several TNFSF15 SNPs have been found to be associated with inflammatory bowel disease.

As used herein, “interleukin 23,” “IL-23,” “IL23,” all refer to a heterodimeric inflammatory cytokine that is part of the IL-12 family. IL23 is composed of an IL-12B (IL-12p40) subunit and an IL-23 (IL-23p19, also referred to as IL23p19, IL-23A, or TL23A) subunit. Exemplary sequence for human, mouse, and cynomolgus IL23p19 are provided in SEQ ID NOS: 1-3. Such TL23 sequences are useful for the development of IL23 antigen binding domains. IL23 binds to the heterodimeric IL-23 receptor, which comprises an IL-12Rβ1 and IL-23R. IL-23 is secreted by activated dendritic cells, macrophages or monocytes, as well as innate lymphoid cells and TS T cells. IL-23 imbalance and increase is associated with autoimmune diseases and cancer.

By “ablation” herein is meant a decrease or removal of activity. Thus, for example, “ablating FcγR binding” means the Fc region amino acid variant has less than 50% starting binding as compared to an Fc region not containing the specific variant, with more than 70-80-90-95-98% loss of activity being preferred, and in general, with the activity being below the level of detectable binding in a Biacore, SPR or BLI assay. Of particular use in the ablation of FcγR binding are those shown in Table 1, which generally are added to both monomers. Table 1 depicts useful ablation variants that ablate FcγR binding (also referred to as “knockouts” or “KO” variants). In some embodiments, such ablation variants are included in the Fc domain of both monomers of the subject antibody described herein. In other embodiments, the ablation variants are only included on only one variant Fc domain.

TABLE 1
Ablation Variants

| | |
|---|---|
| G236R | L234A/L235A/D265S |
| S239G | L234A/L235A/P329A |
| S239K | L234A/L235A/P329G |
| S239Q | |
| S239R | |
| V266D | |
| S267K | |
| S267R | |
| H268K | |
| E269R | |
| T299R | |
| T299K | |
| K322A | |
| A327G | |
| A327L | |
| A327N | |
| A327Q | |
| L328E | |
| L328R | |
| P329A | |
| P329H | |
| P329K | |
| A330L | |
| A330S/P331S | |
| I332K | |
| I332R | |
| V266D/A327Q | |
| V266D/P329K | |
| S267R/A327Q | |
| S267R/P329K | |
| G236R/L328R | |
| E233P/L234V/L235A/G236_/S239K | |
| E233P/L234V/L235A/G236_/S267K | |
| E233P/L234V/L235A/G236_/S239K/A327G | |
| E233P/L234V/L235A/G236_/S267K/A327G | |
| E233P/L234V/L235A/G236_{—} | |
| S239K/S267K | |
| S267K/P329K | |

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific phagocytic cells that express FcγRs recognize bound antibody on a target cell and subsequently cause phagocytosis of the target cell.

The term “complement-dependent cytotoxicity” or CDC (also called CMC) refers to a mechanism for inducing cell death in which an Fc effector domain(s) of a target-bound antibody activates a series of enzymatic reactions culminating in the formation of holes in the target cell membrane. Typically, antigen-antibody complexes such as those on antibody-coated target cells bind and activate complement component C1q which in turn activates the complement cascade leading to target cell death. Activation of complement may also result in deposition of complement components on the target cell surface that facilitate ADCC by binding complement receptors (e.g., CR3) on leukocytes.

As used herein, the term “antibody” is used to refer to intact antibodies, antigen-binding fragments thereof, or bispecific and multispecific antibodies. Antibodies provided herein can take on a number of formats as described herein, including intact antibodies as well as antibody derivatives, fragments and mimetics, described herein. Typically, fragments compete with the intact antibody from which they were derived for specific binding to the target and include one or more fragments of an intact antibody that retain the ability to specifically bind to a given antigen. Examples of antigen-binding fragments of antibodies include separate heavy chains, light chains, Fab, Fab′, F(ab′)2, F(ab)c, Dabs, nanobodies, and scFvs. Fragments can be produced by recombinant DNA techniques, or by enzymatic or chemical separation of intact immunoglobulins.

A “monovalent antibody” comprises one antigen-binding site per molecule (e.g., IgG or Fab). In some instances, a monovalent antibody can have more than one antigen-binding sites, but the binding sites are from different antigens.

A “bivalent antibody” comprises two antigen-binding sites per molecule (e.g., IgG). In some instances, the two binding sites have the same antigen specificities. However, bivalent antibodies may be bispecific.

A “monospecific antibody” comprises two identical antigen-binding sites per molecule (e.g., IgG) such that the two binding sites bind identical epitope on the antigen. Thus, they compete with each other on binding to one antigen molecule. Most antibodies found in nature are monospecific. In some instances, a monospecific antibody can also be a monovalent antibody (e.g., Fab).

The term “bispecific antibody” as used herein refers to a hybrid antibody having two different binding specificities, e.g., two different heavy/light chain pairs, giving rise to two antigen-binding sites with specificity for different antigens. The term “multispecific antibody” refers to a hybrid antibody having multiple different binding specificities (e.g., multiple antigen-binding sites with specificity for multiple antigens.

Intact antibodies are “Y” shaped tetramers. Each tetramer is typically composed of two identical pairs of polypeptide chains, each pair having one “light chain” monomer (typically having a molecular weight of about 25 kDa) and one “heavy chain” monomer (typically having a molecular weight of about 50-70 kDa).

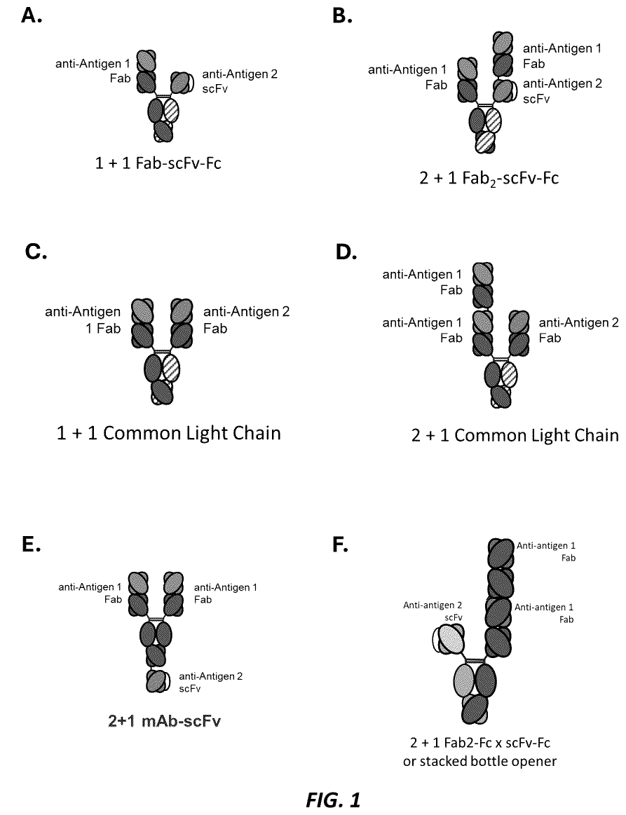

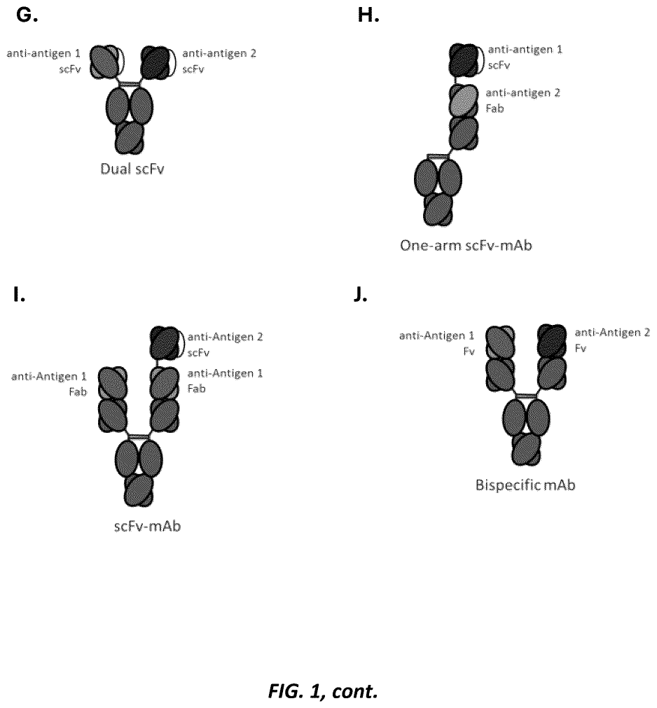

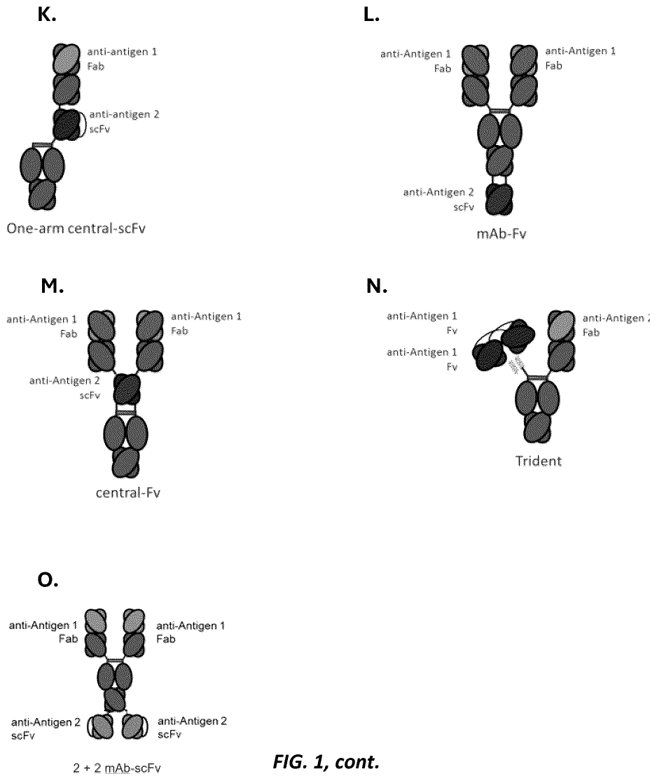

Other useful antibody formats include, but are not limited to, the “1+1 Fab-scFv-Fc,” “1+1 common light chain,” “OrthoFab,” “CrossMab,” and “2+2 mAb-scFv” formats provided herein (see, e.g., FIG. 1). Additional useful antibody formats include, but are not limited to, “2+1 Fab2-scFv-Fc,” “2+1 mAb-scFv,” and “2+1 Fab2-Fc×scFv-Fc,” “2+1 common light chain,” “mAb-Fv,” “mAb-scFv,” “central-Fv,” “one-armed scFv-mAb,” “scFv-mAb,” “dual scFv,” and “trident” format antibodies (FIG. 1). See also, US20180127501A1, which is incorporated by reference herein, particularly in pertinent part relating to antibody formats (see, e.g., FIG. 2 of US20180127501A1).

Antibody heavy chains typically include a variable heavy (VH) domain, which includes vhCDR1-3, and an Fc domain, which includes a CH2-CH3 monomer. In some embodiments, antibody heavy chains include a hinge and CH1 domain. Intact antibody heavy chains are monomers that are organized, from N- to C-terminus: VH-CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, IgG3, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index. “Hinge” refers to positions 216-230 according to the EU index. “CH2” refers to positions 231-340 according to the EU index, and “CH3” refers to positions 341-447 according to the EU index. As shown in Table 2, the exact numbering and placement of the heavy chain domains can be different among different numbering systems. As shown herein and described below, the pI variants can be in one or more of the CH regions, as well as the hinge region, discussed below.

It should be noted that IgG1 has different allotypes with polymorphisms at 356 (D or E) and 358 (L or M). The sequences depicted herein use the 356E/358M allotype, however the other allotype is included herein. That is, any sequence inclusive of an IgG1 Fc domain included herein can have 356D/358L replacing the 356E/358M allotype. It should be understood that therapeutic antibodies can also comprise hybrids of isotypes and/or subclasses. For example, as shown in US Publication 2009/0163699, incorporated by reference, the present antibodies, in some embodiments, include human IgG1/G2 hybrids. Human constant regions show allotypic variation and isoallotypic variation between different individuals: the constant regions can differ in different individuals at one or more polymorphic positions. Isoallotypes differ from allotypes in that sera recognizing an isoallotype bind to a non-polymorphic region of one or more other isotypes.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (C72 and C73), and optionally all or a portion of the hinge region between CH1 (C71) and CH2 (C72). Thus, in some cases, the Fc domain includes, from N- to C-terminal, CH2-CH3 and hinge-CH2-CH3. In some embodiments, the Fc domain is that from IgG1, IgG2, IgG3 or IgG4, with IgG1 hinge-CH2-CH3 and IgG4 hinge-CH2-CH3 finding particular use in many embodiments. Additionally, in the case of human IgG1 Fc domains, the hinge may include a C220S amino acid substitution. Furthermore, in the case of human IgG4 Fc domains, the hinge may include a S228P amino acid substitution. Although the boundaries of the Fc region may vary, the human IgG heavy chain Fc region is usually defined to include residues E216, C226, or A231 to its carboxyl-terminal, wherein the numbering is according to the EU index. In some embodiments, as is more fully described below, amino acid modifications are made to the Fc region, for example to alter binding to one or more FcγR or to the FcRn.

By “heavy chain constant region” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

Another type of domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus, for IgG the antibody hinge is herein defined to include positions 216 (E216 in IgG1) to 230 (P230 in IgG1), wherein the numbering is according to the EU index. In some cases, a “hinge fragment” is used, which contains fewer amino acids at either or both of the N- and C-termini of the hinge domain. As noted herein, pI variants can be made in the hinge region as well. Many of the antibodies herein have at least one the cysteines at position 220 according to EU numbering (hinge region) replaced by a serine. Generally, this modification is on the “scFv monomer” side (when 1+1 or 2+1 formats are used) for most of the sequences depicted herein, although it can also be on the “Fab monomer” side, or both, to reduce disulfide formation. Specifically included within the sequences herein are one or both of these cysteines replaced (C220S).

The exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 2

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (VL), which includes light chain CDRs vlCDR1-3, and a constant light chain region (often referred to as CL or CK). The antibody light chain is typically organized from N- to C-terminus: VL-CL.

By “antigen-binding domain” or “ABD” herein is meant a set of six Complementary Determining Regions (CDRs) that, when present as part of a polypeptide sequence, specifically binds a target antigen (e.g., TL1A or IL23) as discussed herein. These CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 variable heavy CDRs and vlCDR1, vlCDR2 and vlCDR3 vhCDR3 variable light CDRs. The CDRs are present in the variable heavy domain (vhCDR1-3) and variable light domain (vlCDR1-3). The variable heavy domain and variable light domain from an Fv region.

The present invention provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lefranc et al., Dev. Comp. Immunol. 27(1):55-77 (2003):

TABLE 3

| | \hat{ }Kabat + | | | | | | |
|---|---|---|---|---|---|---|---|
| | Chothia | *IMGT | \hat{ }Kabat | \hat{ }AbM | \hat{ }Chothia | \hat{ }Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| VICDR1 | 24-34 | 27-38 | 24-34 | 24-34 | 24-34 | 30-36 | 27-38 |
| VICDR2 | 50-56 | 56-65 | 50-56 | 50-56 | 50-56 | 46-55 | 56-62 |
| VICDR3 | 89-97 | 105-117 | 89-97 | 89-97 | 89-97 | 89-96 | 97-105 |

*CDR boundaries using IMGT numbering
\hat{ }CDR boundaries using Kabat numbering

Throughout the present specification, the Kabat numbering system is generally used when referring to a residue in the variable domain (approximately, residues 1-107 of the light chain variable region and residues 1-113 of the heavy chain variable region) and the light chain constant region, and the EU numbering system for the heavy chain constant region (CH1, hinge, and Fc regions) (e.g., Kabat et al., supra (1991)). The numbering used in the sequence listing and tables for the identification of the CDRs is Kabat, though other numbering systems can be used as shown, e.g., in Table 3.

The CDRs contribute to the formation of the antigen-binding, or more specifically, epitope binding site of the antigen-binding domains and antibodies. “Epitope” refers to a determinant that interacts with a specific antigen-binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen-binding peptide; in other words, the amino acid residue is within the footprint of the specifically antigen-binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen-binding domains and antibodies herein, but those that compete for binding with the epitopes bound by the enumerated antigen-binding domains.

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2 times, 5 times, 10 times, 20 times or 100 times) inhibits binding of the reference antibody by at least 50% but preferably 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

In some embodiments, the six CDRs of the antigen-binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used. In general, the C-terminus of the scFv domain is attached to the N-terminus of all or part of the hinge in the second monomer.

By “variable region” or “variable domain” as used herein is meant the region of an immunoglobulin that comprises one or more Ig domains substantially encoded by any of the Vx, VW, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen-binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the VH, CH1, VL, and CL immunoglobulin domains, generally on two different polypeptide chains (e.g., VH-CH1 on one chain and VL-CL on the other). Fab may refer to this region in isolation, or this region in the context of a bispecific antibody of the invention. In the context of a Fab, the Fab comprises an Fv region in addition to the CH1 and CL domains.

By “Fv” or “Fv fragment” or “Fv region” as used herein is meant the antibody region that comprises the VL and VH domains. Fv regions can be formatted as both Fabs (as discussed above, generally two different polypeptides that also include the constant regions as outlined above) and single chain Fvs (scFvs), where the vl and vh domains are included in a single peptide, attached generally with a linker as discussed herein.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the tables, the order of the vh and vl domain is indicated in the name, e.g., H.X_L.Y means N- to C-terminal is vh-linker-vl, and L.Y_H.X is vl-linker-vh.

Some embodiments of the subject antibodies provided herein comprise at least one scFv domain, which, while not naturally occurring, generally includes a variable heavy domain and a variable light domain, linked together by a scFv linker. As outlined herein, while the scFv domain is generally from N- to C-terminus oriented as VH-scFv linker-VL, this can be reversed for any of the scFv domains (or those constructed using vh and vl sequences from Fabs), to VL-scFv linker-VH, with optional linkers at one or both ends depending on the format.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233 #, E233( ) or E233del designates a deletion of glutamic acid at position 233. Additionally, EDA233- or EDA233 #designates a deletion of the sequence GluAspAla that begins at position 233.

By “variant protein” or “protein variant,” or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities are determined with antibody sequences maximally aligned by the Kabat numbering convention for light chains and heavy chain variable regions or EU numbering for a heavy chain constant region. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

“Variant” as used herein also refers to particular amino acid modifications that confer particular function (e.g., a “heterodimerization variant,” “pI variant,” “ablation variant,” etc.).

As described below, in some embodiments the parent polypeptide, for example an Fc parent polypeptide, is a human wild-type sequence, such as the heavy constant domain or Fc region from IgG1, IgG2, IgG3 or IgG4, although human sequences with variants can also serve as “parent polypeptides”, for example the IgG1/2 hybrid of US Publication 2006/0134105 can be included. The protein variant sequence herein will preferably possess at least about 80% identity with a parent protein sequence, and most preferably at least about 90% identity, more preferably at least about 95-98-99% identity. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2 or IgG4.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/4345. It is noted that the order in which substitutions are provided is arbitrary, that is to say that, for example, 428L/4345 is the same Fc variant as 4345/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

One or several amino acids at the amino or carboxy terminus of the light and/or heavy chain, such as the C-terminal lysine of the heavy chain, may be missing or derivatized in a proportion or all the molecules. Counter ions may or may not be present to form a pharmaceutically acceptable salt. Water or other solvent may or may not be associated with an antibody (e.g., as a hydrate or solvate). Amino acid substitutions can be made in the constant regions to reduce or increase effector functions such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). For example, there are many known mutations in IgG Fc that increase FcRn binding. Exemplary substitutions include Gln at position 250 and/or Leu at position 428, Ser or Asn at position 434, Tyr at position 252, Thr at position 254, Glu at position 256, and Ala at position 434 (EU numbering). Increased FcRn binding is advantageous in making hybrid proteins compete more strongly with endogenous IgG for binding to FcRn. Numerous mutations are also known for reducing any of ADCC, ADCP or CDC (see, e.g., Winter et al., U.S. Pat. No. 5,624,821; Tso et al., U.S. Pat. No. 5,834,597; and Lazar et al., Proc. Natl. Acad. Sci. USA 103:4005, 2006). For example, substitution of any of amino acid residues at positions 234, 235, 236 and/or 237 reduce affinity for Fcγ receptors, particularly FcγRI receptor (see, e.g., U.S. Pat. No. 6,624,821). Optionally, amino acid residues at positions 234, 236 and/or 237 in human IgG2 are substituted with Ala and at position 235 with Gln or Glu (see, e.g., U.S. Pat. No. 5,624,821). Other substitutions reducing effector functions include Ala at position 268, Gly or Ala at position 297, Leu at position 309, Ala at position 322, Gly at position 327, Ser at position 330, Ser at position 331, Ser at position 238, Ala at position 268, Leu at position 309.

In general, variant Fe domains have at least about 80, 85, 90, 95, 97, 98 or 99 percent identity to the corresponding parental human IgG Fc domain (using the identity algorithms discussed below, with one embodiment utilizing the BLAST algorithm, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor,” “FcγR,” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. The functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CH1, CH2, CH3 or hinge domain).

By “target antigen” as used herein is meant the molecule that is bound specifically by the antigen-binding domain comprising the variable regions of a given antibody.

By “strandedness” in the context of the monomers of the heterodimeric antibodies of the invention herein is meant that, similar to the two strands of DNA that “match,” heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

By “target cell” as used herein is meant a cell that expresses a target antigen.

By “host cell” in the context of producing a bispecific antibody according to the invention herein is meant a cell that contains the exogeneous nucleic acids encoding the components of the bispecific antibody and is capable of expressing the bispecific antibody under suitable conditions. Suitable host cells are discussed below.

By “wild type” or “WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T.F. & Waterman, M.S. (1981) “Comparison Of Biosequences,” Adv. Appl. Math. 2:482 [local homology algorithm]; Needleman, S.B. & Wunsch, CD. (1970) “A General Method Applicable To The Search For Similarities In The Amino Acid Sequence Of Two Proteins,” J. Mol. Biol. 48:443 [homology alignment algorithm], Pearson, W.R. & Lipman, D.J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well. This means that an antibody is typically at least 50% w/w pure of interfering proteins and other contaminants arising from its production or purification but does not exclude the possibility that the antibody is combined with an excess of pharmaceutical acceptable carrier(s) or other vehicle intended to facilitate its use. Sometimes antibodies are at least 60, 70, 80, 90, 95 or 99% w/w pure of interfering proteins and contaminants from production or purification. Often an antibody is the predominant macromolecular species remaining after its purification.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

An antibody specifically binding to a target can also be referred to as an antibody against the target. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not, however, necessarily imply that an antibody with the same two binding sites binds only one target or a bispecific antibody with two different binding sites binds only against targets for these two binding sites.

The term “subject” includes human and other mammalian subjects that receive either prophylactic or therapeutic treatment. Other mammalian subjects include animal models of a human condition (e.g., non-human primate or rodent, such as mouse or rat) and veterinary subjects.

Compositions or methods “comprising” one or more recited elements may include other elements not specifically recited. For example, a composition that comprises an antibody may contain the antibody alone or in combination with other ingredients.

### IV. Anti-TL1A Antibodies, Anti-IL23 Antibodies, and Anti-TL1A×Anti-IL23 Antibodies

In one aspect, provided herein are novel anti-TL1A×anti-IL23 antibodies. In another aspect, provided herein are novel anti-TL1A antibodies. In another aspect, provided herein are novel anti-IL23 antibodies. In another aspect, provided herein are combinations of an anti-TL1A antibody and an IL23 antibody.

The anti-TL1A×anti-IL23 antibodies are multivalent and include at least two antigen-binding domains (ABDs), wherein at least one antigen-binding domain is a TL1A binding domain and at least one antigen-binding domain is an IL23 binding domain (e.g., an IL23p19 binding domain). For example, at least one antigen-binding domain can be a human TL1A binding domain and at least one antigen-binding domain can be a human IL23 binding domain (e.g., a human IL23p19 binding domain). Any suitable TL1A binding domain and IL23 binding domain can be included in the subject anti-TL1A×anti-IL23 antibodies, including, for example, the TL1A binding domains and IL23 binding domains provided herein. Likewise, any suitable TL1A binding domain can be included in the subject anti-TL1A antibodies, including, for example, the TL1A binding domains provided herein. Likewise, any suitable IL23 binding domain can be included in the subject anti-IL23 antibodies, including, for example, the IL23 binding domains provided herein.

The antigen-binding domains provided herein generally include a variable heavy domain (VH) having a VH-CDR1, VH-CDR-2, and VH-CDR-3; and a variable light domain (VL), and a variable light domain (VL) having a VL-CDR1, VL-CDR-2, and VL-CDR-3.

In addition, as discussed above, the numbering used in the sequence listing and tables for the identification of the CDRs is Kabat, however, different numbering can be used, which will change the amino acid sequences of the CDRs as shown in Table 3.

For all of the variable heavy and light domains listed herein, further variants can be made. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 80, 85, 90, 95 or 99% identity to a human germline sequence selected from those listed in FIG. 1 of U.S. Pat. No. 7,657,380, which Figure and Legend is incorporated by reference in its entirety herein. Thus, for example, the identical CDRs as described herein can be combined with different framework sequences from human germline sequences, as long as the framework regions retain at least 80, 85 or 90% identity to a human germline sequence selected from those listed in FIG. 1 of U.S. Pat. No. 7,657,380. Alternatively, the CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.)), as well as having framework region changes, as long as the framework regions retain at least 80, 85, 95 to 99% identity to a human germline sequence selected from those listed in FIG. 1 of U.S. Pat. No. 7,657,380.

Any set of 6 CDRs or VH and VL domains can be in the scFv format or in the Fab format, which is then added to the heavy and light constant domains, where the heavy constant domains comprise variants (including within the CH1 domain as well as the Fc domain).

In addition, in embodiments wherein the subject antibody includes an scFv, the scFv can be in an orientation from N- to C-terminus of VH-scFv linker-VL or VL-scFv linker-VH. In some formats, one or more of the ABDs generally is a Fab that includes a VH domain on one protein chain (generally as a component of a heavy chain) and a VL on another protein chain (generally as a component of a light chain). Exemplary scFv linkers for use in the subject antibodies are depicted in Table 4. Table 4 depicts a number of charged scFv linkers that find use in increasing or decreasing the pI of the subject heterodimeric bispecific antibodies that utilize one or more scFv as a component, as described herein (e.g., anti-TL1A×anti-IL23 bsAbs). The (+H) positive linker finds particular use herein, particularly with anti-TL1A and anti-IL23 VL and VH sequences shown herein. A single prior art scFv linker with a single charge is referenced as “Whitlow,” from Whitlow et al., Protein Engineering 6(8):989-995 (1993). It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc and 2+1 Fab2-scFv-Fc formats).

TABLE 4
Positively Charged scFv Linkers

| Name | Sequence | Length | Charge | |
|---|---|---|---|---|
| Gly-Ser 15 | GGGGSGGGGSGGGGS | 15 | 0 | SEQ ID NO: 7 |
| Whitlow linker | GSTSGSGKPGSGEGSTKG | 18 | +1 | SEQ ID NO: 8 |
| 6paxA_1 (+A) | IRPRAIGGSKPRVA | 14 | +4 | SEQ ID NO: 9 |
| +B | GKGGSGKGGSGKGGS | 15 | +3 | SEQ ID NO: 10 |
| +C | GGKGSGGKGSGGKGS | 15 | +3 | SEQ ID NO: 11 |
| +D | GGGKSGGGKSGGGKS | 15 | +3 | SEQ ID NO: 12 |
| +E | GKGKSGKGKSGKGKS | 15 | +6 | SEQ ID NO: 13 |
| +F | GGGKSGGKGSGKGGS | 15 | +3 | SEQ ID NO: 14 |
| +G | GKPGSGKPGSGKPGS | 15 | +3 | SEQ ID NO: 15 |
| +H | GKPGSGKPGSGKPGSGKPGS | 20 | +4 | SEQ ID NO: 16 |
| +I | GKGKSGKGKSGKGKSGKGKS | 20 | +8 | SEQ ID NO: 17 |

Negatively Charged scFv Linkers

| Name | Sequence | Length | Charge | |
|---|---|---|---|---|
| Gly-Ser 20 | GGGGSGGGGSGGGGSGGGGS | 20 | 0 | SEQ ID NO: 18 |
| 3hsc_2 (-A) | STAGDTHLGGEDFD | 14 | −4 | SEQ ID NO: 19 |
| −B | GEGGSGEGGSGEGGS | 15 | −3 | SEQ ID NO: 20 |
| −C | GGEGSGGEGSGGEGS | 15 | −3 | SEQ ID NO: 21 |
| −D | GGGESGGGESGGGES | 15 | −3 | SEQ ID NO: 22 |
| −E | GEGESGEGESGEGES | 15 | −6 | SEQ ID NO: 23 |
| −F | GGGESGGEGSGEGGS | 15 | −3 | SEQ ID NO: 24 |
| −G | GEGESGEGESGEGESGEGES | 20 | −8 | SEQ ID NO: 25 |

Additional scFv Linkers

| | |
|---|---|
| GGGGSGGGGSGGGGS | SEQ ID NO: 7 |
| GGGGSGGGGSGGGGSGGGGS | SEQ ID NO: 18 |
| GSTSGSGKPGSGEGSTKG | SEQ ID NO: 26 |
| PRGASKSGSASQTGSAPGS | SEQ ID NO: 27 |
| GTAAAGAGAAGGAAAGAAG | SEQ ID NO: 28 |
| GTSGSSGSGSGGSGSGGGG | SEQ ID NO: 29 |
| GKPGSGKPGSGKPGSGKPGS | SEQ ID NO: 16 |

In some embodiments, the anti-TL1A antibody is a monospecific antibody. In some embodiments, the anti-TL1A antibody is a bispecific antibody. In some embodiments, the anti-TL1A antibody is a bivalent antibody. In some embodiments, the anti-TL1A antibody is a trivalent antibody. In some embodiments, the anti-TL1A antibody is a bispecific, bivalent antibody.

In some embodiments, the anti-IL23 antibody is a monospecific antibody. In some embodiments, the anti-IL23 antibody is a bispecific antibody. In some embodiments, the anti-IL23 antibody is a bivalent antibody. In some embodiments, the anti-IL23 antibody is a trivalent antibody. In some embodiments, the anti-IL23 antibody is a bispecific, bivalent antibody.

In some embodiments, the anti-TL1A×anti-IL23 antibody is a bispecific antibody. In some embodiments, the TL1A binding domain is monovalent. In some embodiments, the IL23 binding domain is monovalent. In some embodiments, the TL1A binding domain and the IL23 binding domain are each monovalent. In some embodiments, the anti-TL1A×anti-IL23 antibody is a bivalent antibody. In some embodiments, the anti-TL1A×anti-IL23 antibody is a trivalent antibody. In some embodiments, the anti-TL1A×anti-IL23 antibody is a bispecific, bivalent antibody. In some embodiments, the anti-TL1A×anti-IL23 antibodies include one TL1A binding domain and one IL23 binding domain. In exemplary embodiments, the anti-TL1A×anti-IL23 antibody is a bispecific, trivalent antibody. In some embodiments, the anti-TL1A×anti-IL23 antibodies include one TL1A binding domain and two IL23 binding domains. In some embodiments, the anti-TL1A×anti-IL23 antibodies include one IL23 binding domain and two TL1A binding domains.

The anti-TL1A×anti-IL23 antibodies provided herein can be in any useful format, including, including, for example, canonical immunoglobulin, as well as the “1+1 Fab-scFv-Fc,” “2+2 “mAb-scFv,” “2+1 “mAb-scFv,” “2+1 Fab₂-scFv-Fc,” “2+1 Fab₂-Fc×scFv-Fc,” “1+1 common light chain,” and “2+1 common light chain” formats described herein (FIG. 1). Additional useful formats include, for example, the “OrthoFab” and “CrossMab” formats described herein (FIG. 1). Additional useful formats include, but are not limited to: “mAb-Fv,” “central-Fv,” “one-armed scFv-mAb,” “scFv-mAb,” “dual scFv,” and “trident” formats provided herein (see, e.g., FIG. 1). See also, US20180127501A1, which is incorporated by reference herein, particularly in pertinent part relating to antibody formats (see, e.g., FIG. 2). In some embodiments, the anti-TL1A×anti-IL23 antibodies are heterodimeric bispecific antibodies that include variant Fc domains having any of the heterodimerization skew variants, pI variants and/or ablation variants described herein. See, e.g., Table 5, which shows a particularly useful bispecific antibody platform for the anti-TL1A×anti-IL23 bsAbs of the invention. Although the platform is described in the context of the 1+1 Fab-scFv-Fc format, it can be adapted for use in other bispecific antibody formats.

TABLE 5

| Monomer 1 | Monomer 2 |
|---|---|
| ±Heterodimer skew | ±Heterodimer skew |
| variants S364K/E357Q | variants L368D/K370S |
| ±FcKO | ±FcKO |
| E233P/L234V/L235A/ | E233P/L234V/L235A/ |
| G236_/S267K | G236_/S267K |
| ±M428L/N434S for FcRn | ±M428L/N434S for FcRn |

Note that unless specified herein, the order of the antigen list in the name does not confer structure; that is an anti-IL23×anti-TL1A 1+1 Fab-scFv-Fc antibody can have the scFv bind to TL1A or IL23, although in some cases, the order specifies structure as indicated.

The anti-TL1A×anti-IL23 antibodies provided herein further include different antibody domains. As described herein, the antibodies described herein include different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

As shown herein, there are a number of suitable linkers (for use as either domain linkers or scFv linkers) that can be used to covalently attach the recited domains (e.g., scFvs, Fabs, Fc domains, VH domains, VL domains, etc.), including traditional peptide bonds, generated by recombinant techniques. Exemplary linkers to attach domains of the subject antibody to each other are depicted in Table 6, which depicts a number of exemplary domain linkers. In some embodiments, these linkers find use linking a single-chain Fv to an Fc chain. In some embodiments, these linkers may be combined in any orientation. For example, a GGGGS linker may be combined with a “lower half hinge” linker at the N-terminus or at the C-terminus.

TABLE 6

| Name | Sequence | SEQ ID NO: |
|---|---|---|
| (GGGGS)₁ or GGGGS | GGGGS | SEQ ID NO: 31 |
| (GGGGS)₂ | GGGGSGGGGS | SEQ ID NO: 32 |
| (GGGGS)₃ | GGGGSGGGGSGGGGS | SEQ ID NO: 7 |
| (GGGGS)₄ | GGGGSGGGGSGGGGSGGGGS | SEQ ID NO: 18 |
| (GGGGS)₅ | GGGGSGGGGSGGGGSGGGGSGGGGS | SEQ ID NO: 33 |
| (GGGGS)₆ | GGGGSGGGGSGGGGSGGGGSGGGGSGGGGS | SEQ ID NO: 34 |
| (GGGGS)₇ | GGGGSGGGGSGGGGSGGGGSGGGGSGGGGSGGGGS | SEQ ID NO: 35 |
| (GGGGA)₁ or GGGGA | GGGGA | SEQ ID NO: 36 |
| (GGGGA)₂ | GGGGAGGGGA | SEQ ID NO: 37 |
| (GGGGA)₃ | GGGGAGGGGAGGGGA | SEQ ID NO: 38 |
| (GGGGA)₄ | GGGGAGGGGAGGGGAGGGGA | SEQ ID NO: 39 |
| (GGGGA)₅ | GGGGAGGGGAGGGGAGGGGAGGGGA | SEQ ID NO: 40 |
| (GGGGA)₆ | GGGGAGGGGAGGGGAGGGGAGGGGAGGGGA | SEQ ID NO: 41 |
| (GGGGA)₇ | GGGGAGGGGAGGGGAGGGGAGGGGAGGGGAGGGGA | SEQ ID NO: 42 |
| 30AA-linker | DPALVHQRPAPPGGGGSGGGGSGGGGSGGG | SEQ ID NO: 43 |
| (GKPGS)₁ or GKPGS | GKPGS | SEQ ID NO: 44 |
| (GKPGS)₅ | GKPGSGKPGSGKPGSGKPGSGKPGS | SEQ ID NO: 45 |
| (GKPGS)₆ | GKPGSGKPGSGKPGSGKPGSGKPGSGKPGS | SEQ ID NO: 46 |
| (GGGES)₁ or GGGES | GGGES | SEQ ID NO: 47 |
| “full hinge” | EPKSCDKTHTCPPCP | SEQ ID NO: 48 |
| “lower half hinge” | KTHTCPPCP | SEQ ID NO: 49 |
| “full hinge C220S variant” | EPKSSDKTHTCPPCP | SEQ ID NO: 50 |
| “flex lower half hinge” | GGGGSGGGGSKTHTCPPCP | SEQ ID NO: 51 |
| “charged lower half hinge1” | GKPGSGKPGSKTHTCPPCP | SEQ ID NO: 52 |
| “charged lower half hinge2” | GKPGSKTHTCPPCP | SEQ ID NO: 53 |
| “upper half hinge” | EPKSC | SEQ ID NO: 54 |
| “flex upper half hinge” | EPKSCGGGGSGGGGS | SEQ ID NO: 55 |
| “charged upper half hinge1” | EPKSCGKPGSGKPGS | SEQ ID NO: 56 |
| “charged upper half hinge2” | EPKSCGKPGS | SEQ ID NO: 57 |

In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n, (GGGGS)n, and (GGGS)n, where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example, the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KR), hinge region-derived sequences, and other natural sequences from other proteins.

In some embodiments, the linker is a “domain linker,” used to link any two domains as outlined herein together. For example, in the 2+1 Fab₂-scFv-Fc format, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for example (GS)n, (GSGGS)n, (GGGGS)n, and (GGGS)n, where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness,” as outlined below, charged domain linkers, as used in some embodiments of scFv linkers can be used. Exemplary useful domain linkers are depicted in Table 6.

In some embodiments, the linker is a scFv linker that is used to covalently attach the VH and VL domains as discussed herein. In many cases, the scFv linker is a charged scFv linker, a number of which are shown in Table 4. Accordingly, provided herein are charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen-binding domains are calculated, and one is chosen to make an scFv, and depending on the pI, either positive or negative linkers are chosen. Charged domain linkers can also be used to increase the pI separation of the monomers of the invention as well, and thus those included in Table 6 can be used in any embodiment herein where a linker is utilized.

In some embodiments, wherein an scFv is included in the anti-TL1A×anti-IL23 antibody (e.g., the 1+1 Fab-scFv-Fc format antibody), the scFv includes “staple” In exemplary embodiments wherein, such “stapled” scFvs include: a) a first disulfide bond between a structurally conserved surface exposed VH cysteine and a first scFv linker cysteine; b) a second disulfide bond between a structurally conserved surface exposed VL cysteine and a second scFv linker cysteine; or c) the first disulfide bond between the structurally conserved surface exposed VH cysteine and the first scFv linker cysteine and the second disulfide bond between the structurally considered surface exposed VL cysteine and the second scFv linker cysteine. Methods for making “stapled” scFvs are described in WO 2021/030657, which is incorporated by reference in its entirety, including pertinent parts relating to methods for making “stapled” scFvs, and “stapled” scFv compositions.

Exemplary subject anti-TL1A×anti-IL23 antibodies are depicted, for example, in Tables 44, 26, and 29-34. During the cell culture production of the anti-TL1A antibodies, the anti-IL23 antibodies, and the anti-TL1A×anti-TL23 antibodies provided herein, the C-terminal lysine residue or C-terminal lysine and glycine residues may be cleaved from the heavy chain monomers, thereby leading to variants with C-terminal “clipping.” See, e.g., Jiang et al., Journal of Pharmaceutical Sciences 105:2066-2072 (2016). Thus, in some embodiments provided herein, the anti-TL1A×anti-TL23 antibody is a variant of one of the anti-TL1A×anti-IL23 antibodies that includes a deletion of a C-terminal lysine (-K) or C-terminal lysine and glycine (-GK) residues in one or both Fc domains of an anti-TL1A×anti-IL23 antibody described herein. In some embodiments, the deletion is G446del and/or K447del (EU numbering). In some embodiments, the deletion is K447del (EU numbering). In some embodiments, the deletion is G446del and K447del (EU numbering). Likewise, in some embodiments provided herein, the anti-TL1A antibody is a variant of one of the anti-TL1A antibodies that includes a deletion of a C-terminal lysine (-K) or C-terminal lysine and glycine (-GK) residues in one or both Fc domains of an anti-TL1A antibody described herein. In some embodiments, the deletion is G446del and/or K447del (EU numbering). In some embodiments, the deletion is K447del (EU numbering). In some embodiments, the deletion is G446del and K447del (EU numbering). Likewise, in some embodiments provided herein, the anti-TL23 antibody is a variant of one of the anti-IL23 antibodies that includes a deletion of a C-terminal lysine (-K) or C-terminal lysine and glycine (-GK) residues in one or both Fc domains of an anti-TL23 antibody described herein. In some embodiments, the deletion is G446del and/or K447del (EU numbering). In some embodiments, the deletion is K447del (EU numbering). In some embodiments, the deletion is G446del and K447del (EU numbering).

Likewise, during production of the anti-TL1A antibodies, the anti-IL23 antibodies, and the anti-TL1A×anti-IL23 antibodies, glutamine or glutamate at the N-termini of heavy chain and/or light chain can cyclize spontaneously to pyroglutamate. See, e.g., Liu et al. (2011) J. Biol. Chem. 286(13):11211-11217 and Cao et al. (2022) J. Pharm. Sci. 111(2):335-344. Thus, in some embodiments provided herein, the anti-TL1A×anti-IL23 antibody is a variant of one of the anti-TL1A×anti-IL23 antibodies that includes a modification of an N-terminal glutamine or N-terminal glutamate to pyroglutamate in one or more or all of the heavy and light chains of an anti-TL1A×anti-IL23 antibody described herein. In some embodiments, one or both heavy chains of an anti-TL1A×anti-IL23 antibody described herein can include a deletion of a C-terminal lysine (-K) or C-terminal lysine and glycine (-GK) residues and a modification of an N-terminal glutamine or N-terminal glutamate to pyroglutamate. Likewise, in some embodiments provided herein, the anti-TL1A antibody is a variant of one of the anti-TL1A antibodies that includes a modification of an N-terminal glutamine or N-terminal glutamate to pyroglutamate in one or more or all of the heavy and light chains of an anti-TL1A antibody described herein. In some embodiments, one or both heavy chains of an anti-TL1A antibody described herein can include a deletion of a C-terminal lysine (-K) or C-terminal lysine and glycine (-GK) residues and a modification of an N-terminal glutamine or N-terminal glutamate to pyroglutamate. Likewise, in some embodiments provided herein, the anti-IL23 antibody is a variant of one of the anti-IL23 antibodies that includes a modification of an N-terminal glutamine or N-terminal glutamate to pyroglutamate in one or more or all of the heavy and light chains of an anti-IL23 antibody described herein. In some embodiments, one or both heavy chains of an anti-TL1A×anti-IL23 antibody described herein can include a deletion of a C-terminal lysine (-K) or C-terminal lysine and glycine (-GK) residues and a modification of an N-terminal glutamine or N-terminal glutamate to pyroglutamate.

Aspects of the anti-TL1A antibodies, anti-IL23 antibodies, and anti-TL1A×anti-IL23 antibodies are further described in detail below.

### A. TL1A Binding Domains

The anti-TL1A×anti-IL23 antibodies provided herein include at least one TL1A binding domain. Likewise, the anti-TL1A antibodies provided herein include at least one TLA1 binding domain. Any suitable TL1A binding domain can be included in the anti-TL1A×anti-IL23 antibodies or anti-TL1A antibodies provided herein. For example, the TL1A binding domain can be a human TL1A binding domain.

Suitable TL1A binding domains can comprise a set of 6 CDRs as depicted in the tables and sequences (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363), either as they are underlined or, in the case where a different numbering scheme is used as described herein and as shown in Table 3, as the CDRs that are identified using other alignments within the variable heavy (VH) domain and variable light domain (VL) sequences of those depicted in Tables 7-10 and 23 or in Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363. Suitable TL1AABDs can also include the entire VH and VL sequences as depicted in these sequences and tables, used as scFvs or as Fabs. In some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat.

In one embodiment, the TL1A antigen-binding domain (ABD) includes the 6 CDRs (i.e., vhCDR1-3 and vlCDR1-3) of any of the TL1A binding domains described herein, including the tables and sequences (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23). In some embodiments, the TL1AABD is one of the following TL1AABDs: 70416_041[TL1a]_H1L1, 70417_111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (Tables 7-10). In some embodiments, the TL1AABD is a TL1AABD from Table 44 or 22. Tables 7-10 depict the variable heavy and variable light chain sequences for TL1A binding domains which find use in the invention. As noted herein and is true for every sequence herein containing CDRs, the exact identification of the CDR locations may be slightly different depending on the numbering used as is shown in Table 3, and thus included herein are not only the CDRs that are underlined but also CDRs included within the VH and VL domains using other numbering systems. Furthermore, as for all the sequences in the tables, these VH and VL sequences can be used either in a scFv format or in a Fab format.

TABLE 7
041[TL1a]_H1L1

| | Sequence | SEQ ID NO: |
|---|---|---|
| Variable | QVQLVQSGAEVKKPGASVKVSCKVSGFSVTSYNVHWVRQAPGQGLEWM | 101 |
| Heavy (vh) | GVMWSGGNTDYNSKFQGRVTMTRDTSTNTVYMELSSLRSEDTAVYYCA | |
| Domain | RAGRYYHDSNYYWDFPYWGQGTLVTVSS | |
| vhCDR1 | SYNVH | 102 |
| vhCDR2 | VMWSGGNTDYNSKFQG | 103 |
| vhCDR3 | AGRYYHDSNYYWDFPY | 104 |
| Variable Light | DIQMTQSPSSVSASVGDRVTITCRASEDIYSGLAWYQQKPGKSPKLLIYGA | 105 |
| (vl) Domain | SSLQDGVPSRFSGSGSGTDYTLTISSLQPEDFATYFCQEGLKSPYTFGAGTK | |
| | LEIK | |
| VlCDR1 | RASEDIYSGLA | 106 |
| VlCDR2 | GASSLQD | 107 |
| VlCDR3 | QEGLKSPYT | 108 |

111 [TL1a]_H1L1

| | Sequence | SEQ ID NO: |
|---|---|---|
| Variable | EVQLVESGGGLVQPGGSLRLSCAASGFTFSSFGMHWVRQAPGKGLEWVS | 109 |
| Heavy (vh) | YISSGSTTIYYADSVKGRFTISRDNAKNSLYLQMNSLRAEDTAVYYCARAP | |
| Domain | LLRGAMDYWGQGTLVTVSS | |
| vhCDR1 | SFGMH | 110 |
| vhCDR2 | YISSGSTTIYYADSVKG | 111 |
| vhCDR3 | APLLRGAMDY | 112 |
| Variable Light | DIVMTQSPDSLAVSLGERATINCKASQDVSTAVVWYQQKPGQSPKLLIYW | 113 |
| (vl) Domain | ASTRHTGVPDRFTGSGSGTDYTLTISSLQAEDVAVYHCQQHYSTPYTFGG | |
| | GTKLEIK | |
| VlCDR1 | KASQDVSTAVV | 114 |
| VlCDR2 | WASTRHT | 115 |
| VlCDR3 | QQHYSTPYT | 116 |

TABLE 8
069 [TL1a]_H1L1

| | Sequence | SEQ ID NO: |
|---|---|---|
| Variable | EVQLVESGGGLVQPGGSLKLSCAASGFDIIDDYIHWVRQASGKGLEWVGR | 117 |
| Heavy (vh) | IDPASANTKYVPSVKGRFTISADTSKNTAYLQMNSLKTEDTAVYYCTRKG | |
| Domain | FAYWGQGTLVTVSS | |
| vhCDR1 | DDYIH | 118 |
| vhCDR2 | RIDPASANTKYVPSVKG | 119 |
| vhCDR3 | KGFAY | 120 |
| Variable Light | NIVMTQTPLSLSVTPGQPASISCKASENVGTYVSWYLQKPGQSPQLLIYGT | 121 |
| (vl) Domain | SNRYTGVPDRFTGSGSATDFTLKISRVEAEDVGVYHCGQSYRYPYTFGGG | |
| | TKLEIK | |
| VlCDR1 | KASENVGTYVS | 122 |
| VlCDR2 | GTSNRYT | 123 |
| VlCDR3 | GOSYRYPYT | 124 |

189[TL1a]_H1L1

| | Sequence | SEQ ID NO: |
|---|---|---|
| Variable | QVQLVQSGAEVKKPGASVKVSCKASGYTFTDYEIHWMRQAPGQGLEWM | 125 |
| Heavy (vh) | GGIDPETGGTAYNQKFQGRVTMTADKSISTAYMELSRLRSDDTAVYYCTRF | |
| Domain | LVHHYAMDYWGQGTTVTVSS | |
| vhCDR1 | DYEIH | 126 |
| vhCDR2 | GIDPETGGTAYNQKFQG | 127 |
| vhCDR3 | FLVHHYAMDY | 128 |
| Variable Light | DVVMTQSPLSLPVTLGQPASISCRSSQSLLDSDGKTYLNWLQKRPGQSPRR | 129 |
| (vl) Domain | LIYLVSKLDSGVPDRFTGSGSGTDFTLKISRVEAEDVGVYYCWQGTQLPFT | |
| | FGAGTKLEIK | |
| VlCDR1 | RSSQSLLDSDGKTYLN | 130 |
| VlCDR2 | LVSKLDS | 131 |
| VlCDR3 | WQGTQLPFT | 132 |

092 [TL1a]_H1L1

| | Sequence | SEQ ID NO: |
|---|---|---|
| Variable | QVQLVQSGAEVKKPGASVKVSCKVSGFSLTNYGVSWVRQAPGQGLEWM | 133 |
| Heavy (vh) | GGIWGDGSTDYDSKFQGRVTMTRDTSTSTVYMELSSLRSEDTAVYFCTRY | |
| Domain | SSYTNWFVYWGQGTLVTVSS | |
| vhCDR1 | NYGVS | 134 |
| vhCDR2 | GIWGDGSTDYDSKFQG | 135 |
| vhCDR3 | YSSYTNWFVY | 136 |
| Variable Light | DIQMTQSPSSLSASVGDRVTITCRAREDIYNGLAWYQQKPGKSPKLLIYNA | 137 |
| (vl) Domain | NRLHTGVPSRFSGSGSGTDFTLTISSLQPEDFATYFCQQYYDYPLTFGSGTK | |
| | VEIK | |
| vlCDR1 | RAREDIYNGLA | 138 |
| VlCDR2 | NANRLHT | 139 |
| VlCDR3 | QQYYDYPLT | 140 |

TABLE 9
201 [TL1a]_H1L1

| | Sequence | SEQ ID NO: |
|---|---|---|
| Variable | QVQLVQSGAEVKKPGASVKVSCKASGYTFTDYYMDWVRQAPGQSLEW | 141 |
| Heavy (vh) | MGYIYPHTGRTDYNQKFQGRVTMTVDKSISTAYMELSRLRSDDTAVYFC | |
| Domain | ARDSNYYFDVWGAGTTVTVSS | |
| vhCDR1 | DYYMD | 142 |
| vhCDR2 | YIYPHTGRTDYNQKFQG | 143 |
| vhCDR3 | DSNYYFDV | 144 |
| Variable Light | DIVMTQSPLSLPVTPGEPASISCRASQNVRTAVAWYLQKPGQSPQALIYLA | 145 |
| (vl) Domain | SNRHTGVPDRFTGSGSGTDFTLKISRVEAEDVGVYFCLQHWNYPYTFGGG | |
| | TKLEIK | |
| VlCDR1 | RASQNVRTAVA | 146 |
| VlCDR2 | LASNRHT | 147 |
| VlCDR3 | LQHWNYPYT | 148 |

073 [TL1a]_H1L1

| | Sequence | SEQ ID NO: |
|---|---|---|
| Variable | QVQLVQSGAEVKKPGASVKVSCKVSGFSLNTYGVIWVRQAPGQGLEWM | 149 |
| Heavy (vh) | GVIWANGNTNYNSKLQGRVTMTRDTSTSTVYMELRSLRSDDTAVYFCAR | |
| Domain | ARYYYSGVLDYWGQGTLVTVSS | |
| vhCDR1 | TYGVI | 150 |
| vhCDR2 | VIWANGNTNYNSKLQG | 151 |
| vhCDR3 | ARYYYSGVLDY | 152 |
| Variable Light | DVVLTQSPLSLPVTLGQPASISCRSSQSLVHSDGNTYLDWYQQRPGQSPRL | 153 |
| (vl) Domain | LIYKVSNRFSGVPDRFIGSGSGSDFTLKISRVEAEDVGVYYCFQATHDPWAF | |
| | GGGTKVEIK | |
| VlCDR1 | RSSQSLVHSDGNTYLD | 154 |
| VlCDR2 | KVSNRFS | 155 |
| VlCDR3 | FQATHDPWA | 156 |

074 [TL1a]_H1L1

| | Sequence | SEQ ID NO: |
|---|---|---|
| Variable | QVQLVQSGAEVKKPGASVKVSCKASDFNIEDDYIHWVRQAPGQGLEWMG | 157 |
| Heavy (vh) | RIEPAGGNTKYAPKFQGRVTMTADTSINTAYMELSRLRSDDTAVYYCARKG | |
| Domain | FAYWGQGTLVTVSS | |
| vhCDR1 | DDYIH | 118 |
| vhCDR2 | RIEPAGGNTKYAPKFQG | 158 |
| vhCDR3 | KGFAY | 120 |
| Variable Light | KIVMTQSPDSLAVSLGERATINCKASENVGTYVSWYQQKPGQSPKLLIYGT | 159 |
| (vl) Domain | SNRYTGVPDRFTGSGSATDFTLTISSLQAEDVAVYHCGQSYSYPYTFGGGT | |
| | KLEIK | |
| VlCDR1 | KASENVGTYVS | 122 |
| VlCDR2 | GTSNRYT | 123 |
| VlCDR3 | GQSYSYPYT | 160 |

TABLE 10
094 [TL1a]_H1L1

| | Sequence | SEQ ID NO: |
|---|---|---|
| Variable | EVQLVESGGGLVKPGGSLRLSCAVSGFSLTSHSVSWVRQAPGKGPEWVG | 161 |
| Heavy (vh) | RMWYDGDTVYNSPVKGRFTISRDTSKNTLYLQMNSLKTEDTAVYYCTRN | |
| Domain | SRYYDVSYHRFVMDAWGQGTTVTVSS | |
| vhCDR1 | SHSVS | 162 |
| vhCDR2 | RMWYDGDTVYNSPVKG | 163 |
| vhCDR3 | NSRYYDVSYHRFVMDA | 164 |
| Variable Light | DTVLTQSPSSLSASVGDRVTITCRASESVSTGMHWYQQKPGKQPKLLIYG | 165 |
| (vl) Domain | ASNLESGVPARFSGSGSGTDFTLTISSLQPEDFATYFCQQSWSDPWTFGGG | |
| | TKVEIK | |
| VlCDR1 | RASESVSTGMH | 166 |
| VlCDR2 | GASNLES | 167 |
| VlCDR3 | QQSWSDPWT | 168 |

292 [TL1a]_H1L1

| | Sequence | SEQ ID NO: |
|---|---|---|
| Variable | QVQLVQSGAEVKKPGASVKVSCKASGYKFTDHYMDWVRQAPGQSLEW | 169 |
| Heavy (vh) | MGYIHPHTGRTSYNQKFQGRVTMTVDRSTNTAYMELSSLRSEDTAVYYCA | |
| Domain | RDSNYVNAVDYWGQGTLVTVSS | |
| vhCDR1 | DHYMD | 170 |
| vhCDR2 | YIHPHTGRTSYNQKFQG | 171 |
| vhCDR3 | DSNYVNAVDY | 172 |
| Variable Light | QVVLTQSPGTLSLSPGERATLSCRASSSVSSSYLHWYQQKPGQSPRLLIYST | 173 |
| (vl) Domain | SNLASGVPSRFSGSGSGTDYTLTISRLEPEDFAVYYCQQYDSSPSTFGSGTK | |
| | LEIK | |
| VlCDR1 | RASSSVSSSYLH | 174 |
| vlCDR2 | STSNLAS | 175 |
| VlCDR3 | QQYDSSPST | 176 |

In addition to the parental CDR sets disclosed in the tables and sequences that form the TL1A ABDs (see, e.g., Tables 44, 7-10, 22, 23, 26, 29-34, and 48, and VH sequences in SEQ TD NOS: 1427-1823 and 2316-2330 and VL sequences in SEQ TD NOS: 1824-1923, and VH/VL pairs set forth in SEQ ID NOS: 2331-2363, or see, e.g., Tables 7-10 and 23), provided herein are variant TL1A ABDs having CDRs that include at least one modification of the TL1A ABD CDRs disclosed herein (see, e.g., Tables 44, 7-10, 22, 23, 26, 29-34, and 48, and VH sequences in SEQ ID NOS: 1427-1823 and 2316-2330 and VL sequences in SEQ TD NOS: 1824-1923, and VH/VL pairs set forth in SEQ TD NOS: 2331-2363, or see, e.g., Tables 7-10 and 23). In one embodiment, the TL1A ABD includes a set of 6 CDRs with 1, 2, 3, 4, 5, 6, 7, 8, 9, 10 amino acid modifications as compared to the 6 CDRs of a TL1AABD as described herein, including the tables and sequences (see, e.g., Tables 44, 7-10, 22, 23, 26, 29-34, and 48, and VH sequences in SEQ TD NOS: 1427-1823 and 2316-2330 and VL sequences in SEQ ID NOS: 1824-1923, and VH/VL pairs set forth in SEQ TD NOS: 2331-2363, or see, e.g., Tables 7-10 and 23). That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In exemplary embodiments, the TL1AABD includes a set of 6 CDRs with 1, 2, 3, 4, 5, 6, 7, 8, 9, 10 amino acid modifications as compared to the 6 CDRs of one of the following TL1AABDs: 70416_041[TL1a]_H1L1, 70417_111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (Tables 7-10). In exemplary embodiments, the TL1AABD includes a set of 6 CDRs with 1, 2, 3, 4, 5, 6, 7, 8, 9, 10 amino acid modifications as compared to the 6 CDRs of a TL1AABD from Table 44 or 22. In certain embodiments, the TL1AABD is capable of binding TL1A antigen, as measured by at least one of a Biacore, surface plasmon resonance (SPR) and/or BLI (biolayer interferometry, e.g., Octet assay) assay, with the latter finding particular use in many embodiments. In particular embodiments, the TL1A ABD is capable of binding human TL1A antigen (see SEQ ID NO: 4). In some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat.

In some embodiments, the TL1AABD of the subject anti-TL1A antibody or anti-TL1A×anti-IL23 antibody includes 6 CDRs that are at least 90, 95, 97, 98, or 99% identical to the 6 CDRs of a TL1AABD as described herein, including the tables and sequences (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23). In exemplary embodiments, the TL1AABD includes 6 CDRs that are at least 90, 95, 97, 98, or 99% identical to the 6 CDRs of one of the following TL1AABDs: 70416_041[TL1a]_H1L1, 70417111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (Tables 7-10). In exemplary embodiments, the TL1AABD includes 6 CDRs that are at least 90, 95, 97, 98, or 99% identical to the 6 CDRs of a TL1AABD in Table 44 or 22. In certain embodiments, the TL1A ABD is capable of binding TL1A antigen, as measured by at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4). In some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat.

In another exemplary embodiment, the TL1AABD of the subject anti-TL1a×anti-IL23 antibody includes the variable heavy (VH) domain and variable light (VL) domain of any one of the TL1AABDs described herein, including the tables and sequences (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363 or see, e.g., Tables 7-10 and 23). In exemplary embodiments, the TL1AABD is one of the following TL1AABDs: 70416_041[TL1a]_H1L1, 70417_111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (Tables 7-10). In exemplary embodiments, the TL1AABD is a TL1AABD from Table 44 or Table 22.

In some embodiments, the anti-TL1A×anti-IL23 antibody includes a TL1A ABD that includes a variable heavy domain and/or a variable light domain that are variants of a TL1AABD VH and VL domain disclosed herein (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23). In one embodiment, the variant VH domain and/or VL domain has from 1, 2, 3, 4, 5, 6, 7, 8, 9 or 10 amino acid changes from a VH and/or VL domain of a TL1AABD described herein, including the tables and sequences (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23). In some embodiments, the variant VH domain and/or VL domain includes 1, 2, 3, 4, 5, 6, 7, 8, 9 or 10 amino acid changes in a framework region (e.g., FR1, FR2, FR3, or FR4) from a VH and/or VL domain of a TL1AABD described herein, including the tables and sequences (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23). In some embodiments, the variant VH and/or VL domain does not have any changes in the CDRs from a VH and/or VL domain of a TL1A ABD described herein but has changes in a framework region. In some embodiments, the variant VH and/or VL domain includes 1, 2, 3, 4, 5, 6, 7, 8, 9 or 10 amino acid changes in a framework region (e.g., FR1, FR2, FR3, or FR4) from a VH and/or VL domain of a TL1A ABD described herein, wherein the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In exemplary embodiments, the variant VH domain and/or VL domain has from 1, 2, 3, 4, 5, 6, 7, 8, 9 or 10 amino acid changes from a VH and/or VL domain of one of the following TL1AABDs: 70416_041[TL1a]_H1L1, 70417_111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (Tables 7-10). In exemplary embodiments, the variant VH domain and/or VL domain has from 1, 2, 3, 4, 5, 6, 7, 8, 9 or 10 amino acid changes from a VH and/or VL domain of a TL1AABD in Table 44 or 22. In exemplary embodiments, the variant VH domain and/or VL domain has from 1, 2, 3, 4, 5, 6, 7, 8, 9 or 10 amino acid changes in a framework region (e.g., FR1, FR2, FR3, or FR4) of the VH and/or VL domain of one of the following TL1AABDs: 70416_041[TL1a]_H1L1, 70417_111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (Tables 7-10). In exemplary embodiments, the variant VH domain and/or VL domain has from 1, 2, 3, 4, 5, 6, 7, 8, 9 or 10 amino acid changes in a framework region (e.g., FR1, FR2, FR3, or FR4) of the VH and/or VL domain of a TL1AABD in Table 44 or 22. In certain embodiments, the TL1A ABD is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1AABD is capable of binding human TL1A antigen (see SEQ ID NO: 4). In some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat.

In one embodiment, the variant VH and/or VL domain is at least 90, 95, 97, 98 or 99% identical to the VH and/or VL of a TL1AABD as described herein, including the tables and sequences (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23). In exemplary embodiments, the variant VH and/or VL domain is at least 90, 95, 97, 98 or 99% identical to the VH and/or VL of one of the following TL1AABDs: 70416_041[TL1a]_H1L1, 70417_111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (Tables 7-10). In exemplary embodiments, the variant VH and/or VL domain is at least 90, 95, 97, 98 or 99% identical to the VH and/or VL of a TL1AABD in Table 44 or 22. In some embodiments, the variant VH and/or VL domain does not have any changes in the CDRs from a VH and/or VL domain of a TL1AABD described herein but has changes in a framework region. In some embodiments, the variant VH and/or VL domain includes amino acid changes in a framework region (e.g., FR1, FR2, FR3, or FR4) from a VH and/or VL domain of a TL1AABD described herein, wherein the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In certain embodiments, the TL1A ABD is capable of binding to TL1A, as measured by at least one of a Biacore, surface plasmon resonance (SPR) and/or BLI (biolayer interferometry, e.g., Octet assay) assay, with the latter finding particular use in many embodiments. In particular embodiments, the TL1AABD is capable of binding human TL1A antigen (see SEQ ID NO: 4). In some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat.

In some embodiments, a TL1A binding domain comprises a variable heavy domain comprising a vhCDR1, a vhCDR2, and a vhCDR3 contained within a variable heavy domain comprising the amino acid sequence of any one of SEQ ID NOS: 2241, 2253, 2259, 1674, and 1200, and a variable light domain comprising a vlCDR1, a vlCDR2, and a vlCDR3 contained within a variable light domain comprising the amino acid sequence of any one of SEQ ID NOS: 2243 and 1204. In some embodiments, a TL1A binding domain comprises a variable heavy domain comprising a vhCDR1, a vhCDR2, and a vhCDR3 contained within a variable heavy domain comprising the amino acid sequence of SEQ ID NO: 2241, and a variable light domain comprising a vlCDR1, a vlCDR2, and a vlCDR3 contained within a variable light domain comprising the amino acid sequence of SEQ ID NO: 2243. In some embodiments, a TL1A binding domain comprises a variable heavy domain comprising a vhCDR1, a vhCDR2, and a vhCDR3 contained within a variable heavy domain comprising the amino acid sequence of SEQ ID NO: 2253, and a variable light domain comprising a vlCDR1, a vlCDR2, and a vlCDR3 contained within a variable light domain comprising the amino acid sequence of SEQ ID NO: 2243. In some embodiments, a TL1A binding domain comprises a variable heavy domain comprising a vhCDR1, a vhCDR2, and a vhCDR3 contained within a variable heavy domain comprising the amino acid sequence of SEQ ID NO: 2259, and a variable light domain comprising a vlCDR1, a vlCDR2, and a vlCDR3 contained within a variable light domain comprising the amino acid sequence of SEQ ID NO: 2243. In some embodiments, a TL1A binding domain comprises a variable heavy domain comprising a vhCDR1, a vhCDR2, and a vhCDR3 contained within a variable heavy domain comprising the amino acid sequence of SEQ ID NO: 1674, and a variable light domain comprising a vlCDR1, a vlCDR2, and a vlCDR3 contained within a variable light domain comprising the amino acid sequence of SEQ ID NO: 1204. In some embodiments, a TL1A binding domain comprises a variable heavy domain comprising a vhCDR1, a vhCDR2, and a vhCDR3 contained within a variable heavy domain comprising the amino acid sequence of SEQ ID NO: 1200, and a variable light domain comprising a vlCDR1, a vlCDR2, and a vlCDR3 contained within a variable light domain comprising the amino acid sequence of SEQ ID NO: 1204. In some embodiments, a TL1A binding domain comprises a variable heavy domain comprising a vhCDR1, a vhCDR2, and a vhCDR3 contained within a variable heavy domain comprising the amino acid sequence of any one of SEQ ID NOS: 1674, 1200, 1661, and 2323, and a variable light domain comprising a vlCDR1, a vlCDR2, and a vlCDR3 contained within a variable light domain comprising the amino acid sequence of SEQ ID NO: 1204. In some embodiments, a TL1A binding domain comprises a variable heavy domain comprising a vhCDR1, a vhCDR2, and a vhCDR3 contained within a variable heavy domain comprising the amino acid sequence of SEQ ID NO: 1200, and a variable light domain comprising a vlCDR1, a vlCDR2, and a vlCDR3 contained within a variable light domain comprising the amino acid sequence of SEQ ID NO: 1204. In some embodiments, a TL1A binding domain comprises a variable heavy domain comprising a vhCDR1, a vhCDR2, and a vhCDR3 contained within a variable heavy domain comprising the amino acid sequence of SEQ ID NO: 1661, and a variable light domain comprising a vlCDR1, a vlCDR2, and a vlCDR3 contained within a variable light domain comprising the amino acid sequence of SEQ ID NO: 1204. In some embodiments, a TL1A binding domain comprises a variable heavy domain comprising a vhCDR1, a vhCDR2, and a vhCDR3 contained within any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some embodiments, a TL1A binding domain comprises a variable heavy domain comprising a vhCDR1, a vhCDR2, and a vhCDR3 contained within a variable heavy domain comprising a vhCDR1, a vhCDR2, and a vhCDR3 contained within a variable heavy domain comprising the amino acid sequence of any one of SEQ ID NOS: 1427-1823 and 2316-2330, and a variable light domain comprising a vlCDR1, a vlCDR2, and a vlCDR3 contained within a variable light domain comprising the amino acid sequence of any one of SEQ ID NOS: 1824-1923. In some embodiments, a TL1A binding domain comprises a variable heavy domain comprising a vhCDR1, a vhCDR2, and a vhCDR3 contained within a variable heavy domain comprising the amino acid sequence of SEQ ID NO: 2323, and a variable light domain comprising a vlCDR1, a vlCDR2, and a vlCDR3 contained within a variable light domain comprising the amino acid sequence of SEQ ID NO: 1204. In some embodiments, the CDRs are as defined by Kabat. In other embodiments, the CDRs are as defined by Chothia, Kabat+Chothia, AbM, contact, or IMGT. In some embodiments, a TL1A binding domain comprises a vhCDR1 comprising the amino acid sequence of SEQ ID NO: 102 or 1201, a vhCDR2 comprising the amino acid sequence of SEQ ID NO: 1924 or 1202, a vhCDR3 comprising the amino acid sequence of SEQ ID NO: 1203 or 104, a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some embodiments, a TL1A binding domain comprises a vhCDR1 comprising the amino acid sequence of SEQ ID NO: 102, a vhCDR2 comprising the amino acid sequence of SEQ ID NO: 1924, a vhCDR3 comprising the amino acid sequence of SEQ ID NO: 1203, a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some embodiments, a TL1A binding domain comprises a vhCDR1 comprising the amino acid sequence of SEQ ID NO: 1201, a vhCDR2 comprising the amino acid sequence of SEQ ID NO: 1202, a vhCDR3 comprising the amino acid sequence of SEQ ID NO: 1203, a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some embodiments, a TL1A binding domain comprises a vhCDR1 comprising the amino acid sequence of SEQ ID NO: 102, a vhCDR2 comprising the amino acid sequence of SEQ ID NO: 1924, a vhCDR3 comprising the amino acid sequence of SEQ ID NO: 104, a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some embodiments, a TL1A binding domain comprises a vhCDR1 comprising the amino acid sequence of SEQ ID NO: 102, a vhCDR2 comprising the amino acid sequence of SEQ ID NO: 1926, a vhCDR3 comprising the amino acid sequence of SEQ ID NO: 104, a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some embodiments, a TL1A binding domain comprises a set of 6 CDRs with 1, 2, 3, 4, 5, 6, 7, 8, 9, 10 amino acid modifications as compared to the 6 CDRs of any of the above TL1A binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, a TL1A binding domain comprises a set of 6 CDRs that are at least 90, 95, 97, 98 or 99% identical to the 6 CDRs of any of the above TL1A binding domains. In some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4). In particular embodiments, the antibody specifically binds human TL1A with a K_{D} of less than about 6.5E-11 M, less than about 6.4 E-11 M, or less than about 6.3E-11 as measured by surface plasmon resonance at 25° C., optionally wherein the antibody specifically binds human TL1A with a K_{D} of less than about 3E-11 M, less than about 2.9E-11 M, less than about 2.8E-11 M, or less than about 2.7E-11 M. In some embodiments, an antibody disclosed herein specifically binds human TL1A with a K_{D} of between about 2E-11 M and about 7E-11M, between about 2.5E-11 M and about 7E-11 M, or between about 2.5E-11 M and about 6.5E-11 M as measured by surface plasmon resonance or Biacore at 25° C. In some embodiments, an antibody disclosed herein specifically binds human TL1A with a K_{D} of between about 2.5E-11 M and about 3.5E-11 M, between about 2.5E-11 M and about 3E-11 M, or between about 2.5E-11 M and about 2.7E-11 M as measured by surface plasmon resonance or Biacore at 25° C. In particular embodiments, the antibody the antibody inhibits TL1A activity in a TL1A luciferase reporter assay with an IC₅₀ of less than about 4.5 nM. In some embodiments, an antibody disclosed herein inhibits TL1A activity in a TL1A luciferase reporter assay with an IC₅₀ of between about 2.5 nM and about 4.5 nM, between about 3.5 nM and about 5.5 nM, or between about 4 nM and about 5 nM.

In some embodiments, the variable heavy domain comprises the amino acid sequence of any one of SEQ ID NOS: 2241, 2253, 2259, 1674, and 1200, and the variable light domain comprises the amino acid sequence of any one of SEQ ID NOS: 2243 and 1204. In some embodiments, the variable heavy domain comprises the amino acid sequence of SEQ ID NO: 2241, and the variable light domain comprises the amino acid sequence of SEQ ID NO: 2243. In some embodiments, the variable heavy domain comprises the amino acid sequence of SEQ ID NO: 2253, and the variable light domain comprises the amino acid sequence of SEQ ID NO: 2243. In some embodiments, the variable heavy domain comprises the amino acid sequence of SEQ ID NO: 2259, and the variable light domain comprises the amino acid sequence of SEQ ID NO: 2243. In some embodiments, the variable heavy domain comprises the amino acid sequence of SEQ ID NO: 1674, and the variable light domain comprises the amino acid sequence of SEQ ID NO: 1204. In some embodiments, the variable heavy domain comprises the amino acid sequence of SEQ ID NO: 1200, and the variable light domain comprises the amino acid sequence of SEQ ID NO: 1204. In some embodiments, the variable heavy domain comprises the amino acid sequence of any one of SEQ ID NOS: 1427-1823 and 2316-2330, and the variable light domain comprises the amino acid sequence of any one of SEQ ID NOS: 1824-1923. In some embodiments, the variable heavy domain comprises the amino acid sequence of any one of SEQ ID NOS: 1674, 1200, 1661, and 2323, and the variable light domain comprises the amino acid sequence of SEQ ID NO: 1204. In some embodiments, the variable heavy domain comprises the amino acid sequence of SEQ ID NO: 1200, and the variable light domain comprises the amino acid sequence of SEQ ID NO: 1204. In some embodiments, the variable heavy domain comprises the amino acid sequence of SEQ ID NO: 1661, and the variable light domain comprises the amino acid sequence of SEQ ID NO: 1204. In some embodiments, the variable heavy domain comprises the amino acid sequence of SEQ ID NO: 2323, and the variable light domain comprises the amino acid sequence of SEQ ID NO: 1204. In some embodiments, the variable heavy domain and the variable light domain comprise the pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some embodiments, the variable heavy domain and the variable light domain comprise any pair of anti-TL1A variable heavy domain and variable light domain in Table 29 or 33. In some such antibodies, the variable heavy domain or the variable light domain can comprise variants of any of the above in which an N-terminal glutamine or glutamate is replaced with pyroglutamate. In some embodiments, the TL1A binding domain comprises a variant VH domain and/or variant VL domain having 1, 2, 3, 4, 5, 6, 7, 8, 9 or 10 amino acid changes from any of the above VH and VL domains. In some embodiments, the TL1A binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4). In particular embodiments, the antibody specifically binds human TL1A with a K_{D} of less than about 6.5E-11 M, less than about 6.4 E-11 M, or less than about 6.3E-11 as measured by surface plasmon resonance at 25° C., optionally wherein the antibody specifically binds human TL1A with a K_{D} of less than about 3E-11 M, less than about 2.9E-11 M, less than about 2.8E-11 M, or less than about 2.7E-11 M. In some embodiments, an antibody disclosed herein specifically binds human TL1A with a K_{D} of between about 2E-11 M and about 7E-11M, between about 2.5E-11 M and about 7E-11 M, or between about 2.5E-11 M and about 6.5E-11 M as measured by surface plasmon resonance or Biacore at 25° C. In some embodiments, an antibody disclosed herein specifically binds human TL1A with a K_{D} of between about 2.5E-11 M and about 3.5E-11 M, between about 2.5E-11 M and about 3E-11 M, or between about 2.5E-11 M and about 2.7E-11 M as measured by surface plasmon resonance or Biacore at 25° C. In particular embodiments, the antibody the antibody inhibits TL1A activity in a TL1A luciferase reporter assay with an IC₅₀ of less than about 4.5 nM. In some embodiments, an antibody disclosed herein inhibits TL1A activity in a TL1A luciferase reporter assay with an IC₅₀ of between about 2.5 nM and about 4.5 nM, between about 3.5 nM and about 5.5 nM, or between about 4 nM and about 5 nM.

In some embodiments, a TL1A binding domain comprises a variable heavy domain comprising a vhCDR1, a vhCDR2, and a vhCDR3 contained within a variable heavy domain comprising the amino acid sequence of SEQ ID NO: 2241, and a variable light domain comprising a vlCDR1, a vlCDR2, and a vlCDR3 contained within a variable light domain comprising the amino acid sequence of SEQ ID NO: 2243. In some embodiments, the CDRs are as defined by Kabat. In other embodiments, the CDRs are as defined by Chothia, Kabat+Chothia, AbM, contact, or IMGT. In some embodiments, the variable heavy domain comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, and (ii) the variable light domain comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some embodiments, a TL1A binding domain comprises a set of 6 CDRs with 1, 2, 3, 4, 5, 6, 7, 8, 9, 10 amino acid modifications as compared to the 6 CDRs of any of the above TL1A binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, a TL1A binding domain comprises a set of 6 CDRs that are at least 90, 95, 97, 98 or 99% identical to the 6 CDRs of any of the above TL1A binding domains. In some embodiments, the variable heavy domain comprises the amino acid sequence of SEQ ID NO: 2241, and the variable light domain comprises the amino acid sequence of SEQ ID NO: 2243. In some such antibodies, the variable heavy domain or the variable light domain can comprise variants of any of the above in which an N-terminal glutamine or glutamate is replaced with pyroglutamate. In some embodiments, the TL1A binding domain comprises a variant VH domain and/or variant VL domain having 1, 2, 3, 4, 5, 6, 7, 8, 9 or 10 amino acid changes from any of the above VH and VL domains. In some embodiments, the TL1A binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4).

In some embodiments, a TL1A binding domain comprises a variable heavy domain comprising a vhCDR1, a vhCDR2, and a vhCDR3 contained within a variable heavy domain comprising the amino acid sequence of SEQ ID NO: 2253, and a variable light domain comprising a vlCDR1, a vlCDR2, and a vlCDR3 contained within a variable light domain comprising the amino acid sequence of SEQ ID NO: 2243. In some embodiments, the CDRs are as defined by Kabat. In other embodiments, the CDRs are as defined by Chothia, Kabat+Chothia, AbM, contact, or IMGT. In some embodiments, the variable heavy domain comprises a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, and (ii) the variable light domain comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some embodiments, a TL1A binding domain comprises a set of 6 CDRs with 1, 2, 3, 4, 5, 6, 7, 8, 9, 10 amino acid modifications as compared to the 6 CDRs of any of the above TL1A binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, a TL1A binding domain comprises a set of 6 CDRs that are at least 90, 95, 97, 98 or 99% identical to the 6 CDRs of any of the above TL1A binding domains. In some embodiments, the variable heavy domain comprises the amino acid sequence of SEQ ID NO: 2253, and the variable light domain comprises the amino acid sequence of SEQ ID NO: 2243. In some such antibodies, the variable heavy domain or the variable light domain can comprise variants of any of the above in which an N-terminal glutamine or glutamate is replaced with pyroglutamate. In some embodiments, the TL1A binding domain comprises a variant VH domain and/or variant VL domain having 1, 2, 3, 4, 5, 6, 7, 8, 9 or 10 amino acid changes from any of the above VH and VL domains. In some embodiments, the TL1A binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4).

In some embodiments, a TL1A binding domain comprises a variable heavy domain comprising a vhCDR1, a vhCDR2, and a vhCDR3 contained within a variable heavy domain comprising the amino acid sequence of SEQ ID NO: 2259, and a variable light domain comprising a vlCDR1, a vlCDR2, and a vlCDR3 contained within a variable light domain comprising the amino acid sequence of SEQ ID NO: 2243. In some embodiments, the CDRs are as defined by Kabat. In other embodiments, the CDRs are as defined by Chothia, Kabat+Chothia, AbM, contact, or IMGT. In some embodiments, the variable heavy domain comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 104, and (ii) the variable light domain comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some embodiments, a TL1A binding domain comprises a set of 6 CDRs with 1, 2, 3, 4, 5, 6, 7, 8, 9, 10 amino acid modifications as compared to the 6 CDRs of any of the above TL1A binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, a TL1A binding domain comprises a set of 6 CDRs that are at least 90, 95, 97, 98 or 99% identical to the 6 CDRs of any of the above TL1A binding domains. In some embodiments, the variable heavy domain comprises the amino acid sequence of SEQ ID NO: 2259, and the variable light domain comprises the amino acid sequence of SEQ ID NO: 2243. In some such antibodies, the variable heavy domain or the variable light domain can comprise variants of any of the above in which an N-terminal glutamine or glutamate is replaced with pyroglutamate. In some embodiments, the TL1A binding domain comprises a variant VH domain and/or variant VL domain having 1, 2, 3, 4, 5, 6, 7, 8, 9 or 10 amino acid changes from any of the above VH and VL domains. In some embodiments, the TL1A binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4).

In some embodiments, a TL1A binding domain comprises a variable heavy domain comprising a vhCDR1, a vhCDR2, and a vhCDR3 contained within a variable heavy domain comprising the amino acid sequence of SEQ ID NO: 1674, and a variable light domain comprising a vlCDR1, a vlCDR2, and a vlCDR3 contained within a variable light domain comprising the amino acid sequence of SEQ ID NO: 1204. In some embodiments, the CDRs are as defined by Kabat. In other embodiments, the CDRs are as defined by Chothia, Kabat+Chothia, AbM, contact, or IMGT. In some embodiments, the variable heavy domain comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, and (ii) the variable light domain comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some embodiments, a TL1A binding domain comprises a set of 6 CDRs with 1, 2, 3, 4, 5, 6, 7, 8, 9, 10 amino acid modifications as compared to the 6 CDRs of any of the above TL1A binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, a TL1A binding domain comprises a set of 6 CDRs that are at least 90, 95, 97, 98 or 99% identical to the 6 CDRs of any of the above TL1A binding domains. In some embodiments, the variable heavy domain comprises the amino acid sequence of SEQ ID NO: 1674, and the variable light domain comprises the amino acid sequence of SEQ ID NO: 1204. In some such antibodies, the variable heavy domain or the variable light domain can comprise variants of any of the above in which an N-terminal glutamine or glutamate is replaced with pyroglutamate. In some embodiments, the TL1A binding domain comprises a variant VH domain and/or variant VL domain having 1, 2, 3, 4, 5, 6, 7, 8, 9 or 10 amino acid changes from any of the above VH and VL domains. In some embodiments, the TL1A binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4).

In some embodiments, a TL1A binding domain comprises a variable heavy domain comprising a vhCDR1, a vhCDR2, and a vhCDR3 contained within a variable heavy domain comprising the amino acid sequence of SEQ ID NO: 1200, and a variable light domain comprising a vlCDR1, a vlCDR2, and a vlCDR3 contained within a variable light domain comprising the amino acid sequence of SEQ ID NO: 1204. In some embodiments, the CDRs are as defined by Kabat. In other embodiments, the CDRs are as defined by Chothia, Kabat+Chothia, AbM, contact, or IMGT. In some embodiments, the variable heavy domain comprises a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, and (ii) the variable light domain comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108. In some embodiments, a TL1A binding domain comprises a set of 6 CDRs with 1, 2, 3, 4, 5, 6, 7, 8, 9, 10 amino acid modifications as compared to the 6 CDRs of any of the above TL1A binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, a TL1A binding domain comprises a set of 6 CDRs that are at least 90, 95, 97, 98 or 99% identical to the 6 CDRs of any of the above TL1A binding domains. In some embodiments, the variable heavy domain comprises the amino acid sequence of SEQ ID NO: 1200, and the variable light domain comprises the amino acid sequence of SEQ ID NO: 1204. In some such antibodies, the variable heavy domain or the variable light domain can comprise variants of any of the above in which an N-terminal glutamine or glutamate is replaced with pyroglutamate. In some embodiments, the TL1A binding domain comprises a variant VH domain and/or variant VL domain having 1, 2, 3, 4, 5, 6, 7, 8, 9 or 10 amino acid changes from any of the above VH and VL domains. In some embodiments, the TL1A binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4).

In some embodiments, an anti-TL1A antibody or an anti-TL1A×anti-IL23 antibody comprises a heavy chain comprising the amino acid sequence of SEQ ID NO: 2240 or a variant thereof in which the N-terminal glutamine is replaced with pyroglutamate and/or the C-terminal lysine or the C-terminal lysine and glycine are removed, and a light chain comprising the amino acid sequence of SEQ ID NO: 2242. In some embodiments, an anti-TL1A antibody or an anti-TL1A×anti-IL23 antibody comprises a heavy chain comprising the amino acid sequence of SEQ ID NO: 2252 or a variant thereof in which the N-terminal glutamine is replaced with pyroglutamate and/or the C-terminal lysine or the C-terminal lysine and glycine are removed, and a light chain comprising the amino acid sequence of SEQ ID NO: 2242. In some embodiments, an anti-TL1A antibody or an anti-TL1A×anti-IL23 antibody comprises a heavy chain comprising the amino acid sequence of SEQ ID NO: 2258 or a variant thereof in which the N-terminal glutamine is replaced with pyroglutamate and/or the C-terminal lysine or the C-terminal lysine and glycine are removed, and a light chain comprising the amino acid sequence of SEQ ID NO: 2242. In some embodiments, an anti-TL1A antibody or an anti-TL1A×anti-IL23 antibody comprises a heavy chain comprising the amino acid sequence of SEQ ID NO: 2264 or a variant thereof in which the C-terminal lysine or the C-terminal lysine and glycine are removed, and a light chain comprising the amino acid sequence of SEQ ID NO: 2265 or a variant thereof in which the N-terminal glutamine is replaced with pyroglutamate. In some embodiments, an anti-TL1A antibody or an anti-TL1A×anti-IL23 antibody comprises a heavy chain comprising the amino acid sequence of SEQ ID NO: 2264 or a variant thereof in which the C-terminal lysine or the C-terminal lysine and glycine are removed, and a light chain comprising the amino acid sequence of SEQ ID NO: 2269 or a variant thereof in which the N-terminal glutamine is replaced with pyroglutamate. In some embodiments, an anti-TL1A antibody or an anti-TL1A×anti-IL23 antibody comprises an anti-TL1A heavy chain and light chain pair from XENP53412, XENP53413, XENP53369, XENP53375, XENP53370, XENP53371, XENP53376, XENP53377, XENP53378, XENP53379, XENP53380, or XENP53414, or heavy chain variants thereof in which the N-terminal glutamine is replaced with pyroglutamate and/or the C-terminal lysine or the C-terminal lysine and glycine are removed, or comprises an anti-TL1A heavy chain and light chain pair from XENP53415 or XENP53387 or heavy chain variants thereof in which the C-terminal lysine or the C-terminal lysine and glycine are removed and/or light chain variants thereof in which the N-terminal glutamine is replaced with pyroglutamate. In some embodiments, an anti-TL1A antibody or an anti-TL1A×anti-IL23 antibody comprises a heavy chain and/or a light chain having 1, 2, 3, 4, 5, 6, 7, 8, 9 or 10 amino acid changes from any of the above heavy chains and light chains. In some embodiments, an anti-TL1A antibody or an anti-TL1A×anti-IL23 antibody comprises a heavy chain and/or a light chain at least 90%, 95%, 97%, 98%, or 99% identical to any of the above heavy chains and light chains. In some embodiments, the VH sequence includes 53Y (i.e., a Y at position 53) and/or 100V (i.e., a V at position 100) as numbered by Kabat. In some embodiments, the VH sequence includes 53Y and 100V In some embodiments, the VL sequence includes 120Q (i.e., a Q at position 120) as numbered by Kabat. In some embodiments, the amino acid changes are in a constant region but not in a variable region. In some embodiments, the amino acid changes are in a constant region or in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4).

### B. IL23 Binding Domains

The anti-TL1A×anti-IL23 antibodies provided herein include at least one IL23 binding domain (e.g., an IL23p19 binding domain). Likewise, the anti-IL23 antibodies provided herein include at least one IL23 binding domain (e.g., an IL23p19 binding domain). Any suitable IL23 binding domain can be included in the anti-TL1A×anti-IL23 antibodies or anti-IL23 antibodies provided herein. For example, the IL23 binding domain can be a human IL23 binding domain (e.g., a human IL23p19 binding domain).

Suitable IL23 binding domains can Tables a set of 6 CDRs as depicted in the tables and sequences (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Tables 11 and 25), either as they are underlined or, in the case where a different numbering scheme is used as described herein and as shown in Table 3, as the CDRs that are identified using other alignments within the variable heavy (VH) domain and variable light domain (VL) sequences of those depicted in Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Tables 11 and 25. Suitable TL23 ABDs can also include the entire VH and VL sequences as depicted in these sequences and tables, used as scFvs or as Fabs.

In one embodiment, the TL23 antigen-binding domain includes the 6 CDRs (i.e., vhCDR1-3 and vlCDR1-3) of any of the TL23 binding domains described herein, including the tables and sequences (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Tables 11 and 25). In some embodiments, the IL23 ABD is IL23-A[IL23]_H1L1 (SEQ ID NOS: 177 and 178 and Table 11). In some embodiments, the IL23 ABD is an IL23 ABD as disclosed in Table 44 or Table 24. Table 11 depicts the variable heavy and variable light domains of an illustrative IL23 antibody. IL23-A[IL23] binding domain specifically binds to the p19 subunit of IL-23.

TABLE 11
IL23-A[IL23]_H1L1

| | Sequence | SEQ ID NO: |
|---|---|---|
| Variable | QVQLVQSGAEVKKPGSSVKVSCKASGYTFTDQTIHWMRQAPGQGLEWIG | 179 |
| Heavy (vh) | YIYPRDDSPKYNENFKGKVTITADKSTSTAYMELSSLRSEDTAVYYCAIPD | |
| Domain | RSGYAWFIYWGQGTLVTVSS | |
| vhCDR1 | DQTIH | 180 |
| vhCDR2 | YIYPRDDSPKYNENFKG | 181 |
| vhCDR3 | PDRSGYAWFIY | 182 |
| Variable Light | DIQMTQSPSSLSASVGDRVTITCKASRDVAIAVAWYQQKPGKVPKLLIYW | 183 |
| (vl) Domain | ASTRHTGVPSRFSGSGSRTDFTLTISSLQPEDVADYFCHQYSSYPFTFGSGT | |
| | KLEIK | |
| VlCDR1 | KASRDVAIAVA | 184 |
| VlCDR2 | WASTRHT | 115 |
| VlCDR3 | HQYSSYPFT | 185 |

In addition to the parental CDR sets disclosed in the tables that form the IL23 ABDs (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Tables 11 and 25), provided herein are variant IL23 ABDs having CDRs that include at least one modification of the IL23 ABD CDRs disclosed herein (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Tables 11 and 25). In one embodiment, the TL23 ABD includes a set of 6 CDRs with 1, 2, 3, 4, 5, 6, 7, 8, 9, 10 amino acid modifications as compared to the 6 CDRs of a IL23 ABD as described herein, including the tables or sequences (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Tables 11 and 25). That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In exemplary embodiments, the IL23 ABD includes a set of 6 CDRs with 1, 2, 3, 4, 5, 6, 7, 8, 9, 10 amino acid modifications as compared to the 6 CDRs of IL23-A[IL23]_H1L1 (Table 11). In exemplary embodiments, the IL23 ABD includes a set of 6 CDRs with 1, 2, 3, 4, 5, 6, 7, 8, 9, 10 amino acid modifications as compared to the 6 CDRs of an IL23 ABD in Table 44 or 24. In certain embodiments, the IL23 ABD is capable of binding IL23 antigen, as measured by at least one of a Biacore, surface plasmon resonance (SPR) and/or BLI (biolayer interferometry, e.g., Octet assay) assay, with the latter finding particular use in many embodiments. In particular embodiments, the IL23 ABD is capable of binding human IL23p19 antigen (see SEQ ID NO: 1). In some embodiments, the VL sequence includes 66V (i.e., a V at position 66) and/or 105Y (i.e., a Y at position 105) as numbered by Kabat. In some embodiments, the VL sequence includes 66V and 105Y.

In some embodiments, the IL23 ABD of the subject anti-TL1A×anti-IL23 antibody includes 6 CDRs that are at least 90, 95, 97, 98 or 99% identical to the 6 CDRs of a IL23 ABD as described herein, including the tables and sequences (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Tables 11 and 25). In exemplary embodiments, the IL23 ABD includes 6 CDRs that are at least 90, 95, 97, 98 or 99% identical to the 6 CDRs of IL23-A[IL23]_H1L1 (Table 11). In exemplary embodiments, the IL23 ABD includes 6 CDRs that are at least 90, 95, 97, 98 or 99% identical to the 6 CDRs of an IL23 ABD in Table 44 or 24. In certain embodiments, the IL23 ABD is capable of binding IL23 antigen, as measured by at least one of a Biacore, surface plasmon resonance (SPR) and/or BLI (biolayer interferometry, e.g., Octet assay) assay, with the latter finding particular use in many embodiments. In particular embodiments, the IL23 ABD is capable of binding human IL23p19 antigen (see SEQ ID NO: 1). In some embodiments, the VL sequence includes 66V (i.e., a V at position 66) and/or 105Y (i.e., a Y at position 105) as numbered by Kabat. In some embodiments, the VL sequence includes 66V and 105Y.

In another exemplary embodiment, the IL23 ABD of the subject anti-TL1a×anti-IL23 antibody includes the variable heavy (VH) domain and variable light (VL) domain of any one of the IL23 ABDs described herein, including the tables and sequences (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Tables 11 and 25). In exemplary embodiments, the IL23 ABD is IL23-A[IL23]_H1L1 (Table 11). In exemplary embodiments, the IL23 ABD is an IL23 ABD in Table 44 or 24.

In some embodiments, the anti-TL1A×anti-IL23 antibody includes a IL23 ABD that includes a variable heavy domain and/or a variable light domain that are variants of a TL1AABD VH and VL domain disclosed herein (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Tables 11 and 25). In one embodiment, the variant VH domain and/or VL domain has from 1, 2, 3, 4, 5, 6, 7, 8, 9 or 10 amino acid changes from a VH and/or VL domain of a IL23 ABD described herein, including the tables and sequences (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Tables 11 and 25). In some embodiments, the variant VH domain and/or VL domain has includes 1, 2, 3, 4, 5, 6, 7, 8, 9 or 10 amino acid changes in a framework region (e.g., FR1, FR2, FR3, or FR4) from a VH and/or VL domain of a IL23 ABD described herein, including the tables and sequences (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Tables 11 and 25). In some embodiments, the variant VH and/or VL domain does not have any changes in the CDRs from a VH and/or VL domain of an IL23 ABD described herein but has changes in a framework region. In some embodiments, the variant VH and/or VL domain includes 1, 2, 3, 4, 5, 6, 7, 8, 9 or 10 amino acid changes in a framework region (e.g., FR1, FR2, FR3, or FR4) from a VH and/or VL domain of an IL23 ABD described herein, wherein the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In exemplary embodiments, the variant VH domain and/or VL domain has from 1, 2, 3, 4, 5, 6, 7, 8, 9 or 10 amino acid changes from a VH and/or VL domain of IL23-A[IL23]_H1L1 (Table 11). In exemplary embodiments, the variant VH domain and/or VL domain has from 1, 2, 3, 4, 5, 6, 7, 8, 9 or 10 amino acid changes from a VH and/or VL domain of an IL23 ABD in Table 44 or 24. In exemplary embodiments, the variant VH domain and/or VL domain has from 1, 2, 3, 4, 5, 6, 7, 8, 9 or 10 amino acid changes in a framework region (e.g., FR1, FR2, FR3, or FR4) of the VH and/or VL domain of IL23-A[IL23]_H1L1 (Table 11). In certain embodiments, the IL23 ABD is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the IL23 ABD is capable of binding human IL23p19 antigen (see SEQ ID NO: 1). In some embodiments, the VL sequence includes 66V (i.e., a V at position 66) and/or 105Y (i.e., a Y at position 105) as numbered by Kabat. In some embodiments, the VL sequence includes 66V and 105Y.

In one embodiment, the variant VH and/or VL domain is at least 90, 95, 97, 98 or 99% identical to the VH and/or VL of an IL23 ABD as described herein, including the tables and sequences (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Tables 11 and 25). In exemplary embodiments, the variant VH and/or VL domain is at least 90, 95, 97, 98 or 99% identical to the VH and/or VL of one of IL23-A[IL23]_H1L1 (Table 11). In exemplary embodiments, the variant VH and/or VL domain is at least 90, 95, 97, 98 or 99% identical to a VH and/or VL of an IL23 ABD Table 44 or 24. In some embodiments, the variant VH and/or VL domain does not have any changes in the CDRs from a VH and/or VL domain of an IL23 ABD described herein but has changes in a framework region. In some embodiments, the variant VH and/or VL domain includes amino acid changes in a framework region (e.g., FR1, FR2, FR3, or FR4) from a VH and/or VL domain of an IL23 ABD described herein, wherein the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In certain embodiments, the IL23 ABD is capable of binding to IL23 (e.g., IL23p19), as measured by at least one of a Biacore, surface plasmon resonance (SPR) and/or BLI (biolayer interferometry, e.g., Octet assay) assay, with the latter finding particular use in many embodiments. In particular embodiments, the IL23 ABD is capable of binding human IL23p19 antigen (see SEQ ID NO: 1). In some embodiments, the VL sequence includes 66V (i.e., a V at position 66) and/or 105Y (i.e., a Y at position 105) as numbered by Kabat. In some embodiments, the VL sequence includes 66V and 105Y.

In some embodiments, an IL23 binding domain comprises a variable heavy domain comprising a vhCDR1, a vhCDR2, and a vhCDR3 contained within a variable heavy domain comprising the amino acid sequence of any one of SEQ ID NOS: 2233, 2235, and 179, and a variable light domain comprising a vlCDR1, a vlCDR2, and a vlCDR3 contained within a variable light domain comprising the amino acid sequence of any one of SEQ ID NOS: 2234, 2237, and 1248. In some embodiments, an IL23 binding domain comprises a variable heavy domain comprising a vhCDR1, a vhCDR2, and a vhCDR3 contained within a variable heavy domain comprising the amino acid sequence of SEQ ID NO: 2233, and a variable light domain comprising a vlCDR1, a vlCDR2, and a vlCDR3 contained within a variable light domain comprising the amino acid sequence of SEQ ID NO: 2234. In some embodiments, an IL23 binding domain comprises a variable heavy domain comprising a vhCDR1, a vhCDR2, and a vhCDR3 contained within a variable heavy domain comprising the amino acid sequence of SEQ ID NO: 2235, and a variable light domain comprising a vlCDR1, a vlCDR2, and a vlCDR3 contained within a variable light domain comprising the amino acid sequence of SEQ ID NO: 2237. In some embodiments, an TL23 binding domain comprises a variable heavy domain comprising a vhCDR1, a vhCDR2, and a vhCDR3 contained within a variable heavy domain comprising the amino acid sequence of SEQ ID NO: 179, and a variable light domain comprising a vlCDR1, a vlCDR2, and a vlCDR3 contained within a variable light domain comprising the amino acid sequence of SEQ ID NO: 1248. In some embodiments, an IL23 binding domain comprises a variable heavy domain comprising a vhCDR1, a vhCDR2, and a vhCDR3 contained within a variable heavy domain comprising the amino acid sequence of SEQ ID NO: 2113, and a variable light domain comprising a vlCDR1, a vlCDR2, and a vlCDR3 contained within a variable light domain comprising the amino acid sequence of SEQ ID NO: 2231. In some embodiments, an IL23 binding domain comprises a variable heavy domain comprising a vhCDR1, a vhCDR2, and a vhCDR3 contained within a variable heavy domain comprising the amino acid sequence of SEQ ID NO: 1967, and a variable light domain comprising a vlCDR1, a vlCDR2, and a vlCDR3 contained within a variable light domain comprising the amino acid sequence of SEQ ID NO: 1248. In some embodiments, an IL23 binding domain comprises a variable heavy domain comprising a vhCDR1, a vhCDR2, and a vhCDR3 contained within any pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some embodiments, the CDRs are as defined by Kabat. In other embodiments, the CDRs are as defined by Chothia, Kabat+Chothia, AbM, contact, or IMGT. In some embodiments, an IL23 binding domain comprises a vhCDR1 comprising the amino acid sequence of SEQ ID NO: 180, a vhCDR2 comprising the amino acid sequence of SEQ ID NO: 181, a vhCDR3 comprising the amino acid sequence of SEQ ID NO: 182 or 2236, a vlCDR1 comprising the amino acid sequence of SEQ ID NO: 184, a vlCDR2 comprising the amino acid sequence of SEQ ID NO: 1249 or 2238, and a vlCDR3 comprising the amino acid sequence of SEQ ID NO: 1250. In some embodiments, an TL23 binding domain comprises a vhCDR1 comprising the amino acid sequence of SEQ ID NO: 180, a vhCDR2 comprising the amino acid sequence of SEQ ID NO: 181, a vhCDR3 comprising the amino acid sequence of SEQ ID NO: 182, a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some embodiments, an IL23 binding domain comprises a vhCDR1 comprising the amino acid sequence of SEQ ID NO: 180, a vhCDR2 comprising the amino acid sequence of SEQ ID NO: 181, a vhCDR3 comprising the amino acid sequence of SEQ ID NO: 2236, vlCDR1 comprising the amino acid sequence of SEQ ID NO: 184, a vlCDR2 comprising the amino acid sequence of SEQ ID NO: 2238, and a vlCDR3 comprising the amino acid sequence of SEQ ID NO: 1250. In some embodiments, an IL23 binding domain comprises a vhCDR1 comprising the amino acid sequence of SEQ ID NO: 2239, a vhCDR2 comprising the amino acid sequence of SEQ ID NO: 181, a vhCDR3 comprising the amino acid sequence of SEQ ID NO: 182, a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some embodiments, an TL23 binding domain comprises a set of 6 CDRs with 1, 2, 3, 4, 5, 6, 7, 8, 9, 10 amino acid modifications as compared to the 6 CDRs of any of the above IL23 binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, an IL23 binding domain comprises a set of 6 CDRs that are at least 90, 95, 97, 98 or 99% identical to the 6 CDRs of any of the above IL23 binding domains. In some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 antigen binding domain is capable of binding IL23 antigen, as measured by at least one of a Biacore, surface plasmon resonance (SPR) and/or BLI (biolayer interferometry, e.g., Octet assay) assay, with the latter finding particular use in many embodiments. In particular embodiments, the IL23 binding domain is capable of binding human IL23p19 antigen (see SEQ ID NO: 1). In particular embodiments, the antibody specifically binds human IL23 with a K_{D} of less than about 1.1E-10 M as measured by surface plasmon resonance at 25° C., optionally wherein the antibody specifically binds human IL23 with a K_{D} of less than about 6E-11 M, less than about 5.9E-11 M, less than about 5.8E-11 M, or less than about 5.7E-11 M. In some embodiments, an antibody disclosed herein specifically binds human TL23 with a K_{D} of between about 2.4E-11 and about 1E-10 M or between about 2.4E-11 and about 5.7E-11 M as measured by surface plasmon resonance or Biacore at 25° C. In some embodiments, an antibody disclosed herein specifically binds human IL23 with a K_{D} of between about 5E-11 and about 6E-11 M as measured by surface plasmon resonance or Biacore at 25° C. In particular embodiments, the antibody inhibits TL23 activity in an TL23 luciferase reporter assay with an IC₅₀ of less than about 65 pM, less than about 64 pM, or less than about 63 pM, optionally wherein the antibody inhibits TL23 activity with an IC₅₀ of less than about 60 pM, less than about 59 pM, less than about 58 pM, less than about 57 pM, or less than about 56 pM. In some embodiments, an antibody disclosed herein inhibits IL23 activity in an IL23 luciferase reporter assay with an IC₅₀ of between about 47 pM and about 63 pM or between about 50 pM and about 60 pM.

In some embodiments, the variable heavy domain comprises the amino acid sequence of any one of SEQ ID NOS: 2233, 2235, and 179, and the variable light domain comprises the amino acid sequence of any one of SEQ ID NOS: 2234, 2237, and 1248. In some embodiments, the variable heavy domain comprises the amino acid sequence of SEQ ID NO: 2233, and the variable light domain comprises the amino acid sequence of SEQ ID NO: 2234. In some embodiments, the variable heavy domain comprises the amino acid sequence of SEQ ID NO: 2235, and the variable light domain comprises the amino acid sequence of SEQ ID NO: 2237. In some embodiments, the variable heavy domain comprises the amino acid sequence of SEQ ID NO: 179, and the variable light domain comprises the amino acid sequence of SEQ ID NO: 1248. In some embodiments, the variable heavy domain comprises the amino acid sequence of SEQ ID NO: 2113, and the variable light domain comprises the amino acid sequence of SEQ ID NO: 2231. In some embodiments, the variable heavy domain comprises the amino acid sequence of SEQ ID NO: 1967, and the variable light domain comprises the amino acid sequence of SEQ ID NO: 1248. In some embodiments, the variable heavy domain comprises the amino acid sequence of any one of SEQ ID NOS: 1927-2132, and the variable light domain comprises the amino acid sequence of any one of SEQ ID NOS: 2133-2232. In some embodiments, the variable heavy domain and the variable light domain comprise the pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some embodiments, the variable heavy domain and the variable light domain comprise any pair of anti-IL23 variable heavy domain and variable light domain in Table 29 or 33. In some such antibodies, the variable heavy domain or the variable light domain can comprise variants of any of the above in which an N-terminal glutamine or glutamate is replaced with pyroglutamate. In some embodiments, the IL23 binding domain comprises a variant VH domain and/or variant VL domain having 1, 2, 3, 4, 5, 6, 7, 8, 9 or 10 amino acid changes from any of the above VH and VL domains. In some embodiments, the IL23 binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 binding domain is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the IL23 binding domain is capable of binding human IL23p19 antigen (see SEQ ID NO: 1). In particular embodiments, the antibody specifically binds human IL23 with a K_{D} of less than about 1.1E-10 M as measured by surface plasmon resonance at 25° C., optionally wherein the antibody specifically binds human TL23 with a K_{D} of less than about 6E-11 M, less than about 5.9E-11 M, less than about 5.8E-11 M, or less than about 5.7E-11 M. In some embodiments, an antibody disclosed herein specifically binds human IL23 with a K_{D} of between about 2.4E-11 and about 1E-10 M or between about 2.4E-11 and about 5.7E-11 M as measured by surface plasmon resonance or Biacore at 25° C. In some embodiments, an antibody disclosed herein specifically binds human IL23 with a K_{D} of between about 5E-11 and about 6E-11 M as measured by surface plasmon resonance or Biacore at 25° C. In particular embodiments, the antibody inhibits IL23 activity in an IL23 luciferase reporter assay with an IC₅₀ of less than about 65 pM, less than about 64 pM, or less than about 63 pM, optionally wherein the antibody inhibits IL23 activity with an IC₅₀ of less than about 60 pM, less than about 59 pM, less than about 58 pM, less than about 57 pM, or less than about 56 pM. In some embodiments, an antibody disclosed herein inhibits IL23 activity in an IL23 luciferase reporter assay with an IC₅₀ of between about 47 pM and about 63 pM or between about 50 pM and about 60 pM.

In some embodiments, an TL23 binding domain comprises a variable heavy domain comprising a vhCDR1, a vhCDR2, and a vhCDR3 contained within a variable heavy domain comprising the amino acid sequence of SEQ ID NO: 2233, and a variable light domain comprising a vlCDR1, a vlCDR2, and a vlCDR3 contained within a variable light domain comprising the amino acid sequence of SEQ ID NO: 2234. In some embodiments, the CDRs are as defined by Kabat. In other embodiments, the CDRs are as defined by Chothia, Kabat+Chothia, AbM, contact, or IMGT. In some embodiments, the variable heavy domain comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and the variable light domain comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some embodiments, an IL23 binding domain comprises a set of 6 CDRs with 1, 2, 3, 4, 5, 6, 7, 8, 9, 10 amino acid modifications as compared to the 6 CDRs of any of the above IL23 binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, an IL23 binding domain comprises a set of 6 CDRs that are at least 90, 95, 97, 98 or 99% identical to the 6 CDRs of any of the above TL23 binding domains. In some embodiments, the variable heavy domain comprises the amino acid sequence of SEQ ID NO: 2233, and the variable light domain comprises the amino acid sequence of SEQ ID NO: 2234. In some such antibodies, the variable heavy domain or the variable light domain can comprise variants of any of the above in which an N-terminal glutamine or glutamate is replaced with pyroglutamate. In some embodiments, the IL23 binding domain comprises a variant VH domain and/or variant VL domain having 1, 2, 3, 4, 5, 6, 7, 8, 9 or 10 amino acid changes from any of the above VH and VL domains. In some embodiments, the IL23 binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 binding domain is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the IL23 binding domain is capable of binding human IL23p19 antigen (see SEQ ID NO: 1).

In some embodiments, an IL23 binding domain comprises a variable heavy domain comprising a vhCDR1, a vhCDR2, and a vhCDR3 contained within a variable heavy domain comprising the amino acid sequence of SEQ ID NO: 2235, and a variable light domain comprising a vlCDR1, a vlCDR2, and a vlCDR3 contained within a variable light domain comprising the amino acid sequence of SEQ ID NO: 2237. In some embodiments, the CDRs are as defined by Kabat. In other embodiments, the CDRs are as defined by Chothia, Kabat+Chothia, AbM, contact, or IMGT. In some embodiments, the variable heavy domain comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 2236, and the variable light domain comprises a vlCDR1 comprising the amino acid sequence of SEQ ID NO: 184, a vlCDR2 comprising the amino acid sequence of SEQ ID NO: 2238, and a vlCDR3 comprising the amino acid sequence of SEQ ID NO: 1250. In some embodiments, an IL23 binding domain comprises a set of 6 CDRs with 1, 2, 3, 4, 5, 6, 7, 8, 9, 10 amino acid modifications as compared to the 6 CDRs of any of the above IL23 binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, an TL23 binding domain comprises a set of 6 CDRs that are at least 90, 95, 97, 98 or 99% identical to the 6 CDRs of any of the above IL23 binding domains. In some embodiments, the variable heavy domain comprises the amino acid sequence of SEQ ID NO: 2235, and the variable light domain comprises the amino acid sequence of SEQ ID NO: 2237. In some such antibodies, the variable heavy domain or the variable light domain can comprise variants of any of the above in which an N-terminal glutamine or glutamate is replaced with pyroglutamate. In some embodiments, the TL23 binding domain comprises a variant VH domain and/or variant VL domain having 1, 2, 3, 4, 5, 6, 7, 8, 9 or 10 amino acid changes from any of the above VH and VL domains. In some embodiments, the TL23 binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 binding domain is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the IL23 binding domain is capable of binding human IL23p19 antigen (see SEQ ID NO: 1).

In some embodiments, an IL23 binding domain comprises a variable heavy domain comprising a vhCDR1, a vhCDR2, and a vhCDR3 contained within a variable heavy domain comprising the amino acid sequence of SEQ ID NO: 179, and a variable light domain comprising a vlCDR1, a vlCDR2, and a vlCDR3 contained within a variable light domain comprising the amino acid sequence of SEQ ID NO: 1248. In some embodiments, the CDRs are as defined by Kabat. In other embodiments, the CDRs are as defined by Chothia, Kabat+Chothia, AbM, contact, or IMGT. In some embodiments, the variable heavy domain comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and the variable light domain comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some embodiments, an IL23 binding domain comprises a set of 6 CDRs with 1, 2, 3, 4, 5, 6, 7, 8, 9, 10 amino acid modifications as compared to the 6 CDRs of any of the above IL23 binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, an IL23 binding domain comprises a set of 6 CDRs that are at least 90, 95, 97, 98 or 99% identical to the 6 CDRs of any of the above TL23 binding domains. In some embodiments, the variable heavy domain comprises the amino acid sequence of SEQ ID NO: 179, and the variable light domain comprises the amino acid sequence of SEQ ID NO: 1248. In some such antibodies, the variable heavy domain or the variable light domain can comprise variants of any of the above in which an N-terminal glutamine or glutamate is replaced with pyroglutamate. In some embodiments, the IL23 binding domain comprises a variant VH domain and/or variant VL domain having 1, 2, 3, 4, 5, 6, 7, 8, 9 or 10 amino acid changes from any of the above VH and VL domains. In some embodiments, the TL23 binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 binding domain is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the TL23 binding domain is capable of binding human IL23p19 antigen (see SEQ ID NO: 1).

In some embodiments, an anti-IL23 antibody or an anti-TL1A×anti-IL23 antibody comprises a heavy chain comprising the amino acid sequence of SEQ ID NO: 2244 or a variant thereof in which the N-terminal glutamine is replaced with pyroglutamate and/or the C-terminal lysine or the C-terminal lysine and glycine are removed, and a light chain comprising the amino acid sequence of SEQ ID NO: 2245. In some embodiments, an anti-IL23 antibody or an anti-TL1A×anti-IL23 antibody comprises a heavy chain comprising the amino acid sequence of SEQ ID NO: 2250 or a variant thereof in which the N-terminal glutamine is replaced with pyroglutamate and/or the C-terminal lysine or the C-terminal lysine and glycine are removed, and a light chain comprising the amino acid sequence of SEQ ID NO: 2251. In some embodiments, an anti-IL23 antibody or an anti-TL1A×anti-IL23 antibody comprises a heavy chain comprising the amino acid sequence of SEQ ID NO: 2266 or a variant thereof in which the N-terminal glutamine is replaced with pyroglutamate and/or the C-terminal lysine or the C-terminal lysine and glycine are removed, and a light chain comprising the amino acid sequence of SEQ ID NO: 2267. In some embodiments, an anti-IL23 antibody or an anti-TL1A×anti-IL23 antibody comprises an anti-IL23 heavy chain and light chain pair from XENP53412, XENP53413, XENP53369, XENP53375, XENP53387, XENP53415, XENP53370, XENP53371, XENP53376, XENP53377, XENP53378, XENP53379, XENP53380, or XENP53414, or heavy chain variants thereof in which the N-terminal glutamine is replaced with pyroglutamate and/or the C-terminal lysine or the C-terminal lysine and glycine are removed. In some embodiments, an anti-IL23 antibody or an anti-TL1A×anti-IL23 antibody comprises a heavy chain and/or a light chain having 1, 2, 3, 4, 5, 6, 7, 8, 9 or 10 amino acid changes from any of the above heavy chains and light chains. In some embodiments, an anti-IL23 antibody or an anti-TL1A×anti-IL23 antibody comprises a heavy chain and/or a light chain at least 90%, 95%, 97%, 98%, or 99% identical to any of the above heavy chains and light chains. In some embodiments, the VL sequence includes 66V (i.e., a V at position 66) and/or 105Y (i.e., a Y at position 105) as numbered by Kabat. In some embodiments, the VL sequence includes 66V and 105Y. In some embodiments, the amino acid changes are in a constant region but not in a variable region. In some embodiments, the amino acid changes are in a constant region or in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. In some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 binding domain is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the IL23 binding domain is capable of binding human IL23p19 antigen (see SEQ ID NO: 1).

### C. Chimeric and Humanized Antibodies

In certain embodiments, the subject antibodies provided herein include a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized antibody may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention).

In one embodiment, the parent antibody has been affinity matured. Structure-based methods may be employed for humanization and affinity maturation, for example as described in US 2006-0008883 A1, incorporated by reference in its entirety for all purposes. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### D. Bispecific and Heterodimeric Antibodies

Provided herein are bispecific and heterodimeric anti-TL1A×anti-IL23 antibodies comprising a TL1A binding domain and an IL23 binding domain as described elsewhere herein.

In exemplary embodiments, the anti-TL1A×anti-IL23 antibodies provided herein are heterodimeric bispecific antibodies that include two variant Fc domain sequences. Such variant Fc domains include amino acid modifications to facilitate the self-assembly and/or purification of the heterodimeric antibodies. Such bispecific antibodies can also include variant heavy chain constant domain sequences (e.g., CH1) and variant light chain constant domain sequences. Likewise, such bispecific antibodies can also include variant variable heavy domain sequences and variant variable light domain sequences. Such variant sequences can include, for example, amino acid modifications to facilitate the self-assembly of the heterodimeric antibodies (e.g., favor heterodimeric formation and/or correct heavy chain/light chain pairing).

Exemplary bispecific antibodies are provided in Tables 44 and 29-34. For example, an anti-TL1A×anti-IL23 antibody can comprise the TL1A binding domain and the IL23 binding domain from any bispecific antibody in Tables 44 and 29-34 or variants thereof as described elsewhere herein.

In some embodiments, the anti-TL1A variable heavy domain and the variable light domain and the anti-IL23 variable heavy domain and variable light domain comprise the set of CDRs in the pair of anti-TL1A variable heavy domain and variable light domain and the pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some embodiments, the anti-TL1A variable heavy domain and the variable light domain and the anti-IL23 variable heavy domain and variable light domain comprise the set of CDRs in the pair of anti-TL1A variable heavy domain and variable light domain and the pair of anti-IL23 variable heavy domain and variable light domain in an anti-TL1A×anti-IL23 antibody in Table 44. In some embodiments, the CDRs are as defined by Kabat. In other embodiments, the CDRs are as defined by Chothia, Kabat+Chothia, AbM, contact, or IMGT. In some embodiments, a TL1A binding domain or an anti-IL23 binding domain comprises a set of 6 CDRs with 1, 2, 3, 4, 5, 6, 7, 8, 9, 10 amino acid modifications as compared to the 6 CDRs of any of the above TL1A binding or IL23 binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, a TL1A binding domain or IL23 binding domain comprises a set of 6 CDRs that are at least 90, 95, 97, 98 or 99% identical to the 6 CDRs of any of the above TL1A binding domains or IL23 binding domains. Regarding the TL1A binding domain, in some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4). In particular embodiments, the antibody specifically binds human TL1A with a K_{D} of less than about 6.5E-11 M, less than about 6.4 E-11 M, or less than about 6.3E-11 as measured by surface plasmon resonance at 25° C., optionally wherein the antibody specifically binds human TL1A with a K_{D} of less than about 3E-11 M, less than about 2.9E-11 M, less than about 2.8E-11 M, or less than about 2.7E-11 M. In some embodiments, an antibody disclosed herein specifically binds human TL1A with a K_{D} of between about 2E-11 M and about 7E-11M, between about 2.5E-11 M and about 7E-11 M, or between about 2.5E-11 M and about 6.5E-11 M as measured by surface plasmon resonance or Biacore at 25° C. In some embodiments, an antibody disclosed herein specifically binds human TL1A with a K_{D} of between about 2.5E-11 M and about 3.5E-11 M, between about 2.5E-11 M and about 3E-11 M, or between about 2.5E-11 M and about 2.7E-11 M as measured by surface plasmon resonance or Biacore at 25° C. In particular embodiments, the antibody the antibody inhibits TL1A activity in a TL1A luciferase reporter assay with an IC₅₀ of less than about 4.5 nM. In some embodiments, an antibody disclosed herein inhibits TL1A activity in a TL1A luciferase reporter assay with an IC₅₀ of between about 2.5 nM and about 4.5 nM, between about 3.5 nM and about 5.5 nM, or between about 4 nM and about 5 nM. Regarding the TL23 binding domain, in some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 antigen binding domain is capable of binding IL23 antigen, as measured by at least one of a Biacore, surface plasmon resonance (SPR) and/or BLI (biolayer interferometry, e.g., Octet assay) assay, with the latter finding particular use in many embodiments. In particular embodiments, the IL23 binding domain is capable of binding human IL23p19 antigen (see SEQ ID NO: 1). In particular embodiments, the antibody specifically binds human IL23 with a K_{D} of less than about 1.1E-10 M as measured by surface plasmon resonance at 25° C., optionally wherein the antibody specifically binds human IL23 with a K_{D} of less than about 6E-11 M, less than about 5.9E-11 M, less than about 5.8E-11 M, or less than about 5.7E-11 M. In some embodiments, an antibody disclosed herein specifically binds human IL23 with a K_{D} of between about 2.4E-11 and about 1E-10 M or between about 2.4E-11 and about 5.7E-11 M as measured by surface plasmon resonance or Biacore at 25° C. In some embodiments, an antibody disclosed herein specifically binds human IL23 with a K_{D} of between about 5E-11 and about 6E-11 M as measured by surface plasmon resonance or Biacore at 25° C. In particular embodiments, the antibody inhibits IL23 activity in an IL23 luciferase reporter assay with an IC₅₀ of less than about 65 pM, less than about 64 pM, or less than about 63 pM, optionally wherein the antibody inhibits TL23 activity with an IC₅₀ of less than about 60 pM, less than about 59 pM, less than about 58 pM, less than about 57 pM, or less than about 56 pM. In some embodiments, an antibody disclosed herein inhibits IL23 activity in an IL23 luciferase reporter assay with an IC₅₀ of between about 47 pM and about 63 pM or between about 50 pM and about 60 pM.

In some embodiments, the anti-TL1A variable heavy domain and the variable light domain and the anti-IL23 variable heavy domain and variable light domain comprise the pair of anti-TL1A variable heavy domain and variable light domain and the pair of anti-IL23 variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414. In some embodiments, the anti-TL1A variable heavy domain and the variable light domain and the anti-IL23 variable heavy domain and variable light domain comprise the pair of anti-TL1A variable heavy domain and variable light domain and the pair of anti-IL23 variable heavy domain and variable light domain in Table 44. In some such antibodies, the anti-TL1A variable heavy domain or variable light domain or the anti-IL23 variable heavy domain or variable light domain can comprise variants of any of the above in which an N-terminal glutamine or glutamate is replaced with pyroglutamate. In some embodiments, the TL1A binding domain or the TL23 binding domain comprises a variant VH domain and/or variant VL domain having 1, 2, 3, 4, 5, 6, 7, 8, 9 or 10 amino acid changes from any of the above VH and VL domains. In some embodiments, the TL1A binding domain or the IL23 binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. Regarding the TL1A binding domain, in some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4). In particular embodiments, the antibody specifically binds human TL1A with a K_{D} of less than about 6.5E-11 M, less than about 6.4 E-11 M, or less than about 6.3E-11 as measured by surface plasmon resonance at 25° C., optionally wherein the antibody specifically binds human TL1A with a K_{D} of less than about 3E-11 M, less than about 2.9E-11 M, less than about 2.8E-11 M, or less than about 2.7E-11 M. In some embodiments, an antibody disclosed herein specifically binds human TL1A with a K_{D} of between about 2E-11 M and about 7E-11M, between about 2.5E-11 M and about 7E-11 M, or between about 2.5E-11 M and about 6.5E-11 M as measured by surface plasmon resonance or Biacore at 25° C. In some embodiments, an antibody disclosed herein specifically binds human TL1A with a K_{D} of between about 2.5E-11 M and about 3.5E-11 M, between about 2.5E-11 M and about 3E-11 M, or between about 2.5E-11 M and about 2.7E-11 M as measured by surface plasmon resonance or Biacore at 25° C. In particular embodiments, the antibody the antibody inhibits TL1A activity in a TL1A luciferase reporter assay with an IC₅₀ of less than about 4.5 nM. In some embodiments, an antibody disclosed herein inhibits TL1A activity in a TL1A luciferase reporter assay with an IC₅₀ of between about 2.5 nM and about 4.5 nM, between about 3.5 nM and about 5.5 nM, or between about 4 nM and about 5 nM. Regarding the IL23 binding domain, in some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 binding domain is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the IL23 binding domain is capable of binding human IL23p19 antigen (see SEQ ID NO: 1). In particular embodiments, the antibody specifically binds human IL23 with a K_{D} of less than about 1.1E-10 M as measured by surface plasmon resonance at 25° C., optionally wherein the antibody specifically binds human IL23 with a K_{D} of less than about 6E-11 M, less than about 5.9E-11 M, less than about 5.8E-11 M, or less than about 5.7E-11 M. In some embodiments, an antibody disclosed herein specifically binds human IL23 with a K_{D} of between about 2.4E-11 and about 1E-10 M or between about 2.4E-11 and about 5.7E-11 M as measured by surface plasmon resonance or Biacore at 25° C. In some embodiments, an antibody disclosed herein specifically binds human IL23 with a K_{D} of between about 5E-11 and about 6E-11 M as measured by surface plasmon resonance or Biacore at 25° C. In particular embodiments, the antibody inhibits IL23 activity in an IL23 luciferase reporter assay with an IC₅₀ of less than about 65 pM, less than about 64 pM, or less than about 63 pM, optionally wherein the antibody inhibits IL23 activity with an IC₅₀ of less than about 60 pM, less than about 59 pM, less than about 58 pM, less than about 57 pM, or less than about 56 pM. In some embodiments, an antibody disclosed herein inhibits IL23 activity in an IL23 luciferase reporter assay with an IC₅₀ of between about 47 pM and about 63 pM or between about 50 pM and about 60 pM.

In some embodiments, the anti-TL1A variable heavy domain and variable light domain are referred to as VH1 and VL1, and the anti-IL23 variable heavy domain and variable light domain are referred to as VH2 and VL2.

In some embodiments, the VH1 comprises a vhCDR1, a vhCDR2, and a vhCDR3 contained within a variable heavy domain comprising the amino acid sequence of any one of SEQ ID NOS: 2241, 2253, 2259, 1674, and 1200, (ii) the VL1 comprises a vlCDR1, a vlCDR2, and a vlCDR3 contained within a variable light domain comprising the amino acid sequence of any one of SEQ ID NOS: 2243 and 1204, (iii) the VH2 comprises a vhCDR1, a vhCDR2, and a vhCDR3 contained within a variable heavy domain comprising the amino acid sequence of any one of SEQ ID NOS: 2233, 2235, and 179, and (iv) the VL2 comprises a vlCDR1, a vlCDR2, and a vlCDR3 contained within a variable light domain comprising the amino acid sequence of any one of SEQ ID NOS: 2234, 2237, and 1248. In some embodiments, the CDRs are as defined by Kabat. In other embodiments, the CDRs are as defined by Chothia, Kabat+Chothia, AbM, contact, or IMGT. In some embodiments, the VH1 comprises a vhCDR1 comprising the amino acid sequence of SEQ ID NO: 102 or 1201, a vhCDR2 comprising the amino acid sequence of SEQ ID NO: 1924 or 1202, and a vhCDR3 comprising the amino acid sequence of SEQ ID NO: 1203 or 104, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182 or 2236, and (iv) the VL2 comprises a vlCDR1 comprising the amino acid sequence of SEQ ID NO: 184, a vlCDR2 comprising the amino acid sequence of SEQ ID NO: 1249 or 2238, and a vlCDR3 comprising the amino acid sequence of SEQ ID NO: 1250. In some embodiments, a binding domain comprises a set of 6 CDRs with 1, 2, 3, 4, 5, 6, 7, 8, 9, 10 amino acid modifications as compared to the 6 CDRs of any of the above binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, a binding domain comprises a set of 6 CDRs that are at least 90, 95, 97, 98 or 99% identical to the 6 CDRs of any of the above binding domains. In some embodiments, the VH1 comprises the amino acid sequence of any one of SEQ ID NOS: 2241, 2253, 2259, 1674, and 1200, (ii) the VL1 comprises the amino acid sequence of any one of SEQ ID NOS: 2243 and 1204, (iii) the VH2 comprises the amino acid sequence of any one of SEQ ID NOS: 2233, 2235, and 179, and (iv) the VL2 comprises the amino acid sequence of any one of SEQ ID NOS: 2234, 2237, and 1248. In some such antibodies, the variable heavy domain or the variable light domain can comprise variants of any of the above in which an N-terminal glutamine or glutamate is replaced with pyroglutamate. In some embodiments, a binding domain comprises a variant VH domain and/or variant VL domain having 1, 2, 3, 4, 5, 6, 7, 8, 9 or 10 amino acid changes from any of the above VH and VL domains. In some embodiments, a binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. Regarding the TL1A binding domain, in some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4). Regarding the IL23 binding domain, in some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 binding domain is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the IL23 binding domain is capable of binding human IL23p19 antigen (see SEQ ID NO: 1).

In some embodiments, the VH1 comprises a vhCDR1, a vhCDR2, and a vhCDR3 contained within a variable heavy domain comprising the amino acid sequence of SEQ ID NO: 2241, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234. In some embodiments, the CDRs are as defined by Kabat. In other embodiments, the CDRs are as defined by Chothia, Kabat+Chothia, AbM, contact, or IMGT. In some embodiments, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some embodiments, a binding domain comprises a set of 6 CDRs with 1, 2, 3, 4, 5, 6, 7, 8, 9, 10 amino acid modifications as compared to the 6 CDRs of any of the above binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, a binding domain comprises a set of 6 CDRs that are at least 90, 95, 97, 98 or 99% identical to the 6 CDRs of any of the above binding domains. In some embodiments, the VH1 comprises the amino acid sequence of SEQ ID NO: 2241, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234. In some such antibodies, the variable heavy domain or the variable light domain can comprise variants of any of the above in which an N-terminal glutamine or glutamate is replaced with pyroglutamate. In some embodiments, a binding domain comprises a variant VH domain and/or variant VL domain having 1, 2, 3, 4, 5, 6, 7, 8, 9 or 10 amino acid changes from any of the above VH and VL domains. In some embodiments, a binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. Regarding the TL1A binding domain, in some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4). Regarding the IL23 binding domain, in some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 binding domain is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the IL23 binding domain is capable of binding human IL23p19 antigen (see SEQ ID NO: 1).

In some embodiments, the VH1 comprises a vhCDR1, a vhCDR2, and a vhCDR3 contained within a variable heavy domain comprising the amino acid sequence of SEQ ID NO: 2241, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2235, and (iv) the VL2 comprises a vlCDR1, a vlCDR2, and a vlCDR3 contained within a variable light domain comprising the amino acid sequence of SEQ ID NO: 2237. In some embodiments, the CDRs are as defined by Kabat. In other embodiments, the CDRs are as defined by Chothia, Kabat+Chothia, AbM, contact, or IMGT. In some embodiments, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 2236, and (iv) the VL2 comprises a vlCDR1 comprising the amino acid sequence of SEQ ID NO: 184, a vlCDR2 comprising the amino acid sequence of SEQ ID NO: 2238, and a vlCDR3 comprising the amino acid sequence of SEQ ID NO: 1250. In some embodiments, a binding domain comprises a set of 6 CDRs with 1, 2, 3, 4, 5, 6, 7, 8, 9, 10 amino acid modifications as compared to the 6 CDRs of any of the above binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, a binding domain comprises a set of 6 CDRs that are at least 90, 95, 97, 98 or 99% identical to the 6 CDRs of any of the above binding domains. In some embodiments, the VH1 comprises the amino acid sequence of SEQ ID NO: 2241, (ii) the VL1 comprises the amino acid sequence of SEQ ID NO: 2243, (iii) the VH2 comprises the amino acid sequence of SEQ ID NO: 2235, and (iv) the VL2 comprises the amino acid sequence of SEQ ID NO: 2237. In some such antibodies, the variable heavy domain or the variable light domain can comprise variants of any of the above in which an N-terminal glutamine or glutamate is replaced with pyroglutamate. In some embodiments, a binding domain comprises a variant VH domain and/or variant VL domain having 1, 2, 3, 4, 5, 6, 7, 8, 9 or 10 amino acid changes from any of the above VH and VL domains. In some embodiments, a binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. Regarding the TL1A binding domain, in some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4). Regarding the TL23 binding domain, in some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 binding domain is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the IL23 binding domain is capable of binding human IL23p19 antigen (see SEQ ID NO: 1).

In some embodiments, the VH1 comprises a vhCDR1, a vhCDR2, and a vhCDR3 contained within a variable heavy domain comprising the amino acid sequence of SEQ ID NO: 2253, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234. In some embodiments, the CDRs are as defined by Kabat. In other embodiments, the CDRs are as defined by Chothia, Kabat+Chothia, AbM, contact, or IMGT. In some embodiments, the VH1 comprises a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some embodiments, a binding domain comprises a set of 6 CDRs with 1, 2, 3, 4, 5, 6, 7, 8, 9, 10 amino acid modifications as compared to the 6 CDRs of any of the above binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, a binding domain comprises a set of 6 CDRs that are at least 90, 95, 97, 98 or 99% identical to the 6 CDRs of any of the above binding domains. In some embodiments, the VH1 comprises the amino acid sequence of SEQ ID NO: 2253, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234. In some such antibodies, the variable heavy domain or the variable light domain can comprise variants of any of the above in which an N-terminal glutamine or glutamate is replaced with pyroglutamate. In some embodiments, a binding domain comprises a variant VH domain and/or variant VL domain having 1, 2, 3, 4, 5, 6, 7, 8, 9 or 10 amino acid changes from any of the above VH and VL domains. In some embodiments, a binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. Regarding the TL1A binding domain, in some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4). Regarding the TL23 binding domain, in some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 binding domain is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the IL23 binding domain is capable of binding human IL23p19 antigen (see SEQ ID NO: 1).

In some embodiments, the VH1 comprises a vhCDR1, a vhCDR2, and a vhCDR3 contained within a variable heavy domain comprising the amino acid sequence of SEQ ID NO: 2259, (ii) the VL1 comprises a vlCDR1, a vlCDR2, and a vlCDR3 contained within a variable light domain comprising the amino acid sequence of SEQ ID NO: 2243, (iii) the VH2 comprises a vhCDR1, a vhCDR2, and a vhCDR3 contained within a variable heavy domain comprising the amino acid sequence of SEQ ID NO: 2233, and (iv) the VL2 comprises a vlCDR1, a vlCDR2, and a vlCDR3 contained within a variable light domain comprising the amino acid sequence of SEQ ID NO: 2234. In some embodiments, the CDRs are as defined by Kabat. In other embodiments, the CDRs are as defined by Chothia, Kabat+Chothia, AbM, contact, or IMGT. In some embodiments, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 104, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some embodiments, a binding domain comprises a set of 6 CDRs with 1, 2, 3, 4, 5, 6, 7, 8, 9, 10 amino acid modifications as compared to the 6 CDRs of any of the above binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, a binding domain comprises a set of 6 CDRs that are at least 90, 95, 97, 98 or 99% identical to the 6 CDRs of any of the above binding domains. In some embodiments, the VH1 comprises the amino acid sequence of SEQ ID NO: 2259, (ii) the VL1 comprises the amino acid sequence of SEQ ID NO: 2243, (iii) the VH2 comprises the amino acid sequence of SEQ ID NO: 2233, and (iv) the VL2 comprises the amino acid sequence of SEQ ID NO: 2234. In some such antibodies, the variable heavy domain or the variable light domain can comprise variants of any of the above in which an N-terminal glutamine or glutamate is replaced with pyroglutamate. In some embodiments, a binding domain comprises a variant VH domain and/or variant VL domain having 1, 2, 3, 4, 5, 6, 7, 8, 9 or 10 amino acid changes from any of the above VH and VL domains. In some embodiments, a binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. Regarding the TL1A binding domain, in some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4). Regarding the TL23 binding domain, in some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 binding domain is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the IL23 binding domain is capable of binding human IL23p19 antigen (see SEQ ID NO: 1).

In some embodiments, the VH1 comprises a vhCDR1, a vhCDR2, and a vhCDR3 contained within a variable heavy domain comprising the amino acid sequence of SEQ ID NO: 1674, (ii) the VL1 comprises a vlCDR1, a vlCDR2, and a vlCDR3 contained within a variable light domain comprising the amino acid sequence of SEQ ID NO: 1204, (iii) the VH2 comprises a vhCDR1, a vhCDR2, and a vhCDR3 contained within a variable heavy domain comprising the amino acid sequence of SEQ ID NO: 179, and (iv) the VL2 comprises a vlCDR1, a vlCDR2, and a vlCDR3 contained within a variable light domain comprising the amino acid sequence of SEQ ID NO: 1248. In some embodiments, the CDRs are as defined by Kabat. In other embodiments, the CDRs are as defined by Chothia, Kabat+Chothia, AbM, contact, or IMGT. In some embodiments, the VH1 comprises a vhCDR1 comprising the amino acid sequence of SEQ ID NO: 102, a vhCDR2 comprising the amino acid sequence of SEQ ID NO: 1924, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some embodiments, a binding domain comprises a set of 6 CDRs with 1, 2, 3, 4, 5, 6, 7, 8, 9, 10 amino acid modifications as compared to the 6 CDRs of any of the above binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, a binding domain comprises a set of 6 CDRs that are at least 90, 95, 97, 98 or 99% identical to the 6 CDRs of any of the above binding domains. In some embodiments, the VH1 comprises the amino acid sequence of SEQ ID NO: 1674, (ii) the VL1 comprises the amino acid sequence of SEQ ID NO: 1204, (iii) the VH2 comprises the amino acid sequence of SEQ ID NO: 179, and (iv) the VL2 comprises the amino acid sequence of SEQ ID NO: 1248. In some such antibodies, the variable heavy domain or the variable light domain can comprise variants of any of the above in which an N-terminal glutamine or glutamate is replaced with pyroglutamate. In some embodiments, a binding domain comprises a variant VH domain and/or variant VL domain having 1, 2, 3, 4, 5, 6, 7, 8, 9 or 10 amino acid changes from any of the above VH and VL domains. In some embodiments, a binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. Regarding the TL1A binding domain, in some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4). Regarding the TL23 binding domain, in some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 binding domain is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the IL23 binding domain is capable of binding human IL23p19 antigen (see SEQ ID NO: 1).

In some embodiments, the VH1 comprises a vhCDR1, a vhCDR2, and a vhCDR3 contained within a variable heavy domain comprising the amino acid sequence of SEQ ID NO: 1200, (ii) the VL1 comprises a vlCDR1, a vlCDR2, and a vlCDR3 contained within a variable light domain comprising the amino acid sequence of SEQ ID NO: 1204, (iii) the VH2 comprises a vhCDR1, a vhCDR2, and a vhCDR3 contained within a variable heavy domain comprising the amino acid sequence of SEQ ID NO: 179, and (iv) the VL2 comprises a vlCDR1, a vlCDR2, and a vlCDR3 contained within a variable light domain comprising the amino acid sequence of SEQ ID NO: 1248. In some embodiments, the CDRs are as defined by Kabat. In other embodiments, the CDRs are as defined by Chothia, Kabat+Chothia, AbM, contact, or IMGT. In some embodiments, the VH1 comprises a vhCDR1 comprising the amino acid sequence of SEQ ID NO: 1201, a vhCDR2 comprising the amino acid sequence of SEQ ID NO: 1202, and a vhCDR3 comprising the amino acid sequence of SEQ ID NO: 1203, (ii) the VL1 comprises a vlCDR1 comprising the amino acid sequence of SEQ ID NO: 106, a vlCDR2 comprising the amino acid sequence of SEQ ID NO: 107, and a vlCDR3 comprising the amino acid sequence of SEQ ID NO: 108, (iii) the VH2 comprises a vhCDR1 comprising the amino acid sequence of SEQ ID NO: 180, a vhCDR2 comprising the amino acid sequence of SEQ ID NO: 181, and a vhCDR3 comprising the amino acid sequence of SEQ ID NO: 182, and (iv) the VL2 comprises a vlCDR1 comprising the amino acid sequence of SEQ ID NO: 184, a vlCDR2 comprising the amino acid sequence of SEQ ID NO: 1249, and a vlCDR3 comprising the amino acid sequence of SEQ ID NO: 1250. In some embodiments, a binding domain comprises a set of 6 CDRs with 1, 2, 3, 4, 5, 6, 7, 8, 9, 10 amino acid modifications as compared to the 6 CDRs of any of the above binding domains. That is, the CDRs can be modified as long as the total number of changes in the set of 6 CDRs is less than 11 amino acid modifications, with any combination of CDRs being changed (e.g., there may be one change in vlCDR1, two in vhCDR2, none in vhCDR3, etc.). In some embodiments, a binding domain comprises a set of 6 CDRs that are at least 90, 95, 97, 98 or 99% identical to the 6 CDRs of any of the above binding domains. In some embodiments, the VH1 comprises the amino acid sequence SEQ ID NO: 1200, (ii) the VL1 comprises the amino acid sequence of SEQ ID NO: 1204, (iii) the VH2 comprises the amino acid sequence of SEQ ID NO: 179, and (iv) the VL2 comprises the amino acid sequence of SEQ ID NO: 1248. In some such antibodies, the variable heavy domain or the variable light domain can comprise variants of any of the above in which an N-terminal glutamine or glutamate is replaced with pyroglutamate. In some embodiments, a binding domain comprises a variant VH domain and/or variant VL domain having 1, 2, 3, 4, 5, 6, 7, 8, 9 or 10 amino acid changes from any of the above VH and VL domains. In some embodiments, a binding domain comprises a variant VH domain and/or variant VL domain at least 90%, 95%, 97%, 98%, or 99% identical to any of the above VH and VL domains. In some embodiments, the variant VH domain and/or VL domain has amino acid changes in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. Regarding the TL1A binding domain, in some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. In certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4). Regarding the TL23 binding domain, in some embodiments, the VL sequence includes 66V (i.e., a V at position 66) and/or 105Y (i.e., a Y at position 105) as numbered by Kabat. In some embodiments, the VL sequence includes 66V and 105Y In certain embodiments, the IL23 binding domain is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the IL23 binding domain is capable of binding human IL23p19 antigen (see SEQ ID NO: 1).

In some embodiments, an anti-TL1A×anti-TL23 antibody comprises a first heavy chain, a first light chain, a second heavy chain, and a second light chain. In some embodiments, the two heavy chains and the two light chains comprise the sets of heavy and light chains in XENP53412, XENP53413, XENP53369, XENP53375, XENP53387, XENP53415, XENP53370, XENP53371, XENP53376, XENP53377, XENP53378, XENP53379, XENP53380, or XENP53414. In some embodiments, an anti-TL1A×anti-IL23 antibody comprises: (1) an anti-TL1A heavy chain and light chain pair from XENP53412, XENP53413, XENP53369, XENP53375, XENP53370, XENP53371, XENP53376, XENP53377, XENP53378, XENP53379, XENP53380, or XENP53414, or heavy chain variants thereof in which the N-terminal glutamine is replaced with pyroglutamate and/or the C-terminal lysine or the C-terminal lysine and glycine are removed, or comprises an anti-TL1A heavy chain and light chain pair from XENP53415 or XENP53387 or heavy chain variants thereof in which the C-terminal lysine or the C-terminal lysine and glycine are removed and/or light chain variants thereof in which the N-terminal glutamine is replaced with pyroglutamate; and (2) an anti-IL23 heavy chain and light chain pair from XENP53412, XENP53413, XENP53369, XENP53375, XENP53387, XENP53415, XENP53370, XENP53371, XENP53376, XENP53377, XENP53378, XENP53379, XENP53380, or XENP53414, or heavy chain variants thereof in which the N-terminal glutamine is replaced with pyroglutamate and/or the C-terminal lysine or the C-terminal lysine and glycine are removed. In some embodiments, the first heavy chain comprises the amino acid sequence of SEQ ID NO: 2240 or a variant thereof in which the N-terminal glutamine is replaced with pyroglutamate and/or the C-terminal lysine or the C-terminal lysine and glycine are removed, the first light chain comprises the amino acid sequence of SEQ ID NO: 2242, the second heavy chain comprises the amino acid sequence of SEQ ID NO: 2244 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2245. In some embodiments, the first heavy chain comprises the amino acid sequence of SEQ ID NO: 2240 or a variant thereof in which the N-terminal glutamine is replaced with pyroglutamate and/or the C-terminal lysine or the C-terminal lysine and glycine are removed, the first light chain comprises the amino acid sequence of SEQ ID NO: 2242, the second heavy chain comprises the amino acid sequence of SEQ ID NO: 2250 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2251. In some embodiments, the first heavy chain comprises the amino acid sequence of SEQ ID NO: 2252 or a variant thereof in which the N-terminal glutamine is replaced with pyroglutamate and/or the C-terminal lysine or the C-terminal lysine and glycine are removed, the first light chain comprises the amino acid sequence of SEQ ID NO: 2242, the second heavy chain comprises the amino acid sequence of SEQ ID NO: 2244 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2245. In some embodiments, the first heavy chain comprises the amino acid sequence of SEQ ID NO: 2258 or a variant thereof in which the N-terminal glutamine is replaced with pyroglutamate and/or the C-terminal lysine or the C-terminal lysine and glycine are removed, the first light chain comprises the amino acid sequence of SEQ ID NO: 2242, the second heavy chain comprises the amino acid sequence of SEQ ID NO: 2244 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2245. In some embodiments, the first heavy chain comprises the amino acid sequence of SEQ ID NO: 2264 or a variant thereof in which the C-terminal lysine or the C-terminal lysine and glycine are removed, the first light chain comprises the amino acid sequence of SEQ ID NO: 2265 or a variant thereof in which the N-terminal glutamine is replaced with pyroglutamate, the second heavy chain comprises the amino acid sequence of SEQ ID NO: 2266 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2267. In some embodiments, the first heavy chain comprises the amino acid sequence of SEQ ID NO: 2264 or a variant thereof in which the C-terminal lysine or the C-terminal lysine and glycine are removed, the first light chain comprises the amino acid sequence of SEQ ID NO: 2269 or a variant thereof in which the N-terminal glutamine is replaced with pyroglutamate, the second heavy chain comprises the amino acid sequence of SEQ ID NO: 2266 or a variant thereof in which the N-terminal glutamine is replaced with pyroglutamate and/or the C-terminal lysine or the C-terminal lysine and glycine are removed, and the second light chain comprises the amino acid sequence of SEQ ID NO: 2267. In some embodiments, an anti-TL1A×anti-IL23 antibody comprises a heavy chain and/or a light chain having 1, 2, 3, 4, 5, 6, 7, 8, 9 or 10 amino acid changes from any of the above heavy chains and light chains. In some embodiments, an anti-TL1A×anti-IL23 antibody comprises a heavy chain and/or a light chain at least 90%, 95%, 97%, 98%, or 99% identical to any of the above heavy chains and light chains. In some embodiments, the amino acid changes are in a constant region but not in a variable region. In some embodiments, the amino acid changes are in a constant region or in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. Regarding the TL1A binding domain, in some embodiments, the VH sequence includes 53Y (i.e., a Y at position 53) and/or 100V (i.e., a V at position 100) as numbered by Kabat. In some embodiments, the VH sequence includes 53Y and 100V. In some embodiments, the VL sequence includes 120Q (i.e., a Q at position 120) as numbered by Kabat. Regarding the IL23 binding domain, in some embodiments, the VL sequence includes 66V (i.e., a V at position 66) and/or 105Y (i.e., a Y at position 105) as numbered by Kabat. In some embodiments, the VL sequence includes 66V and 105Y Regarding the TL1A binding domain, in certain embodiments, the TL1A binding domain is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1a ABD is capable of binding human TL1A antigen (see SEQ ID NO: 4). Regarding the TL23 binding domain, in certain embodiments, the IL23 binding domain is capable of binding to IL23, as measured at least one of a Biacore, surface plasmon resonance (SPR) and/or BLI (biolayer interferometry, e.g., Octet assay) assay, with the latter finding particular use in many embodiments. In particular embodiments, the TL23 binding domain is capable of binding human IL23p19 antigen (see SEQ ID NO: 1).

An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric antibody (e.g., bispecific antibodies) are further discussed in detailed below.

### 1. Skew Variants

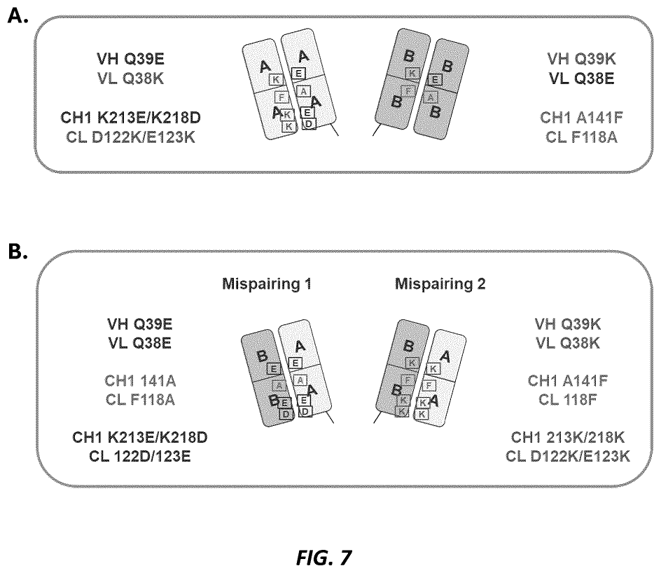

In some embodiments, the heterodimeric antibody includes skew variants which are one or more amino acid modifications in a first heavy chain (A) and first light chain (a) and/or a second heavy chain (B) and second light chain (b) that favor correct heavy chain-light chain pairing (A-a and B-b). In some embodiments, the heterodimeric antibody includes skew variants which are one or more amino acid modifications in a first heavy chain constant domain (A) (e.g., CH1) and first light chain constant domain (a) and/or a second heavy chain constant domain (B) and second light chain constant domain (b) that favor correct heavy chain-light chain pairing (A-a and B-b). In some embodiments, the heterodimeric antibody includes skew variants which are one or more amino acid modifications in a first variable heavy domain (A) and first variable light domain (a) and/or a second variable heavy domain (B) and second variable light domain (b) that favor correct heavy chain-light chain pairing (A-a and B-b). In some embodiments, the heterodimeric antibody includes skew variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in Tables 12-17 and Table 5. Tables 12-17 depict useful pairs of heterodimerization variant sets (including skew and pI variants). In Table 17, there are variants for which there are no corresponding “monomer 2” variants. Such variants are pI variants that can be used alone on either monomer of a bispecific antibody (e.g., anti-TL1A×anti-IL23 bsAb), or included, for example, on the non-scFv side of a format that utilizes an scFv as a component and an appropriate charged scFv linker can be used on the second monomer that utilizes an scFv as the TL1A binding domain. Suitable charged linkers are shown in Table 4. Heterodimer yield (%) and CH3 Tm (° C.) of preferred Fc heterodimerization variants were previously described (see, e.g., FIG. 8 of U.S. Patent Application No. 2019/0248898).

TABLE 12

| Monomer 1 | Monomer 2 |
|---|---|
| F405A | T394F |
| S364D | Y349K |
| S364E | L368K |
| S364E | Y349K |
| S364F | K370G |
| S364H | Y349K |
| S364H | Y349T |
| S364Y | K370G |
| T411K | K370E |
| V397S/F405A | T394F |
| K370R/T411K | K370E/T411E |
| L351E/S364D | Y349K/L351K |
| L351E/S364E | Y349K/L351K |
| L351E/T366D | L351K/T366K |
| P395T/V397S/F405A | T394F |
| S364D/K370G | S364Y/K370R |
| S364D/T394F | Y349K/F405A |
| S364E/F405A | Y349K/T394F |
| S364E/F405S | Y349K/T394Y |
| S364E/T411E | Y349K/D401K |
| S364H/D401K | Y349T/T411E |
| S364H/F405A | Y349T/T394F |
| S364H/T394F | Y349T/F405A |
| Y349C/S364E | Y349K/S354C |
| L351E/S364D/F405A | Y349K/L351K/T394F |
| L351K/S364H/D401K | Y349T/L351E/T411E |
| S364E/T411E/F405A | Y349K/T394F/D401K |
| S364H/D401K/F405A | Y349T/T394F/T411E |
| S364H/F405A/T411E | Y349T/T394F/D401K |

TABLE 13

| Monomer 1 | Monomer 2 |
|---|---|
| K370E/T411D | T411K |
| L368E/K409E | L368K |
| Y349T/T394F/S354C | S364H/F405A/Y349C |
| T411E | D401K |
| T411E | D401R/T411R |
| Q347E/K360E | Q347R |
| L368E | S364K |
| L368E/K370S | S364K |
| L368E/K370T | S364K |
| L368E/D401R | S364K |
| L368E/D401N | S364K |
| L368E | E357S/S364K |
| L368E | S364K/K409E |
| L368E | S364K/K409V |
| L368D | S364K |
| L368D/K370S | S364K |
| L368D/K370S | S364K/E357L |
| L368D/K370S | S364K/E357Q |
| T411E/K360E/Q362E | D401K |
| K370S | S364K |
| L368E/K370S | S364K/E357Q |
| K370S | S364K/E357Q |
| T411E/K360D | D401K |
| T411E/K360E | D401K |
| T411E/Q362E | D401K |
| T411E/N390D | D401K |
| T411E | D401K/Q347K |
| T411E | D401K/Q347R |
| T411E/K360D/Q362E | D401K |

TABLE 14

| Monomer 1 | Monomer 2 |
|---|---|
| T411E/K360E/N390D | D401K |
| T411E/Q362E/N390D | D401K |
| T411E/Q347R | D401K/K360D |
| T411E/Q347R | D401K/K360E |
| T411E/K360 | D401K/Q347K |
| T411E/K360D | D401K/Q347R |
| T411E/K360E | D401K/Q347K |
| T411E/K360E | D401K/Q347R |
| T411E/S364K | D401K/K370S |
| T411E/K370S | D401K/S364K |
| Q347E | E357Q |
| Q347E | E357Q/Q362K |
| K360D/Q362E | Q347R |
| K360D/Q362E | D401K |
| K360D/Q362E | Q347R/D401K |
| K360E/Q362E | Q347R |
| K360E/Q362E | D401K |
| K360E/Q362E | Q347R/D401K |
| Q362E/N390D | D401K |
| Q347E/K360D | D401N |
| K360D | Q347R/N390K |
| K360D | N390K/D401N |
| K360E | Y349H |
| K370S/Q347E | S364K |
| K370S/E357L | S364K |
| K370S/E357Q | S364K |
| K370S/Q347E/E357L | S364K |
| K370S/Q347E/E357Q | S364K |

TABLE 15

| Monomer 1 | Monomer 2 |
|---|---|
| L368D/K370S/Q347E | S364K |
| L368D/K370S/E357L | S364K |
| L368D/K370S/E357Q | S364K |
| L368D/K370S/Q347E/E357L | S364K |
| L368D/K370S/Q347E/E357Q | S364K |
| L368E/K370S/Q347E | S364K |
| L368E/K370S/E357L | S364K |
| L368E/K370S/E357Q | S364K |
| L368E/K370S/Q347E/E357L | S364K |
| L368E/K370S/Q347E/E357Q | S364K |
| L368D/K370T/Q347E | S364K |
| L368D/K370T/E357L | S364K |
| L368D/K370T/E357Q | S364K |
| L368D/K370T/Q347E/E357L | S364K |
| L368D/K370T/Q347E/E357Q | S364K |
| L368E/K370T/Q347E | S364K |
| L368E/K370T/E357L | S364K |
| L368E/K370T/E357Q | S364K |
| L368E/K370T/Q347E/E357L | S364K |
| L368E/K370T/Q347E/E357Q | S364K |
| T411E/Q362E | D401K/T411K |
| T411E/N390D | D401K/T411K |
| T411E/Q362E | D401R/T411R |
| T411E/N390D | D401R/T411R |
| Y407T | T366Y |
| F405A | T394W |
| T366Y/F405A | T394W/Y407T |
| Y407A | T366W |
| T366S/L368A/Y407V | T366W |
| T366S/L368A/Y407V/Y349C | T366W/S354C |

TABLE 16

| Monomer 1 | Monomer 2 |
|---|---|
| T366S/L368A/Y407V/S354C | T366W/Y349C |
| S354C | Y349C |
| K392D/K409D | E356K/D399K |
| K370D/K392D/K409D | E356K/E357K/D399K |
| K274Q/R355Q/N384S/K392N/V397M/Q419E/ | P217R/P228R/N276K |
| K447_{—} | |
| K274Q/R355Q/N384S/K392N/V397M/Q419E/ | N276K |
| K447_{—} | |
| N384S/K392N/V397M/Q419E | N276K |
| D221E/P228E/L368E | D221R/P228R/K409R |
| C220E/P228E/L368E | C220R/E224R/P228R/ |
| | K409R |
| F405L | K409R |
| T366I/K392M/T394W | F405A/Y407V |
| T366V/K409F | L351Y/Y407A |
| T366A/K392E/K409F/T411E | D399R/S400R/Y407A |
| L351K | L351E |
| K274Q/R355Q/Q419E/K447_{—} | P217R/P228R/N276K |
| K274Q/R355Q/Q419E/K447_{—} | N276K |
| I199T/N203D/K274Q/R355Q/N384S/K392N/ | Q196K/I199T/P217R/ |
| V397M/Q419E/K447_{—} | P228R/N276K |
| I199T/N203D/K274Q/R355Q/N384S/K392N/ | Q196K/I199T/N276K |
| V397M/Q419E/K447_{—} | |
| I199T/N203D/K274Q/R355Q/Q419E/K447_{—} | Q196K/I199T/P217R/ |
| | P228R/N276K |
| I199T/N203D/K274Q/R355Q/Q419E/K447_{—} | Q196K/I199T/N276K |

TABLE 17

| | |
|---|---|
| Monomer 1 | Monomer 2 |

I199T/N203D/K274Q/R355Q/N384S/K392N/V397M/
Q419E/K447_{—}
N208D/Q295E/N384D/Q418E/N421D
N208D/Q295E/Q418E/N421D
Q196K/I199T/P217R/P228R/N276K
Q196K/I199T/N276K
K274Q/R355Q/N384S/K392N/V397M/Q419E/K447_{—}
Q295E/N384D/Q418E/N421D
Q295E/Q418E/N421D
P217R/P228R/N276K
N276K
E269Q/E272Q/E283Q/E357Q
E269Q/E272Q/E283Q
E269Q/E272Q
E269Q/E283Q
E272Q/E283Q
E269Q

One particular type of skew variants is generally referred to in the art as “knobs and holes,” referring to amino acid engineering that creates steric influences to favor heterodimeric formation and disfavor homodimeric formation, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometimes referred to herein as “steric variants.” The sequences and tables identify a number of “monomer A-monomer B” pairs that rely on “knobs and holes.” In addition, as described in Merchant et al., Nature Biotech. 16:677 (1998), these “knobs and holes” mutations can be combined with disulfide bonds to further favor formation of Fc heterodimers.

Another method that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs.” In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants.” These include, but are not limited to, D221E/P228E/L368E paired with D221R/P228R/K409R (e.g., these are monomer corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets.” That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization “skew” variants are depicted in Tables 12-17 and 5. Such “skew” variants include, but are not limited to: S364K/E357Q:L368D/K370S; L368D/K370S:S364K; L368E/K370S:S364K; T411T/E360E/Q362E:D401K; L368D/K370S:S364K/E357L; K370S:S364K/E357Q (EU numbering).

In exemplary embodiments, the heterodimeric antibody includes a S364K/E357Q:L368D/K370S; L368D/K370S:S364K; L368E/K370S:S364K; T411T/E360E/Q362E:D401K; L368D/K370S:S364K/E357L; K370S:S364K/E357Q; or a T366S/L368A/Y407V:T366W (optionally including a bridging disulfide, T366S/L368A/Y407V/Y349C:T366W/S354C) “skew” variant amino acid substitution set (EU numbering). In an exemplary embodiment, the heterodimeric antibody includes a “S364K/E357Q:L368D/K370S” amino acid substitution set. In terms of nomenclature, the pair “S364K/E357Q:L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

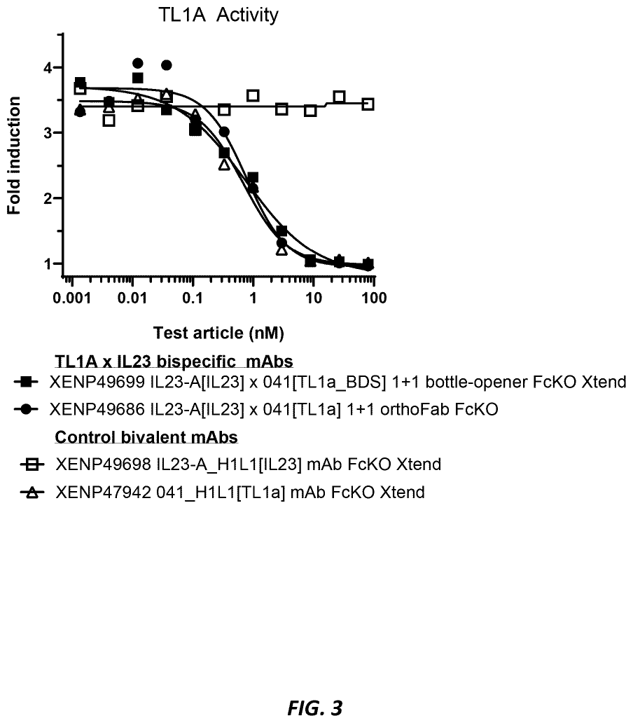

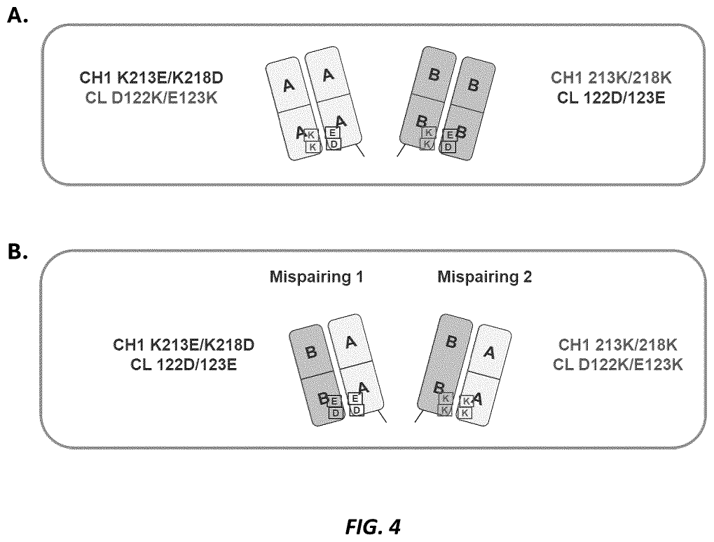

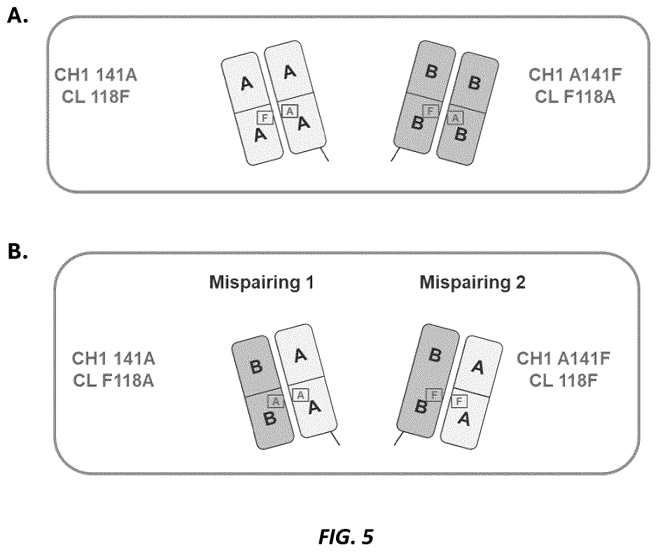

In some embodiments, the subject antibody includes amino acid substitutions at an CH1/CL interface, and/or VH/VL interface that skews correct heavy chain/light chain pairing (see Example 3B). Both electrostatic steering and steric variants can be incorporated in the CH1/CL interface, and/or VH/VL interface to promote correct heavy chain/light chain pairing. Electrostatic steering skew variants at the CH1/CL interface include, for example, substitutions at positions K218 (e.g., K218D) in the CH1 and D122/E123 (e.g., D122K/E123K) in the CL (positions in EU numbering), see FIG. 4 and Table 27. Electrostatic steering skew variants at the CH1/CL interface include, for example, substitutions at positions K213/K218 (e.g., K213E/K218D) in the CH1 and D122/E123 (e.g., D122K/E123K) in the CL (positions in EU numbering), see FIG. 4 and Table 27. Steric skew variants at the CH1/CL interface include, for example, at positions A141 in the CH1 and F116 in the CL; A141 in the CH1 and F118 in the CL (e.g., A141F and F118A); and K147 in CH1 and S131 in the CL (positions in EU numbering), see FIGS. 5 and Table 28. Electrostatic steering skew variants at the VH/VL interface include, for example, those based on a hydrogen bonded pair formed by Q39 in the VH and Q38 in the VL (positions in Kabat numbering), e.g., Q39E:Q38K in VH:VL interface of one Fab of the antibody and Q39K:Q38E in VH:VL interface of the other Fab of the antibody (see FIG. 6). Additional electrostatic steering skew variants at the VH/VL interface are shown in Tables 18 and 19, which depict electrostatic variants which may be introduced at Q39:Q38 in the VH:VL interface. In some embodiments, the subject antibody includes a combination of electrostatic steering and steric variants at the VH/VL and/or CH1/CL interfaces.

TABLE 18

| | |
|---|---|
| Fab A | Fab B |

| VH | VL | VH | VL |
|---|---|---|---|
| Q39E | Q38K | Q39K | Q38E |
| Q39E | Q38K | Q39K | Q38D |
| Q39E | Q38K | Q39R | Q38E |
| Q39E | Q38K | Q39R | Q38D |
| Q39E | Q38K | Q39E | Q38K |
| Q39E | Q38K | Q39E | Q38R |
| Q39E | Q38K | Q39D | Q38K |
| Q39E | Q38K | Q39D | Q38R |
| Q39E | Q38R | Q39K | Q38E |
| Q39E | Q38R | Q39K | Q38D |
| Q39E | Q38R | Q39R | Q38E |
| Q39E | Q38R | Q39R | Q38D |
| Q39E | Q38R | Q39E | Q38K |
| Q39E | Q38R | Q39E | Q38R |
| Q39E | Q38R | Q39D | Q38K |
| Q39E | Q38R | Q39D | Q38R |
| Q39D | Q38K | Q39K | Q38E |
| Q39D | Q38K | Q39K | Q38D |
| Q39D | Q38K | Q39R | Q38E |
| Q39D | Q38K | Q39R | Q38D |
| Q39D | Q38K | Q39E | Q38K |
| Q39D | Q38K | Q39E | Q38R |
| Q39D | Q38K | Q39D | Q38K |
| Q39D | Q38K | Q39D | Q38R |
| Q39D | Q38R | Q39K | Q38E |
| Q39D | Q38R | Q39K | Q38D |
| Q39D | Q38R | Q39R | Q38E |
| Q39D | Q38R | Q39R | Q38D |
| Q39D | Q38R | Q39E | Q38K |
| Q39D | Q38R | Q39E | Q38R |
| Q39D | Q38R | Q39D | Q38K |
| Q39D | Q38R | Q39D | Q38R |
| Q39K | Q38E | Q39K | Q38E |
| Q39K | Q38E | Q39K | Q38D |
| Q39K | Q38E | Q39R | Q38E |
| Q39K | Q38E | Q39R | Q38D |
| Q39K | Q38E | Q39E | Q38K |
| Q39K | Q38E | Q39E | Q38R |
| Q39K | Q38E | Q39D | Q38K |
| Q39K | Q38E | Q39D | Q38R |
| Q39R | Q38E | Q39K | Q38E |

TABLE 19

| | |
|---|---|
| Fab A | Fab B |

| VH | VL | VH | VL |
|---|---|---|---|
| Q39R | Q38E | Q39K | Q38D |
| Q39R | Q38E | Q39R | Q38E |
| Q39R | Q38E | Q39R | Q38D |
| Q39R | Q38E | Q39E | Q38K |
| Q39R | Q38E | Q39E | Q38R |
| Q39R | Q38E | Q39D | Q38K |
| Q39R | Q38E | Q39D | Q38R |
| Q39K | Q38D | Q39K | Q38E |
| Q39K | Q38D | Q39K | Q38D |
| Q39K | Q38D | Q39R | Q38E |
| Q39K | Q38D | Q39R | Q38D |
| Q39K | Q38D | Q39E | Q38K |
| Q39K | Q38D | Q39E | Q38R |
| Q39K | Q38D | Q39D | Q38K |
| Q39K | Q38D | Q39D | Q38R |
| Q39R | Q38D | Q39K | Q38E |
| Q39R | Q38D | Q39K | Q38D |
| Q39R | Q38D | Q39R | Q38E |
| Q39R | Q38D | Q39R | Q38D |
| Q39R | Q38D | Q39E | Q38K |
| Q39R | Q38D | Q39E | Q38R |
| Q39R | Q38D | Q39D | Q38K |
| Q39R | Q38D | Q39D | Q38R |

In some embodiments, the antibody includes Q39E:Q38K mutations in the VH1:VL1 interface of the first Fab. In some embodiments, the antibody includes K213E/K218D:D122K/E123K mutations in the CH1:CL interface of the first Fab. In some embodiments, the antibody includes Q39E:Q38K mutations in the VH1:VL1 interface and K213E/K218D:D122K/E123K mutations in the CH1:CL interface of the first Fab. In some embodiments, the antibody includes Q39K:Q38E mutations in the VH2:VL2 interface of the second Fab. In some embodiments, the antibody includes A141F:F118A mutations in CH1:CL interface of the second Fab. In some embodiments, the antibody includes a Q39K:Q38E mutations in the VH2:VL2 interface and A141F:F118A mutations in the CH1:CL interface of the second Fab. In some embodiments, the antibody includes Q39E:Q38K mutations in the VH1:VL1 interface and K213E/K218D:D122K/E123K mutations in the CH1:CL interface of the first Fab and Q39K:Q38E mutations in the VH2:VL2 interface and A141F:F118A mutations in the CH1:CL interface of the second Fab.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotypic variants, FcRn variants, ablation variants, etc. into one or both of the first and second Fc domains of the heterodimeric antibody. Further, individual modifications can also independently and optionally be included or excluded from the subject the heterodimeric antibody.

In some embodiments, the skew variants outlined herein can be optionally and independently incorporated with any pI variant (or other variants such as Fc variants, FcRn variants, etc.) into one or both heavy chain monomers, and can be independently and optionally included or excluded from the subject heterodimeric antibodies.

### 2. Purification Variants

In some embodiments, the heterodimeric antibody includes purification variants that advantageously allow for the separation of heterodimeric proteins (e.g., anti-TL1A×anti-IL23 bispecific antibody) from homodimeric proteins.

There are several basic mechanisms that can lead to ease of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins. Alternatively, some scaffold formats, such as the “1+1 Fab-scFv-Fc” format, and the “2+1 Fab2-scFv-Fc” format, allows separation on the basis of size. As described above, it is also possible to “skew” the formation of heterodimers over homodimers using skew variants. Thus, a combination of heterodimerization skew variants and purification variants find particular use in the heterodimeric antibodies provided herein.

Additionally, as more fully outlined below, depending on the format of the heterodimeric antibody, purification variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc”, format can include charged scFv linkers (either positive or negative), that give a further pI boost for purification purposes. Some 1+1 Fab-scFv-Fc and 2+1 Fab2-scFv-Fc formats are useful with just charged scFv linkers and no additional pI adjustments, although the invention does provide pI variants that are on one or both of the monomers, and/or charged domain linkers as well. In addition, additional amino acid engineering for alternative functionalities may also confer pI changes, such as Fc, FcRn and KO variants.

In subject heterodimeric antibodies that utilizes pI as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B change be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (aspartic acid to lysine) or changing a charged residue to a neutral residue (e.g., loss of a charge; lysine to serine). A number of these variants are shown in Tables 12-17 and 20.

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention.

The number of pI variants to be included on each or both monomer(s) to get good separation will depend in part on the starting pI of the components, for example in the 1+1 Fab-scFv-Fc, 2+1 Fab2-scFv-Fc, 1+1 CLC and 2+1 CLC formats, the starting pI of the scFv (1+1 Fab-scFv-Fc, 2+1 Fab2-scFv-Fc) and Fab(s) of interest. That is, to determine which monomer to engineer or in which “direction” (e.g., more positive or more negative), the Fv sequences of the two target antigens are calculated and a decision is made from there. Different Fvs will have different starting pIs which are exploited in the present invention. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively, or in addition to isotypic substitutions, the possibility of immunogenicity resulting from the pI variants is significantly reduced by utilizing isosteric substitutions (e.g., Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

Exemplary combinations of pI variants are shown in Tables 12-17 and 20, and FIG. 30 of US Publ. App. No. 2016/0355608, all of which are herein incorporated by reference in its entirety and specifically for the disclosure of pI variants. Preferred combinations of pI variants are shown in Tables 12-17 and 20. As outlined herein and shown in the tables, these changes are shown relative to IgG1, but all isotypes can be altered this way, as well as isotype hybrids. In the case where the heavy chain constant domain is from IgG2-4, R133E and R133Q can also be used.

In one embodiment, one monomer comprises N208D/Q295E/N384D/Q418E/N421D substitutions. In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)₄ (SEQ ID NO: 16). However, the first monomer includes a CH1 domain, including position 208. Accordingly, in constructs that do not include a CH1 domain (for example for antibodies that do not utilize a CH1 domain on one of the domains), a preferred negative pI variant Fc set includes 295E/384D/418E/421D variants (Q295E/N384D/Q418E/N421D when relative to human IgG1).

Accordingly, in some embodiments, one monomer has a set of substitutions from Table 5 and the other monomer has a charged linker (either in the form of a charged scFv linker because that monomer comprises an scFv or a charged domain linker, as the format dictates, which can be selected from those depicted in Table 4).

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., an antibody provided herein may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389,392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

In some embodiments, the anti-TL1A×anti-IL23 antibody includes amino acid substitutions in one of its Fc domains that reduces binding to Protein A. Such purification variants produces heterodimers with asymmetric binding to Protein A, which can in turn be used for separation of heterodimeric from homodimeric populations by a pH gradient. Exemplary purification amino acid substitutions that reduce binding to Protein A include, but are not limited to H435R and Y436F (IgG1 CH3 domain, EU numbering). See, e.g., US2010331527, which is incorporated by reference in its entirety, and specifically for pertinent disclosures relating to Fc domain modifications to reduce Protein A binding.

### 3. Isotypic Variants

In addition, many embodiments of the subject heterodimeric antibodies rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significantly affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

### 4. Calculating pI

The pI of each monomer of the antibodies provided herein can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US Pub. 2014/0370013. As discussed herein, which monomer to engineer is generally decided by the inherent pI of the Fv and scaffold regions. Alternatively, the pI of each monomer can be compared.

5. PI Variants that Also Confer Better FcRn In Vivo Binding

In the case where the pI variant decreases the pI of the monomer, the pI variant can have the added benefit of improving serum retention in vivo.

Although still under examination, Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, ~7.4, induces the release of Fc back into the blood. In mice, Dall' Acqua et al. showed that Fc mutants with increased FcRn binding at pH 6 and pH 7.4 actually had reduced serum concentrations and the same half-life as wild-type Fc (Dall' Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010 PEDS. 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### E. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc., as discussed below.

Accordingly, the anti-TL1A, anti-IL23, and anti-TL1A×anti-IL23 antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

### 1. FcγR and FcRn Variants

Accordingly, there are a number of useful Fc substitutions that can be made to alter binding to one or more of the FcγR receptors. In certain embodiments, the subject antibody includes modifications that alter the binding to one or more FcγR receptors (i.e., “FcγR variants”). Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the subject antibodies include those listed in U.S. Pat. No. 8,188,321 (particularly FIG. 41) and U.S. Pat. No. 8,084,582, and US Publ. App. Nos. 20060235208 and 20070148170, all of which are expressly incorporated herein by reference in their entirety and specifically for the variants disclosed therein that affect Fcγ receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T. Such modification may be included in one or both Fc domains of the subject antibody.

In some embodiments, the subject antibody includes one or more Fc modifications that increase serum half-life. Fc substitutions that find use in increased binding to the FcRn receptor and increased serum half-life, as specifically disclosed in US 2009-0163699 A1, hereby incorporated by reference in its entirety, include, but are not limited to, 434S, 434A, 428L, 308F, 259I, 428L/434S, 259I/308F, 436I/428L, 436I or V/434S, 436V/428L, 259I/308F/428L, and M252Y/S254T/T256E. In some embodiments, M428L/N434S substitutions are used. Such modification may be included in one or both Fc domains of the subject antibody. 2. Ablation Variants

In some embodiments, the subject antibody includes one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, it is generally desirable to ablate FcγRIIIa binding to eliminate or significantly reduce ADCC activity. In some embodiments, of the subject antibodies described herein, at least one of the Fc domains comprises one or more Fcγ receptor ablation variants. In some embodiments, of the subject antibodies described herein, both of the Fc domains comprises one or more Fcγ receptor ablation variants. These ablation variants are depicted in Table 1, and each can be independently and optionally included or excluded, with preferred aspects utilizing ablation variants selected from the group consisting of: L234A/L235A/D265S, G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. In some embodiments, E233P/L234V/L235A/G236del/S267K substitutions are used. It should be noted that the ablation variants referenced herein ablate FcγR binding but generally not FcRn binding.

The Fc domain of human IgG1 has the highest binding to the Fcγ receptors, and thus ablation variants can be used when the constant domain (or Fc domain) in the backbone of the subject antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fc receptors, and thus those backbones can be used with or without the ablation variants.

### F. Combination of Heterodimeric and Fc Variants

All of the recited heterodimerization variants (including skew and/or purification variants) can be optionally and independently combined in any way, as long as they retain their “strandedness” or “monomer partition.” In addition, all of these variants can be combined into any of the heterodimerization formats.

In the case of pI variants, while embodiments finding particular use are shown in the tables, other combinations can be generated, following the basic rule of altering the pI difference between two monomers to facilitate purification.

In addition, any of the heterodimerization variants (skew and purification variants), are also independently and optionally combined with Fc ablation variants, Fc variants, FcRn variants, as generally outlined herein.

Exemplary combination of variants that are included in some embodiments of the heterodimeric 1+1 Fab-scFv-Fc, 2+1 mAb-Fc, 2+1 Fab2-scFv-Fc, and 2+1 Fab2-Fc×scFv-Fc format antibodies are included in Table 5. In some embodiments, the heterodimeric antibody includes a combination of variants as depicted in Table 5.

In some embodiments, the first heavy chain constant domain comprises amino acid substitutions E233P/L234V/L235A/G236del/S267K and/or the second heavy chain constant domain comprises amino acid substitutions E233P/L234V/L235A/G236del/S267K, wherein numbering is according to EU numbering. In some embodiments, the first heavy chain constant domain comprises amino acid substitutions E233P/L234V/L235A/G236del/S267K and the second heavy chain constant domain comprises amino acid substitutions E233P/L234V/L235A/G236del/S267K, wherein numbering is according to EU numbering.

In some embodiments, the first heavy chain constant domain comprises amino acid substitutions M428L/N434S and/or the second heavy chain constant domain comprises amino acid substitutions M428L/N434S, wherein numbering is according to EU numbering. In some embodiments, the first heavy chain constant domain comprises amino acid substitutions M428L/N434S and the second heavy chain constant domain comprises amino acid substitutions M428L/N434S, wherein numbering is according to EU numbering.

In some embodiments, the first heavy chain constant domain comprises amino acid substitutions E233P/L234V/L235A/G236del/S267K and M428L/N434S and/or the second heavy chain constant domain comprises amino acid substitutions E233P/L234V/L235A/G236del/S267K and M428L/N434S, wherein numbering is according to EU numbering. In some embodiments, the first heavy chain constant domain comprises amino acid substitutions E233P/L234V/L235A/G236del/S267K and M428L/N434S and the second heavy chain constant domain comprises amino acid substitutions E233P/L234V/L235A/G236del/S267K and M428L/N434S, wherein numbering is according to EU numbering.

In some embodiments, the first heavy chain constant domain or the second heavy chain constant domain comprises amino acid substitutions L368D/K370S, wherein numbering is according to EU numbering. In some embodiments, the first heavy chain constant domain or the second heavy chain constant domain comprises amino acid substitutions E357Q/S364K, wherein numbering is according to EU numbering. In some embodiments, the first heavy chain constant domain comprises amino acid substitutions L368D/K370S, and the second heavy chain constant domain comprises amino acid substitutions E357Q/S364K, wherein numbering is according to EU numbering.

In some embodiments, the first heavy chain constant domain or the second heavy chain constant domain comprises amino acid substitutions N208D/Q295E/N384D/Q418E/N421D, wherein numbering is according to EU numbering. In some embodiments, the first heavy chain constant domain comprises amino acid substitutions N208D/Q295E/N384D/Q418E/N421D, wherein numbering is according to EU numbering.

In some embodiments, the first heavy chain constant domain or the second heavy chain constant domain comprises amino acid substitutions E233P/L234V/L235A/G236del/S267K, M428L/N434S, L368D/K370S, and N208D/Q295E/N384D/Q418E/N421D, wherein numbering is according to EU numbering. In some embodiments, the first heavy chain constant domain comprises amino acid substitutions E233P/L234V/L235A/G236del/S267K, M428L/N434S, L368D/K370S, and N208D/Q295E/N384D/Q418E/N421D, wherein numbering is according to EU numbering.

In some embodiments, the first heavy chain constant domain or the second heavy chain constant domain comprises amino acid substitutions E233P/L234V/L235A/G236del/S267K, M428L/N434S, and E357Q/S364K, wherein numbering is according to EU numbering. In some embodiments, the second heavy chain constant domain comprises amino acid substitutions E233P/L234V/L235A/G236del/S267K, M428L/N434S, and E357Q/S364K, wherein numbering is according to EU numbering.

In some embodiments, the first heavy chain constant domain comprises amino acid substitutions E233P/L234V/L235A/G236del/S267K, M428L/N434S, L368D/K370S, and N208D/Q295E/N384D/Q418E/N421D, and the second heavy chain constant domain comprises amino acid substitutions E233P/L234V/L235A/G236del/S267K, M428L/N434S, and E357Q/S364K, wherein numbering is according to EU numbering.

### G. Useful Antibody Formats

As discussed more fully below, the heterodimeric bispecific antibodies provided herein can take on several different configurations as generally depicted in FIG. 1.

The heterodimeric formats of the invention can have different valencies as well as be bispecific. That is, heterodimeric antibodies of the invention can be bivalent and bispecific, or trivalent and bispecific, wherein the first antigen is bound by two binding domains and the second antigen by a second binding domain.

The present invention utilizes TL1A binding domains in combination with IL23 binding domains. Any collection of anti-TL1A CDRs, anti-TL1A variable light and variable heavy domains, Fabs and scFvs as depicted in any of the tables (see particularly Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see particularly Tables 7-10) or variants thereof can be used. Similarly, any of the IL23 antigen-binding domains can be used, whether CDRs, variable light and variable heavy domains, Fabs and scFvs as depicted in any of the tables (e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or e.g., Table 11) or variants thereof can be used, optionally and independently combined in any combination.

### 1. 1+1 Fab-scFv-Fc Format

One heterodimeric antibody format that finds particular use in subject anti-TL1A×anti-IL23 antibodies provided herein is the “1+1 Fab-scFv-Fc” or “bottle opener” format as shown in FIG. 1, panel A. The 1+1 Fab-scFv-Fc format antibody includes a first monomer that is a “regular” heavy chain (VH1-CH1-hinge-CH2-CH3), wherein VH1 is a first variable heavy domain and CH2-CH3 is a first Fc domain. The 1+1 Fab-scFv-Fc also includes a light chain that includes a first variable light domain VL1 and a constant light domain CL. The light chain interacts with the VH1-CH1 of the first monomer to form a first antigen-binding domain that is a Fab. The second monomer of the antibody includes a second binding domain that is a single chain Fv (“scFv,” as defined below) and a second Fc domain. The scFv includes a second variable heavy domain (VH2) and a second variable light domain (VL2), wherein the VH2 is attached to the VL2 using an scFv linker that can be charged (see, e.g., Table 4). The scFv is attached to the heavy chain using a domain linker (see, e.g., Table 6). The two monomers are brought together by the use of amino acid variants (e.g., heterodimerization variants, discussed above) in the constant regions (e.g., the Fc domain, the CH1 domain and/or the hinge region) that promote the formation of heterodimeric antibodies as is described more fully below. This structure is sometimes referred to herein as the “bottle-opener” format, due to a rough visual similarity to a bottle-opener. In some embodiments, the 1+1 Fab-scFv-Fc format antibody is a bivalent antibody.

There are several distinct advantages to the present “1+1 Fab-scFv-Fc” format. Antibody analogs relying on two scFv constructs often have stability and aggregation problems, which can be alleviated in the present invention by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

In some embodiments of the 1+1 Fab-scFv-Fc format antibody, one of the first or second antigen-binding domain is a TL1A binding domain and the other binding domain is a IL23 binding domain. In some embodiments where the 1+1 Fab-scFv-Fc, it is the scFv that binds to the TL1A, and the Fab that binds IL23. Exemplary anti-TL1A×anti-IL23 bispecific antibody in the 1+1 Fab-scFv-Fc format are depicted in Tables 26 and 31 (affinity optimized).

In some embodiments, the first and second Fe domains of the 1+1 Fab-scFv-Fc format antibody are variant Fe domains that include heterodimerization skew variants (e.g., a set of amino acid substitutions as shown in Tables 12-17 and 5). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fe domains includes heterodimerization skew variants L368D/K370S and the other of the first or second variant Fe domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fe domains include ablation variants (including those shown in Table 1). In some embodiments, each of the first and second variant Fe domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in Tables 12-17 and 20). In exemplary embodiments, the constant domain (CH1-hinge-CH2-CH3) of the first monomer includes pI variants N208D/Q295E/N384D/Q418E/N421D, wherein numbering is according to EU numbering.

In exemplary embodiments, the 1+1 Fab-scFv-Fc format antibody includes a combination of amino acid modifications as depicted in Table 5. In such embodiments, the CH1-hinge-CH2-CH3 of the first monomer comprises amino acid variants L368D/K370S/N208D/Q295E/N384D/Q418E/N421D/E233P/L234V/L235A/G236del/S267 K, the second Fc domain comprises amino acid variants S364K/E357Q/E233P/L234V/L235A/G236del/S267K, wherein numbering is according to EU numbering.

In some embodiments, the scFv of the 1+1 Fab-scFv-Fc format antibody provided herein includes a charged scFv linker (including those shown in Table 4). In some embodiments, the 1+1 Fab-scFv-Fc format antibody provided herein includes FcRn variants M428L/N434S, wherein numbering is according to EU numbering.

In exemplary embodiments 1+1 Fab-scFv-Fc format antibody, the first Fc domain includes heterodimerization skew variants L368D/K370S and the second Fc domain includes heterodimerization skew variants S364K/E357Q; each of the first and second Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 1+1 Fab-scFv-Fc format antibody provided herein includes a (GKPGS)₄ (SEQ ID NO: 16) charged scFv linker. In some embodiments, the 1+1 Fab-scFv-Fc format antibody provided herein includes FcRn variants M428L/N434S, wherein numbering is according to EU numbering. In some embodiments, the scFv of the 1+1 Fab-scFv-Fc format antibody provided herein includes a charged scFv linker (including those shown in Table 4).

Any suitable TL1A binding domain can be included in subject 1+1 Fab-scFv-Fc format antibody, including any of the TL1A binding domains provided herein (see e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23). In some embodiments, the TL1A binding domain is one of the following TL1a binding domains or a variant thereof: 70416_041[TL1a]_H1L1, 70417_111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (Tables 7-10).

Any suitable IL23 binding domain can be included in subject 1+1 Fab-scFv-Fc format antibody, including any of the IL23 binding domains provided herein (see e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., SEQ ID NOS: 90-95 and FIG. 5). In some embodiments, the IL23 binding domain is IL23-A[IL23]_H1L1 (Table 11) or a variant thereof.

Exemplary Fc domain sequences that are useful in the 1+1 Fab-scFv-Fc format antibodies are provided in SEQ ID NOS: 58-87 (corresponding SEQ ID NOS for each heterodimeric Fc backbone are provided in the Brief Description of Sequences). These are heterodimeric anti-TL1A×anti-IL23 bsAb backbones based on human IgG1, without the cytokine sequences. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants on both chains. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297S variant that removes glycosylation on both chains. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants on both chains. Heterodimeric Fc backbone 13 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 14 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 15 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” sequence, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of the sequence. Additionally, the backbones depicted herein may include deletion of the C-terminal glycine (G446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

Exemplary CH1-hinge domains, CH1 domains, and hinge domains that can be included in the first or second monomer of the 1+1 Fab-scFv-Fc format are provided in SEQ ID NOS: 90-98. Further, useful CL sequences that can be used with this format, i.e., constant domain of the cognate light chains that find use in the subject anti-TL1A×anti-IL23 bsAbs that utilize a Fab binding domain, are provided in SEQ ID NOS: 99 and 100.

Exemplary anti-TL1A×anti-IL23 bispecific antibodies in the 1+1 Fab-scFv-Fc format are depicted in Table 26. In certain embodiments, a bispecific antibody comprises any of the antibodies depicted in Table 26 or a variant thereof. In some embodiments, an anti-TL1A×anti-IL23 antibody comprises a heavy chain and/or a light chain having 1, 2, 3, 4, 5, 6, 7, 8, 9 or 10 amino acid changes from any of the above heavy chains and light chains. In some embodiments, an anti-TL1A×anti-IL23 antibody comprises a heavy chain and/or a light chain at least 90%, 95%, 97%, 98%, or 99% identical to any of the above heavy chains and light chains. In some embodiments, the amino acid changes are in a constant region but not in a variable region. In some embodiments, the amino acid changes are in a constant region or in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. Regarding the TL1A binding domain, in some embodiments, the VH sequence includes 53Y (i.e., a Y at position 53) and/or 100V (i.e., a V at position 100) as numbered by Kabat.

### 2. OrthoFab

One heterodimeric antibody format that finds particular use in subject anti-TL1A×anti-IL23 antibodies provided herein is the “OrthoFab” format that facilitates correct VH/VL pairings. In this format, the antibody is an IgG format antibody with two heavy chains and two light chains. The first heavy chain is according to VH1-CH1-hinge-CH2-CH3, and the second heavy chain is VH2-CH1-hinge-CH2-CH3, wherein VH1 is a first variable heavy domain, VH2 is a second variable heavy domain, CH2-CH3 of the first heavy chain is a first Fc domain, and CH2-CH3 of the second heavy chain is a second Fc domain. The first light chain is VL1-CL, and the second light chain is VL2-CL, wherein VL1 is a first variable light domain, and VL2 is a second variable light domain. The VH1 and VL1 form a first antigen-binding domain, and the VH2 and VL2 form a second antigen-binding domain, where one of the first and second antigen-binding domains is the TL1A binding domain, and the other of the first and second antigen-binding domains is the IL23 binding domain.

The OrthoFab format further includes amino acid substitutions at one or more CH1/CL interfaces, and/or VH/VL interfaces that skews correct heavy chain/light chain pairing (see Example 3B). Both electrostatic steering and steric variants can be incorporated in the CH1/CL interface and/or VH/VL interface to promote correct heavy chain/light chain pairing.

In some embodiments, the antibody includes electrostatic steering skew variants at an CH1/CL interface. In some embodiments, the antibody includes a CH1/CL with electrostatic steering skew variants selected from variants depicted in FIG. 4 or Table 27. In some embodiments, the antibody includes a CH1/CL with steric skew variants selected from variants depicted in FIG. 5 or 6. In some embodiments, the antibody includes a VH/VL with electrostatic steering skew variants selected from variants depicted in FIG. 6 or Tables 18 and 19.

In some embodiments, the antibody includes a combination of one or more of the CH1/CL electrostatic steering and/or steric skew variants, and VH/VL electrostatic steering skew variants depicted in FIGS. 4-7 and Tables 27-28 and 18-19. In some embodiments, the first heavy chain and first light chain each include a) a CH1/CL with electrostatic steering and/or steric skew variants and b) a VH1/VL1 with electrostatic steering variants. In some embodiments, the second heavy chain and second light chain further includes a) a CH1/CL with electrostatic steering and/or steric skew variants and b) a VH2/VL2 with electrostatic steering variants. In some embodiments, the antibody includes Q39E:Q38K mutations in the VH1:VL1 interface of the first Fab. In some embodiments, the antibody includes K213E/K218D:D122K/E123K mutations in the CH1:CL interface of the first Fab. In some embodiments, the antibody includes Q39E:Q38K mutations in the VH1:VL1 interface and K213E/K218D:D122K/E123K mutations in the CH1:CL interface of the first Fab. In some embodiments, the antibody includes Q39K:Q38E mutations in the VH2:VL2 interface of the second Fab. In some embodiments, the antibody includes A141F:F118A mutations in CH1:CL interface of the second Fab. In some embodiments, the antibody includes a Q39K:Q38E mutations in the VH2:VL2 interface and A141F:F118A mutations in the CH1:CL interface of the second Fab. In some embodiments, the antibody includes Q39E:Q38K mutations in the VH1:VL1 interface and K213E/K218D:D122K/E123K mutations in the CH1:CL interface of the first Fab and Q39K:Q38E mutations in the VH2:VL2 interface and A141F:F118A mutations in the CH1:CL interface of the second Fab.

In some embodiments, the first and second Fc domains of the OrthoFab format are variant Fc domains that include heterodimerization skew variants (e.g., a set of amino acid substitutions as shown in Tables 12-17 and 5). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering. In some embodiments, the antibody includes Q39E:Q38K mutations in the VH1:VL1 interface and K213E/K218D:D122K/E123K mutations in the CH1:CL interface of the first Fab and Q39K:Q38E mutations in the VH2:VL2 interface and A141F:F118A mutations in the CH1:CL interface of the second Fab, and the first Fc domain includes heterodimerization skew variants L368D/K370S and the second Fc domain includes heterodimerization skew variants S364K/E357Q.

In some embodiments, the variant Fc domains include ablation variants (including those shown in Table 1). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in Table 20). Table 20 depicts a list of isosteric variant antibody constant regions and their respective substitutions. pI_(−) indicates lower pI variants, while pI_(+) indicates higher pI variants. These variants can be optionally and independently combined with other variants, including heterodimerization variants, outlined herein. In exemplary embodiments, the constant domain (CH1-hinge-CH2-CH3) of the first or second monomer includes pI variants N208D/Q295E/N384D/Q418E/N421D, wherein numbering is according to EU numbering. In some embodiments, the constant domain of the first monomer includes pI variants N208D/Q295E/N384D/Q418E/N421D.

TABLE 20

| Variant constant region | Substitutions |
|---|---|
| pI-ISO(−) | I199T/N203D/K274Q/R355Q/N384S/ |
| | K392N/N397M/Q418E/K447_{—} |
| pI_ISO(−)-Fc only | K274Q/R355Q/N384S/K392N/ |
| | V397M/Q418E/K447_{—} |
| pI_(−)_isosteric_A | N208D/Q295E/N384D/Q418E/ |
| | N421D |
| pI_(−)_isosteric A-Fc only | Q295E/N384D/Q418E/N421D |
| pI_(−)_isosteric_B | N208D/Q295E/Q418E/N421D |
| pI_(−)_isosteric_B-Fc only | Q295E/Q418E/N421D |
| pI_ISO(+RR) | Q196K/I199T/P217R/P228R/ |
| | N276K |
| pI_ISO(+RR)-Fc only | P217R/P228R/N276K |
| pI_ISO(+) | Q196K/I199T/N276K |
| pI_ISO(+)-Fc only | N276K |
| pI_(+)_isosteric_A | E269Q/E272Q/E283Q/E357Q |
| pI_(+)_isosteric_B | E269Q/E272Q/E283Q |
| pI_(+)_isosteric_E269Q/E272Q | E269Q/E272Q |
| pI_(+)_isosteric_E269Q/E283Q | E269Q/E283Q |
| pI_(+)_isosteric_E272Q/E283Q | E272Q/E283Q |
| pI_(+)_isosteric_E269Q | E269Q |

In some embodiments, the OrthoFab format antibody provided herein includes FcRn variants M428L/N434S, wherein numbering is according to EU numbering. In some embodiments, each of the first and second variant Fc domains includes FcRn variants M428L/N434S.

In some embodiments, (i) the antibody includes Q39E:Q38K mutations in the VH1:VL1 interface and K213E/K218D:D122K/E123K mutations in the CH1:CL interface of the first Fab and Q39K:Q38E mutations in the VH12:VL2 interface and A141F:F118A mutations in the CH1:CL interface of the second Fab, (ii) the first Fc domain includes heterodimerization skew variants L368D/K370S and the second Fc domain includes heterodimerization skew variants S364K/E357Q, (iii) each of the first and second Fc domains includes ablation variants E233P/L234V/L235A/G236_/S267K, (iv) each of the first and second Fe domains includes FcRn variants M428L/N434S, and (v) the first heavy chain includes pI variants N208D/Q295E/N384D/Q418E/N421D.

Any suitable TL1A binding domain and TL23 domain can be included in subject OrthoFab format antibody, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and IL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

Exemplary anti-TL1A×anti-IL23 bispecific antibodies in the OrthoFab format are depicted in Tables 29 and 30. Other exemplary anti-TL1A×anti-IL23 bispecific antibodies in the OrthoFab format are depicted in Table 44. In certain embodiments, a bispecific antibody comprises any of the antibodies depicted in Table 55, 29, or 30 or a variant thereof. In some embodiments, an anti-TL1A×anti-TL23 antibody comprises a heavy chain and/or a light chain having 1, 2, 3, 4, 5, 6, 7, 8, 9 or 10 amino acid changes from any of the above heavy chains and light chains. In some embodiments, an anti-TL1A×anti-IL23 antibody comprises a heavy chain and/or a light chain at least 90%, 95%, 97%, 98%, or 99% identical to any of the above heavy chains and light chains. In some embodiments, the amino acid changes are in a constant region but not in a variable region. In some embodiments, the amino acid changes are in a constant region or in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. Regarding the TL1A binding domain, in some embodiments, the VH sequence includes 53Y (i.e., a Y at position 53) and/or 100V (i.e., a V at position 100) as numbered by Kabat.

### 3. CrossMab-VH-VL Charge-Swap 1+1 Format

One heterodimeric antibody format that finds particular use in subject anti-TL1A×anti-IL23 antibodies provided herein is the “CrossMab-VH-VL charge-swap 1+1” format (see Table 32). The CrossMab-VH-VL charge-swap 1+1 format is a Fab×Fab four chain bispecific antibody. In some embodiments, the first monomer of this format includes a first variable light domain (VL1) attached to a heavy chain constant domain comprising a CH1-hinge-CH2-CH3, wherein CH2-CH3 is a first Fc domain. In some embodiments, the second monomer of this format includes a first variable heavy domain (VH1) attached to a constant light domain (e.g., a CLκ). The third monomer comprises a canonical heavy chain that includes a VH2-CH1-hinge-CH2-CH3, wherein VH2 is a second variable heavy domain and CH2-CH3 is a second Fc domain. The fourth monomer comprises a canonical light chain that includes a VL2-CL, wherein VL2 is a second variable light domain and CL is a constant light domain (e.g., a CLκ). The first monomer and second monomer form a “crossed” arm, wherein VL1 and VH1 form a first antigen-binding domain that binds a first antigen, and the third monomer and forth monomer form a “non-crossed” arm, wherein VL2 and VH2 form a second antigen-binding domain that binds a second antigen.

In some embodiments, the heavy chain constant domain (e.g., CH1) of the third monomer and the light chain constant domain (e.g., CLκ) of the fourth monomer further include “charge” swap amino acid substitutions. In some embodiments, these “charge” swap substitutions comprise amino acid substitutions K213E/K218D (EU numbering) in the heavy chain constant domain of the third monomer, and the fourth monomer CL is a CLκ that comprises amino acid substitutions D112K/E123K (Kabat numbering).

Without being bound by any particular theory of operation, it is believed that the crossing of one arm (i.e., the “crossed” arm) and charge swap engineering on the “non-crossed”arm in this format greatly diminishes the mispairing of LCs without affecting the various other developability and biophysical parameters that might be used in development of a biological therapeutic.

In some embodiments, the constant light domain of the second monomer includes amino acid substitutions R108A/T109S (Kabat numbering). In some embodiments, the VL1 of the first monomer is attached to the CH1 of the first monomer by an SS linker. In some embodiments, the “crossed” arm includes isosteric variants N208D/Q295E/N384D/Q418E/N421D in the heavy chain constant domain of the first monomer, wherein numbering is according to EU numbering.

In some embodiments, the first and second Fc domains of the CrossMab-VH-VL charge-swap 1+1 format are variant Fc domains that include heterodimerization skew variants (e.g., a set of amino acid substitutions as shown in Tables 12-17 and 5). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in Table 1). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in Table 20). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the CrossMab-VH-VL charge-swap 1+1 format antibody provided herein includes FcRn variants M428L/N434S, wherein numbering is according to EU numbering. In some embodiments, each of the first and second variant Fc domains includes FcRn variants M428L/N434S.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267 K, and the second Fc domain comprises amino acid variants S364K/E357Q/E233P/L234V/L235A/G236del/S267K, wherein numbering is according to EU numbering.

In some embodiments, the CrossMab-VH-VL charge-swap 1+1 format antibody provided herein further includes FcRn variants M428L/N434S, wherein numbering is according to EU numbering. In some embodiments, (i) amino acid substitutions K213E/K218D (EU numbering) in the heavy chain constant domain of the third monomer, and the fourth monomer CL is a CLκ that comprises amino acid substitutions D112K/E123K (Kabat numbering), (ii) the first Fc domain includes heterodimerization skew variants L368D/K370S and the second Fc domain includes heterodimerization skew variants S364K/E357Q, (iii) each of the first and second Fc domains includes ablation variants E233P/L234V/L235A/G236_/S267K, (iv) each of the first and second Fc domains includes FcRn variants M428L/N434S, and (v) the first heavy chain constant domain includes pI variants N208D/Q295E/N384D/Q418E/N421D.

In some embodiments, one of the first binding domain or the second binding domain binds TL1A and the other binding domain binds IL23. Any suitable TL1A binding domain and IL23 domain can be included in subject CrossMab-VH-VL charge-swap 1+1 format antibody, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and IL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

Exemplary anti-TL1A×anti-IL23 bispecific antibodies in the CrossMab 1+1 format are depicted in Table 31. Exemplary anti-TL1A×anti-IL23 bispecific antibodies in the CrossMab-VH-VL charge-swap 1+1 format are depicted in Table 32. Other exemplary anti-TL1A×anti-IL23 bispecific antibodies in the CrossMab-VH-VL charge-swap 1+1 format are depicted in Table 44. Other exemplary anti-TL1A×anti-IL23 bispecific antibodies in a CrossMab format are depicted in Table 31. In certain embodiments, a bispecific antibody comprises any of the antibodies depicted in Table 44, 32, or 31 or a variant thereof. In some embodiments, an anti-TL1A×anti-IL23 antibody comprises a heavy chain and/or a light chain having 1, 2, 3, 4, 5, 6, 7, 8, 9 or 10 amino acid changes from any of the above heavy chains and light chains. In some embodiments, an anti-TL1A×anti-IL23 antibody comprises a heavy chain and/or a light chain at least 90%, 95%, 97%, 98%, or 99% identical to any of the above heavy chains and light chains. In some embodiments, the amino acid changes are in a constant region but not in a variable region. In some embodiments, the amino acid changes are in a constant region or in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. Regarding the TL1A binding domain, in some embodiments, the VH sequence includes 53Y (i.e., a Y at position 53) and/or 100V (i.e., a V at position 100) as numbered by Kabat.

### 4. 1+1 CLC Format

One heterodimeric antibody format that finds particular use in subject anti-TL1A×anti-IL23 antibodies provided herein is the “1+1 Common Light Chain” or “1+1 CLC” format, which is depicted in FIG. 1, Panel C. The 1+1 CLC format antibody includes a first monomer that includes a VH1-CH1-hinge-CH2-CH3, wherein VH1 is a first variable heavy domain and CH2-CH3 is a first Fc domain; a second monomer that includes a VH2-CH1-hinge-CH2-CH3, wherein VH2 is a second variable heavy domain and CH2-C3 is a second Fc domain; and a third monomer “common light chain” comprising VL-CL, wherein VL is a common variable light domain and CL is a constant light domain. In such embodiments, the VL pairs with the VH1 to form a first binding domain with a first antigen-binding specificity; and the VL pairs with the VH2 to form a second binding domain with a second antigen-binding specificity. In some embodiments, the 1+1 CLC format antibody is a bivalent antibody.

In some embodiments, the first and second Fc domains of the 1+1 CLC format are variant Fc domains that include heterodimerization skew variants (e.g., a set of amino acid substitutions as shown in Tables 12-17 and 5). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fe domains include ablation variants (including those shown in Table 1). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in Table 20). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 1+1 CLC format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267 K, and the second Fc domain comprises amino acid variants S364K/E357Q/E233P/L234V/L235A/G236del/S267K, wherein numbering is according to EU numbering.

In some embodiments, the 1+1 CLC format antibody provided herein further includes FcRn variants M428L/N434S, wherein numbering is according to EU numbering.

In some embodiments, one of the first binding domain or the second binding domain binds TL1A and the other binding domain binds IL23. Any suitable TL1A binding domain and IL23 domain can be included in subject 1+1 CLC format antibody, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and TL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

Exemplary anti-TL1A×anti-IL23 bispecific antibodies in the 1+1 CLC format are depicted in Table 33. In certain embodiments, a bispecific antibody comprises any of the antibodies depicted in Table 33 or a variant thereof. In some embodiments, an anti-TL1A×anti-IL23 antibody comprises a heavy chain and/or a light chain having 1, 2, 3, 4, 5, 6, 7, 8, 9 or 10 amino acid changes from any of the above heavy chains and light chains. In some embodiments, an anti-TL1A×anti-IL23 antibody comprises a heavy chain and/or a light chain at least 90%, 95%, 97%, 98%, or 99% identical to any of the above heavy chains and light chains. In some embodiments, the amino acid changes are in a constant region but not in a variable region. In some embodiments, the amino acid changes are in a constant region or in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. Regarding the TL1A binding domain, in some embodiments, the VH sequence includes 53Y (i.e., a Y at position 53) and/or 100V (i.e., a V at position 100) as numbered by Kabat.

5. 2+2 mAb-scFv

One heterodimeric antibody format that finds particular use in the subject bispecific anti-TL1A×anti-IL23 antibodies is the 2+2 mAb-scFv format shown in FIG. 1, Panel O. This antibody format includes four antigen-binding domains: two Fab portions and two scFvs that are attached to the C-terminal of each of the heavy chain monomers. In some embodiments of this format, the Fab portions each bind IL23, and the “extra” scFv domain binds TL1A. That is, this mAb-scFv format is a trivalent antibody.

In these embodiments, the first monomer comprises, from N- to C-terminal, VH1-CH1-hinge-CH2-CH3-domain linker-scFv, and the second monomer comprises, from N- to C-terminal, VH1-CH1-hinge-CH2-CH3-domain linker-scFv domain, where the scFv domain comprises a second VH (VH2), a second VL (VL2) and a scFv linker. As for all the scFv domains herein, the scFv domains of the 2+2 mAb-scFv can be in either orientation, from N- to C-terminal, VH2-scFv linker-VL2 or VL2-scFv linker-VH2. Accordingly, the first and second monomers may each comprise, from N- to C-terminal, VH1-CH1-hinge-CH2-CH3-domain linker-VH2-scFv linker-VL2 or VH1-CH1-hinge-CH2-CH3-domain linker-VL2-scFv linker-VH2. The composition also comprises a light chain, VL1-CL. In embodiments, this format includes two identical light chains (VL1-CL). In this format, the VH1s are each a first variable heavy (VH) domain, the VL1s are each a first variable light (VL) domain, the VH2 is a second variable heavy domain, and the VL2 is a second variable light domain. In some embodiments, the format includes two identical common light chains (each VL1-CL), wherein each of the common light chains associates with one of the VH1-CH1 of the first and second monomers to form two identical Fabs (i.e., first antigen-binding domains). In some embodiments, the scFvs are the second antigen-binding domains. In some embodiments, the first ABDs bind to human IL23, and the second ABD binds human TL1A.

In some embodiments, the first and second Fc domains of the 2+2 mAb-scFv format antibody are variant Fc domains that include heterodimerization skew variants (e.g., a set of amino acid substitutions as shown in Tables 12-17 and 5). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fe domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in Table 1). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in Tables 12-17 and 20). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+2 mAb-scFv format antibody provided herein includes a charged scFv linker (including those shown in Table 4). In some embodiments, the 2+2 mAb-scFv format antibody provided herein includes FcRn variants M428L/N434S, wherein numbering is according to EU numbering.

In exemplary embodiments the 2+2 mAb-scFv format antibody includes a combination of amino acid modifications as depicted in Table 5. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFvs of the 2+2 mAb-scFv format antibody provided herein include a (GKPGS)₄ (SEQ ID NO: 16) charged scFv linker. In some embodiments, 2+2 mAb-scFv format antibody provided herein includes FcRn variants M428L/N434S, wherein numbering is according to EU numbering.

In some embodiments, the scFvs of the 2+2 mAb-scFv-Fc format antibody are each TL1A binding domains and the VH1 of the first and second monomer and the VL1 of the common light chain each form binding domains that bind IL23. In some embodiments, the scFvs of the 2+2 mAb-scFv-Fc format antibody are each IL23 binding domains and the VH1 of the first and second monomer and the VL1 of the common light chain each form binding domains that bind TL1A.

Any suitable TL1A binding domain and IL23 domain can be included in subject 2+2 mAb-scFv-Fc format antibody, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and IL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

Exemplary anti-TL1A×anti-IL23 bispecific antibodies in the 2+2 mAb-scFv-Fc format and other formats are depicted in Table 34. In certain embodiments, a bispecific antibody comprises any of the antibodies depicted in Table 34 or a variant thereof. In some embodiments, an anti-TL1A×anti-IL23 antibody comprises a heavy chain and/or a light chain having 1, 2, 3, 4, 5, 6, 7, 8, 9 or 10 amino acid changes from any of the above heavy chains and light chains. In some embodiments, an anti-TL1A×anti-IL23 antibody comprises a heavy chain and/or a light chain at least 90%, 95%, 97%, 98%, or 99% identical to any of the above heavy chains and light chains. In some embodiments, the amino acid changes are in a constant region but not in a variable region. In some embodiments, the amino acid changes are in a constant region or in a framework region (e.g., FR1, FR2, FR3, or FR4) but not in a CDR. In some embodiments, the changes are amino acid substitutions at one, two, three, or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, the substitutions are residues that are adjacent to a CDR, residues capable of interacting with the antigen, or residues capable of interacting with the CDR. Regarding the TL1A binding domain, in some embodiments, the VH sequence includes 53Y (i.e., a Y at position 53) and/or 100V (i.e., a V at position 100) as numbered by Kabat.

6. 2+1 mAb-scFv Format

One heterodimeric antibody format that finds particular use in the subject bispecific anti-TL1A×anti-IL23 antibodies is the 2+1 mAb-scFv format shown in FIG. 1, Panel E. This antibody format includes three antigen-binding domains: two Fab portions and an scFv that is attached to the C-terminal of one of the heavy chains. In some embodiments of this format, the Fab portions each bind IL23, in this case, human IL23 and the “extra” scFv domain binds TL1A. That is, this 2+1 mAb-scFv format is a trivalent antibody.

In these embodiments, the first chain or monomer comprises, from N- to C-terminal, VH1-CH1-hinge-CH2-CH3, the second monomer comprises, from N- to C-terminal, VH1-CH1-hinge-CH2-CH3-domain linker-scFv domain, where the scFv domain comprises a second VH (VH2), a second VL (VL2) and a scFv linker. As for all the scFv domains herein, the scFv domain can be in either orientation, from N- to C-terminal, VH2-scFv linker-VL2 or VL2-scFv linker-VH2. Accordingly, the second monomer may comprise, from N- to C-terminal, VH1-CH1-hinge-CH2-CH3-domain linker-VH2-scFv linker-VL2 or VH1-CH1-hinge-CH2-CH3-domain linker-VL2-scFv linker-VH2. The composition also comprises a light chain, VL1-CL. In embodiments, this format includes two identical light chains (VL1-CL). In this format, the VH1s are each a first variable heavy (VH) domain, the VL1s are each a first variable light (VL) domain, the VH2 is a second variable heavy domain, and the VL2 is a second variable light domain. In some embodiments, the format includes two identical common light chains (each VL1-CL), wherein each of the common light chains associates with one of the VH1-CH1 of the first and second monomers to form two identical Fabs (i.e., first antigen-binding domains). In some embodiments, the scFv is the second antigen-binding domain. In some embodiments, the first ABDs bind to human IL23, and the second ABD binds human TL1A. In some embodiments, the first ABDs bind to human TL1A, and the second ABD binds human IL23.

In some embodiments, the first and second Fe domains of the 2+1 mAb-scFv format antibody are variant Fe domains that include heterodimerization skew variants (e.g., a set of amino acid substitutions as shown in Tables 12-17 and 5). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in Table 1). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in Tables 12-17 and 20). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 mAb-scFv format antibody provided herein includes a charged scFv linker (including those shown in Table 4). In some embodiments, the 2+1 mAb-scFv format antibody provided herein includes FcRn variants M428L/N434S, wherein numbering is according to EU numbering.

In exemplary embodiments the 2+1 mAb-scFv format antibody includes a combination of amino acid modifications as depicted in Table 5. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 mAb-scFv format antibody provided herein includes a (GKPGS)₄ (SEQ ID NO: 16) charged scFv linker. In some embodiments, 2+1 mAb-scFv format antibody provided herein includes FcRn variants M428L/N434S, wherein numbering is according to EU numbering.

In some embodiments, the scFv of the second monomer of the 2+1 mAb-scFv-Fc format antibody is a TL1A binding and the VH1 of the first and second monomer and the VL1 of the common light chain each form binding domains that bind IL23. In some embodiments, one of the first binding domain or the second binding domain binds TL1A and the other binding domain binds IL23. In some embodiments, the scFv of the second monomer of the 2+1 mAb-scFv-Fc format antibody is a IL23 binding and the VH1 of the first and second monomer and the VL1 of the common light chain each form binding domains that bind TL1A.

Any suitable TL1A binding domain and TL23 domain can be included in subject 2+1 mAb-scFv-Fc format antibody, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and IL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

Illustrative sequences of heterodimeric anti-TL1A×anti-IL23 bsAb backbone for use in the 2+1 mAb-scFv format are provided in SEQ ID NOS: 88 and 89. The format depicted here is based on heterodimeric Fc backbone 1 set forth in SEQ ID NOS: 58 and 59, except further including G446_on monomer 1 (−) and G446_/K447_on monomer 2 (+). It should be noted that any of the additional backbones in SEQ ID NOS: 58-87 may be adapted for use in the 2+1 mAb-scFv format with or without including K447_on one or both chains. It should be noted that these sequences may further include the M428L/N434S variants. In addition, exemplary CH1-hinge domains, CH1 domains, and hinge domains that can be included in the first or second monomer of the 2+1 mAb-scFv format are provided in SEQ ID NOS: 90-98. Further, useful CL sequences that can be used with this format, i.e., constant domain of the cognate light chains that find use in the subject anti-TL1A×anti-IL23 bsAbs that utilize a Fab binding domain, are provided in SEQ ID NOS: 99 and 100.

7. 2+1 Fab2-scFv-Fc format

One heterodimeric antibody format that finds particular use in subject anti-TL1A×anti-IL23 antibodies provided herein is the 2+1 Fab₂-scFv-Fc format (also referred to as “central-scFv format”) shown in FIG. 1, Panel B. This antibody format includes three antigen-binding domains: two Fab portions and an scFv that is inserted between the VH-CH1 and CH2-CH3 regions of one of the monomers. In some embodiments of this format, the Fab portions each bind IL23 and the “extra” scFv domain binds TL1A. In some embodiments of this format, the Fab portions each bind TL1A and the “extra” scFv domain binds IL23. In some embodiments, the 2+1 Fab2-scFv-Fc format antibody is a trivalent antibody.

In some embodiments of the 2+1 Fab₂-scFv-Fc format, a first monomer includes a standard heavy chain (i.e., VH1-CH1-hinge-CH2-CH3), wherein VH1 is a first variable heavy domain and CH2-CH3 is a first Fc domain. A second monomer includes another first variable heavy domain (VH1), a CH1 domain (and optional hinge), a second Fc domain, and an scFv that includes an scFv variable light domain (VL2), an scFv linker and a scFv variable heavy domain (VH2). The scFv is covalently attached between the C-terminus of the CH1 domain of the second monomer and the N-terminus of the second Fc domain using optional domain linkers (VH1-CH1-[optional linker]-VH2-scFv linker-VH2-[optional linker]-CH2-CH3, or the opposite orientation for the scFv, VH1-CH1-[optional linker]-VL2-scFv linker-VH2-[optional linker]-CH2-CH3). The optional linkers can be any suitable peptide linkers, including, for example, the domain linkers included in Table 6. This embodiment further utilizes a common light chain that includes a variable light domain (VL1) and a constant light domain (CL). In some embodiments, the format includes two identical common light chains (each VL1-CL), wherein each of the common light chains associates with one of the VH1-CH1 of the first and second monomers to form two identical Fabs. In some embodiments, the identical Fabs each bind IL23, and the scFv binds TL1A. In some embodiments, the identical Fabs each bind TL1A, and the scFv binds IL23. As for many of the embodiments herein, these constructs can include skew variants, pI variants, ablation variants, additional Fc variants, etc. as desired and described herein.

In some embodiments, the first and second Fc domains of the 2+1 Fab2-scFv-Fc format antibody are variant Fc domains that include heterodimerization skew variants (e.g., a set of amino acid substitutions as shown in Tables 12-17 and 5). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in Table 1). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in Tables 12-17 and 20). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 Fab₂-scFv-Fc format antibody provided herein includes a charged scFv linker (including those shown in Table 4). In some embodiments, the 2+1 Fab₂-scFv-Fc format antibody provided herein includes FcRn variants M428L/N434S, wherein numbering is according to EU numbering.

In exemplary embodiments 2+1 Fab₂-scFv-Fc format antibody includes a combination of amino acid modifications as depicted in Table 5. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 Fab²-scFv-Fc format antibody provided herein includes a (GKPGS)₄ (SEQ ID NO: 16) charged scFv linker. In some embodiments, the 2+1 Fab₂-scFv-Fc format antibody provided herein includes FcRn variants M428L/N434S, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267 K, and the second Fc domain comprises amino acid variants S364K/E357Q/E233P/L234V/L235A/G236del/S267K, wherein numbering is according to EU numbering.

In some embodiments, the scFv of the second monomer of the 2+1 Fab₂-scFv-Fc format antibody is a TL1A binding domain and the VH1 of the first and second monomer and the VL1 of the common light chain each form binding domains that bind IL23. In some embodiments, the scFv of the second monomer of the 2+1 Fab₂-scFv-Fc format antibody is a IL23 binding domain and the VH1 of the first and second monomer and the VL1 of the common light chain each form binding domains that bind TL1A.

Any suitable TL1A binding domain and IL23 domain can be included in an anti-TL1A×anti-IL23 antibody in the 2+1 Fab₂-scFv-Fc format, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and IL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

### 8. 2+1 Fab2-Fc×scFv-Fc Format

One heterodimeric antibody format that finds particular use in the subject anti-anti-TL1A×anti-IL23 antibodies provided herein is the 2+1 Fab₂-Fc×scFv-Fc format (also referred to as “stacked bottle opener”) shown in FIG. 1, Panel F. This format includes a first monomer, a second monomer and a common light chain. The first monomer includes, from N- to C-terminus: a VH1-CH1-domain linker-VH1-CH1-hinge-CH2-CH3, wherein the VH1s are each a first variable heavy domain and the CH2-CH3 is a first variant Fc domain. The domain linker can be any useful domain linker (see, e.g., Table 6). The second monomer includes a single-chain Fv (“scFv”) covalently attached to a second variant Fc domain by a domain linker (scFv-domain linker-CH2-CH3). The common light chain include a VL1-CL wherein the VL1 is a first variable light domain. The scFv of the second monomer includes a second variable heavy domain (VH2) attached to a second variable light domain (VL2) by an scFv linker. In some embodiments, this format includes two identical common light chains (VL1-CL). In this embodiment, the two VH1-CH1s of the first monomer each interact with a common light chain to form two identical Fabs that are first antigen-binding domains, and the VH2 and VL2 form a second antigen-binding domain. In some embodiments, each of the first antigen-binding domains are IL23 binding domains, and the second antigen-binding domain is a TL1A binding domain. In some embodiments, each of the first antigen-binding domains are TL1A binding domains, and the second antigen-binding domain is an IL23 binding domain. As for many of the embodiments herein, these constructs can include skew variants, pI variants, ablation variants, additional Fc variants, etc. as desired and described herein.

In some embodiments, the first and second Fc domains of the 2+1 Fab2-Fc×scFv-Fc format antibody are variant Fc domains that include heterodimerization skew variants (e.g., a set of amino acid substitutions as shown in Tables 12-17 and 5). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fe domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in Table 1). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in Tables 12-17 and 20). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the 2+1 Fab2-Fc×scFv-Fc format antibody provided herein includes a charged scFv linker (including those shown in Table 4). In some embodiments, the 2+1 Fab2-scFv-Fc format antibody provided herein includes FcRn variants M428L/N434S, wherein numbering is according to EU numbering.

In exemplary embodiments, the 2+1 Fab2-Fc×scFv-Fc format antibody includes a combination of amino acid modifications as depicted in Table 5. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 Fab2-scFv-Fc format antibody provided herein includes a (GKPGS)₄ charged scFv linker (SEQ ID NO: 16). In some embodiments, the 2+1 Fab2-scFv-Fc format antibody provided herein includes FcRn variants M428L/N434S, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/5267 K, and the second Fc domain comprises amino acid variants S364K/E357Q/E233P/L234V/L235A/G236del/S267K, wherein numbering is according to EU numbering.

In some embodiments, the scFv of the second monomer of the 2+1 Fab2-Fc×scFv-Fc format antibody is a TL1A binding domain and the VH1 of the first and second monomer and the VL1 of the common light chain each form binding domains that bind IL23. In some embodiments, the scFv of the second monomer of the 2+1 Fab2-Fc×scFv-Fc format antibody is an IL23 binding domain and the VH1 of the first and second monomer and the VL1 of the common light chain each form binding domains that bind TL1A.

Any suitable TL1A binding domain and IL23 domain can be included in an anti-TL1A×anti-IL23 antibody in the 2+1 Fab2-Fc×scFv-Fc format, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and IL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

Exemplary Fc domain sequences (described in section G.1.) that are useful in 2+1 Fab2-Fc×scFv-Fc format antibodies are provided in SEQ ID NOS: 58-87. In addition, exemplary CH1-hinge domains, CH1 domains, and hinge domains that can be included in the first or second monomer of the 2+1 Fab2-Fc×scFv-Fc format are provided in SEQ ID NOS: 90-98. Further, useful CL sequences that can be used with this format, i.e., constant domain of the cognate light chains that find use in the subject anti-TL1A×anti-IL23 bsAbs that utilize a Fab binding domain, are provided in SEQ ID NOS: 99 and 100.

### 9. 2+1 CLC Format

Another heterodimeric antibody format that finds particular use in subject anti-TL1A×anti-IL23 antibodies provided herein is the “2+1 Common Light Chain” or “2+1 CLC” format, which is depicted in FIG. 1, Panel D. The 2+1 CLC format includes a first monomer that includes a VH1-CH1-linker-VH1-CH1-hinge-CH2-CH3, wherein the VH1s are each a first variable heavy domain and CH2-CH3 is a first Fc domain; a second monomer that includes a VH2-CH1-hinge-CH2-CH3, wherein VH2 is a second variable heavy domain and CH2-CH3 is a second Fc domain; and a third monomer that includes a “common light chain” VL-CL, wherein VL is a common variable light domain and CL is a constant light domain. The VL pairs with each of the VH1s of the first monomer to form two first binding domains, each with a first antigen-binding specificity; and the VL pairs with the VH2 to form a second binding domain with a second antigen-binding specificity. The linker of the first monomer can be any suitable linker, including any one of the domain linkers or combinations thereof described in Table 6. In some embodiments, the 2+1 CLC format antibody is a trivalent antibody.

In some embodiments, the first and second Fc domains of the 2+1 CLC format are variant Fc domains that include heterodimerization skew variants (e.g., a set of amino acid substitutions as shown in Tables 12-17 and 5). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in Table 1). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in Table 20). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 2+1 CLC format antibody provided herein further includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the 2+1 CLC format antibody provided herein further includes FcRn variants M428L/N434S, wherein numbering is according to EU numbering.

In some embodiments, the CH1-hinge-CH2-CH3 of the second monomer comprises amino acid variants L368D/K370S/N208D/Q295E/N384D/Q418E/N421D/E233P/L234V/L235A/G236del/S267 K, and the first Fc domain comprises amino acid variants S364K/E357Q/E233P/L234V/L235A/G236del/S267K, wherein numbering is according to EU numbering.

In some embodiments, each of the two first binding domains binds IL23 and the second binding domain binds TL1A. In some embodiments, each of the two first binding domains binds TL1A and the second binding domain binds IL23. Any suitable TL1A binding domain and IL23 domain can be included in an anti-TL1A×anti-IL23 antibody in the 2+1 CLC format, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and IL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

10. Dual scFv Formats

One heterodimeric antibody format that finds particular use in the subject bispecific anti-TL1A×anti-IL23 antibodies is the dual scFv format, as shown in FIG. 1, Panel G. In this embodiment, the heterodimeric bispecific antibody is made up of two scFv-Fc monomers (both in either (vh-scFv linker-vl-[optional domain linker]-CH2-CH3) format or (vl-scFv linker-vh-[optional domain linker]-CH2-CH3) format, or with one monomer in one orientation and the other in the other orientation. All ABDs are in the scFv format in a dual scFv format antibody.

In addition, the Fc domains of the dual scFv format comprise skew variants (e.g. a set of amino acid substitutions as shown in Tables 12-17 and 5, with particularly useful skew variants being selected from the group consisting of S364K/E357Q:L368D/K370S; L368D/K370S:S364K; L368E/K370S:S364K; T411T/E360E/Q362E:D401K; L368D/K370S:S364K/E357L, K370S:S364K/E357Q, T366S/L368A/Y407V:T366W and T366S/L368A/Y407V/Y349C:T366W/S354C), optionally ablation variants (including those shown in Table 1), optionally charged scFv linkers (including those shown in Table 4) and the heavy chain comprises pI variants (including those shown in Table 20).

In some embodiments, the dual scFv format includes skew variants, pI variants, and ablation variants. Accordingly, some embodiments include formats that comprise: a) a first monomer that comprises the skew variants S364K/E357Q, the ablation variants E233P/L234V/L235A/G236del/S267K, and a scFv that binds a first antigen (VH1-scFv linker-VL1-[optional domain linker]-CH2-CH3 or VL1-scFv linker-VH1-[optional domain linker]-CH2-CH3) and b) a first monomer that comprises the skew variants L368D/K370S, the ablation variants E233P/L234V/L235A/G236del/S267K, and a scFv that binds a second antigen (VH1-scFv linker-VL1-[optional domain linker]-CH2-CH3 or VL1-scFv linker-VH1-[optional domain linker]-CH2-CH3). pI variants can be as outlined herein, but most common will be charged scFv linkers of opposite charge for each monomer. FcRn variants, particularly 428L/434S, can optionally be included.

Any suitable TL1A binding domain and IL23 domain can be included in an anti-TL1A×anti-IL23 antibody in the dual scFv format, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and TL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

### 11. One-Armed Central-scFv

One heterodimeric antibody format that finds particular use in subject anti-TL1A×anti-IL23 antibodies provided herein is the one-armed central-scFv format shown in FIG. 1, Panel K. In this embodiment, one monomer comprises just an Fc domain, while the other monomer includes a Fab domain (a first antigen-binding domain), a scFv domain (a second antigen-binding domain) and an Fc domain, where the scFv domain is inserted between the Fc domain and the Fc domain.

In this embodiment, one monomer comprises a first heavy chain comprising a first variable heavy domain, a CH1 domain and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using domain linkers, in either orientation, VH1-CH1-[optional domain linker]-VH2-scFv linker-VL2-[optional domain linker]-CH2-CH3 or VH1-CH1-[optional domain linker]-VL2-scFv linker-VH2-[optional domain linker]-CH2-CH3. The second monomer comprises an Fc domain (CH2-CH3). This embodiment further utilizes a light chain, comprising a variable light domain and a constant light domain, that associates with the heavy chain to form a Fab. As for many of the embodiments herein, these constructs include skew variants, pI variants, ablation variants, additional Fc variants, etc. as desired and described herein.

Any suitable TL1A binding domain and IL23 domain can be included in an anti-TL1A×anti-IL23 antibody in the one-armed central-scFv format, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and IL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

12. One-Armed scFv-mAb Format

One heterodimeric antibody format that finds particular use in subject anti-TL1A×anti-IL23 antibodies provided herein is the one-armed mAb-scFv format shown in FIG. 1, Panel H. This format includes: 1) a first monomer that comprises an “empty” Fc domain; 2) a second monomer that includes a first variable heavy domain (VH), a scFv domain (a second antigen-binding domain) and an Fc domain, where the scFv domain is attached to the N-terminus of the first variable heavy domain; and 3) a light chain that includes a first variable light domain and a constant light domain. The first variable heavy domain and the first variable light domain form a first antigen-binding domain and the scFv is a second antigen-binding domain. In this format, one of the first antigen-binding domain and second binding domain binds TL1A, and the other antigen-binding domain binds IL23. As for many of the embodiments herein, these constructs include skew variants, pI variants, ablation variants, additional Fc variants, etc. as desired and described herein.

Any suitable TL1A binding domain and IL23 domain can be included in an anti-TL1A×anti-IL23 antibody in the one-armed scFv-mAb format, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and IL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

13. scFv-mAb Format

One heterodimeric antibody format that finds particular use in subject anti-TL1A×anti-IL23 antibodies provided herein is the mAb-scFv format shown in FIG. 1, Panel I. In this embodiment, the format relies on the use of a N-terminal attachment of a scFv to one of the monomers, thus forming a third antigen-binding domain, wherein the Fab portions of the two monomers each bind one target and the “extra” scFv domain binds a different target.

In this embodiment, the first monomer comprises a first heavy chain (comprising a variable heavy domain and a constant domain), with a N-terminally covalently attached scFv comprising a scFv variable light domain, an scFv linker and a scFv variable heavy domain in either orientation ((vh1-scFv linker-vl1-[optional domain linker]-vh2-CH1-hinge-CH2-CH3) or (with the scFv in the opposite orientation) ((vl1-scFv linker-vh1-[optional domain linker]-vh2-CH1-hinge-CH2-CH3)). The second monomer comprises a heavy chain VH2-CH1-hinge-CH2-CH3. This embodiment further utilizes a common light chain, comprising a variable light domain and a constant light domain, that associates with the heavy chains to form two identical Fabs. As for many of the embodiments herein, these constructs include skew variants, pI variants, ablation variants, additional Fc variants, etc. as desired and described herein.

Any suitable TL1A binding domain and IL23 domain can be included in an anti-TL1A×anti-IL23 antibody in the scFv-mAb format, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and IL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

14. mAb-Fv Format

One heterodimeric antibody format that finds particular use in subject anti-TL1A×anti-IL23 antibodies provided herein is the mAb-Fv format (FIG. 1, Panel L). In this embodiment, the format relies on the use of a C-terminal attachment of an “extra” variable heavy domain to one monomer and the C-terminal attachment of an “extra” variable light domain to the other monomer, thus forming a third antigen-binding domain (i.e. an “extra” Fv domain), wherein the Fab portions of the two monomers bind one antigen (TL1A or IL23) and the “extra” Fv domain binds the other antigen (TL1A or IL23).

In this embodiment, the first monomer comprises a first heavy chain, comprising a first variable heavy domain and a first constant heavy domain comprising a first Fc domain, with a first variable light domain covalently attached to the C-terminus of the first Fc domain using a domain linker (vh1-CH1-hinge-CH2-CH3-[optional linker]-vl2). The second monomer comprises a second variable heavy domain, a second constant heavy domain comprising a second Fc domain, and a third variable heavy domain covalently attached to the C-terminus of the second Fc domain using a domain linker (vh1-CH1-hinge-CH2-CH3-[optional linker]-vh2. This embodiment further utilizes a common light chain comprising a variable light domain and a constant light domain, which associates with the heavy chains to form two identical Fabs that include two identical Fvs. The two C-terminally attached variable domains make up the “extra” third Fv. As for many of the embodiments herein, these constructs include skew variants, pI variants, ablation variants, additional Fc variants, etc. as desired and described herein.

Any suitable TL1A binding domain and IL23 domain can be included in an anti-TL1A×anti-IL23 antibody in the mAb-Fv format, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and TL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

### 15. Central-Fv Format

One heterodimeric antibody format that finds particular use in subject anti-TL1A×anti-IL23 antibodies provided herein is the central-Fv format shown in FIG. 1, Panel M. In this embodiment, the format relies on the use of an inserted Fv domain thus forming an “extra” third antigen-binding domain, wherein the Fab portions of the two monomers bind one antigen (TL1A or IL23) and the “extra” central-Fv domain binds the other antigen (TL1A or IL23). The Fv domain is inserted between the Fc domain and the CH1-Fv region of the monomers, thus providing a third antigen-binding domain, wherein each monomer contains a component of the Fv (e.g., one monomer comprises a variable heavy domain and the other a variable light domain of the “extra” central Fv domain).

In this embodiment, one monomer comprises a first heavy chain comprising a first variable heavy domain, a CH1 domain, and Fc domain and an additional variable light domain. The additional variable light domain is covalently attached between the C-terminus of the CH1 domain of the heavy constant domain and the N-terminus of the first Fc domain using domain linkers (vh1-CH1-[optional linker]-vl2-hinge-CH2-CH3). The other monomer comprises a first heavy chain comprising a first variable heavy domain, a CH1 domain and Fc domain and an additional variable heavy domain (vh1-CH1-[optional linker]-vh2-hinge-CH2-CH3). The additional variable heavy domain is covalently attached between the C-terminus of the CH1 domain of the heavy constant domain and the N-terminus of the first Fc domain using domain linkers. This embodiment utilizes a common light chain, comprising a variable light domain and a constant light domain, that associates with the heavy chains to form two identical Fabs that each bind one antigen (TL1A or IL23). The additional variable heavy domain and additional variable light domain form an “extra” central Fv that binds the other antigen (TL1A or IL23). As for many of the embodiments herein, these constructs include skew variants, pI variants, ablation variants, additional Fc variants, etc. as desired and described herein.

Any suitable TL1A binding domain and IL23 domain can be included in an anti-TL1A×anti-IL23 antibody in the central-Fv format, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and IL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

### 16. Non-Heterodimeric Bispecific Antibodies

The anti-TL1A×anti-IL23 antibodies provided herein can also be included in non-heterodimeric bispecific formats (see FIG. 1, Panel J). In this format, the anti-TL1A×anti-IL23 includes: 1) a first monomer comprising a VH1-CH1-hinge-CH2-CH3; 2) a second monomer comprising a VH2-CH1-hinge-CH2-CH3; 3) a first light chain comprising a VL1-CL; and 4) a second light chain comprising a VL2-CL. In such embodiments, the VH1 and VL1 form a first antigen-binding domain and VH2 and VL2 form a second antigen-binding domain. One of the first or second antigen-binding domains binds TL1A and the other antigen-binding domain binds IL23.

Any suitable TL1A binding domain and IL23 domain can be included in an anti-TL1A×anti-IL23 antibody in the non-heterodimeric bispecific antibody format, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and TL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

### 17. Trident Format

In some embodiments, the anti-TL1A×anti-TL23 antibodies provided herein are in the “Trident” format as generally described in WO2015/184203, hereby expressly incorporated by reference in its entirety and in particular for the Figures, Legends, definitions and sequences of “Heterodimer-Promoting Domains” or “HPDs”, including “K-coil” and “E-coil” sequences. Tridents rely on using two different HPDs that associate to form a heterodimeric structure as a component of the structure, see FIG. 1, Panel N. In this embodiment, the Trident format include a “traditional” heavy and light chain (e.g., VH1-CH1-hinge-CH2-CH3 and VL1-CL), a third chain comprising a first “diabody-type binding domain” or “DART®”, VH2-(linker)-VL3-HPD1 and a fourth chain comprising a second DART®, VH3-(linker)-(linker)-VL2-HPD2. The VH1 and VL1 form a first ABD, the VH2 and VL2 form a second ABD, and the VH3 and VL3 form a third ABD. In some cases, as is shown in FIG. 1, Panel P, the second and third ABDs bind the same antigen.

Any suitable TL1A binding domain and IL23 domain can be included in an anti-TL1A×anti-IL23 antibody in the trident format, including any of the TL1A binding domains provided herein or a variant thereof (see, e.g., Tables 44, 7-10, 22, 23, 26, 29-34, and 48, and VH sequences in SEQ ID NOS: 1427-1823 and 2316-2330 and VL sequences in SEQ ID NOS: 1824-1923, and VH/VL pairs set forth in SEQ ID NOS: 2331-2363, or see, e.g., Tables 7-10 and 23) and TL23 binding domains provided herein or a variant thereof (see, e.g., Tables 44, 11, 24-26, 29-34, and 49 and the VH sequences in SEQ ID NOS: 1927-2132 and VL sequences in SEQ ID NOS: 2133-2232, and VH/VL pairs set forth in SEQ ID NOS: 2364-2379, or see, e.g., Table 11, or see, e.g., SEQ ID NOS: 90-95 and FIG. 5).

### V. Nucleic Acids and Methods of Making Antibodies

In another aspect, provided herein are nucleic acid compositions encoding the anti-TL1A antibodies, the anti-IL23 antibodies, or the anti-TL1A×anti-TL23 antibodies provided herein, or nucleic acid compositions encoding any monomer or chain of the anti-TL1A antibodies, the anti-TL23 antibodies, or the anti-TL1A×anti-TL23 antibodies provided herein individually (e.g., a nucleic acid encoding a light chain, a nucleic acid encoding a heavy chain, a nucleic acid encoding a variable light domain, or a nucleic acid encoding a variable heavy domain). A nucleic acid composition may refer to one or multiple polynucleotides.

For heterodimeric antibodies, the nucleic acid compositions will depend on the format and scaffold of the heterodimeric antibody. Thus, for example, when the format requires three amino acid sequences, such as for the 1+1 Fab-scFv-Fc, 2+1 mAb-scFv, 2+1 Fab2-scFv-Fc, and 2+1 Fab2-Fc×scFv-Fc formats, three polynucleotides can be incorporated into one or more expression vectors for expression. In exemplary embodiments, each polynucleotide is incorporated into a different expression vector.

The nucleic acids encoding the components of the binding domains and antibodies disclosed herein can be incorporated into expression vectors, and depending on the host cells used to produce the heterodimeric antibodies of the invention. Generally, the nucleic acids are operably linked to any number of regulatory elements (promoters, origin of replication, selectable markers, ribosomal binding sites, inducers, etc.). The expression vectors can be extra-chromosomal or integrating vectors.

The polynucleotides and/or expression vectors of the invention are then transformed into any number of different types of host cells, including mammalian, bacterial, yeast, insect and/or fungal cells, with mammalian cells (e.g., CHO cells), finding use in many embodiments. Host cells comprising the polynucleotides and/or expression vectors can then be cultured under conditions wherein the antibody is expressed, and the antibody can be recovered (e.g., from the cells or the cell culture medium. For example, the cells can secrete the antibody into cell culture medium, and the antibody can be recovered from the cell culture medium. Such methods can further comprise purifying the antibody.

In some embodiments, polynucleotides encoding each monomer of the antibody (e.g., heterodimeric bispecific antibody) are each contained within a single expression vector, generally under different or the same promoter controls. In embodiments of particular use in the present invention, each of these polynucleotides are contained on different expression vectors. As shown herein and in U.S. 62/025,931, hereby incorporated by reference, different vector ratios can be used to drive heterodimer formation. That is, surprisingly, while the proteins comprise first monomer: second monomer:light chains (in the case of many of the embodiments herein that have three polypeptides comprising the heterodimeric antibody) in a 1:1:2 ratio, these are not the ratios that give the best results.

The antibodies provided herein are made by culturing host cells comprising the expression vector(s). Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “1+1 Fab-scFv-Fc” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

### VI. Biological and Biochemical Functionality of the Anti-TL1A Antibodies, the Anti-IL23 Antibodies, and the Anti-TL1A×Anti-IL23 Antibodies

Generally, the anti-TL1A antibodies described herein can be administered to subjects with a TL1A-associated disease, the anti-IL23 antibodies described herein can be administered to subjects with an IL23-associated disease, and the anti-TL1A×anti-IL23 antibodies described herein or the combinations of the anti-TL1A and anti-IL23 antibodies described herein can be administered to subjects with a TL1A- and/or IL-23 associated disease, and efficacy can be assessed in a number of ways as described herein. For example, the efficacy of the anti-TL1A antibodies, anti-IL23 antibodies, and/or anti-TL1A×anti-IL23 antibodies described herein to block, inhibit, attenuate, or reduce TL1A and/or IL23-mediated signaling can be assessed using a luciferase reporter system, as described elsewhere herein. Likewise, the efficacy of the anti-TL1A antibodies, anti-IL23 antibodies, and/or anti-TL1A×anti-IL23 antibodies described herein to block, inhibit, attenuate, or reduce TL1A and/or IL23 protein levels in circulation and/or diseased tissues can be evaluated by such assays as ELISA and/or immunohistochemistry.

Disclosed herein are antibodies that bind human TL1A with high affinity, antibodies that bind human IL23 (human IL23p19) with high affinity, and antibodies that bind human TL1A and human IL23 (human IL23p19) with high affinity.

In some embodiments, an antibody disclosed herein specifically binds human TL1A with a K_{D} of less than about 2E-10 M, less than about 9E-11 M, less than about 8E-11 M, or less than about 7E-11 M as measured by surface plasmon resonance or Biacore at 25° C. (as described in the examples). In some embodiments, an antibody disclosed herein specifically binds human TL1A with a K_{D} of less than about 6.5E-11 M, less than about 6.4 E-11 M, or less than about 6.3E-11 M as measured by surface plasmon resonance or Biacore at 25° C. In some embodiments, an antibody disclosed herein specifically binds human TL1A with a K_{D} of less than about 4.5E-11 M, less than about 4.4 E-11 M, or less than about 4.3E-11 M as measured by surface plasmon resonance or Biacore at 25° C. In some embodiments, an antibody disclosed herein specifically binds human TL1A with a K_{D} of less than about 3E-11 M, less than about 2.9E-11 M, less than about 2.8E-11 M, or less than about 2.7E-11 M as measured by surface plasmon resonance or Biacore at 25° C. In some embodiments, an antibody disclosed herein specifically binds human TL1A with a K_{D} of less than about 3E-11 M as measured by surface plasmon resonance or Biacore at 25° C. In some embodiments, an antibody disclosed herein specifically binds human TL1A with a K_{D} of less than about 2.9E-11 M as measured by surface plasmon resonance or Biacore at 25° C. In some embodiments, an antibody disclosed herein specifically binds human TL1A with a K_{D} of less than about 2.8E-11 M as measured by surface plasmon resonance or Biacore at 25° C. In some embodiments, an antibody disclosed herein specifically binds human TL1A with a K_{D} of less than about 2.7E-11 M as measured by surface plasmon resonance or Biacore at 25° C. In some embodiments, an antibody disclosed herein specifically binds human TL1A with a K_{D} of between about 2E-11 M and about 7E-11M, between about 2.5E-11 M and about 7E-11 M, or between about 2.5E-11 M and about 6.5E-11 M as measured by surface plasmon resonance or Biacore at 25° C. In some embodiments, an antibody disclosed herein specifically binds human TL1A with a K_{D} of between about 4E-11 M and about 6.5E-11 M as measured by surface plasmon resonance or Biacore at 25° C. In some embodiments, an antibody disclosed herein specifically binds human TL1A with a K_{D} of between about 2.5E-11 M and about 4.5E-11 M as measured by surface plasmon resonance or Biacore at 25° C. In some embodiments, an antibody disclosed herein specifically binds human TL1A with a K_{D} of between about 2E-11 M and about 3E-11 M as measured by surface plasmon resonance or Biacore at 25° C. In some embodiments, an antibody disclosed herein specifically binds human TL1A with a K_{D} of between about 4E-11 M and about 5E-11 M as measured by surface plasmon resonance or Biacore at 25° C. In some embodiments, an antibody disclosed herein specifically binds human TL1A with a K_{D} of between about 6E-11 M and about 7E-11 M as measured by surface plasmon resonance or Biacore at 25° C. In some embodiments, an antibody disclosed herein specifically binds human TL1A with a K_{D} of between about 2.5E-11 M and about 3E-11 M as measured by surface plasmon resonance or Biacore at 25° C. In some embodiments, an antibody disclosed herein specifically binds human TL1A with a K_{D} of between about 2.5E-11 M and about 3.5E-11 M as measured by surface plasmon resonance or Biacore at 25° C. In some embodiments, an antibody disclosed herein specifically binds human TL1A with a K_{D} of between about 2.5E-11 M and about 2.7E-11 M as measured by surface plasmon resonance or Biacore at 25° C.

In some embodiments, an antibody disclosed herein specifically binds human IL23 with a K_{D} of less than about 1.1E-10 M as measured by surface plasmon resonance or Biacore at 25° C. (as described in the examples). In some embodiments, an antibody disclosed herein specifically binds human IL23 with a K_{D} of less than about 7.5E-11 M, less than about 7.4E-11 M, less than about 7E-11 M, less than about 6.5E-11 M, or less than about 6E-11 M as measured by surface plasmon resonance or Biacore at 25° C. In some embodiments, an antibody disclosed herein specifically binds human IL23 with a K_{D} of less than about 6E-11 M, less than about 5.9E-11 M, less than about 5.8E-11 M, or less than about 5.7E-11 M as measured by surface plasmon resonance or Biacore at 25° C. In some embodiments, an antibody disclosed herein specifically binds human IL23 with a K_{D} of less than about 6E-11 M as measured by surface plasmon resonance or Biacore at 25° C. In some embodiments, an antibody disclosed herein specifically binds human IL23 with a K_{D} of less than about 5.9E-11 M as measured by surface plasmon resonance or Biacore at 25° C. In some embodiments, an antibody disclosed herein specifically binds human IL23 with a K_{D} of less than about 5.8E-11 M as measured by surface plasmon resonance or Biacore at 25° C. In some embodiments, an antibody disclosed herein specifically binds human IL23 with a K_{D} of less than about 5.7E-11 M as measured by surface plasmon resonance or Biacore at 25° C. In some embodiments, an antibody disclosed herein specifically binds human IL23 with a K_{D} of less than about 3E-11 M, less than about 2.9E-11 M, less than about 2.8E-11 M, or less than about 2.7E-11 M as measured by surface plasmon resonance or Biacore at 25° C. In some embodiments, an antibody disclosed herein specifically binds human IL23 with a K_{D} of between about 2.4E-11 and about 1E-10 M as measured by surface plasmon resonance or Biacore at 25° C. In some embodiments, an antibody disclosed herein specifically binds human IL23 with a K_{D} of between about 2.4E-11 and about 5.7E-11 M as measured by surface plasmon resonance or Biacore at 25° C. In some embodiments, an antibody disclosed herein specifically binds human IL23 with a K_{D} of between about 2.4E-11 and about 2.7E-11 M as measured by surface plasmon resonance or Biacore at 25° C. In some embodiments, an antibody disclosed herein specifically binds human IL23 with a K_{D} of between about 2E-11 and about 3E-11 M as measured by surface plasmon resonance or Biacore at 25° C. In some embodiments, an antibody disclosed herein specifically binds human IL23 with a K_{D} of between about 5E-11 and about 6E-11 M as measured by surface plasmon resonance or Biacore at 25° C. In some embodiments, an antibody disclosed herein specifically binds human IL23 with a K_{D} of between about 5.5E-11 and about 6E-11 M as measured by surface plasmon resonance or Biacore at 25° C. In some embodiments, an antibody disclosed herein specifically binds human IL23 with a K_{D} of between about 5.5E-11 and about 5.8E-11 M as measured by surface plasmon resonance or Biacore at 25° C. In some embodiments, an antibody disclosed herein specifically binds human IL23 with a K_{D} of between about 5.6E-11 and about 5.7E-11 M as measured by surface plasmon resonance or Biacore at 25° C.

Disclosed herein are antibodies that inhibit TL1A (e.g., human TL1A) with strong potency, antibodies that inhibit IL23 (e.g., human IL23, e.g., human IL23p19) with high potency, and antibodies that bind inhibit TL1A (e.g., human TL1A) and IL23 (e.g., human IL23, e.g., human IL23p19) with high potency. Inhibitory activity of an individual Fab binding domain within a bispecific modality would be expected to be ~50% less than the bivalent antibody variant in standard lgG format since binding reflects monovalent versus bivalent neutralization ability. Surprisingly, some antibodies disclosed herein in monovalent format (e.g., as part of a bispecific anti-TL1A×anti-1123 antibody) showed efficacy in inhibiting TL1A activity comparable to or better than comparator bivalent monoclonal antibodies. Likewise, surprisingly, some antibodies disclosed herein in monovalent format (e.g., as part of a bispecific anti-TL1A×anti-IL23 antibody) showed efficacy in inhibiting IL23 activity comparable to or better than comparator bivalent monoclonal antibodies.

In some embodiments, an antibody disclosed herein inhibits TL1A activity in a TL1A luciferase reporter assay (like described in the examples) with an IC₅₀ of less than about 4.5 nM. In some embodiments, an antibody disclosed herein inhibits TL1A activity in a TL1A luciferase reporter assay with an IC₅₀ of less than about 4.3 nM, less than about 4.2 nM, or less than about 4.1 nM. In some embodiments, an antibody disclosed herein inhibits TL1A activity in a TL1A luciferase reporter assay with an IC₅₀ of less than about 4 nM, less than about 3.9 nM, less than about 3.8 nM, or less than about 3.7 nM. In some embodiments, an antibody disclosed herein inhibits TL1A activity in a TL1A luciferase reporter assay with an IC₅₀ of less than about 3 nM, less than about 2.9 nM, or less than about 2.8 nM. In some embodiments, an antibody disclosed herein inhibits TL1A activity in a TL1A luciferase reporter assay with an IC₅₀ of between about 2.5 nM and about 4.5 nM. In some embodiments, an antibody disclosed herein inhibits TL1A activity in a TL1A luciferase reporter assay with an IC₅₀ of between about 3 nM and about 5 nM. In some embodiments, an antibody disclosed herein inhibits TL1A activity in a TL1A luciferase reporter assay with an IC₅₀ of between about 2.5 nM and about 4 nM. In some embodiments, an antibody disclosed herein inhibits TL1A activity in a TL1A luciferase reporter assay with an IC₅₀ of between about 3.5 nM and about 4.5 nM. In some embodiments, an antibody disclosed herein inhibits TL1A activity in a TL1A luciferase reporter assay with an IC₅₀ of between about 3.5 nM and about 5.5 nM. In some embodiments, an antibody disclosed herein inhibits TL1A activity in a TL1A luciferase reporter assay with an IC₅₀ of between about 4 nM and about 5 nM.

In some embodiments, an antibody disclosed herein inhibits IL23 activity in an IL23 luciferase reporter assay (like that described in the examples) with an IC₅₀ of less than about 65 pM, less than about 64 pM, or less than about 63 pM. In some embodiments, an antibody disclosed herein inhibits IL23 activity in an IL23 luciferase reporter assay with an IC₅₀ of less than about 60 pM, less than about 59 pM, less than about 58 pM, less than about 57 pM, or less than about 56 pM. In some embodiments, an antibody disclosed herein inhibits IL23 activity in an IL23 luciferase reporter assay with an IC₅₀ of less than about 60 pM. In some embodiments, an antibody disclosed herein inhibits IL23 activity in an IL23 luciferase reporter assay with an IC₅₀ of less than about 59 pM. In some embodiments, an antibody disclosed herein inhibits IL23 activity in an IL23 luciferase reporter assay with an IC₅₀ of less than about 58 pM. In some embodiments, an antibody disclosed herein inhibits IL23 activity in an IL23 luciferase reporter assay with an IC₅₀ of less than about 57 pM. In some embodiments, an antibody disclosed herein inhibits IL23 activity in an IL23 luciferase reporter assay with an IC₅₀ of less than about 56 pM. In some embodiments, an antibody disclosed herein inhibits IL23 activity in an IL23 luciferase reporter assay with an IC₅₀ of less than about 60 pM, less than about 50 pM, less than about 49 pM, or less than about 48 pM. In some embodiments, an antibody disclosed herein inhibits IL23 activity in an IL23 luciferase reporter assay with an IC₅₀ of between about 47 pM and about 63 pM. In some embodiments, an antibody disclosed herein inhibits IL23 activity in an IL23 luciferase reporter assay with an IC₅₀ of between about 47 pM and about 59 pM. In some embodiments, an antibody disclosed herein inhibits IL23 activity in an IL23 luciferase reporter assay with an IC₅₀ of between about 47 pM and about 56 pM. In some embodiments, an antibody disclosed herein inhibits IL23 activity in an IL23 luciferase reporter assay with an IC₅₀ of between about 47 pM and about 50 pM. In some embodiments, an antibody disclosed herein inhibits IL23 activity in an IL23 luciferase reporter assay with an IC₅₀ of between about 45 pM and about 50 pM. In some embodiments, an antibody disclosed herein inhibits IL23 activity in an IL23 luciferase reporter assay with an IC₅₀ of between about 50 pM and about 60 pM. In some embodiments, an antibody disclosed herein inhibits IL23 activity in an IL23 luciferase reporter assay with an IC₅₀ of between about 53 pM and about 57 pM. In some embodiments, an antibody disclosed herein inhibits IL23 activity in an IL23 luciferase reporter assay with an IC₅₀ of between about 54 pM and about 57 pM. In some embodiments, an antibody disclosed herein inhibits IL23 activity in an IL23 luciferase reporter assay with an IC₅₀ of between about 55 pM and about 57 pM. In some embodiments, an antibody disclosed herein inhibits IL23 activity in an IL23 luciferase reporter assay with an IC₅₀ of between about 55 pM and about 56 pM. In some embodiments, an antibody disclosed herein inhibits IL23 activity in an IL23 luciferase reporter assay with an IC₅₀ of between about 50 pM and about 60 pM.

### A. Antibody Compositions for In Vivo Administration

Embodiments of the invention relate to a pharmaceutical composition comprising a pharmaceutically acceptable carrier and any one of the anti-TL1A antibodies described herein, any one of the anti-IL23 antibodies described herein, any one of the anti-TL1A×anti-IL23 antibodies described herein, a combination of any one of the anti-TL1A antibodies described herein and an anti-IL23 antibody, or a combination of any one of the IL23 antibodies described herein and an anti-TL1A antibody. Formulations of the anti-TL1A antibodies, the anti-IL23 antibodies, the anti-TL1A×anti-IL23 antibodies, or the combinations described herein can be prepared for storage by mixing an antibody having the desired degree of purity with optional pharmaceutically acceptable carriers, excipients, or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

### B. Kits and Delivery Devices

In another aspect, provided are kits or vessels or delivery devices comprising any one of the anti-TL1A antibodies described herein, any one of the anti-IL23 antibodies described herein, any one of the anti-TL1A×anti-IL23 antibodies described herein, a combination of any one of the anti-TL1A antibodies described herein and an anti-IL23 antibody, or a combination of any one of the IL23 antibodies described herein and an anti-TL1A antibody or any of the pharmaceutical compositions described herein. Such kits can further comprise, for example: (i) a device for administering the aforementioned antibodies or pharmaceutical compositions; and/or (ii) instructions for use. Containers used in such kits may typically comprise at least one vial, test tube, flask, bottle, syringe, or other suitable container into which one or more of the antibodies or pharmaceutical compositions may be placed, and preferably aliquoted appropriately.

Where a second therapeutic agent is also provided (e.g., combinations of an anti-TL1A antibody and a separate anti-IL23 antibody), the kit may also contain a second, different container in which the second antibody may be placed. Alternatively, multiple compounds can be prepared as a single pharmaceutical composition and packaged in a single container device such as a vial, flask, syringe, bottle, or other suitable single container.

Also provided are drug delivery devices (e.g., plastic or vial, such as a hollow pin or syringe barrel) that can be used to administer the antibody or pharmaceutical composition. The device can introduce a substance into the patient via parenteral routes (such as subcutaneously or intravenously). For example, the injection device may be a syringe (e.g., a prefilled syringe with an antibody or pharmaceutical composition described herein, such as an autoinjector), which may include a syringe and a needle (can be used to pierce skin and/or blood vessels) containing the fluid to be injected (e.g., the antibody or pharmaceutical composition described herein).

### VII. Treatments

Once made, the anti-TL1A×anti-IL23 antibody compositions of the invention find use in a number of applications, for example, in the treatment of a TL1A-associated and/or IL23-associated disease or condition or in the preparation of medicaments for treating such diseases or conditions. Likewise, the anti-TL1A antibody compositions can find use in the treatment of a TL1A-associated disease or condition, and the anti-IL23 antibody compositions can find use in the treatment of an IL23-associated disease or condition, or in the preparation of medicaments for treating such diseases or conditions. Likewise, combinations of an anti-TL1A antibody and an anti-IL23 antibody can find use in the treatment of a TL1A-associated and/or IL23-associated disease or condition or in the preparation of medicaments for treating such diseases or conditions. Examples of a TL1A-associated and/or IL23-associated disease or condition include diseases or conditions associated with a TL1A- and/or IL23-mediated inflammatory response, TL1A- and/or IL23-mediated autoimmune diseases or conditions, and TL1A- and/or IL23-mediated fibrotic diseases or conditions. Some non-limiting examples of a TL1A-associated and/or IL23-associated disease or condition are inflammatory bowel disease, ulcerative colitis, Crohn's disease, fibrostenotic Crohn's disease, systemic sclerosis, interstitial lung disease, primary biliary cholangitis, primary sclerosing cholangitis, rheumatoid arthritis, and atopic dermatitis. Some specific examples of a TL1A-associated and/or IL23-associated disease or condition that can be treated by the compositions disclosed herein are inflammatory bowel diseases such as ulcerative colitis and Crohn's disease.

Once made, the anti-TL1A×anti-IL23 antibody compositions of the invention find use in a number of applications, for example, in the treatment of diseases or conditions associated with a TL1A- and/or IL23-mediated inflammatory response or inflammatory diseases or conditions or in the preparation of medicaments for treating such diseases or conditions. Likewise, the anti-TL1A antibody compositions can find use in the treatment of diseases or conditions associated with a TL1A-mediated inflammatory response or inflammatory diseases or conditions, and the anti-IL23 antibody compositions can find use in the treatment of diseases or conditions associated with an IL23-mediated inflammatory response or inflammatory diseases or conditions, or in the preparation of medicaments for treating such diseases or conditions. Likewise, combinations of an anti-TL1A antibody and an anti-IL23 antibody can find use in the treatment of diseases or conditions associated with a TL1A- and/or IL23-mediated inflammatory response or inflammatory diseases or conditions or in the preparation of medicaments for treating such diseases or conditions. Non-limiting examples of inflammatory diseases or conditions include allergies, ankylosing spondylitis, asthma, atopic dermatitis, autoimmune diseases or conditions, cancer, celiac disease, chronic obstructive pulmonary disease (COPD), chronic peptic ulcer, cystic fibrosis, diabetes mellitus (e.g., type 1 diabetes and type 2 diabetes), glomerulonephritis, gout, hepatitis (e.g., active hepatitis), inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), reperfusion injury, transplantation rejection, psoriasis, pulmonary fibrosis, rheumatic diseases, scleroderma, sinusitis, and tuberculosis.

The anti-TL1A×anti-IL23 antibody compositions of the invention can also find use in the treatment of TL1A- and/or IL23-mediated autoimmune diseases or conditions or in the preparation of medicaments for treating such diseases or conditions. Likewise, the anti-TL1A antibody compositions can find use in the treatment of TL1A-mediated autoimmune diseases or conditions, and the anti-IL23 antibody compositions can find use in the treatment of IL23-mediated autoimmune diseases or conditions, or in the preparation of medicaments for treating such diseases or conditions. Likewise, combinations of an anti-TL1A antibody and an anti-IL23 antibody can find use in the treatment of TL1A- and/or IL23-mediated autoimmune diseases or conditions or in the preparation of medicaments for treating such diseases or conditions. Non-limiting examples of autoimmune diseases or conditions include achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AM AN), Balo's disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatosis (EGPA), cicatricial pemphigoid, Cogan's syndrome, cold agglutinin disease, congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (acne in versa), hypogammaglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, chronic Lyme disease, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry-Romberg syndrome, pars planitis (peripheral uveitis), Parsonnage-Turner syndrome, pemphigus, peripheral neuropathy, perivenous encephalomyelitis, pernicious anemia (PA), POEMS syndrome, polyarteritis nodosa, polyglandular syndrome type I, polyglandular syndrome type II, polyglandular syndrome type III, polymyalgia rheumatica, polymyositis, post-myocardial infarction syndrome, post-pericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome (SPS), subacute bacterial endocarditis (SBE), Susac's syndrome, sympathetic ophthalmia (SO), Takayasu's arteritis, temporal arteritis/giant cell arteritis, thrombocytopenic purpura (TTP), Tolosa-Hunt syndrome (THS), transverse myelitis, type 1 diabetes, ulcerative colitis (UC), undifferentiated connective tissue disease (UCTD), uveitis, vasculitis, vitiligo, and Vogt-Koyanagi-Harada Disease.

The anti-TL1A×anti-IL23 antibody compositions of the invention can also find use in the treatment of TL1A- and/or IL23-mediated fibrotic diseases or conditions or in the preparation of medicaments for treating such diseases or conditions. Likewise, the anti-TL1A antibody compositions can find use in the treatment of TL1A-mediated fibrotic diseases or conditions, and the anti-IL23 antibody compositions can find use in the treatment of IL23-mediated fibrotic diseases or conditions, or in the preparation of medicaments for treating such diseases or conditions. Likewise, combinations of an anti-TL1A antibody and an anti-IL23 antibody can find use in the treatment of TL1A- and/or IL23-mediated fibrotic diseases or conditions or in the preparation of medicaments for treating such diseases or conditions. “Fibrotic disease or condition” means a disease or condition characterized by fibrosis, including but not limited to: respiratory conditions such as pulmonary fibrosis, cystic fibrosis, idiopathic pulmonary fibrosis, progressive massive fibrosis, scleroderma, obliterative bronchiolitis, Hermansky-Pudlak syndrome, asbestosis, silicosis, chronic pulmonary hypertension, AIDS-associated pulmonary hypertension, sarcoidosis, tumor stroma in lung disease, and asthma; chronic liver disease, primary biliary cirrhosis (PBC), schistosomal liver disease, liver cirrhosis; cardiovascular conditions such as hypertrophic cardiomyopathy, dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertensive heart disease, arrhythmogenic right ventricular cardiomyopathy (ARVC), tubulointerstitial and glomerular fibrosis, atherosclerosis, varicose veins, cerebral infarcts; neurological conditions such as gliosis and Alzheimer's disease; muscular dystrophies such as Duchenne muscular dystrophy (DMD) or Beckers muscular dystrophy (BMD); gastrointestinal conditions such as Crohn's disease, microscopic colitis and primary sclerosing cholangitis (PSC); skin conditions such as scleroderma, nephrogenic systemic fibrosis and cutis keloid; arthrofibrosis; Dupuytren's contracture; mediastinal fibrosis; retroperitoneal fibrosis; myelofibrosis; Peyronie's disease; adhesive capsulitis; kidney disease (e.g., renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus); progressive systemic sclerosis (PSS); chronic graft versus host disease; diseases of the eye such as Grave's opthalmopathy, epiretinal fibrosis, retinal fibrosis, subretinal fibrosis (e.g. associated with macular degeneration (e.g. wet age-related macular degeneration (AMD)), diabetic retinopathy, glaucoma, corneal fibrosis, post-surgical fibrosis (e.g., of the posterior capsule following cataract surgery, or of the bleb following trabeculectomy for glaucoma), conjunctival fibrosis, subconjunctival fibrosis; arthritis; fibrotic pre-neoplastic and fibrotic neoplastic disease; and fibrosis induced by chemical or environmental insult (e.g., cancer chemotherapy, pesticides, radiation/cancer radiotherapy).

In one aspect, provided herein are methods of inhibiting a TL1A-mediated inflammatory, autoimmune, and/or fibrotic response or activity using the subject anti-TL1A antibody or anti-TL1A×anti-IL23 antibody or the use of such antibodies in such methods or the use of such antibodies in the preparation of reagents or medicaments for use in such methods. TL1A is a cytokine that is secreted by antigen-presenting cells, T cells, and endothelial cells. TL1A signals through death receptor 3 (DR3), a TNF-family receptor that is found primarily on T cells, natural killer (NK) and NK-T cells, innate lymphoid cells (ILC), fibroblasts, and epithelial cells and potently drives Th1, Th2, Th9 and Th17 responses. In addition, it is induced in antigen-presenting cells by toll like receptor (TLR) ligands and FcR cross-linking and in T cells by T cell receptor (TCR) stimulation. TL1A binding to DR3 on innate immune and T cells leads to an early cytokine response (release of IL-23, IL-1β, IL-17, IL-22, TNF-α, IFN-γ, and IL-13) that sets the stage for inflammation and stimulates innate and adaptive immune response. For instance, through binding to DR3, TL1A potentially drives inflammatory Th1 and Th17 responses. Further, binding of TL1A to DR3 on fibroblasts directly activates fibroblasts and leads to collagen disposition and fibrosis independent of inflammation. In some embodiments the TL1A-mediated response or activity is associated with TL1A/DR3 signaling. In some embodiments, the subject anti-TL1A antibody or anti-TL1A×anti-IL23 antibody blocks, inhibits, attenuates, or reduces TL1A binding to the natural TL1A receptor, DR3. In some embodiments, the subject anti-TL1A antibody or anti-TL1A×anti-IL23 antibody blocks, inhibits, attenuates, or reduces TL1A-mediated signaling. In some embodiments, the subject anti-TL1A antibody or anti-TL1A×anti-IL23 antibody blocks, inhibits, attenuates, or reduces TL1A protein levels in circulation and/or diseased tissues. In some embodiments, the subject anti-TL1A antibody or anti-TL1A×anti-IL23 antibody inhibits TL1A/DR3-mediated apoptosis. In some embodiments, the anti-TL1A antibody or anti-TL1A×anti-IL23 antibody inhibits TL1A/DR3 signaling-mediated inflammation.

In some embodiments, the anti-TL1A antibody or anti-TL1A×anti-IL23 antibody inhibits NFκB signaling. In some embodiments, the anti-TL1A antibody or anti-TL1A×anti-IL23 antibody inhibits TL1A/DR3 signaling-mediated activity, wherein the DR3 is expressed on a lymphocyte, NK cell, NK-T cell, ILC, or epithelial cell. In some embodiments, the anti-TL1A antibody or anti-TL1A×anti-IL23 antibody inhibits TL1A-induced cytokine secretion, optionally wherein the cytokine is selected from interferon-gamma (IFNγ), interleukin-1beta (IL1β), tumor necrosis factor-alpha (TNFα), transforming growth factor-beta (TGFβ), and granulocyte-macrophage colony stimulating factor (GM-CSF). In some embodiments, the anti-TL1A antibody or anti-TL1A×anti-IL23 antibody inhibits the activity of a Th1, Th17, Th2, or Th 9 cell. In some embodiments, the anti-TL1A antibody or anti-TL1A×anti-IL23 antibody inhibits the secretion or production of one or more of the following cytokines: IL10, IL4, IL9, IL13, IL12, IL17, IL18, IL22, IL23, IFNγ, and TNFα. In some embodiments, the anti-TL1A antibody or anti-TL1A×anti-IL23 antibody inhibits TL1A/DR3 signaling-mediated activity in a human subject.

In another aspect, provided herein are methods of inhibiting an IL23-mediated inflammatory, autoimmune, and/or fibrotic response or activity using the subject anti-IL23 antibody or anti-TL1A×anti-IL23 antibody or the use of such antibodies in such methods or the use of such antibodies in the preparation of reagents or medicaments for use in such methods. IL23 is a heterodimeric cytokine composed of a unique p19 subunit and a common p40 subunit shared with IL12. IL23 engages with the heterodimeric IL23 receptor (IL23R; consisting of an IL23R chain and an IL12Rβ1 chain), activates intracellular JAKs (mainly through TYK2 and JAK2) and signal transducer and activator of transcription (STAT) pathways, among other signaling factors, which in turn regulates transcription of downstream genes. IL23 is one of the key promotors of the Th17 cell pathway, which has been implicated in many inflammatory diseases and conditions. In some embodiments, the IL23-mediated response or activity is associated with IL23/IL23R signaling. In some embodiments, the subject anti-IL23 antibody or anti-TL1A×anti-IL23 antibody blocks, inhibits, attenuates, or reduces IL23 binding to the natural IL23 receptor, IL23R. In some embodiments, the subject anti-IL23 antibody or anti-TL1A×anti-IL23 antibody blocks, inhibits, attenuates, or reduces IL23-mediated signaling. In some embodiments, the subject anti-IL23 antibody or anti-TL1A×anti-IL23 antibody blocks, inhibits, attenuates, or reduces IL23 protein levels in circulation and/or diseased tissues. In some embodiments, the subject anti-IL23 antibody or anti-TL1A×anti-IL23 antibody binds to IL23 but does not bind to IL12.

In another aspect, provided herein are methods of inhibiting an inflammatory, autoimmune, and/or fibrotic response or activity mediated by both TL1A and IL23 using the combination of the subject anti-TL1A antibody and anti-IL23 antibody or the subject anti-TL1A×anti-IL23 antibody or the use of such antibodies in such methods or the use of such antibodies in the preparation of reagents or medicaments for use in such methods. Certain inflammatory immune cells express both TL1A and IL23 pathway receptors, and inhibition of both pathways may be needed in order to completely neutralize the proinflammatory function of such inflammatory immune cells. Additionally, inflammatory immune cells that express receptors for either pathway alone can also contribute to inflammation and disease independently. Accordingly, inhibition of both pathways may be beneficial in that it could inhibit larger populations of inflammatory immune cells and/or achieve synergistic inhibition over inhibiting either pathway independently. In some embodiments, the TL1A and IL23-mediated response or activity is associated with TL1A/DR3 and IL23/IL23R signaling. In some embodiments, the combination of the subject anti-TL1A antibody and anti-IL23 antibody or the subject anti-TL1A×anti-IL23 antibody blocks, inhibits, attenuates, or reduces both TL1A binding to DR3 and IL23 binding to IL23R. In some embodiments, the combination of the subject anti-TL1A antibody and anti-IL23 antibody or the subject anti-TL1A×anti-IL23 antibody blocks, inhibits, attenuates, or reduces TL1A- and IL23-mediated signaling. In some embodiments, the combination of the subject anti-TL1A antibody and anti-IL23 antibody or the subject anti-TL1A×anti-IL23 antibody blocks, inhibits, attenuates, or reduces TL1A and IL23 protein levels in circulation and/or diseased tissues.

### A. Administrative Modalities

The antibodies provided herein administered to a subject, in accord with known methods, such as intravenous administration as a bolus or by continuous infusion over a period of time.

Antibodies can be administered via a peripheral route. Routes for administration of antibodies can be, for example, intravenous or subcutaneous.

### B. Treatment Modalities

In the methods of the invention, therapy is used to provide a positive therapeutic response with respect to a disease or condition.

By “positive therapeutic response” is intended an improvement in the disease or condition, and/or an improvement in the signs or symptoms associated with the disease or condition. The positive therapeutic response may be prophylactic in terms of completely or partially preventing a disease or symptom thereof and/or may be therapeutic in terms of effecting a partial or complete cure for a disease and/or symptom thereof. “Treatment,” as used herein, may include treatment of a disease or disorder (e.g. an inflammatory disease, an autoimmune disease, or a fibrotic disease) in a mammal, particularly in a human, and includes: (a) preventing the disease or a symptom of a disease from occurring in a subject which may be predisposed to the disease but has not yet been diagnosed as having it (including diseases that may be associated with or caused by a primary disease); (b) inhibiting the disease, i.e., arresting its development; and (c) relieving the disease, i.e., causing regression of the disease. Treating may refer to any indicia of success in the treatment or amelioration or prevention of the disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. For example, a positive therapeutic response in a subject with moderate to severely active ulcerative colitis could refer to one or more of the following improvements: (1) endoscopic improvement (defined by endoscopy subscore of ≤1 with no friability in the 3-component Modified Mayo Score); (2) a 3-component Modified Mayo Score clinical response in the subject (defined by reduction from baseline ≥2 points and reduction ≥30% in 3-component Modified Mayo Score, accompanied by a reduction ≥1 in rectal bleeding subscore or absolute rectal bleeding subscore ≤1); (3) a 3-component Modified Mayo Score clinical remission in the subject (defined by an endoscopic subscore of 0 or 1, rectal bleeding subscore of 0, and stool frequency subscore of 0 or 1 and not greater than baseline); and (4) histologic remission (defined by Geboes score ≤3.1).

Positive therapeutic responses in any given disease or condition can be determined by standardized response criteria specific to that disease or condition. As described above, response to inflammatory conditions like ulcerative colitis can be assessed using standardized scoring methods that assess symptoms with standard screening methods such as questionnaires, endoscopic procedures, and histologic analysis. Positive therapeutic responses can be determined in an individual subject by comparing sign(s) and/or symptom(s) of a condition after treatment to baseline value(s) before treatment. The term “symptom” refers to a subjective evidence of a disease, such as altered gait, as perceived by the subject. A “sign” refers to objective evidence of a disease as observed by a physician. Positive therapeutic responses can also be determined by comparing a treated population with a control population not receiving an agent under test.

In addition to these positive therapeutic responses, the subject undergoing therapy may experience the beneficial effect of an improvement in the symptoms associated with the disease.

Treatment according to the present invention includes a “therapeutically effective amount” or “therapeutically effective regimen” of the medicaments used. A “therapeutically effective amount” refers to an amount effective, at dosages and for periods of time necessary, to achieve a desired therapeutic result. A “therapeutically effective regimen” refers to a combination of dose, frequency of administration, and route of administration effective to achieve a desired therapeutic result. For example, the desired therapeutic result can be an amount/regimen sufficient to alleviate one or more signs and/or symptoms of a disease in the treated subject, whether by inducing the regression or elimination of such signs and/or symptoms or by inhibiting the progression of such signs and/or symptoms. In some instances, therapeutic efficacy can be observed in an individual patient relative to historical controls or past experience in the same patient. In other instances, therapeutic efficacy can be demonstrated in a preclinical or clinical trial in a population of treated patients relative to a control population of untreated patients.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects. The dose amount may vary depending upon the age and the size of a subject to be administered, target disease, conditions, route of administration, and the like. In certain embodiments, the initial dose may be followed by administration of a second or a plurality of subsequent doses.

Alternatively, this property of a composition may be evaluated by examining the ability of the compound to inhibit cell growth or to induce apoptosis by in vitro assays. A therapeutically effective amount of a therapeutic compound may decrease tumor size, or otherwise ameliorate symptoms in a subject. Such amounts may vary based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the bispecific antibodies used in the present invention depend on the disease or condition to be treated.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 21

| SEQ ID | | |
|---|---|---|
| NO | Type | Description |
| 1 | Protein | Human IL23A |
| 2 | Protein | Mouse IL23A |
| 3 | Protein | Cynomolgus IL23A |
| 4 | Protein | Human TL1A |
| 5 | Protein | Mouse TL1A |
| 6 | Protein | Rat TL1A |
| 7-17 | Protein | Positively charged scFv linkers |
| 18-25 | Protein | Negatively charged scFv linkers |
| 26-29 | Protein | Additional scFv linkers |
| 30 | Skipped | Skipped |
| 31-57 | Protein | Useful domain linkers |
| 58 | Protein | Heterodimeric Fc Backbone 1 monomer 1 (−) |
| 59 | Protein | Heterodimeric Fc Backbone 1 monomer 2 (+) |
| 60 | Protein | Heterodimeric Fc Backbone 2 monomer 1 (−) |
| 61 | Protein | Heterodimeric Fc Backbone 2 monomer 2 (+) |
| 62 | Protein | Heterodimeric Fc Backbone 3 monomer 1 (−) |
| 63 | Protein | Heterodimeric Fc Backbone 3 monomer 2 (+) |
| 64 | Protein | Heterodimeric Fc Backbone 4 monomer 1 (−) |
| 65 | Protein | Heterodimeric Fc Backbone 4 monomer 2 (+) |
| 66 | Protein | Heterodimeric Fc Backbone 5 monomer 1 (−) |
| 67 | Protein | Heterodimeric Fc Backbone 5 monomer 2 (+) |
| 68 | Protein | Heterodimeric Fc Backbone 6 monomer 1 (−) |
| 69 | Protein | Heterodimeric Fc Backbone 6 monomer 2 (+) |
| 70 | Protein | Heterodimeric Fc Backbone 7 monomer 1 (−) |
| 71 | Protein | Heterodimeric Fc Backbone 7 monomer 2 (+) |
| 72 | Protein | Heterodimeric Fc Backbone 8 monomer 1 (−) |
| 73 | Protein | Heterodimeric Fc Backbone 8 monomer 2 (+) |
| 74 | Protein | Heterodimeric Fc Backbone 9 monomer 1 (−) |
| 75 | Protein | Heterodimeric Fc Backbone 9 monomer 2 (+) |
| 76 | Protein | Heterodimeric Fc Backbone 10 monomer 1 (−) |
| 77 | Protein | Heterodimeric Fc Backbone 10 monomer 2 (+) |
| 78 | Protein | Heterodimeric Fc Backbone 11 monomer 1 (−) |
| 79 | Protein | Heterodimeric Fc Backbone 11 monomer 2 (+) |
| 80 | Protein | Heterodimeric Fc Backbone 12 monomer 1 (−) |
| 81 | Protein | Heterodimeric Fc Backbone 12 monomer 2 (+) |
| 82 | Protein | Heterodimeric Fc Backbone 13 monomer 1 |
| 83 | Protein | Heterodimeric Fc Backbone 13 monomer 2 |
| 84 | Protein | Heterodimeric Fc Backbone 14 monomer 1 |
| 85 | Protein | Heterodimeric Fc Backbone 14 monomer 2 |
| 86 | Protein | Heterodimeric Fc Backbone 15 monomer 1 |
| 87 | Protein | Heterodimeric Fc Backbone 15 monomer 2 |
| 88 | Protein | 2 + 1 mAb-scFv Heterodimeric Fc Backbone monomer 1 |
| 89 | Protein | 2 + 1 mAb-scFv Heterodimeric Fc Backbone monomer 2 |
| 90 | Protein | IgG1 CH1(+) |
| 91 | Protein | IgG1 CH1(−) |
| 92 | Protein | IgG2 CH1(+) |
| 93 | Protein | IgG2 CH1(−) |
| 94 | Protein | IgG4 CH1(+) |
| 95 | Protein | IgG4 CH1(−) |
| 96 | Protein | IgG1 hinge |
| 97 | Protein | IgG2 hinge |
| 98 | Protein | IgG4 hinge |
| 99 | Protein | Light chain constant domain - kappa |
| 100 | Protein | Light chain constant domain - lambda |
| 101-176 | Protein | Variable heavy chain, variable light chain, and associated CDR sequences for |
| | | TL1A binding domains |
| 177 | Protein | XENP49698_IL23-A[IL23]_H1L1_Fab-IgG1_PVA_/S267K/M428L/N434S |
| | | Chain 1- IL23-A[IL23]_H1_Fab-IgG1_PVA_/S267K/M428L/N434S |
| 178 | Protein | XENP49698_IL23-A[IL23]_H1L1_Fab-IgG1_PVA_/S267K/M428L/N434S |
| | | Chain 2- IL23-A[IL23]_L1_Light Chain |
| 179-185 | Protein | Variable heavy chain, variable light chain, and associated CDR sequences for |
| | | IL23-A binding domains |
| 186-1019 | Protein | Illustrative sequences for TL1A × IL23 bsAbs in the 1 + 1-Fab-scFv-Fc format |
| 1020-1155 | Protein | Illustrative sequences for TL1A × IL23 bsAbs in the 1 + 1 OrthoFab format |
| 1156-1161 | Protein | Illustrative sequences for TL1A × IL23 bsAbs in the CLC 1 + 1 format |
| 1162-1175 | Protein | Illustrative sequences for TL1A × IL23 bsAbs in additional bispecific formats |
| 1176-1199 | Protein | Illustrative sequences for TL1A × IL23 bsAbs in the CrossMab format |
| 1200-1247 | Protein | Variable heavy chain, variable light chain, and associated CDR sequences for |
| | | additional TL1A binding domains |
| 1248-1290 | Protein | Variable heavy chain, variable light chain, and associated CDR sequences for |
| | | additional IL23 binding domains |
| 1291-1394 | Protein | Illustrative sequences for TL1A × IL23 bsAbs in CrossMab-VH-VL charge-swap |
| | | 1 + 1 format |
| 1395-1398 | Skipped | Skipped |
| 1399-1404 | Protein | Bispecific sequences utilizing TL1A and RSV or IL23 and RSV |
| 1405-1420 | Protein | Sequences for bivalent TL1A mAbs utilized as controls |
| 1421-1424 | Protein | Sequences for bivalent IL23 mAbs utilized as controls |
| 1425-1426 | Protein | Sequences for bivalent IL12 mAbs utilized as controls |
| 1427-1823 | Protein | Sequences for superhumanized and affinity-engineered variable heavy domains |
| | | (H1.1-H1.486) from anti-TL1A clone 041 (CDR1 residues 31-35; CDR2 residues |
| | | 50-65; CDR3 residues 98-113) |
| 1824-1923 | Protein | Sequences for superhumanized and affinity-engineered variable light domains |
| | | (L1.1-L1.104) from anti-TL1A clone 041 (CDR1 residues 24-34; CDR2 residues |
| | | 50-56; CDR3 residues 89-97) |
| 1924-1926 | Protein | Variable domain and associated CDR sequences for illustrative superhumanized |
| | | and affinity-optimized 041 VH/VL pairs |
| 1927-2132 | Protein | Sequences for affinity-engineered variable heavy domain (H1.1-H1.206) from |
| | | anti-IL23 clone IL23-A (CDR1 residues 31-35; CDR2 residues 50-66; CDR3 |
| | | residues 99-109) |
| 2133-2232 | Protein | Sequences for affinity-engineered variable light domain (L1.1-L1.117) from anti- |
| | | IL23 clone IL23-A (CDR1 residues 24-34; CDR2 residues 50-56; CDR3 residues |
| | | 89-97) |
| 2233-2239 | Protein | Sequences for illustrative affinity-optimized IL23-A VH/VL pairs |
| 2240-2271 | Protein | Sequences in Table 44 and skipped sequences |
| 2272-2311 | Protein | Illustrative sequences for TL1A × IL23 bsAbs in the 1 + 1 OrthoFab format |
| 2312-2315 | Protein | Illustrative sequences for TL1A × IL23 bsAbs in CrossMab-VH-VL charge-swap |
| | | 1 + 1 format |
| 2316-2330 | Protein | Sequences for superhumanized and affinity-engineered variable heavy domains |
| | | (H1.1-H1.486) from anti-TL1A clone 041 (CDR1 residues 31-35; CDR2 residues |
| | | 50-65; CDR3 residues 98-113) |
| 2331 | Protein | Comp18 TL1A variable light domain |
| 2332 | Protein | Comp18 TL1A variable heavy domain A |
| 2333 | Protein | Comp18 TL1A variable heavy domain B |
| 2334 | Protein | Comp18 TL1A variable heavy domain C |
| 2335 | Protein | Comp18 TL1A variable heavy domain D |
| 2336 | Protein | Comp18 TL1A variable heavy domain E |
| 2337 | Protein | Comp18 TL1A variable heavy domain F |
| 2338 | Protein | Comp18 TL1A variable heavy domain G |
| 2339 | Protein | Comp18 TL1A variable heavy domain H |
| 2340 | Protein | Comp18 TL1A variable heavy domain I |
| 2341 | Protein | Comp18 TL1A variable heavy domain J |
| 2342-2343 | Protein | Comp 19 TL1A VH/VL pair |
| 2344-2345 | Protein | Comp20 TL1A VH/VL pair |
| 2346-2347 | Protein | Comp21 TL1A VH/VL pair |
| 2348-2349 | Protein | Comp22 TL1A VH/VL pair |
| 2350-2351 | Protein | Comp23 TL1A VH/VL pair |
| 2352-2353 | Protein | Comp24 TL1A VH/VL pair |
| 2354-2355 | Protein | Comp25 TL1A VH/VL pair |
| 2356-2357 | Protein | Comp26 TL1A VH/VL pair |
| 2358-2359 | Protein | Comp27 TL1A VH/VL pair |
| 2360-2361 | Protein | Comp28 TL1A VH/VL pair |
| 2362-2363 | Protein | Comp29 TL1A VH/VL pair |
| 2364-2365 | Protein | Comp30 IL23p19 VH/VL pair |
| 2366-2367 | Protein | Comp31 IL23p19 VH/VL pair |
| 2368-2369 | Protein | Comp32 IL23p19 VH/VL pair |
| 2370-2371 | Protein | Comp33 IL23p19 VH/VL pair |
| 2372-2373 | Protein | Comp34 IL23p19 VH/VL pair |
| 2374-2375 | Protein | Comp35 IL23p19 VH/VL pair |
| 2376-2377 | Protein | Comp36 IL23p19 VH/VL pair |
| 2378-2379 | Protein | Comp37 IL23p19 VH/VL pair |

### EXAMPLES

### Example 1: TL1A Binding Domains

### 1A: Novel TL1A Binding Domains

Novel mouse and rat anti-human TL1A binding domains were generated utilizing a single plasma B-cell antibody discovery platform from Single Cell Technologies. The binding domains were then humanized using string content optimization (see, e.g., U.S. Pat. No. 7,657,380). Exemplary humanized TL1A binding domains along with their CDRs are depicted in Tables 7-10.

### 1B: Superhumanized and Affinity Enhanced TL1A Binding Domains

As will be further described in Example 5, anti-TL1A clone 041 was engineered for enhanced affinity and superhumanized. Affinity maturation was performed using a phage display high-throughput screening approach in which a library of antibody variants was subjected to iterative binding selections to enrich for higher-affinity binders. Next-generation sequencing (NGS) was used to tabulate variant frequencies before and after selection, enabling calculation of enrichment scores based on changes in relative abundance. Variants exhibiting positive enrichment were identified as candidates with improved binding properties, and enrichment calculations were optionally integrated with protein language model-derived scores to further prioritize beneficial substitutions. Superhumanization to reduce potential immunogenicity was performed mostly as in Bernett et al. (2010) J. Mol. Biol. 396(5):1474-1490 with some modifications to incorporate HLA loading probabilities from MixMHC2pred (Racle et al. (2023) Immunity 56(6):1359-1375) for identifying potentially immunogenic peptide sequences for further engineering. Sequences for novel engineered VH and VL variants are provided in SEQ ID NOS: 1427-1823 and 2316-2330 (VH) and 1824-1923 (VL). Sequences for superhumanized and affinity-engineered variable heavy domain from anti-TL1A clone 041 are provided in SEQ ID NOS: 1427-1823 and 2316-2330. It should be noted that the variable heavy domains can be paired with any of the 041 variable light domains. Sequences for superhumanized and affinity-engineered variable light domain from anti-TL1A clone 041 are provided in SEQ ID NOS: 1824-1923. It should be noted that the variable heavy domains can be paired with any of the 041 variable heavy domains. Sequences for exemplary engineered VH/VL pairs are depicted in Table 22. Table 22 depicts the sequences for illustrative superhumanized and affinity-optimized 041 VH/VL pairs. It should be noted that these pairs may be formatted as Fabs or as scFvs. Any of the VH variants may further include Q39E or Q39K substitutions, and any of the VL variants may further include Q38E or Q38K substitutions for use, e.g., in OrthoFab or charge-swap formats. Superhumanization substitutions include A120Q (Kabat) in the VL region framework 4. VH substitutions that contributed to improved TL1A binding include S53Y and H100V (Kabat) respectively in HCDR2 and HCDR3.

TABLE 22
041_H1.324_L1.27

| | Sequence | SEQ ID NO: |
|---|---|---|
| Variable heavy | QVQLVQSGAEVKKPGASVKVSCKVSGFPVTSYNVHWVRQAPGQGLEWMGVM | 1661 |
| (vh) domain | WYGGNTDYNSKFQGRVTMTRDTSTNTVYMELSSLRSEDTAVYYCARAGRYYH | |
| | DSNYYWDFPYWGQGTLVTVSS | |
| vhCDR1 | SYNVH | 102 |
| vhCDR2 | VMWYGGNTDYNSKFQG | 1924 |
| vhCDR3 | AGRYYHDSNYYWDFPY | 104 |
| Variable light | DIQMTQSPSSVSASVGDRVTITCRASEDIYSGLAWYQQKPGKSPKLLIYGASSLQ | 1204 |
| (vl) domain | DGVPSRFSGSGSGTDYTLTISSLQPEDFATYFCQEGLKSPYTFGQGTKLEIK | |
| VlCDR1 | RASEDIYSGLA | 106 |
| VlCDR2 | GASSLQD | 107 |
| VlCDR3 | QEGLKSPYT | 108 |

041_H1.447_L1.27

| | Sequence | SEQ ID NO: |
|---|---|---|
| Variable heavy | QVQLVQSGAEVKKPGASVKVSCKVSGFSVTRYNVHWVRQAPGQGLEWMGVM | 1200 |
| (vh) domain | WSGANTDYNSKFQGRVTMTRDTSTNTVYMELSSLRSEDTAVYYCARAGRYYVD | |
| | SNYYWDFPYWGQGTLVTVSS | |
| vhCDR1 | RYNVH | 1201 |
| vhCDR2 | VMWSGANTDYNSKFQG | 1202 |
| vhCDR3 | AGRYYVDSNYYWDFPY | 1203 |
| Variable light | DIQMTQSPSSVSASVGDRVTITCRASEDIYSGLAWYQQKPGKSPKLLIYGASSLQ | 1204 |
| (vl) domain | DGVPSRFSGSGSGTDYTLTISSLQPEDFATYFCQEGLKSPYTFGQGTKLEIK | |
| VlCDR1 | RASEDIYSGLA | 106 |
| VlCDR2 | GASSLQD | 107 |
| VlCDR3 | QEGLKSPYT | 108 |

041_H1.337_L1.27

| | Sequence | SEQ ID NO: |
|---|---|---|
| Variable heavy | QVQLVQSGAEVKKPGASVKVSCKVSGFSVTSYNVHWVRQAPGQGLEWMGVM | 1674 |
| (vh) domain | WYGGNTDYNSKFQGRVTMTRDTSTNTVYMELSSLRSEDTAVYYCARAGRYYV | |
| | DSNYYWDFPYWGQGTLVTVSS | |
| vhCDR1 | SYNVH | 102 |
| vhCDR2 | VMWYGGNTDYNSKFQG | 1924 |
| vhCDR3 | AGRYYVDSNYYWDFPY | 1203 |
| Variable light | DIQMTQSPSSVSASVGDRVTITCRASEDIYSGLAWYQQKPGKSPKLLIYGASSLQ | 1204 |
| (vl) domain | DGVPSRFSGSGSGTDYTLTISSLQPEDFATYFCQEGLKSPYTFGQGTKLEIK | |
| VlCDR1 | RASEDIYSGLA | 106 |
| VlCDR2 | GASSLQD | 107 |
| VlCDR3 | QEGLKSPYT | 108 |

041_H1.239_L1.27

| | Sequence | SEQ ID NO: |
|---|---|---|
| Variable heavy | QVQLVQSGAEVKKPGASVKVSCKVSGFSVTSYNVHWVRQAPGQGLEWMGVM | 2323 |
| (vh) domain | WRGGNTDYNSKFQGRVTMTRDTSTNTVYMELSSLRSEDTAVYYCARAGRYYH | |
| | DSNYYWDFPYWGQGTLVTVSS | |
| vhCDR1 | SYNVH | 102 |
| vhCDR2 | VMWRGGNTDYNSKFQG | 1926 |
| vhCDR3 | AGRYYHDSNYYWDFPY | 104 |
| Variable light | DIQMTQSPSSVSASVGDRVTITCRASEDIYSGLAWYQQKPGKSPKLLIYGASSLQ | 1204 |
| (vl) domain | DGVPSRFSGSGSGTDYTLTISSLQPEDFATYFCQEGLKSPYTFGQGTKLEIK | |
| VlCDR1 | RASEDIYSGLA | 106 |
| VlCDR2 | GASSLQD | 107 |
| VlCDR3 | QEGLKSPYT | 108 |

### 1C. Additional TL1A Binding Domains

VH, VL, and CDR sequences for additional TL1A binding domains which may find use in TL1A×IL23 bispecific antibodies are depicted in Table 23 and Table 44. As noted herein and is true for every sequence herein containing CDRs, the exact identification of the CDR locations may be slightly different depending on the numbering used as is shown in Table 3, and thus included herein are not only the CDRs that are underlined but also CDRs included within the VH and VL domains using other numbering systems. Furthermore, as for all the sequences in the Tables, these VH and VL sequences can be used either in a scFv format or in a Fab format.

TABLE 23

| | |
|---|---|
| Sequence | SEQ ID NO: |

TL1a Binding Domain of XENP53387

| | | |
|---|---|---|
| Variable Heavy | QVQLVQSGAEVKKPGASVKVSCKVSGFSVTRYNVHWVRQAPGQGLE | 1200 |
| (vh) Domain | WMGVMWSGANTDYNSKFQGRVTMTRDTSTNTVYMELSSLRSEDTAV | |
| | YYCARAGRYYVDSNYYWDFPYWGQGTLVTVSS | |
| vhCDR1 | RYNVH | 1201 |
| vhCDR2 | VMWSGANTDYNSKFQG | 1202 |
| vhCDR3 | AGRYYVDSNYYWDFPY | 1203 |
| Variable Light | DIQMTQSPSSVSASVGDRVTITCRASEDIYSGLAWYQQKPGKSPKLLIYG | 1204 |
| (vl) Domain | ASSLQDGVPSRFSGSGSGTDYTLTISSLQPEDFATYFCQEGLKSPYTFGQ | |
| | GTKLEIK | |
| vlCDR1 | RASEDIYSGLA | 1205 |
| vlCDR2 | GASSLQD | 1206 |
| vlCDR3 | QEGLKSPYT | 1207 |

Comp13

| | | |
|---|---|---|
| Variable Heavy | QVQLVQSGAEVKKPGASVKVSCKASGYDFTYYGISWVRQAPGQGLEW | 1208 |
| (vh) Domain | MGWISTYNGNTHYARMLQGRVTMTTDTSTRTAYMELRSLRSDDTAVYY | |
| | CARENYYGSGAYRGGMDVWGQGTTVTVSS | |
| vhCDR1 | YYGIS | 1209 |
| vhCDR2 | WISTYNGNTHYARMLQG | 1210 |
| vhCDR3 | ENYYGSGAYRGGMDV | 1211 |
| Variable Light | EIVLTQSPATLSLSPGERATLSCRASQSVSSYLAWYQQKPGQAPRLLIYDA | 1212 |
| (vl) Domain | SNRATGIPARFSGSGSGTDFTLTISSLEPEDFAVYYCQQRSNWPWTFGQG | |
| | TKVEIK | |
| vlCDR1 | RASQSVSSYLA | 1213 |
| v1CDR2 | DASNRAT | 1214 |
| v1CDR3 | QQRSNWPWT | 1215 |

Comp14

| | | |
|---|---|---|
| Variable Heavy | QVQLVQSGAEVKKPGASVKVSCKASGFDIQDTYMHWVKQRPGQGLEW | 1216 |
| (vh) Domain | MGRIDPASGHTKYDPKFQVRVTITRDTSTSTVYLELSSLRSEDTAVYYCA | |
| | RSGGLPDVWGQGTTVTVSS | |
| vhCDR1 | DTYMH | 1217 |
| vhCDR2 | RIDPASGHTKYDPKFQV | 1218 |
| vhCDR3 | SGGLPDV | 1219 |
| Variable Light | EIVLTQSPGTLSLSPGERATLSCRASSSVSYMYWYQQKPGQAPRPLIYATS | 1220 |
| (vl) Domain | NLASGIPDRFSGSGSGTDFTLTISRLEPEDFAVYYCQQWEGNPRTFGGGT | |
| | KLEIK | |
| vlCDR1 | RASSSVSYMY | 1221 |
| vlCDR2 | ATSNLAS | 1222 |
| vlCDR3 | QQWEGNPRT | 1223 |

Comp9

| | | |
|---|---|---|
| Variable Heavy | QVQLVQSGAEVKKPGASVKVSCKASGYTFTSYDINWVRQAPGQGLEW | 1224 |
| (vh) Domain | MGWLNPNSGYTGYAQKFQGRVTMTADRSTSTAYMELSSLRSEDTAVYY | |
| | CAREVPETAAFEYWGQGTLVTVSS | |
| vhCDR1 | SYDIN | 1225 |
| vhCDR2 | WLNPNSGYTGYAQKFQG | 1226 |
| vhCDR3 | EVPETAAFEY | 1227 |
| Variable Light | QSVLTQPPSVSGAPGQRVTISCTSSSSDIGAGLGVHWYQQLPGTAPKLLIE | 1228 |
| (vl) Domain | GYYNRPSGVPDRFSGSKSGTSASLTITGLLPEDEGDYYCQSWDGTLSAL | |
| | FGGGTKLTVL | |
| vlCDR1 | TSSSSDIGAGLGVH | 1229 |
| vlCDR2 | GYYNRPS | 1230 |
| vlCDR3 | QSWDGTLSAL | 1231 |

Comp15

| | | |
|---|---|---|
| Variable Heavy | EVQLVESGGGLVKPGGSLRLSCAASGFTFSNAWMTWVRQAPGKGLEW | 1232 |
| (vh) Domain | VGRIKSKIDAGTTDYAAPVKGRFTISRDDSKNTLYLQMNSLKTEDTAVY | |
| | YCTTDLGWGENYWGQGTLVTVSS | |
| vhCDR1 | NAWMT | 1233 |
| vhCDR2 | RIKSKIDAGTTDYAAPVKG | 1234 |
| vhCDR3 | DLGWGENY | 1235 |
| Variable Light | ENVLTQSPGTLSLSPGERATLSCRASQRVSSSYLVWYQQKPGQAPRLLIY | 1236 |
| (vl) Domain | GASSRATGIPDRFSGSGSGTDFTLTISRLEPEDFAVYYCQQYGSSPYTFGQ | |
| | GTKLEIK | |
| vlCDR1 | RASQRVSSSYLV | 1237 |
| v1CDR2 | GASSRAT | 1238 |
| vlCDR3 | QQYGSSPYT | 1239 |
| Variable Heavy | EVQLVQSGAEVKKPGASVKVSCKASGYTFTGYYMHWVRQAPGQGLEW | 1240 |
| (vh) Domain | MGWINPKSGGTNYAQKFQGRVTMTRDTSISTAYMELSRLISDDTAVYY | |
| | CATGGSFDAFDVWGQGTMVTVSS | |
| vhCDR1 | GYYMH | 1241 |
| vhCDR2 | WINPKSGGTNYAQKFQG | 1242 |
| vhCDR3 | GGSFDAFDV | 1243 |
| Variable Light | DIVMTQSPSSLSASVGDRVTITCRASQSISSYLNWYQQSPGKAPKILIYGA | 1244 |
| (vl) Domain | SRLQSGVPSRFSGSGSGTDFTLAISSLQPEDFATYYCQQGDNTPFTFGPGT | |
| | KLEIK | |
| vlCDR1 | RASQSISSYLN | 1245 |
| vlCDR2 | GASRLQS | 1246 |
| vlCDR3 | QQGDNTPFT | 1247 |

It should be noted that any of the sequences described in this Example 1 can further include the G44C and A100C substitutions (Kabat numbering), for example, for improving stability in the context of scFvs.

### Example 2: IL23 Binding Domains

### 2A: Affinity Enhanced IL23 Binding Domains

As will be further described in Example 5, anti-TL23 clone IL23-A was engineered for enhanced affinity. Affinity engineering was done through rational design. Sequences for such novel VH and VL variants are provided in SEQ ID NOS: 1927-2132 (VH) and 2133-2232 (VL). Sequences for affinity-engineered variable heavy domain from anti-IL23 clone IL23-A are provided in SEQ ID NOS: 1927-2132. It should be noted that the variable heavy domains can be paired with any of the IL23-A variable light domains. Sequences for affinity-engineered variable light domain from anti-TL23 clone IL23-A are provided in SEQ ID NOS: 2133-2232. It should be noted that the variable heavy domains can be paired with any of the IL23-A variable heavy domains. Sequences for exemplary affinity-optimized IL23-A VH/VL pairs are depicted in Table 24. It should be noted that these pairs may be formatted as Fabs or as scFvs. Any of the VH variants may further include Q39E or Q39K substitutions, and any of the VL variants may further include Q38E or Q38K substitutions for use, e.g., in OrthoFab or charge-swap formats. VL substitutions that contributed to improved IL23 binding include T66V and H105Y (Kabat) respectively in LCDR2 and LCDR3.

TABLE 24

| | |
|---|---|
| sequence | SEQ ID NO: |

IL23-A_H1_L1.117

| | | |
|---|---|---|
| Variable heavy | QVQLVQSGAEVKKPGSSVKVSCKASGYTFTDQTIHWMRQAPGQGL | 179 |
| (vh) domain | EWIGYIYPRDDSPKYNENFKGKVTITADKSTSTAYMELSSLRSEDTA | |
| | VYYCAIPDRSGYAWFIYWGQGTLVTVSS | |
| vhCDR1 | DQTIH | 180 |
| vhCDR2 | YIYPRDDSPKYNENFKG | 181 |
| vhCDR3 | PDRSGYAWFIY | 182 |
| Variable light | DIQMTQSPSSLSASVGDRVTITCKASRDVAIAVAWYQQKPGKVPKLL | 1248 |
| (vl) domain | IYWASVRHTGVPSRFSGSGSRTDFTLTISSLQPEDVADYFCYQYSSYP | |
| | FTFGSGTKLEIK | |
| vlCDR1 | KASRDVAIAVA | 184 |
| vlCDR2 | WASVRHT | 1249 |
| vlCDR3 | YQYSSYPFT | 1250 |

IL23-A_H1.187_L1.116

| | | |
|---|---|---|
| Variable heavy | QVQLVQSGAEVKKPGSSVKVSCKASGYTFTDQTIHWMRQAPGQGL | 2113 |
| (vh) domain | EWIGYIYPRDDSPKYNENFKGKVTITADKSTSTAYMELSSLRSEDTAV | |
| | YYCAIPDRSGYAFFIYWGQGTLVTVSS | |
| vhCDR1 | DQTIH | 180 |
| vhCDR2 | YIYPRDDSPKYNENFKG | 181 |
| vhCDR3 | PDRSGYAFFIY | 2236 |
| Variable light | DIQMTQSPSSLSASVGDRVTITCKASRDVAIAVAWYQQKPGKVPKLLI | 2231 |
| (vl) domain | YWASQRHTGVPSRFSGSGSRTDFTLTISSLQPEDVADYFCYQYSSYPF | |
| | TFGSGTKLEIK | |
| vICDR1 | KASRDVAIAVA | 184 |
| vICDR2 | WASQRHT | 2238 |
| vlCDR3 | YQYSSYPFT | 1250 |

IL23-A_H1.41_L1.117

| | | |
|---|---|---|
| Variable heavy | QVQLVQSGAEVKKPGSSVKVSCKASGYTFTDQTLHWMRQAPGQGL | 1967 |
| (vh) domain | EWIGYIYPRDDSPKYNENFKGKVTITADKSTSTAYMELSSLRSEDTAV | |
| | YYCAIPDRSGYAWFIYWGQGTLVTVSS | |
| vhCDR1 | DQTLH | 2239 |
| vhCDR2 | YIYPRDDSPKYNENFKG | 181 |
| vhCDR3 | PDRSGYAWFIY | 182 |
| Variable light | DIQMTQSPSSLSASVGDRVTITCKASRDVAIAVAWYQQKPGKVPKLLI | 1248 |
| (vl) domain | YWASVRHTGVPSRFSGSGSRTDFTLTISSLQPEDVADYFCYQYSSYPF | |
| | TFGSGTKLEIK | |
| vlCDR1 | KASRDVAIAVA | 184 |
| vlCDR2 | WASVRHT | 1249 |
| vlCDR3 | YQYSSYPFT | 1250 |

### 2B: Additional IL23 Binding Domains

VH, VL, and CDR sequences for additional IL23 binding domains which may find use in TL1A×IL23 bispecific antibodies are depicted in Table 25 and Table 44. As noted herein and is true for every sequence herein containing CDRs, the exact identification of the CDR locations may be slightly different depending on the numbering used as is shown in Table 3, and thus included herein are not only the CDRs that are underlined but also CDRs included within the VH and VL domains using other numbering systems. Furthermore, as for all the sequences in the Tables, these VH and VL sequences can be used either in a scFv format or in a Fab format.

TABLE 25

| | |
|---|---|
| Sequence | SEQ ID NO: |

IL23 Binding Domain of XENP53387

| | | |
|---|---|---|
| Variable | QVQLVQSGAEVKKPGSSVKVSCKASGYTFTDQTIHWMRQAPGQGLEWIGYI | 179 |
| Heavy (vh) | YPRDDSPKYNENFKGKVTITADKSTSTAYMELSSLRSEDTAVYYCAIPDRSGY | |
| Domain | AWFIYWGQGTLVTVSS | |
| vhCDR1 | DQTIH | 180 |
| vhCDR2 | YIYPRDDSPKYNENFKG | 181 |
| vhCDR3 | PDRSGYAWFIY | 182 |
| Variable | DIQMTQSPSSLSASVGDRVTITCKASRDVAIAVAWYQQKPGKVPKLLIYWAS | 1248 |
| Light (v1) | VRHTGVPSRFSGSGSRTDFTLTISSLQPEDVADYFCYQYSSYPFTFGSGTKLEI | |
| Domain | K | |
| vlCDR1 | KASRDVAIAVA | 184 |
| vlCDR2 | WASVRHT | 1249 |
| vlCDR3 | YQYSSYPFT | 1250 |

Comp5

| | | |
|---|---|---|
| Variable | EVQLVQSGAEVKKPGESLKISCKGSGYSFSNYWIGWVRQMPGKGLEWMGII | 1251 |
| Heavy (vh) | DPSNSYTRYSPSFQGQVTISADKSISTAYLQWSSLKASDTAMYYCARWYYKP | |
| Domain | FDVWGQGTLVTVSS | |
| vhCDR1 | NYWIG | 1252 |
| vhCDR2 | IIDPSNSYTRYSPSFQG | 1253 |
| vhCDR3 | WYYKPFDV | 1254 |
| Variable | QSVLTQPPSVSGAPGQRVTISCTGSSSNIGSGYDVHWYQQLPGTAPKLLIYGN | 1255 |
| Light (vl) | SKRPSGVPDRFSGSKSGTSASLAITGLQSEDEADYYCASWTDGLSLVVFGGG | |
| Domain | TKLTVL | |
| vlCDR1 | TGSSSNIGSGYDVH | 1256 |
| vlCDR2 | GNSKRPS | 1257 |
| vlCDR3 | ASWTDGLSLVV | 1258 |

Comp6

| | | |
|---|---|---|
| Variable | QVQLVQSGAEVKKPGASVKVSCKASGYIFITYWMTWVRQAPGQGLEWMGQ | 1259 |
| Heavy (vh) | IFPASGSADYNEKFEGRVTMTTDTSTSTAYMELRSLRSDDTAVYYCARGGGG | |
| Domain | FAYWGQGTLVTVSS | |
| vhCDR1 | TYWMT | 1260 |
| vhCDR2 | QIFPASGSADYNEKFEG | 1261 |
| vhCDR3 | GGGGFAY | 1262 |
| Variable | DIQMTQSPSSLSASVGDRVTITCRTSENIYSYLAWYQQKPGKAPKLLIYNAKT | 1263 |
| Light (vl) | LAEGVPSRFSGSGSGTDFTLTISSLQPEDFATYYCQHHYGIPFTFGQGTKVEIK | |
| Domain | | |
| vlCDR1 | RTSENIYSYLA | 1264 |
| vlCDR2 | NAKTLAE | 1265 |
| vlCDR3 | QHHYGIPFT | 1266 |
| Variable | QVQLVQSGAEVKKPGSSVKVSCKASGYKFTRYVMHWVRQAPGQGLEWMG | 1267 |
| Heavy (vh) | YINPYNDGTNYNEKFKGRVTITADKSTSTAYMELSSLRSEDTAVYYCARNWD | |
| Domain | TGLWGQGTTVTVSS | |
| vhCDR1 | RYVMH | 1268 |
| vhCDR2 | YINPYNDGTNYNEKFKG | 1269 |
| vhCDR3 | NWDTGL | 1270 |
| Variable | DIQMTQSPSSLSASVGDRVTITCKASDHILKFLTWYQQKPGKAPKLLIYGATS | 1271 |
| Light (vl) | LETGVPSRFSGSGSGTDFTLTISSLQPEDFATYYCQMYWSTPFTFGGGTKVEIK | |
| Domain | | |
| vlCDR1 | KASDHILKFLT | 1272 |
| vlCDR2 | GATSLET | 1273 |
| vlCDR3 | QMYWSTPFT | 1274 |

Comp7

| | | |
|---|---|---|
| Variable | QVQLVESGGGVVQPGRSLRLSCAASGFTFSSYGMHWVRQAPGKGLEWVAVI | 1275 |
| Heavy (vh) | WYDGSNEYYADSVKGRFTISRDNSKNTLYLQMNSLRAEDTAVYYCARDRGY | |
| Domain | TSSWYPDAFDIWGQGTMVTVSS | |
| vhCDR1 | SYGMH | 1276 |
| vhCDR2 | VIWYDGSNEYYADSVKG | 1277 |
| vhCDR3 | DRGYTSSWYPDAFDI | 1278 |
| Variable | QSVLTQPPSVSGAPGQRVTISCTGSSSNTGAGYDVHWYQQVPGTAPKLLIYG | 1279 |
| Light (vl) | SGNRPSGVPDRFSGSKSGTSASLAITGLQAEDEADYYCQSYDSSLSGWVFGG | |
| Domain | GTRLTVL | |
| vlCDR1 | TGSSSNTGAGYDVH | 1280 |
| vlCDR2 | GSGNRPS | 1281 |
| vlCDR3 | QSYDSSLSGWV | 1282 |
| Variable | EVQLLESGGGLVQPGGSLRLSCAASGFTFSAYGMDWVRQAPGKGLEWVSSI | 1283 |
| Heavy (vh) | SPSGGRTKYADSVKGRFTISRDNSKNTLYLQMNSLRAEDTAVYYCAKDLGGG | |
| Domain | YYYYYGMDVWGQGTLVTVSS | |
| vhCDR1 | AYGMD | 1284 |
| vhCDR2 | SISPSGGRTKYADSVKG | 1285 |
| vhCDR3 | DLGGGYYYYYGMDV | 1286 |
| Variable | QYELTQPASVSGSPGQSITISCTGTSSDVGGYNYVSWYQQHPGKAPKLMIYE | 1287 |
| Light (vl) | VSNRPSGVSNRFSGSKSGNTASLTISGLQAEDEADYYCSSYTSSSTLFYVFGT | |
| Domain | GTKVTVL | |
| vlCDR1 | TGTSSDVGGYNYVS | 1288 |
| vlCDR2 | EVSNRPS | 1289 |
| vlCDR3 | SSYTSSSTLFYV | 1290 |

It should be noted that any of the sequences described in this Example 2 can further include the G44C and A100C substitutions (Kabat numbering), for example, for improving stability in the context of scFvs.

### Example 3: Engineering TL1a×IL23 bsAbs

A number of formats were conceived and engineered for the production of TL1A×IL23 bispecific antibodies which are depicted in FIG. 1. Exemplary formats are further outlined below.

### 3A: 1+1 Fab-scFv-Fc Format

One exemplary format utilizing Fab domains and scFv is the 1+1 Fab-scFv-Fc format (depicted schematically in FIG. 1, Panel A) which comprises a first monomer comprising a single-chain Fv (“scFv”) with a first antigen binding specificity covalently attached to a first heterodimeric Fc domain i.e., scFv-domain linker-CH2-CH3, a second monomer comprising a heavy chain i.e., VH-CH1-hinge-CH2-CH3, wherein the CH2-CH3 is a second heterodimeric Fc domain complementary to the first heterodimeric Fc domain, and a light chain (LC) transfected separately so that a Fab domain having a second antigen binding specificity is formed with the variable heavy domain. Any number of heterodimerization approaches as is known in the art could find use in this (and other) bispecific formats, in combination with any number of approaches for purifying heterodimers from contaminating homodimers. Any of the number of linkers as is known in the art may find use in linking the VH and VL domains of the scFv. Finally, it may be useful to maximize serum half-life of the bsAbs, and any of the number of half-life extending variants as is known in the art may find use in these bsAbs.

In particular, the 1+1 Fab-scFv-Fc bsAbs may utilize Backbones 1 (SEQ ID NOS: 58 and 58) or 11 (SEQ ID NOS: 78 and 79). The backbones utilize the L368D/K370S (on the HC): S364K/E357Q (on the scFv-Fc) heterodimeric Fc variants. The HC side further includes pI variants N208D/Q295E/N384D/Q418E/N421D to increase negative charge of the heavy chain. The scFv utilizes a positively charged (GKPGS)₄ (SEQ ID NO: 16) linker between the VH and VL domains to increase positive charge of the scFv-Fc chain. Collectively, these two approaches enable easy purification of heterodimers from contaminating homodimers. The FcγR ablation variants utilized in this platform are the E233P/L234V/L235A/G236_/S267K substitutions on both the HC and the scFv-Fc monomers. In some cases, bsAbs includes the M428L/N434S half-life extension variants. Sequences for illustrative TL1A×IL23 bsAbs in the 1+1 Fab-scFv-Fc format are provided in Table 26. CDRs are underlined and slashes indicate the border(s) between the variable regions, linkers, Fe regions, and constant domains. The scFv domain has orientation (N- to C-terminus) of V_{H}-scFv linker-V_{L}, although this can be reversed. It should be noted that the Chain 2 sequences include as a domain linker the sequence/GKPGSGKPGSGKPGSGKPGS/(SEQ ID NO: 892); however, this linker can be replaced with any domain linker include any of the “useful domain linkers” of Table 4. It should be noted that the TL1A×IL23 bsAbs can utilize variable region, Fc region, and constant domain sequences that are 90, 95, 98 and 9900 identical (as defined herein), and/or contain from 1, 2, 3, 4, 5, 6, 7, 8, 9 or 10 amino acid substitutions. In addition, each sequence outlined herein can include or exclude the M428L/N434S or any other half-life extension variants and/or any other ablation variant (as depicted in Table 1) in one or preferably both Fc domains Additionally, each sequence can utilize any other heterodimeric Fc variant as depicted in Tables 12-17.

TABLE 26

| | SEQ ID NO \| Antigen | | | | | |
|---|---|---|---|---|---|---|
| Xencor ID | Chain 1 | | Chain 2 | | Chain 3 | |
| XENP48732 | 186 | IL23 | 187 | TL1A | 188 | IL23 |
| XENP48733 | 189 | IL23 | 190 | TL1A | 191 | IL23 |
| XENP48734 | 192 | IL23 | 193 | TL1A | 194 | IL23 |
| XENP48735 | 195 | IL23 | 196 | TL1A | 197 | IL23 |
| XENP48736 | 198 | IL23 | 199 | TL1A | 200 | IL23 |
| XENP48737 | 201 | IL23 | 202 | TL1A | 203 | IL23 |
| XENP48738 | 204 | IL23 | 205 | TL1A | 206 | IL23 |
| XENP48739 | 207 | IL23 | 208 | TL1A | 209 | IL23 |
| XENP48740 | 210 | IL23 | 211 | TL1A | 212 | IL23 |
| XENP48741 | 213 | IL23 | 214 | TL1A | 215 | IL23 |
| XENP48742 | 216 | IL23 | 217 | TL1A | 218 | IL23 |
| XENP48743 | 219 | IL23 | 220 | TL1A | 221 | IL23 |
| XENP48744 | 222 | TL1A | 223 | IL23 | 224 | TL1A |
| XENP48745 | 225 | TL1A | 226 | IL23 | 227 | TL1A |
| XENP48746 | 228 | TL1A | 229 | IL23 | 230 | TL1A |
| XENP48747 | 231 | TL1A | 232 | IL23 | 233 | TL1A |
| XENP48748 | 234 | TL1A | 235 | IL23 | 236 | TL1A |
| XENP48749 | 237 | TL1A | 238 | IL23 | 239 | TL1A |
| XENP48750 | 240 | TL1A | 241 | IL23 | 242 | TL1A |
| XENP48751 | 243 | TL1A | 244 | IL23 | 245 | TL1A |
| XENP48752 | 246 | TL1A | 247 | IL23 | 248 | TL1A |
| XENP48753 | 249 | TL1A | 250 | IL23 | 251 | TLIA |
| XENP48754 | 252 | TL1A | 253 | IL23 | 254 | TLIA |
| XENP48755 | 255 | TLIA | 256 | IL23 | 257 | TLIA |
| XENP49328 | 258 | IL23 | 259 | TL1A | 260 | IL23 |
| XENP49329 | 261 | IL23 | 262 | TL1A | 263 | IL23 |
| XENP49330 | 264 | IL23 | 265 | TL1A | 266 | IL23 |
| XENP49331 | 267 | IL23 | 268 | TL1A | 269 | IL23 |
| XENP49332 | 270 | IL23 | 271 | TL1A | 272 | IL23 |
| XENP49333 | 273 | IL23 | 274 | TL1A | 275 | IL23 |
| XENP49334 | 276 | TLIA | 277 | IL23 | 278 | TL1A |
| XENP49335 | 279 | TLIA | 280 | IL23 | 281 | TL1A |
| XENP49336 | 282 | TL1A | 283 | IL23 | 284 | TL1A |
| XENP49337 | 285 | TL1A | 286 | IL23 | 287 | TLIA |
| XENP49338 | 288 | TL1A | 289 | IL23 | 290 | TLIA |
| XENP49339 | 291 | TL1A | 292 | IL23 | 293 | TLIA |
| XENP49699 | 294 | IL23 | 295 | TL1A | 296 | IL23 |
| XENP49700 | 297 | IL23 | 298 | TL1A | 299 | IL23 |
| XENP49701 | 300 | IL23 | 301 | TL1A | 302 | IL23 |
| XENP49702 | 303 | IL23 | 304 | TL1A | 305 | IL23 |
| XENP49703 | 306 | IL23 | 307 | TL1A | 308 | IL23 |
| XENP49704 | 309 | IL23 | 310 | TL1A | 311 | IL23 |
| XENP50284 | 312 | IL23 | 313 | TL1A | 314 | IL23 |
| XENP50774 | 315 | TL1A | 316 | IL23 | 317 | TL1A |
| XENP50776 | 318 | IL23 | 319 | TL1A | 320 | IL23 |
| XENP51725 | 321 | IL23 | 322 | TL1A | 323 | IL23 |
| XENP52371 | 324 | TL1A | 325 | IL23 | 326 | TL1A |
| XENP52372 | 327 | TL1A | 328 | IL23 | 329 | TL1A |
| XENP52373 | 330 | TL1A | 331 | IL23 | 332 | TL1A |
| XENP52374 | 333 | TL1A | 334 | IL23 | 335 | TL1A |
| XENP52375 | 336 | TL1A | 337 | IL23 | 338 | TL1A |
| XENP52376 | 339 | TL1A | 340 | IL23 | 341 | TL1A |
| XENP52377 | 342 | TL1A | 343 | IL23 | 344 | TL1A |
| XENP52378 | 345 | TL1A | 346 | IL23 | 347 | TL1A |
| XENP52379 | 348 | TL1A | 349 | IL23 | 350 | TL1A |
| XENP52380 | 351 | TL1A | 352 | IL23 | 353 | TL1A |
| XENP52381 | 354 | TL1A | 355 | IL23 | 356 | TL1A |
| XENP52382 | 357 | TL1A | 358 | IL23 | 359 | TL1A |
| XENP52383 | 360 | TL1A | 361 | IL23 | 362 | TL1A |
| XENP52384 | 363 | TL1A | 364 | IL23 | 365 | TL1A |
| XENP52385 | 366 | TL1A | 367 | IL23 | 368 | TL1A |
| XENP52386 | 369 | TLIA | 370 | IL23 | 371 | TL1A |
| XENP52387 | 372 | TL1A | 373 | IL23 | 374 | TL1A |
| XENP52388 | 375 | TL1A | 376 | IL23 | 377 | TL1A |
| XENP52389 | 378 | TL1A | 379 | IL23 | 380 | TL1A |
| XENP52390 | 381 | TL1A | 382 | IL23 | 383 | TL1A |
| XENP52391 | 384 | TL1A | 385 | IL23 | 386 | TL1A |
| XENP52392 | 387 | TL1A | 388 | IL23 | 389 | TL1A |
| XENP52393 | 390 | TL1A | 391 | IL23 | 392 | TL1A |
| XENP52394 | 393 | TL1A | 394 | IL23 | 395 | TL1A |
| XENP52395 | 396 | TL1A | 397 | IL23 | 398 | TL1A |
| XENP52396 | 399 | TL1A | 400 | IL23 | 401 | TL1A |
| XENP52432 | 402 | IL23 | 403 | TL1A | 404 | IL23 |
| XENP52433 | 405 | IL23 | 406 | TL1A | 407 | IL23 |
| XENP52438 | 408 | TL1A | 409 | IL23 | 410 | TL1A |
| XENP52439 | 411 | TL1A | 412 | IL23 | 413 | TLIA |
| XENP52440 | 414 | TL1A | 415 | IL23 | 416 | TL1A |
| XENP52441 | 417 | TL1A | 418 | IL23 | 419 | TL1A |
| XENP52442 | 420 | TL1A | 421 | IL23 | 422 | TL1A |
| XENP52443 | 423 | TL1A | 424 | IL23 | 425 | TL1A |
| XENP52444 | 426 | TL1A | 427 | IL23 | 428 | TL1A |
| XENP52445 | 429 | TL1A | 430 | IL23 | 431 | TL1A |
| XENP52446 | 432 | TL1A | 433 | IL23 | 434 | TL1A |
| XENP52447 | 435 | TL1A | 436 | IL23 | 437 | TL1A |
| XENP52448 | 438 | TL1A | 439 | IL23 | 440 | TL1A |
| XENP52449 | 441 | TLIA | 442 | IL23 | 443 | TL1A |
| XENP52450 | 444 | TL1A | 445 | IL23 | 446 | TLIA |
| XENP52451 | 447 | TL1A | 448 | IL23 | 449 | TL1A |
| XENP52452 | 450 | TL1A | 451 | IL23 | 452 | TL1A |
| XENP52453 | 453 | TL1A | 454 | IL23 | 455 | TL1A |
| XENP52454 | 456 | TL1A | 457 | IL23 | 458 | TL1A |
| XENP52455 | 459 | TL1A | 460 | IL23 | 461 | TL1A |
| XENP52456 | 462 | TL1A | 463 | IL23 | 464 | TL1A |
| XENP52457 | 465 | TL1A | 466 | IL23 | 467 | TL1A |
| XENP52458 | 468 | TL1A | 469 | IL23 | 470 | TL1A |
| XENP52459 | 471 | TL1A | 472 | IL23 | 473 | TL1A |
| XENP52460 | 474 | TL1A | 475 | IL23 | 476 | TL1A |
| XENP52461 | 477 | TL1A | 478 | IL23 | 479 | TL1A |
| XENP52462 | 480 | TL1A | 481 | IL23 | 482 | TL1A |
| XENP52463 | 483 | TL1A | 484 | IL23 | 485 | TL1A |
| XENP52464 | 486 | TL1A | 487 | IL23 | 488 | TL1A |
| XENP52465 | 489 | TL1A | 490 | IL23 | 491 | TL1A |
| XENP52466 | 492 | TL1A | 493 | IL23 | 494 | TL1A |
| XENP52467 | 495 | TL1A | 496 | IL23 | 497 | TL1A |
| XENP52468 | 498 | TL1A | 499 | IL23 | 500 | TL1A |
| XENP52469 | 501 | TL1A | 502 | IL23 | 503 | TL1A |
| XENP52470 | 504 | TL1A | 505 | IL23 | 506 | TL1A |
| XENP52471 | 507 | TL1A | 508 | IL23 | 509 | TL1A |
| XENP52472 | 510 | TL1A | 511 | IL23 | 512 | TL1A |
| XENP52473 | 513 | TL1A | 514 | IL23 | 515 | TL1A |
| XENP52474 | 516 | TL1A | 517 | IL23 | 518 | TL1A |
| XENP52475 | 519 | TL1A | 520 | IL23 | 521 | TL1A |
| XENP52476 | 522 | TL1A | 523 | IL23 | 524 | TL1A |
| XENP52477 | 525 | TL1A | 526 | IL23 | 527 | TL1A |
| XENP52478 | 528 | TL1A | 529 | IL23 | 530 | TL1A |
| XENP52479 | 531 | TL1A | 532 | IL23 | 533 | TL1A |
| XENP52480 | 534 | TL1A | 535 | IL23 | 536 | TL1A |
| XENP52481 | 537 | TL1A | 538 | IL23 | 539 | TL1A |
| XENP52482 | 540 | TL1A | 541 | IL23 | 542 | TL1A |
| XENP52483 | 543 | TL1A | 544 | IL23 | 545 | TL1A |
| XENP52484 | 546 | TL1A | 547 | IL23 | 548 | TL1A |
| XENP52485 | 549 | TL1A | 550 | IL23 | 551 | TL1A |
| XENP52486 | 552 | TL1A | 553 | IL23 | 554 | TL1A |
| XENP52487 | 555 | TL1A | 556 | IL23 | 557 | TL1A |
| XENP52488 | 558 | TL1A | 559 | IL23 | 560 | TL1A |
| XENP52489 | 561 | TL1A | 562 | IL23 | 563 | TL1A |
| XENP52490 | 564 | TL1A | 565 | IL23 | 566 | TL1A |
| XENP52491 | 567 | TL1A | 568 | IL23 | 569 | TL1A |
| XENP52492 | 570 | TL1A | 571 | IL23 | 572 | TL1A |
| XENP52493 | 573 | TL1A | 574 | IL23 | 575 | TL1A |
| XENP52494 | 576 | TL1A | 577 | IL23 | 578 | TL1A |
| XENP52495 | 579 | TL1A | 580 | IL23 | 581 | TL1A |
| XENP52496 | 582 | TL1A | 583 | IL23 | 584 | TL1A |
| XENP52497 | 585 | TL1A | 586 | IL23 | 587 | TL1A |
| XENP52498 | 588 | TL1A | 589 | IL23 | 590 | TL1A |
| XENP52499 | 591 | TL1A | 592 | IL23 | 593 | TL1A |
| XENP52500 | 594 | TL1A | 595 | IL23 | 596 | TL1A |
| XENP52501 | 597 | TL1A | 598 | IL23 | 599 | TL1A |
| XENP52502 | 600 | TL1A | 601 | IL23 | 602 | TL1A |
| XENP52503 | 603 | TL1A | 604 | IL23 | 605 | TL1A |
| XENP52504 | 606 | TL1A | 607 | IL23 | 608 | TL1A |
| XENP52505 | 609 | TL1A | 610 | IL23 | 611 | TL1A |
| XENP52506 | 612 | TL1A | 613 | IL23 | 614 | TL1A |
| XENP52507 | 615 | TL1A | 616 | IL23 | 617 | TL1A |
| XENP52508 | 618 | TL1A | 619 | IL23 | 620 | TL1A |
| XENP52509 | 621 | TL1A | 622 | IL23 | 623 | TL1A |
| XENP52510 | 624 | TL1A | 625 | IL23 | 626 | TL1A |
| XENP52511 | 627 | TL1A | 628 | IL23 | 629 | TL1A |
| XENP52528 | 630 | TL1A | 631 | IL23 | 632 | TL1A |
| XENP52529 | 633 | TL1A | 634 | IL23 | 635 | TL1A |
| XENP52530 | 636 | TL1A | 637 | IL23 | 638 | TL1A |
| XENP52531 | 639 | TL1A | 640 | IL23 | 641 | TL1A |
| XENP52532 | 642 | TL1A | 643 | IL23 | 644 | TL1A |
| XENP52533 | 645 | TL1A | 646 | IL23 | 647 | TL1A |
| XENP52534 | 648 | TL1A | 649 | IL23 | 650 | TL1A |
| XENP52535 | 651 | TL1A | 652 | IL23 | 653 | TL1A |
| XENP52536 | 654 | TL1A | 655 | IL23 | 656 | TL1A |
| XENP52537 | 657 | TLIA | 658 | IL23 | 659 | TL1A |
| XENP52538 | 660 | TL1A | 661 | IL23 | 662 | TL1A |
| XENP52850 | 663 | IL23 | 664 | TL1A | 665 | IL23 |
| XENP52851 | 666 | IL23 | 667 | TL1A | 668 | IL23 |
| XENP52852 | 669 | IL23 | 670 | TL1A | 671 | IL23 |
| XENP52853 | 672 | IL23 | 673 | TL1A | 674 | IL23 |
| XENP52854 | 675 | IL23 | 676 | TL1A | 677 | IL23 |
| XENP53110 | 678 | IL23 | 679 | TL1A | 680 | IL23 |
| XENP53111 | 681 | IL23 | 682 | TL1A | 683 | IL23 |
| XENP53112 | 684 | IL23 | 685 | TL1A | 686 | IL23 |
| XENP53113 | 687 | IL23 | 688 | TL1A | 689 | IL23 |
| XENP53114 | 690 | IL23 | 691 | TL1A | 692 | IL23 |
| XENP53115 | 693 | IL23 | 694 | TL1A | 695 | IL23 |
| XENP53116 | 696 | IL23 | 697 | TL1A | 698 | IL23 |
| XENP53117 | 699 | IL23 | 700 | TL1A | 701 | IL23 |
| XENP53118 | 702 | IL23 | 703 | TL1A | 704 | IL23 |
| XENP53119 | 705 | IL23 | 706 | TL1A | 707 | IL23 |
| XENP53194 | 708 | IL23 | 709 | TL1A | 710 | IL23 |
| XENP53195 | 711 | IL23 | 712 | TL1A | 713 | IL23 |
| XENP53196 | 714 | IL23 | 715 | TL1A | 716 | IL23 |
| XENP53197 | 717 | IL23 | 718 | TL1A | 719 | IL23 |
| XENP53198 | 720 | IL23 | 721 | TL1A | 722 | IL23 |
| XENP53199 | 723 | IL23 | 724 | TL1A | 725 | IL23 |
| XENP53200 | 726 | IL23 | 727 | TL1A | 728 | IL23 |
| XENP53201 | 729 | IL23 | 730 | TL1A | 731 | IL23 |
| XENP53202 | 732 | IL23 | 733 | TL1A | 734 | IL23 |
| XENP53203 | 735 | IL23 | 736 | TL1A | 737 | IL23 |
| XENP53204 | 738 | IL23 | 739 | TL1A | 740 | IL23 |
| XENP53205 | 741 | IL23 | 742 | TL1A | 743 | IL23 |
| XENP53206 | 744 | IL23 | 745 | TL1A | 746 | IL23 |
| XENP53207 | 747 | IL23 | 748 | TL1A | 749 | IL23 |
| XENP53208 | 750 | IL23 | 751 | TL1A | 752 | IL23 |
| XENP53209 | 753 | IL23 | 754 | TL1A | 755 | IL23 |
| XENP53210 | 756 | IL23 | 757 | TL1A | 758 | IL23 |
| XENP53211 | 759 | IL23 | 760 | TL1A | 761 | IL23 |
| XENP53212 | 762 | IL23 | 763 | TL1A | 764 | IL23 |
| XENP53213 | 765 | IL23 | 766 | TL1A | 767 | IL23 |
| XENP53214 | 768 | IL23 | 769 | TL1A | 770 | IL23 |
| XENP53215 | 771 | IL23 | 772 | TL1A | 773 | IL23 |
| XENP53216 | 774 | IL23 | 775 | TL1A | 776 | IL23 |
| XENP53217 | 777 | IL23 | 778 | TL1A | 779 | IL23 |
| XENP53218 | 780 | IL23 | 781 | TL1A | 782 | IL23 |
| XENP53219 | 783 | IL23 | 784 | TL1A | 785 | IL23 |
| XENP53220 | 786 | IL23 | 787 | TL1A | 788 | IL23 |
| XENP53221 | 789 | IL23 | 790 | TL1A | 791 | IL23 |
| XENP53222 | 792 | IL23 | 793 | TL1A | 794 | IL23 |
| XENP53223 | 795 | IL23 | 796 | TL1A | 797 | IL23 |
| XENP53224 | 798 | IL23 | 799 | TL1A | 800 | IL23 |
| XENP53225 | 801 | IL23 | 802 | TL1A | 803 | IL23 |
| XENP53226 | 804 | IL23 | 805 | TL1A | 806 | IL23 |
| XENP53227 | 807 | IL23 | 808 | TL1A | 809 | IL23 |
| XENP53228 | 810 | IL23 | 811 | TL1A | 812 | IL23 |
| XENP53229 | 813 | IL23 | 814 | TL1A | 815 | IL23 |
| XENP53230 | 816 | IL23 | 817 | TL1A | 818 | IL23 |
| XENP53231 | 819 | IL23 | 820 | TL1A | 821 | IL23 |
| XENP53232 | 822 | IL23 | 823 | TL1A | 824 | IL23 |
| XENP53233 | 825 | IL23 | 826 | TL1A | 827 | IL23 |
| XENP53234 | 828 | IL23 | 829 | TL1A | 830 | IL23 |
| XENP53235 | 831 | IL23 | 832 | TL1A | 833 | IL23 |
| XENP53236 | 834 | IL23 | 835 | TL1A | 836 | IL23 |
| XENP53237 | 837 | IL23 | 838 | TL1A | 839 | IL23 |
| XENP53238 | 840 | IL23 | 841 | TL1A | 842 | IL23 |
| XENP53239 | 843 | IL23 | 844 | TL1A | 845 | IL23 |
| XENP53240 | 846 | IL23 | 847 | TL1A | 848 | IL23 |
| XENP53241 | 849 | IL23 | 850 | TL1A | 851 | IL23 |
| XENP53242 | 852 | IL23 | 853 | TL1A | 854 | IL23 |
| XENP53243 | 855 | IL23 | 856 | TL1A | 857 | IL23 |
| XENP53244 | 858 | IL23 | 859 | TL1A | 860 | IL23 |
| XENP53245 | 861 | IL23 | 862 | TL1A | 863 | IL23 |
| XENP53246 | 864 | IL23 | 865 | TL1A | 866 | IL23 |
| XENP53247 | 867 | IL23 | 868 | TL1A | 869 | IL23 |
| XENP53248 | 870 | IL23 | 871 | TL1A | 872 | IL23 |
| XENP53249 | 873 | IL23 | 874 | TL1A | 875 | IL23 |
| XENP53250 | 876 | IL23 | 877 | TL1A | 878 | IL23 |
| XENP53251 | 879 | IL23 | 880 | TL1A | 881 | IL23 |
| XENP53252 | 882 | IL23 | 883 | TL1A | 884 | IL23 |
| XENP53253 | 885 | IL23 | 886 | TL1A | 887 | IL23 |
| XENP53254 | 888 | IL23 | 889 | TL1A | 890 | IL23 |
| XENP53255 | 891 | IL23 | 892 | TL1A | 893 | IL23 |
| XENP53256 | 894 | IL23 | 895 | TL1A | 896 | IL23 |
| XENP53257 | 897 | IL23 | 898 | TL1A | 899 | IL23 |
| XENP53258 | 900 | IL23 | 901 | TL1A | 902 | IL23 |
| XENP53259 | 903 | IL23 | 904 | TL1A | 905 | IL23 |
| XENP53260 | 906 | IL23 | 907 | TL1A | 908 | IL23 |
| XENP53261 | 909 | IL23 | 910 | TL1A | 911 | IL23 |
| XENP53262 | 912 | IL23 | 913 | TL1A | 914 | IL23 |
| XENP53263 | 915 | IL23 | 916 | TL1A | 917 | IL23 |
| XENP53282 | 918 | IL23 | 919 | TL1A | 920 | IL23 |
| XENP53283 | 921 | IL23 | 922 | TL1A | 923 | IL23 |
| XENP53284 | 924 | IL23 | 925 | TL1A | 926 | IL23 |
| XENP53285 | 927 | IL23 | 928 | TL1A | 929 | IL23 |
| XENP53286 | 930 | IL23 | 931 | TL1A | 932 | IL23 |
| XENP53287 | 933 | IL23 | 934 | TL1A | 935 | IL23 |
| XENP53288 | 936 | IL23 | 937 | TL1A | 938 | IL23 |
| XENP53289 | 939 | IL23 | 940 | TL1A | 941 | IL23 |
| XENP53290 | 942 | IL23 | 943 | TL1A | 944 | IL23 |
| XENP53291 | 945 | IL23 | 946 | TL1A | 947 | IL23 |
| XENP53292 | 948 | IL23 | 949 | TL1A | 950 | IL23 |
| XENP53293 | 951 | IL23 | 952 | TL1A | 953 | IL23 |
| XENP53294 | 954 | IL23 | 955 | TL1A | 956 | IL23 |
| XENP53295 | 957 | IL23 | 958 | TL1A | 959 | IL23 |
| XENP53296 | 960 | IL23 | 961 | TL1A | 962 | IL23 |
| XENP53297 | 963 | IL23 | 964 | TL1A | 965 | IL23 |
| XENP53298 | 966 | IL23 | 967 | TL1A | 968 | IL23 |
| XENP53299 | 969 | IL23 | 970 | TL1A | 971 | IL23 |
| XENP53300 | 972 | IL23 | 973 | TL1A | 974 | IL23 |
| XENP53301 | 975 | IL23 | 976 | TL1A | 977 | IL23 |
| XENP53302 | 978 | IL23 | 979 | TL1A | 980 | IL23 |
| XENP53303 | 981 | IL23 | 982 | TL1A | 983 | IL23 |
| XENP53304 | 984 | IL23 | 985 | TL1A | 986 | IL23 |
| XENP53305 | 987 | IL23 | 988 | TL1A | 989 | IL23 |
| XENP53306 | 990 | IL23 | 991 | TL1A | 992 | IL23 |
| XENP53307 | 993 | IL23 | 994 | TL1A | 995 | IL23 |
| XENP53317 | 996 | TL1A | 997 | IL23 | 998 | TL1A |
| XENP53318 | 999 | TL1A | 1000 | IL23 | 1001 | TL1A |
| XENP53319 | 1002 | TL1A | 1003 | IL23 | 1004 | TL1A |
| XENP53320 | 1005 | TL1A | 1006 | IL23 | 1007 | TL1A |
| XENP53321 | 1008 | TL1A | 1009 | IL23 | 1010 | TL1A |
| XENP55885 | 1011 | TL1A | 1012 | IL23 | 1013 | TL1A |
| XENP55886 | 1014 | TL1A | 1015 | IL23 | 1016 | TL1A |
| XENP55887 | 1017 | TL1A | 1018 | IL23 | 1019 | TL1A |

### 3B: OrthoFab

Production of bispecific antibodies in IgG format is challenging, as antibody heavy chains bind antibody light chains in a relatively promiscuous manner. As a result of this promiscuous pairing, concomitant expression of, e.g., two antibody heavy chains and two antibody light chains naturally leads to heavy chain homodimerization and/or scrambling of heavy chain/light chain pairings. There are numerous approaches to circumvent the problem of heavy chain homodimerization, including those described in U.S. Pat. No. 10,858,417. However, circumventing the scrambling of heavy chain/light chain pairing has been more difficult due to the complex multidomain heterodimeric interactions within antibody Fabs (i.e., assembly driven by both VH/VL and CH1/CL domain interactions).

### 3B(1): Engineering Electrostatic Variants in the CH1:CL Interface:

A first strategy is based on identification of a salt bridge formed by K213/K218 in the CH1 and D122/E123 in the CL (positions in EU numbering). Without being limited by theory, substitutions are engineered at these residues to skew towards formation of correct heavy chain/light chain pairs by an electrostatic steering mechanism. As depicted in FIG. 4, Panel A, the goal is to engineer substitutions in the CH1 and/or CL so that Heavy Chain A pairs with Light Chain A and Heavy Chain B pairs with Light Chain B by virtue of electrostatic complementarity. Conversely, as depicted in FIG. 4, Panel B, the substitutions disfavor the pairing of Heavy Chain A with Light Chain B and Heavy Chain B with Light Chain A. While FIG. 4 depicts K213E/K218D:D122K/E123K in CH1:CL interface of Fab A and WT in Fab B, additional substitution pairings as depicted in Table 27 are also contemplated. Table 27 depicts electrostatic variants which may be introduced at K213/K218:D122/E123 in the CH1:CL interface.

TABLE 27

| Fab A | | Fab B | |
|---|---|---|---|
| CH1 | CL | CH1 | CL |
| K213E/K218D | D122K/E123K | WT | WT |
| K213E/K218E | D122K/E123K | WT | WT |
| K213D/K218E | D122K/E123K | WT | WT |
| K213D/K218D | D122K/E123K | WT | WT |
| K213E/K218D | D122K/E123R | WT | WT |
| K213E/K218E | D122K/E123R | WT | WT |
| K213D/K218E | D122K/E123R | WT | WT |
| K213D/K218D | D122K/E123R | WT | WT |
| K213E/K218D | D122R/E123K | WT | WT |
| K213E/K218E | D122R/E123K | WT | WT |
| K213D/K218E | D122R/E123K | WT | WT |
| K213D/K218D | D122R/E123K | WI | WT |
| K213E/K218D | D122R/E123R | WT | WT |
| K213E/K218E | D122R/E123R | WT | WT |
| K213D/K218E | D122R/E123R | WT | WT |
| K213D/K218D | D122R/E123R | WT | WT |
| WT | WT | K213E/K218D | D122K/E123K |
| WT | WT | K213E/K218E | D122K/E123K |
| WT | WT | K213D/K218E | D122K/E123K |
| WT | WT | K213D/K218D | D122K/E123K |
| WT | WT | K213E/K218D | D122K/E123R |
| WT | WT | K213E/K218E | D122K/E123R |
| WT | WT | K213D/K218E | D122K/E123R |
| WT | WT | K213D/K218D | D122K/E123R |
| WT | WT | K213E/K218D | D122R/E123K |
| WT | WT | K213E/K218E | D122R/E123K |
| WT | WT | K213D/K218E | D122R/E123K |
| WT | WT | K213D/K218D | D122R/E123K |
| WT | WT | K213E/K218D | D122R/E123R |
| WT | WT | K213E/K218E | D122R/E123R |
| WT | WT | K213D/K218E | D122R/E123R |
| WT | WT | K213D/K218D | D122R/E123R |

### 3B(2). Engineering Steric Variants in the CH1:CL Interface:

A second strategy for developing variants described herein is based on a “knob-in-hole” pair formed by A141 in the CH1 and F116 in the CL; A141F in the CH1 and F118 in the CL; and K147 in CH1 and S131 in the CL (positions in EU numbering). Without being limited by theory, substitutions are engineered at these residues to skew towards formation of correct heavy chain/light chain pairs by a steric hindrance mechanism. As depicted in FIG. 5, Panel A, the goal is to engineer substitution in the CH1 and/or CL so that Heavy Chain A pairs with Light Chain A and Heavy Chain B pairs with Light Chain B by virtue of steric complementarity. Conversely, as depicted in FIG. 5, Panel B, the substitutions disfavor the pairing of Heavy Chain A with Light Chain B and Heavy Chain B with Light Chain A. While FIG. 5 depicts WT in Fab A and A141F:F118A in CH1:CL interface of Fab B, additional substitution pairings as depicted in Table 28 are also contemplated. Table 28 depicts electrostatic variants which may be introduced at A141:F116, A141:F118, or K147:S131 in the CH1:CL interface.

TABLE 28

| | |
|---|---|
| Fab A | Fab B |

| CH1 | CL | CH1 | CL |
|---|---|---|---|
| WT | WT | A141F | F116A |
| A141F | F116A | WT | WT |
| WT | WT | A141F | F118A |
| A141F | F118A | WT | WT |
| WT | WT | K147S | S131K |
| K147S | S131K | WT | WT |

### 3B(3). Engineering Electrostatic Variants in the VH:VL Interface:

A third strategy is based on a hydrogen bonded pair formed by Q39 in the VH and Q38 in the VL (positions in Kabat numbering). By electrostatic steering mechanism, substitutions engineered at these residues to skew towards formation of correct heavy chain/light chain pairs. As depicted in FIG. 6, Panel A, the goal is to engineer substitutions in the VH and/or VL so that Heavy Chain A pairs with Light Chain A and Heavy Chain B pairs with Light Chain B by virtue of electrostatic complementarity. Conversely, as depicted in FIG. 6, Panel B, the substitutions disfavor the pairing of Heavy Chain A with Light Chain B and Heavy Chain B with Light Chain A. While FIG. 6 depicts Q39E:Q38K in VH:VL interface of Fab A and Q39K:Q38E in VH:VL interface of Fab B, additional substitution pairings as depicted in Tables 18 and 19 are also contemplated.

### 3B(4). Combining Variants in the VH:VL and CH1:CL Interfaces:

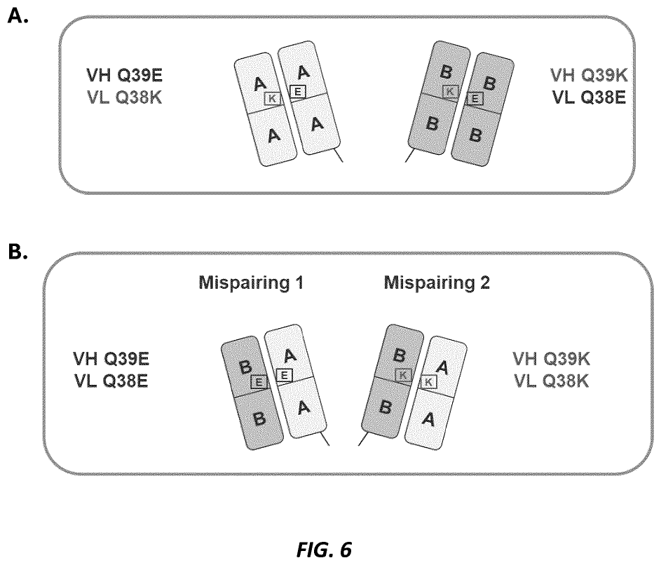

To fully leverage the multidomain interactions within the Fabs, another strategy is to combine each of the foregoing strategies, or to combine at least two of the foregoing strategies (such as, for example, VH:VL interface electrostatic variants and CH1:CL interface electrostatic variants, or VH:VL interface electrostatic variants and CH1:CL interface steric variants). As depicted in FIG. 7, Panel A, one such combination utilizes Q39E:Q38K in VH:VL interface and K213E/K218D:D122K/E123K in CH1:CL interface of Fab A and Q39K:Q38E in VH:VL interface and A141F:F118A in CH1:CL interface of Fab B; however, additional combinations of any of the substitutions in Tables 27-28 and 18-19 are also contemplated.

Schematic for OrthoFab is depicted in FIG. 1, Panel P. Sequences of illustrative TL1A×IL23 bsAbs in the OrthoFab 1+1 format including Q39E:Q38K in VH:VL interface and K213E/K218D:D122K/E123K in CH1:CL interface of Fab A and Q39K:Q38E in VH:VL interface and A141F:F118A in the CH1:CL interface is depicted in Tables 29 and 30. CDRs are underlined and slashes indicate the border(s) between the variable regions, linkers, Fc regions, and constant domains. It should be noted that the TL1A×IL23 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S or any other half-life extension variants and/or any other ablation variant (as depicted in Table 1) in one or preferably both Fc domains Additionally, each sequence can utilize any other heterodimeric Fc variant as depicted in Table 12-17.

TABLE 29
XENP49686 - IL23-A[IL23]_H1_Q39E/K213E/K218D_L1_Q38K/D122K/E123K Fab-
041[TL1a]_H1_Q39K/A141F_L1_Q38E/F118AFab-IgG1_pI(-)_Isosteric_A_PVA_/S267K/L368D/K370S-
IgG1_PVA_/S267K/S364K/E357Q
Chain 1 - IL23-A[IL23]_H1_Q39E/K213E/K218D_IgG1_pI(-)_Isosteric_A_PVA_/S267K/L368D/K370S
SEQ ID NO: 1020
QVQLVQSGAEVKKPGSSVKVSCKASGYTFTDQTIHWMREAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSDTKVDEKVEPDSCDKTHTCPPCPAPPVAGPSVFLFPPKPKDTLMISRT
PEVTCVVVDVKHEDPEVKFNWYVDGVEVHNAKTKPREEEYNSTYRVVSVLTVLHQDWLNGKEYKCKVSNKALPAPIEKTISKA
KGQPREPQVYTLPPSREEMTKNQVSLTCDVSGFYPSDIAVEWESDGQPENNYKTTPPVLDSDGSFFLYSKLTVDKSRWEQGDVFSC
SVMHEALHNHYTQKSLSLSPGK
Chain 2 - 041[TL1a]_H1_Q39K/A141F_IgG1_PVA_/S267K/S364K/E357Q
SEQ ID NO: 1021
QVQLVQSGAEVKKPGASVKVSCKVSGFSVTSYNVHWVRKAPGQGLEWMGVMWSGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAFLGCLVKDYFPEPVTVSW
NSGALTSGVHTFPAVLQSSGLYSLSSVVTVPSSSLGTQTYICNVNHKPSNTKVDKKVEPKSCDKTHTCPPCPAPPVAGPSVFLFPPK
PKDTLMISRTPEVTCVVVDVKHEDPEVKFNWYVDGVEVHNAKTKPREEQYNSTYRVVSVLTVLHQDWLNGKEYKCKVSNKALP
APIEKTISKAKGQPREPQVYTLPPSREQMTKNQVKLTCLVKGFYPSDIAVEWESNGQPENNYKTTPPVLDSDGSFFLYSKLTVDKSR
WQQGNVFSCSVMHEALHNHYTQKSLSLSPGK
Chain 3 - IL23-A[IL23]_L1_Q38K/D122K/E123K Light Chain
SEQ ID NO: 1022
DIQMTQSPSSLSASVGDRVTITCKASRDVAIAVAWYQKKPGKVPKLLIYWASTRHTGVPSRFSGSGSRTDFTLTISSLQPEDVADYF
CHQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - 041[TL1a]_L1_Q38E/F118A Light Chain
SEQ ID NO: 1023
DIQMTQSPSSVSASVGDRVTITCRASEDIYSGLAWYQEKPGKSPKLLIYGASSLQDGVPSRFSGSGSGTDYTLTISSLQPEDFATYFC
QEGLKSPYTFGAGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP49687 - IL23-A[IL23]_H1_Q39E/K213E/K218D_L1_Q38K/D122K/E123K Fab-
069[TL1a]_H1_Q39K/A141F_L1_Q38E/F118A Fab-IgG1_pI(-)_Isosteric_A_PVA_/S267K/L368D/K370S-
IgG1_PVA_/S267K/S364K/E357Q
Chain 1 - IL23-A[IL23]_H1_Q39E/K213E/K218D_IgG1_pI(-)_Isosteric_A_PVA_/S267K/L368D/K370S
SEQ ID NO: 1024
QVQLVQSGAEVKKPGSSVKVSCKASGYTFTDQTIHWMREAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSDTKVDEKVEPDSCDKTHTCPPCPAPPVAGPSVFLFPPKPKDTLMISRT
PEVTCVVVDVKHEDPEVKFNWYVDGVEVHNAKTKPREEEYNSTYRVVSVLTVLHQDWLNGKEYKCKVSNKALPAPIEKTISKA
KGQPREPQVYTLPPSREEMTKNQVSLTCDVSGFYPSDIAVEWESDGQPENNYKTTPPVLDSDGSFFLYSKLTVDKSRWEQGDVFSC
SVMHEALHNHYTQKSLSLSPGK
Chain 2 - 069[TL1a]_H1_Q39K/A141F_IgG1_PVA_/S267K/S364K/E357Q
SEQ ID NO: 1025
DIQMTQSPSSLSASVGDRVTITCKASRDVAIAVAWYQKKPGKVPKLLIYWASTRHTGVPSRFSGSGSRTDFTLTISSLQPEDVADYF
CHQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 3 - IL23-A[IL23]_L1_Q38K/D122K/E123K Light Chain
SEQ ID NO: 1026
EVQLVESGGGLVQPGGSLKLSCAASGFDIIDDYIHWVRKASGKGLEWVGRIDPASANTKYVPSVKGRFTISADTSKNTAYLQMNS
LKTEDTAVYYCTRKGFAYWGQGTLVTVSS/ASTKGPSVFPLAPSSKSTSGGTAFLGCLVKDYFPEPVTVSWNSGALTSGVHTFPAV
LQSSGLYSLSSVVTVPSSSLGTQTYICNVNHKPSNTKVDKKVEPKSCDKTHTCPPCPAPPVAGPSVFLFPPKPKDTLMISRTPEVT
CVVVDVKHEDPEVKFNWYVDGVEVHNAKTKPREEQYNSTYRVVSVLTVLHQDWLNGKEYKCKVSNKALPAPIEKTISKAKGQPRE
PQVYTLPPSREQMTKNQVKLTCLVKGFYPSDIAVEWESNGQPENNYKTTPPVLDSDGSFFLYSKLTVDKSRWQQGNVFSCSVMHE
ALHNHYTQKSLSLSPGK
Chain 4 - 069[TL1a]_L1_Q38E/F118A Light Chain
SEQ ID NO: 1027
NIVMTQTPLSLSVTPGQPASISCKASENVGTYVSWYLEKPGQSPQLLIYGTSNRYTGVPDRFTGSGSATDFTLKISRVEAEDVGVYH
CGQSYRYPYTFGGGTKLEIK/RTVAAPSVFIAPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
XENP49688 - IL23-A[IL23]_H1_Q39E/K213E/K218D_L1_Q38K/D122K/E123K Fab-
111[TL1a]_H1_Q39K/A141F_L1_Q38E/F118A Fab-IgG1_pI(-)_Isosteric_A_PVA_/S267K/L368D/K370S-
IgG1_PVA_/S267K/S364K/E357Q
Chain 1 - IL23-A[IL23]_H1_Q39E/K213E/K218D_IgG1_pI(-)_Isosteric_A_PVA_/S267K/L368D/K370S
SEQ ID NO: 1028
QVQLVQSGAEVKKPGSSVKVSCKASGYTFTDQTIHWMREAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSDTKVDEKVEPDSCDKTHTCPPCPAPPVAGPSVFLFPPKPKDTLMISRT
PEVTCVVVDVKHEDPEVKFNWYVDGVEVHNAKTKPREEEYNSTYRVVSVLTVLHQDWLNGKEYKCKVSNKALPAPIEKTISKA
KGQPREPQVYTLPPSREEMTKNQVSLTCDVSGFYPSDIAVEWESDGQPENNYKTTPPVLDSDGSFFLYSKLTVDKSRWEQGDVFSC
SVMHEALHNHYTQKSLSLSPGK
Chain 2 - 111[TL1a]_H1_Q39K/A141F_IgG1_PVA_/S267K/S364K/E357Q
SEQ ID NO: 1029
EVQLVESGGGLVQPGGSLRLSCAASGFTFSSFGMHWVRKAPGKGLEWVSYISSGSTTIYYADSVKGRFTISRDNAKNSLYLQMNS
LRAEDTAVYYCARAPLLRGAMDYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVMHEALHNHYTQKSLSLSPGK
Chain 3 - IL23-A[IL23]_L1_Q38K/D122K/E123K Light Chain
SEQ ID NO: 1030
DIQMTQSPSSLSASVGDRVTITCKASRDVAIAVAWYQKKPGKVPKLLIYWASTRHTGVPSRFSGSGSRTDFTLTISSLQPEDVADYF
CHQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - 111[TL1a]_L1_Q38E/F118A Light Chain
SEQ ID NO: 1031
DIVMTQSPDSLAVSLGERATINCKASQDVSTAVVWYQEKPGQSPKLLIYWASTRHTGVPDRFTGSGSGTDYTLTISSLQAEDVAVY
HCQQHYSTPYTFGGGTKLEIK/RTVAAPSVFIAPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
XENP49689 - IL23-A[IL23]_H1_Q39E/K213E/K218D_L1_Q38K/D122K/E123K_Fab-
041[TL1a]_H1_Q39K/A141F_L1_Q38E/F118A Fab-IgG1_pI(-)_Isosteric_A_PVA_/
S267K/L368D/K370S/M428L/N434S-IgG1_PVA_/S267K/S364K/E357Q/M428L/N434S
Chain 1 - IL23-A[IL23]_H1_Q39E/K213E/K218D_IgG1_pI(-)_Isosteric_A_PVA_/
S267K/L368D/K370S/M428L/N434S SEQ ID NO: 1032
QVQLVQSGAEVKKPGSSVKVSCKASGYTFTDQTIHWMREAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSDTKVDEKVEPDSCDKTHTCPPCPAPPVAGPSVFLFPPKPKDTLMISRT
PEVTCVVVDVKHEDPEVKFNWYVDGVEVHNAKTKPREEEYNSTYRVVSVLTVLHQDWLNGKEYKCKVSNKALPAPIEKTISKA
KGQPREPQVYTLPPSREEMTKNQVSLTCDVSGFYPSDIAVEWESDGQPENNYKTTPPVLDSDGSFFLYSKLTVDKSRWEQGDVFSC
SVLHEALHSHYTQKSLSLSPGK
Chain 2 - 041[TL1a]_H1_Q39K/A141F_IgG1_PVA_/S267K/S364K/E357Q/M428L/N434S
SEQ ID NO: 1033
QVQLVQSGAEVKKPGASVKVSCKVSGFSVTSYNVHWVRKAPGQGLEWMGVMWSGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAFLGCLVKDYFPEPVTVSW
NSGALTSGVHTFPAVLQSSGLYSLSSVVTVPSSSLGTQTYICNVNHKPSNTKVDKKVEPKSCDKTHTCPPCPAPPVAGPSVFLFPPK
PKDTLMISRTPEVTCVVVDVKHEDPEVKFNWYVDGVEVHNAKTKPREEQYNSTYRVVSVLTVLHQDWLNGKEYKCKVSNKALP
APIEKTISKAKGQPREPQVYTLPPSREQMTKNQVKLTCLVKGFYPSDIAVEWESNGQPENNYKTTPPVLDSDGSFFLYSKLTVDKSR
WQQGNVFSCSVLHEALHSHYTQKSLSLSPGK
Chain 3 - IL23-A[IL23]_L1_Q38K/D122K/E123K Light Chain
SEQ ID NO: 1034
DIQMTQSPSSLSASVGDRVTITCKASRDVAIAVAWYQKKPGKVPKLLIYWASTRHTGVPSRFSGSGSRTDFTLTISSLQPEDVADYF
CHQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - 041[TL1a]_L1_Q38E/F118A Light Chain
SEQ ID NO: 1035
DIQMTQSPSSVSASVGDRVTITCRASEDIYSGLAWYQEKPGKSPKLLIYGASSLQDGVPSRFSGSGSGTDYTLTISSLQPEDFATYFC
QEGLKSPYTFGAGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP49690 - IL23-A[IL23]_H1_Q39E/K213E/K218D_L1_Q38K/D122K/E123K Fab-
069[TL1a]_H1_Q39K/A141F_L1_Q38E/F118A Fab-IgG1_pI(-)_Isosteric_A_PVA_/
S267K/L368D/K370S/M428L/N434S-
IgG1_PVA_/S267K/S364K/E357Q/M428L/N434S
Chain - IL23-A[IL23]_H1_Q39E/K213E/K218D_IgG1_pI(-)_Isosteric_A_PVA_/
S267K/L368D/K370S/M428L/N434S SEQ ID NO: 1036
QVQLVQSGAEVKKPGSSVKVSCKASGYTFTDQTIHWMREAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSDTKVDEKVEPDSCDKTHTCPPCPAPPVAGPSVFLFPPKPKDTLMISRT
PEVTCVVVDVKHEDPEVKFNWYVDGVEVHNAKTKPREEEYNSTYRVVSVLTVLHQDWLNGKEYKCKVSNKALPAPIEKTISKA
KGQPREPQVYTLPPSREEMTKNQVSLTCDVSGFYPSDIAVEWESDGQPENNYKTTPPVLDSDGSFFLYSKLTVDKSRWEQGDVFSC
SVLHEALHSHYTQKSLSLSPGK
Chain 2 - 069[TL1a]_H1_Q39K/A141F_IgG1_PVA_/S267K/S364K/E357Q/M428L/N434S
SEQ ID NO: 1037
EVQLVESGGGLVQPGGSLKLSCAASGFDIIDDYIHWVRKASGKGLEWVGRIDPASANTKYVPSVKGRFTISADTSKNTAYLQMNS
LKTEDTAVYYCTRKGFAYWGQGTLVTVSS/ASTKGPSVFPLAPSSKSTSGGTAFLGCLVKDYFPEPVTVSWNSGALTSGVHTFPAV
LQSSGLYSLSSVVTVPSSSLGTQTYICNVNHKPSNTKVDKKVEPKSCDKTHTCPPCPAPPVAGPSVFLFPPKPKDTLMISRTPEVTCV
VVDVKHEDPEVKFNWYVDGVEVHNAKTKPREEQYNSTYRVVSVLTVLHQDWLNGKEYKCKVSNKALPAPIEKTISKAKGQPRE
PQVYTLPPSREQMTKNQVKLTCLVKGFYPSDIAVEWESNGQPENNYKTTPPVLDSDGSFFLYSKLTVDKSRWQQGNVFSCSVLHE
ALHSHYTQKSLSLSPGK
Chain 3 - IL23-A[IL23]_L1_Q38K/D122K/E123K Light Chain
SEQ ID NO: 1038
DIQMTQSPSSLSASVGDRVTITCKASRDVAIAVAWYQKKPGKVPKLLIYWASTRHTGVPSRFSGSGSRTDFTLTISSLQPEDVADYF
CHQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - 069[TL1a]_L1_Q38E/F118A Light Chain
SEQ ID NO: 1039
NIVMTQTPLSLSVTPGQPASISCKASENVGTYVSWYLEKPGQSPQLLIYGTSNRYTGVPDRFTGSGSATDFTLKISRVEAEDVGVYH
CGQSYRYPYTFGGGTKLEIK/RTVAAPSVFIAPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
XENP49691 - IL23-A[IL23]_H1_Q39E/K213E/K218D_L1_Q38K/D122K/E123K Fab-
111[TL1a]_H1_Q39K/A141F_L1_Q38E/F118AFab-IgG1_pI(-)IsostericA PVA /S267K/
L368D/K370S/M428L/N434S-
IgG1_PVA_/S267K/S364K/E357Q/M428L/N434S
Chain - IL23-A[IL23]_H1_Q39E/K213E/K218D_IgG1_pI(-)_Isosteric_A_PVA_/
S267K/L368D/K370S/M428L/N434S SEQ ID NO: 1040
QVQLVQSGAEVKKPGSSVKVSCKASGYTFTDQTIHWMREAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSDTKVDEKVEPDSCDKTHTCPPCPAPPVAGPSVFLFPPKPKDTLMISRT
PEVTCVVVDVKHEDPEVKFNWYVDGVEVHNAKTKPREEEYNSTYRVVSVLTVLHQDWLNGKEYKCKVSNKALPAPIEKTISKA
KGQPREPQVYTLPPSREEMTKNQVSLTCDVSGFYPSDIAVEWESDGQPENNYKTTPPVLDSDGSFFLYSKLTVDKSRWEQGDVFSC
SVLHEALHSHYTQKSLSLSPGK
Chain 2 - 111[TL1a]_H1_Q39K/A141F_IgG1_PVA_/S267K/S364K/E357Q/M428L/N434S
SEQ ID NO: 1041
EVQLVESGGGLVQPGGSLRLSCAASGFTFSSFGMHWVRKAPGKGLEWVSYISSGSTTIYYADSVKGRFTISRDNAKNSLYLQMNS
LRAEDTAVYYCARAPLLRGAMDYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMIS
RTPEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - IL23-A[IL23]_L1_Q38K/D122K/E123K Light Chain
SEQ ID NO: 1042
DIQMTQSPSSLSASVGDRVTITCKASRDVAIAVAWYQKKPGKVPKLLIYWASTRHTGVPSRFSGSGSRTDFTLTISSLQPEDVADYF
CHQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - 111[TL1a]_L1_Q38E/F118A Light Chain
SEQ ID NO: 1043
DIVMTQSPDSLAVSLGERATINCKASQDVSTAVVWYQEKPGQSPKLLIYWASTRHTGVPDRFTGSGSGTDYTLTISSLQAEDVAVY
HCQQHYSTPYTFGGGTKLEIK/RTVAAPSVFIAPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
XENP49746 - 041[TL1a]_H1_Q39E/K213E/K218D_L1_Q38K/D122K/E123K_Fab-IL23-
A[IL23]_H1_Q39K/A141F_L1_Q38E/F118A Fab-IgG1_pI(-)_Isosteric_A_PVA_/S267K/L368D/K370S-
IgG1_PVA_/S267K/S364K/E357Q
Chain - 041[TL1a]_H1_Q39E/K213E/K218D_IgG1_pI(-)_Isosteric_A_PVA_/S267K/L368D/K370S
SEQ ID NO: 1044
QVQLVQSGAEVKKPGASVKVSCKVSGFSVTSYNVHWVREAPGQGLEWMGVMWSGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPP
KPKDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVMHEALHNHYTQKSLSLSPGK
Chain 2 - IL23-A[IL23]_H1_Q39K/A141F_IgG1_PVA_/S267K/S364K/E357Q
SEQ ID NO: 1045
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMIS
RTPEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVMHEALHNHYTQKSLSLSPGK
Chain 3 - 041[TL1a]_L1_Q38K/D122K/E123K Light Chain
SEQ ID NO: 1046
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGAGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_L1_Q38E/F118A Light Chain
SEQ ID NO: 1047
DIQMTQSPSSLSASVGDRVTITCKASRDVAIAVAWYQEKPGKVPKLLIYWASTRHTGVPSRFSGSGSRTDFTLTISSLQPEDVADYF
CHQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP49747 - 069[TL1a]_H1_Q39E/K213E/K218D_L1_Q38K/D122K/E123K_Fab-IL23-
A[IL23]_H1_Q39K/A141F_L1_Q38E/F118A_Fab-IgG1_pI(-)_Isosteric_A_PVA_/S267K/L368D/K370S-
IgG1_PVA_/S267K/S364K/E357Q
Chain - 069[TL1a]_H1_Q39E/K213E/K218D_IgG1_pI(-)_Isosteric_A_PVA_/S267K/L368D/K370S
SEQ ID NO: 1048
EVQLVESGGGLVQPGGSLKLSCAASGFDIIDDYIHWVREASGKGLEWVGRIDPASANTKYVPSVKGRFTISADTSKNTAYLQMNSL
KTEDTAVYYCTRKGFAYWGQGTLVTVSS/ASTKGPSVFPLAPSSKSTSGGTAALGCLVKDYFPEPVTVSWNSGALTSGVHTFPAVL
QSSGLYSLSSVVTVPSSSLGTQTYICNVNHKPSDTKVDEKVEPDSCDKTHTCPPCPAPPVAGPSVFLFPPKPKDTLMISRTPEVTC
VVVDVKHEDPEVKFNWYVDGVEVHNAKTKPREEEYNSTYRVVSVLTVLHQDWLNGKEYKCKVSNKALPAPIEKTISKAKGQPREPQ
VYTLPPSREEMTKNQVSLTCDVSGFYPSDIAVEWESDGQPENNYKTTPPVLDSDGSFFLYSKLTVDKSRWEQGDVFSCSVMHEAL
HNHYTQKSLSLSPGK
Chain 2 - IL23-A[IL23]_H1_Q39K/A141F_IgG1_PVA_/S267K/S364K/E357Q
SEQ ID NO: 1049
NIVMTQTPLSLSVTPGQPASISCKASENVGTYVSWYLKKPGQSPQLLIYGTSNRYTGVPDRFTGSGSATDFTLKISRVEAEDVGVYH
CGOSYRYPYTFGGGTKLEIK/RTVAAPSVFIFPPSKKQLKSGTASVVCLLNNFYPREAKVQWKVDNALQSGNSQESVTEQDSKDST
YSLSSTLTLSKADYEKHKVYACEVTHQGLSSPVTKSFNRGEC
Chain 3 - 069[TL1a]_L1_Q38K/D122K/E123K Light Chain
SEQ ID NO: 1050
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMIS
RTPEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVMHEALHNHYTQKSLSLSPGK
Chain 4 - IL23-A[IL23]_L1_Q38E/F118A Light Chain
SEQ ID NO: 1051
DIQMTQSPSSLSASVGDRVTITCKASRDVAIAVAWYQEKPGKVPKLLIYWASTRHTGVPSRFSGSGSRTDFTLTISSLQPEDVADYF
CHQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP49748 - 111[TL1a]_H1_Q39E/K213E/K218D_L1_Q38K/D122K/E123K_Fab-IL23-
A[IL23]_H1_Q39K/A141F_L1_Q38E/F118A Fab-IgG1 pI(-)_Isosteric_A_PVA_/S267K/L368D/K370S-
IgG1_PVA_/S267K/S364K/E357Q
Chain - 111[TL1a]_H1_Q39E/K213E/K218D_IgG1_pI(-)_Isosteric_A_PVA_/S267K/L368D/K370S
SEQ ID NO: 1052
EVQLVESGGGLVQPGGSLRLSCAASGFTFSSFGMHWVREAPGKGLEWVSYISSGSTTIYYADSVKGRFTISRDNAKNSLYLQMNSL
RAEDTAVYYCARAPLLRGAMDYWGQGTLVTVSS/ASTKGPSVFPLAPSSKSTSGGTAALGCLVKDYFPEPVTVSWNSGALTSGVHT
FPAVLQSSGLYSLSSVVTVPSSSLGTQTYICNVNHKPSDTKVDEKVEPDSCDKTHTCPPCPAPPVAGPSVFLFPPKPKDTLMISRT
PEVTCVVVDVKHEDPEVKFNWYVDGVEVHNAKTKPREEEYNSTYRVVSVLTVLHQDWLNGKEYKCKVSNKALPAPIEKTISKAK
GQPREPQVYTLPPSREEMTKNQVSLTCDVSGFYPSDIAVEWESDGQPENNYKTTPPVLDSDGSFFLYSKLTVDKSRWEQGDVFSCS
VMHEALHNHYTQKSLSLSPGK
Chain 2 - IL23-A[IL23]_H1_Q39K/A141F_IgG1_PVA_/S267K/S364K/E357Q
SEQ ID NO: 1053
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMIS
RTPEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVMHEALHNHYTQKSLSLSPGK
Chain 3 - 111[TL1a]_L1_Q38K/D122K/E123K Light Chain
SEQ ID NO: 1054
DIVMTQSPDSLAVSLGERATINCKASQDVSTAVVWYQKKPGQSPKLLIYWASTRHTGVPDRFTGSGSGTDYTLTISSLQAEDVAVY
HCQQHYSTPYTFGGGTKLEIK/RTVAAPSVFIFPPSKKQLKSGTASVVCLLNNFYPREAKVQWKVDNALQSGNSQESVTEQDSKDS
TYSLSSTLTLSKADYEKHKVYACEVTHQGLSSPVTKSFNRGEC
Chain 4 - IL23-A[IL23]_L1_Q38E/F118A Light Chain
SEQ ID NO: 1055
DIQMTQSPSSLSASVGDRVTITCKASRDVAIAVAWYQEKPGKVPKLLIYWASTRHTGVPSRFSGSGSRTDFTLTISSLQPEDVADYF
CHQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP49749 - 041[TL1a]_H1_Q39E/K213E/K218D_L1_Q38K/D122K/E123K_Fab-IL23-
A[IL23]_H1_Q39K/A141F_L1_Q38E/F118A Fab-IgG1_pI(-)_Isosteric_A_PVA_/
S267K/L368D/K370S/M428L/N434S-
IgG1_PVA_/S267K/S364K/E357Q/M428L/N434S
Chain - 041[TL1a]_H1_Q39E/K213E/K218D_IgG1_pI(-)_Isosteric_A_PVA_/
S267K/L368D/K370S/M428L/N434S
SEQ ID NO: 1056
QVQLVQSGAEVKKPGASVKVSCKVSGFSVTSYNVHWVREAPGQGLEWMGVMWSGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1_Q39K/A141F_IgG1_PVA_/S267K/S364K/E357Q/M428L/N434S
SEQ ID NO: 1057
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - 041[TL1a]_L1_Q38K/D122K/E123K Light Chain
SEQ ID NO: 1058
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGAGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_L1_Q38E/F118A Light Chain
SEQ ID NO: 1059
DIQMTQSPSSLSASVGDRVTITCKASRDVAIAVAWYQEKPGKVPKLLIYWASTRHTGVPSRFSGSGSRTDFTLTISSLQPEDVADYF
CHQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP49750 - 069[TL1a]_H1_Q39E/K213E/K218D_L1_Q38K/D122K/E123K Fab-IL23-
A[IL23]_H1_Q39K/A141F_L1_Q38E/F118A_Fab-IgG1_pI(-)_Isosteric_A_PVA_/
S267K/L368D/K370S/M428L/N434S-
IgG1_PVA_/S267K/S364K/E357Q/M428L/N434S
Chain - 069[TL1a]_H1_Q39E/K213E/K218D_IgG1_pI(-)_Isosteric_A_PVA_/
S267K/L368D/K370S/M428L/N434S
SEQ ID NO: 1060
EVQLVESGGGLVQPGGSLKLSCAASGFDIIDDYIHWVREASGKGLEWVGRIDPASANTKYVPSVKGRFTISADTSKNTAYLQMNSL
KTEDTAVYYCTRKGFAYWGQGTLVTVSS/ASTKGPSVFPLAPSSKSTSGGTAALGCLVKDYFPEPVTVSWNSGALTSGVHTFPAVL
QSSGLYSLSSVVTVPSSSLGTQTYICNVNHKPSDTKVDEKVEPDSCDKTHTCPPCPAPPVAGPSVFLFPPKPKDTLMISRTPEVTCVV
VDVKHEDPEVKFNWYVDGVEVHNAKTKPREEEYNSTYRVVSVLTVLHQDWLNGKEYKCKVSNKALPAPIEKTISKAKGQPREPQ
VYTLPPSREEMTKNQVSLTCDVSGFYPSDIAVEWESDGQPENNYKTTPPVLDSDGSFFLYSKLTVDKSRWEQGDVFSCSVLHEALH
SHYTQKSLSLSPGK
Chain 2 - IL23-A[IL23]_H1_Q39K/A141F_IgG1_PVA_/S267K/S364K/E357Q/M428L/N434S
SEQ ID NO: 1061
NIVMTQTPLSLSVTPGQPASISCKASENVGTYVSWYLKKPGQSPQLLIYGTSNRYTGVPDRFTGSGSATDFTLKISRVEAEDVGVYH
CGQSYRYPYTFGGGTKLEIK/RTVAAPSVFIFPPSKKQLKSGTASVVCLLNNFYPREAKVQWKVDNALQSGNSQESVTEQDSKDST
YSLSSTLTLSKADYEKHKVYACEVTHQGLSSPVTKSFNRGEC
Chain 3 - 069[TL1a]_L1_Q38K/D122K/E123K Light Chain
SEQ ID NO: 1062
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 4 - IL23-A[IL23]_L1_Q38E/F118A Light Chain
SEQ ID NO: 1063
DIQMTQSPSSLSASVGDRVTITCKASRDVAIAVAWYQEKPGKVPKLLIYWASTRHTGVPSRFSGSGSRTDFTLTISSLQPEDVADYF
CHQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP49751 - 111[TL1a]_H1_Q39E/K213E/K218D_L1_Q38K/D122K/E123K_Fab-IL23-
A[IL23]_H1_Q39K/A141F_L1_Q38E/F118A Fab-IgG1_pI(-)_Isosteric_A_PVA_/
S267K/L368D/K370S/M428L/N434S-
IgG1_PVA_/S267K/S364K/E357Q/M428L/N434S
Chain - 111[TL1a]_H1_Q39E/K213E/K218D_IgG1_pI(-)_Isosteric_A_PVA_/
S267K/L368D/K370S/M428L/N434S
SEQ ID NO: 1064
EVQLVESGGGLVQPGGSLRLSCAASGFTFSSFGMHWVREAPGKGLEWVSYISSGSTTIYYADSVKGRFTISRDNAKNSLYLQMNSL
RAEDTAVYYCARAPLLRGAMDYWGQGTLVTVSS/ASTKGPSVFPLAPSSKSTSGGTAALGCLVKDYFPEPVTVSWNSGALTSGVH
TFPAVLQSSGLYSLSSVVTVPSSSLGTQTYICNVNHKPSDTKVDEKVEPDSCDKTHTCPPCPAPPVAGPSVFLFPPKPKDTLMISRTP
EVTCVVVDVKHEDPEVKFNWYVDGVEVHNAKTKPREEEYNSTYRVVSVLTVLHQDWLNGKEYKCKVSNKALPAPIEKTISKAK
GQPREPQVYTLPPSREEMTKNQVSLTCDVSGFYPSDIAVEWESDGQPENNYKTTPPVLDSDGSFFLYSKLTVDKSRWEQGDVFSCS
VLHEALHSHYTQKSLSLSPGK
Chain 2 - IL23-A[IL23]_H1_Q39K/A141F_IgG1_PVA_/S267K/S364K/E357Q/M428L/N434S
SEQ ID NO: 1065
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - 111[TL1a]_L1_Q38K/D122K/E123K Light Chain
SEQ ID NO: 1066
DIVMTQSPDSLAVSLGERATINCKASQDVSTAVVWYQKKPGQSPKLLIYWASTRHTGVPDRFTGSGSGTDYTLTISSLQAEDVAVY
HCQQHYSTPYTFGGGTKLEIK/RTVAAPSVFIFPPSKKQLKSGTASVVCLLNNFYPREAKVQWKVDNALQSGNSQESVTEQDSKDS
TYSLSSTLTLSKADYEKHKVYACEVTHQGLSSPVTKSFNRGEC
Chain 4 - IL23-A[IL23]_L1_Q38E/F118A Light Chain
SEQ ID NO: 1067
DIQMTQSPSSLSASVGDRVTITCKASRDVAIAVAWYQEKPGKVPKLLIYWASTRHTGVPSRFSGSGSRTDFTLTISSLQPEDVADYF
CHQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP50283 - IL23-A[IL23]_H1_Q39E/K213E/K218D_L1_Q38K/D122K/E123K Fab-
041[TL1a]_H1_Q39K/A141F_L1_Q38E/F118A_Fab-IgG1_pI(-) Isosteric_A_L234A/
L235A/P329A/L368D/K370S/M428L/N434S-
IgG1_L234A/L235A/P329A/S364K/E357Q/M428L/N434S
Chain - IL23-A[IL23]_H1_Q39E/K213E/K218D_IgG1_pI(-)_Isosteric_A_L234A/
L235A/P329A/L368D/K370S/M428L/N434S-
IgG1_L234A/L235A/P329A/S364K/E357Q/M428L/N434S
SEQ ID NO: 1068
QVQLVQSGAEVKKPGSSVKVSCKASGYTFTDQTIHWMREAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSDTKVDEKVEPDSCDKTHTCPPCPAPEAAGGPSVFLFPPKPKDTLMISR
TPEVTCVVVDVSHEDPEVKFNWYVDGVEVHNAKTKPREEEYNSTYRVVSVLTVLHQDWLNGKEYKCKVSNKALAAPIEKTISKA
KGQPREPQVYTLPPSREEMTKNQVSLTCDVSGFYPSDIAVEWESDGQPENNYKTTPPVLDSDGSFFLYSKLTVDKSRWEQGDVFSC
SVLHEALHSHYTQKSLSLSPGK
Chain 2 - 041[TL1a]_H1_Q39K/A141F_IgG1_L234A/L235A/P329A/S364K/E357Q/M428L/N434S
SEQ ID NO: 1069
QVQLVQSGAEVKKPGASVKVSCKVSGFSVTSYNVHWVRKAPGQGLEWMGVMWSGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAFLGCLVKDYFPEPVTVSW
NSGALTSGVHTFPAVLQSSGLYSLSSVVTVPSSSLGTQTYICNVNHKPSNTKVDKKVEPKSCDKTHTCPPCPAPEAAGGPSVFLFPP
KPKDTLMISRTPEVTCVVVDVSHEDPEVKFNWYVDGVEVHNAKTKPREEQYNSTYRVVSVLTVLHQDWLNGKEYKCKVSNKAL
AAPIEKTISKAKGQPREPQVYTLPPSREQMTKNQVKLTCLVKGFYPSDIAVEWESNGQPENNYKTTPPVLDSDGSFFLYSKLTVDK
SRWQQGNVFSCSVLHEALHSHYTQKSLSLSPGK
Chain 3 - IL23-A[IL23]_L1_Q38K/D122K/E123K Light Chain
SEQ ID NO: 1070
DIQMTQSPSSLSASVGDRVTITCKASRDVAIAVAWYQKKPGKVPKLLIYWASTRHTGVPSRFSGSGSRTDFTLTISSLQPEDVADYF
CHQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - 041[TL1a]_L1_Q38E/F118A Light Chain
SEQ ID NO: 1071
DIQMTQSPSSVSASVGDRVTITCRASEDIYSGLAWYQEKPGKSPKLLIYGASSLQDGVPSRFSGSGSGTDYTLTISSLQPEDFATYFC
QEGLKSPYTFGAGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP51726 - 041[TL1a]_H1.3_Q39E/K213E/K218D_L1_Q38K/D122K/E123K_Fab-IL23-
A[IL23]_H1_Q39K/A141F_L1_Q38E/F118A Fab-IgG1_pI(-)_Isosteric_A_PVA_/
S267K/L368D/K370S/M428L/N434S-
IgG1_PVA_/S267K/S364K/E357Q/M428L/N434S
Chain - 041[TL1a]_H1.3_Q39E/K213E/K218D_IgG1_pI(-)_Isosteric_A_PVA_/
S267K/L368D/K370S/M428L/N434S SEQ ID NO: 1072
QVQLVQSGAEVKKPGASVKVSCKVSGFSVTSYNVHWVREAPGQGLEWMGVMWSGGNTDYNDKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1_Q39K/A141F_IgG1_PVA_/S267K/S364K/E357Q/M428L/N434S
SEQ ID NO: 1073
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - 041[TL1a]_L1_Q38K/D122K/E123K Light Chain
SEQ ID NO: 1074
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGAGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_L1_Q38E/F118A Light Chain
SEQ ID NO: 1075
DIQMTQSPSSLSASVGDRVTITCKASRDVAIAVAWYQEKPGKVPKLLIYWASTRHTGVPSRFSGSGSRTDFTLTISSLQPEDVADYF
CHQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP51727 - IL23-A[IL23]_H1_Q39E/K213E/K218D_L1_Q38K/D122K/E123K_Fab-
041[TL1a]_H1.3_Q39K/A141F_L1_Q38E/F118A_Fab-IgG1_pI(-)_Isosteric_A_PVA_/
S267K/L368D/K370S/M428L/N434S-
IgG1_PVA_/S267K/S364K/E357Q/M428L/N434S
Chain - IL23-A[IL23]_H1_Q39E/K213E/K218D_IgG1_pI(-)_Isosteric_A_PVA_/
S267K/L368D/K370S/M428L/N434S SEQ ID NO: 1076
QVQLVQSGAEVKKPGSSVKVSCKASGYTFTDQTIHWMREAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSDTKVDEKVEPDSCDKTHTCPPCPAPPVAGPSVFLFPPKPKDTLMISRT
PEVTCVVVDVKHEDPEVKFNWYVDGVEVHNAKTKPREEEYNSTYRVVSVLTVLHQDWLNGKEYKCKVSNKALPAPIEKTISKA
KGQPREPQVYTLPPSREEMTKNQVSLTCDVSGFYPSDIAVEWESDGQPENNYKTTPPVLDSDGSFFLYSKLTVDKSRWEQGDVFSC
SVLHEALHSHYTQKSLSLSPGK
Chain 2 - 041[TL1a]_H1.3_Q39K/A141F_IgG1_PVA_/S267K/S364K/E357Q/M428L/N434S
SEQ ID NO: 1077
QVQLVQSGAEVKKPGASVKVSCKVSGFSVTSYNVHWVRKAPGQGLEWMGVMWSGGNTDYNDKFQGRVTMTRDTSTNTVYME
LSSLRSEDTAVYYCARAGRYYHDSNYYWDFPYWGQGTLVTVSS/ASTKGPSVFPLAPSSKSTSGGTAFLGCLVKDYFPEPVTVSW
NSGALTSGVHTFPAVLQSSGLYSLSSVVTVPSSSLGTQTYICNVNHKPSNTKVDKKVEPKSCDKTHTCPPCPAPPVAGPSVFLFPPK
PKDTLMISRTPEVTCVVVDVKHEDPEVKFNWYVDGVEVHNAKTKPREEQYNSTYRVVSVLTVLHQDWLNGKEYKCKVSNKALP
APIEKTISKAKGQPREPQVYTLPPSREQMTKNQVKLTCLVKGFYPSDIAVEWESNGQPENNYKTTPPVLDSDGSFFLYSKLTVDKSR
WQQGNVFSCSVLHEALHSHYTQKSLSLSPGK
Chain 3 - IL23-A[IL23]_L1_Q38K/D122K/E123K Light Chain
SEQ ID NO: 1078
DIQMTQSPSSLSASVGDRVTITCKASRDVAIAVAWYQKKPGKVPKLLIYWASTRHTGVPSRFSGSGSRTDFTLTISSLQPEDVADYF
CHQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - 041[TL1a]_H1.3_Q39K/A141F_L1_Q38E/F118A Light Chain
SEQ ID NO: 1079
DIQMTQSPSSVSASVGDRVTITCRASEDIYSGLAWYQEKPGKSPKLLIYGASSLQDGVPSRFSGSGSGTDYTLTISSLQPEDFATYFC
QEGLKSPYTFGAGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53089 - 041[TL1a]_H1_Q39E/K213E/K218D_L1_Q38K/D122K/E123K_Fab-IL23-
A[IL23]_H1_Q39K/A141F_L1_Q38E/F118A Fab-IgG1_pI(-)_Isosteric_A_PVA_/M252Y/
S254T/T256E/S267K/L368D/K370S-
IgG1_PVA_/M252Y/S254T/T256E/S267K/S364K/E357Q
Chain - 041[TL1a]_H1_Q39E/K213E/K218D_IgG1_pI(-)_Isosteric_A_PVA_/M252Y/
S254T/T256E/S267K/L368D/K370S SEQ ID NO: 1080
QVQLVQSGAEVKKPGASVKVSCKVSGFSVTSYNVHWVREAPGQGLEWMGVMWSGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLYITREPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIA VEWESDGQPENNYKTTPPVLDSDGSFFLYSKLTVDKSRW
EQGDVFSCSVMHEALHNHYTQKSLSLSPGK
Chain 2 - IL23-A[IL23]_H1_Q39K/A141F_IgG1_PVA_/M252Y/S254T/T256E/S267K/S364K/E357Q
SEQ ID NO: 1081
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLYITRE
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVMHEALHNHYTQKSLSLSPGK
Chain 3 - 041[TL1a]_L1_Q38K/D122K/E123K Light Chain
SEQ ID NO: 1082
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGAGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_L1_Q38E/F118A Light Chain
SEQ ID NO: 1083
DIQMTQSPSSLSASVGDRVTITCKASRDVAIAVAWYQEKPGKVPKLLIYWASTRHTGVPSRFSGSGSRTDFTLTISSLQPEDVADYF
CHQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53369 - 041[TL1a]_H1.324_Q39E/K213E/K218D_L1.27_Q38K/D122K/E123K Fab-IL23-
A[IL23]_H1_Q39K/A141F_L1.117_Q38E/F118A Fab-IgG1_pI(-)_Isosteric_A_PVA_/
S267K/L368D/K370S/M428L/N434S-
IgG1_PVA_/S267K/S364K/E357Q/M428L/N434S
Chain - 041[TL1a]_H1.324_Q39E/K213E/K218D_IgG1_pI(-)_Isosteric_A_PVA_/
S267K/L368D/K370S/M428L/N434S SEQ ID NO: 1084
QVQLVQSGAEVKKPGASVKVSCKVSGFPVTSYNVHWVREAPGQGLEWMGVMWYGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1_Q39K/A141F_IgG1_PVA_/S267K/S364K/E357Q/M428L/N434S
SEQ ID NO: 1085
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - 041[TL1a]_L1.27_Q38K/D122K/E123K Light Chain
SEQ ID NO: 1086
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_L1.117_Q38E/F118A Light Chain
SEQ ID NO: 1087
DIQMTQSPSSLSASVGDRVTITCKASRDVAIAVAWYQEKPGKVPKLLIYWASVRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53370 - 041[TL1a]_H1.324_Q39E/K213E/K218D_L1.27_Q38K/D122K/E123K Fab-IL23-
A[IL23]_H1.187_Q39K/A141F_L1.116_Q38E/F118A Fab-IgG1_pI(-)_Isosteric_A_PVA_/
S267K/L368D/K370S/M428L/N434S-
IgG1_PVA_/S267K/S364K/E357Q/M428L/N434S
Chain - 041[TL1a]_H1.324_Q39E/K213E/K218D_IgG1_pI(-)_Isosteric_A_PVA_/
S267K/L368D/K370S/M428L/N434S SEQ ID NO: 1088
QVQLVQSGAEVKKPGASVKVSCKVSGFPVTSYNVHWVREAPGQGLEWMGVMWYGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1.187_Q39K/A141F_IgG1_PVA_/S267K/S364K/E357Q/M428L/N434S
SEQ ID NO: 1089
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAFFIYWGQGTLVTVSS/ASTKGPSVFPLAPSSKSTSGGTAFLGCLVKDYFPEPVTVSWNSGALTSGVH
TFPAVLQSSGLYSLSSVVTVPSSSLGTQTYICNVNHKPSNTKVDKKVEPKSCDKTHTCPPCPAPPVAGPSVFLFPPKPKDTLMISRTP
EVTCVVVDVKHEDPEVKFNWYVDGVEVHNAKTKPREEQYNSTYRVVSVLTVLHQDWLNGKEYKCKVSNKALPAPIEKTISKAK
GQPREPQVYTLPPSREQMTKNQVKLTCLVKGFYPSDIAVEWESNGQPENNYKTTPPVLDSDGSFFLYSKLTVDKSRWQQGNVFSC
SVLHEALHSHYTQKSLSLSPGK
Chain 3 - 041[TL1a]_L1.27_Q38K/D122K/E123K Light Chain
SEQ ID NO: 1090
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_H1.187_Q39K/A141F_L1.116_Q38E/F118A Light Chain
SEQ ID NO: 1091
DIQMTQSPSSLSASVGDRVTITCKASRDVAIAVAWYQEKPGKVPKLLIYWASQRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53371 - 041[TL1a]_H1.324_Q39E/K213E/K218D_L1.27_Q38K/D122K/E123K Fab-IL23-
A[IL23]_H1.41_Q39K/A141F_L1.117_Q38E/F118A Fab-IgG1_pI(-)_Isosteric_A_PVA_/
S267K/L368D/K370S/M428L/N434S-
IgG1_PVA_/S267K/S364K/E357Q/M428L/N434S
Chain - 041[TL1a]_H1.324_Q39E/K213E/K218D_IgG1_pI(-)_Isosteric_A_PVA_/
S267K/L368D/K370S/M428L/N434S SEQ ID NO: 1092
QVQLVQSGAEVKKPGASVKVSCKVSGFPVTSYNVHWVREAPGQGLEWMGVMWYGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1.41_Q39K/A141F_IgG1_PVA_/S267K/S364K/E357Q/M428L/N434S
SEQ ID NO: 1093
QVQLVQSGAEVKKPGSSVKVSCKASGYTFTDQTLHWMRKAPGQGLEWIGYIYPRDDSPKYNENFKGKVTITADKSTSTAYMELS
SLRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - 041[TL1a]_L1.27_Q38K/D122K/E123K Light Chain
SEQ ID NO: 1094
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_H1.41_Q39K/A141F_L1.117_Q38E/F118A Light Chain
SEQ ID NO: 1095
DIQMTQSPSSLSASVGDRVTITCKASRDVAIAVAWYQEKPGKVPKLLIYWASVRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53372 - 041[TL1a]_H1.318_Q39E/K213E/K218D_L1.27_Q38K/D122K/E123K Fab-IL23-
A[IL23]_H1_Q39K/A141F_L1.117_Q38E/F118A Fab-IgG1_pI(-)_Isosteric_A_PVA_/
S267K/L368D/K370S/M428L/N434S-
IgG1_PVA_/S267K/S364K/E3570/M428L/N434S
Chain - 041[TL1a]_H1.318_Q39E/K213E/K218D_IgG1_pI(-)_Isosteric_A_PVA_/
S267K/L368D/K370S/M428L/N434S SEQ ID NO: 1096
QVQLVQSGAEVKKPGASVKVSCKVSGFSVTSYNVHWVREAPGQGLEWMGVMWFGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIA VEWESDGQPENNYKTTPPVLDSDGSFFLYSKLTVDKSRW
EQGDVFSCSVLHEALHSHYTQKSLSLSPGK
Chain 2 - IL23-A[IL23]_H1_Q39K/A141F_IgG1_PVA_/S267K/S364K/E357Q/M428L/N434S
SEQ ID NO: 1097
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - 041[TL1a]_L1.27_Q38K/D122K/E123K Light Chain
SEQ ID NO: 1098
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_L1.117_Q38E/F118A Light Chain
SEQ ID NO: 1099
DIQMTQSPSSLSASVGDRVTITCKASRDVAIAVAWYQEKPGKVPKLLIYWASVRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53373 - 041[TL1a]_H1.318_Q39E/K213E/K218D_L1.27_Q38K/D122K/E123K_Fab-IL23-
A[IL23]_H1.187_Q39K/A141F_L1.116_Q38E/F118A Fab-IgG1_pI(-)_Isosteric_A_PVA_/
S267K/L368D/K370S/M428L/N434S-
IgG1_PVA_/S267K/S364K/E357Q/M428L/N434S
Chain - 041[TL1a]_H1.318_Q39E/K213E/K218D_IgG1_pI(-)_Isosteric_A_PVA_/
S267K/L368D/K370S/M428L/N434S SEQ ID NO: 1100
QVQLVQSGAEVKKPGASVKVSCKVSGFSVTSYNVHWVREAPGQGLEWMGVMWFGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1.187_Q39K/A141F_IgG1_PVA_/S267K/S364K/E357Q/M428L/N434S
SEQ ID NO: 1101
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAFFIYWGQGTLVTVSS/ASTKGPSVFPLAPSSKSTSGGTAFLGCLVKDYFPEPVTVSWNSGALTSGVH
TFPAVLQSSGLYSLSSVVTVPSSSLGTQTYICNVNHKPSNTKVDKKVEPKSCDKTHTCPPCPAPPVAGPSVFLFPPKPKDTLMISRTP
EVTCVVVDVKHEDPEVKFNWYVDGVEVHNAKTKPREEQYNSTYRVVSVLTVLHQDWLNGKEYKCKVSNKALPAPIEKTISKAK
GQPREPQVYTLPPSREQMTKNQVKLTCLVKGFYPSDIAVEWESNGQPENNYKTTPPVLDSDGSFFLYSKLTVDKSRWQQGNVFSC
SVLHEALHSHYTQKSLSLSPGK
Chain 3 - 041[TL1a]_L1.27_Q38K/D122K/E123K Light Chain
SEQ ID NO: 1102
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_H1.187_Q39K/A141F_L1.116_Q38E/F118A Light Chain
SEQ ID NO: 1103
DIQMTQSPSSLSASVGDRVTITCKASRDVAIAVAWYQEKPGKVPKLLIYWASQRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53374 - 041[TL1a]_H1.318_Q39E/K213E/K218D_L1.27_Q38K/D122K/E123K Fab-IL23-
A[IL23]_H1.41_Q39K/A141F_L1.117_Q38E/F118A Fab-IgG1_pI(-)_Isosteric_A_PVA_/
S267K/L368D/K370S/M428L/N434S-
IgG1_PVA_/S267K/S364K/E357Q/M428L/N434S
Chain - 041[TL1a]_H1.318_Q39E/K213E/K218D_IgG1_pI(-)_Isosteric_A_PVA_/
S267K/L368D/K370S/M428L/N434S SEQ ID NO: 1104
QVQLVQSGAEVKKPGASVKVSCKVSGFSVTSYNVHWVREAPGQGLEWMGVMWFGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1.41_Q39K/A141F_IgG1_PVA_/S267K/S364K/E357Q/M428L/N434S
SEQ ID NO: 1105
QVQLVQSGAEVKKPGSSVKVSCKASGYTFTDQTLHWMRKAPGQGLEWIGYIYPRDDSPKYNENFKGKVTITADKSTSTAYMELS
SLRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - 041[TL1a]_L1.27_Q38K/D122K/E123K Light Chain
SEQ ID NO: 1106
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_H1.41_Q39K/A141F_L1.117_Q38E/F118A Light Chain
SEQ ID NO: 1107
DIQMTQSPSSLSASVGDRVTITCKASRDVAIAVAWYQEKPGKVPKLLIYWASVRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53375 - 041[TL1a]_H1.447_Q39E/K213E/K218D_L1.27_Q38K/D122K/E123K_Fab-IL23-
A[IL23]_H1_Q39K/A141F_L1.117_Q38E/F118A_Fab-IgG1_pI(-)_Isosteric_A_PVA_/
S267K/L368D/K370S/M428L/N434S-
IgG1_PVA_/S267K/S364K/E357Q/M428L/N434S
Chain - 041[TL1a]_H1.447_Q39E/K213E/K218D_IgG1_pI(-)_Isosteric_A_PVA_/
S267K/L368D/K370S/M428L/N434S SEQ ID NO: 1108
QVQLVQSGAEVKKPGASVKVSCKVSGFSVTRYNVHWVREAPGQGLEWMGVMWSGANTDYNSKFQGRVTMTRDTSTNTVYME
LSSLRSEDTAVYYCARAGRYYVDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1_Q39K/A141F_IgG1_PVA_/S267K/S364K/E357Q/M428L/N434S
SEQ ID NO: 1109
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - 041[TL1a]_L1.27_Q38K/D122K/E123K Light Chain
SEQ ID NO: 1110
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_L1.117_Q38E/F118A Light Chain
SEQ ID NO: 1111
DIQMTQSPSSLSASVGDRVTITCKASRDVAIAVAWYQEKPGKVPKLLIYWASVRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53376 - 041[TL1a]_H1.447_Q39E/K213E/K218D_L1.27_Q38K/D122K/E123K Fab-IL23-
A[IL23]_H1.187_Q39K/A141F_L1.116_Q38E/F118A Fab-IgG1_pI(-)_Isosteric_A_PVA_/
S267K/L368D/K370S/M428L/N434S-
IgG1_PVA_/S267K/S364K/E357Q/M428L/N434S
Chain - 041[TL1a]_H1.447_Q39E/K213E/K218D_IgG1_pI(-)_Isosteric_A_PVA_/
S267K/L368D/K370S/M428L/N434S SEQ ID NO: 1112
QVQLVQSGAEVKKPGASVKVSCKVSGFSVTRYNVHWVREAPGQGLEWMGVMWSGANTDYNSKFQGRVTMTRDTSTNTVYME
LSSLRSEDTAVYYCARAGRYYVDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1.187_Q39K/A141F_IgG1_PVA_/S267K/S364K/E357Q/M428L/N434S
SEQ ID NO: 1113
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAFFIYWGQGTLVTVSS/ASTKGPSVFPLAPSSKSTSGGTAFLGCLVKDYFPEPVTVSWNSGALTSGVH
TFPAVLQSSGLYSLSSVVTVPSSSLGTQTYICNVNHKPSNTKVDKKVEPKSCDKTHTCPPCPAPPVAGPSVFLFPPKPKDTLMISRTP
EVTCVVVDVKHEDPEVKFNWYVDGVEVHNAKTKPREEQYNSTYRVVSVLTVLHQDWLNGKEYKCKVSNKALPAPIEKTISKAK
GQPREPQVYTLPPSREQMTKNQVKLTCLVKGFYPSDIAVEWESNGQPENNYKTTPPVLDSDGSFFLYSKLTVDKSRWQQGNVFSC
SVLHEALHSHYTQKSLSLSPGK
Chain 3 - 041[TL1a]_L1.27_Q38K/D122K/E123K Light Chain
SEQ ID NO: 1114
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_H1.187_Q39K/A141F_L1.116_Q38E/F118A Light Chain
SEQ ID NO: 1115
DIQMTQSPSSLSASVGDRVTITCKASRDVAIAVAWYQEKPGKVPKLLIYWASQRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53377 - 041[TL1a]_H1.447_Q39E/K213E/K218D_L1.27_Q38K/D122K/E123K Fab-IL23-
A[IL23]_H1.41_Q39K/A141F_L1.117_Q38E/F118A_Fab-IgG1_pI(-)_Isosteric_A_PVA_/
S267K/L368D/K370S/M428L/N434S-
IgG1_PVA_/S267K/S364K/E357Q/M428L/N434S
Chain - 041[TL1a]_H1.447_Q39E/K213E/K218D_IgG1_pI(-)_Isosteric_A_PVA_/
S267K/L368D/K370S/M428L/N434S SEQ ID NO: 1116
QVQLVQSGAEVKKPGASVKVSCKVSGFSVTRYNVHWVREAPGQGLEWMGVMWSGANTDYNSKFQGRVTMTRDTSTNTVYME
LSSLRSEDTAVYYCARAGRYYVDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1.41_Q39K/A141F_IgG1_PVA_/S267K/S364K/E357Q/M428L/N434S
SEQ ID NO: 1117
QVQLVQSGAEVKKPGSSVKVSCKASGYTFTDQTLHWMRKAPGQGLEWIGYIYPRDDSPKYNENFKGKVTITADKSTSTAYMELS
SLRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - 041[TL1a]_L1.27_Q38K/D122K/E123K Light Chain
SEQ ID NO: 1118
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_H1.41_Q39K/A141F_L1.117_Q38E/F118A Light Chain
SEQ ID NO: 1119
DIQMTQSPSSLSASVGDRVTITCKASRDVAIAVAWYQEKPGKVPKLLIYWASVRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53378 - 041[TL1a]_H1.239_Q39E/K213E/K218D_L1.27_Q38K/D122K/E123K Fab-IL23-
A[IL23]_H1_Q39K/A141F_L1.117_Q38E/F118A Fab-IgG1_pI(-)_Isosteric_A_PVA_/
S267K/L368D/K370S/M428L/N434S-
IgG1_PVA_/S267K/S364K/E357Q/M428L/N434S
Chain - 041[TL1a]_H1.239_Q39E/K213E/K218D_IgG1_pI(-)_Isosteric_A_PVA_/
S267K/L368D/K370S/M428L/N434S SEQ ID NO: 1120
QVQLVQSGAEVKKPGASVKVSCKVSGFSVTSYNVHWVREAPGQGLEWMGVMWRGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1_Q39K/A141F_IgG1_PVA_/S267K/S364K/E357Q/M428L/N434S
SEQ ID NO: 1121
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - 041[TL1a]_L1.27_Q38K/D122K/E123K Light Chain
SEQ ID NO: 1122
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_L1.117_Q38E/F118A Light Chain
SEQ ID NO: 1123
DIQMTQSPSSLSASVGDRVTITCKASRDVAIAVAWYQEKPGKVPKLLIYWASVRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53379 - 041[TL1a]_H1.239_Q39E/K213E/K218D_L1.27_Q38K/D122K/E123K Fab-IL23-
A[IL23]_H1.187_Q39K/A141F_L1.116_Q38E/F118A_Fab-IgG1_pI(-)_Isosteric_A_PVA_/
S267K/L368D/K370S/M428L/N434S-
IgG1_PVA_/S267K/S364K/E357Q/M428L/N434S
Chain - 041[TL1a]_H1.239_Q39E/K213E/K218D_IgG1_pI(-)_Isosteric_A_PVA_/
S267K/L368D/K370S/M428L/N434S SEQ ID NO: 1124
QVQLVQSGAEVKKPGASVKVSCKVSGFSVTSYNVHWVREAPGQGLEWMGVMWRGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1.187_Q39K/A141F_IgG1_PVA_/S267K/S364K/E357Q/M428L/N434S
SEQ ID NO: 1125
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAFFIYWGQGTLVTVSS/ASTKGPSVFPLAPSSKSTSGGTAFLGCLVKDYFPEPVTVSWNSGALTSGVH
TFPAVLQSSGLYSLSSVVTVPSSSLGTQTYICNVNHKPSNTKVDKKVEPKSCDKTHTCPPCPAPPVAGPSVFLFPPKPKDTLMISRTP
EVTCVVVDVKHEDPEVKFNWYVDGVEVHNAKTKPREEQYNSTYRVVSVLTVLHQDWLNGKEYKCKVSNKALPAPIEKTISKAK
GQPREPQVYTLPPSREQMTKNQVKLTCLVKGFYPSDIAVEWESNGQPENNYKTTPPVLDSDGSFFLYSKLTVDKSRWQQGNVFSC
SVLHEALHSHYTQKSLSLSPGK
Chain 3 - 041[TL1a]_L1.27_Q38K/D122K/E123K Light Chain
SEQ ID NO: 1126
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_H1.187_Q39K/A141F_L1.116_Q38E/F118A Light Chain
SEQ ID NO: 1127
DIQMTQSPSSLSASVGDRVTITCKASRDVAIAVAWYQEKPGKVPKLLIYWASQRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53380 - 041[TL1a]_H1.239_Q39E/K213E/K218D_L1.27_Q38K/D122K/E123K_Fab-IL23-
A[IL23]_H1.41_Q39K/A141F_L1.117_Q38E/F118A Fab-IgG1 pI(-)_Isosteric_A_PVA_/
S267K/L368D/K370S/M428L/N434S-
IgG1_PVA_/S267K/S364K/E357Q/M428L/N434S
Chain - 041[TL1a]_H1.239_Q39E/K213E/K218D_IgG1_pI(-)_Isosteric_A_PVA_/
S267K/L368D/K370S/M428L/N434S SEQ ID NO: 1128
QVQLVQSGAEVKKPGASVKVSCKVSGFSVTSYNVHWVREAPGQGLEWMGVMWRGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1.41_Q39K/A141F_IgG1_PVA_/S267K/S364K/E357Q/M428L/N434S
SEQ ID NO: 1129
QVQLVQSGAEVKKPGSSVKVSCKASGYTFTDQTLHWMRKAPGQGLEWIGYIYPRDDSPKYNENFKGKVTITADKSTSTAYMELS
SLRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - 041[TL1a]_L1.27_Q38K/D122K/E123K Light Chain
SEQ ID NO: 1130
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_H1.41_Q39K/A141F_L1.117_Q38E/F118A Light Chain
SEQ ID NO: 1131
DIQMTQSPSSLSASVGDRVTITCKASRDVAIAVAWYQEKPGKVPKLLIYWASVRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53412 - 041[TL1a]_H1.337_Q39E/K213E/K218D_L1.27_Q38K/D122K/E123K_Fab-IL23-
A[IL23]_H1_Q39K/A141F_L1.117_Q38E/F118A_Fab-IgG1_pI(-) Isosteric_A_PVA_/
S267K/L368D/K370S/M428L/N434S-
IgG1_PVA_/S267K/S364K/E357Q/M428L/N434S
Chain - 041[TL1a]_H1.337_Q39E/K213E/K218D_IgG1_pI(-)_Isosteric_A_PVA_/
S267K/L368D/K370S/M428L/N434S SEQ ID NO: 1132
QVQLVQSGAEVKKPGASVKVSCKVSGFSVTSYNVHWVREAPGQGLEWMGVMWYGGNTDYNSKFQGRVTMTRDTSTNTVYME
LSSLRSEDTAVYYCARAGRYYVDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1_Q39K/A141F_IgG1_PVA_/S267K/S364K/E357Q/M428L/N434S
SEQ ID NO: 1133
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - 041[TL1a]_L1.27_Q38K/D122K/E123K Light Chain
SEQ ID NO: 1134
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_L1.117_Q38E/F118A Light Chain
SEQ ID NO: 1135
DIQMTQSPSSLSASVGDRVTITCKASRDVAIAVAWYQEKPGKVPKLLIYWASVRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53413 - 041[TL1a]_H1.337_Q39E/K213E/K218D_L1.27_Q38K/D122K/E123K_Fab-IL23-
A[IL23]_H1.187_Q39K/A141F_L1.116_Q38E/F118A_Fab-IgG1_pI(-)_Isosteric_A_PVA_/
S267K/L368D/K370S/M428L/N434S-
IgG1_PVA_/S267K/S364K/E357Q/M428L/N434S
Chain - 041[TL1a]_H1.337_Q39E/K213E/K218D_IgG1_pI(-)_Isosteric_A_PVA_/
S267K/L368D/K370S/M428L/N434S SEQ ID NO: 1136
QVQLVQSGAEVKKPGASVKVSCKVSGFSVTSYNVHWVREAPGQGLEWMGVMWYGGNTDYNSKFQGRVTMTRDTSTNTVYME
LSSLRSEDTAVYYCARAGRYYVDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1.187_Q39K/A141F_IgG1_PVA_/S267K/S364K/E357Q/M428L/N434S
SEQ ID NO: 1137
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAFFIYWGQGTLVTVSS/ASTKGPSVFPLAPSSKSTSGGTAFLGCLVKDYFPEPVTVSWNSGALTSGVH
TFPAVLQSSGLYSLSSVVTVPSSSLGTQTYICNVNHKPSNTKVDKKVEPKSCDKTHTCPPCPAPPVAGPSVFLFPPKPKDTLMISRTP
EVTCVVVDVKHEDPEVKFNWYVDGVEVHNAKTKPREEQYNSTYRVVSVLTVLHQDWLNGKEYKCKVSNKALPAPIEKTISKAK
GQPREPQVYTLPPSREQMTKNQVKLTCLVKGFYPSDIAVEWESNGQPENNYKTTPPVLDSDGSFFLYSKLTVDKSRWQQGNVFSC
SVLHEALHSHYTQKSLSLSPGK
Chain 3 - 041[TL1a]_L1.27_Q38K/D122K/E123K Light Chain
SEQ ID NO: 1138
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_H1.187_Q39K/A141F_L1.116_Q38E/F118A Light Chain
SEQ ID NO: 1139
DIQMTQSPSSLSASVGDRVTITCKASRDVAIAVAWYQEKPGKVPKLLIYWASQRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53414 - 041[TL1a]_H1.337_Q39E/K213E/K218D_L1.27_Q38K/D122K/E123K Fab-IL23-
A[IL23]_H1.41_Q39K/A141F_L1.117_Q38E/F118A Fab-IgG1_pI(-)_Isosteric_A_PVA_/
S267K/L368D/K370S/M428L/N434S-
IgG1_PVA_/S267K/S364K/E357Q/M428L/N434S
Chain - 041[TL1a]_H1.337_Q39E/K213E/K218D_IgG1_pI(-)_Isosteric_A_PVA_/
S267K/L368D/K370S/M428L/N434S SEQ ID NO: 1140
QVQLVQSGAEVKKPGASVKVSCKVSGFSVTSYNVHWVREAPGQGLEWMGVMWYGGNTDYNSKFQGRVTMTRDTSTNTVYME
LSSLRSEDTAVYYCARAGRYYVDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2 - IL23-A[IL23]_H1.41_Q39K/A141F_IgG1_PVA_/S267K/S364K/E357Q/M428L/N434S
SEQ ID NO: 1141
QVQLVQSGAEVKKPGSSVKVSCKASGYTFTDQTLHWMRKAPGQGLEWIGYIYPRDDSPKYNENFKGKVTITADKSTSTAYMELS
SLRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - 041[TL1a]_L1.27_Q38K/D122K/E123K Light Chain
SEQ ID NO: 1142
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_H1.41_Q39K/A141F_L1.117_Q38E/F118A Light Chain
SEQ ID NO: 1143
DIQMTQSPSSLSASVGDRVTITCKASRDVAIAVAWYQEKPGKVPKLLIYWASVRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP55903 - 111[TL1a]_H1_Q39E/K213E/K218D_L1_Q38K/D122K/E123K_Fab-IL23-
A[IL23]_H1_Q39K/A141F_L1.117_Q38E/F118A Fab-IgG1_pI(-) Isosteric_A_PVA_/
S267K/L368D/K370S/M428L/N434S-
IgG1_PVA_/S267K/S364K/E357Q/M428L/N434S
Chain - 111[TL1a]_H1_Q39E/K213E/K218D_IgG1_pI(-)_Isosteric_A_PVA_/
S267K/L368D/K370S/M428L/N434S
SEQ ID NO: 1144
EVQLVESGGGLVQPGGSLRLSCAASGFTFSSFGMHWVREAPGKGLEWVSYISSGSTTIYYADSVKGRFTISRDNAKNSLYLQMNSL
RAEDTAVYYCARAPLLRGAMDYWGQGTLVTVSS/ASTKGPSVFPLAPSSKSTSGGTAALGCLVKDYFPEPVTVSWNSGALTSGVH
TFPAVLQSSGLYSLSSVVTVPSSSLGTQTYICNVNHKPSDTKVDEKVEPDSCDKTHTCPPCPAPPVAGPSVFLFPPKPKDTLMISRTP
EVTCVVVDVKHEDPEVKFNWYVDGVEVHNAKTKPREEEYNSTYRVVSVLTVLHQDWLNGKEYKCKVSNKALPAPIEKTISKAK
GQPREPQVYTLPPSREEMTKNQVSLTCDVSGFYPSDIAVEWESDGQPENNYKTTPPVLDSDGSFFLYSKLTVDKSRWEQGDVFSCS
VLHEALHSHYTQKSLSLSPGK
Chain 2 - IL23-A[IL23]_H1_Q39K/A141F_IgG1_PVA_/S267K/S364K/E357Q/M428L/N434S
SEQ ID NO: 1145
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3 - 111[TL1a]_L1_Q38K/D122K/E123K Light Chain
SEQ ID NO: 1146
DIVMTQSPDSLAVSLGERATINCKASQDVSTAVVWYQKKPGQSPKLLIYWASTRHTGVPDRFTGSGSGTDYTLTISSLQAEDVAVY
HCQQHYSTPYTFGGGTKLEIK/RTVAAPSVFIFPPSKKQLKSGTASVVCLLNNFYPREAKVQWKVDNALQSGNSQESVTEQDSKDS
TYSLSSTLTLSKADYEKHKVYACEVTHQGLSSPVTKSFNRGEC
Chain 4 - IL23-A[IL23]_L1.117_Q38E/F118A Light Chain
SEQ ID NO: 1147
DIQMTQSPSSLSASVGDRVTITCKASRDVAIAVAWYQEKPGKVPKLLIYWASVRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP55904 - 111[TL1a]_H1_Q39E/K213E/K218D_L1_Q38K/D122K/E123K_Fab-IL23-
A[IL23]_H1.187_Q39K/A141F_L1.116_Q38E/F118A Fab-IgG1 pI(-)_Isosteric_A_PVA_/
S267K/L368D/K370S/M428L/N434S-
IgG1_PVA_/S267K/S364K/E357Q/M428L/N434S
Chain - 111[TL1a]_H1_Q39E/K213E/K218D_IgG1_pI(-)_Isosteric_A_PVA_/S267K/
L368D/K370S/M428L/N434S
SEQ ID NO: 1148
EVQLVESGGGLVQPGGSLRLSCAASGFTFSSFGMHWVREAPGKGLEWVSYISSGSTTIYYADSVKGRFTISRDNAKNSLYLQMNSL
RAEDTAVYYCARAPLLRGAMDYWGQGTLVTVSS/ASTKGPSVFPLAPSSKSTSGGTAALGCLVKDYFPEPVTVSWNSGALTSGVH
TFPAVLQSSGLYSLSSVVTVPSSSLGTQTYICNVNHKPSDTKVDEKVEPDSCDKTHTCPPCPAPPVAGPSVFLFPPKPKDTLMISRTP
EVTCVVVDVKHEDPEVKFNWYVDGVEVHNAKTKPREEEYNSTYRVVSVLTVLHQDWLNGKEYKCKVSNKALPAPIEKTISKAK
GQPREPQVYTLPPSREEMTKNQVSLTCDVSGFYPSDIAVEWESDGQPENNYKTTPPVLDSDGSFFLYSKLTVDKSRWEQGDVFSCS
VLHEALHSHYTQKSLSLSPGK
Chain 2 - IL23-A[IL23]_H1.187_Q39K/A141F_IgG1_PVA_/S267K/S364K/E357Q/M428L/N434S
SEQ ID NO: 1149
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAFFIYWGQGTLVTVSS/ASTKGPSVFPLAPSSKSTSGGTAFLGCLVKDYFPEPVTVSWNSGALTSGVH
TFPAVLQSSGLYSLSSVVTVPSSSLGTQTYICNVNHKPSNTKVDKKVEPKSCDKTHTCPPCPAPPVAGPSVFLFPPKPKDTLMISRTP
EVTCVVVDVKHEDPEVKFNWYVDGVEVHNAKTKPREEQYNSTYRVVSVLTVLHQDWLNGKEYKCKVSNKALPAPIEKTISKAK
GQPREPQVYTLPPSREQMTKNQVKLTCLVKGFYPSDIA VEWESNGQPENNYKTTPPVLDSDGSFFLYSKLTVDKSRWQQGNVFSC
SVLHEALHSHYTQKSLSLSPGK
Chain 3 - 111[TL1a]_L1_Q38K/D122K/E123K Light Chain
SEQ ID NO: 1150
DIVMTQSPDSLAVSLGERATINCKASQDVSTAVVWYQKKPGQSPKLLIYWASTRHTGVPDRFTGSGSGTDYTLTISSLQAEDVAVY
HCQQHYSTPYTFGGGTKLEIK/RTVAAPSVFIFPPSKKQLKSGTASVVCLLNNFYPREAKVQWKVDNALQSGNSQESVTEQDSKDS
TYSLSSTLTLSKADYEKHKVYACEVTHQGLSSPVTKSFNRGEC
Chain 4 - IL23-A[IL23]_H1.187_Q39K/A141F_L1.116_Q38E/F118A Light Chain
SEQ ID NO: 1151
DIQMTQSPSSLSASVGDRVTITCKASRDVAIAVAWYQEKPGKVPKLLIYWASQRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP56215 - 041[TL1a]_H1.337_Q39E/K213E/K218D_L1.27 Q38K/D122K/E123K Fab-IL23-
A[IL23]_H1_Q39K/A141F_L1.117_Q38E/F118A Fab-IgG1 pI(-)_Isosteric_A_PVA_/
S267K/L368D/K370S-IgG1_PVA_/S267K/S364K/E357Q
Chain - 041[TL1a]_H1.337_Q39E/K213E/K218D_IgG1_pI(-)_Isosteric_A_PVA_/
S267K/L368D/K370S
SEQ ID NO: 1152
QVQLVQSGAEVKKPGASVKVSCKVSGFSVTSYNVHWVREAPGQGLEWMGVMWYGGNTDYNSKFQGRVTMTRDTSTNTVYME
LSSLRSEDTAVYYCARAGRYYVDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVMHEALHNHYTQKSLSLSPGK
Chain 2 - IL23-A[IL23]_H1_Q39K/A141F_IgG1_PVA_/S267K/S364K/E357Q
SEQ ID NO: 1153
QVQLVQSGAEVKKPGSSVKVSCKASGYTFTDQTIHWMRKAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAFLGCLVKDYFPEPVTVSWNSGALTSGV
HTFPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVMHEALHNHYTQKSLSLSPGK
Chain 3 - 041[TL1a]_L1.27_Q38K/D122K/E123K Light Chain
SEQ ID NO: 1154
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGQGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4 - IL23-A[IL23]_L1.117_Q38E/F118A Light Chain
SEQ ID NO: 1155
DIQMTQSPSSLSASVGDRVTITCKASRDVAIAVAWYQEKPGKVPKLLIYWASVRHTGVPSRFSGSGSRTDFTLTISSLQPEDVADYF
CYQYSSYPFTFGSGTKLEIK/RTVAAPSVFIAPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC

TABLE 30

| | SEQ ID NO \| Antigen | | | | | | | |
|---|---|---|---|---|---|---|---|---|
| Xencor ID | Chain 1 | | Chain 2 | | Chain 3 | | Chain 4 | |
| XENC001 | 2272 | TL1A | 2273 | IL23 | 2274 | TL1A | 2275 | IL23 |
| XENC002 | 2276 | TL1A | 2277 | IL23 | 2278 | TL1A | 2279 | IL23 |
| XENC003 | 2280 | TL1A | 2281 | IL23 | 2282 | TL1A | 2283 | IL23 |
| XENC004 | 2284 | TL1A | 2285 | IL23 | 2286 | TL1A | 2287 | IL23 |
| XENC005 | 2288 | TL1A | 2289 | IL23 | 2290 | TL1A | 2291 | IL23 |
| XENC006 | 2292 | TL1A | 2293 | IL23 | 2294 | TL1A | 2295 | IL23 |
| XENC007 | 2296 | TL1A | 2297 | IL23 | 2298 | TL1A | 2299 | IL23 |
| XENC008 | 2300 | TL1A | 2301 | IL23 | 2302 | TL1A | 2303 | IL23 |
| XENC009 | 2304 | TL1A | 2305 | IL23 | 2306 | TL1A | 2307 | IL23 |
| XENC010 | 2308 | TL1A | 2309 | IL23 | 2310 | TL1A | 2311 | IL23 |

### 3C: CrossMab 1+1 Format

Another heterodimeric antibody format that finds particular use in subject anti-TL1A×anti-IL23 antibodies provided herein is the “CrossMab 1+1” format (see FIG. 1, Panel Q). The CrossMab 1+1 format is a Fab×Fab four chain bispecific antibody. In some embodiments, the first monomer includes a first variable light domain (VL1) attached to a heavy chain constant domain comprising a CH1-hinge-CH2-CH3, wherein CH2-CH3 is a first Fc domain; and the second monomer includes a first variable heavy domain (VH1) attached to a lambda constant light domain (e.g., a CLκ). In some embodiments, the first monomer includes a first variable heavy domain (VH1) attached to a lambda constant light domain attached to CH2-CH3 via a domain linker; and the second monomer includes a first variable light domain (VL1) attached to a CH1-partial hinge. The third monomer comprises a canonical heavy chain that includes a VH2-CH1-hinge-CH2-CH3, wherein VH2 is a second variable heavy domain and CH2-CH3 is a second Fc domain. The fourth monomer comprises a canonical light chain that includes a VL2-CL, wherein VL2 is a second variable light domain and CL is a constant light domain (e.g., a CLκ). The first monomer and second monomer form a “crossed” arm, wherein VL1 and VH1 form a first antigen binding domain that binds a first antigen, and the third monomer and forth monomer form a “non-crossed” arm, wherein VL2 and VH2 form a second antigen binding domain that binds a second antigen.

### 3C(1): CrossMab-VH-VL Charge-Swap 1+1 Format

In some embodiments, the CH1 of the first monomer and the CL domain (e.g., CLκ) of the fourth monomer further include “charge” swap amino acid substitutions. In some embodiments, these “charge” swap substitutions comprise amino acid substitutions K213E/K218D (EU numbering) in the CH1 of the third monomer, and the fourth monomer CL is a CLκ that comprises amino acid substitutions D112K/E123K (Kabat numbering).

Without being bound by any particular theory of operation, it is believed that the crossing of one arm (i.e., the “crossed” arm) and charge swap engineering on the “non-crossed” arm in this format greatly diminishes the mispairing of LCs without affecting the various other developability and biophysical parameters that might be used in development of a biological therapeutic.

In some embodiments, the constant light domain of the second monomer includes amino acid substitutions R108A/T109S (Kabat numbering). In some embodiments, the VL1 of the first monomer is attached to the CH1 of the first monomer by an SS linker. In some embodiments, the “crossed” arm includes isosteric variants N208D/Q295E/N384D/Q418E/N421D in the heavy chain constant domain of the first monomer, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K.

Exemplary anti-TL1A×anti-IL23 bispecific antibodies in the CrossMab 1+1 format are depicted in Table 31. CDRs are underlined and slashes indicate the border(s) between the variable regions, linkers, Fc regions, and constant domains. It should be noted that the TL1A×IL23 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S or any other half-life extension variants and/or any other ablation variant (as depicted in Table 1) in one or preferably both Fc domains Additionally, each sequence can utilize any other heterodimeric Fc variant as depicted in Tables 12-17.

TABLE 31

| | SEQ ID NO \| Antigen | | | | | | | |
|---|---|---|---|---|---|---|---|---|
| Xencor ID | Chain 1 | | Chain 2 | | Chain 3 | | Chain 4 | |
| XENP51595 | 1176 | TL1A | 1177 | IL23 | 1178 | TL1A | 1179 | IL23 |
| XENP51596 | 1180 | TL1A | 1181 | IL23 | 1182 | TL1A | 1183 | IL23 |
| XENP51597 | 1184 | TL1A | 1185 | IL23 | 1186 | TL1A | 1187 | IL23 |
| XENP51598 | 1188 | IL23 | 1189 | TL1A | 1190 | IL23 | 1191 | TL1A |
| XENP51599 | 1192 | IL23 | 1193 | TL1A | 1194 | IL23 | 1195 | TL1A |
| XENP51600 | 1196 | IL23 | 1197 | TL1A | 1198 | IL23 | 1199 | TL1A |

Exemplary anti-TL1A×anti-IL23 bispecific antibodies in the CrossMab-VH-VL charge-swap 1+1 format are depicted in Table 32. CDRs are underlined and slashes indicate the border(s) between the variable regions, linkers, Fc regions, and constant domains. It should be noted that the TL1A×IL23 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S or any other half-life extension variants and/or any other ablation variant (as depicted in Table 1) in one or preferably both Fc domains Additionally, each sequence can utilize any other heterodimeric Fc variant as depicted in Tables 12-17.

TABLE 32
XENP52515-041[TL1a]_H1_K147E/K213E_L1_E123R/Q124K_Fab-IL23-A[IL23]_L1_CH1_H1_Ckappa_crossMab_
VH_VL-IgG1_pI(-)_Isosteric_A_PVA_/S267K/L368D/K370S/M428L/N434S-IgG1_PVA/S267K/S364K/E357Q/
M428L/N434S
Chain 1-041[TL1a]_H1_K147E/K213E_IgG1_pI(-)_Isosteric_A_PVA_/S267K/L368D/K370S/M428L/
N434S SEQ ID NO: 1291
QVQLVQSGAEVKKPGASVKVSCKVSGFSVTSYNVHWVRQAPGQGLEWMGVMWSGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVEDYFPEPVTVSW
NSGALTSGVHTFPAVLQSSGLYSLSSVVTVPSSSLGTQTYICNVNHKPSDTKVDEKVEPKSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2-IL23-A[IL23]_L1_CH1_IgG1_PVA_/S267K/S364K/E357Q/M428L/N434S SEQ ID NO: 1292
DIQMTQSPSSLSASVGDRVTITCKASRDVAIAVAWYQQKPGKVPKLLIYWASTRHTGVPSRFSGSGSRTDFTLTISSLQPEDVADYF
CHQYSSYPFTFGSGTKLEIKSS/ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPPVAGPSVFLFPPKPKDTLMISRTPEVTCVVVDVKHED
PEVKFNWYVDGVEVHNAKTKPREEQYNSTYRVVSVLTVLHQDWLNGKEYKCKVSNKALPAPIEKTISKAKGQPREPQVYTLPPS
REQMTKNQVKLTCLVKGFYPSDIAVEWESNGQPENNYKTTPPVLDSDGSFFLYSKLTVDKSRWQQGNVFSCSVLHEALHSHYTQ
KSLSLSPGK
Chain 3-041[TL1a]_L1_E123R/Q124K Light Chain SEQ ID NO: 1293
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGAGTKLEIK/RTVAAPSVFIFPPSDRKLKSGTASVVCLLNNFYPREAKVQWKVDNALQSGNSQESVTEQDSKDSTYS
LSSTLTLSKADYEKHKVYACEVTHQGLSSPVTKSFNRGEC
Chain 4-IL23-A[IL23]_H1_Ckappa Light Chain SEQ ID NO: 1294
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAWFIYWGQGTLVTVSS/ASVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
XENP52516-041[TL1a]_H1_K213E/K218D_L1_D122K/E123K_Fab-IL23-A[IL23]_L1_CH1_H1_Ckappa_
crossMab_VH_VL-IgG1_pI(-)_Isosteric_A_PVA_/S267K/L368D/K370S/M428L/N434S-IgG1_PVA_/S267K/
S364K/E357Q/M428L/N434S
Chain 1-041[TL1a]_H1_K213E/K218D_IgG1_pI(-)_Isosteric_A_PVA_/S267K/L368D/K370S/M428L/
N434S SEQ ID NO: 1295
QVQLVQSGAEVKKPGASVKVSCKVSGFSVTSYNVHWVRQAPGQGLEWMGVMWSGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2-IL23-A[IL23]_L1_CH1_IgG1_PVA_/S267K/S364K/E357Q/M428L/N434S SEQ ID NO: 1296
DIQMTQSPSSLSASVGDRVTITCKASRDVAIAVAWYQQKPGKVPKLLIYWASTRHTGVPSRFSGSGSRTDFTLTISSLQPEDVADYF
CHQYSSYPFTFGSGTKLEIKSS/ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPPVAGPSVFLFPPKPKDTLMISRTPEVTCVVVDVKHED
PEVKFNWYVDGVEVHNAKTKPREEQYNSTYRVVSVLTVLHQDWLNGKEYKCKVSNKALPAPIEKTISKAKGQPREPQVYTLPPS
REQMTKNQVKLTCLVKGFYPSDIAVEWESNGQPENNYKTTPPVLDSDGSFFLYSKLTVDKSRWQQGNVFSCSVLHEALHSHYTQ
KSLSLSPGK
Chain 3-041[TL1a]_L1_D122K/E123K Light Chain SEQ ID NO: 1297
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGAGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4-IL23-A[IL23]_H1_Ckappa Light Chain SEQ ID NO: 1298
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAWFIYWGQGTLVTVSS/ASVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
XENP52517-041[TL1a]_H1_Q39E/K213E/K218D_L1_Q38K/D122K/E123K_Fab-IL23-
A[IL23]_L1_Q38K_CH1_H1_Q39E_Ckappa_crossMab_VH_VL-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_H1_Q39E/K213E/K218D_IgG1_pI(-)_Isosteric_A_PVA_/S267K/L368D/K370S/M428L/
N434S SEQ ID NO: 1299
QVQLVQSGAEVKKPGASVKVSCKVSGFSVTSYNVHWVREAPGQGLEWMGVMWSGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVLHEALHSHYTQKSLSLSPGK
Chain 2-IL23-A[IL23]_L1_Q38K_CH1_IgG1_PVA_/S267K/S364K/E357Q/M428L/N434S SEQ ID NO: 1300
DIQMTQSPSSLSASVGDRVTITCKASRDVAIAVAWYQKKPGKVPKLLIYWASTRHTGVPSRFSGSGSRTDFTLTISSLQPEDVADYF
CHQYSSYPFTFGSGTKLEIKSS/ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPPVAGPSVFLFPPKPKDTLMISRTPEVTCVVVDVKHED
PEVKFNWYVDGVEVHNAKTKPREEQYNSTYRVVSVLTVLHQDWLNGKEYKCKVSNKALPAPIEKTISKAKGQPREPQVYTLPPS
REQMTKNQVKLTCLVKGFYPSDIAVEWESNGQPENNYKTTPPVLDSDGSFFLYSKLTVDKSRWQQGNVFSCSVLHEALHSHYTQ
KSLSLSPGK
Chain 3-041[TL1a]_L1_Q38K/D122K/E123K Light Chain SEQ ID NO: 1301
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGAGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4-IL23-A[IL23]_H1_Q39E_Ckappa Light Chain SEQ ID NO: 1302
QVQLVQSGAEVKKPGSSVKVSCKASGYTFTDQTIHWMREAPGQGLEWIGYIYPRDDSPKYNENFKGKVTITADKSTSTAYMELSS
LRSEDTAVYYCAIPDRSGYAWFIYWGQGTLVTVSS/ASVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
XENP52564-IL23-A[IL23]_H1_K213E/K218D_L1_D122K/E123K_Fab-041[TL1a]_L1_CH1_H1_Ckappa_crossMab_
VH_VL-IgG1_pI(-)_Isosteric_A_PVA_/S267K/L368D/K370S/M428L/N434S-IgG1_PVA_/S267K/S364K/
E357Q/M428L/N434S
Chain 1-IL23-A[IL23]_H1_K213E/K218D_IgG1_pI(-)_Isosteric_A_PVA_/S267K/L368D/K370S/M428L/N434S
SEQ ID NO: 1303
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSDTKVDEKVEPDSCDKTHTCPPCPAPPVAGPSVFLFPPKPKDTLMISRT
PEVTCVVVDVKHEDPEVKFNWYVDGVEVHNAKTKPREEEYNSTYRVVSVLTVLHQDWLNGKEYKCKVSNKALPAPIEKTISKA
KGQPREPQVYTLPPSREEMTKNQVSLTCDVSGFYPSDIAVEWESDGQPENNYKTTPPVLDSDGSFFLYSKLTVDKSRWEQGDVFSC
SVLHEALHSHYTQKSLSLSPGK
Chain 2-041[TL1a]_L1_CH1_IgG1_PVA_/S267K/S364K/E357Q/M428L/N434S SEQ ID NO: 1304
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGAGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPPVAGPSVFLFPPKPKDTLMISRTPEVTCVVVDVKHEDP
EVKFNWYVDGVEVHNAKTKPREEQYNSTYRVVSVLTVLHQDWLNGKEYKCKVSNKALPAPIEKTISKAKGQPREPQVYTLPPSR
EQMTKNQVKLTCLVKGFYPSDIAVEWESNGQPENNYKTTPPVLDSDGSFFLYSKLTVDKSRWQQGNVFSCSVLHEALHSHYTQK
SLSLSPGK
Chain 3-IL23-A[IL23]_L1_D122K/E123K Light Chain SEQ ID NO: 1305
DIQMTQSPSSLSASVGDRVTITCKASRDVAIAVAWYQQKPGKVPKLLIYWASTRHTGVPSRFSGSGSRTDFTLTISSLQPEDVADYF
CHQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4-041[TL1a]_H1_Ckappa Light Chain SEQ ID NO: 1306
QVQLVQSGAEVKKPGASVKVSCKVSGFSVTSYNVHWVRQAPGQGLEWMGVMWSGGNTDYNSKFQGRVTMTRDTSTNTVYME
LSSLRSEDTAVYYCARAGRYYHDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
XENP52565-041[TL1a]_L1_CH1_H1_Ckappa_crossMab_VH_VL-IL23-A[IL23]_H1_K213E/K218D_L1_
D122K/E123K_Fab-IgG1_pI(-)_Isosteric_A_PVA_/S267K/L368D/K370S/M428L/N434S-IgG1_PVA_/S267K/
S364K/E357Q/M428L/N434S
Chain 1-041[TL1a]_L1_CH1_IgG1_pI(-)_Isosteric_A_PVA_/S267K/L368D/K370S/M428L/N434S SEQ ID
NO: 1307
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGAGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1_K213E/K218D_IgG1_PVA_/S267K/S364K/E357Q/M428L/N434S SEQ ID NO: 1308
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDEKVEPDSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3-041[TL1a]_H1_Ckappa Light Chain SEQ ID NO: 1309
QVQLVQSGAEVKKPGASVKVSCKVSGFSVTSYNVHWVRQAPGQGLEWMGVMWSGGNTDYNSKFQGRVTMTRDTSTNTVYME
LSSLRSEDTAVYYCARAGRYYHDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1_D122K/E123K Light Chain SEQ ID NO: 1310
DIQMTQSPSSLSASVGDRVTITCKASRDVAIAVAWYQQKPGKVPKLLIYWASTRHTGVPSRFSGSGSRTDFTLTISSLQPEDVADYF
CHQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP52566-041[TL1a]_L1_Q38K_CH1_H1_Q39E_Ckappa_crossMab_VH_VL-IL23-
A[IL23]_H1_Q39E/K213E/K218D_L1_Q38K/D122K/E123K_Fab-IgG1_pI(-)_
Isosteric_A_PVA_/S267K/L368D/K370S/M428L/N434S-IgG1_PVA_/S267K/S364K/E357Q/M428L/N434S
Chain 1-041[TL1a]_L1_Q38K_CH1_IgG1_pI(-)_Isosteric_A_PVA_/S267K/L368D/K370S/M428L/N434S SEQ
ID NO: 1311
DIQMTQSPSSVSASVGDRVTITCRASEDIYSGLAWYQKKPGKSPKLLIYGASSLQDGVPSRFSGSGSGTDYTLTISSLQPEDFATYFC
QEGLKSPYTFGAGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1_Q39E/K213E/K218D_IgG1_PVA_/S267K/S364K/E357Q/M428L/N434S SEQ ID NO: 1312
QVQLVQSGAEVKKPGSSVKVSCKASGYTFTDQTIHWMREAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDEKVEPDSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3-041[TL1a]_H1_Q39E_Ckappa Light Chain SEQ ID NO: 1313
QVQLVQSGAEVKKPGASVKVSCKVSGFSVTSYNVHWVREAPGQGLEWMGVMWSGGNTDYNSKFQGRVTMTRDTSTNTVYME
LSSLRSEDTAVYYCARAGRYYHDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1_Q38K/D122K/E123K Light Chain SEQ ID NO: 1314
DIQMTQSPSSLSASVGDRVTITCKASRDVAIAVAWYQKKPGKVPKLLIYWASTRHTGVPSRFSGSGSRTDFTLTISSLQPEDVADYF
CHQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53090-041[TL1a]_H1_K213E/K218D_L1_D122K/E123K_Fab-IL23-A[IL23]_L1_CH1_H1_
Ckappa_crossMab_VH_VL-IgG1_pI(-)_Isosteric_A_PV_/S267K/L368D/K370S-IgG1_PVA_/S267K/S364K/E357Q
Chain 1-041[TL1a]_H1_K213E/K218D_IgG1_pI(-)_Isosteric_A_PVA_/S267K/L368D/K370S SEQ ID NO: 1315
QVQLVQSGAEVKKPGASVKVSCKVSGFSVTSYNVHWVRQAPGQGLEWMGVMWSGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLMISRTPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVMHEALHNHYTQKSLSLSPGK
Chain 2-IL23-A[IL23]_L1_CH1_IgGI_PVA_/S267K/S364K/E357Q SEQ ID NO: 1316
DIQMTQSPSSLSASVGDRVTITCKASRDVAIAVAWYQQKPGKVPKLLIYWASTRHTGVPSRFSGSGSRTDFTLTISSLQPEDVADYF
CHQYSSYPFTFGSGTKLEIKSS/ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPPVAGPSVFLFPPKPKDTLMISRTPEVTCVVVDVKHED
PEVKFNWYVDGVEVHNAKTKPREEQYNSTYRVVSVLTVLHQDWLNGKEYKCKVSNKALPAPIEKTISKAKGQPREPQVYTLPPS
REQMTKNQVKLTCLVKGFYPSDIAVEWESNGQPENNYKTTPPVLDSDGSFFLYSKLTVDKSRWQQGNVFSCSVMHEALHNHYTQ
KSLSLSPGK
Chain 3-041[TL1a]_L1_D122K/E123K Light Chain SEQ ID NO: 1317
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGAGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4-IL23-A[IL23]_L1_CH1_H1_Ckappa Light Chain SEQ ID NO: 1318
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAWFIYWGQGTLVTVSS/ASVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
XENP52877-041[TL1a]_H1_K147E/K213E_L1_E123R/Q124K_Fab-IL23-A[IL23]_L1_CH1_H1_Ckappa_crossMab_
VH_VL-IgG1_M428L/N434S-IgG1_M428L/N434S
Chain 1-041[TL1a]_H1_K147E/K213E_IgG1_M428L/N434S SEQ ID NO: 2312
QVQLVQSGAEVKKPGASVKVSCKVSGFSVTSYNVHWVRQAPGQGLEWMGVMWSGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVEDYFPEPVTVSW
NSGALTSGVHTFPAVLQSSGLYSLSSVVTVPSSSLGTQTYICNVNHKPSNTKVDEKVEPKSCDKTHTCPPCPAPEAAGGPSVFLFPP
KPKDTLMISRTPEVTCVVVDVSHEDPEVKFNWYVDGVEVHNAKTKPREEQYNSTYRVVSVLTVLHQDWLNGKEYKCKVSNKAL
GAPIEKTISKAKGQPREPQVCTLPPSRDELTKNQVSLSCAVKGFYPSDIAVEWESNGQPENNYKTTPPVLDSDGSFFLVSKLTVDKS
RWQQGNVFSCSVLHEALHSHYTQKSLSLSPGK
Chain 2-IL23-A[IL23]_L1_IgG1_M428L/N434S SEQ ID NO: 2313
DIQMTQSPSSLSASVGDRVTITCKASRDVAIAVAWYQQKPGKVPKLLIYWASTRHTGVPSRFSGSGSRTDFTLTISSLQPEDVADYF
CHQYSSYPFTFGSGTKLEIKSS/ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALGAPIEKTISKAKGQPREPQVYTLPP
CRDELTKNQVSLWCLVKGFYPSDIAVEWESNGQPENNYKTTPPVLDSDGSFFLYSKLTVDKSRWQQGNVFSCSVLHEALHSHYTQ
KSLSLSPGK
Chain 3-041[TL1a]_L1_E123R/Q124K Light Chain SEQ ID NO: 2314
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGAGTKLEIK/RTVAAPSVFIFPPSDRKLKSGTASVVCLLNNFYPREAKVQWKVDNALQSGNSQESVTEQDSKDSTYS
LSSTLTLSKADYEKHKVYACEVTHQGLSSPVTKSFNRGEC
Chain 4-IL23-A[IL23]_H1_Ckappa Light Chain SEQ ID NO: 2315
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAWFIYWGQGTLVTVSS/ASVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
XENP53091-041[TL1a]_H1_K213E/K218D_L1_D122K/E123K_Fab-IL23-A[IL23]_L1_CH1_H1_Ckappa_crossMab_
VH_VL-IgG1_pI(-)_Isosteric_A_PVA_/M252Y/S254T/T256E/S267K/L368D/K370S-
IgG1_PVA_/M252Y/S254T/T256E/S267K/S364K/E357Q
Chain 1-041[TL1a]_H1_K213E/K218D_IgG1_pI(-)_Isosteric_A_PVA_/M252Y/S254T/T256E/S267K/L368D/K370S
SEQ ID NO: 1319
QVQLVQSGAEVKKPGASVKVSCKVSGFSVTSYNVHWVRQAPGQGLEWMGVMWSGGNTDYNSKFQGRVTMTRDTSTNTVYME
LSSLRSEDTAVYYCARAGRYYHDSNYYWDFPYWGQGTLVTVSS/ASTKGPSVFPLAPSSKSTSGGTAALGCLVKDYFPEPVTVSW
NSGALTSGVHTFPAVLQSSGLYSLSSVVTVPSSSLGTQTYICNVNHKPSDTKVDEKVEPDSCDKTHTCPPCPAPPVAGPSVFLFPPKP
KDTLYITREPEVTCVVVDVKHEDPEVKFNWYVDGVEVHNAKTKPREEEYNSTYRVVSVLTVLHQDWLNGKEYKCKVSNKALPA
PIEKTISKAKGQPREPQVYTLPPSREEMTKNQVSLTCDVSGFYPSDIAVEWESDGQPENNYKTTPPVLDSDGSFFLYSKLTVDKSRW
EQGDVFSCSVMHEALHNHYTQKSLSLSPGK
Chain 2-IL23-A[IL23]_L1_CH1_IgGI_PVA_/M252Y/S254T/T256E/S267K/S364K/E357Q SEQ ID NO: 1320
DIQMTQSPSSLSASVGDRVTITCKASRDVAIAVAWYQQKPGKVPKLLIYWASTRHTGVPSRFSGSGSRTDFTLTISSLQPEDVADYF
CHQYSSYPFTFGSGTKLEIKSS/ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPPVAGPSVFLFPPKPKDTLYITREPEVTCVVVDVKHED
PEVKFNWYVDGVEVHNAKTKPREEQYNSTYRVVSVLTVLHQDWLNGKEYKCKVSNKALPAPIEKTISKAKGQPREPQVYTLPPS
REQMTKNQVKLTCLVKGFYPSDIAVEWESNGQPENNYKTTPPVLDSDGSFFLYSKLTVDKSRWQQGNVFSCSVMHEALHNHYTQ
KSLSLSPGK
Chain 3-041[TL1a]_L1_D122K/E123K Light Chain SEQ ID NO: 1321
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGAGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
Chain 4-IL23-A[IL23]_L1_CH1_H1_Ckappa Light Chain SEQ ID NO: 1322
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAWFIYWGQGTLVTVSS/ASVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
XENP53381-041[TL1a]_L1.27_CH1_H1.324_Ckappa_crossMab_VH_VL-IL23-
A[IL23]_H1_K213E/K218D_L1.117_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_L1.27_CH1_IgG1_pI(-)_Isosteric_A_PVA_/S267K/L368D/K370S/M428L/N434S SEQ ID
NO: 1323
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1_K213E/K218D_IgG1_PVA_/S267K/S364K/E357Q/M428L/N434S SEQ ID NO: 1324
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDEKVEPDSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3-041[TL1a]_H1.324_Ckappa Light Chain SEQ ID NO: 1325
QVQLVQSGAEVKKPGASVKVSCKVSGFPVTSYNVHWVRQAPGQGLEWMGVMWYGGNTDYNSKFQGRVTMTRDTSTNTVYME
LSSLRSEDTAVYYCARAGRYYHDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.117_D122K/E123K Light Chain SEQ ID NO: 1326
DIQMTQSPSSLSASVGDRVTITCKASRDVAIAVAWYQQKPGKVPKLLIYWASVRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53382-041[TL1a]_L1.27_CH1_H1.324_Ckappa_crossMab_VH_VL-IL23-
A[IL23]_H1.187_K213E/K218D_L1.116_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_L1.27_CH1_IgG1_pl(-)_Isosteric_A_PVA_/S267K/L368D/K370S/M428L/N434S SEQ ID
NO: 1327
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1.187_K213E/K218D_IgG1_PVA_/S267K/S364K/E357Q/M428L/N434S SEQ ID NO: 1328
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAFFIYWGQGTLVTVSS/ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDEKVEPDSCDKTHTCPPCPAPPVAGPSVFLFPPKPKDTLMISRTP
EVTCVVVDVKHEDPEVKFNWYVDGVEVHNAKTKPREEQYNSTYRVVSVLTVLHQDWLNGKEYKCKVSNKALPAPIEKTISKAK
GQPREPQVYTLPPSREQMTKNQVKLTCLVKGFYPSDIAVEWESNGQPENNYKTTPPVLDSDGSFFLYSKLTVDKSRWQQGNVFSC
SVLHEALHSHYTQKSLSLSPGK
Chain 3-041[TL1a]_H1.324_Ckappa Light Chain SEQ ID NO: 1329
QVQLVQSGAEVKKPGASVKVSCKVSGFPVTSYNVHWVRQAPGQGLEWMGVMWYGGNTDYNSKFQGRVTMTRDTSTNTVYME
LSSLRSEDTAVYYCARAGRYYHDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.116_D122K/E123K Light Chain SEQ ID NO: 1330
DIQMTQSPSSLSASVGDRVTITCKASRDVAIAVAWYQQKPGKVPKLLIYWASQRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53383-041[TL1a]_L1.27_CH1_H1.324_Ckappa_crossMab_VH_VL-IL23-
A[IL23]_H1.41_K213E/K218D_L1.117_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_L1.27_CH1_IgGI_pI(-)_Isosteric_A_PVA_/S267K/L368D/K370S/M428L/N434S SEQ ID
NO: 1331
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1.41_K213E/K218D_IgG1_PVA_/S267K/S364K/E357Q/M428L/N434S SEQ ID NO: 1332
QVQLVQSGAEVKKPGSSVKVSCKASGYTFTDQTLHWMRQAPGQGLEWIGYIYPRDDSPKYNENFKGKVTITADKSTSTAYMELS
SLRSEDTAVYYCAIPDRSGYAWFIYWGQGTLVTVSS/ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDEKVEPDSCDKTHTCPPCPAPPVAGPSVFLFPPKPKDTLMISR
TPEVTCVVVDVKHEDPEVKFNWYVDGVEVHNAKTKPREEQYNSTYRVVSVLTVLHQDWLNGKEYKCKVSNKALPAPIEKTISK
AKGQPREPQVYTLPPSREQMTKNQVKLTCLVKGFYPSDIAVEWESNGQPENNYKTTPPVLDSDGSFFLYSKLTVDKSRWQQGNVF
SCSVLHEALHSHYTQKSLSLSPGK
Chain 3-041[TL1a]_H1.324_Ckappa Light Chain SEQ ID NO: 1333
QVQLVQSGAEVKKPGASVKVSCKVSGFPVTSYNVHWVRQAPGQGLEWMGVMWYGGNTDYNSKFQGRVTMTRDTSTNTVYME
LSSLRSEDTAVYYCARAGRYYHDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.117_D122K/E123K Light Chain SEQ ID NO: 1334
DIQMTQSPSSLSASVGDRVTITCKASRDVAIAVAWYQQKPGKVPKLLIYWASVRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53384-041[TL1a]_L1.27_CH1_H1.318_Ckappa_crossMab_VH_VL-IL23-
A[IL23]_H1_K213E/K218D_L1.117_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_L1.27_CH1_IgG1_pI(-)_Isosteric_A_PVA_/S267K/L368D/K370S/M428L/N434S SEQ ID
NO: 1335
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1_K213E/K218D_IgG1_PVA_/S267K/S364K/E357Q/M428L/N434S SEQ ID NO: 1336
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDEKVEPDSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3-041[TL1a]_H1.318_Ckappa Light Chain SEQ ID NO: 1337
QVQLVQSGAEVKKPGASVKVSCKVSGFSVTSYNVHWVRQAPGQGLEWMGVMWFGGNTDYNSKFQGRVTMTRDTSTNTVYME
LSSLRSEDTAVYYCARAGRYYHDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.117_D122K/E123K Light Chain SEQ ID NO: 1338
DIQMTQSPSSLSASVGDRVTITCKASRDVAIAVAWYQQKPGKVPKLLIYWASVRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53385-041[TL1a]_L1.27_CH1_H1.318_Ckappa_crossMab_VH_VL-IL23-
A[IL23]_H1.187_K213E/K218D_L1.116_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_L1.27_CH1_IgG1_pI(-)_Isosteric_A_PVA_/S267K/L368D/K370S/M428L/N434S SEQ ID
NO: 1339
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1.187_K213E/K218D_IgG1_PVA_/S267K/S364K/E357Q/M428L/N434S SEQ ID NO: 1340
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAFFIYWGQGTLVTVSS/ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDEKVEPDSCDKTHTCPPCPAPPVAGPSVFLFPPKPKDTLMISRTP
EVTCVVVDVKHEDPEVKFNWYVDGVEVHNAKTKPREEQYNSTYRVVSVLTVLHQDWLNGKEYKCKVSNKALPAPIEKTISKAK
GQPREPQVYTLPPSREQMTKNQVKLTCLVKGFYPSDIAVEWESNGQPENNYKTTPPVLDSDGSFFLYSKLTVDKSRWQQGNVFSC
SVLHEALHSHYTQKSLSLSPGK
Chain 3-041[TL1a]_H1.318_Ckappa Light Chain SEQ ID NO: 1341
QVQLVQSGAEVKKPGASVKVSCKVSGFSVTSYNVHWVRQAPGQGLEWMGVMWFGGNTDYNSKFQGRVTMTRDTSTNTVYME
LSSLRSEDTAVYYCARAGRYYHDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.116_D122K/E123K Light Chain SEQ ID NO: 1342
DIQMTQSPSSLSASVGDRVTITCKASRDVAIAVAWYQQKPGKVPKLLIYWASQRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53386-041[TL1a]_L1.27_CH1_H1.318_Ckappa_crossMab_VH_VL-IL23-
A[IL23]_H1.41_K213E/K218D_L1.117_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_L1.27_CH1_IgG1_pI(-)_Isosteric_A_PVA_/S267K/L368D/K370S/M428L/N434S SEQ ID
NO: 1343
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1.41_K213E/K218D_IgG1_PVA_/S267K/S364K/E357Q/M428L/N434S SEQ ID NO: 1344
QVQLVQSGAEVKKPGSSVKVSCKASGYTFTDQTLHWMRQAPGQGLEWIGYIYPRDDSPKYNENFKGKVTITADKSTSTAYMELS
SLRSEDTAVYYCAIPDRSGYAWFIYWGQGTLVTVSS/ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDEKVEPDSCDKTHTCPPCPAPPVAGPSVFLFPPKPKDTLMISR
TPEVTCVVVDVKHEDPEVKFNWYVDGVEVHNAKTKPREEQYNSTYRVVSVLTVLHQDWLNGKEYKCKVSNKALPAPIEKTISK
AKGQPREPQVYTLPPSREQMTKNQVKLTCLVKGFYPSDIAVEWESNGQPENNYKTTPPVLDSDGSFFLYSKLTVDKSRWQQGNVF
SCSVLHEALHSHYTQKSLSLSPGK
Chain 3-041[TL1a]_H1.318_Ckappa Light Chain SEQ ID NO: 1345
QVQLVQSGAEVKKPGASVKVSCKVSGFSVTSYNVHWVRQAPGQGLEWMGVMWFGGNTDYNSKFQGRVTMTRDTSTNTVYME
LSSLRSEDTAVYYCARAGRYYHDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.117_D122K/E123K Light Chain SEQ ID NO: 1346
DIQMTQSPSSLSASVGDRVTITCKASRDVAIAVAWYQQKPGKVPKLLIYWASVRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENE53387-041[TL1a]_L1.27_CH1_H1.447_Ckapa_crossMab_VH_VL-IL23-
A[IL23]_H1_K213F/K218D_L1.117_D122K/E123K_Fab-IgG1_pI(-)_Isoteric_A_PVA_S267K/L368D/K370S/M428L/
N434S-IgG1_PVA_/S267K/S364K/E357Q/M428L/N434S
Chain 1-041[TL1a]_L1.27_CH1_IgG1_pI(-)_Isosteric_A_PVA_/S267K/L368D/K370S/M428L/N434S SEQ ID
NO: 1347
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1_K213E/K218D_IgG1_PVA_/S267K/S364K/E357Q/M428L/N434S SEQ ID NO: 1348
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDEKVEPDSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3-041[TL1a]_H1.447_Ckappa Light Chain SEQ ID NO: 1349
QVQLVQSGAEVKKPGASVKVSCKVSGFSVTRYNVHWVRQAPGQGLEWMGVMWSGANTDYNSKFQGRVTMTRDTSTNTVYME
LSSLRSEDTAVYYCARAGRYYVDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.117_D122K/E123K Light Chain SEQ ID NO: 1350
DIQMTQSPSSLSASVGDRVTITCKASRDVAIAVAWYQQKPGKVPKLLIYWASVRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53388-041[TL1a]_L1.27_CH1_H1.447_Ckappa_crossMab_VH_VL-IL23-
A[IL23]_H1.187_K213E/K218D_L1.116_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_L1.27_CH1_IgG1_pI(-)_Isosteric_A_PVA_/S267K/L368D/K370S/M428L/N434S SEQ ID
NO: 1351
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1.187_K213E/K218D_IgG1_PVA_/S267K/S364K/E357Q/M428L/N434S SEQ ID NO: 1352
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAFFIYWGQGTLVTVSS/ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDEKVEPDSCDKTHTCPPCPAPPVAGPSVFLFPPKPKDTLMISRTP
EVTCVVVDVKHEDPEVKFNWYVDGVEVHNAKTKPREEQYNSTYRVVSVLTVLHQDWLNGKEYKCKVSNKALPAPIEKTISKAK
GQPREPQVYTLPPSREQMTKNQVKLTCLVKGFYPSDIAVEWESNGQPENNYKTTPPVLDSDGSFFLYSKLTVDKSRWQQGNVFSC
SVLHEALHSHYTQKSLSLSPGK
Chain 3-041[TL1a]_H1.447_Ckappa Light Chain SEQ ID NO: 1353
QVQLVQSGAEVKKPGASVKVSCKVSGFSVTRYNVHWVRQAPGQGLEWMGVMWSGANTDYNSKFQGRVTMTRDTSTNTVYME
LSSLRSEDTAVYYCARAGRYYVDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.116_D122K/E123K Light Chain SEQ ID NO: 1354
DIQMTQSPSSLSASVGDRVTITCKASRDVAIAVAWYQQKPGKVPKLLIYWASQRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53389-041[TL1a]_L1.27_CH1_H1.447_Ckappa_crossMab_VH_VL-IL23-
A[IL23]_H1.41_K213E/K218D_L1.117_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_L1.27_CH1_IgG1_pI(-)_Isosteric_A_PVA_/S267K/L368D/K370S/M428L/N434S SEQ ID
NO: 1355
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1.41_K213E/K218D_IgG1_PVA_/S267K/S364K/E357Q/M428L/N434S SEQ ID NO: 1356
QVQLVQSGAEVKKPGSSVKVSCKASGYTFTDQTLHWMRQAPGQGLEWIGYIYPRDDSPKYNENFKGKVTITADKSTSTAYMELS
SLRSEDTAVYYCAIPDRSGYAWFIYWGQGTLVTVSS/ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDEKVEPDSCDKTHTCPPCPAPPVAGPSVFLFPPKPKDTLMISR
TPEVTCVVVDVKHEDPEVKFNWYVDGVEVHNAKTKPREEQYNSTYRVVSVLTVLHQDWLNGKEYKCKVSNKALPAPIEKTISK
AKGQPREPQVYTLPPSREQMTKNQVKLTCLVKGFYPSDIAVEWESNGQPENNYKTTPPVLDSDGSFFLYSKLTVDKSRWQQGNVF
SCSVLHEALHSHYTQKSLSLSPGK
Chain 3-041[TL1a]_H1.447_Ckappa Light Chain SEQ ID NO: 1357
QVQLVQSGAEVKKPGASVKVSCKVSGFSVTRYNVHWVRQAPGQGLEWMGVMWSGANTDYNSKFQGRVTMTRDTSTNTVYME
LSSLRSEDTAVYYCARAGRYYVDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.117_D122K/E123K Light Chain SEQ ID NO: 1358
DIQMTQSPSSLSASVGDRVTITCKASRDVAIAVAWYQQKPGKVPKLLIYWASVRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53390-041[TL1a]_L1.27_CH1_H1.239_Ckappa_crossMab_VH_VL-IL23-
A[IL23]_H1_K213E/K218D_L1.117_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_L1.27_CH1_IgG1_pI(-)_Isosteric_A_PVA_/S267K/L368D/K370S/M428L/N434S SEQ ID
NO: 1359
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1_K213E/K218D_IgG1_PVA_/S267K/S364K/E357Q/M428L/N434S SEQ ID NO: 1360
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDEKVEPDSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3-041[TL1a]_H1.239_Ckappa Light Chain SEQ ID NO: 1361
QVQLVQSGAEVKKPGASVKVSCKVSGFSVTSYNVHWVRQAPGQGLEWMGVMWRGGNTDYNSKFQGRVTMTRDTSTNTVYME
LSSLRSEDTAVYYCARAGRYYHDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.117_D122K/E123K Light Chain SEQ ID NO: 1362
DIQMTQSPSSLSASVGDRVTITCKASRDVAIAVAWYQQKPGKVPKLLIYWASVRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53391-041[TL1a]_L1.27_CH1_H1.239_Ckappa_crossMab_VH_VL-IL23-
A[IL23]_H1.187_K213E/K218D_L1.116_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_L1.27_CH1_IgG1_pl(-)_Isosteric_A_PVA_/S267K/L368D/K370S/M428L/N434S SEQ ID
NO: 1363
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1.187_K213E/K218D_IgG1_PVA_/S267K/S364K/E357Q/M428L/N434S SEQ ID NO: 1364
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAFFIYWGQGTLVTVSS/ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDEKVEPDSCDKTHTCPPCPAPPVAGPSVFLFPPKPKDTLMISRTP
EVTCVVVDVKHEDPEVKFNWYVDGVEVHNAKTKPREEQYNSTYRVVSVLTVLHQDWLNGKEYKCKVSNKALPAPIEKTISKAK
GQPREPQVYTLPPSREQMTKNQVKLTCLVKGFYPSDIAVEWESNGQPENNYKTTPPVLDSDGSFFLYSKLTVDKSRWQQGNVFSC
SVLHEALHSHYTQKSLSLSPGK
Chain 3-041[TL1a]_H1.239_Ckappa Light Chain SEQ ID NO: 1365
QVQLVQSGAEVKKPGASVKVSCKVSGFSVTSYNVHWVRQAPGQGLEWMGVMWRGGNTDYNSKFQGRVTMTRDTSTNTVYME
LSSLRSEDTAVYYCARAGRYYHDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.116_D122K/E123K Light Chain SEQ ID NO: 1366
DIQMTQSPSSLSASVGDRVTITCKASRDVAIAVAWYQQKPGKVPKLLIYWASQRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53392-041[TL1a]_L1.27_CH1_H1.239_Ckappa_crossMab_VH_VL-IL23-
A[IL23]_H1.41_K213E/K218D_L1.117_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_L1.27_CH1_IgG1_pI(-)_Isosteric_A_PVA_/S267K/L368D/K370S/M428L/N434S SEQ ID
NO: 1367
DIQMTQSPSSVSASVGDRVTITCRASEDIYSGLAWYQQKPGKSPKLLIYGASSLQDGVPSRESGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1.41_K213E/K218D_IgG1_PVA_/S267K/S364K/E357Q/M428L/N434S SEQ ID NO: 1368
QVQLVQSGAEVKKPGSSVKVSCKASGYTFTDQTLHWMRQAPGQGLEWIGYIYPRDDSPKYNENFKGKVTITADKSTSTAYMELS
SLRSEDTAVYYCAIPDRSGYAWFIYWGQGTLVTVSS/ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDEKVEPDSCDKTHTCPPCPAPPVAGPSVFLFPPKPKDTLMISR
TPEVTCVVVDVKHEDPEVKFNWYVDGVEVHNAKTKPREEQYNSTYRVVSVLTVLHQDWLNGKEYKCKVSNKALPAPIEKTISK
AKGQPREPQVYTLPPSREQMTKNQVKLTCLVKGFYPSDIAVEWESNGQPENNYKTTPPVLDSDGSFFLYSKLTVDKSRWQQGNVF
SCSVLHEALHSHYTQKSLSLSPGK
Chain 3-041[TL1a]_H1.239_Ckappa Light Chain SEQ ID NO: 1369
QVQLVQSGAEVKKPGASVKVSCKVSGFSVTSYNVHWVRQAPGQGLEWMGVMWRGGNTDYNSKFQGRVTMTRDTSTNTVYME
LSSLRSEDTAVYYCARAGRYYHDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.117_D122K/E123K Light Chain SEQ ID NO: 1370
DIQMTQSPSSLSASVGDRVTITCKASRDVAIAVAWYQQKPGKVPKLLIYWASVRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53415-041[TL1a]_L1.27_CH1_H1.337_Ckappa_crossMab-VH_VL-IL23-
A[IL23]_H1_K213E/K218D_L1.117_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_L1.27_CH1_IgG1_pI(-)_Isosteric_A_PVA_/S267K/L368D/K370S/M428L/N434S SEQ ID
NO: 1371
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1_K213E/K218D_IgG1_PVA_/S267K/S364K/E357Q/M428L/N434S SEQ ID NO: 1372
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDEKVEPDSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3-041[TL1a]_H1.337_Ckappa Light Chain SEQ ID NO: 1373
QVQLVQSGAEVKKPGASVKVSCKVSGFSVTSYNVHWVRQAPGQGLEWMGVMWYGGNTDYNSKFQGRVTMTRDTSTNTVYME
LSSLRSEDTAVYYCARAGRYYVDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.117_D122K/E123K Light Chain SEQ ID NO: 1374
DIQMTQSPSSLSASVGDRVTITCKASRDVAIAVAWYQQKPGKVPKLLIYWASVRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53416-041[TL1a]_L1.27_CH1_H1.337_Ckappa_crossMab_VH_VL-IL23-
A[IL23]_H1.187_K213E/K218D_L1.116_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_L1.27_CH1_IgG1_pI(-)_Isosteric_A_PVA_/S267K/L368D/K370S/M428L/N434S SEQ ID
NO: 1375
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1.187_K213E/K218D_IgG1_PVA_/S267K/S364K/E357Q/M428L/N434S SEQ ID NO: 1376
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAFFIYWGQGTLVTVSS/ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDEKVEPDSCDKTHTCPPCPAPPVAGPSVFLFPPKPKDTLMISRTP
EVTCVVVDVKHEDPEVKFNWYVDGVEVHNAKTKPREEQYNSTYRVVSVLTVLHQDWLNGKEYKCKVSNKALPAPIEKTISKAK
GQPREPQVYTLPPSREQMTKNQVKLTCLVKGFYPSDIAVEWESNGQPENNYKTTPPVLDSDGSFFLYSKLTVDKSRWQQGNVFSC
SVLHEALHSHYTQKSLSLSPGK
Chain 3-041[TL1a]_H1.337_Ckappa Light Chain SEQ ID NO: 1377
QVQLVQSGAEVKKPGASVKVSCKVSGFSVTSYNVHWVRQAPGQGLEWMGVMWYGGNTDYNSKFQGRVTMTRDTSTNTVYME
LSSLRSEDTAVYYCARAGRYYVDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.116_D122K/E123K Light Chain SEQ ID NO: 1378
DIQMTQSPSSLSASVGDRVTITCKASRDVAIAVAWYQQKPGKVPKLLIYWASQRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP53417-041[TL1a]_L1.27_CH1_H1.337_Ckappa_crossMab_VH_VL-IL23-
A[IL23]_H1.41_K213E/K218D_L1.117_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-041[TL1a]_L1.27_CH1_IgG1_pI(-)_Isosteric_A_PVA_/S267K/L368D/K370S/M428L/N434S SEQ ID
NO: 1379
DIQMTQSPSSVSASVGDRVTITCRASEDIYSGLAWYQQKPGKSPKLLIYGASSLQDGVPSRFSGSGSGTDYTLTISSLQPEDFATYFC
QEGLKSPYTFGQGTKLEIKSS/ASTKGPSVFPLAPSSKSTSGGTAALGCLVKDYFPEPVTVSWNSGALTSGVHTFPAVLQSSGLYSLS
SVVTVPSSSLGTQTYICNVNHKPSDTKVDKKVEPKSCDKTHTCPPCPAPPVAGPSVFLFPPKPKDTLMISRTPEVTCVVVDVKHEDP
EVKFNWYVDGVEVHNAKTKPREEEYNSTYRVVSVLTVLHQDWLNGKEYKCKVSNKALPAPIEKTISKAKGQPREPQVYTLPPSR
EEMTKNQVSLTCDVSGFYPSDIAVEWESDGQPENNYKTTPPVLDSDGSFFLYSKLTVDKSRWEQGDVFSCSVLHEALHSHYTQKS
LSLSPGK
Chain 2-IL23-A[IL23]_H1.41_K213E/K218D_IgG1_PVA_/S267K/S364K/E357Q/M428L/N434S SEQ ID NO: 1380
QVQLVQSGAEVKKPGSSVKVSCKASGYTFTDQTLHWMRQAPGQGLEWIGYIYPRDDSPKYNENFKGKVTITADKSTSTAYMELS
SLRSEDTAVYYCAIPDRSGYAWFIYWGQGTLVTVSS/ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDEKVEPDSCDKTHTCPPCPAPPVAGPSVFLFPPKPKDTLMISR
TPEVTCVVVDVKHEDPEVKFNWYVDGVEVHNAKTKPREEQYNSTYRVVSVLTVLHQDWLNGKEYKCKVSNKALPAPIEKTISK
AKGQPREPQVYTLPPSREQMTKNQVKLTCLVKGFYPSDIAVEWESNGQPENNYKTTPPVLDSDGSFFLYSKLTVDKSRWQQGNVF
SCSVLHEALHSHYTQKSLSLSPGK
Chain 3-041[TL1a]_H1.337_Ckappa Light Chain SEQ ID NO: 1381
QVQLVQSGAEVKKPGASVKVSCKVSGFSVTSYNVHWVRQAPGQGLEWMGVMWYGGNTDYNSKFQGRVTMTRDTSTNTVYME
LSSLRSEDTAVYYCARAGRYYVDSNYYWDFPYWGQGTLVTVSS/ASVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Chain 4-IL23-A[IL23]_L1.117_D122K/E123K Light Chain SEQ ID NO: 1382
DIQMTQSPSSLSASVGDRVTITCKASRDVAIAVAWYQQKPGKVPKLLIYWASVRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP55905-70417_111[TL1a]_L1_CH1_H1_Ckappa_crossMab_VH_VL-IL23-
A[IL23]_H1_K213E/K218D_L1_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-70417_111[TL1a]_L1_CH1_IgG1_pI(-)_Isosteric_A_PVA_/S267K/L368D/K370S/M428L/N434S SEQ
ID NO: 1383
DIVMTQSPDSLAVSLGERATINCKASQDVSTAVVWYQQKPGQSPKLLIYWASTRHTGVPDRFTGSGSGTDYTLTISSLQAEDVAVY
HCQQHYSTPYTFGGGTKLEIKSS/ASTKGPSVFPLAPSSKSTSGGTAALGCLVKDYFPEPVTVSWNSGALTSGVHTFPAVLQSSGLY
SLSSVVTVPSSSLGTQTYICNVNHKPSDTKVDKKVEPKSCDKTHTCPPCPAPPVAGPSVFLFPPKPKDTLMISRTPEVTCVVVDVKH
EDPEVKFNWYVDGVEVHNAKTKPREEEYNSTYRVVSVLTVLHQDWLNGKEYKCKVSNKALPAPIEKTISKAKGQPREPQVYTLP
PSREEMTKNQVSLTCDVSGFYPSDIAVEWESDGQPENNYKTTPPVLDSDGSFFLYSKLTVDKSRWEQGDVFSCSVLHEALHSHYT
QKSLSLSPGK
Chain 2-IL23-A[IL23]_H1_K213E/K218D_IgG1_PVA_/S267K/S364K/E357Q/M428L/N434S SEQ ID NO: 1384
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDEKVEPDSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3-70417_111[TL1a]_H1_Ckappa Light Chain SEQ ID NO: 1385
EVQLVESGGGLVQPGGSLRLSCAASGFTFSSFGMHWVRQAPGKGLEWVSYISSGSTTIYYADSVKGRFTISRDNAKNSLYLQMNS
LRAEDTAVYYCARAPLLRGAMDYWGQGTLVTVSS/ASVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
Chain 4-IL23-A[IL23]_L1_D122K/E123K Light Chain SEQ ID NO: 1386
DIQMTQSPSSLSASVGDRVTITCKASRDVAIAVAWYQQKPGKVPKLLIYWASTRHTGVPSRFSGSGSRTDFTLTISSLQPEDVADYF
CHQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP55906-70417_111[TL1a]_L1_CH1_H1_Ckappa_crossMab_VH_VL-IL23-
A[IL23]_H1_K213E/K218D_L1.117_D122K/E123K_Fab-IgG1_pI(-)_Isosteric_A_PVA_/S267K/
L368D/K370S/M428L/N434S-IgG1_PVA_/S267K/S364K/E357Q/M428L/N434S
Chain 1-70417_111[TL1a]_L1_CH1_IgG1_pI(-)_Isosteric_A_PVA_/S267K/L368D/K370S/M428L/N434S SEQ
ID NO: 1387
DIVMTQSPDSLAVSLGERATINCKASQDVSTAVVWYQQKPGQSPKLLIYWASTRHTGVPDRFTGSGSGTDYTLTISSLQAEDVAVY
HCQQHYSTPYTFGGGTKLEIKSS/ASTKGPSVFPLAPSSKSTSGGTAALGCLVKDYFPEPVTVSWNSGALTSGVHTFPAVLQSSGLY
SLSSVVTVPSSSLGTQTYICNVNHKPSDTKVDKKVEPKSCDKTHTCPPCPAPPVAGPSVFLFPPKPKDTLMISRTPEVTCVVVDVKH
EDPEVKFNWYVDGVEVHNAKTKPREEEYNSTYRVVSVLTVLHQDWLNGKEYKCKVSNKALPAPIEKTISKAKGQPREPQVYTLP
PSREEMTKNQVSLTCDVSGFYPSDIAVEWESDGQPENNYKTTPPVLDSDGSFFLYSKLTVDKSRWEQGDVFSCSVLHEALHSHYT
QKSLSLSPGK
Chain 2-IL23-A[IL23]_H1_K213E/K218D_IgG1_PVA_/S267K/S364K/E357Q/M428L/N434S SEQ ID NO: 1388
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAWFIYWGQGTLVTVSS/ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDEKVEPDSCDKTHTCPPCPAPPVAGPSVFLFPPKPKDTLMISRT
PEVTCVVVDVKHEDPEVKFNWYVDGVEVHNAKTKPREEQYNSTYRVVSVLTVLHQDWLNGKEYKCKVSNKALPAPIEKTISKA
KGQPREPQVYTLPPSREQMTKNQVKLTCLVKGFYPSDIAVEWESNGQPENNYKTTPPVLDSDGSFFLYSKLTVDKSRWQQGNVFS
CSVLHEALHSHYTQKSLSLSPGK
Chain 3-70417_111[TL1a]_H1_Ckappa Light Chain SEQ ID NO: 1389
EVQLVESGGGLVQPGGSLRLSCAASGFTFSSFGMHWVRQAPGKGLEWVSYISSGSTTIYYADSVKGRFTISRDNAKNSLYLQMNS
LRAEDTAVYYCARAPLLRGAMDYWGQGTLVTVSS/ASVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
Chain 4-IL23-A[IL23]_L1.117_D122K/E123K Light Chain SEQ ID NO: 1390
DIQMTQSPSSLSASVGDRVTITCKASRDVAIAVAWYQQKPGKVPKLLIYWASVRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC
XENP55907-70417_111[TL1a]_L1_CH1_H1_Ckappa_crossMab_VH_VL-IL23-
A[IL23]_H1.187_K213E/K218D_L1.116_D122K/E123K_Fab-IgG1_pI(-)_Isosteric_A-PVA_/S267K/
L368D/K370S/M428L/N434S-IgG1_PVA_/S267K/S364K/E357Q/M428L/N434S
Chain 1-70417_111[TL1a]_Ll_CH1_IgG1_pI(-)_Isosteric_A_PVA_/S267K/L368D/K370S/M428L/N434S SEQ
ID NO: 1391
DIVMTQSPDSLAVSLGERATINCKASQDVSTAVVWYQQKPGQSPKLLIYWASTRHTGVPDRFTGSGSGTDYTLTISSLQAEDVAVY
HCQQHYSTPYTFGGGTKLEIKSS/ASTKGPSVFPLAPSSKSTSGGTAALGCLVKDYFPEPVTVSWNSGALTSGVHTFPAVLQSSGLY
SLSSVVTVPSSSLGTQTYICNVNHKPSDTKVDKKVEPKSCDKTHTCPPCPAPPVAGPSVFLFPPKPKDTLMISRTPEVTCVVVDVKH
EDPEVKFNWYVDGVEVHNAKTKPREEEYNSTYRVVSVLTVLHQDWLNGKEYKCKVSNKALPAPIEKTISKAKGQPREPQVYTLP
PSREEMTKNQVSLTCDVSGFYPSDIAVEWESDGQPENNYKTTPPVLDSDGSFFLYSKLTVDKSRWEQGDVFSCSVLHEALHSHYT
QKSLSLSPGK
Chain 2-IL23-A[IL23]_H1.187_K213E/K218D_IgG1_PVA_/S267K/S364K/E357Q/M428L/N434S SEQ ID NO: 1392
QVQLVQSGAEVKKPGSSVKVSCKASGYTFTDQTIHWMRQAPGQGLEWIGYIYPRDDSPKYNENFKGKVTITADKSTSTAYMELSS
LRSEDTAVYYCAIPDRSGYAFFIYWGQGTLVTVSS/ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDEKVEPDSCDKTHTCPPCPAPPVAGPSVFLFPPKPKDTLMISRTP
EVTCVVVDVKHEDPEVKFNWYVDGVEVHNAKTKPREEQYNSTYRVVSVLTVLHQDWLNGKEYKCKVSNKALPAPIEKTISKAK
GQPREPQVYTLPPSREQMTKNQVKLTCLVKGFYPSDIAVEWESNGQPENNYKTTPPVLDSDGSFFLYSKLTVDKSRWQQGNVFSC
SVLHEALHSHYTQKSLSLSPGK
Chain 3-70417_111[TL1a]_H1_Ckappa Light Chain SEQ ID NO: 1393
EVQLVESGGGLVQPGGSLRLSCAASGFTFSSFGMHWVRQAPGKGLEWVSYISSGSTTIYYADSVKGRFTISRDNAKNSLYLQMNS
LRAEDTAVYYCARAPLLRGAMDYWGQGTLVTVSS/ASVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
Chain 4-IL23-A[IL23]_L1.116_D122K/E123K Light Chain SEQ ID NO: 1394
DIQMTQSPSSLSASVGDRVTITCKASRDVAIAVAWYQQKPGKVPKLLIYWASQRHTGVPSRFSGSGSRTDFTLTISSLQPEDVADYF
CYQYSSYPFTFGSGTKLEIK/RTVAAPSVFIFPPSKKQLKSGTASVVCLLNNFYPREAKVQWKVDNALQSGNSQESVTEQDSKDSTY
SLSSTLTLSKADYEKHKVYACEVTHQGLSSPVTKSFNRGEC

### 3D: Additional Bispecific Antibody Formats

### 3D(1): 1+1 Common Light Chain (CLC) Format

Another heterodimeric antibody format that finds particular use in subject anti-TL1A×anti-IL23 antibodies provided herein is the 1+1 Common Light Chain (CLC) format (depicted schematically in FIG. 1, Panel C) which comprises a first monomer comprising VH1-CH1-hinge-CH2-CH3, a second monomer comprising VH2-CH1-hinge-CH2-CH3, and a third monomer comprising VL-CL. The VL pairs with the VH1 to form a binding domain with a first antigen binding specificity; and the VL pairs with the VH2 to form a binding domain with a second antigen binding specificity. Sequences for illustrative TL1A×IL23 bsAbs in the 1+1 CLC format are depicted in Table 33. CDRs are underlined and slashes indicate the border(s) between the variable regions, linkers, Fc regions, and constant domains. It should be noted that the TL1A×IL23 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S or any other half-life extension variants and/or any other ablation variant (as depicted in Table 1) in one or preferably both Fc domains Additionally, each sequence can utilize any other heterodimeric Fc variant as depicted in Tables 12-17.

TABLE 33

| | SEQ ID NO \| Antigen | | | | | |
|---|---|---|---|---|---|---|
| Xencor ID | Chain 1 | | Chain 2 | | Chain 3 | |
| XENP51035 | 1156 | IL23 | 1157 | TL1A | 1158 | IL23 |
| XENP51036 | 1159 | TL1A | 1160 | IL23 | 1161 | IL23 |

### 3D(2): 2+1 Fab₂-scFv-Fc

Another exemplary format utilizing Fab domains and scFv is the 2+1 Fab-scFv-Fc format (depicted schematically in FIG. 1, Panel B) which comprises a first monomer comprising a VH domain covalently attached to a single-chain Fv (“scFv”) with a first antigen binding specificity covalently attached to a first heterodimeric Fc domain i.e., VH-CH1-domain-linker-scFv-domain linker-CH2-CH3, a second monomer comprising a heavy chain i.e., VH-CH1-hinge-CH2-CH3, wherein the CH2-CH3 is a second heterodimeric Fc domain complementary to the first heterodimeric Fc domain, and a light chain (LC) transfected separately so that a Fab domain having a second antigen binding specificity is formed with the variable heavy domain.

The amino acid sequence for an illustrative TL1A×IL23 bsAb in the 2+2 Fab₂-scFv-Fc format is depicted in Table 34 as XENP50965. Table 34 depicts the sequences for illustrative TL1A×IL23 bsAbs in additional bispecific formats. CDRs are underlined and slashes indicate the border(s) between the variable regions, linkers, Fc regions, and constant domains. It should be noted that the TL1A×IL23 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S or any other half-life extension variants and/or any other ablation variant (as depicted in Table 1) in one or preferably both Fe domains Additionally, each sequence can utilize any other heterodimeric Fc variant as depicted in Tables 12-17.

TABLE 34

| | SEQ ID NO \| Antigen | | | | | |
|---|---|---|---|---|---|---|
| Xencor ID | Chain 1 | | Chain 2 | | Chain 3 | |
| XENP50966 | 1162 | IL23 | 1163 | IL23 | 1164 | IL23 |
| XENP50965 | 1165 | IL23 | 1166 | TL1A | 1167 | IL23 |
| XENP50963 | 1168 | IL23 | 1169 | IL23 | | |
| XENP50964 | 1170 | TL1A | 1171 | TL1A | | |
| XENP50961 | 1172 | IL23 | 1173 | IL23 | | |
| XENP50962 | 1174 | TL1A | 1175 | TL1A | | |

3D(3): 2+1 mAb-scFv

Another exemplary format utilizing Fab domains and scFv is the 2+1 mAb-scFv format (depicted schematically in FIG. 1, Panel E) which comprises a first monomer comprising a heavy chain covalently attached to a single-chain Fv (“scFv”) with a first antigen binding specificity covalently i.e., VH-CH1-hinge-CH2-CH3-domain linker-scFv, a second monomer comprising a heavy chain i.e., VH-CH1-hinge-CH2-CH3, wherein the CH2-CH3 is a second heterodimeric Fc domain complementary to the first heterodimeric Fc domain, and a light chain (LC) transfected separately so that a Fab domain having a second antigen binding specificity is formed with the variable heavy domain.

The amino acid sequence for an illustrative TL1A×IL23 bsAb in the 2+1 mAb-scFv format is depicted in Table 34 as XENP50966.

3D(4): 2+2 Fab₂-scFv₂-Fc

Another heterodimeric antibody format antibody format that finds particular use in subject anti-TL1A×anti-IL23 antibodies provided herein is the 2+2 Fab₂-scFv₂-Fc format illustrated in FIG. 1, Panel R. This antibody format includes four antigen binding domains: two Fab portions, and two scFv portions that are each attached between the Fab domains and the Fc domains.

In this embodiment, two identical first monomers each comprise, from N- to C-terminal, VH1-CH1-scFv-domain linker-CH2-CH3, where the scFv domains each comprise a second VH (VH2), a second VL (VL2) and a scFv linker. In embodiments, this format includes two identical light chains (VL1-CL). In this format, the VH1s are each a first variable heavy (VH) domain, the VL1s are each a first variable light (VL) domain, the VH2 is a second variable heavy domain, and the VL2 is a second variable light domain. In some embodiments, the format includes two identical common light chains (each VL1-CL), wherein each of the common light chains associates with one of the VH1-CH1 of the first and second monomers to form two identical Fabs (i.e., first antigen binding domains). In some embodiments, the scFv is the second antigen binding domain.

The amino acid sequence for an illustrative TL1A×IL23 bsAb in the 2+1 Fab2-scFv₂-Fc format is depicted in Table 34 as XENP50961 and XENP50962.

3D(5): 2+2 mAb-scFv

Another heterodimeric antibody format antibody format that finds particular use in subject anti-TL1A×anti-IL23 antibodies provided herein is the 2+2 mAb-scFv format illustrated in FIG. 1, Panel O. This antibody format includes four antigen binding domains: two Fab portions, and two scFv portions that are each attached to the C-terminal of one of the heavy chains.

In this embodiment, two identical first monomers each comprise, from N- to C-terminal, VH1-CH1-hinge-CH2-CH3-domain linker-scFv domain, where the scFv domains each comprise a second VH (VH2), a second VL (VL2) and a scFv linker. As for all the scFv domains herein, the scFv domain can be in either orientation, from N- to C-terminal, VH2-scFv linker-VL2 or VL2-scFv linker-VH2. Accordingly, the first monomers may comprise, from N- to C-terminal, VH1-CH1-hinge-CH2-CH3-domain linker-VH2-scFv linker-VL2 or VH1-CH1-hinge-CH2-CH3-domain linker-VL2-scFv linker-VH2. The composition also comprises a light chain, VL1-CL. In embodiments, this format includes two identical light chains (VL1-CL). In this format, the VH1s are each a first variable heavy (VH) domain, the VL1s are each a first variable light (VL) domain, the VH2 is a second variable heavy domain, and the VL2 is a second variable light domain. In some embodiments, the format includes two identical common light chains (each VL1-CL), wherein each of the common light chains associates with one of the VH1-CH1 of the first and second monomers to form two identical Fabs (i.e., first antigen binding domains). In some embodiments, the scFv is the second antigen binding domain.

The amino acid sequence for an illustrative TL1A×IL23 bsAb in the 2+2 mAb-scFv format is depicted in Table 34 as XENP50963 and XENP50964.

In some embodiments, the Fc domains of the additional bispecific antibody formats are variant Fc domains that include ablation variants (including those shown in Table 1). In some embodiments, each of the first and second variant Fc domains include ablation variants E233P/L234V/L235A/G236_/S267K, wherein numbering is according to EU numbering.

In some embodiments for additional bispecific antibody formats that include scFv, the scFv may optionally include a charged scFv linker (including those shown in Table 4). Optionally, any of the scFvs can include G44C and A100C for improving stability. As for all the scFv domains herein, the scFv domain can be in either orientation, from N- to C-terminal, VH-scFv linker-VL or VL-scFv linker-VH.

In some embodiments, the additional bispecific antibody formats provided herein includes half-life extending variants as is known in the art. In some embodiments, the half-life extending variants are M428L/N434S, wherein numbering is according to EU numbering.

### Example 4: TL1A×IL23p19 Bispecifics are Functionally Active on Both TL1A and IL23 Axes

In order to test the hypothesis that TL1A×IL23 bispecific antibodies are able to inhibit both TL1A and IL23 signaling, initial bispecific antibodies were engineered with the TL1A and IL23 binding domains described in Examples 1A and 2B.

To evaluate the inhibitory activity of TL1A×IL-23 bispecific antibodies on the IL-23 signaling axis, an IL-23 reporter bioassay (Promega, Madison, WI) was performed. This assay employs a genetically engineered human cell line that co-expresses the IL-23 receptor and a luciferase reporter gene under the control of an IL-23-responsive element. Upon IL-23 binding, receptor-mediated signaling activates the response element, inducing luciferase expression and generating a bioluminescent signal proportional to IL-23 activity. For the assay, TL1A×IL-23 bispecifics were serially diluted and mixed with IL-23 (final concentration: 10 ng/mL in R10 medium) and incubated at 37° C. for 75 minutes. Reporter cells were then added and further incubated for 6 hours. Following incubation, Bio-Glo™ reagent (Promega) was added, and luminescence was measured using a PerkinElmer EnVision plate reader. As depicted in FIG. 2, the TL1A×IL23 bsAbs in both the OrthoFab and the 1+1 Fab-scFv-Fc format showed efficacy in inhibiting IL23 activity, albeit less potently than the bivalent IL23 mAb utilizing the same TL23 binding domain.

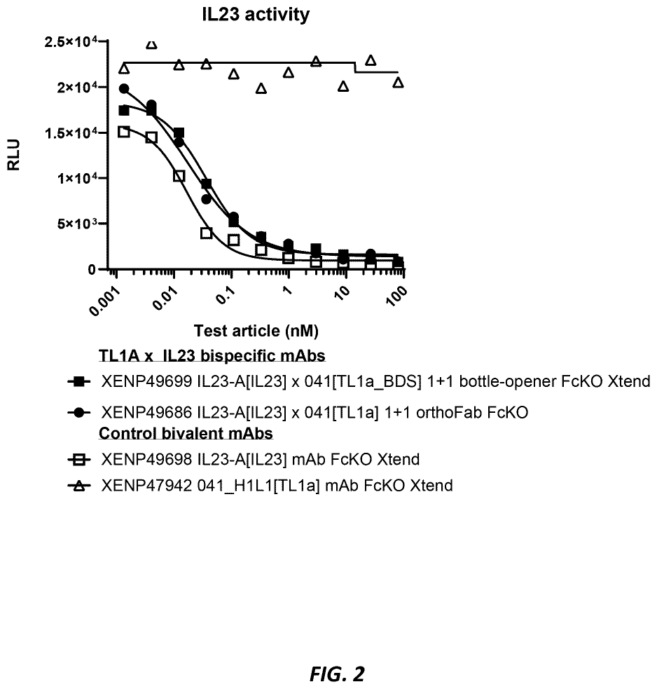

To assess the activity of TL1A×IL-23 bispecific antibodies on the TL1A axis, a TL1A-responsive luciferase reporter Jurkat cell line (BPS Bioscience, San Diego, CA) was used. These cells stably express a luciferase reporter under the control of NFκB response elements, enabling quantification of TL1A-mediated signaling through bioluminescence. Briefly in the standard assay format, 5×10⁵ reporter cells in R10 medium were seeded into plates and cultured overnight. The following day, cells were resuspended and seeded at 30,000 cells per well, then incubated with TL1A×IL-23 bispecific test articles across a serial dilution range (120 nM to 0.002 nM). Plates were incubated at 37° C. for 5 hours. After incubation, Bio-Glo™ reagent (Promega) was added, and luminescence was measured using a PerkinElmer EnVision plate reader. As depicted in FIG. 3, TL1A×IL23 bsAbs in both the OrthoFab and the 1+1 Fab-scFv-Fc format showed efficacy in inhibiting TL1A activity comparable to the bivalent TL1A mAb utilizing the same TL1A binding domain.

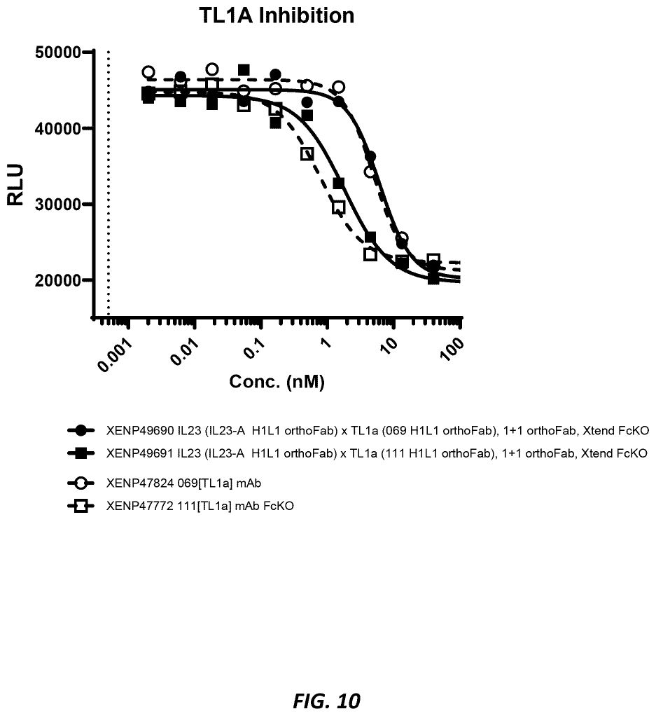

In another set of experiments, TL1A×TL23 bispecifics utilizing anti-TL1A clone 069 or clone 111 were investigated. As depicted in FIG. 10, these bispecific antibodies were also efficacious in inhibiting TL1A activity. While additional anti-TL1A binding domains were suitable, the 041 clone was selected for further development as it demonstrated superior pharmacokinetics in bivalent monospecific mAb format (Table 35). Table 35 depicts serum concentration of bivalent TL1A mAbs based on Clone 041, Clone 069, or Clone 111 in cynomolgus monkeys dosed at 5 mg/kg.

TABLE 35

| TL1A Clone | t_{1/2} (day) | Cₘₐₓ (μg/mL) | AUCₗₐₛₜ (day*μg/mL) |
|---|---|---|---|
| 111 | 21.8 | 184 | 1380 |
| 041 | 29.5 | 213 | 1560 |
| 069 | 18.8 | 183 | 1170 |

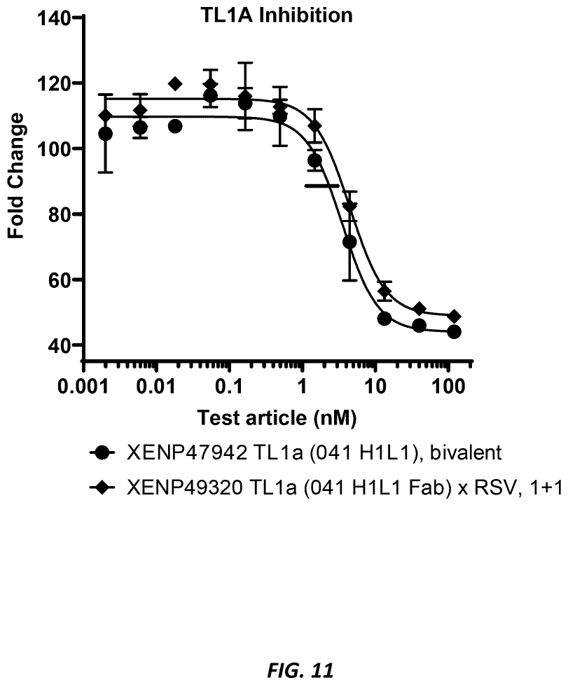

To deconvolute the monovalent activities of the TL1A and IL23 binding domains, a bispecific antibody was generated pairing the TL1A binding domain with an RSV binding domain. As depicted in FIG. 11, XENP49320 having monovalent binding for TL1A via the 041 binding domain was less efficacious than corresponding bivalent TL1A mAb.

### Example 5: Engineering Affinity Enhanced TL1A and IL23p19 Binding Domains

As per Example 4, monovalent stoichiometry of the TL1A clone 041 and IL23 clone IL23-A in the bispecific antibodies reduces the effective potency of the bispecific molecule due to the reduced number of binding sites relative to the bivalent counterparts. This was further investigated via mechanism-based quantitative systems pharmacology (QSP) model to fit the drug pharmacokinetic and pharmacodynamic data along with in-vivo binding. The QSP model was used to simulate the anticipated PK profile of the intended bispecific owing to target mediated drug deposition (TMDD) from the anti-TL1A Fab, the extended antibody half-life due to mutations in the antibody Fc region to improve binding to FcRn, as well as to predict the expected binding to TL1A or IL23 with bispecific antibody at a range of dose levels and regiments. Based on these simulations, approximately 3-fold improvement in Fab binding affinity towards both TL1A and TL23 was predicted to achieve similar level of target engagement at the comparator bivalent antibodies.

Accordingly, TL1A clone 041 variants described in Example 1B and IL23 clone IL23-A variants described in Example 2A were generated. To identify favorites, initial variants from either the output of phage display for TL1A clone 041 or rational design for IL23 clone TL23-A were recombinantly made, and investigated for their binding via Octet, a BioLayer Interferometry (BLI)-based method, or Biacore, surface plasmon resonance (SPR)-based method. Experimental steps for Biacore generally included the following: Immobilization (capture of ligand onto a sensor chip); Association (flowing of various concentrations of analyte over sensor chip); and Dissociation (flowing buffer over the sensor chips) in order to determine the affinity of the test articles. Experimental steps for Octet generally include the following: Immobilization (capture of ligand to a biosensor); Association (dipping of ligand-coated biosensors into wells containing serial dilutions of the analyte); and Dissociation (returning of biosensors to well containing buffer) in order to determine the affinity of the test articles.

For initial screening, only a single concentration of analyte was used, and dissociation rate (k_{off}) ranking was used to pick a subset of VH and VL variants to move forward. These subsets of variants were engineered in the relevant bispecific antibody formats, and more accurate K_{D} values were determined. The favorite VH and VL variants were then combined as VH/VL pairs and engineered in the relevant bispecific antibody formats for further assessment. In the case of 041, favorite VH/VL affinity variants were further paired with superhumanization substitutions that did not impact affinity. Affinities for exemplary variants are depicted in Tables 36-40. Table 36 depicts Octet screening of illustrative 041 variants (produced as His-tagged Fabs). Table 37 depicts binding affinity of illustrative 041 variants combined with superhumanization variants (produced as 1+1 Fab-scFv-Fc bispecific antibodies) for TL1A at 37° C. Table 38 depicts Biacore screening of illustrative IL23-A variants (produced as His-tagged Fabs) that were shortlisted based on off rate ranking. Table 39 depicts binding affinity of illustrative IL23-A variants (produced as 1+1 Fab-scFv-Fc bispecific antibodies) for IL23 at 37° C. Table 40 depicts binding affinity of illustrative IL23-A variants (produced as 1+1 Fab-scFv-Fc bispecific antibodies) for IL23 at 37° C. In particular as depicted in FIG. 8 for exemplary 041 and IL23-A affinity-engineered variants, dissociation rate was significantly improved over the parental clone.

TABLE 36

| | K_{D} (M) | kₐ (1/Ms) | k_{d} (1/s) |
|---|---|---|---|
| TL1a (041 H1L1), Fab-His | 1.17E−10 | 2.53E+06 | 2.96E−04 |
| | 1.20E−10 | 2.39E+06 | 2.87E−04 |
| 041[TL1A]_H1.95_{—} | 1.64E−11 | 7.14E+06 | 1.17E−04 |
| L1.76_Fab_His | 1.71E−11 | 6.59E+06 | 1.13E−04 |
| 041[TL1A]_H1.127_L1_Fab_His | 3.77E−11 | 4.80E+06 | 1.81E−04 |
| | 3.84E−11 | 4.59E+06 | 1.76E−04 |
| 041[TL1A]_H1.128_L1_Fab_His | 2.21E−11 | 4.44E+06 | 9.79E−05 |
| | 2.27E−11 | 4.15E+06 | 9.43E−05 |
| 041[TL1A]_H1.150_L1_Fab_His | 1.58E−10 | 1.05E+06 | 1.67E−04 |
| | 1.59E−10 | 1.02E+06 | 1.61E−04 |
| 041[TL1A]_H1.156_L1_Fab_His | 1.40E−10 | 2.10E+06 | 2.93E−04 |
| | 1.37E−10 | 2.04E+06 | 2.78E−04 |

TABLE 37

| XENP | Short Name | K_{D} (M) | kₐ (1Ms) | k_{d} (1/s) |
|---|---|---|---|---|
| 49335 | TL1a (041 H1L1 Fab) × IL23 (IL23-A H1L1 scFv) | 1.90E−10 | 1.08E+06 | 2.05E−04 |
| 49335 | TL1a (041 H1L1 Fab) × IL23 (IL23-A H1L1 scFv) | 1.79E−10 | 1.16E+06 | 2.07E−04 |
| 52371 | 041[TL1A]_H1_L1.25_Fab × IL23-A[IL23]_H1L1 | 1.92E−10 | 1.05E+06 | 2.01E−04 |
| 52371 | 041[TL1A]_H1_L1.25_Fab × IL23-A[IL23]_H1L1 | 2.03E−10 | 9.93E+05 | 2.02E−04 |
| 52372 | 041[TL1A]_H1_L1.26_Fab × IL23-A[IL23]_H1L1 | 1.83E−10 | 1.02E+06 | 1.86E−04 |
| 52372 | 041[TL1A]_H1_L1.26_Fab × IL23-A[IL23]_H1L1 | 1.66E−10 | 1.11E+06 | 1.85E−04 |
| 52373 | 041[TL1A]_H1_L1.27_Fab × IL23-A[IL23]_H1L1 | 1.95E−10 | 1.05E+06 | 2.04E−04 |
| 52373 | 041[TL1A]_H1_L1.27_Fab × IL23-A[IL23]_H1L1 | 1.85E−10 | 1.11E+06 | 2.05E−04 |
| 52374 | 041[TL1A]_H1.95_L1_Fab × IL23-A[IL23]_H1L1 | 4.19E−11 | 1.95E+06 | 8.18E−05 |
| 52374 | 041[TL1A]_H1.95_L1_Fab × IL23-A[IL23]_H1L1 | 3.73E−11 | 2.19E+06 | 8.18E−05 |
| 52382 | 041[TL1A]_H1.127_L1_Fab × IL23-A[IL23]_H1L1 | 9.13E−11 | 1.72E+06 | 1.57E−04 |
| 52382 | 041[TL1A]_H1.127_L1_Fab × IL23-A[IL23]_H1L1 | 9.80E−11 | 1.58E+06 | 1.55E−04 |
| 52383 | 041[TL1A]_H1.127_L1.25_Fab × IL23-A[IL23]_H1L1 | 9.88E−11 | 1.54E+06 | 1.53E−04 |
| 52383 | 041[TL1A]_H1.127_L1.25_Fab × IL23-A[IL23]_H1L1 | 1.02E−10 | 1.55E+06 | 1.58E−04 |
| 52384 | 041[TL1A]_H1.127_L1.26_Fab × IL23-A[IL23]_H1L1 | 9.85E−11 | 1.39E+06 | 1.37E−04 |
| 52384 | 041[TL1A]_H1.127_L1.26_Fab × IL23-A[IL23]_H1L1 | 9.20E−11 | 1.50E+06 | 1.38E−04 |
| 52385 | 041[TL1A]_H1.127_L1.27_Fab × IL23-A[IL23]_H1L1 | 8.20E−11 | 1.87E+06 | 1.53E−04 |
| 52385 | 041[TL1A]_H1.127_L1.27_Fab × IL23-A[IL23]_H1L1 | 7.76E−11 | 2.07E+06 | 1.61E−04 |
| 52386 | 041[TL1A]_H1.128_L1_Fab × IL23-A[IL23]_H1L1 | 8.13E−11 | 1.63E+06 | 1.32E−04 |
| 52386 | 041[TL1A]_H1.128_L1_Fab × IL23-A[IL23]_H1L1 | 6.33E−11 | 2.21E+06 | 1.40E−04 |
| 52387 | 041[TL1A]_H1.128_L1.25_Fab × IL23-A[IL23]_H1L1 | 9.81E−11 | 1.31E+06 | 1.28E−04 |
| 52387 | 041[TL1A]_H1.128_L1.25_Fab × IL23-A[IL23]_H1L1 | 1.01E−10 | 1.37E+06 | 1.38E−04 |
| 52388 | 041[TL1A]_H1.128_L1.26_Fab × IL23-A[IL23]_H1L1 | 1.17E−10 | 1.24E+06 | 1.44E−04 |
| 52388 | 041[TL1A]_H1.128_L1.26_Fab × IL23-A[IL23]_H1L1 | 1.17E−10 | 1.26E+06 | 1.47E−04 |
| 52389 | 041[TL1A]_H1.128_L1.27_Fab × IL23-A[IL23]_H1L1 | 8.70E−11 | 1.47E+06 | 1.28E−04 |
| 52389 | 041[TL1A]_H1.128_L1.27_Fab × IL23-A[IL23]_H1L1 | 8.20E−11 | 1.55E+06 | 1.27E−04 |
| 52390 | 041[TL1A]_H1.132_L1_Fab × IL23-A[IL23]_H1L1 | 9.68E−11 | 1.22E+06 | 1.18E−04 |
| 52390 | 041[TL1A]_H1.132_L1_Fab × IL23-A[IL23]_H1L1 | 5.86E−11 | 2.14E+06 | 1.26E−04 |
| 52391 | 041[TL1A]_H1.132_L1.25_Fab × IL23-A[IL23]_H1L1 | 9.30E−11 | 1.23E+06 | 1.14E−04 |
| 52391 | 041[TL1A]_H1.132_L1.25_Fab × IL23-A[IL23]_H1L1 | 9.28E−11 | 1.31E+06 | 1.21E−04 |
| 52392 | 041[TL1A]_H1.132_L1.26_Fab × IL23-A[IL23]_H1L1 | 1.18E−10 | 1.09E+06 | 1.29E−04 |
| 52392 | 041[TL1A]_H1.132_L1.26_Fab × IL23-A[IL23]_H1L1 | 1.08E−10 | 1.15E+06 | 1.24E−04 |
| 52393 | 041[TL1A]_H1.132_L1.27_Fab × IL23-A[IL23]_H1L1 | 1.12E−10 | 1.09E+06 | 1.23E−04 |
| 52393 | 041[TL1A]_H1.132_L1.27_Fab × IL23-A[IL23]_H1L1 | 9.90E−11 | 1.19E+06 | 1.18E−04 |
| 52394 | 041[TL1A]_H1.95_L1.76_Fab × IL23-A[IL23]_H1L1 | 6.91E−11 | 1.72E+06 | 1.19E−04 |
| 52394 | 041[TL1A]_H1.95_L1.76_Fab × IL23-A[IL23]_H1L1 | 7.00E−11 | 1.75E+06 | 1.23E−04 |
| 52395 | 041[TL1A]_H1.115_L1.95_Fab × IL23-A[IL23]_H1L1 | 2.48E−10 | 1.29E+06 | 3.20E−04 |
| 52395 | 041[TL1A]_H1.115_L1.95_Fab × IL23-A[IL23]_H1L1 | 2.57E−10 | 1.31E+06 | 3.36E−04 |
| 52396 | 041[TL1A]_H1_L1.76_Fab × IL23-A[IL23]_H1L1 | 5.85E−10 | 8.37E+05 | 4.90E−04 |
| 52396 | 041[TL1A]_H1_L1.76_Fab × IL23-A[IL23]_H1L1 | 5.87E−10 | 8.80E+05 | 5.17E−04 |

TABLE 38

| | K_{D} (M) | kₐ (1/Ms) | k_{d} (1/s) |
|---|---|---|---|
| IL23-A[IL23]_H1L1_Fab | 1.79E−11 | 2.49E+06 | 4.47E−05 |
| IL23-A[IL23]_H1.13_L1_Fab | 2.00E−11 | 3.02E+06 | 6.05E−05 |
| IL23-A[IL23]_H1.25_L1_Fab | 1.81E−11 | 3.15E+06 | 5.71E−05 |
| IL23-A[IL23]_H1.26_L1_Fab | 1.44E−11 | 4.21E+06 | 6.06E−05 |
| IL23-A[IL23]_H1.27_L1_Fab | 1.54E−11 | 3.83E+06 | 5.91E−05 |
| IL23-A[IL23]_H1.28_L1_Fab | 1.40E−11 | 4.08E+06 | 5.70E−05 |
| IL23-A[IL23]_H1.37_L1_Fab | 3.65E−11 | 3.30E+06 | 1.20E−04 |
| IL23-A[IL23]_H1.41_L1_Fab | 2.14E−11 | 2.02E+06 | 4.33E−05 |
| IL23-A[IL23]_H1.55_L1_Fab | 2.36E−11 | 1.71E+06 | 4.03E−05 |
| IL23-A[IL23]_H1.82_L1_Fab | 2.11E−11 | 2.46E+06 | 5.20E−05 |
| IL23-A[IL23]_H1.88_L1_Fab | 2.07E−11 | 2.49E+06 | 5.16E−05 |
| IL23-A[IL23]_H1.89_L1_Fab | 1.82E−11 | 2.99E+06 | 5.43E−05 |
| IL23-A[IL23]_H1.90_L1_Fab | 1.77E−11 | 2.76E+06 | 4.89E−05 |
| IL23-A[IL23]_H1.91_L1_Fab | 2.10E−11 | 3.09E+06 | 6.50E−05 |
| IL23-A[IL23]_H1.92_L1_Fab | 1.76E−11 | 2.59E+06 | 4.57E−05 |
| IL23-A[IL23]_H1.93_L1_Fab | 1.93E−11 | 3.10E+06 | 5.98E−05 |
| IL23-A[IL23]_H1.101_L1_Fab | 1.68E−11 | 2.66E+06 | 4.47E−05 |
| IL23-A[IL23]_H1.102_L1_Fab | 4.42E−11 | 1.26E+06 | 5.55E−05 |
| IL23-A[IL23]_H1.104_L1_Fab | 2.15E−11 | 2.66E+06 | 5.72E−05 |
| IL23-A[IL23]_H1.113_L1_Fab | 2.60E−11 | 1.96E+06 | 5.11E−05 |
| IL23-A[IL23]_H1.114_L1_Fab | 1.91E−11 | 2.61E+06 | 4.96E−05 |
| IL23-A[IL23]_H1.120_L1_Fab | 2.28E−11 | 2.55E+06 | 5.82E−05 |
| IL23-A[IL23]_H1.132_L1_Fab | 2.38E−11 | 2.72E+06 | 6.48E−05 |
| IL23-A[IL23]_H1.137_L1_Fab | 4.40E−11 | 1.61E+06 | 7.08E−05 |
| IL23-A[IL23]_H1.138_L1_Fab | 2.60E−11 | 2.26E+06 | 5.89E−05 |
| IL23-A[IL23]_H1.140_L1_Fab | 2.59E−11 | 1.89E+06 | 4.89E−05 |
| IL23-A[IL23]_H1.141_L1_Fab | 3.49E−11 | 1.16E+06 | 4.06E−05 |
| IL23-A[IL23]_H1.142_L1_Fab | 3.84E−11 | 1.19E+06 | 4.56E−05 |
| IL23-A[IL23]_H1.143_L1_Fab | 8.05E−11 | 2.42E+06 | 1.95E−04 |
| IL23-A[IL23]_H1.147_L1_Fab | 2.79E−11 | 2.96E+06 | 8.25E−05 |
| IL23-A[IL23]_H1.149_L1_Fab | 3.99E−11 | 3.00E+06 | 1.20E−04 |
| IL23-A[IL23]_H1.156_L1_Fab | 1.73E−09 | 2.15E+05 | 3.71E−04 |
| IL23-A[IL23]_H1.168_L1_Fab | 9.09E−11 | 2.58E+06 | 2.35E−04 |
| IL23-A[IL23]_H1.169_L1_Fab | 2.24E−11 | 6.64E+06 | 1.48E−04 |
| IL23-A[IL23]_H1.182_L1_Fab | 4.73E−10 | 4.76E+05 | 2.25E−04 |
| IL23-A[IL23]_H1.187_L1_Fab | 2.75E−11 | 1.76E+06 | 4.83E−05 |
| IL23-A[IL23]_H1.188_L1_Fab | 3.68E−11 | 1.83E+06 | 6.73E−05 |
| IL23-A[IL23]_H1.189_L1_Fab | 7.68E−11 | 2.39E+06 | 1.84E−04 |
| IL23-A[IL23]_H1.190_L1_Fab | 5.48E−11 | 3.18E+06 | 1.75E−04 |
| IL23-A[IL23]_H1.191_L1_Fab | 2.56E−10 | 1.97E+06 | 5.05E−04 |
| IL23-A[IL23]_H1.192_L1_Fab | 1.50E−10 | 8.10E+05 | 1.21E−04 |
| IL23-A[IL23]_H1_L1.50_Fab | 2.56E−11 | 2.45E+06 | 6.28E−05 |
| IL23-A[IL23]_H1_L1.54_Fab | 1.56E−10 | 3.69E+05 | 5.76E−05 |
| IL23-A[IL23]_H1_L1.58_Fab | 1.50E−10 | 3.38E+05 | 5.06E−05 |
| IL23-A[IL23]_H1_L1.65_Fab | 2.14E−11 | 2.09E+06 | 4.48E−05 |
| IL23-A[IL23]_H1_L1.69_Fab | 2.02E−11 | 2.02E+06 | 4.08E−05 |

TABLE 39

| XENP | | K_{D} (M) | kₐ (1/Ms) | k_{d} (1/s) |
|---|---|---|---|---|
| 49699 | IL23-A[IL23]_H1L1_Fab- | 1.36E−10 | 6.37E+05 | 8.68E−05 |
| | 041[TL1a]_H1_G44C_L1_A100C | | | |
| 52850 | IL23-A[IL23]_H2L1_Fab- | 1.37E−10 | 5.63E+05 | 7.70E−05 |
| | 041[TL1a]_H1_G44C_L1_A100C | | | |
| 53110 | IL23-A[IL23]_H1_L1.71_Fab- | 1.13E−10 | 7.43E+05 | 8.40E−05 |
| | 041[TL1a]_H1_G44C_L1_A100C | | | |
| 53111 | IL23-A[IL23]_H1_L1.69_Fab- | 6.68E−11 | 9.58E+05 | 6.40E−05 |
| | 041[TL1a]_H1_G44C_L1_A100C | | | |
| 53112 | IL23-A[IL23]_H1_L1.58_Fab- | 1.56E−10 | 5.79E+05 | 9.02E−05 |
| | 041[TL1a]_H1_G44C_L1_A100C | | | |
| 53113 | IL23-A[IL23]_H1_L1.79_Fab- | 7.88E−11 | 6.61E+05 | 5.21E−05 |
| | 041[TL1a]_H1_G44C_L1_A100C | | | |
| 53114 | IL23-A[IL23]_H1_L1.42_Fab- | 1.27E−10 | 5.94E+05 | 7.57E−05 |
| | 041[TL1a]_H1_G44C_L1_A100C | | | |
| 53115 | IL23-A[IL23]_H2_L1.71_Fab- | 1.19E−10 | 5.49E+05 | 6.55E−05 |
| | 041[TL1a]_H1_G44C_L1_A100C | | | |
| 53116 | IL23-A[IL23]_H2_L1.69_Fab- | 6.71E−11 | 7.46E+05 | 5.01E−05 |
| | 041[TL1a]_H1_G44C_L1_A100C | | | |
| 53117 | IL23-A[IL23]_H2_L1.58_Fab- | 1.49E−10 | 4.69E+05 | 7.00E−05 |
| | 041[TL1a]_H1_G44C_L1_A100C | | | |
| 53118 | IL23-A[IL23]_H2_L1.79_Fab- | 8.25E−11 | 5.86E+05 | 4.83E−05 |
| | 041[TL1a]_H1_G44C_L1_A100C | | | |
| 53119 | IL23-A[IL23]_H2_L1.42_Fab- | 1.38E−10 | 4.88E+05 | 6.73E−05 |
| | 041[TL1a]_H1_G44C_L1_A100C | | | |

TABLE 40

| XENP | | K_{D} (M) | kₐ (1/Ms) | k_{d} (1/s) |
|---|---|---|---|---|
| 49699 | IL23-A[IL23]_H1L1_Fab- | 9.90E−11 | 6.58E+05 | 6.51E−05 |
| | 041[TL1a]_H1_G44C_L1_A100C | | | |
| 53194 | IL23-A[IL23]_H1_L1.114_Fab- | 5.88E−11 | 8.09E+05 | 4.76E−05 |
| | 041[TL1a]_H1_G44C_L1_A100C | | | |
| 53195 | IL23-A[IL23]_H1_L1.115_Fab- | 1.97E−11 | 9.45E+05 | 1.86E−05 |
| | 041[TL1a]_H1_G44C_L1_A100C | | | |
| 53196 | IL23-A[IL23]_H1_L1.116_Fab- | 3.25E−11 | 9.14E+05 | 2.97E−05 |
| | 041[TL1a]_H1_G44C_L1_A100C | | | |
| 53197 | IL23-A[IL23]_H1.41_L1_Fab- | 1.12E−10 | 5.49E+05 | 6.16E−05 |
| | 041[TL1a]_H1_G44C_L1_A100C | | | |
| 53198 | IL23-A[IL23]_H1.55_L1_Fab- | 8.67E−11 | 7.68E+05 | 6.66E−05 |
| | 041[TL1a]_H1_G44C_L1_A100C | | | |
| 53199 | IL23-A[IL23]_H1.94_L1_Fab- | 8.57E−11 | 8.26E+05 | 7.07E−05 |
| | 041[TL1a]_H1_G44C_L1_A100C | | | |
| 53200 | IL23-A[IL23]_H1.101_L1_Fab- | 8.43E−11 | 8.35E+05 | 7.04E−05 |
| | 041[TL1a]_H1_G44C_L1_A100C | | | |
| 53201 | IL23-A[IL23]_H1.113_L1_Fab- | 8.60E−11 | 1.05E+06 | 9.00E−05 |
| | 041[TL1a]_H1_G44C_L1_A100C | | | |
| 53202 | IL23-A[IL23]_H1.125_L1_Fab- | 1.02E−10 | 8.13E+05 | 8.30E−05 |
| | 041[TL1a]_H1_G44C_L1_A100C | | | |
| 53203 | IL23-A[IL23]_H1.140_L1_Fab- | 1.71E−10 | 4.81E+05 | 8.24E−05 |
| | 041[TL1a]_H1_G44C_L1_A100C | | | |
| 53204 | IL23-A[IL23]_H1.141_L1_Fab- | 1.44E−10 | 5.64E+05 | 8.15E−05 |
| | 041[TL1a]_H1_G44C_L1_A100C | | | |
| 53205 | IL23-A[IL23]_H1.142_L1_Fab- | 1.66E−10 | 5.43E+05 | 8.99E−05 |
| | 041[TL1a]_H1_G44C_L1_A100C | | | |
| 53206 | IL23-A[IL23]_H1.169_L1_Fab- | 1.57E−10 | 4.57E+05 | 7.16E−05 |
| | 041[TL1a]_H1_G44C_L1_A100C | | | |
| 53207 | IL23-A[IL23]_H1.187_L1_Fab- | 1.08E−10 | 8.01E+05 | 8.68E−05 |
| | 041[TL1a]_H1_G44C_L1_A100C | | | |
| 53208 | IL23-A[IL23]_H1.195_L1_Fab- | 5.61E−10 | 8.83E+04 | 4.96E−05 |
| | 041[TL1a]_H1_G44C_L1_A100C | | | |
| 53209 | IL23-A[IL23]_H1.195_L1.116_Fab- | 2.19E−10 | 2.86E+05 | 6.26E−05 |
| | 041 [TL1a]_H1_G44C_L1_A100C | | | |
| 53210 | IL23-A[IL23]_H1.94_L1.116_Fab- | 1.24E−11 | 1.11E+06 | 1.38E−05 |
| | 041[TL1a]_H1_G44C_L1_A100C | | | |
| 53211 | IL23-A[IL23]_H1.187_L1.116_Fab- | 3.01E−11 | 1.22E+06 | 3.67E−05 |
| | 041[TL1a]_H1_G44C_L1_A100C | | | |
| 53212 | IL23-A[IL23]_H1.169_L1.116_Fab- | 4.23E−11 | 8.34E+05 | 3.52E−05 |
| | 041 [TL1a]_H1_G44C_L1_A100C | | | |
| 53213 | IL23-A[IL23]_H1.101_L1.116_Fab- | 2.37E−11 | 1.30E+06 | 3.08E−05 |
| | 041[TL1a]_H1_G44C_L1_A100C | | | |
| 53214 | IL23-A[IL23]_H1.94_L1.69_Fab- | 2.95E−11 | 1.34E+06 | 3.94E−05 |
| | 041[TL1a]_H1_G44C_L1_A100C | | | |
| 53215 | IL23-A[IL23]_H1.94_L1.58_Fab- | 1.19E−10 | 4.90E+05 | 5.84E−05 |
| | 041[TL1a]_H1_G44C_L1_A100C | | | |
| 53216 | IL23-A[IL23]_H1.94_L1.79_Fab- | 6.38E−11 | 5.56E+05 | 3.55E−05 |
| | 041[TL1a]_H1_G44C_L1_A100C | | | |
| 53217 | IL23-A[IL23]_H1.101_L1.69_Fab- | 6.41E−11 | 7.33E+05 | 4.70E−05 |
| | 041[TL1a]_H1_G44C_L1_A100C | | | |
| 53218 | IL23-A[IL23]_H1.101_L1.58_Fab- | 1.57E−10 | 4.77E+05 | 7.50E−05 |
| | 041[TL1a]_H1_G44C_L1_A100C | | | |
| 53219 | IL23-A[IL23]_H1.101_L1.79_Fab- | 9.11E−11 | 5.11E+05 | 4.65E−05 |
| | 041[TL1a]_H1_G44C_L1_A100C | | | |
| 53220 | IL23-A[IL23]_H1.169_L1.69_Fab- | 4.66E−11 | 8.39E+05 | 3.91E−05 |
| | 041[TL1a]_H1_G44C_L1_A100C | | | |
| 53221 | IL23-A[IL23]_H1.169_L1.58_Fab- | 9.89E−11 | 5.37E+05 | 5.31E−05 |
| | 041[TL1a]_H1_G44C_L1_A100C | | | |
| 53222 | IL23-A[IL23]_H1.169_L1.79_Fab- | 6.14E−11 | 6.39E+05 | 3.92E−05 |
| | 041[TL1a]_H1_G44C_L1_A100C | | | |
| 53223 | IL23-A[IL23]_H1.187_L1.69_Fab- | 2.84E−11 | 1.83E+06 | 5.18E−05 |
| | 041[TL1a]_H1_G44C_L1_A100C | | | |
| 53224 | IL23-A[IL23]_H1.187_L1.58_Fab- | 6.98E−11 | 1.06E+06 | 7.40E−05 |
| | 041[TL1a]_H1_G44C_L1_A100C | | | |
| 53225 | IL23-A[IL23]_H1.187_L1.79_Fab- | 6.57E−11 | 6.85E+05 | 4.50E−05 |
| | 041[TL1a]_H1_G44C_L1_A100C | | | |
| 53226 | IL23-A[IL23]_H1.195_L1.69_Fab- | 1.36E−09 | 1.59E+04 | 2.16E−05 |
| | 041[TL1a]_H1_G44C_L1_A100C | | | |
| 53227 | IL23-A[IL23]_H1.195_L1.58_Fab- | 1.49E−09 | 5.72E+04 | 8.52E−05 |
| | 041[TL1a]_H1_G44C_L1_A100C | | | |
| 53228 | IL23-A[IL23]_H1.195_L1.79_Fab- | 5.14E−10 | 1.58E+05 | 8.10E−05 |
| | 041[TL1a]_H1_G44C_L1_A100C | | | |
| 53282 | IL23-A[IL23]_H1_L1.117_Fab- | 1.46E−11 | 1.77E+06 | 2.58E−05 |
| | 041[TL1a]_H1_G44C_L1_A100C | | | |
| 53283 | IL23-A[IL23]_H1.41_L1.117_Fab- | 9.56E−12 | 1.75E+06 | 1.67E−05 |
| | 041[TL1a]_H1_G44C_L1_A100C | | | |
| 53284 | IL23-A[IL23]_H1.55_L1.117_Fab- | 1.33E−11 | 1.66E+06 | 2.21E−05 |
| | 041[TL1a]_H1_G44C_L1_A100C | | | |
| 53285 | IL23-A[IL23]_H1.94_L1.117_Fab- | 1.19E−11 | 1.81E+06 | 2.15E−05 |
| | 041[TL1a]_H1_G44C_L1_A100C | | | |
| 53286 | IL23-A[IL23]_H1.101_L1.117_Fab- | 1.38E−11 | 1.58E+06 | 2.18E−05 |
| | 041[TL1a]_H1_G44C_L1_A100C | | | |
| 53287 | IL23-A[IL23]_H1.113_L1.117_Fab- | 1.29E−11 | 1.76E+06 | 2.27E−05 |
| | 041[TL1a]_H1_G44C_L1_A100C | | | |
| 53288 | IL23-A[IL23]_H1.125_L1.117_Fab- | 1.37E−11 | 1.39E+06 | 1.90E−05 |
| | 041[TL1a]_H1_G44C_L1_A100C | | | |
| 53289 | IL23-A[IL23]_H1.140_L1.117_Fab- | 1.82E−11 | 1.19E+06 | 2.16E−05 |
| | 041[TL1a]_H1_G44C_L1_A100C | | | |
| 53290 | IL23-A[IL23]_H1.141_L1.117_Fab- | 1.14E−11 | 1.29E+06 | 1.47E−05 |
| | 041[TL1a]_H1_G44C_L1_A100C | | | |
| 53291 | IL23-A[IL23]_H1.142_L1.117_Fab- | 1.56E−11 | 1.30E+06 | 2.03E−05 |
| | 041[TL1a]_H1_G44C_L1_A100C | | | |
| 53292 | IL23-A[IL23]_H1.169_L1.117_Fab- | 1.58E−11 | 8.69E+05 | 1.37E−05 |
| | 041[TL1a]_H1_G44C_L1_A100C | | | |
| 53293 | IL23-A[IL23]_H1.187_L1.117_Fab- | 1.18E−11 | 1.57E+06 | 1.85E−05 |
| | 041[TL1a]_H1_G44C_L1_A100C | | | |
| 53294 | IL23-A[IL23]_H1.195_L1.117_Fab- | 1.61E−10 | 2.81E+05 | 4.51E−05 |
| | 041[TL1a]_H1_G44C_L1_A100C | | | |

The variants were first investigated in the 1+1 Fab-scFv-Fc format. Data depicting TL1A and IL23 inhibitory activities are depicted in Tables 41 and 42. Table 41 depicts IL23 inhibition IC₅₀ values for TL1A×IL-23 bispecific antibodies in the 1+1 Fab-scFv-Fc format, along with controls, incorporating affinity-engineered IL23-A variants and the parental 041. Table 42 depicts TL1A inhibition IC₅₀ values for TL1A×IL-23 bispecific antibodies in the 1+1 Fab-scFv-Fc format, along with controls, incorporating affinity-engineered 041 variants and the parental IL23-A. Notably, improved affinity did not always correspond to improved inhibitory activity, so this investigation was crucial for determining the top bispecific candidates.

TABLE 41

| | IC₅₀ (pM) |
|---|---|
| XENP53282 IL23 (IL23-A H1_L1.117) × | 12.65 |
| TL1a, 1 + 1 bottle-opener, FcKO Xtend | |
| XENP53288 IL23 (IL23-A H1.125_L1.117) × | 13.54 |
| TL1a, 1 + 1 bottle-opener, FcKO Xtend | |
| XENP53293 IL23 (IL23-A H1.187_L1.117) × | 13.78 |
| TL1a, 1 + 1 bottle-opener, FcKO Xtend | |
| XENP53291 IL23 (IL23-A H1.142_L1.117) × | 14.27 |
| TL1a, 1 + 1 bottle-opener, FcKO Xtend | |
| XENP53286 IL23 (IL23-A H1.101_L1.117) × | 16.42 |
| TL1a, 1 + 1 bottle-opener, FcKO Xtend | |
| XENP53292 IL23 (IL23-A H1.169_L1.117) × | 18.43 |
| TL1a, 1 + 1 bottle-opener, FcKO Xtend | |
| XENP53285 IL23 (IL23-A H1.94_L1.117) × | 18.59 |
| TL1a, 1 + 1 bottle-opener, FcKO Xtend | |
| XENP53214 IL23 (IL23-A H1.94_L1.69) × | 19.88 |
| TL1a, 1 + 1 bottle-opener, FcKO Xtend | |
| XENP53213 IL23 (IL23-A H1.101_L1.116) × | 21.18 |
| TL1a, 1 + 1 bottle-opener, FcKO Xtend | |
| XENP53211 IL23 (IL23-A H1.187_L1.116) × | 21.58 |
| TL1a, 1 + 1 bottle-opener, FcKO Xtend | |
| XENP53290 IL23 (IL23-A H1.141_L1.117) × | 21.95 |
| TL1a, 1 + 1 bottle-opener, FcKO Xtend | |
| XENP53287 IL23 (IL23-A H1.113_L1.117) × | 22.84 |
| TL1a, 1 + 1 bottle-opener, FcKO Xtend | |
| XENP53216 IL23 (IL23-A H1.94_L1.79) × | 24.62 |
| TL1a, 1 + 1 bottle-opener, FcKO Xtend | |
| XENP53217 IL23 (IL23-A H1.101_L1.69) × | 24.76 |
| TL1a, 1 + 1 bottle-opener, FcKO Xtend | |
| XENP53284 IL23 (IL23-A H1.55_L1.117) × | 26.41 |
| TL1a, 1 + 1 bottle-opener, FcKO Xtend | |
| XENP53289 IL23 (IL23-A H1.140_L1.117) × | 28.24 |
| TL1a, 1 + 1 bottle-opener, FcKO Xtend | |
| XENP53219 IL23 (IL23-A H1.101_L1.79) × | 28.7 |
| TL1a, 1 + 1 bottle-opener, FcKO Xtend | |
| XENP53223 IL23 (IL23-A H1.187_L1.69) × | 29.72 |
| TL1a, 1 + 1 bottle-opener, FcKO Xtend | |
| XENP53283 IL23 (IL23-A H1.41_L1.117) × | 30.66 |
| TL1a, 1 + 1 bottle-opener, FcKO Xtend | |
| XENP53220 IL23 (IL23-A H1.169_L1.69) × | 32.01 |
| TL1a, 1 + 1 bottle-opener, FcKO Xtend | |
| XENP53201 IL23 (IL23-A H1.113_L1) × | 32.13 |
| TL1a, 1 + 1 bottle-opener, FcKO Xtend | |
| XENP49698 IL23 (IL23-A H1L1), | 33.05 |
| bivalent, FcKO Xtend | |
| XENP53195 IL23 (IL23-A H1_L1.115) × | 33.23 |
| TL1a, 1 + 1 bottle-opener, FcKO Xtend | |
| XENP53225 IL23 (IL23-A H1.187_L1.79) × | 33.33 |
| TL1a, 1 + 1 bottle-opener, FcKO Xtend | |
| XENP53212 IL23 (IL23-A H1.169_L1.116) × | 35.16 |
| TL1a, 1 + 1 bottle-opener, FcKO Xtend | |
| XENP53196 IL23 (IL23-A H1_L1.116) × | 35.59 |
| TL1a, 1 + 1 bottle-opener, FcKO Xtend | |
| XENP53197 IL23 (IL23-A H1.41_L1) × | 36.11 |
| TL1a, 1 + 1 bottle-opener, FcKO Xtend | |
| XENP53207 IL23 (IL23-A H1.187_L1) × | 36.6 |
| TL1a, 1 + 1 bottle-opener, FcKO Xtend | |
| XENP53194 IL23 (IL23-A H1_L1.114) × | 36.99 |
| TL1a, 1 + 1 bottle-opener, FcKO Xtend | |
| XENP53210 IL23 (IL23-A H1.94_L1.116) × | 37.86 |
| TL1a, 1 + 1 bottle-opener, FcKO Xtend | |
| XENP53224 IL23 (IL23-A H1.187_L1.58) × | 40.73 |
| TL1a, 1 + 1 bottle-opener, FcKO Xtend | |
| XENP53200 IL23 (IL23-A H1.101_L1) × | 40.8 |
| TL1a, 1 + 1 bottle-opener, FcKO Xtend | |
| XENP53215 IL23 (IL23-A H1.94_L1.58) × | 42.6 |
| TL1a, 1 + 1 bottle-opener, FcKO Xtend | |
| XENP48697 IL23 (IL23-A H1L1 Fab) × | 42.73 |
| RSV, 1 + 1, Xtend FcKO | |
| XENP53222 IL23 (IL23-A H1.169_L1.79) × | 44.39 |
| TL1a, 1 + 1 bottle-opener, FcKO Xtend | |
| XENP53199 IL23 (IL23-A H1.94_L1) × | 48.64 |
| TL1a, 1 + 1 bottle-opener, FcKO Xtend | |
| XENP53198 IL23 (IL23-A H1.55_L1) × | 59.78 |
| TL1a, 1 + 1 bottle-opener, FcKO Xtend | |
| XENP53221 IL23 (IL23-A H1.169_L1.58) × | 104.1 |
| TL1a, 1 + 1 bottle-opener, FcKO Xtend | |
| XENP49699 IL23 (IL23-A H1L1) × | 109.9 |
| TL1a, 1 + 1 bottle-opener, FcKO Xtend | |

TABLE 42

| | IC₅₀ (nM) |
|---|---|
| XENP47942 041[TL1A]_H1L1 bivalent | 4.073 |
| FcKO Xtend | |
| XENP52385 041[TL1A]_H1.127_L1.27 × | 5.005 |
| IL23-A[IL23] 1 + 1 FcKO Xtend | |
| XENP52373 041[TL1A]_H1_L1.27 × | 5.048 |
| IL23-A[IL23] 1 + 1 FcKO Xtend | |
| XENP52386 041[TL1A]_H1.128_L1 × | 5.078 |
| IL23-A[IL23] 1 + 1 FcKO Xtend | |
| XENP52382 041[TL1A]_H1.127_L1 × | 5.284 |
| IL23-A[IL23] 1 + 1 FcKO Xtend | |
| XENP52387 041[TL1A]_H1.128_L1.25 × | 5.348 |
| IL23-A[IL23] 1 + 1 FcKO Xtend | |
| XENP52371 041[TL1A]_H1_L1.25 × | 5.375 |
| IL23-A[IL23] 1 + 1 FcKO Xtend | |
| XENP52372 041[TL1A]_H1_L1.26 × | 5.416 |
| IL23-A[IL23] 1 + 1 FcKO Xtend | |
| XENP52383 041[TL1A]_H1.127_L1.25 × | 5.759 |
| IL23-A[IL23] 1 + 1 FcKO Xtend | |
| XENP49320 041[TL1A]_H1L1 × | 5.764 |
| RSV1 + 1 FcKO Xtend | |
| XENP52389 041[TL1A]_H1.128_L1.27 × | 5.77 |
| IL23-A[IL23] 1 + 1 FcKO Xtend | |
| XENP52392 041[TL1A]_H1.132_L1.26 × | 5.797 |
| IL23-A[IL23] 1 + 1 FcKO Xtend | |
| XENP52393 041[TL1A]_H1.132_L1.27 × | 6.082 |
| IL23-A[IL23] 1 + 1 FcKO Xtend | |
| XENP52390 041[TL1A]_H1.132_L1 × | 6.252 |
| IL23-A[IL23] 1 + 1 FcKO Xtend | |
| XENP52396 041[TL1A]_H1_L1.76 × | 6.27 |
| IL23-A[IL23] 1 + 1 FcKO Xtend | |
| XENP52395 041[TL1A]_H1.115_L1.95 × | 6.343 |
| IL23-A[IL23] 1 + 1 FcKO Xtend | |
| XENP52388 041[TL1A]_H1.128_L1.26 × | 6.517 |
| IL23-A[IL23] 1 + 1 FcKO Xtend | |
| XENP49335 041[TL1A]_H1L1 × | 6.596 |
| IL23-A[IL23] 1 + 1 FcKO Xtend | |
| XENP52391 041[TL1A]_H1.132_L1.25 × | 7.004 |
| IL23-A[IL23] 1 + 1 FcKO Xtend | |
| XENP52374 041[TL1A]_H1.95_L1 × | 7.061 |
| IL23-A[IL23] 1 + 1 FcKO Xtend | |
| XENP52384 041[TL1A]_H1.127_L1.26 × | 7.63 |
| IL23-A[IL23] 1 + 1 FcKO Xtend | |
| XENP52394 041[TL1A]_H1.95_L1.76 × | 8.84 |
| IL23-A[IL23] 1 + 1 FcKO Xtend | |

### Example 6: Investigating Effect of Bispecific Formats

### 6A: TL1A and IL23 Inhibitory Activity

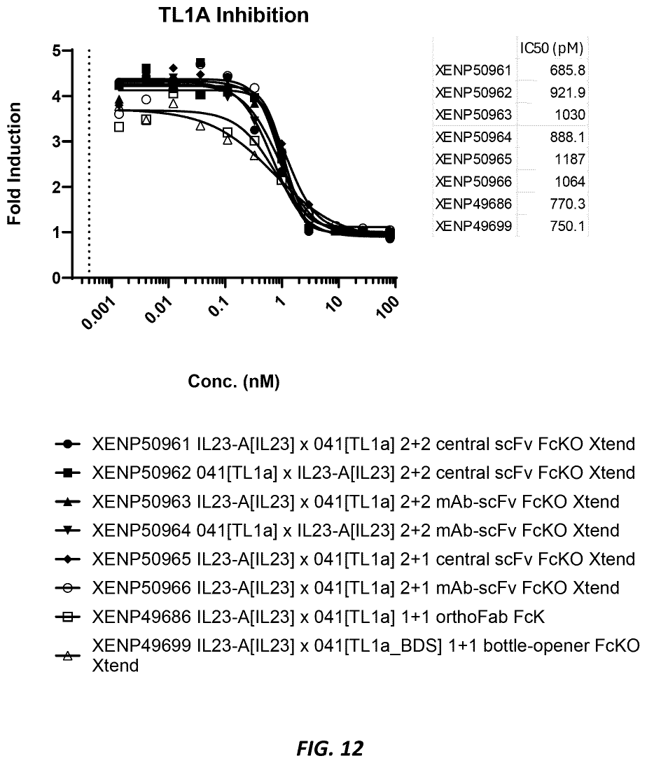

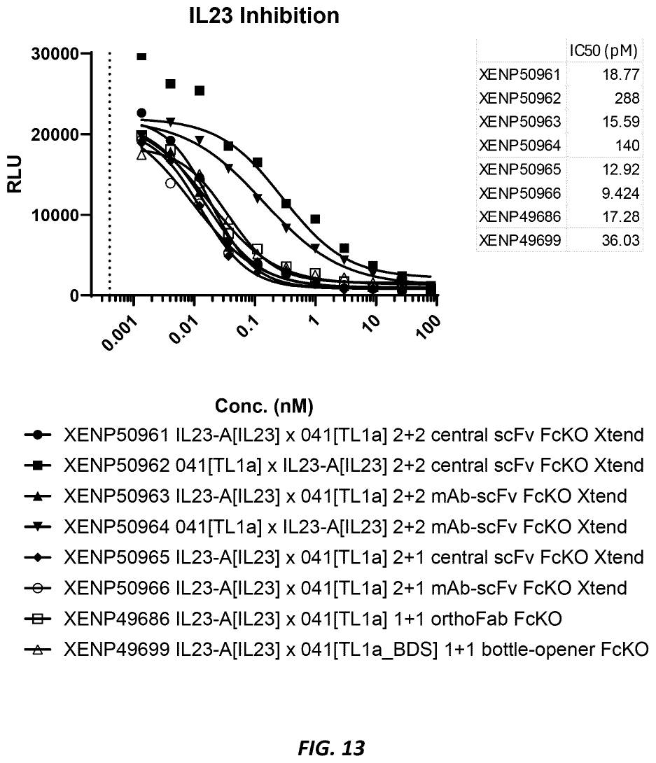

To determine whether different bispecific formats were preferable, additional formats described in Example 3 were investigated. Data depicting TL1A and IL23 inhibitory activity are depicted in FIGS. 12 and 13. Interestingly, the data shows that formats utilizing anti-IL23 as an scFv were less potent in inducing IL23 inhibition.

As the activities were otherwise comparable, formats such as the OrthoFab format and the CrossMab-based formats were preferred for their native IgG-like structures.

### 6B: Manufacturability

Surprisingly, between the OrthoFab and CrossMab, the OrthoFab was preferred, as at least in the context of the TL1A×IL23 bispecific antibodies, OrthoFab may be better when regarding manufacturability. As depicted in Table 43, transient expression (total protein produced) in CHO cells was superior for the exemplary bispecific antibodies in the OrthoFab format (XENP53369, XENP53375, XENP53412, and XENP53413) relative to exemplary bispecific antibodies in the CrossMab format (XENP53387 and XENP53415) shown in Table 44. The TL1A binding domains of XENP53412, XENP53415, and XENP53413 were derived from TL1A_041_H1.337_L1.27, the TL1A binding domains of XENP53387 and XENP53375 were derived from TL1A_041_H1.447_L1.27, and the TL1A binding domain of XENP53369 was derived from TL1A_041_H1.324_L1.27. The IL23 binding domains of XENP53412, XENP53415, XENP53387, XENP53375, and XENP53369 were derived from IL23-A_H1_L1.117, and the IL23 binding domain of XENP53413 was derived from IL23-A_H1.187_L1.116.

TABLE 43

| | Host Cell | Titer (mg/L) | Production Volume (L) |
|---|---|---|---|
| XENP53369 | CHO | 2855 | 10 |
| XENP53375 | CHO | 2871 | 10 |
| XENP53387 | CHO | 1986 | 10 |
| XENP53412 | CHO | 2576 | 10 |
| XENP53413 | CHO | 2721 | 10 |
| XENP53415 | CHO | 1577 | 10 |

TABLE 44

| | | SEQ |
|---|---|---|
| XENP53412 | sequence | ID NO: |
| Heavy Chain 1 | QVQLVQSGAEVKKPGASVKVSCKVSGFSVTSYNVHWVREAPGQGLEWMGV | 2240 |
| | MWYGGNTDYNSKFQGRVTMTRDTSTNTVYMELSSLRSEDTAVYYCARAGRY | |
| | YVDSNYYWDFPYWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQTYIC | |
| | NVNHKPSDTKVDEKVEPDSCDKTHTCPPCPAPPVAGPSVFLFPPKPKDTLMISR | |
| | TPEVTCVVVDVKHEDPEVKFNWYVDGVEVHNAKTKPREEEYNSTYRVVSVL | |
| | TVLHQDWLNGKEYKCKVSNKALPAPIEKTISKAKGQPREPQVYTLPPSREEMT | |
| | KNQVSLTCDVSGFYPSDIAVEWESDGQPENNYKTTPPVLDSDGSFFLYSKLTVD | |
| | KSRWEQGDVFSCSVLHEALHSHYTQKSLSLSPGK | |
| TL1A Variable | QVQLVQSGAEVKKPGASVKVSCKVSGFSVTSYNVHWVREAPGQGLEWMGV | 2241 |
| heavy (vh) domain | MWYGGNTDYNSKFQGRVTMTRDTSTNTVYMELSSLRSEDTAVYYCARAGRY | |
| | YVDSNYYWDFPYWGQGTLVTVSS | |
| vhCDR1 | SYNVH | 102 |
| vhCDR2 | VMWYGGNTDYNSKFQG | 1924 |
| vhCDR3 | AGRYYVDSNYYWDFPY | 1203 |
| Light Chain 1 | DIQMTQSPSSVSASVGDRVTITCRASEDIYSGLAWYQKKPGKSPKLLIYGASSL | 2242 |
| | QDGVPSRFSGSGSGTDYTLTISSLQPEDFATYFCQEGLKSPYTFGQGTKLEIKRT | |
| | VAAPSVFIFPPSKKQLKSGTASVVCLLNNFYPREAKVQWKVDNALQSGNSQES | |
| | VTEQDSKDSTYSLSSTLTLSKADYEKHKVYACEVTHQGLSSPVTKSFNRGEC | |
| TL1A Variable light | DIQMTQSPSSVSASVGDRVTITCRASEDIYSGLAWYQKKPGKSPKLLIYGASSL | 2243 |
| (vl) domain | QDGVPSRFSGSGSGTDYTLTISSLQPEDFATYFCQEGLKSPYTFGQGTKLEIK | |
| VlCDR1 | RASEDIYSGLA | 106 |
| VlCDR2 | GASSLQD | 107 |
| VlCDR3 | QEGLKSPYT | 108 |
| Heavy Chain 2 | QVQLVQSGAEVKKPGSSVKVSCKASGYTFTDQTIHWMRKAPGQGLEWIGYIY | 2244 |
| | PRDDSPKYNENFKGKVTITADKSTSTAYMELSSLRSEDTAVYYCAIPDRSGYAW | |
| | FIYWGQGTLVTVSSASTKGPSVFPLAPSSKSTSGGTAFLGCLVKDYFPEPVTVS | |
| | WNSGALTSGVHTFPAVLQSSGLYSLSSVVTVPSSSLGTQTYICNVNHKPSNTKV | |
| | DKKVEPKSCDKTHTCPPCPAPPVAGPSVFLFPPKPKDTLMISRTPEVTCVVVDV | |
| | KHEDPEVKFNWYVDGVEVHNAKTKPREEQYNSTYRVVSVLTVLHQDWLNG | |
| | KEYKCKVSNKALPAPIEKTISKAKGQPREPQVYTLPPSREQMTKNQVKLTCLV | |
| | KGFYPSDIAVEWESNGQPENNYKTTPPVLDSDGSFFLYSKLTVDKSRWQQGNV | |
| | FSCSVLHEALHSHYTQKSLSLSPGK | |
| IL23 Variable heavy | QVQLVQSGAEVKKPGSSVKVSCKASGYTFTDQTIHWMRKAPGQGLEWIGYIY | 2233 |
| (vh) domain | PRDDSPKYNENFKGKVTITADKSTSTAYMELSSLRSEDTAVYYCAIPDRSGYAW | |
| | FIYWGQGTLVTVSS | |
| vhCDR1 | DQTIH | 180 |
| vhCDR2 | YIYPRDDSPKYNENFKG | 181 |
| vhCDR3 | PDRSGYAWFIY | 182 |
| Light Chain 2 | DIQMTQSPSSLSASVGDRVTITCKASRDVAIAVAWYQEKPGKVPKLLIYWASVR | 2245 |
| | HTGVPSRFSGSGSRTDFTLTISSLQPEDVADYFCYQYSSYPFTFGSGTKLEIKRT | |
| | VAAPSVFIAPPSDEQLKSGTASVVCLLNNFYPREAKVQWKVDNALQSGNSQES | |
| | VTEQDSKDSTYSLSSTLTLSKADYEKHKVYACEVTHQGLSSPVTKSFNRGEC | |
| IL23 Variable light | DIQMTQSPSSLSASVGDRVTITCKASRDVAIAVAWYQEKPGKVPKLLIYWASVR | 2234 |
| (vl) domain | HTGVPSRFSGSGSRTDFTLTISSLQPEDVADYFCYQYSSYPFTFGSGTKLEIK | |
| VlCDR1 | KASRDVAIAVA | 184 |
| VlCDR2 | WASVRHT | 1249 |
| VlCDR3 | YQYSSYPFT | 1250 |
| Heavy Chain 1 | QVQLVQSGAEVKKPGASVKVSCKVSGFSVTSYNVHWVREAPGQGLEWMGV | 2240 |
| | MWYGGNTDYNSKFQGRVTMTRDTSTNTVYMELSSLRSEDTAVYYCARAGRY | |
| | YVDSNYYWDFPYWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQTYIC | |
| | NVNHKPSDTKVDEKVEPDSCDKTHTCPPCPAPPVAGPSVFLFPPKPKDTLMISR | |
| | TPEVTCVVVDVKHEDPEVKFNWYVDGVEVHNAKTKPREEEYNSTYRVVSVL | |
| | TVLHQDWLNGKEYKCKVSNKALPAPIEKTISKAKGQPREPQVYTLPPSREEMT | |
| | KNQVSLTCDVSGFYPSDIAVEWESDGQPENNYKTTPPVLDSDGSFFLYSKLTVD | |
| | KSRWEQGDVFSCSVLHEALHSHYTQKSLSLSPGK | |
| TL1A Variable | QVQLVQSGAEVKKPGASVKVSCKVSGFSVTSYNVHWVREAPGQGLEWMGV | 2241 |
| heavy (vh) domain | MWYGGNTDYNSKFQGRVTMTRDTSTNTVYMELSSLRSEDTAVYYCARAGRY | |
| | YVDSNYYWDFPYWGQGTLVTVSS | |
| vhCDR1 | SYNVH | 102 |
| vhCDR2 | VMWYGGNTDYNSKFQG | 1924 |
| vhCDR3 | AGRYYVDSNYYWDFPY | 1203 |
| Light Chain 1 | DIQMTQSPSSVSASVGDRVTITCRASEDIYSGLAWYQKKPGKSPKLLIYGASSL | 2242 |
| | QDGVPSRFSGSGSGTDYTLTISSLQPEDFATYFCQEGLKSPYTFGQGTKLEIKRT | |
| | VAAPSVFIFPPSKKQLKSGTASVVCLLNNFYPREAKVQWKVDNALQSGNSQES | |
| | VTEQDSKDSTYSLSSTLTLSKADYEKHKVYACEVTHQGLSSPVTKSFNRGEC | |
| TL1A Variable light | DIQMTQSPSSVSASVGDRVTITCRASEDIYSGLAWYQKKPGKSPKLLIYGASSL | 2243 |
| (vl) domain | QDGVPSRFSGSGSGTDYTLTISSLQPEDFATYFCQEGLKSPYTFGQGTKLEIK | |
| VlCDR1 | RASEDIYSGLA | 106 |
| VlCDR2 | GASSLQD | 107 |
| VlCDR3 | QEGLKSPYT | 108 |
| Heavy Chain 2 | QVQLVQSGAEVKKPGSSVKVSCKASGYTFTDQTIHWMRKAPGQGLEWIGYIY | 2250 |
| | PRDDSPKYNENFKGKVTITADKSTSTAYMELSSLRSEDTAVYYCAIPDRSGYAF | |
| | FIYWGQGTLVTVSSASTKGPSVFPLAPSSKSTSGGTAFLGCLVKDYFPEPVTVS | |
| | WNSGALTSGVHTFPAVLQSSGLYSLSSVVTVPSSSLGTQTYICNVNHKPSNTKV | |
| | DKKVEPKSCDKTHTCPPCPAPPVAGPSVFLFPPKPKDTLMISRTPEVTCVVVDV | |
| | KHEDPEVKFNWYVDGVEVHNAKTKPREEQYNSTYRVVSVLTVLHQDWLNG | |
| | KEYKCKVSNKALPAPIEKTISKAKGQPREPQVYTLPPSREQMTKNQVKLTCLV | |
| | KGFYPSDIAVEWESNGQPENNYKTTPPVLDSDGSFFLYSKLTVDKSRWQQGNV | |
| | FSCSVLHEALHSHYTQKSLSLSPGK | |
| IL23 Variable heavy | QVQLVQSGAEVKKPGSSVKVSCKASGYTFTDQTIHWMRKAPGQGLEWIGYIY | 2235 |
| (vh) domain | PRDDSPKYNENFKGKVTITADKSTSTAYMELSSLRSEDTAVYYCAIPDRSGYAF | |
| | FIYWGQGTLVTVSS | |
| vhCDR1 | DQTIH | 180 |
| vhCDR2 | YIYPRDDSPKYNENFKG | 181 |
| vhCDR3 | PDRSGYAFFIY | 2236 |
| Light Chain 2 | DIQMTQSPSSLSASVGDRVTITCKASRDVAIAVAWYQEKPGKVPKLLIYWASQR | 2251 |
| | HTGVPSRFSGSGSRTDFTLTISSLQPEDVADYFCYQYSSYPFTFGSGTKLEIKRT | |
| | VAAPSVFIAPPSDEQLKSGTASVVCLLNNFYPREAKVQWKVDNALQSGNSQES | |
| | VTEQDSKDSTYSLSSTLTLSKADYEKHKVYACEVTHQGLSSPVTKSFNRGEC | |
| IL23 Variable light | DIQMTQSPSSLSASVGDRVTITCKASRDVAIAVAWYQEKPGKVPKLLIYWASQR | 2237 |
| (vl) domain | HTGVPSRFSGSGSRTDFTLTISSLQPEDVADYFCYQYSSYPFTFGSGTKLEIK | |
| VlCDR1 | KASRDVAIAVA | 184 |
| VlCDR2 | WASQRHT | 2238 |
| VlCDR3 | YQYSSYPFT | 1250 |
| Heavy Chain 1 | QVQLVQSGAEVKKPGASVKVSCKVSGFSVTRYNVHWVREAPGQGLEWMGV | 2252 |
| | MWSGANTDYNSKFQGRVTMTRDTSTNTVYMELSSLRSEDTAVYYCARAGRY | |
| | YVDSNYYWDFPYWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQTYIC | |
| | NVNHKPSDTKVDEKVEPDSCDKTHTCPPCPAPPVAGPSVFLFPPKPKDTLMISR | |
| | TPEVTCVVVDVKHEDPEVKFNWYVDGVEVHNAKTKPREEEYNSTYRVVSVL | |
| | TVLHQDWLNGKEYKCKVSNKALPAPIEKTISKAKGQPREPQVYTLPPSREEMT | |
| | KNQVSLTCDVSGFYPSDIAVEWESDGQPENNYKTTPPVLDSDGSFFLYSKLTVD | |
| | KSRWEQGDVFSCSVLHEALHSHYTQKSLSLSPGK | |
| TL1A Variable | QVQLVQSGAEVKKPGASVKVSCKVSGFSVTRYNVHWVREAPGQGLEWMGV | 2253 |
| heavy (vh) domain | MWSGANTDYNSKFQGRVTMTRDTSTNTVYMELSSLRSEDTAVYYCARAGRY | |
| | YVDSNYYWDFPYWGQGTLVTVSS | |
| vhCDR1 | RYNVH | 1201 |
| vhCDR2 | VMWSGANTDYNSKFQG | 1202 |
| vhCDR3 | AGRYYVDSNYYWDFPY | 1203 |
| Light Chain 1 | DIQMTQSPSSVSASVGDRVTITCRASEDIYSGLAWYQKKPGKSPKLLIYGASSL | 2242 |
| | QDGVPSRFSGSGSGTDYTLTISSLQPEDFATYFCQEGLKSPYTFGQGTKLEIKRT | |
| | VAAPSVFIFPPSKKQLKSGTASVVCLLNNFYPREAKVQWKVDNALQSGNSQES | |
| | VTEQDSKDSTYSLSSTLTLSKADYEKHKVYACEVTHQGLSSPVTKSFNRGEC | |
| TL1A Variable light | DIQMTQSPSSVSASVGDRVTITCRASEDIYSGLAWYQKKPGKSPKLLIYGASSL | 2243 |
| (vl) domain | QDGVPSRFSGSGSGTDYTLTISSLQPEDFATYFCQEGLKSPYTFGQGTKLEIK | |
| VlCDR1 | RASEDIYSGLA | 106 |
| VlCDR2 | GASSLQD | 107 |
| VlCDR3 | QEGLKSPYT | 108 |
| Heavy Chain 2 | QVQLVQSGAEVKKPGSSVKVSCKASGYTFTDQTIHWMRKAPGQGLEWIGYIY | 2244 |
| | PRDDSPKYNENFKGKVTITADKSTSTAYMELSSLRSEDTAVYYCAIPDRSGYAW | |
| | FIYWGQGTLVTVSSASTKGPSVFPLAPSSKSTSGGTAFLGCLVKDYFPEPVTVS | |
| | WNSGALTSGVHTFPAVLQSSGLYSLSSVVTVPSSSLGTQTYICNVNHKPSNTKV | |
| | DKKVEPKSCDKTHTCPPCPAPPVAGPSVFLFPPKPKDTLMISRTPEVTCVVVDV | |
| | KHEDPEVKFNWYVDGVEVHNAKTKPREEQYNSTYRVVSVLTVLHQDWLNG | |
| | KEYKCKVSNKALPAPIEKTISKAKGQPREPQVYTLPPSREQMTKNQVKLTCLV | |
| | KGFYPSDIAVEWESNGQPENNYKTTPPVLDSDGSFFLYSKLTVDKSRWQQGNV | |
| | FSCSVLHEALHSHYTQKSLSLSPGK | |
| IL23 Variable heavy | QVQLVQSGAEVKKPGSSVKVSCKASGYTFTDQTIHWMRKAPGQGLEWIGYIY | 2233 |
| (vh) domain | PRDDSPKYNENFKGKVTITADKSTSTAYMELSSLRSEDTAVYYCAIPDRSGYAW | |
| | FIYWGQGTLVTVSS | |
| vhCDR1 | DQTIH | 180 |
| vhCDR2 | YIYPRDDSPKYNENFKG | 181 |
| vhCDR3 | PDRSGYAWFIY | 182 |
| Light Chain 2 | DIQMTQSPSSLSASVGDRVTITCKASRDVAIAVAWYQEKPGKVPKLLIYWASVR | 2245 |
| | HTGVPSRFSGSGSRTDFTLTISSLQPEDVADYFCYQYSSYPFTFGSGTKLEIKRT | |
| | VAAPSVFIAPPSDEQLKSGTASVVCLLNNFYPREAKVQWKVDNALQSGNSQES | |
| | VTEQDSKDSTYSLSSTLTLSKADYEKHKVYACEVTHQGLSSPVTKSFNRGEC | |
| IL23 Variable light | DIQMTQSPSSLSASVGDRVTITCKASRDVAIAVAWYQEKPGKVPKLLIYWASVR | 2234 |
| (vl) domain | HTGVPSRFSGSGSRTDFTLTISSLQPEDVADYFCYQYSSYPFTFGSGTKLEIK | |
| VlCDR1 | KASRDVAIAVA | 184 |
| VlCDR2 | WASVRHT | 1249 |
| VlCDR3 | YQYSSYPFT | 1250 |
| Heavy Chain 1 | QVQLVQSGAEVKKPGASVKVSCKVSGFPVTSYNVHWVREAPGQGLEWMGV | 2258 |
| | MWYGGNTDYNSKFQGRVTMTRDTSTNTVYMELSSLRSEDTAVYYCARAGRY | |
| | YHDSNYYWDFPYWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQTYIC | |
| | NVNHKPSDTKVDEKVEPDSCDKTHTCPPCPAPPVAGPSVFLFPPKPKDTLMISR | |
| | TPEVTCVVVDVKHEDPEVKFNWYVDGVEVHNAKTKPREEEYNSTYRVVSVL | |
| | TVLHQDWLNGKEYKCKVSNKALPAPIEKTISKAKGQPREPQVYTLPPSREEMT | |
| | KNQVSLTCDVSGFYPSDIAVEWESDGQPENNYKTTPPVLDSDGSFFLYSKLTVD | |
| | KSRWEQGDVFSCSVLHEALHSHYTQKSLSLSPGK | |
| TL1A Variable | QVQLVQSGAEVKKPGASVKVSCKVSGFPVTSYNVHWVREAPGQGLEWMGV | 2259 |
| heavy (vh) domain | MWYGGNTDYNSKFQGRVTMTRDTSTNTVYMELSSLRSEDTAVYYCARAGRY | |
| | YHDSNYYWDFPYWGQGTLVTVSS | |
| vhCDR1 | SYNVH | 102 |
| vhCDR2 | VMWYGGNTDYNSKFQG | 1924 |
| vhCDR3 | AGRYYHDSNYYWDFPY | 104 |
| Light Chain 1 | DIQMTQSPSSVSASVGDRVTITCRASEDIYSGLAWYQKKPGKSPKLLIYGASSL | 2242 |
| | QDGVPSRFSGSGSGTDYTLTISSLQPEDFATYFCQEGLKSPYTFGQGTKLEIKRT | |
| | VAAPSVFIFPPSKKQLKSGTASVVCLLNNFYPREAKVQWKVDNALQSGNSQES | |
| | VTEQDSKDSTYSLSSTLTLSKADYEKHKVYACEVTHQGLSSPVTKSFNRGEC | |
| TL1A Variable light | DIQMTQSPSSVSASVGDRVTITCRASEDIYSGLAWYQKKPGKSPKLLIYGASSL | 2243 |
| (vl) domain | QDGVPSRFSGSGSGTDYTLTISSLQPEDFATYFCQEGLKSPYTFGQGTKLEIK | |
| VlCDR1 | RASEDIYSGLA | 106 |
| VlCDR2 | GASSLQD | 107 |
| VlCDR3 | QEGLKSPYT | 108 |
| Heavy Chain 2 | QVQLVQSGAEVKKPGSSVKVSCKASGYTFTDQTIHWMRKAPGQGLEWIGYIY | 2244 |
| | PRDDSPKYNENFKGKVTITADKSTSTAYMELSSLRSEDTAVYYCAIPDRSGYAW | |
| | FIYWGQGTLVTVSSASTKGPSVFPLAPSSKSTSGGTAFLGCLVKDYFPEPVTVS | |
| | WNSGALTSGVHTFPAVLQSSGLYSLSSVVTVPSSSLGTQTYICNVNHKPSNTKV | |
| | DKKVEPKSCDKTHTCPPCPAPPVAGPSVFLFPPKPKDTLMISRTPEVTCVVVDV | |
| | KHEDPEVKFNWYVDGVEVHNAKTKPREEQYNSTYRVVSVLTVLHQDWLNG | |
| | KEYKCKVSNKALPAPIEKTISKAKGQPREPQVYTLPPSREQMTKNQVKLTCLV | |
| | KGFYPSDIAVEWESNGQPENNYKTTPPVLDSDGSFFLYSKLTVDKSRWQQGNV | |
| | FSCSVLHEALHSHYTQKSLSLSPGK | |
| IL23 Variable heavy | QVQLVQSGAEVKKPGSSVKVSCKASGYTFTDQTIHWMRKAPGQGLEWIGYIY | 2233 |
| (vh) domain | PRDDSPKYNENFKGKVTITADKSTSTAYMELSSLRSEDTAVYYCAIPDRSGYAW | |
| | FIYWGQGTLVTVSS | |
| vhCDR1 | DQTIH | 180 |
| vhCDR2 | YIYPRDDSPKYNENFKG | 181 |
| vhCDR3 | PDRSGYAWFIY | 182 |
| Light Chain 2 | DIQMTQSPSSLSASVGDRVTITCKASRDVAIAVAWYQEKPGKVPKLLIYWASVR | 2245 |
| | HTGVPSRFSGSGSRTDFTLTISSLQPEDVADYFCYQYSSYPFTFGSGTKLEIKRT | |
| | VAAPSVFIAPPSDEQLKSGTASVVCLLNNFYPREAKVQWKVDNALQSGNSQES | |
| | VTEQDSKDSTYSLSSTLTLSKADYEKHKVYACEVTHQGLSSPVTKSFNRGEC | |
| IL23 Variable light | DIQMTQSPSSLSASVGDRVTITCKASRDVAIAVAWYQEKPGKVPKLLIYWASVR | 2234 |
| (vl) domain | HTGVPSRFSGSGSRTDFTLTISSLQPEDVADYFCYQYSSYPFTFGSGTKLEIK | |
| VlCDR1 | KASRDVAIAVA | 184 |
| VlCDR2 | WASVRHT | 1249 |
| VlCDR3 | YQYSSYPFT | 1250 |
| Heavy Chain 1 | DIQMTQSPSSVSASVGDRVTITCRASEDIYSGLAWYQQKPGKSPKLLIYGASSL | 2264 |
| | QDGVPSRFSGSGSGTDYTLTISSLQPEDFATYFCQEGLKSPYTFGQGTKLEIKSS | |
| | ASTKGPSVFPLAPSSKSTSGGTAALGCLVKDYFPEPVTVSWNSGALTSGVHTFP | |
| | AVLQSSGLYSLSSVVTVPSSSLGTQTYICNVNHKPSDTKVDKKVEPKSCDKTH | |
| | TCPPCPAPPVAGPSVFLFPPKPKDTLMISRTPEVTCVVVDVKHEDPEVKFNWYV | |
| | DGVEVHNAKTKPREEEYNSTYRVVSVLTVLHQDWLNGKEYKCKVSNKALPA | |
| | PIEKTISKAKGQPREPQVYTLPPSREEMTKNQVSLTCDVSGFYPSDIAVEWESD | |
| | GQPENNYKTTPPVLDSDGSFFLYSKLTVDKSRWEQGDVFSCSVLHEALHSHYT | |
| | QKSLSLSPGK | |
| TL1A Variable light | DIQMTQSPSSVSASVGDRVTITCRASEDIYSGLAWYQQKPGKSPKLLIYGASSL | 1204 |
| (vl) domain | QDGVPSRFSGSGSGTDYTLTISSLQPEDFATYFCQEGLKSPYTFGQGTKLEIK | |
| vhCDR1 | RASEDIYSGLA | 106 |
| vhCDR2 | GASSLQD | 107 |
| vhCDR3 | QEGLKSPYT | 108 |
| Light Chain 1 | QVQLVQSGAEVKKPGASVKVSCKVSGFSVTSYNVHWVRQAPGQGLEWMGV | 2265 |
| | MWYGGNTDYNSKFQGRVTMTRDTSTNTVYMELSSLRSEDTAVYYCARAGRY | |
| | YVDSNYYWDFPYWGQGTLVTVSSASVAAPSVFIFPPSDEQLKSGTASVVCLLN | |
| | NFYPREAKVQWKVDNALQSGNSQESVTEQDSKDSTYSLSSTLTLSKADYEKH | |
| | KVYACEVTHQGLSSPVTKSFNRGEC | |
| TL1A Variable | QVQLVQSGAEVKKPGASVKVSCKVSGFSVTSYNVHWVRQAPGQGLEWMGV | 1674 |
| heavy (vh) domain | MWYGGNTDYNSKFQGRVTMTRDTSTNTVYMELSSLRSEDTAVYYCARAGRY | |
| | YVDSNYYWDFPYWGQGTLVTVSS | |
| VlCDR1 | SYNVH | 102 |
| VlCDR2 | VMWYGGNTDYNSKFQG | 1924 |
| VlCDR3 | AGRYYVDSNYYWDFPY | 1203 |
| Heavy Chain 2 | QVQLVQSGAEVKKPGSSVKVSCKASGYTFTDQTIHWMRQAPGQGLEWIGYIY | 2266 |
| | PRDDSPKYNENFKGKVTITADKSTSTAYMELSSLRSEDTAVYYCAIPDRSGYAW | |
| | FIYWGQGTLVTVSSASTKGPSVFPLAPSSKSTSGGTAALGCLVKDYFPEPVTVS | |
| | WNSGALTSGVHTFPAVLQSSGLYSLSSVVTVPSSSLGTQTYICNVNHKPSNTKV | |
| | DEKVEPDSCDKTHTCPPCPAPPVAGPSVFLFPPKPKDTLMISRTPEVTCVVVDV | |
| | KHEDPEVKFNWYVDGVEVHNAKTKPREEQYNSTYRVVSVLTVLHQDWLNG | |
| | KEYKCKVSNKALPAPIEKTISKAKGQPREPQVYTLPPSREQMTKNQVKLTCLV | |
| | KGFYPSDIAVEWESNGQPENNYKTTPPVLDSDGSFFLYSKLTVDKSRWQQGNV | |
| | FSCSVLHEALHSHYTQKSLSLSPGK | |
| IL23 Variable heavy | QVQLVQSGAEVKKPGSSVKVSCKASGYTFTDQTIHWMRQAPGQGLEWIGYIY | 179 |
| (vh) domain | PRDDSPKYNENFKGKVTITADKSTSTAYMELSSLRSEDTAVYYCAIPDRSGYAW | |
| | FIYWGQGTLVTVSS | |
| vhCDR1 | DQTIH | 180 |
| vhCDR2 | YIYPRDDSPKYNENFKG | 181 |
| vhCDR3 | PDRSGYAWFIY | 182 |
| Light Chain 2 | DIQMTQSPSSLSASVGDRVTITCKASRDVAIAVAWYQQKPGKVPKLLIYWASVR | 2267 |
| | HTGVPSRFSGSGSRTDFTLTISSLQPEDVADYFCYQYSSYPFTFGSGTKLEIKRT | |
| | VAAPSVFIFPPSKKQLKSGTASVVCLLNNFYPREAKVQWKVDNALQSGNSQES | |
| | VTEQDSKDSTYSLSSTLTLSKADYEKHKVYACEVTHQGLSSPVTKSFNRGEC | |
| IL23 Variable light | DIQMTQSPSSLSASVGDRVTITCKASRDVAIAVAWYQQKPGKVPKLLIYWASVR | 1248 |
| (vl) domain | HTGVPSRFSGSGSRTDFTLTISSLQPEDVADYFCYQYSSYPFTFGSGTKLEIK | |
| VlCDR1 | KASRDVAIAVA | 184 |
| VlCDR2 | WASVRHT | 1249 |
| VlCDR3 | YQYSSYPFT | 1250 |
| Heavy Chain 1 | DIQMTQSPSSVSASVGDRVTITCRASEDIYSGLAWYQQKPGKSPKLLIYGASSL | 2264 |
| | QDGVPSRFSGSGSGTDYTLTISSLQPEDFATYFCQEGLKSPYTFGQGTKLEIKSS | |
| | ASTKGPSVFPLAPSSKSTSGGTAALGCLVKDYFPEPVTVSWNSGALTSGVHTFP | |
| | AVLQSSGLYSLSSVVTVPSSSLGTQTYICNVNHKPSDTKVDKKVEPKSCDKTH | |
| | TCPPCPAPPVAGPSVFLFPPKPKDTLMISRTPEVTCVVVDVKHEDPEVKFNWYV | |
| | DGVEVHNAKTKPREEEYNSTYRVVSVLTVLHQDWLNGKEYKCKVSNKALPA | |
| | PIEKTISKAKGQPREPQVYTLPPSREEMTKNQVSLTCDVSGFYPSDIAVEWESD | |
| | GQPENNYKTTPPVLDSDGSFFLYSKLTVDKSRWEQGDVFSCSVLHEALHSHYT | |
| | QKSLSLSPGK | |
| TL1A Variable light | DIQMTQSPSSVSASVGDRVTITCRASEDIYSGLAWYQQKPGKSPKLLIYGASSL | 1204 |
| (vl) domain | QDGVPSRFSGSGSGTDYTLTISSLQPEDFATYFCQEGLKSPYTFGQGTKLEIK | |
| vhCDR1 | RASEDIYSGLA | 106 |
| vhCDR2 | GASSLQD | 107 |
| vhCDR3 | QEGLKSPYT | 108 |
| Light Chain 1 | QVQLVQSGAEVKKPGASVKVSCKVSGFSVTRYNVHWVRQAPGQGLEWMGV | 2269 |
| | MWSGANTDYNSKFQGRVTMTRDTSTNTVYMELSSLRSEDTAVYYCARAGRY | |
| | YVDSNYYWDFPYWGQGTLVTVSSASVAAPSVFIFPPSDEQLKSGTASVVCLLN | |
| | NFYPREAKVQWKVDNALQSGNSQESVTEQDSKDSTYSLSSTLTLSKADYEKH | |
| | KVYACEVTHQGLSSPVTKSFNRGEC | |
| TL1A Variable | QVQLVQSGAEVKKPGASVKVSCKVSGFSVTRYNVHWVRQAPGQGLEWMGV | 1200 |
| heavy (vh) domain | MWSGANTDYNSKFQGRVTMTRDTSTNTVYMELSSLRSEDTAVYYCARAGRY | |
| | YVDSNYYWDFPYWGQGTLVTVSS | |
| VlCDR1 | RYNVH | 1201 |
| VlCDR2 | VMWSGANTDYNSKFQG | 1202 |
| VlCDR3 | AGRYYVDSNYYWDFPY | 1203 |
| Heavy Chain 2 | QVQLVQSGAEVKKPGSSVKVSCKASGYTFTDQTIHWMRQAPGQGLEWIGYIY | 2266 |
| | PRDDSPKYNENFKGKVTITADKSTSTAYMELSSLRSEDTAVYYCAIPDRSGYAW | |
| | FIYWGQGTLVTVSSASTKGPSVFPLAPSSKSTSGGTAALGCLVKDYFPEPVTVS | |
| | WNSGALTSGVHTFPAVLQSSGLYSLSSVVTVPSSSLGTQTYICNVNHKPSNTKV | |
| | DEKVEPDSCDKTHTCPPCPAPPVAGPSVFLFPPKPKDTLMISRTPEVTCVVVDV | |
| | KHEDPEVKFNWYVDGVEVHNAKTKPREEQYNSTYRVVSVLTVLHQDWLNG | |
| | KEYKCKVSNKALPAPIEKTISKAKGQPREPQVYTLPPSREQMTKNQVKLTCLV | |
| | KGFYPSDIAVEWESNGQPENNYKTTPPVLDSDGSFFLYSKLTVDKSRWQQGNV | |
| | FSCSVLHEALHSHYTQKSLSLSPGK | |
| IL23 Variable heavy | QVQLVQSGAEVKKPGSSVKVSCKASGYTFTDQTIHWMRQAPGQGLEWIGYIY | 179 |
| (vh) domain | PRDDSPKYNENFKGKVTITADKSTSTAYMELSSLRSEDTAVYYCAIPDRSGYAW | |
| | FIYWGQGTLVTVSS | |
| vhCDR1 | DQTIH | 180 |
| vhCDR2 | YIYPRDDSPKYNENFKG | 181 |
| vhCDR3 | PDRSGYAWFIY | 182 |
| Light Chain 2 | DIQMTQSPSSLSASVGDRVTITCKASRDVAIAVAWYQQKPGKVPKLLIYWASVR | 2267 |
| | HTGVPSRFSGSGSRTDFTLTISSLQPEDVADYFCYQYSSYPFTFGSGTKLEIKRT | |
| | VAAPSVFIFPPSKKQLKSGTASVVCLLNNFYPREAKVQWKVDNALQSGNSQES | |
| | VTEQDSKDSTYSLSSTLTLSKADYEKHKVYACEVTHQGLSSPVTKSFNRGEC | |
| IL23 Variable light | DIQMTQSPSSLSASVGDRVTITCKASRDVAIAVAWYQQKPGKVPKLLIYWASVR | 1248 |
| (vl) domain | HTGVPSRFSGSGSRTDFTLTISSLQPEDVADYFCYQYSSYPFTFGSGTKLEIK | |
| VlCDR1 | KASRDVAIAVA | 184 |
| VlCDR2 | WASVRHT | 1249 |
| VlCDR3 | YQYSSYPFT | 1250 |

Further, in initial production of XENP49749 (OrthoFab) and XENP51595 (CrossMab) with the same DNA ratio (HC1:HC2:LC1:LC2 1:1:1:2), it was found that XENP51595 resulted in ~45% contaminating products (e.g., correct heavy chain pairing, incorrect light chain pairing) in comparison to XENP49749 which resulted in ~21% contaminating products.

### Example 7: Further Characterization of Exemplary TL1A×IL23 Bispecific Antibodies

The affinity-enhanced binding domains engineered as described in Example 5 were incorporated into TL1A×IL23 bispecific antibodies and further characterized as follows.

### 7A: TL1A and IL23 Binding Affinities

TL1A and IL23 binding affinities were generated under improved assay conditions to provide more accurate and reliable determinations than earlier preliminary data generated for early screening purposes. Data are depicted in Tables 45 and 46. Table 45 depicts binding kinetics as measured by Biacore at 37° C. for exemplary TL1A×IL23 bispecific antibodies with respect to TL1A, generated under improved assay conditions to provide more accurate and reliable determinations than earlier preliminary data. Table 46 depicts binding kinetics as measured by Biacore at 37° C. for exemplary TL1A×IL23 bispecific antibodies with respect to IL23, generated under improved assay conditions to provide more accurate and reliable determinations than earlier preliminary data. In the end, 041 variant H1.337_L1.27 and IL23-A variant H1_L1.117 were selected as favorites by virtue of having the tightest binding to their respective antigens. The TL1A binding domains of XENP53412, XENP53415, and XENP53413 were derived from 041 variant H1.337_L1.27, and the IL23 binding domains of XENP53412, XENP53415, XENP53387, XENP53375, and XENP53369 were derived from IL23-A variant H1_L1.117.

TABLE 45

| | | TL1A | | |
|---|---|---|---|---|
| | | average | | |
| | | kinetics | | |
| | | N = 3 | | |
| XENP | Description | K_{D} (M) | kₐ (1/Ms) | k_{d} (1/s) |
| XENP53369 | TL1a (041 H1.324_L1.27 orthoFab) × IL23 (IL23-A | 4.27E−11 | 6.09E+05 | 2.60E−05 |
| | H1_L1.117 orthoFab), 1 + 1 orthoFab, Xtend FcKO | | | |
| XENP53375 | TL1a (041 H1.447_L1.27 orthoFab) × IL23 (IL23-A | 6.22E−11 | 9.92E+05 | 6.17E−05 |
| | H1_L1.117 orthoFab), 1 + 1 orthoFab, Xtend FcKO | | | |
| XENP53412 | TL1a (041 H1.337_L1.27 orthoFab) × IL23 (IL23-A | 2.61E−11 | 5.86E+05 | 1.53E−05 |
| | H1_L1.117 orthoFab), 1 + 1 orthoFab, Xtend FcKO | | | |
| XENP53413 | TL1a (041 H1.337_L1.27 orthoFab) × IL23 (IL23-A | 2.56E−11 | 5.75E+05 | 1.47E−05 |
| | H1.187_L1.116 orthoFab), 1 + 1 orthoFab, Xtend | | | |
| | FcKO | | | |
| XENP53387 | TL1a (041 L1.27-Fc H1.447-Ck) × IL23 (IL23-A | 6.22E−11 | 9.63E+05 | 5.98E−05 |
| | H1_K213E/K218D_L1.117_D122K/E123K_Fab), | | | |
| | 1 + 1 CrossMab-VH-VL, FcKO Xtend | | | |
| XENP53415 | TL1a (041 L1.27-Fc H1.337-Ck) × IL23 (IL23-A | 2.55E−11 | 6.13E+05 | 1.56E−05 |
| | H1_K213E/K218D_L1.117_D122K/E123K_Fab), | | | |
| | 1 + 1 CrossMab-VH-VL, FcKO Xtend | | | |
| XENP49335 | TL1a (041 H1L1 Fab) × IL23 (IL23-A H1L1 scFv), | 2.05E−10 | 1.09E+06 | 2.23E−04 |
| | 1 + 1 Fab-scFv-Fc, Xtend FcKO | | | |

TABLE 46

| | | IL23 average | | |
|---|---|---|---|---|
| | | kinetics | | |
| | | N = 3 | | |
| XENP | Description | K_{D} (M) | kₐ (1/Ms) | k_{d} (1/s) |
| XENP53369 | TL1a (041 H1.324_L1.27 orthoFab) × IL23 (IL23-A | 5.47E−11 | 4.58E+05 | 2.44E−05 |
| | H1_L1.117 orthoFab), 1 + 1 orthoFab, Xtend FcKO | | | |
| XENP53375 | TL1a (041 H1.447_L1.27 orthoFab) × IL23 (IL23-A | 2.68E−11 | 8.35E+05 | 2.19E−05 |
| | H1_L1.117 orthoFab), 1 + 1 orthoFab, Xtend FcKO | | | |
| XENP53412 | TL1a (041 H1.337_L1.27 orthoFab) × IL23 (IL23-A | 5.67E−11 | 4.33E+05 | 2.40E−05 |
| | H1_L1.117 orthoFab), 1 + 1 orthoFab, Xtend FcKO | | | |
| XENP53413 | TL1a (041 H1.337_L1.27 orthoFab) × IL23 (IL23-A | 1.02E−10 | 4.00E+05 | 3.99E−05 |
| | H1.187_L1.116 orthoFab), 1 + 1 orthoFab, Xtend | | | |
| | FcKO | | | |
| XENP53387 | TL1a (041 L1.27-Fc H1.447-Ck) × IL23 (IL23-A | 2.47E−11 | 8.20E+05 | 1.99E−05 |
| | H1_K213E/K218D_L1.117_D122K/E123K_Fab), | | | |
| | 1 + 1 CrossMab-VH-VL, FcKO Xtend | | | |
| XENP53415 | TL1a (041 L1.27-Fc H1.337-Ck) × IL23 (IL23-A | 5.42E−11 | 4.43E+05 | 2.32E−05 |
| | H1_K213E/K218D_L1.117_D122K/E123K_Fab), | | | |
| | 1 + 1 CrossMab-VH-VL, FcKO Xtend | | | |
| XENP49699 | IL23 (IL23-A H1L1) × TL1a (041 | 7.45E−11 | 9.25E+05 | 6.77E−05 |
| | H1_G44C_L1_A100C_scFv(GKPGS)4), 1 + 1 Fab- | | | |
| | scFv-Fc, FcKO Xtend | | | |

### 7B: TL1A and IL23 Inhibitory Activity

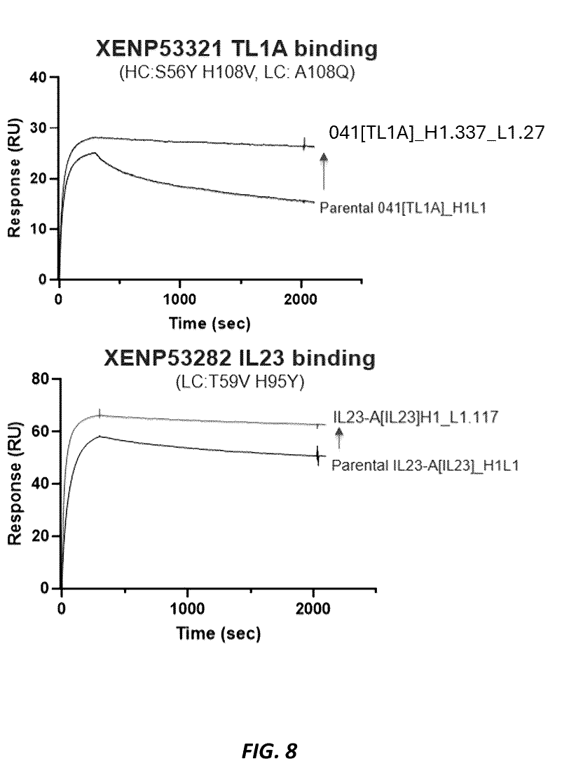

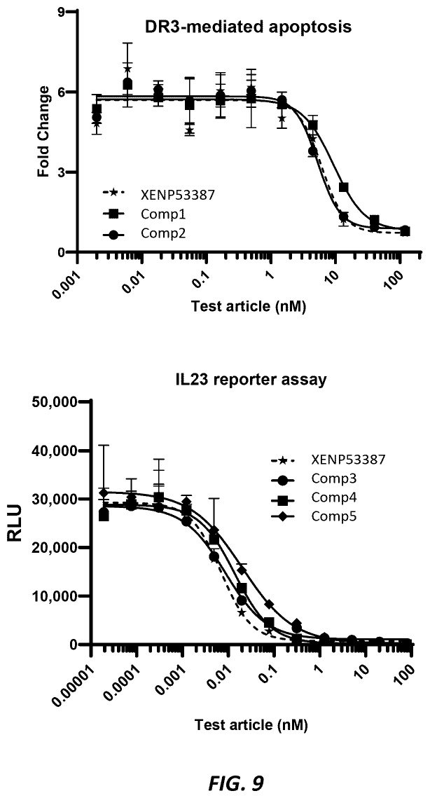

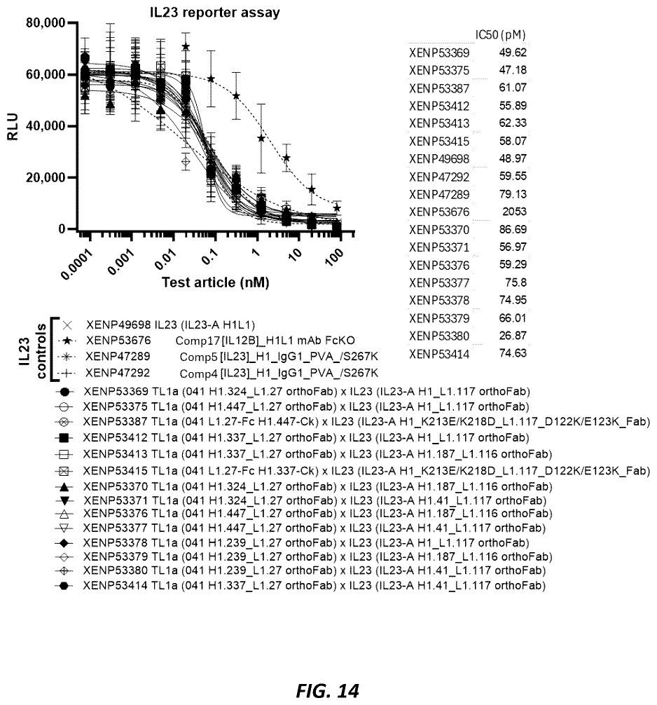

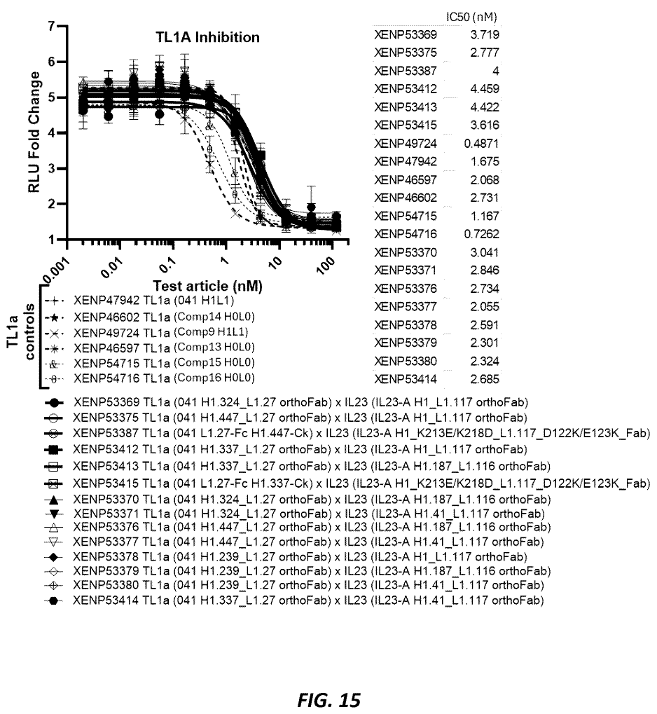

TL1A and TL23 inhibitory activity were investigated as described above, data for which are depicted in FIGS. 14 and 15. Inhibitory activity of an individual Fab binding domain within a bispecific modality would generally be expected to be ~50% less than a bivalent antibody variant in standard IgG format since binding reflects monovalent versus bivalent neutralization ability. However, as further shown in FIG. 9, the TL1A×IL23 bsAb XENP53387 antibody showed activity comparable to various bivalent TL1A and IL23 control comparator mAbs using an IL23 luciferase reporter assay as an indicator of IL23 activity and using a caspase 3/7 luminescence assay as an indicator of TL1A-induced apoptosis and TL1A activity (Tables 47-50). As shown in FIGS. 14 and 15, XENP53369, XENP53375, XENP53412, XENP53413, and XENP53415 had comparable activity to XENP53387. Table 47 depicts bispecific sequences utilizing TL1A and RSV or IL23 and RSV. Table 48 depicts sequences for bivalent TL1A mAbs utilized as controls. Table 49 depicts sequences for bivalent IL23 mAbs utilized as controls. Table 50 depicts sequences for bivalent IL12 mAbs utilized as controls.

TABLE 47

| | SEQ ID NO \| Antigen | | | | | |
|---|---|---|---|---|---|---|
| Xencor ID | Chain 1 | | Chain 2 | | Chain 3 | |
| XENP48697 | 1399 | IL23 | 1400 | RSV | 1401 | IL23 |
| XENP49320 | 1402 | TL1A | 1403 | RSV | 1404 | TL1A |

TABLE 48

SEQ ID NO | Antigen

| Xencor ID | Heavy Chain | | Light Chain | |
|---|---|---|---|---|
| XENP46597 | 1405 | TL1A | 1406 | TL1A |
| XENP46602 | 1407 | TL1A | 1408 | TL1A |
| XENP49724 | 1409 | TL1A | 1410 | TL1A |
| XENP54715 | 1411 | TL1A | 1412 | TL1A |
| XENP54716 | 1413 | TL1A | 1414 | TL1A |
| XENP47772 | 1415 | TL1A | 1416 | TL1A |
| XENP47824 | 1417 | TL1A | 1418 | TL1A |
| XENP47942 | 1419 | TL1A | 1420 | TL1A |

TABLE 49

SEQ ID NO | Antigen

| | | |
|---|---|---|
| Xencor ID | Heavy Chain | Light Chain |

| | | | | |
|---|---|---|---|---|
| XENP47289 | 1421 | IL23 | 1422 | IL23 |
| XENP47292 | 1423 | IL23 | 1424 | IL23 |

TABLE 50

SEQ ID NO | Antigen

| Xencor ID | Heavy Chain | | Light Chain | |
|---|---|---|---|---|
| XENP53676 | 1425 | IL12B | 1426 | IL 12B |

### 7C: In Vivo Pharmacokinetics

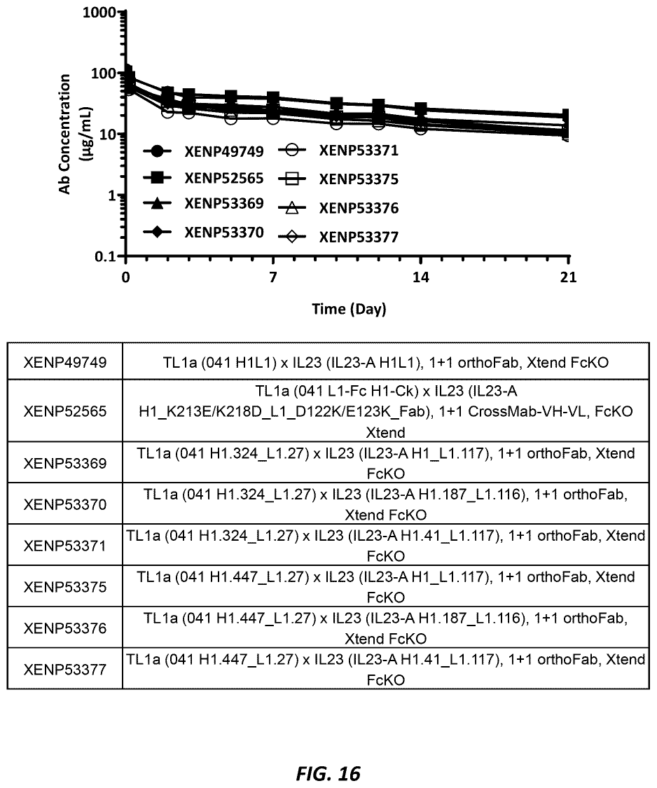

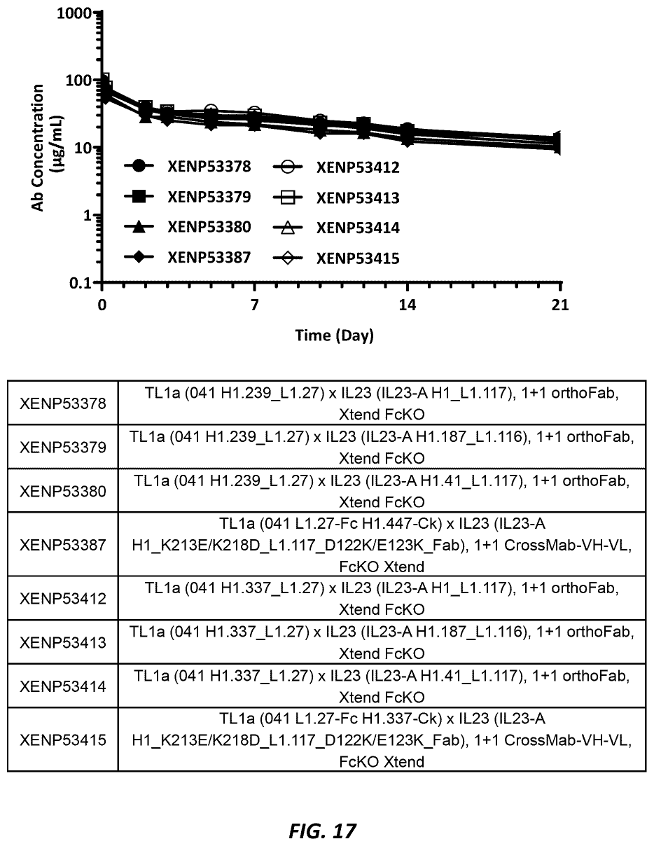

The pharmacokinetic profile of the TL1A×IL23 bispecific antibodies engineered with Fc variants for extended serum half-life were investigated in huFcRn mice. Mice were injected intravenously with 5 mg/kg TL1A×IL23 bispecific antibodies, and serum concentration was assessed over time, data for which are depicted in FIGS. 16 and 17. The data show good half-life for all bispecific antibodies.

### Example 8: Developability of Exemplary TL1A×IL23 Bispecific Antibodies

As the TL1A×IL23 bispecific antibodies described herein are intended, in part, for treatment of chronic diseases, it is desirable that the antibodies be deliverable by subcutaneous administration and exhibit extended serum half-life.

### 8A: Stability of TL1A×IL23 Bispecific Antibodies

To ensure suitability for subcutaneous administration, the bispecific antibodies must be stable (high melting temperature) and maintain low viscosity at high concentrations.

Stability of the bispecific antibodies were assessed using Differential Scanning Calorimetry (DSC), data for which are depicted in Table 51. All of the bispecific antibodies show a similar Tonset/Tm1 (CH2 domain) with all Tm2 being ≥75° C. and in a good thermostable range.

TABLE 51

| XENP | Tonset | Tm1 | Tm2 | Tm3 |
|---|---|---|---|---|
| 53369 | 57.19 | 65.68 | 79.61 | 83.79 |
| 53375 | 57.08 | 65.65 | 79.66 | 83.79 |
| 53387 | 57.88 | 65.87 | 77.62 | 80.66 |
| 53412 | 57.26 | 65.71 | 79.5 | 83.78 |
| 53413 | 58.05 | 65.78 | 79.36 | 82.59 |
| 53415 | 58.15 | 65.87 | 77.81 | 80.86 |
| 49698 | 58.44 | 65.64 | 77.95 | 83.32 |
| 47942 | 58.26 | 65.63 | 75.91 | 84.37 |

Next, the viscosity of the bispecific antibodies at high concentration (~150 mg/ml) at 20° C. was assessed, data for which are depicted in Table 52. Typically, viscosity over 20 mPa-s is considered high risk. Most of the bispecific antibodies show viscosity below 20 mPa-s.

TABLE 52

| XENP | mg/mL | mPa-s |
|---|---|---|
| 49749 | 156 | 5 |
| 52515 | 168 | 7.8 |
| 52516 | 152 | 8.9 |
| 52517 | 165 | 7.9 |
| 52565 | 176 | 15.4 |
| 52566 | 164 | 14.3 |
| 52877 | 184 | 31.1 |

### 8B: ADA Potential of TL1A×IL23 Bispecific Antibodies

While the bispecific antibodies were engineered with Fc variants to extend serum half-life and demonstrated favorable pharmacokinetics (PK) in huFcRn mice (see Example 6B), it is critical that they do not elicit anti-drug antibodies (ADA), as ADA responses could neutralize the therapeutic antibody or accelerate its clearance, thereby reducing half-life.

Pre-existing antibodies are naturally occurring immunoglobulins present prior to administration of a therapeutic drug. To minimize ADA concerns, candidate antibodies may be selected for minimal pre-existing reactivity. The pre-existing reactivity approach described by Bivi et al., 2019 (MAbs 11(5):861-869, doi:10.1080/19420862.2019.1612699) was applied to assess the TL1A×IL23 bispecific antibodies. As depicted in Table 53, each of the TL1A×IL23 bispecific antibodies tested have p90 values ranging from 12 to 21 which is on par with approved products such as pembrolizumab (p90=22) having low clinical ADA prevalence (0.7-2.5%).

TABLE 53

| XENP | p90 |
|---|---|
| 53369 | 20 |
| 53375 | 12 |
| 53387 | 12 |
| 53412 | 17 |
| 53413 | 19 |
| 53415 | 21 |

### Example 9: Evaluation of TL1A×IL23 Bispecific Antibodies in Colitis Model

The in vivo efficacy of TL1A×IL23 bispecific antibodies is investigated using a TNBS-induced acute colitis model in hTL1A/hIL-23A/hIL12B knock-in mice. At Day 0, colitis is induced in mice by intracolonic administration of 2% TNBS solution (0.1 mL), while sham animals receive 50% ethanol. Test articles (TL1A mAb, IL23 mAb, TL1A mAb+IL23 mAb, or TL1A×IL23 bispecific mAb) or vehicle are administered, and animals are monitored daily for clinical signs, including body weight, stool consistency, and fecal blood. The Disease Activity Index (DAI), comprising weight loss, stool score, and bleeding subscores, is recorded under blinded assessment. It is anticipated that administration of the test articles will result in reduced DAI scores compared to vehicle-treated controls, indicating therapeutic benefit.

At the study endpoint (Day 5), serum samples are collected for pharmacokinetic assessment, and colons are harvested for gross evaluation, including colon weight, length, and photographic documentation. It is expected that mice receiving the test articles will exhibit reduced colon weight-to-length ratios and attenuated macroscopic colonic damage relative to controls, supporting the conclusion that the test articles have anti-inflammatory activity and therapeutic potential for the treatment of inflammatory bowel disease.

### Example 10: Bivalent TL1A and IL23 mAbs

Although the foregoing examples describe TL1A×IL23 bispecific antibodies, any of the TL1A binding domains described in Example 1 and IL23 binding domains described in Example 2 may be formatted as bivalent monospecific antibodies and used alone or in combination.

### Means Plus Function Embodiments

### Group 1 “Means” Clauses: Means for Binding TL1A and Means for Binding IL23 Together

### Exemplary Clauses:

1. A bispecific antibody comprising: (a) a means for binding TL1A; and (b) a means for binding IL-23.
2. A pharmaceutical composition comprising: (a) the bispecific antibody of clause 1; and (b) a pharmaceutically acceptable carrier.
3. A pharmaceutical composition comprising: (a) a first composition comprising a means for binding TL1A; and (b) a second composition comprising a means for binding IL-23.
4. A pharmaceutical composition comprising: (a) a means for binding TL1A; (b) a means for binding IL23; and (c) a pharmaceutically acceptable carrier.

TABLE 54
below links the above listed Group 1 “means” clauses to the relevant portions of the specification.

| Recited | Exemplified structures and/or materials and/or acts disclosed in the Specification of the |
|---|---|
| function | application filed herewith |
| Means | Table 22: anti-TL1A antibody with HCDRs/LCDRs or VH/VL sequences of |
| for | “041_H1.337_L1.27,” “041_H1.447_L1.27,” “041_H1.324_L1.27,” or “041_H1.239_L1.27.” |
| binding | Table 44: anti-TL1A antibody with HCDRs/LCDRs, VH/VL, or heavy chain/light chain |
| TL1A | sequences of “XENP53412,” “XENP53413,” “XENP53375,” “XENP53369,” “XENP53415,” or |
| | “XENP53387.” |
| | Table 7: anti-TL1A antibody with HCDRs/LCDRs or VH/VL sequences of “041[TL1a]_H1L1.” |
| Means | Table 24: anti-IL23 antibody with HCDRs/LCDRs or VH/VL sequences of “IL23- |
| for | A_H1_L1.117,” “IL23-A_H1.187_L1.116,” or “IL23-A_H1.41_L1.117.” |
| binding | Table 44: anti-IL23 antibody with HCDRs/LCDRs, VH/VL, or heavy chain/light chain |
| IL23 | sequences of “XENP53412,” “XENP53413,” “XENP53375,” “XENP53369,” “XENP53415,” or |
| | “XENP53387.” |

### Group 2 “Means” Clauses: Means for Binding IL23, but TL1A Binding Domain Structurally Defined

### Exemplary Clauses:

1. An antibody comprising a TL1A antigen-binding domain comprising a variable heavy domain comprising a vhCDR1, a vhCDR2, and a vhCDR3 contained within a variable heavy domain comprising the amino acid sequence of SEQ ID NO: __, and a variable light domain comprising a vlCDR1, a vlCDR2, and a vlCDR3 contained within a variable light domain comprising the amino acid sequence of SEQ ID NO: __.
2. An antibody comprising a TL1A antigen-binding domain comprising a variable heavy domain comprising a vhCDR1 comprising the amino acid sequence of SEQ ID NO: __, a vhCDR2 comprising the amino acid sequence of SEQ ID NO: __, and a vhCDR3 comprising the amino acid sequence of SEQ ID NO: __, and a variable light domain comprising a vlCDR1 comprising the amino acid sequence of SEQ ID NO: __, a vlCDR2 comprising the amino acid sequence of SEQ ID NO: __, and a vlCDR3 comprising the amino acid sequence of SEQ ID NO: __.
3. An antibody comprising a TL1A antigen-binding domain comprising a variable heavy domain and a variable light domain, wherein the variable heavy domain comprises the amino acid sequence of SEQ ID NO: __, and wherein the variable light domain comprises the amino acid sequence of SEQ ID NO: __.
4. The antibody of clause 1, wherein the antibody is a bispecific antibody.
5. The antibody of clause 4, wherein the bispecific antibody further comprises a means for binding IL23.

TABLE 55
below links the above listed Group 2 “means” clauses
to the relevant portions of the specification.

| Recited | Exemplified structures and/or materials and/or acts disclosed in the |
|---|---|
| function | Specification of the application filed herewith |
| Means | Any species of anti-IL23 antibody disclosed herein. |
| for | Species of anti-IL23 antibodies disclosed in WO2007005955, WO2007024846, |
| binding | WO2007027714, WO2007027761, WO2007076524, WO2007147019, WO2008103432, |
| IL23 | WO2008103473, WO2008134659, WO2008153610, WO2009082624, WO2010027766, |
| | WO2010142534, WO2011056600, WO2011159655, WO2012061448, WO2012093127, |
| | WO2013009535, WO2013165791, WO2013177101, WO2014004436, WO2014093203, |
| | WO2014093206, WO2014100312, WO2014137962, WO2014143540, WO2014149425, |
| | WO2015119841, WO2016014775, WO2016036918, WO2016073406, WO2016126638, |
| | WO2016168282, WO2017015433, WO2017048901, WO2017049035, WO2017074428, |
| | WO2017112536, WO2017172771, WO2017180821, WO2018064436, WO2018071504, |
| | WO2018093841, WO2018204156, WO2019027780, WO2019090329, WO2019171252, |
| | WO2019191464, WO2019215701, WO2020007368, WO2020012244, WO2020016838, |
| | WO2020055651, WO2020104943, WO2020108530, WO2020183418, WO2020198493, |
| | WO2020207724, WO2020212874, WO2020215019, WO2020219314, WO2020234834, |
| | WO2020245766, WO2020254826, WO2021035129, WO2021048743, WO2021126435, |
| | WO2021202731, WO2021224823, WO2021228113, WO2021234634, WO2021248718, |
| | WO2021260577, WO2022013745, WO2022056202, WO2022150472, WO2022187196, |
| | WO2022187657, WO2022190033, WO2022190034, WO2022243937, WO2022251623, |
| | WO2023036127, WO2023051798, WO2023051798, WO2023056417, WO2023064278, |
| | WO2023073615, WO2023084488, WO2023095000, WO2023111981, WO2023133538, |
| | WO2023135554, WO2023144699, WO2023187707, WO2023220663, WO2023223265, |
| | WO2023244746, WO2024026395, WO2024061288, WO2024077113, WO2024085697, |
| | WO2024110898, WO2024118630, WO2024178157, WO2024186635, WO2024191771, |
| | WO2024222891, WO2024228134, WO2024228135, WO2024233362, WO2024239006, |
| | WO2024241295, WO2024255867, WO2024263900, WO2025024723, WO2025032102, |
| | WO2025036280, WO2025051249, WO2025071362, WO2025071366, WO2025076414, |
| | WO2025083549, WO2025109545, WO2025111585, WO2025120601, WO2025137347, |
| | WO2025170982, and WO2025171006, each of which is incorporated by reference to the |
| | extent that the disclosure is of an anti-IL23 antibody. |

### Group 3 “Means” Clauses: Means for Binding TL1A, but IL23 Binding Domain Structurally Defined

### Exemplary Clauses:

1. An antibody comprising an IL23 antigen-binding domain comprising a variable heavy domain comprising a vhCDR1, a vhCDR2, and a vhCDR3 contained within a variable heavy domain comprising the amino acid sequence of SEQ ID NO: __, and a variable light domain comprising a vlCDR1, a vlCDR2, and a vlCDR3 contained within a variable light domain comprising the amino acid sequence of SEQ ID NO: __.
2. An antibody comprising an IL23 antigen-binding domain comprising a variable heavy domain comprising a vhCDR1 comprising the amino acid sequence of SEQ ID NO: __, a vhCDR2 comprising the amino acid sequence of SEQ ID NO: __, and a vhCDR3 comprising the amino acid sequence of SEQ ID NO: __, and a variable light domain comprising a vlCDR1 comprising the amino acid sequence of SEQ ID NO: __, a vlCDR2 comprising the amino acid sequence of SEQ ID NO: __, and a vlCDR3 comprising the amino acid sequence of SEQ ID NO: __.
3. An antibody comprising an IL23 antigen-binding domain comprising a variable heavy domain and a variable light domain, wherein the variable heavy domain comprises the amino acid sequence of SEQ ID NO: __, and wherein the variable light domain comprises the amino acid sequence of SEQ ID NO: __.
4. The antibody of clause 1, wherein the antibody is a bispecific antibody.
5. The antibody of clause 4, wherein the bispecific antibody further comprises a means for binding TL1A.

TABLE 56
below links the above listed Group 3 “means” clauses
to the relevant portions of the specification.

| Recited | Exemplified structures and/or materials and/or acts disclosed in the |
|---|---|
| function | Specification of the application filed herewith |
| Means | Any species of anti-TL1A antibody disclosed herein. |
| for | Species of anti-TLIA antibodies disclosed in WO2005018571, WO2006127900, |
| binding | WO2008106451, WO2009064854, WO2012064682, WO2013044298, WO2014106602, |
| TL1A | WO2014160883, WO2014186665, WO2014186750, WO2015010108, WO2015073580, |
| | WO2017049024, WO2017077715, WO2017106383, WO2017161342, WO2017196663, |
| | WO2017201461, WO2018022628, WO2018081074, WO2019209995, WO2020232125, |
| | WO2021081365, WO2021108694, WO2021202926, WO2021260577, WO2022103961, |
| | WO2022119842, WO2022140283, WO2022178158, WO2022178159, WO2022232253, |
| | WO2023009545, WO2023102051, WO2023133538, WO2023220663, WO2024026386, |
| | WO2024026395, WO2024112618, WO2024118630, WO2024137353, WO2024148218, |
| | WO2024148222, WO2024173838, WO2024173861, WO2024173865, WO2024173877, |
| | WO2024186859, WO2024233362, WO2024239006, WO2024249282, WO2024263868, |
| | WO2024263945, WO2025024723, WO2025038473, WO2025076081, WO2025111585, |
| | WO2025112237, WO2025140188, WO2025144089, WO2025160331, WO2025160334, and |
| | WO2025167705, each of which is incorporated by reference to the extent that the disclosure |
| | is of an anti-TL1A antibody. |

1. A bispecific antibody comprising:
(a) a TL1A-binding domain comprising a first variable heavy domain (VH1) and a first variable light domain (VL1); and
(b) an IL23-binding domain comprising a second variable heavy domain (VH2) and a second variable light domain (VL2).

2. The bispecific antibody of claim 1, wherein:
(a) the VH1 and VL1 are selected from:
(1) (i) a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in Table 44, optionally wherein the CDRs are as defined by the Kabat, Chothia, Kabat+Chothia, AbM, contact, or IMGT definition;
(ii) a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in Tables 7-10, 22, 23, 26, 29-34, and 48, optionally wherein the CDRs are as defined by the Kabat, Chothia, Kabat+Chothia, AbM, contact, or IMGT definition; and
(iii) a variable heavy domain comprising a vhCDR1, a vhCDR2, and a vhCDR3 contained within a variable heavy domain comprising the amino acid sequence of any one of SEQ ID NOS: 1427-1823 and 2316-2330, and a variable light domain comprising a vlCDR1, a vlCDR2, and a vlCDR3 contained within a variable light domain comprising the amino acid sequence of any one of 1824-1923, optionally wherein the CDRs are as defined by the Kabat, Chothia, Kabat+Chothia, AbM, contact, or IMGT definition;
(2) (i) any pair of anti-TL1A variable heavy domain and variable light domain in Table 44; and
(ii) any pair of anti-TL1A variable heavy domain and variable light domain in Tables 7-10, 22, 23, 26, 29-34, and 48; and
(iii) a variable heavy domain comprising the amino acid sequence of any one of SEQ ID NOS: 1427-1823 and 2316-2330, and a variable light domain comprising the amino acid sequence of any one of 1824-1923,
or variants thereof in which an N-terminal glutamine or glutamate is replaced with pyroglutamate; and/or
(b) the VH2 and VL2 are selected from:
(1) (i) a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in Table 44, optionally wherein the CDRs are as defined by the Kabat, Chothia, Kabat+Chothia, AbM, contact, or IMGT definition;
(ii) a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in Tables 11, 24-26, 29-34, and 49, optionally wherein the CDRs are as defined by the Kabat, Chothia, Kabat+Chothia, AbM, contact, or IMGT definition;
(iii) a variable heavy domain comprising a vhCDR1, a vhCDR2, and a vhCDR3 contained within a variable heavy domain comprising the amino acid sequence of any one of 1927-2132, and a variable light domain comprising a vlCDR1, a vlCDR2, and a vlCDR3 contained within a variable light domain comprising the amino acid sequence of any one of 2133-2232, optionally wherein the CDRs are as defined by the Kabat, Chothia, Kabat+Chothia, AbM, contact, or IMGT definition;
(2) (i) any pair of anti-IL23 variable heavy domain and variable light domain in Table 44; and
(ii) any pair of anti-IL23 variable heavy domain and variable light domain in Tables 11, 24-26, 29-34, and 49; and
(iii) a variable heavy domain comprising the amino acid sequence of any one of 1927-2132, and a variable light domain comprising the amino acid sequence of any one of 2133-2232,
or variants thereof in which an N-terminal glutamine or glutamate is replaced with pyroglutamate.

3. The bispecific antibody of claim 1, wherein the VH1 and the VL1 are selected from the following:
(a) a variable heavy domain comprising a vhCDR1, a vhCDR2, and a vhCDR3 contained within a variable heavy domain comprising the amino acid sequence of any one of SEQ ID NOS: 2241, 2253, 2259, 1674, and 1200, and a variable light domain comprising a vlCDR1, a vlCDR2, and a vlCDR3 contained within a variable light domain comprising the amino acid sequence of any one of SEQ ID NOS: 2243 and 1204;
(b) a variable heavy domain comprising a vhCDR1, a vhCDR2, and a vhCDR3 contained within a variable heavy domain comprising the amino acid sequence of SEQ ID NO: 2241, and a variable light domain comprising a vlCDR1, a vlCDR2, and a vlCDR3 contained within a variable light domain comprising the amino acid sequence of SEQ ID NO: 2243;
(c) a variable heavy domain comprising a vhCDR1, a vhCDR2, and a vhCDR3 contained within a variable heavy domain comprising the amino acid sequence of SEQ ID NO: 2253, and a variable light domain comprising a vlCDR1, a vlCDR2, and a vlCDR3 contained within a variable light domain comprising the amino acid sequence of SEQ ID NO: 2243;
(d) a variable heavy domain comprising a vhCDR1, a vhCDR2, and a vhCDR3 contained within a variable heavy domain comprising the amino acid sequence of SEQ ID NO: 2259, and a variable light domain comprising a vlCDR1, a vlCDR2, and a vlCDR3 contained within a variable light domain comprising the amino acid sequence of SEQ ID NO: 2243;
(e) a variable heavy domain comprising a vhCDR1, a vhCDR2, and a vhCDR3 contained within a variable heavy domain comprising the amino acid sequence of SEQ ID NO: 1674, and a variable light domain comprising a vlCDR1, a vlCDR2, and a vlCDR3 contained within a variable light domain comprising the amino acid sequence of SEQ ID NO: 1204;
(f) a variable heavy domain comprising a vhCDR1, a vhCDR2, and a vhCDR3 contained within a variable heavy domain comprising the amino acid sequence of SEQ ID NO: 1200, and a variable light domain comprising a vlCDR1, a vlCDR2, and a vlCDR3 contained within a variable light domain comprising the amino acid sequence of SEQ ID NO: 1204;
(g) a variable heavy domain comprising a vhCDR1, a vhCDR2, and a vhCDR3 contained within a variable heavy domain comprising the amino acid sequence of SEQ ID NO: 1661, and a variable light domain comprising a vlCDR1, a vlCDR2, and a vlCDR3 contained within a variable light domain comprising the amino acid sequence of SEQ ID NO: 1204;
(h) a variable heavy domain comprising a vhCDR1, a vhCDR2, and a vhCDR3 contained within a variable heavy domain comprising the amino acid sequence of SEQ ID NO: 2323, and a variable light domain comprising a vlCDR1, a vlCDR2, and a vlCDR3 contained within a variable light domain comprising the amino acid sequence of SEQ ID NO: 1204; and
(i) a variable heavy domain comprising a vhCDR1, a vhCDR2, and a vhCDR3 and a variable light domain comprising a vlCDR1, a vlCDR2, and a vlCDR3 contained within any pair of anti-TL1A variable heavy domain and variable light domain in XENP53412, XENP53413, XENP53369, XENP53375, XENP53387, XENP53415, XENP53370, XENP53371, XENP53376, XENP53377, XENP53378, XENP53379, XENP53380, or XENP53414,
optionally wherein the CDRs are as defined by the Kabat, Chothia, Kabat+Chothia, AbM, contact, or IMGT definition.

4.-69. (canceled)

70. A pharmaceutical composition comprising the bispecific antibody of claim 1 and a pharmaceutically acceptable carrier.

71. A kit, vessel, or delivery device comprising the bispecific antibody of claim 1.

72. (canceled)

73. A nucleic acid composition comprising a nucleic acid or a set of nucleic acids encoding the bispecific antibody of claim 1.

74. (canceled)

75. A host cell comprising the nucleic acid composition of claim 73.

76. A method of making a bispecific antibody, comprising culturing the host cell of claim 75 under conditions wherein the bispecific antibody is expressed, and recovering the bispecific antibody.

77. A method of inhibiting or reducing TL1A-mediated activity and/or IL23-mediated activity in a subject in need thereof, comprising administering to the subject the bispecific antibody of claim 1.

78.-80. (canceled)

81. A method of treating a TL1A-associated disease and/or IL23-associated disease in a subject in need thereof, comprising administering to the subject the bispecific antibody of claim 1.

82.-86. (canceled)

87. An antibody comprising a TNF-like ligand 1A (TL1A) antigen-binding domain comprising a variable heavy domain and a variable light domain selected from the following sets of variable heavy domains and variable light domains:
(a) a variable heavy domain comprising a vhCDR1, a vhCDR2, and a vhCDR3 contained within a variable heavy domain comprising the amino acid sequence of any one of SEQ ID NOS: 2241, 2253, 2259, 1674, and 1200, and a variable light domain comprising a vlCDR1, a vlCDR2, and a vlCDR3 contained within a variable light domain comprising the amino acid sequence of any one of SEQ ID NOS: 2243 and 1204;
(b) a variable heavy domain comprising a vhCDR1, a vhCDR2, and a vhCDR3 contained within a variable heavy domain comprising the amino acid sequence of SEQ ID NO: 2241, and a variable light domain comprising a vlCDR1, a vlCDR2, and a vlCDR3 contained within a variable light domain comprising the amino acid sequence of SEQ ID NO: 2243;
(c) a variable heavy domain comprising a vhCDR1, a vhCDR2, and a vhCDR3 contained within a variable heavy domain comprising the amino acid sequence of SEQ ID NO: 2253, and a variable light domain comprising a vlCDR1, a vlCDR2, and a vlCDR3 contained within a variable light domain comprising the amino acid sequence of SEQ ID NO: 2243;
(d) a variable heavy domain comprising a vhCDR1, a vhCDR2, and a vhCDR3 contained within a variable heavy domain comprising the amino acid sequence of SEQ ID NO: 2259, and a variable light domain comprising a vlCDR1, a vlCDR2, and a vlCDR3 contained within a variable light domain comprising the amino acid sequence of SEQ ID NO: 2243;
(e) a variable heavy domain comprising a vhCDR1, a vhCDR2, and a vhCDR3 contained within a variable heavy domain comprising the amino acid sequence of SEQ ID NO: 1674, and a variable light domain comprising a vlCDR1, a vlCDR2, and a vlCDR3 contained within a variable light domain comprising the amino acid sequence of SEQ ID NO: 1204;
(f) a variable heavy domain comprising a vhCDR1, a vhCDR2, and a vhCDR3 contained within a variable heavy domain comprising the amino acid sequence of SEQ ID NO: 1200, and a variable light domain comprising a vlCDR1, a vlCDR2, and a vlCDR3 contained within a variable light domain comprising the amino acid sequence of SEQ ID NO: 1204;
(g) a variable heavy domain comprising a vhCDR1, a vhCDR2, and a vhCDR3 contained within a variable heavy domain comprising the amino acid sequence of any one of SEQ ID NOS: 1674, 1200, 1661, and 2323, and a variable light domain comprising a vlCDR1, a vlCDR2, and a vlCDR3 contained within a variable light domain comprising the amino acid sequence of SEQ ID NO: 1204;
(h) a variable heavy domain comprising a vhCDR1, a vhCDR2, and a vhCDR3 contained within a variable heavy domain comprising the amino acid sequence of SEQ ID NO: 1661, and a variable light domain comprising a vlCDR1, a vlCDR2, and a vlCDR3 contained within a variable light domain comprising the amino acid sequence of SEQ ID NO: 1204;
(i) a variable heavy domain comprising a vhCDR1, a vhCDR2, and a vhCDR3 contained within a variable heavy domain comprising the amino acid sequence of SEQ ID NO: 2323, and a variable light domain comprising a vlCDR1, a vlCDR2, and a vlCDR3 contained within a variable light domain comprising the amino acid sequence of SEQ ID NO: 1204; and
(j) a variable heavy domain comprising a vhCDR1, a vhCDR2, and a vhCDR3 and a variable light domain comprising a vlCDR1, a vlCDR2, and a vlCDR3 contained within a variable heavy domain and variable light domain pair in XENP53412, XENP53413, XENP53369, XENP53375, XENP53387, XENP53415, XENP53370, XENP53371, XENP53376, XENP53377, XENP53378, XENP53379, XENP53380, or XENP53414,
optionally wherein the CDRs are as defined by the Kabat, Chothia, Kabat+Chothia, AbM, contact, or IMGT definition.

88.-102. (canceled)

103. A pharmaceutical composition comprising the antibody of claim 87 and a pharmaceutically acceptable carrier.

104. (canceled)

105. A kit, vessel, or delivery device comprising the antibody of claim 87.

106. (canceled)

107. A nucleic acid composition comprising a nucleic acid or a set of nucleic acids encoding the antibody of claim 87.

108. (canceled)

109. A host cell comprising the nucleic acid composition of claim 107.

110. A method of making an anti-TL1A antibody, comprising culturing the host cell of claim 109 under conditions wherein anti-TL1A antibody is expressed, and recovering the anti-TL1A antibody.

111. A method of inhibiting or reducing TL1A-mediated activity in a subject in need thereof, comprising administering to the subject the antibody of claim 87.

112.-114. (canceled)

115. A method of treating a TL1A-associated disease in a subject in need thereof, comprising administering to the subject the antibody of claim 87.

116.-124. (canceled)

125. An antibody comprising an interleukin-23 (IL23) antigen-binding domain comprising a variable heavy domain and a variable light domain selected from the following sets of variable heavy domains and variable light domains:
(a) a variable heavy domain comprising a vhCDR1, a vhCDR2, and a vhCDR3 contained within a variable heavy domain comprising the amino acid sequence of any one of SEQ ID NOS: 2233, 2235, and 179, and a variable light domain comprising a vlCDR1, a vlCDR2, and a vlCDR3 contained within a variable light domain comprising the amino acid sequence of any one of SEQ ID NOS: 2234, 2237, and 1248;
(b) a variable heavy domain comprising a vhCDR1, a vhCDR2, and a vhCDR3 contained within a variable heavy domain comprising the amino acid sequence of SEQ ID NO: 2233, and a variable light domain comprising a vlCDR1, a vlCDR2, and a vlCDR3 contained within a variable light domain comprising the amino acid sequence of SEQ ID NO: 2234;
(c) a variable heavy domain comprising a vhCDR1, a vhCDR2, and a vhCDR3 contained within a variable heavy domain comprising the amino acid sequence of SEQ ID NO: 2235, and a variable light domain comprising a vlCDR1, a vlCDR2, and a vlCDR3 contained within a variable light domain comprising the amino acid sequence of SEQ ID NO: 2237;
(d) a variable heavy domain comprising a vhCDR1, a vhCDR2, and a vhCDR3 contained within a variable heavy domain comprising the amino acid sequence of SEQ ID NO: 179, and a variable light domain comprising a vlCDR1, a vlCDR2, and a vlCDR3 contained within a variable light domain comprising the amino acid sequence of SEQ ID NO: 1248;
(e) a variable heavy domain comprising a vhCDR1, a vhCDR2, and a vhCDR3 contained within a variable heavy domain comprising the amino acid sequence of SEQ ID NO: 2113, and a variable light domain comprising a vlCDR1, a vlCDR2, and a vlCDR3 contained within a variable light domain comprising the amino acid sequence of SEQ ID NO: 2231;
(f) a variable heavy domain comprising a vhCDR1, a vhCDR2, and a vhCDR3 contained within a variable heavy domain comprising the amino acid sequence of SEQ ID NO: 1967, and a variable light domain comprising a vlCDR1, a vlCDR2, and a vlCDR3 contained within a variable light domain comprising the amino acid sequence of SEQ ID NO: 1248; and
(g) a variable heavy domain comprising a vhCDR1, a vhCDR2, and a vhCDR3 and a variable light domain comprising a vlCDR1, a vlCDR2, and a vlCDR3 contained within a variable heavy domain and variable light domain pair in XENP53412, XENP53413, XENP53369, XENP53375, XENP53387, XENP53415, XENP53370, XENP53371, XENP53376, XENP53377, XENP53378, XENP53379, XENP53380, or XENP53414, optionally wherein the CDRs are as defined by the Kabat, Chothia, Kabat+Chothia, AbM, contact, or IMGT definition.

126.-140. (canceled)

141. A pharmaceutical composition comprising the antibody of claim 125 and a pharmaceutically acceptable carrier.

142. (canceled)

143. A kit, vessel, or delivery device comprising the antibody of claim 125.

144. (canceled)

145. A nucleic acid composition comprising a nucleic acid or a set of nucleic acids encoding the antibody of claim 125.

146. (canceled)

147. A host cell comprising the nucleic acid composition of claim 145.

148. A method of making an anti-IL23 antibody, comprising culturing the host cell of claim 147 under conditions wherein anti-IL23 antibody is expressed, and recovering the anti-IL23 antibody.

149. A method of inhibiting or reducing IL23-mediated activity in a subject in need thereof, comprising administering to the subject the antibody of claim 125.

150.-152. (canceled)

153. A method of treating an IL23-associated disease in a subject in need thereof, comprising administering to the subject the antibody of claim 125.

154.-162. (canceled)

163. A combination comprising:
(a) a first antibody comprising a TNF-like ligand 1A (TL1A) antigen-binding domain comprising a variable heavy domain (VH1) and a variable light domain (VL1); and
(b) a second antibody comprising an interleukin-23 (IL23) antigen-binding domain comprising a variable heavy domain (VH2) and a variable light domain (VL2).

164.-197. (canceled)

198. A bispecific antibody comprising:
(a) a first monomer comprising a first heavy chain comprising a VH1-CH1-hinge-CH2-CH3, wherein VH1 is a first variable heavy domain, CH1-hinge-CH2-CH3 is a first heavy chain constant domain, and CH2-CH3 is a first Fc domain;
(b) a second monomer comprising a second heavy chain comprising a VH2-CH1-hinge-CH2-CH3, wherein VH2 is a second variable domain, CH1-hinge-CH2-CH3 is a second heavy chain constant domain, and CH2-CH3 is a second Fc domain;
(c) a first light chain comprising a VL1-CL, wherein VL1 is a first variable light domain, and CL is a first light chain constant domain; and
(d) a second light chain comprising a VL2-CL, wherein VL2 is a second variable light domain, and CL is a second light chain constant domain,
wherein the VH1 and VL1 together form a first antigen-binding domain (ABD), and the VH2 and the VL2 together form a second ABD,
wherein the first ABD is a TL1A-binding domain and the second ABD is an IL23-binding domain, or wherein the first ABD is an IL23-binding domain and the second ABD is a TL1A-binding domain, and wherein:
(i) the first heavy chain constant domain and the first light chain constant domain comprise a set of electrostatic steering variants at positions 213 and 218 of the first heavy chain constant domain according to EU numbering and positions 122 and 123 of the first light chain constant domain according to Kabat numbering, or
a set of steric skew variants at position 141 of the first heavy chain constant domain according to EU numbering and position 118 of the first light chain constant domain according to Kabat numbering, at position 141 of the first heavy chain constant domain according to EU numbering and position 116 of the first light chain constant domain according to Kabat numbering, or at position 147 of the first heavy chain constant domain according to EU numbering and position 131 of the first light chain constant domain according to Kabat numbering; and/or
(ii) the VH1 and the VL1 comprise a set of electrostatic steering variants at position 39 of VH1 and position 38 of VL1 according to Kabat numbering; and/or
(iii) the second heavy chain constant domain and the second light chain constant domain comprise a set of electrostatic steering variants at positions 213 and 218 of the second heavy chain constant domain according to EU numbering and positions 122 and 123 of the second light chain constant domain according to Kabat numbering, or
a set of steric skew variants at position 141 of the second heavy chain constant domain according to EU numbering and position 118 of the second light chain constant domain according to Kabat numbering, at position 141 of the second heavy chain constant domain according to EU numbering and position 116 of the second light chain constant domain according to Kabat numbering, or at position 147 of the second heavy chain constant domain according to EU numbering and position 131 of the second light chain constant domain according to Kabat numbering; and/or
(iv) the VH2 and the VL2 comprise a set of electrostatic steering variants at position 39 of VH2 and position 38 of VL2 according to Kabat numbering.

199. A pharmaceutical composition comprising the bispecific antibody of claim 198 and a pharmaceutically acceptable carrier.

200. A kit, vessel, or delivery device comprising the bispecific antibody of claim 198.

201. A nucleic acid composition comprising a nucleic acid or a set of nucleic acids encoding the bispecific antibody of claim 198.

202. A host cell comprising the nucleic acid composition of claim 201.

203. A method of making a bispecific antibody, comprising culturing the host cell of claim 202 under conditions wherein the bispecific antibody is expressed, and recovering the bispecific antibody.

204. A method of inhibiting or reducing TL1A-mediated activity and/or IL23-mediated activity in a subject in need thereof, comprising administering to the subject the bispecific antibody of claim 198.

205. A method of treating a TL1A-associated disease and/or IL23-associated disease in a subject in need thereof, comprising administering to the subject the bispecific antibody of claim 198.

206. A bispecific antibody comprising:
(a) a first monomer comprising a first heavy chain comprising a VL1-CH1-hinge-CH2-CH3, wherein VL1 is a first variable light domain, CH1-hinge-CH2-CH3 is a first heavy chain constant domain, and CH2-CH3 is a first Fc domain;
(b) a second monomer comprising a second heavy chain comprising a VH2-CH1-hinge-CH2-CH3, wherein VH2 is a second variable heavy domain, CH1-hinge-CH2-CH3 is a second heavy chain constant domain, and CH2-CH3 is a second Fc domain;
(c) a first light chain comprising a VH1-CL, wherein VH1 is a first variable heavy domain, and CL is a first light chain constant domain; and
(d) a second light chain comprising a VL2-CL, wherein VL2 is a second variable light domain, and CL is a second light chain constant domain,
wherein the VH1 and VL1 together form a first antigen-binding domain (ABD), and the VH2 and the VL2 together form a second ABD,
wherein the first ABD is a TL1A-binding domain and the second ABD is an IL23-binding domain, or wherein the first ABD is an IL23-binding domain and the second ABD is a TL1A-binding domain.

207. A bispecific antibody comprising:
(a) a means for binding TL1A; and
(b) a means for binding IL23.

208. A kit, vessel, or delivery device comprising the bispecific antibody of claim 207.

209. A nucleic acid composition comprising a nucleic acid or a set of nucleic acids encoding the bispecific antibody of claim 207.

210. A host cell comprising the nucleic acid composition of claim 209.

211. A method of making a bispecific antibody, comprising culturing the host cell of claim 210 under conditions wherein the bispecific antibody is expressed, and recovering the bispecific antibody.

212. A method of inhibiting or reducing TL1A-mediated activity and/or IL23-mediated activity in a subject in need thereof, comprising administering to the subject the bispecific antibody of claim 207.

213. A method of treating a TL1A-associated disease and/or IL23-associated disease in a subject in need thereof, comprising administering to the subject the bispecific antibody of claim 207.

214. A pharmaceutical composition comprising:
(a) a first composition comprising a means for binding TL1A; and
(b) a second composition comprising a means for binding IL23.

215. A pharmaceutical composition comprising:
(a) a means for binding TL1A;
(b) a means for binding IL23; and
(c) a pharmaceutically acceptable carrier.
